(19) United States
(12) Patent Application Publication
(10) Pub. No.: US 2024/0335414 A1
(43) Pub. Date: Oct. 10, 2024

(54) SPECIALIZED COMBINATIONS FOR MENTAL DISORDERS OR MENTAL ENHANCEMENT

(71) Applicant: TACTOGEN INC, Palo Alto, CA (US)

(72) Inventors: Matthew J. Baggott, Redwood City, CA (US); Sean Dalziel, Burlingame, CA (US)

(73) Assignee: TACTOGEN INC, Palo Alto, CA (US)

(21) Appl. No.: 18/737,589

(22) Filed: Jun. 7, 2024

Related U.S. Application Data
(63) Continuation of application No. PCT/US2022/052441, filed on Dec. 9, 2022.
(60) Provisional application No. 63/287,876, filed on Dec. 9, 2021.

(51) Int. Cl. A61K 31/343; A61K 9/20; A61K 31/137

(52) U.S. Cl. CPC A61K 31/343; A61K 9/2054; A61K 9/2086; A61K 9/2095; A61K 31/137

(57) ABSTRACT

The present invention discloses specialized combinations and methods of use thereof that may be used for beneficially modulating the central nervous system and for treating central nervous system, inflammatory, and metabolic disorders.

### CROSS-REFERENCE TO RELATED APPLICATIONS

This application is a continuation of International Patent Application No. PCT/US2022/052441, filed in the U.S. Receiving Office on Dec. 9, 2022, which claims the benefit of U.S. Provisional Application 63/287,876 filed on Dec. 9, 2021. The entirety of each of these applications is hereby incorporated by reference herein for all purposes.

### FIELD OF INVENTION

Specialized combinations and methods of use thereof that may be used for beneficially modulating the central nervous system and for treating central nervous system, inflammatory, and metabolic disorders are provided.

### BACKGROUND

Central nervous system (CNS) related health problems are a common challenge in society. An estimated 20.6% of U.S. adults (51.5 million people) experienced mental illness in 2019. This includes major depression (7.8% or 19.4 million people), anxiety disorders (19.1% or 48 million people), and posttraumatic stress disorder (PTSD) (3.6% or 9 million people). In addition to mental health challenges, there are other CNS disorders that cause substantial suffering and decreased quality of life. These include traumatic brain injury (TBI) (an estimated 12% of adults or 30 million people in the U.S.), dementias, and headache disorders (such as migraine, which affects about 15% of the general population or 47 million people in the U.S.). As the global population ages, many age-related CNS disorders are projected to become more common. For example, 6.2 million people aged 65 and older in the U.S. have Alzheimer's dementia and this population is expected to grow to 12.7 million by 2050.

There is a need for improved treatment of CNS disorders. Many patients fail to benefit adequately from available treatments. In addition, many available pharmacological treatments must be taken for weeks or months before the individual experiences therapeutic benefits. Because of these and other considerations, fewer than half of U.S. adults with mental illness (44.8%) received treatment in 2019.

Entactogens (sometimes called empathogens) have become the focus of attention to as a tool to help solve some of these serious health problems. They increase feelings of authenticity and emotional openness while decreasing social anxiety (Baggott et al., Journal of Psychopharmacology 2016, 30.4: 378-87). Entactogens have potential for cultivating intimacy, open communication, and interpersonal healing in human relationships. This is in part because they seem to allow individuals to engage in emotionally meaningful activity with lessened influence from trauma history, attachment patterns, and self-criticism.

Entactogens are typically monoamine releasers that appear to produce their effects in part by increasing extracellular serotonin in the brain, which both stimulates hypothalamic serotonergic receptors, thus triggering release of the hormone oxytocin, and also stimulates serotonergic 5-HT_{1B} receptors on cells in the nucleus accumbens area of the brain. However, these drugs do have varying and complex effects that result from binding to a range of 5-HT and other receptors. Entactogens can be distinguished from drugs that are primarily hallucinogenic or psychedelic, and amphetamines, which are primarily stimulants.

The most well-known entactogen, MDMA (3,4-methylenedioxymethamphetamine), is currently in human clinical trials in the United States and Europe. It is being tested as an aid to psychotherapy sessions for PTSD and has been more broadly suggested as useful for aiding social cognition (Preller & Vollenweider, Frontiers in Psychiatry, 2019, 10; Hysek et al., Social cognitive and affective neuroscience, 2015, 9.11, 1645-52). The FDA granted breakthrough therapy designation for the PTSD program and has also agreed to an expanded access program, both indicative of promising results. (Feduccia et al., Frontiers in Psychiatry, 2019, 10: 650; Sessa et al., Frontiers in Psychiatry, 2019, 10: 138; see also the MDMA Investigator's Brochure, 14th Edition: Mar. 18, 2022, and references therein, available from the sponsor of MDMA clinical trials at MAPS.org). Indeed, in the first of two Phase 3 trials using MDMA-assisted therapy to treat PTSD, two-thirds of participants in the MDMA arm had sufficient improvement that they were no longer diagnosable with PTSD (Mitchell et al. 2021. Nature Medicine. 27(6):1025-33).

Although MDMA appears to have significant therapeutic value, it has some features that reduce its acceptability to patients and may limit its clinical uses. For example, many patients find that the initial effects are moderately unpleasant. These initial effects can include nausea (sometimes including vomiting) and anxiety. In a pooled analysis of Phase 2 clinical trials, nausea occurred in 40.3% (29 of 72) of participants, while anxiety occurred in 72.2% (52 of 72) of participants (eTable 6 in Mithoefer et al. 2019. Psychopharmacology. 236(9):2735-45). Additionally, MDMA can produce feelings of dizziness, sedation, drunkenness, difficulty concentrating, and mild confusion during the first five hours after drug administration. These and other undesired effects can distract the patient from both the therapeutic effects of the drug and psychotherapy or other activities taking place during the hours after administering MDMA.

There is also considerable variability in the effects of MDMA. The acute psychological and emotional effects of MDMA have a duration reported as averaging 4.2 hours with a standard deviation of 1.3 hours after 75 or 125 mg MDMA by Vizeli & Liechti (2017. Journal of Psychopharmacology, 31(5), 576-588). Part of the variability in duration is likely due to relatively high inter-person variation in the pharmacokinetics of MDMA and metabolites such as HMMA (4-hydroxy-3-methoxymethamphetamine) (Kolbrich et al. 2008. Therapeutic drug monitoring. 30(3):320). Some of this variability in kinetics may be because MDMA is both a substrate and inhibitor of cytochrome p450 isozyme 2D6 (CYP2D6).

Patent applications describing entactogenic compounds include WO 2021/252538, WO 2022/010937, WO 2022/032147, and WO 2022/061242 which are assigned to Tactogen Inc. Additional patent applications describing entactogenic compounds and methods of using entactogenic compounds include but are not limited to U.S. Pat. No. 7,045,545, WO 2005/058865, WO 2020/169850, WO 2020/169851, WO 2021/257169, WO 2021/225796, WO 2022/214889, WO 2022/120181, WO 2022/072808, and WO 2022/038171.

Clinical studies in healthy volunteers have explored the interactions of MDMA and different drugs. In one study, the dopamine uptake inhibitor methylphenidate and MDMA were studied alone and in combination (Hysek et al. 2014. International journal of neuropsychopharmacology, 17(3), pp. 371-381). Co-administering methylphenidate and MDMA did not produce more psychoactive effects compared with either drug alone, but potentially enhanced cardiovascular and adverse effects. Given these results with a dopamine reuptake inhibitor, the current invention is unexpected.

It is an object of the present invention to provide advantageous compositions and their use and manufacture for improving the therapeutic effects of entactogens, including increasing desired effects and decreasing undesired effects.

### SUMMARY OF THE INVENTION

The present invention includes specialized combinations and methods for administering entactogens that can increase the proportion of desired effects compared to undesired effects. These methods and preparations are based on the finding that the pharmacological and therapeutic profiles of entactogens and related drugs can be improved by changing the timing by which increases of different brain monoamines occur. To accomplish the desired timing, in certain aspects, a pharmaceutical composition is provided comprising granules formulated for immediate release with the first therapeutic agent and granules formulated for delayed release with the second therapeutic agent. For example, in certain embodiments a bilayer tablet is provided comprising an inner and outer layer wherein the inner layer comprises (1) immediate release granules comprising a dopamine releasing agent and one or more pharmaceutically acceptable excipients; (2) delayed release granules comprising an entactogen and one or more pharmaceutically acceptable excipients; and (3) one or more additional pharmaceutically acceptable excipients; wherein the outer layer is a film coating.

In certain aspects of the invention a combination of an entactogenic agent and a dopamine releasing agent is provided wherein the plasma. In certain embodiments the kinetic lag is a later Tmax of one agent than the other. For example, the Tmax of one of the agents may be greater than at least about 5, 10, 15, 20, 25, 30, 35, 40, 45, 50, 55, 60, 90, or 120 minutes later than the other agent. In certain embodiments the kinetic lag is a later Cmax or 50% Cmax than the other agent for example one of the agents may have a Cmax or 50% Cmax that is at least about 5, 10, 15, 20, 25, 30, 35, 40, 45, 50, 55, 60, 90, or 120 minutes later than the other agent.

In certain embodiments a combination of an entactogenic agent and a dopamine releasing agent is administered so that one agent has a later Tpeak than the other, wherein Tpeak is defined as the time wherein the plasma concentration reaches at least 50%, 60%, 70%, 80%, 90%, or 100% of Cmax, preferably from about 80% to about 100%, and most preferably about 80% of Cmax. For example, the Tpeak of any of the active agent may be about 15, 20, 25, 30, 45, 60, 90, 120, 150, 180, or about 240 minutes or less after administration.

In certain aspects of the present invention the effects of the dopamine releasing agent are felt by the patient first and then the effects of the entactogenic compound are felt. In other aspects of the present invention the effects of the entactogenic compound are felt by the patient first and then the effects of the dopamine releasing agent are felt. In certain embodiments two entactogenic compounds are given to the patient where the release of one is delayed providing a more favorable therapeutic experience. For example, in certain embodiments one of the two entactogenic compounds has a higher dopamine release than the other and by releasing this entactogen first a better patient response can be achieved.

Entactogens are compounds that can increase extracellular concentrations of serotonin in the brain with high potency (i.e., less than 10 μM EC₅₀, preferably less than 1 μM EC₅₀, most preferably less than 250 nM EC₅₀), and that, when taken in effective doses via effective routes of administration, typically produces an altered state of consciousness including generally positive mood, decreased neuroticism, increased authenticity, and increased emotional and social openness in a person. Entactogens usually have some ability to increase extracellular dopamine, with their dopamine to serotonin EC₅₀ ratio being usually less than five. Ability to increase extracellular serotonin and dopamine can be measured by in vitro or ex vivo assays, such as those described later, that use radiolabeled neurotransmitter (or an appropriate substitute, such as 1-methyl-4-phenylpyridinium, MPP+).

Non-limiting examples of entactogens include MDMA, MDA (3,4-methylenedioxyamphetamine), MDAI (2H,5H,6H,7H-indeno[5,6-d][1,3]dioxol-6-amine), BK-MDMA (1-(2H-1,3-benzodioxol-5-yl)-2-(methylamino)propan-1-one), BK-MDEA (1-(2H-1,3-benzodioxol-5-yl)-2-(ethylamino)propan-1-one), MBDB ([1-(2H-1,3-benzodioxol-5-yl)butan-2-yl](methyl)amine), butylone (1-(2H-1,3-benzodioxol-5-yl)-2-(methylamino)butan-1-one), eutylone (1-(2H-1,3-benzodioxol-5-yl)-2-(ethylamino)butan-1-one), MDEA ([1-(2H-1,3-benzodioxol-5-yl)propan-2-yl](ethyl)amine), αMT (1-(1H-indol-3-yl)propan-2-amine), α,N-DMT ([1-(1H-indol-3-yl)propan-2-yl](methyl)amine), BK-NM-AMT (1-(1H-indol-3-yl)-2-(methylamino)propan-1-one), 5-F-NM-AMT ([1-(5-fluoro-1H-indol-3-yl)propan-2-yl](methyl)amine), BK-5-F-NM-AMT (1-(5-fluoro-1H-indol-3-yl)-2-(methylamino)propan-1-one), 2-APB (1-(1-benzofuran-2-yl)propan-2-amine), 5-APB (1-(1-benzofuran-5-yl)propan-2-amine), 6-APB ((1-(1-benzofuran-6-yl)propan-2-amine), APBI (5H,6H,7H-indeno[5,6-b]furan-6-amine), 2-MAPB ([1-(1-benzofuran-2-yl)propan-2-yl](methyl)amine), 5-MAPB ([1-(1-benzofuran-5-yl)propan-2-yl](methyl)amine), 6-MAPB ([1-(1-benzofuran-6-yl)propan-2-yl](methyl)amine), 2-EAPB ([1-(1-benzofuran-2-yl)propan-2-yl](ethyl)amine), 5-EAPB ([1-(1-benzofuran-2-yl)propan-5-yl](ethyl)amine), 6-EAPB ([1-(1-benzofuran-6-yl)propan-2-yl](ethyl)amine), 2-MBPB ([1-(1-benzofuran-2-yl)butan-2-yl](methyl)amine), 5-MBPB ([1-(1-benzofuran-5-yl)butan-2-yl](methyl)amine), 6-MBPB ([1-(1-benzofuran-6-yl)butan-2-yl](methyl)amine), BK-5-MAPB (1-(1-benzofuran-5-yl)-2-(methylamino)propan-1-one), BK-2-MAPB (1-(1-benzofuran-2-yl)-2-(methylamino)propan-1-one), FLEA (N-[1-(2H-1,3-benzodioxol-5-yl)propan-2-yl]-N-methylhydroxylamine), and MDOH (N-[1-(2H-1,3-benzodioxol-5-yl)propan-2-yl]hydroxylamine). Additional agents can be identified using the assays described herein or their equivalent. When agents are referred to herein as freebase a pharmacologically acceptable salt of the agent is also contemplated except when excluded by context.

In certain aspects the entactogen is:

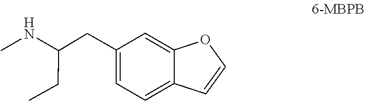

or a salt or salt mixture thereof, optionally as an enantiomerically pure or enantiomerically enriched mixture.

In certain aspects the entactogen is selected from:

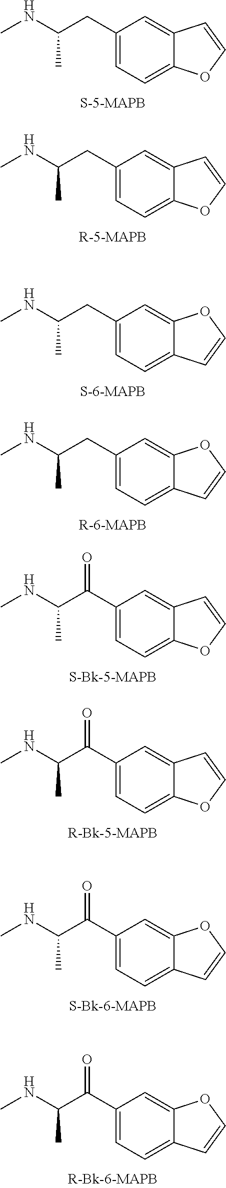

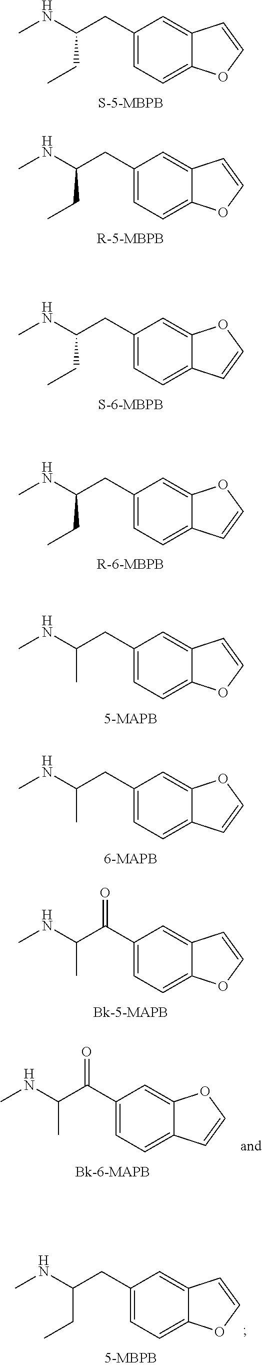

or a salt or salt mixture thereof, optionally as an enantiomerically pure or enantiomerically enriched mixture.

In certain aspects the entactogen is selected from:

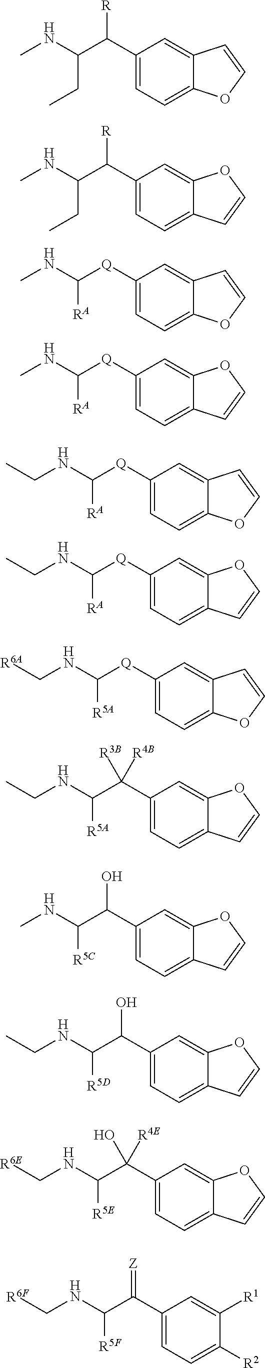

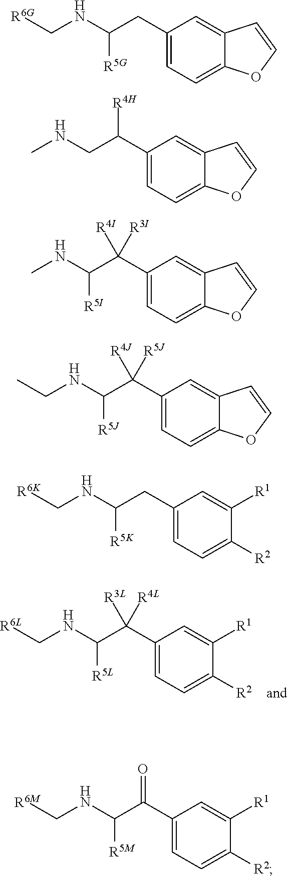

or a salt or salt mixture thereof, optionally as an enantiomerically pure or enantiomerically enriched mixture;
wherein:
R is hydrogen or hydroxyl;
R^{A} is —CH₃, —CH₂Y, —CHY₂, —CY₃, —CH₂CH₃, —CH₂CH₂Y, —CH₂CHY₂, —CH₂CY₃, —CH₂OH, or —CH₂CH₂OH;
Q is selected from:

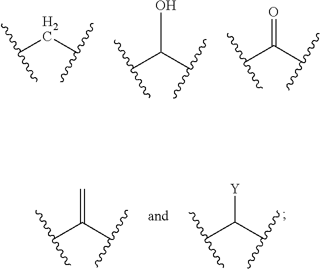

Y is halogen;
R¹ and R² are taken together as —OCH═CH— or —CH═CHO—;
R^{3B} and R^{4B} are independently selected from —H, —X, C₁-C₄ alkyl, —CH₂OH, —CH₂X, —CHX₂, and —CX₃, wherein at least one of R^{3B} and R^{4B} is not —H;
R^{3L} and R^{4L} are independently selected from —H, —X, —OH, C₁-C₄ alkyl, —CH₂OH, —CH₂X, —CHX₂, and —CX₃, wherein at least one of R^{3I} and R^{4I} is not —H;
R^{3I} and R^{4I} are independently selected from —H, —X, —OH, —CH₂OH, —CH₂X, —CHX₂, —CX₃, and C₁-C₄ alkyl; wherein at least one of R^{3I} and R^{4I} is not —H;
R^{3J} and R^{4J} are independently selected from —H, —X, —OH, C₁-C₄ alkyl, —CH₂OH, —CH₂X, —CHX₂, and —CX₃;
R^{4E} is selected from C₁-C₄ alkyl, —CH₂OH, —CH₂X, —CHX₂, and —CX₃;
R^{4H} is selected from —X, —CH₂CH₂CH₃, —CH₂OH, —CH₂X, and —CHX₂;
R^{5A} and R^{5G} are independently selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₂-C₄ alkyl, when R^{5A} is C₂ alkyl or H, R^{6A} is not —H, and when R^{5G} is —H or C₂ alkyl, R^{6G} is not —H;
R^{5B} is selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₁-C₄ alkyl;
R^{5C} is selected from —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₂-C₄ alkyl;
R^{5D}, R^{5E}, R^{5F}, and R^{5J} are independently selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₁-C₄ alkyl, when R^{5F} is —H or C₁ alkyl, R^{6F} cannot be —H, and when R^{5J} is C₁ alkyl, at least one of R^{3J} and R^{4J} is not H;
R^{5K} is selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₂-C₄ alkyl;
R^{5L} and R^{5M} are independently selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₁-C₄ alkyl; and
R^{5I} is selected from —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₁-C₄ alkyl; wherein at least one of R^{3I}, R^{4I}, and R^{5I} is not C₁ alkyl;
R^{6A}, R^{6B}, R^{6E}, R^{6F}, and R^{6G} are independently selected from —H and —CH₃;
R^{6K}, R^{6L}, and R^{6M} are independently selected from —H and —CH₃;
X is independently selected from —F, —Cl, and —Br; and
Z is selected from O and CH₂.

In certain aspects the entactogen is selected from:

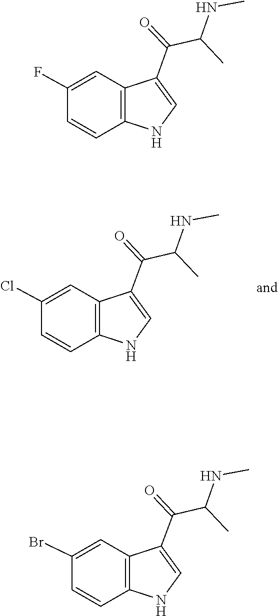 [SMILES: CNC(C)C(=O)c1c[nH]c2ccc(F)cc12 ; CNC(C)C(=O)c1c[nH]c2ccc(Cl)cc12 ; CNC(C)C(=O)c1c[nH]c2ccc(Br)cc12]

or a prodrug, salt, or salt mixture thereof, optionally as an enantiomerically pure or enantiomerically enriched mixture.

In certain aspects the entactogen is selected from:

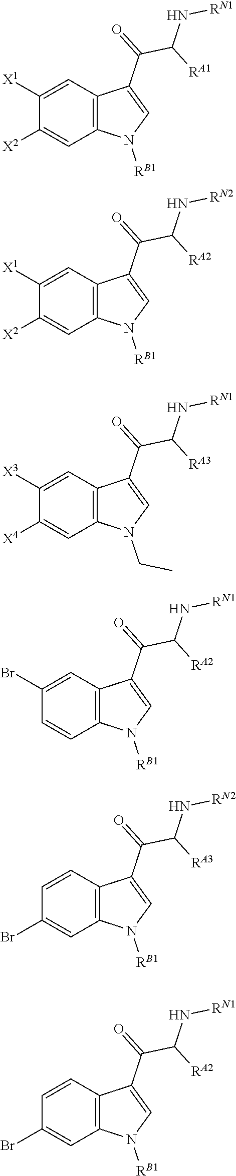

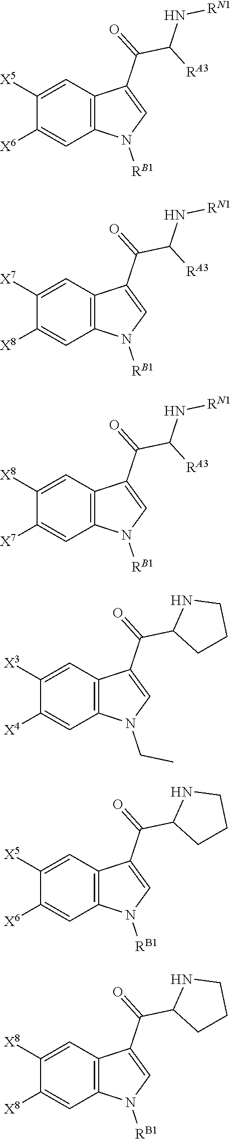

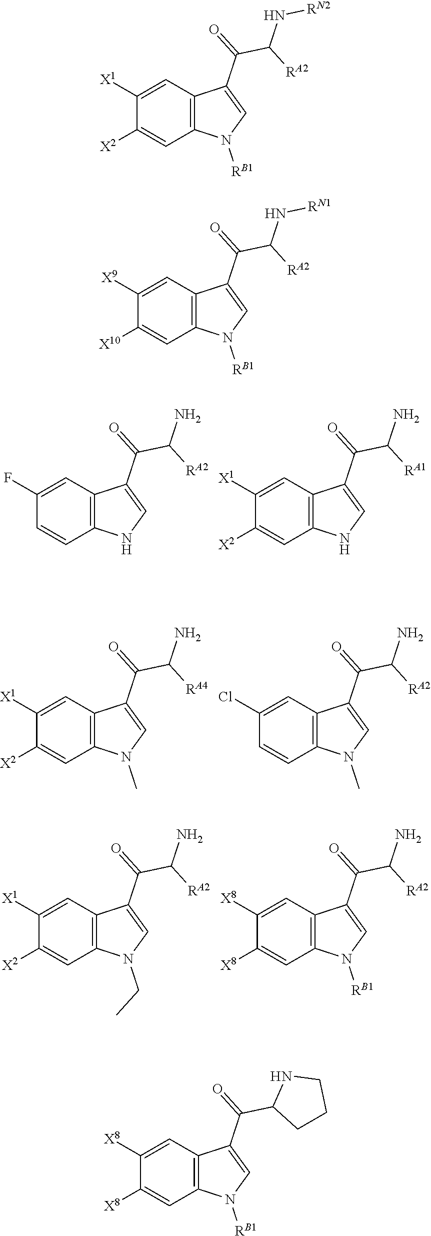

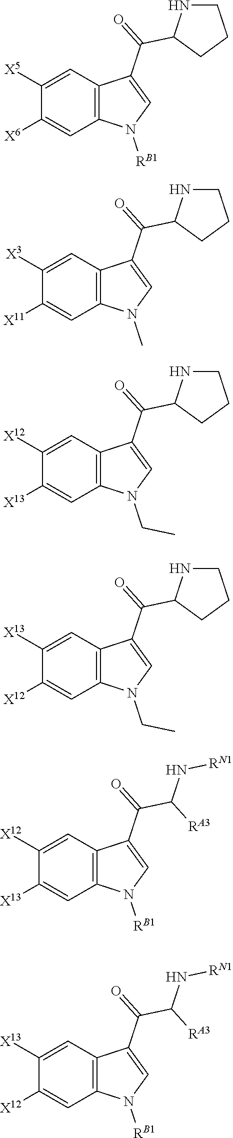

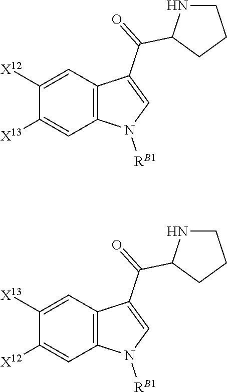

or a prodrug, salt, or salt mixture thereof, optionally as an enantiomerically pure or enantiomerically enriched mixture;
wherein:
R^{N1} is selected from —H, —CH₃, and —CH₂CH₃;
R^{N2} is selected from —CH₃, and —CH₂CH₃;
R^{A1} is selected from —CH₂CH₂X, —CH₂CHX₂, —CH₂CX₃, —CH₂OH and —CH₂CH₂OH;
R^{A2} is selected from —CH₃, —CH₂CH₃, —CH₂CH₂X, —CH₂CHX₂, —CH₂CX₃, —CH₂OH and —CH₂CH₂OH;
R^{A3} is selected from —H, —CH₃, —CH₂CH₃, —CH₂CH₂X, —CH₂CHX₂, —CH₂CX₃, —CH₂OH and —CH₂CH₂OH;
R^{B1} is selected from —H, —CH₃, and —CH₂CH₃;
X¹ is independently selected in each instance from —H, —F and —Cl;
X² is selected from —H, —F and —Cl, wherein X¹ and X² must be different;
X³ is selected from —H, —F, —Cl, and —Br;
X⁴ is selected from —H, —F, —Cl, and —Br, wherein X³ and X⁴ must be different;
X⁵ is selected from —H and —I;
X⁶ is selected from —H and —I, wherein X⁵ and X⁶ must be different;
X⁷ is selected from —F, —Br, and —I;
X⁸ is independently selected in each instance from —F, —Cl, —Br, and —I; and
X is independently selected in each instance from —F, —Cl, —Br, and —I.

Additional examples of entactogens include

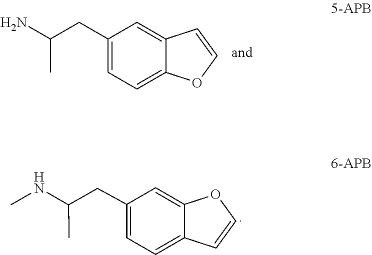

Dopamine-releasing agents are those that can increase extracellular concentrations of dopamine with high potency (i.e., less than 10 μM EC₅₀, preferably less than 1 μM EC₅₀, most preferably less than 250 nM EC₅₀) in appropriate assays, as described herein. Examples of dopamine-releasing agents include amphetamine, fencamfamine, phenmetrazine, 2-fluorophenmetrazine, 3-fluorophenmetrazine, methamphetamine (methyl[1-(5,6,7,8-tetrahydronaphthalen-2-yl)propan-2-yl]amine), naphthylaminopropane, 5-(2-Aminopropyl)indole, methcathinone, 2-methyl-methcathinone, 3-methyl-methcathinone, 4-methyl-methcathinone (4-MMC), 3-fluoroamphetamine, 3-fluoromethcathinone, 4-fluoroamphetamine, 4-fluoromethcathinone, 3-bromoamphetamine, 3-bromomethcathinone, 4-bromoamphetamine, 4-bromomethcathinone, N-methylamphetamine, N-benzyl-methamphetamine, 3-methylamphetamine, 4-methylamphetamine, N,4-dimethylamphetamine, 2-(Methylamino)-1-naphthalen-1-ylpropan-1-one, methylthioamphetamine, and N,N-dimethyl-thioamphetamine. Additional agents can be identified using the assays described herein or their equivalent.

In certain embodiments, two pharmacologically active agents are coadministered so that one has an earlier Tmax than the other. In other embodiments, two pharmacologically active agents are coadministered so that one reaches 50% of its plasma Cmax before the other. Both this difference in Tmax and difference in 50% Cmax will be hereafter referred to as a kinetic lag. Illustrative kinetic lags that are contemplated include less than 5, 5, 10, 15, 20, 25, 30, 35, 40, 45, 50, 55, 60, 90, and 120 minutes or more, as well as values in between these numbers.

In certain embodiments, two pharmacologically active agents are coadministered so that one has an earlier Tpeak than the other. In other embodiments, two pharmacologically active agents are coadministered so that one reaches at least 50%, 60%, 70%, 80%, 90%, or at least 100% of its plasma Cmax before the other.

In certain embodiments, this kinetic lag is achieved by using oral dosage and controlled release. Methods for achieving controlled release with orally administered drugs are detailed below.

In other embodiments, this kinetic lag is achieved by oral dosage combined with parenteral administration (such as buccal, sublingual, inhaled, intramuscular, intravenous, and other parenteral routes of administration) to provide a rapid increase in plasma and brain concentrations of one or more agent.

In some embodiments, the two pharmacologically active agents consist of an entactogen and an agent that can increase extracellular dopamine in the brain. For example, RS-MDMA may be administered with amphetamine, methamphetamine, methylphenidate, phenmetrazine, another dopamine releasing agent, or a derivative or prodrug.

In certain embodiments, dopamine-releasing agents are preferred if they have a higher dopamine to serotonin EC₅₀ ratio than the entactogenic agent with which they are co-administered.

In other embodiments, dopamine-releasing agents are preferred if they have a lower dopamine EC₅₀ than the entactogenic agent with which they are co-administered.

In certain embodiments, a chiral entactogenic compound is administered so that one enantiomer has an earlier Tmax than the other enantiomer. For example, an oral preparation may use controlled release so that there is an earlier Tmax for the enantiomer that has a higher dopamine to serotonin EC₅₀ ratio compared to the other enantiomer. Thus, R-4-MMC and S-4-MMC may be administered so that R-4-MMC has an earlier Tmax.

In certain embodiments, a chiral entactogenic compound is administered so that one enantiomer has an earlier Tpeak than the other enantiomer. For example, an oral preparation may use controlled release so that there is an earlier Tpeak for the enantiomer that has a higher dopamine to serotonin EC₅₀ ratio compared to the other enantiomer. Thus, R-4-MMC and S-4-MMC may be administered so that R-4-MMC has an earlier Tpeak.

Altering the time-varying balance of dopaminergic versus serotonergic effects produces changes in the therapeutic profile of entactogens. In some embodiments, fewer early undesirable effects occur. In some embodiments, patients experience greater acute therapeutic effects and can make greater therapeutic progress. In some embodiments, lower doses can be used to achieve therapeutic effects that are normally produced by higher doses of the entactogen.

In certain aspects a third active agent is released after the immediate and delayed release agents. For example, in certain embodiments a pharmaceutical composition is provided that immediately releases a dopamine release and then after a kinetic lag releases an entactogen followed by release of a third active agent. To accomplish the desired timing, in certain aspects, a pharmaceutical composition is provided comprising granules formulated for immediate release with the first therapeutic agent; granules formulated for delayed release with the second therapeutic agent; and granules formulated for a second delayed release which are coated with an enteric coating. For example, in certain embodiments a bilayer tablet is provided comprising an inner and outer layer wherein the inner layer comprises (1) immediate release granules comprising a dopamine releasing agent and one or more pharmaceutically acceptable excipients; (2) delayed release granules comprising an entactogen and one or more pharmaceutically acceptable excipients; (3) enterically coated granules comprising a dual serotonin-norepinephrine reuptake inhibitor and one or more pharmaceutically acceptable excipients; and (4) one or more additional pharmaceutically acceptable excipients; wherein the outer layer is a film coating.

Non-limiting examples of specialized combinations of the present invention include:

1.

| Agent | Amount | PK/PD |
|---|---|---|
| S-MDMA | Between about 10 mg | Immediate release (for example |
| | and about 180 mg | an oral Tmax of less than about |
| | | 120, 90, or 60 minutes). |
| R-MDMA | Between about 10 mg | Delayed release. Oral Tmax |
| | and about 180 mg | at about 15 minutes after |
| | | Tmax of Agent 1 |

2.

| Agent | Amount | PK/PD |
|---|---|---|
| Amphetamine | Between about 2 mg | Immediate release (for example |
| | and about 40 mg | an oral Tmax of less than about |
| | | 120, 90, or 60 minutes). |
| S-MDMA | Between about 10 mg | Delayed release. Oral Tmax |
| | and about 200 mg | at about 15 minutes after |
| | | Tmax of Agent 1 |
| Milnacipran | Between about 10 mg | Delayed release. Starting |
| | and about 100 mg | at about 120 minutes to |
| | | about 360 minutes |

3.

| Agent | Amount | PK/PD |
|---|---|---|
| Amphetamine | Between about 2 mg and about | Immediate release (for example an oral |
| | 40 mg | Tmax of less than about 120, 90, or 60 |
| | | minutes). |
| S-MDMA | Between about 10 mg and | Delayed release. Oral Tmax at about 15 |
| | about 200 mg | minutes after Tmax of Agent 1 |
| Citalopram | Between about 20 mg and | Delayed release. Starting at about 120 |
| | about 40 mg | minutes to about 360 minutes |

4.

| Agent | Amount | PK/PD |
|---|---|---|
| Amphetamine | Between about 2 mg and about | Immediate release (for example an oral |
| | 40 mg | Tmax of less than about 120, 90, or 60 |
| | | minutes). |
| S-MDMA | Between about 10 mg and | Delayed release. Oral Tmax at about 15 |
| | about 200 mg | minutes after Tmax of Agent 1 |
| Duloxetine | Between about 20 mg and | Delayed release. Starting at about 120 |
| | about 60 mg | minutes to about 360 minutes |

5.

| Agent | Amount | PK/PD |
|---|---|---|
| Amphetamine | Between about 2 mg and about | Immediate release (for example an oral |
| | 40 mg | Tmax of less than about 120, 90, or 60 |
| | | minutes). |
| S-5-MAPB | Between about 5 mg and about | Delayed release. Oral Tmax at about 15 |
| | 120 mg | minutes after Tmax of Agent 1 |

6.

| Agent | Amount | PK/PD |
|---|---|---|
| Amphetamine | Between about 2 mg and about | Immediate release (for example an oral |
| | 40 mg | Tmax of less than about 120, 90, or 60 |
| | | minutes). |
| S-5-MAPB | Between about 5 mg and about | Delayed release. Oral Tmax at about 15 |
| | 120 mg | minutes after Tmax of Agent 1 |
| Milnacipran | Between about 10 mg and | Delayed release. Starting at about 120 |
| | about 100 mg | minutes to about 360 minutes |

7.

| Agent | Amount | PK/PD |
|---|---|---|
| Amphetamine | Between about 2 mg and about | Immediate release (for example an oral |
| | 40 mg | Tmax of less than about 120, 90, or 60 |
| | | minutes). |
| S-5-MAPB | Between about 5 mg and about | Delayed release. Oral Tmax at about 15 |
| | 120 mg | minutes after Tmax of Agent 1 |
| Citalopram | Between about 20 mg and | Delayed release. Starting at about 120 |
| | about 40 mg | minutes to about 360 minutes |

8.

| Agent | Amount | PK/PD |
|---|---|---|
| Amphetamine | Between about 2 mg and about | Immediate release (for example an oral |
| | 40 mg | Tmax of less than about 120, 90, or 60 |
| | | minutes). |
| S-5-MAPB | Between about 5 mg and about | Delayed release. Oral Tmax at about 15 |
| | 120 mg | minutes after Tmax of Agent 1 |
| Duloxetine | Between about 20 mg and | Delayed release. Starting at about 120 |
| | about 60 mg | minutes to about 360 minutes |

9.

| Agent | Amount | PK/PD |
|---|---|---|
| R-BK-MDMA | Between about 5 mg and about | Immediate release (for example an oral |
| | 300 mg | Tmax of less than about 120, 90, or 60 |
| | | minutes). |
| S- BK-MDMA | Between about 5 mg and about | Delayed release. Oral Tmax at about 15 |
| | 300 mg | minutes after Tmax of Agent 1 |

10.

| Agent | Amount | PK/PD |
|---|---|---|
| S-5-APB | Between about 5 mg and about | Immediate release (for example an oral |
| | 100 mg | Tmax of less than about 120, 90, or 60 |
| | | minutes). |
| R-5-APB | Between about 5 mg and about | Delayed release. Oral Tmax at about 15 |
| | 100 mg | minutes after Tmax of Agent 1 |

11.

| Agent | Amount | PK/PD |
|---|---|---|
| S-6-APB | Between about 5 mg and about | Immediate release (for example an oral |
| | 100 mg | Tmax of less than about 120, 90, or 60 |
| | | minutes). |
| R-6-APB | Between about 5 mg and about | Delayed release. Oral Tmax at about 15 |
| | 100 mg | minutes after Tmax of Agent 1 |

12.

| Agent | Amount | PK/PD |
|---|---|---|
| R-BK-5-MAPB | Between about 5 mg and about | Immediate release (for example an oral |
| | 150 mg | Tmax of less than about 120, 90, or 60 |
| | | minutes). |
| S-BK-5-MAPB | Between about 5 mg and about | Delayed release. Oral Tmax at about 15 |
| | 150 mg | minutes after Tmax of Agent 1 |

13.

| Agent | Amount | PK/PD |
|---|---|---|
| S-6-MBPB | Between about 5 mg and about | Immediate release (for example an oral |
| | 120 mg | Tmax of less than about 120, 90, or 60 |
| | | minutes). |
| R-6-MBPB | Between about 5 mg and about | Delayed release. Oral Tmax at about 15 |
| | 120 mg | minutes after Tmax of Agent 1 |

14.

| Agent | Amount | PK/PD |
|---|---|---|
| S-5-MBPB | Between about 5 mg and about | Immediate release (for example an oral |
| | 120 mg | Tmax of less than about 120, 90, or 60 |
| | | minutes). |
| R-5-MBPB | Between about 5 mg and about | Delayed release. Oral Tmax at about 15 |
| | 120 mg | minutes after Tmax of Agent 1 |

### DETAILED DESCRIPTION OF TABLE INVENTION

The present invention includes specialized combinations and methods for administering entactogens that can increase the proportion of desired effects compared to undesired effects. These methods and preparations are based on the finding that the pharmacological and therapeutic profiles of entactogens can be improved in unexpected ways by changing the timing by which increases of different brain monoamines occur.

The embodiments of the invention are presented to meet the goal of assisting persons with mental disorders, who desire mental enhancement, or who suffer from other CNS disorders by providing mild therapeutics that are fast acting and that reduce the properties that decrease the patient experience, are counterproductive to the therapy, or are undesirably toxic. One goal of the invention is to provide therapeutic compositions that increase empathy, sympathy, openness and acceptance of oneself and others, which can be taken, if necessary, as part of therapeutic counseling sessions, when necessary, episodically or even consistently, as prescribed by a healthcare provider.

### Definitions

When introducing elements of the present invention or the preferred embodiments thereof, the articles “a,” “an,” “the,” and “said” are intended to mean that there are one or more of the elements. The terms “comprising,” “including,” and “having” are intended to be inclusive and not exclusive (i.e., there may be other elements in addition to the recited elements). Thus, the terms “including,” “may include,” and “include,” as used herein mean, and are used interchangeably with, the phrase “including but not limited to.”

Where a range of values is provided, it is understood that the upper and lower limit, and each intervening value between the upper and lower limit of the range is encompassed within the embodiments.

Unless defined otherwise, all technical and scientific terms herein have the meaning as commonly understood by one of ordinary skill in the art to which this invention belongs. In the event there is a plurality of definitions for a term herein, those in this section prevail unless stated otherwise. Further definitions that may assist the reader to understand the disclosed embodiments are as follows, and such definitions may be used to interpret the defined terms, when those terms are used herein. However, the examples given in the definitions are generally non-exhaustive and must not be construed as limiting the invention. It also will be understood that a substituent should comply with chemical bonding rules and steric compatibility constraints in relation to the particular molecule to which it is attached.

“Entactogen” and “entactogenic compound” is defined herein as a drug that can increase extracellular concentrations of serotonin in the brain with high potency (i.e., less than 10 μM EC₅₀, preferably less than 1 μM EC₅₀, most preferably less than 250 nM EC₅₀), and that, when taken in effective doses via effective routes of administration, typically produces an altered state of consciousness including generally positive mood, decreased neuroticism, increased authenticity, and increased emotional and social openness in a person. Entactogens generally have some ability to increase extracellular dopamine, with their dopamine to serotonin EC₅₀ ratio being usually less than 5.

Dopamine to serotonin EC₅₀ ratio is defined herein as 1/EC₅₀ for releasing dopamine via DAT divided by 1/EC₅₀ for releasing serotonin via SERT. Ability to increase extracellular serotonin and dopamine can be measured by in vitro or ex vivo assays, such as those described later, that use radiolabeled neurotransmitter (or an appropriate substitute, such as 1-methyl-4-phenylpyridinium, MPP+). Increases of serotonin, dopamine, and other neurotransmitters are often expressed in comparison to the effects of another drug such as norfenfluramine (for serotonin), dextroamphetamine (for dopamine), or tyramine (for serotonin, dopamine, and norepinephrine) and are given as a percent of the maximum increase produced by the comparator drug. This convention is used herein as well.

In certain embodiments the entactogen is MDMA. In addition to MDMA, non-exhaustive examples of entactogens include MDA (3,4-methylenedioxyamphetamine), MDAI (2H,5H,6H,7H-indeno[5,6-d][1,3]dioxol-6-amine), BK-MDMA (1-(2H-1,3-benzodioxol-5-yl)-2-(methylamino)propan-1-one), BK-MDEA (1-(2H-1,3-benzodioxol-5-yl)-2-(ethylamino)propan-1-one), MBDB ([1-(2H-1,3-benzodioxol-5-yl)butan-2-yl](methyl)amine), butylone (1-(2H-1,3-benzodioxol-5-yl)-2-(methylamino)butan-1-one), eutylone (1-(2H-1,3-benzodioxol-5-yl)-2-(ethylamino)butan-1-one), MDEA ([1-(2H-1,3-benzodioxol-5-yl)propan-2-yl](ethyl)amine), aMT (1-(1H-indol-3-yl)propan-2-amine), α,N-DMT ([1-(1H-indol-3-yl)propan-2-yl](methyl)amine), BK-NM-AMT (1-(1H-indol-3-yl)-2-(methylamino)propan-1-one), 5-F-NM-AMT ([1-(5-fluoro-1H-indol-3-yl)propan-2-yl](methyl)amine), BK-5-F-NM-AMT (1-(5-fluoro-1H-indol-3-yl)-2-(methylamino)propan-1-one), 2-APB (1-(1-benzofuran-2-yl)propan-2-amine), 5-APB (1-(1-benzofuran-5-yl)propan-2-amine), 6-APB ((1-(1-benzofuran-6-yl)propan-2-amine), APBI (5H,6H,7H-indeno[5,6-b]furan-6-amine), 2-MAPB ([1-(1-benzofuran-2-yl)propan-2-yl](methyl)amine), 5-MAPB ([1-(1-benzofuran-5-yl)propan-2-yl](methyl)amine), 6-MAPB ([1-(1-benzofuran-6-yl)propan-2-yl](methyl)amine), 2-EAPB ([1-(1-benzofuran-2-yl)propan-2-yl](ethyl)amine), 5-EAPB ([1-(1-benzofuran-2-yl)propan-5-yl](ethyl)amine), 6-EAPB ([1-(1-benzofuran-6-yl)propan-2-yl](ethyl)amine), 2-MBPB ([1-(1-benzofuran-2-yl)butan-2-yl](methyl)amine), 5-MBPB ([1-(1-benzofuran-5-yl)butan-2-yl](methyl)amine), 6-MBPB ([1-(1-benzofuran-6-yl)butan-2-yl](methyl)amine), BK-5-MAPB (1-(1-benzofuran-5-yl)-2-(methylamino)propan-1-one), BK-2-MAPB (1-(1-benzofuran-2-yl)-2-(methylamino)propan-1-one), BK-6-EAPB (1-(1-benzofuran-6-yl)-2-(ethylamino)propan-1-one), BK-5-EAPB (1-(1-benzofuran-5-yl)-2-(ethylamino)propan-1-one), BK-2-EAPB (1-(1-benzofuran-2-yl)-2-(ethylamino)propan-1-one), FLEA (N-[1-(2H-1,3-benzodioxol-5-yl)propan-2-yl]-N-methylhydroxylamine), aF-5-MAPB ([1-(1-benzofuran-5-yl)-3-fluoropropan-2-yl](methyl)amine), aF-6-MAPB ([1-(1-benzofuran-6-yl)-3-fluoropropan-2-yl](methyl)amine), and MDOH (N-[1-(2H-1,3-benzodioxol-5-yl)propan-2-yl]hydroxylamine). Additional agents can be identified using the assays described herein or their equivalent. (When agents are referred to herein either a freebase or a pharmacologically acceptable salt is intended.)

Dopamine-releasing agents are those that can increase extracellular concentrations of dopamine with high potency (i.e., less than 10 μM EC₅₀, preferably less than 1 μM EC₅₀, most preferably less than 250 nM EC₅₀) in appropriate assays, as described herein. Examples of dopamine-releasing agents include amphetamine, fencamfamine, phenmetrazine, 2-fluorophenmetrazine, 3-fluorophenmetrazine, metamnetamine (methyl[1-(5,6,7,8-tetrahydronaphthalen-2-yl)propan-2-yl]amine), naphthylaminopropane, 5-(2-Aminopropyl)indole, methcathinone, 2-methyl-methcathinone, 3-methyl-methcathinone, 4-methyl-methcathinone (4-MMC), 3-fluoroamphetamine, 3-fluoromethcathinone, 4-fluoroamphetamine, 4-fluoromethcathinone, 3-bromoamphetamine, 3-bromomethcathinone, 4-bromoamphetamine, 4-bromomethcathinone, N-methylamphetamine, N-benzyl-methamphetamine, 3-methylamphetamine, 4-methylamphetamine, N,4-dimethylamphetamine, 2-(Methylamino)-1-naphthalen-1-ylpropan-1-one, methylthioamphetamine, and N,N-dimethyl-thioamphetamine. Additional agents can be identified using the assays described herein or their equivalent.

The current invention finds unexpected benefits from specific methods of coadministration of an entactogen and a dopamine-releasing agent. Coadministration herein refers to administering one or more agents such that they have overlapping plasma-concentration-versus-time curves and where the agents (or active metabolite(s) in the case of prodrugs) are first detectable in plasma within 3 hours of each other. In other words, the ascending limbs of the plasma-concentration-versus-time curves are separated by 3 hours or less. Coadministration is intended to exclude cases where one agent is being used chronically, such as daily. Thus, we exclude administration of an entactogen to someone who is regularly taking a dopamine-releasing agent, such as to treat ADHD.

“Alkyl” in certain specific embodiments refers to a saturated or unsaturated, branched, straight-chain, or cyclic monovalent hydrocarbon radical derived by the removal of one hydrogen atom from a single carbon atom of a parent alkane, alkene or alkyne. Typical alkyl groups include methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. Alkyl will be understood to include cyclic alkyl radicals such as cyclopropyl, cyclobutyl, and cyclopentyl.

“Alkyl” in certain specific embodiments includes radicals having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. Preferably, an alkyl group comprises from 1 to 26 carbon atoms, more preferably, from 1 to 10 carbon atoms.

“Halogen” or “halo” means fluoro (F), chloro (Cl), bromo (Br), or iodo (I). For groups containing two or more halogens, such as CHX₂ or CX₃, and for example “where X is halogen,” it will be understood that each Y independently will be selected from the group of halogens.

“Hydroxy” means the radical —OH.

In certain embodiments tableting excipients include common pharmaceutical excipients selected from diluents, binders, compression aids, granulating agents, disintegrants, glidants, tablet coatings and films, coloring agents, non-brittle filler, super-disintegrant, enteric polymer coating agents and carrier particles. Non-limiting examples of tableting excipients include sugar compounds such as lactose, dextrin, glucose, sucrose, sorbitol; inorganic compounds such as silicates, calcium and magnesium salts, sodium or potassium chloride; synthetic polymers such as starches, sugars, sugar alcohols, and cellulose derivatives; hydrophilic compounds which swell or dissolve in water such as alginates, crospovidone, croscarmellose sodium; colloidal anhydrous silicon and silica compounds; stearic acid and its salts such as magnesium stearate; sugar based coating agents, natural or synthetic polymers such as cellulose acetate phthalate, Eudragit® FL 30-D and sugar spheres, microcrystalline cellulose; synthetic dyes, and natural food pigments.

### Embodiments of “Alkyl”

Unless otherwise specifically referenced “alkyl” is a branched, straight chain, or cyclic saturated aliphatic hydrocarbon group. In certain embodiments, the alkyl from 1 to about 6 carbon atoms, from 1 to about 4 carbon atoms, or from 1 to 3 carbon atoms. In certain embodiments, the alkyl contains from 1 to about 8 carbon atoms. In certain embodiments, the alkyl is C₁-C₂, C₁-C₃, C₁-C₄, C₁-C₅ or C₁-C₆. The specified ranges as used herein indicate an alkyl group which is considered to explicitly disclose as individual species each member of the range described as a unique species. For example, the term C₁-C₆ alkyl as used herein indicates a straight or branched alkyl group having 1, 2, 3, 4, 5, or 6 carbon atoms and also a carbocyclic alkyl group of 3, 4, 5, or 6 carbon atoms and is intended to mean that each of these is described as an independent species. For example, the term C₁-C₄alkyl as used herein indicates a straight or branched alkyl group having 1, 2, 3, or 4 carbon atoms and is intended to mean that each of these is described as an independent species. Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, 2,3-dimethylbutane, and hexyl.

In certain embodiments “alkyl” is a C₁-C₆alkyl, C₁-C₃alkyl, C₁-C₄alkyl, C₁-C₃alkyl, or C₁-C₂alkyl.

In certain embodiments “alkyl” has one carbon.

In certain embodiments “alkyl” has two carbons.

In certain embodiments “alkyl” has three carbons.

In certain embodiments “alkyl” has four carbons.

In certain embodiments “alkyl” has five carbons.

In certain embodiments “alkyl” has six carbons.

Non-limiting examples of “alkyl” include: methyl, ethyl, propyl, butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

In certain embodiments when a term is used that includes “alk” it should be understood that “cycloalkyl” or “carbocyclic” can be considered part of the definition, unless unambiguously excluded by the context.

### METHODS OF THE PRESENT INVENTION

The current invention discloses unexpected benefits from specific methods of coadministration of an entactogen and a dopamine-releasing agent. In certain embodiments, two pharmacologically active agents are coadministered so that one has an earlier Tmax than the other. In other embodiments, two pharmacologically active agents are coadministered so that one reaches 50% of its plasma Cmax before the other. Both this difference in Tmax and difference in 50% Cmax will be hereafter referred to as a kinetic lag.

In certain embodiments kinetic lags that are contemplated include less than 5, 5, 10, 15, 20, 25, 30, 35, 40, 45, 50, 55, 60, 90, and 120 minutes or more, as well as values in between these numbers.

In certain embodiments, this kinetic lag is achieved by using oral dosage and controlled release. Methods for achieving controlled release with orally administered drugs are detailed below.

In other embodiments, this kinetic lag is achieved by oral dosage combined with parenteral administration (such as buccal, sublingual, inhaled, intramuscular, intravenous, and other parenteral routes of administration) to provide a rapid increase in plasma and brain concentrations of one or more agent.

In certain embodiments Tmax of at least one of the active agents is measured by in vivo methods, wherein Tmax generally refers to the time at which the maximum concentration of the drug is detected in plasma.

In certain other embodiments, wherein the active agent exhibits slower absorption (Tmax>3 hours) from the digestive tract to general circulation, Tmax of at least one of the active agents is preferably measured with USP apparatus.

In certain embodiments, two pharmacologically active agents are coadministered so that one has an earlier Tpeak than the other. In other embodiments, two pharmacologically active agents are coadministered so that one reaches at least 50%, 60%, 70%, 80%, 90%, or at least 100% of its plasma Cmax before the other.

In some embodiments, the two pharmacologically active agents consist of an entactogen and an agent that can increase extracellular dopamine in the brain. For example, RS-MDMA may be administered with amphetamine, methamphetamine, methylphenidate, phenmetrazine, another dopamine releasing agent, or a derivative or prodrug, so that RS-MDMA has a later Tmax.

In certain embodiments, dopamine-releasing agents are preferred if they have a higher dopamine to serotonin EC₅₀ ratio than the entactogenic agent with which they are co-administered.

In other embodiments, dopamine-releasing agents are preferred if they have a lower dopamine EC₅₀ than the entactogenic agent with which they are co-administered.

In certain embodiments a chiral entactogenic compound is administered so that one enantiomer has kinetic lag compared to the other enantiomer. For example, an oral preparation may use controlled release so that there is an earlier Tmax (or Time of 50% Cmax) for the enantiomer that has a higher dopamine to serotonin EC₅₀ ratio compared to the other enantiomer. Thus, R-4-MMC and S-4-MMC may be administered so that R-4-MMC is earlier with S-4-MMC lagged in comparison (dopamine and serotonin release profiles for 4-MMC are in Gregg et al. 2015. British journal of pharmacology, 172(3), pp. 883-894).

Altering the time-varying balance of dopaminergic versus serotonergic effects produces changes in the therapeutic profile of entactogens. In some embodiments, fewer early undesirable effects occur. Undesirable symptoms of an entactogen include nausea, vomiting, headache, sedation, difficulty concentrating, lack of appetite, lack of energy, and decreased mood.

In some embodiments, patients experience greater acute therapeutic effects and can make greater therapeutic progress. In some embodiments, lower doses can be used to achieve therapeutic effects that are normally produced by higher doses of the entactogen. Exemplary methods of assessing these effects are provided herein.

In certain aspects the invention provides a method described below:
1. A method of enhancing the therapeutic profile of an entactogenic agent, involving co-administration of a dopamine releasing agent, consisting of producing a combination of decreases in undesired effects and increases in desired effects by creating a kinetic lag between the agents.
2. A method of 1 where the dopamine releasing agent has a DAT to SERT EC₅₀ ratio lower than the entactogen.
3. A method of 1 where the dopamine releasing agent has a lower DAT EC₅₀ than the entactogen.
4. A method of 1-3 where the kinetic lag is a difference in Tmax of the agents is any of less than 5, 5, 10, 15, 20, 25, 30, 35, 40, 45, 50, 55, 60, 90, and 120 minutes or more.
5. A method of 1-3 where the kinetic lag is a difference in timing of the 50% Cmax of the agents is any of less than 5, 5, 10, 15, 20, 25, 30, 35, 40, 45, 50, 55, 60, 90, and 120 minutes or more.
6. A method of 1-5 where the entactogen and dopamine releasing agents are in the same dosage form and have different release profiles.
7. A method of 1-5 where the entactogen and dopamine releasing agents are in the different dosage forms and one dosage form uses parenteral administration.
8. A method of 7 where the dosage forms are oral and inhaled.
9. A method of 7 where the dosage forms are oral and injected (such as intravenous and intramuscular).
10. A method of 7 where the dosage forms are oral and buccal or sublingual.
11. A method of 1-10 where the entactogen is MDMA and the dopamine releasing agents are one or more of amphetamine, and methamphetamine, or prodrugs of the same.
12. A method of 1-10 where the entactogen is 5-MAPB and the dopamine releasing agents are one or more of amphetamine, and methamphetamine, or prodrugs of the same.
13. A method of 1-10 where the entactogen is MDMA and the dopamine releasing agent is BK-MDMA.
14. A method of 1-10 where the entactogen is enantiomerically enriched 6-MBPB (with more S than R-enantiomer) and the dopamine releasing agent is enantiomerically enriched 6-MBPB (with more R- than S-enantiomer).
15. A method of 1-10 where the entactogen is enantiomerically enriched 5-MBPB (with more S than R-enantiomer) and the dopamine releasing agent is enantiomerically enriched 5-MBPB (with more R- than S-enantiomer).
16. A method of 1-10 where the entactogen is R-5-MAPB and the dopamine releasing agent is S-5-MAPB.
17. A method of 1-10 where the entactogen is 5-MAPB and the dopamine releasing agent is amphetamine, and methamphetamine, or prodrugs of the same.
18. A method of 1-10 where the entactogen is MDMA and the dopamine releasing agent is 4-MMC.
19. A method of 1-10 where the entactogen is S-4-MMC and the dopamine releasing agent is R-4-MMC.
20. A method of 1-10 where the entactogen is R-MDA and the dopamine releasing agent is S-MDA.
21. A method of 1-20 where the decreased undesired effects are any of nausea, vomiting, anxiety, dizziness, sedation, drunkenness, difficulty concentrating, and mild confusion.
22. A method of 1-20 where the desired effects are decreased neuroticism, increased authenticity, and positive mood.
23. A method of 1-22 where the kinetic lag is a difference in Tpeak of the agents is any of less than 15, 20, 25, 30, 35, 40, 45, 50, 55, 60, 90, and 120 minutes or more.
24. In certain embodiments Tpeak is the time wherein the plasma concentration reaches at least 50% Cmax.
25. In certain embodiments Tpeak is the time wherein the plasma concentration reaches at least 60% Cmax.
26. In certain embodiments Tpeak is the time wherein the plasma concentration reaches at least 70% Cmax.
27. In certain embodiments Tpeak is the time wherein the plasma concentration reaches at least 80% Cmax.
28. In certain embodiments Tpeak is the time wherein the plasma concentration reaches at least 90% Cmax.
29. In certain embodiments Tpeak is the time wherein the plasma concentration reaches at least 100% Cmax.
30. In certain embodiments Tpeak is the time wherein the plasma concentration reaches at least 50% Cmax.
31. In certain embodiments Tpeak is the time wherein the plasma concentration reaches at least 60% Cmax.
32. In certain embodiments Tpeak is the time wherein the plasma concentration reaches at least 70% Cmax.
33. In certain embodiments Tpeak is the time wherein the plasma concentration reaches at least 80% Cmax.
34. In certain embodiments Tpeak is the time wherein the plasma concentration reaches at least 90% Cmax.
35. In certain embodiments Tpeak is the time wherein the plasma concentration reaches at least 100% Cmax.
36. Any one of embodiments 24-35, wherein the Tpeak for the immediate release agent is about 15 minutes.
37. Any one of embodiments 24-35, wherein the Tpeak for the immediate release agent is about 20 minutes.
38. Any one of embodiments 24-35, wherein the Tpeak for the immediate release agent is about 25 minutes.
39. Any one of embodiments 24-35, wherein the Tpeak for the immediate release agent is about 30 minutes.
40. Any one of embodiments 24-35, wherein the Tpeak for the immediate release agent is about 45 minutes.
41. Any one of embodiments 24-35, wherein the Tpeak for the immediate release agent is about 60 minutes.
42. Any one of embodiments 24-35, wherein the Tpeak for the immediate release agent is about 90 minutes.
43. Any one of embodiments 24-35, wherein the Tpeak for the immediate release agent is about 120 minutes.
44. Any one of embodiments 24-35, wherein the Tpeak for the immediate release agent is about 150 minutes.
45. Any one of embodiments 24-35, wherein the Tpeak for the immediate release agent is about 180 minutes.
46. Any one of embodiments 24-35, wherein the Tpeak for the immediate release agent is about 240 minutes or less after administration.
47. Any one of embodiments 24-46, wherein the Tpeak for the delayed release agent is about 15 minutes after the Tpeak of the immediate release agent.
48. Any one of embodiments 24-46, wherein the Tpeak for the delayed release agent is about 20 minutes after the Tpeak of the immediate release agent.
49. Any one of embodiments 24-46, wherein the Tpeak for the delayed release agent is about 25 minutes after the Tpeak of the immediate release agent.
50. Any one of embodiments 24-46, wherein the Tpeak for the delayed release agent is about 30 minutes after the Tpeak of the immediate release agent.
51. Any one of embodiments 24-46, wherein the Tpeak for the delayed release agent is about 45 minutes after the Tpeak of the immediate release agent.
52. Any one of embodiments 24-46, wherein the Tpeak for the delayed release agent is about 60 minutes after the Tpeak of the immediate release agent.
53. Any one of embodiments 47-52, wherein the immediate release agent is a dopamine releasing agent.
54. Any one of embodiments 47-53, wherein the delayed release agent is an entactogen.
55. Any one of embodiments 47-54, wherein the delayed release agent is a dopamine releasing agent.
56. Any one of embodiments 47-55, wherein the immediate release agent is an entactogen.

The present invention provides methods and uses for the treatment of CNS disorders, including, but not limited to, mental disorders as described herein, including post-traumatic stress and adjustment disorders, comprising administering a combination of an entactogenic compound or a pharmaceutically acceptable salt or mixture of salts thereof and a dopamine releasing agent, or a pharmaceutically acceptable salt or mixture of salts thereof as described herein. These combinations display many pharmacological properties that are beneficial to their use as therapeutics and represent an improvement over existing therapeutics.

In certain embodiments a combination is provided wherein both agents are entactogenic and they are administered with a kinetic lag.

The present invention also provides, for example, methods for the treatment of disorders, including, but not limited to depression, dysthymia, anxiety and phobia disorders (including generalized anxiety, social anxiety, panic, post-traumatic stress and adjustment disorders), feeding and eating disorders (including binge eating, bulimia, and anorexia nervosa), other binge behaviors, body dysmorphic syndromes, alcoholism, tobacco abuse, drug abuse or dependence disorders, disruptive behavior disorders, impulse control disorders, gaming disorders, gambling disorders, memory loss, dementia of aging, attention deficit hyperactivity disorder, personality disorders (including antisocial, avoidant, borderline, histrionic, narcissistic, obsessive compulsive, paranoid, schizoid and schizotypal personality disorders), attachment disorders, autism, and dissociative disorders.

In addition to treating various diseases and disorders, the employed methods of modulating activity of the serotonergic system in particular can be used to improve CNS functioning in non-disease states, such as reducing neuroticism and psychological defensiveness, increasing openness to experience, increasing creativity, and aiding decision-making.

In other embodiments, an entactogen and a dopamine release agent combination of the present invention is provided in an effective amount to treat a host, typically a human, with a CNS disorder that can be either a neurological condition (one that is typically treated by a neurologist) or a psychiatric condition (one that is typically treated by a psychiatrist). Neurological disorders are typically those affecting the structure, biochemistry or cause electrical abnormalities of the brain, spinal cord or other nerves. Psychiatric conditions are more typically thought of as mental disorders, which are primarily abnormalities of thought, feeling or behavior that cause significant distress or impairment of personal functioning.

Thus, the disclosed compounds can be used in an effective amount to improve neurological or psychiatric functioning in a patient in need thereof. Neurological indications include, but are not limited to improved neuroplasticity, including treatment of stroke, brain trauma, dementia, and neurodegenerative diseases. MDMA has been reported to have an EC₅₀ of 7.41 nM for promoting neuritogenesis and an Emax approximately twice that of ketamine, which has fast acting psychiatric benefits that are thought to be mediated by its ability to promote neuroplasticity, including the growth of dendritic spines, increased synthesis of synaptic proteins, and strengthening synaptic responses. Figure S3. in Ly et al. (Cell reports 23, no. 11 (2018): 3170-3182, https://doi.org/10.1016/j.celrep2018.05.022). In certain embodiments the compounds used in the current invention can similarly be considered psychoplastogens, that is, small molecules that are able to induce rapid neuroplasticity (Olson, 2018, Journal of experimental neuroscience, 12, 1179069518800508. https://doi.org/10.1177%2F1179069518800508). For example, in certain embodiments, the disclosed compounds and compositions can be used to improve stuttering and other dyspraxias or to treat Parkinson's disease or schizophrenia.

The term “improving psychiatric function” is intended to include mental health and life conditions that are not traditionally treated by neurologists but sometimes treated by psychiatrists and can also be treated by psychotherapists, life coaches, personal fitness trainers, meditation teachers, counselors, and the like. For example, it is contemplated that the disclosed compounds will allow individuals to effectively contemplate actual or possible experiences that would normally be upsetting or even overwhelming. This includes individuals with fatal illness planning their last days and the disposition of their estate. This also includes couples discussing difficulties in their relationship and how to address them. This also includes individuals who wish to more effectively plan their careers.

In other embodiments, an entactogen and a dopamine release agent combination of the present invention may be used in an effective amount to treat a host, typically a human, to modulate an immune or inflammatory response. The compounds disclosed herein alter extracellular serotonin, which is known to alter immune functioning. MDMA produces acute time-dependent increases and decreases in immune response.

The following nonlimiting examples are relevant to any of the disorders, indications, methods of use or dosing regimes described herein. When a host is treated with an effective amount of an enantiomerically enriched mixture of enantiomers of a compound described herein that treatment can either be in the form of one enantiomer being the immediate release agent and the other being the delayed release agent or both enantiomers can be dosed together as an immediate release agent and followed by another agent described herein after a kinetic lag. In certain embodiments the enantiomerically enriched mixture of enantiomers is the immediate release agent. In certain embodiments the enantiomerically enriched mixture of enantiomers is the second agent which follows the first after a kinetic lag.

In certain embodiments, a host, for example a human, is treated with an effective amount of an enantiomerically enriched mixture of enantiomers of a compound described herein, or a pharmaceutically acceptable salt or mixed salt, isotopic derivative, or prodrug thereof, wherein the percent of S enantiomer is greater than about 99 percent.

In certain embodiments, a host, for example a human, is treated with an effective amount of an enantiomerically enriched mixture of enantiomers of a compound described herein, or a pharmaceutically acceptable salt or mixed salt, isotopic derivative, or prodrug thereof, wherein the percent of S enantiomer is greater than about 95 percent.

In certain embodiments, a host, for example a human, is treated with an effective amount of an enantiomerically enriched mixture of enantiomers of a compound described herein, or a pharmaceutically acceptable salt or mixed salt, isotopic derivative, or prodrug thereof, wherein the percent of S enantiomer is greater than about 90 percent.

In certain embodiments, a host, for example a human, is treated with an effective amount of an enantiomerically enriched mixture of enantiomers of a compound described herein, or a pharmaceutically acceptable salt or mixed salt, isotopic derivative, or prodrug thereof, wherein the percent of S enantiomer is greater than about 85 percent.

In certain embodiments, a host, for example a human, is treated with an effective amount of an enantiomerically enriched mixture of enantiomers of a compound described herein, or a pharmaceutically acceptable salt or mixed salt, isotopic derivative, or prodrug thereof, wherein the percent of S enantiomer is greater than about 80 percent.

In certain embodiments, a host, for example a human, is treated with an effective amount of an enantiomerically enriched mixture of enantiomers of a compound described herein, or a pharmaceutically acceptable salt, mixed salt, isotopic derivative, or prodrug thereof, wherein the percent of S enantiomer is greater than about 75 percent.

In certain embodiments, a host, for example a human, is treated with an effective amount of an enantiomerically enriched mixture of enantiomers of a compound described herein, or a pharmaceutically acceptable salt, mixed salt, isotopic derivative, or prodrug thereof, wherein the percent of S enantiomer is greater than about 70 percent.

In certain embodiments, a host, for example a human, is treated with an effective amount of an enantiomerically enriched mixture of enantiomers of a compound described herein, or a pharmaceutically acceptable salt, mixed salt, isotopic derivative, or prodrug thereof, wherein the percent of S enantiomer is greater than about 65 percent.

In certain embodiments, a host, for example a human, is treated with an effective amount of an enantiomerically enriched mixture of enantiomers of a compound described herein, or a pharmaceutically acceptable salt, mixed salt, isotopic derivative, or prodrug thereof, wherein the percent of S enantiomer is greater than about 60 percent.

In certain embodiments, a host, for example a human, is treated with an effective amount of an enantiomerically enriched mixture of enantiomers of a compound described herein, or a pharmaceutically acceptable salt, mixed salt, isotopic derivative, or prodrug thereof, wherein the percent of S enantiomer is greater than about 55 percent.

In certain embodiments, a host, for example a human, is treated with an effective amount of an enantiomerically enriched mixture of enantiomers of a compound described herein, or a pharmaceutically acceptable salt, mixed salt, isotopic derivative, or prodrug thereof, wherein the percent of S enantiomer is greater than about 55 or 60 percent.

In certain embodiments, a host, for example a human, is treated with an effective amount of an enantiomerically enriched mixture of enantiomers of a compound described herein, or a pharmaceutically acceptable salt or mixed salt, isotopic derivative, or prodrug thereof, wherein the percent of R enantiomer is greater than about 95 percent.

In certain embodiments, a host, for example a human, is treated with an effective amount of an enantiomerically enriched mixture of enantiomers of a compound described herein, or a pharmaceutically acceptable salt mixed salt, isotopic derivative, or prodrug thereof, wherein the percent of R enantiomer is greater than about 90 percent.

In certain embodiments, a host, for example a human, is treated with an effective amount of an enantiomerically enriched mixture of enantiomers of a compound described herein, or a pharmaceutically acceptable salt or mixed salt, isotopic derivative, or prodrug thereof, wherein the percent of R enantiomer is greater than about 85 percent.

In certain embodiments, a host, for example a human, is treated with an effective amount of an enantiomerically enriched mixture of enantiomers of a compound described herein, or a pharmaceutically acceptable salt or mixed salt, isotopic derivative, or prodrug thereof, wherein the percent of R enantiomer is greater than about 80 percent.

In certain embodiments, a host, for example a human, is treated with an effective amount of an enantiomerically enriched mixture of enantiomers of a compound described herein, or a pharmaceutically acceptable salt or mixed salt, isotopic derivative, or prodrug thereof, wherein the percent of R enantiomer is greater than about 75 percent.

In certain embodiments, a host, for example a human, is treated with an effective amount of an enantiomerically enriched mixture of enantiomers of a compound described herein, or a pharmaceutically acceptable salt, mixed salt, isotopic derivative, or prodrug thereof, wherein the percent of R enantiomer is greater than about 70 percent.

In certain embodiments, a host, for example a human, is treated with an effective amount of an enantiomerically enriched mixture of enantiomers of a compound described herein, or a pharmaceutically acceptable salt, mixed salt, isotopic derivative, or prodrug thereof, wherein the percent of R enantiomer is greater than about 65 percent.

In certain embodiments, a host, for example a human, is treated with an effective amount of an enantiomerically enriched mixture of enantiomers of a compound described herein, or a pharmaceutically acceptable salt, mixed salt, isotopic derivative, or prodrug thereof, wherein the percent of R enantiomer is greater than about 60 percent.

In certain embodiments, a host, for example a human, is treated with an effective amount of an enantiomerically enriched mixture of enantiomers of a compound described herein, or a pharmaceutically acceptable salt, mixed salt, isotopic derivative, or prodrug thereof, wherein the percent of R enantiomer is greater than about 55 percent.

In certain embodiments, a host, for example a human, is treated with an effective amount of an enantiomerically enriched mixture of enantiomers of a compound described herein, or a pharmaceutically acceptable salt, mixed salt, isotopic derivative, or prodrug thereof, wherein the percent of R enantiomer is greater than about 55 or 60 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of R enantiomer is greater than about 99 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of R enantiomer is greater than about 95 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of R enantiomer is greater than about 90 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of R enantiomer is greater than about 85 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of R enantiomer is greater than about 80 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of R enantiomer is greater than about 75 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of R enantiomer is greater than about 70 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of R enantiomer is greater than about 65 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of R enantiomer is greater than about 60 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of R enantiomer is greater than about 55 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of R enantiomer is greater than about 55 or 60 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of S enantiomer is greater than about 99 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of S enantiomer is greater than about 95 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of S enantiomer is greater than about 90 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of S enantiomer is greater than about 85 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of S enantiomer is greater than about 80 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of S enantiomer is greater than about 75 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of S enantiomer is greater than about 70 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of S enantiomer is greater than about 65 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of S enantiomer is greater than about 60 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of S enantiomer is greater than about 55 percent.

In certain embodiments, a host, for example a human, is treated with an effective amount of an enantiomerically enriched mixture of enantiomers of 5-MAPB, 6-MAPB, 5-MBPB, 6-MBPB, Bk-5-MAPB, Bk-6-MAPB, Bk-5-MBPB, or Bk-6-MBPB, or a pharmaceutically acceptable salt, mixed salt, isotopic derivative, or prodrug thereof, wherein the percent of S enantiomer is greater than about 55 or 60 percent.

The present invention also provides methods for modulating the CNS in a mammal in need thereof, including a human, by administering a pharmaceutically effective amount of a compound of the present invention, including S-5-MAPB, R-5-MAPB, S-6-MAPB, and/or R-6-MAPB or a pharmaceutically acceptable salt or mixed salt thereof.

In some embodiments, a method is provided for modulating the CNS in a mammal in need thereof, including a human, comprising administering a combination of an entactogenic compound and a dopamine release agent wherein the entactogenic compound is 5-MBPB and/or 6-MBPB or a pharmaceutically acceptable salt thereof.

In one embodiment, a method is provided to treat diseases or disorders linked to inadequate functioning of neurotransmission in the CNS comprising administering 5-MBPB and 6-MBPB or a pharmaceutically acceptable salt thereof in a host in need thereof.

In one embodiment, a method is provided to treat diseases or disorders linked to inadequate functioning of neurotransmission in the CNS comprising administering Bk-5-MAPB and Bk-6-MAPB or a pharmaceutically acceptable salt thereof in a host in need thereof.

In one embodiment, a method is provided to treat diseases or disorders linked to inadequate functioning of neurotransmission in the CNS comprising administering Bk-5-MBPB and Bk-6-MBPB or a pharmaceutically acceptable salt thereof in a host in need thereof.

In one embodiment, a method is provided to treat diseases or disorders linked to inadequate functioning of neurotransmission in the CNS comprising administering a combination of entactogenic compound and dopamine release agent or a pharmaceutically acceptable salt thereof in a host in need thereof.

This invention also provides the use S-5-MAPB, R-5-MAPB, S-6-MAPB, and/or R-6-MAPB or a pharmaceutically acceptable salt or composition to treat a maladaptive response to perceived psychological threats. In one embodiment, S-5-MAPB, R-5-MAPB, S-6-MAPB, and/or R-6-MAPB or a pharmaceutically acceptable salt or composition is administered in the context of psychotherapy. In one embodiment, S-5-MAPB, R-5-MAPB, S-6-MAPB, and/or R-6-MAPB or a pharmaceutically acceptable salt or composition is administered as a stand-alone treatment.

This invention also provides the administration of an effective amount of 5-MBPB and/or 6-MBPB or a pharmaceutically acceptable salt or composition to a host, typically a human, to treat a maladaptive response to perceived psychological threats. In one embodiment, 5-MBPB and/or 6-MBPB or a pharmaceutically acceptable salt or composition is administered in the context of psychotherapy. In one embodiment, 5-MBPB and/or 6-MBPB or a pharmaceutically acceptable salt or composition is administered as a stand-alone treatment.

This invention also provides the use Bk-5-MAPB and/or Bk-6-MAPB or a pharmaceutically acceptable salt or composition to treat a maladaptive response to perceived psychological threats. In one embodiment, Bk-5-MAPB and/or Bk-6-MAPB or a pharmaceutically acceptable salt or composition is administered in the context of psychotherapy. In one embodiment, Bk-5-MAPB and/or Bk-6-MAPB or a pharmaceutically acceptable salt or composition is administered as a stand-alone treatment.

This invention also provides the use Bk-5-MBPB and/or Bk-6-MBPB or a pharmaceutically acceptable salt or composition to treat a maladaptive response to perceived psychological threats. In one embodiment, Bk-5-MBPB and/or Bk-6-MBPB or a pharmaceutically acceptable salt or composition is administered in the context of psychotherapy. In one embodiment, Bk-5-MBPB and/or Bk-6-MBPB or a pharmaceutically acceptable salt or composition is administered as a stand-alone treatment.

### Non-Limiting Examples of Pharmacotherapeutic Use

Psychotherapy, cognitive enhancement, or life coaching conducted with the compositions and combinations described herein employed as an adjunct (hereafter, “pharmacotherapy”) is typically conducted in widely spaced sessions with one, two, or rarely three or more administrations of an entactogen per session. These sessions can be as frequent as weekly, but are more often approximately monthly or even less frequently. In most cases, a small number of pharmacotherapy sessions, on the order of one to three, is needed for the patient to experience significant clinical progress, as indicated, for example, by a reduction in signs and symptoms of mental distress, by improvement in functioning in some domain of life, by arrival at a satisfactory solution to some problem, or by increased feelings of closeness to and understanding of some other person. In some embodiments, the psychotherapy, cognitive enhancement, or life coaching is conducted with an effective amount of enantiomerically enriched S-5-MAPB, R-5-MAPB, S-6-MAPB, and/or R-6-MAPB or a pharmaceutically acceptable salt thereof. In some embodiments, the psychotherapy, cognitive enhancement, or life coaching is conducted with an effective amount of enantiomerically enriched Bk-5-MAPB and/or Bk-6-MAPB or a pharmaceutically acceptable salt thereof. Alternatively, the psychotherapy, cognitive enhancement, or life coaching is conducted with an effective amount of enantiomerically enriched Bk-5-MBPB and/or Bk-6-MBPB or a pharmaceutically acceptable salt thereof.

The following sections provide detailed examples of pharmacotherapy. While common procedures are described, these are intended as illustrative, non-limiting examples. It is anticipated that the prescribing physician and therapy team may wish to specify different procedures than those described here based on their clinical judgment concerning the needs of the patient.

The example methods of treatment can also be modified with very minor changes to treat multiple patients at once, including couples or families. Hence, “patient” should be understood to mean one or more individuals.

In some embodiments, the benefits of the current compositions are measured in animal assays using drug administration procedures to produce appropriate lags in drug concentrations. Such drug administration procedures can include timed injections and oral or gavage administration. Benefits detectable in animal assays include, but are not limited to, improved therapeutic effects, decreases in undesired effects, and improved ratios of therapeutic effects to undesired effects. Exemplary therapeutic effects can be measured in assays such as drug discrimination assays, social choice assays, and acute anxiety assays. Drug discrimination assays, in which animals are trained to distinguish MDMA from placebo, are summarized using the ED50, the dose at which 50% of animals generalize from MDMA to the novel composition with generalization defined as a predefined high (e.g. 80) percent of MDMA-appropriate lever presses (e.g., Baker, Behavioral Neurobiology of Psychedelic Drugs (2017): 201-219). Accordingly, the ED50 is herein interpreted as a measure of acute therapeutic effects. Social choice assays measure time an animal chooses to stay in proximity of another animal compared to time near an object (e.g., Heifets & Malenka, Cell, 166(2), 269-272; Heifets et al. Science translational medicine 11.522 (2019): eaaw6435) and are herein considered a measure of decreased social anxiety. Undesired effects of the current compositions can be measured with well-known rodent assays for anxiety (e.g., elevated plus maze) as well as assays of cardiovascular effects and physiological effects (e.g., body temperature, hepatotoxicity) as can be collected in free-ranging animals using implanted telemetry equipment and bioassay. Another undesired effect is decreased motivation up to the approximately 72 hours after drug administration as can be measured as the breaking point in an operant progressive ratio task in which the animal can receive a reward (e.g., a sucrose pellet or solution) by pressing a lever an increasing number of times (e.g. Plaza-Zabala et al. Psychopharmacology 208.4 (2010): 563-573). Additional undesired effects include behavioral changes such as locomotor stimulation or suppression (e.g. increases or decreases in distance travelled) and stereotypy, as can be measured with automated systems such as digital video. A non-limiting example of a benefit of the current compositions is thus a lowered ED50 from administering R-BK-5-MAPB before S-BK-5-MAPB without increasing acute temperature or blood pressure in comparison to S-BK-5-MAPB given alone.

Use of a Composition or Combination of the Present Invention in Conjunction with Conventional Psychotherapy or Coaching

In one embodiment, the use of a composition or combination of the present invention as pharmacotherapy is integrated into the patient's ongoing psychotherapy or coaching (hereafter abbreviated as “psychotherapy”). If a patient in need of the pharmacotherapy is not in ongoing psychotherapy, then psychotherapy may be initiated and the pharmacotherapy added later, after the prescribing physician and treating psychotherapist, physician, coach, member of the clergy, or other similar professional or someone acting under the supervision of such a professional (hereafter, “therapist”) agree that the pharmacotherapy is indicated and that there have been sufficient meetings between the patient and therapist to establish an effective therapeutic alliance.

If the patient is not experienced with the pharmacotherapy, a conversation typically occurs in which the therapist or other members of the therapy team addresses the patient's questions and concerns about the medicine and familiarizes the patient with the logistics of pharmacotherapy-assisted session. The therapist describes the kinds of experience that can be expected during the pharmacotherapy session. Optionally, parts of this conversation employ written, recorded, or interactive digital explanations, as might be used in the informed consent process in a clinical trial. The therapist may additionally make commitments to support the participant's healthcare and wellness process. In turn, the patient may be asked to make commitments of their own (such as not to hurt themselves or others and to abstain from contraindicated medicines or drugs for an adequate period before and after the pharmacotherapy).

The composition or combination of the invention (or alternately herein for convenience, the “medicine”) is administered shortly before or during a scheduled psychotherapy session, with timing optionally selected so that therapeutic effects begin by the time the psychotherapy session begins. Either shortly before or after administration of the medicine, it is common for the therapist to provide some reminder of their mutual commitments and expected events during the session.

The psychotherapy session is carried out by the therapist, who, optionally, may be remote and in communication with the patient using a communication means suitable for telehealth or telemedicine, such as a phone, video, or other remote two-way communication method. Optionally, video or other monitoring of the patient's response or behavior is used to document or measure the session. The therapist uses their clinical judgment and available data to adjust the session to the needs of the patient. Many therapists view their responsibility as being to facilitate rather than direct the patient's experience. This may sometimes involve silent empathic listening, while other times it may include more active support to help the patient arrive at new perspectives on their life.

It is anticipated that the therapeutic effects of the medicine will allow the patient to make more rapid therapeutic progress than would normally be possible. These effects include decreased neuroticism and increased feelings of authenticity. Patients are often able to calmly contemplate actual or possible experiences that would normally be upsetting or even overwhelming. This can facilitate decision making and creativity in addition to mental wellness.

Optionally, the prescribing physician may allow a second or even third administration of the medicine or another psychotherapeutic agent in order to extend the therapeutic effects. Optionally, a pharmaceutical preparation with modified release is employed to make this unnecessary.

Because the duration of the scheduled psychotherapy session may be shorter than the therapeutic effects of the medicine, the therapist may suggest to the patient activities to support further psychotherapeutic progress after the psychotherapy session has ended. Alternatively, the therapist may continue to work with the patient until the therapeutic effects of the medicine have become clinically minimal.

In a subsequent non-pharmacological psychotherapy session, the therapist and patient will typically discuss the patient's experiences from the pharmacotherapy session and the therapist will often aid the patient in recalling the therapeutic effects and help them to incorporate the experiences into their everyday lives.

Pharmacotherapy sessions may be repeated as needed, based on the judgment of the treating physician and therapy team regarding the needs of the patient.

### Use of a Composition or Combination of the Present Invention Outside of Conventional Psychotherapy

In one embodiment, a composition or combination of the present invention is administered outside of a conventional psychotherapy. This example method is a broader, more flexible approach to pharmacotherapy that is not centered on supervision by a therapist. These pharmacotherapy sessions can take place in many different quiet and safe settings, including the patient's home. The setting is typically chosen to offer a quiet setting, with minimal disruptions, where the patient feels psychologically safe and emotionally relaxed. The setting may be the patient's home but may alternatively be a clinic, retreat center, or hotel room.

In one alternative embodiment, the medicine is taken by the patient regularly to maintain therapeutic concentrations of the active compound in the blood. In another alternative embodiment, the medicine is taken, as needed, for defined psychotherapy sessions.

Optionally, a checklist may be followed to prepare the immediate environment to minimize distractions and maximize therapeutic or decision-making benefits. This checklist can include items such as silencing phones and other communications devices, cleaning and tidying the environment, preparing light refreshments, preparing playlists of appropriate music, and pre-arranging end-of-session transportation if the patient is not undergoing pharmacotherapy at home.

Before the pharmacotherapy session, there may be an initial determination of the therapeutic or other life-related goals (for example, decision-making, increasing creativity, or simply appreciation of life) that will be a focus of the session. These goals can optionally be determined in advance with support from a therapist.

Optionally, the therapist may help the patient select stimuli, such as photographs, videos, augmented or virtual reality scenes, or small objects such as personal possessions, that will help focus the patient's attention on the goals of the session or on the patient's broader life journey. As examples that are intended to be illustrative and not restrictive, these stimuli can include photographs of the patient from when they were young, which can increase self-compassion, or can include stimuli relating to traumatic events or phobias experienced by the patient, which can help the patient reevaluate and change their response to such stimuli. Optionally, the patient selects these stimuli without assistance (e.g., without the involvement of the therapist) or does not employ any stimuli. Optionally, stimuli are selected in real time by the therapist or an algorithm based on the events of the session with the goal of maximizing benefits to the patient.

If the patient is not experienced with the pharmacotherapy, a conversation occurs in which the therapist addresses the patient's questions and concerns about the medicine and familiarizes the patient with the logistics of a pharmacotherapy-assisted session. The therapist describes the kinds of experience that can be expected during the pharmacotherapy-assisted session. Optionally, parts of this conversation employ written, recorded, or interactive digital explanations, as might be used in the informed consent process in a clinical trial. The therapist may additionally make commitments to support the participant's healthcare and wellness process. In turn, the patient may be asked to make commitments of their own (such as not to hurt themselves or others and to abstain from contraindicated medicines or drugs for an adequate period before and after the pharmacotherapy).

Selected session goals and any commitments or other agreements regarding conduct between the patient and therapy team are reviewed immediately before administration of the medicine. Depending on the pharmaceutical preparation and route of administration, the therapeutic effects of the medicine usually begin within one hour. Typical therapeutic effects include decreased neuroticism and increased feelings of authenticity. Patients are often able to calmly contemplate experiences or possible experiences that would normally be upsetting or even overwhelming. This can facilitate decision making and creativity in addition to mental wellness.

Optionally, sleep shades and earphones with music or soothing noise may be used to reduce distractions from the environment. Optionally, a virtual reality or immersive reality system may be used to provide stimuli that support the therapeutic process. Optionally, these stimuli are preselected; optionally, they are selected in real time by a person or an algorithm based on events in the session with the goal of maximizing benefits to the patient. Optionally, a therapist or other person well-known to the patient is present or available nearby or via phone, video, or other communication method in case the patient wishes to talk, however the patient may optionally undergo a session without the assistance of a therapist. Optionally, the patient may write or create artwork relevant to the selected session goals. Optionally, the patient may practice stretches or other beneficial body movements, such as yoga (“movement activity”).

Optionally, in other embodiments the patient may practice movement activity that includes more vigorous body movements, such as dance or other aerobic activity. Movement activity also may make use of exercise equipment such as a treadmill or bicycle.

In some additional embodiments, the patient may be presented with music, video, auditory messages, or other perceptual stimuli. Optionally, these stimuli may be adjusted based on the movements or other measurable aspects of the patient. Such adjustment may be done by the therapist with or without the aid of a computer, or by a computer alone in response to said patient aspects, including by an algorithm or artificial intelligence, and “computer” broadly meaning any electronic tool suitable for such purposes, whether worn or attached to a patient (e.g., watches, fitness trackers, “wearables,” and other personal devices; biosensors or medical sensors; medical devices), whether directly coupled or wired to a patient or wirelessly connected (and including desktop, laptop, and notebook computers; tablets, smartphones, and other mobile devices; and the like), and whether within the therapy room or remote (e.g., cloud-based systems).

For example, measurable aspects of a patient (e.g., facial expression, eye movements, respiration rate, pulse rate, skin color change, patient voice quality or content, patient responses to questions) from these tools may be individually transformed into scores on standardized scales by subtracting a typical value and then multiplying by a constant and these scores may be further multiplied by constants and added together to create an overall score that can optionally be transformed by multiplication with a link function, such as the logit function, to create an overall score. This score may be used to select or adjust stimuli such as selecting music with higher or lower beats-per-minute or with faster or slower notes, selecting images, audio, or videos with different emotionality or autobiographical meaning, or selecting activities for the patient to engage in (such as specific movements, journaling prompts, or meditation mantras).

It should be readily appreciated that a patient can participate in numerous therapeutically beneficial activities, where such participation follows or is in conjunction with the administration of a compound or composition of the invention, including writing about a preselected topic, engaging in yoga or other movement activity, meditating, creating art, viewing of photographs or videos or emotionally evocative objects, using a virtual reality or augmented reality system, talking with a person, and thinking about a preselected problem or topic, and it should be understood that such participation can occur with or without the participation or guidance of a therapist.

Optionally, the prescribing physician may allow a second or even third administration of the medicine or another psychotherapeutic agent in order to extend the therapeutic effects. Optionally, a pharmaceutical preparation with modified release is employed to make this unnecessary.

The patient typically remains in the immediate environment until the acute therapeutic effects of the medicine are clinically minimal, usually within eight hours. After this point, the session is considered finished.

The treatment plan will often include a follow-up session with a therapist. This follow-up session occurs after the pharmacotherapy session has ended, often the next day but sometimes several days later. In this session, the patient discusses their experiences from the pharmacotherapy session with the therapist, who can aid them in recalling the therapeutic effects and help them to incorporate the experiences into their everyday lives.

Pharmacotherapy sessions may be repeated as needed, based on the judgment of the treating physician and therapy team regarding the needs of the patient.

### Entactogenic Compounds and Dopamine Release Agents

In certain embodiments the entactogenic compound or dopamine release agent for use in a combination therapy is an enantiomerically enriched mixture.

An enantiomerically enriched mixture is a mixture that contains one enantiomer in a greater amount than the other. An enantiomerically enriched mixture of an S-enantiomer contains at least 55% of the S-enantiomer, and, typically at least about 60%, 65%, 70%, 75%, 80%, 85%, 90%, or 95% or more of the S-enantiomer. An enantiomerically enriched mixture of an R-enantiomer contains at least 55% of the R-enantiomer, and typically at least about 60%, 65%, 70%, 75%, 80%, 85%, 90% or 95% of the R-enantiomer. The specific ratio of S or R enantiomer can be selected for the need of the patient according to the health care specialist to balance the desired effect.

The term enantiomerically enriched mixture as used in this application does not include a racemic mixture and does not include a pure isomer or substantially pure isomer. Notwithstanding, it should be understood that any compound described herein in enantiomerically enriched form can be used as a substantially pure isomer if it achieves the goal of any of the specifically itemized methods of treatment described herein, including but not limited to 5-MAPB, 6-MAPB, 5-MBPB, 6-MBPB, 5-Bk-MAPB, 6-Bk-MAPB, Bk-5-MBPB or Bk-6-MBPB.

The chiral carbon typically referred to in this application is the carbon alpha to the amine in the phenylethylamine motif. Of course, the compounds can have additional chiral centers that result in diastereomers. Notwithstanding, in the present application, the primary chiral carbon referred to in the term “enantiomerically enriched” is that carbon alpha to the amine in the provided structures.

In one aspect of the invention, compounds are provided comprising enantiomerically enriched or enantiomerically substantially pure R-5-MAPB, S-5-MAPB, R-6-MAPB, or R-6-MAPB or a pharmaceutically acceptable salt or mixed salt thereof. In one embodiment, a pharmaceutical composition is provided that comprises an enantiomerically-enriched mixture of the R- or S-enantiomer of 5-MAPB or 6-MAPB:

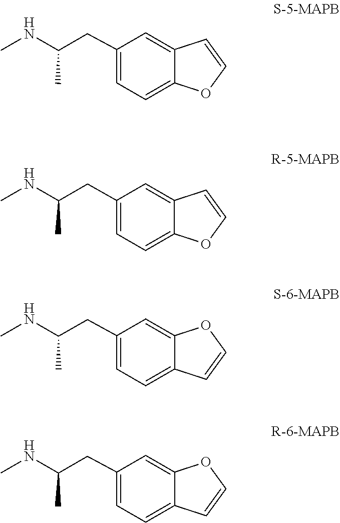

In certain embodiments the pharmaceutical composition comprises, includes, consists essentially of or consists of at least two active agents, and one or more tableting excipients, wherein at least one active agent is formulated for an immediate release and at least one agent is formulated for a delayed release.

In certain embodiments the pharmaceutical composition comprises, includes, consists essentially of, or consists of amphetamine and enantiomerically enriched S-MDMA active agents wherein one active agent is formulated for immediate release, and the other agent is formulated for a delayed release.

In certain embodiments the pharmaceutical composition comprises, includes, consists essentially of, or consists of enantiomerically enriched S-MDMA and enantiomerically enriched R-MDMA wherein one active agent is formulated for immediate release, and the other agent is formulated for a delayed release.

In certain embodiments the pharmaceutical composition comprises, includes, consists essentially of, or consists of amphetamine and enantiomerically enriched S-5-MAPB active agents wherein one active agent is formulated for immediate release, and the other agent is formulated for a delayed release.

In certain embodiments the pharmaceutical composition comprises, includes, consists essentially of, or consists of R-BK-MDMA and S-BK-MDMA active agents wherein one active agent is formulated for immediate release, and the other agent is formulated for a delayed release.

In certain embodiments the pharmaceutical composition comprises, includes, consists essentially of, or consists of S-5-APB and R-5-APB active agents wherein one active agent is formulated for immediate release, and the other agent is formulated for a delayed release.

In certain embodiments the pharmaceutical composition comprises, includes, consists essentially of, or consists of S-6-APB and R-6-APB active agents wherein one active agent is formulated for immediate release, and the other agent is formulated for a delayed release.

In certain embodiments the pharmaceutical composition comprises, includes, consists essentially of, or consists of S-BK-5-MAPB and R-BK-5-MAPB active agents wherein one active agent is formulated for immediate release, and the other agent is formulated for a delayed release.

In certain embodiments the pharmaceutical composition comprises, includes, consists essentially of, or consists of S-6-MBPB and R-6-MBPB active agents wherein one active agent is formulated for immediate release, and the other agent is formulated for a delayed release.

In certain embodiments the pharmaceutical composition comprises, includes, consists essentially of, or consists of S-5-MBPB and R-5-MBPB active agents wherein one active agent is formulated for immediate release, and the other agent is formulated for a delayed release.

In certain embodiments the pharmaceutical composition includes microcrystalline cellulose, croscarmellose sodium, and magnesium stearate.

In certain specific embodiments the pharmaceutical composition comprises a third active agent used as a selective serotonin reuptake inhibitor (SSRI) or a dual serotonin-norepinephrine reuptake inhibitor (SNRI) and a blocking agent, wherein the third active agent is formulated for delayed release following the delayed release of the second agent. Non-limiting examples of the third active agent include milnacipran, citalopram, duloxetine, venlafaxine, desvenlafaxine, milnacipran, and levomilnacipran.

In certain embodiments a fourth active agent is used wherein the fourth active agent is a compound described herein for example an entactogen, dopamine releaser, selective serotonin reuptake inhibitor, or dual serotonin-norepinephrine reuptake inhibitor.

In certain embodiments, isolated enantiomers of the compounds of the present invention show improved binding at the desired receptors and transporters relevant to the goal of treatment for the mental disorder or for mental enhancement.

It is useful to have an S- or R-enantiomerically enriched mixture of these entactogenic compounds that is not a racemic mixture. In certain embodiments the enantiomerically enriched mixtures that have a greater amount of the S-enantiomer 5-MAPB or 6-MAPB maximize serotonin-receptor-dependent therapeutic effects, whereas the enantiomerically enriched R-enantiomer of 5-MAPB or 6-MAPB maximize nicotinic-receptor-dependent therapeutic effects. Therefore, one aspect of the present invention is a balanced mixture of S-5-MAPB and R-5-MAPB or a balanced mixture of S-6-MAPB and R-6-MAPB that achieves a predetermined combination of serotonin-receptor-dependent therapeutic effects and nicotinic-receptor-dependent or dopaminergic therapeutic effects. The effect can be modulated as desired for optimal therapeutic effect.

Accordingly, in one embodiment, an enantiomerically enriched mixture of S-5-MAPB or an enantiomerically enriched mixture of S-6-MAPB maximize serotonin-receptor-dependent therapeutic effects and minimize unwanted nicotinic effects or dopaminergic effects when administered to a host in need thereof, for example a mammal, including a human.

In another embodiment, an enantiomerically enriched mixture of R-5-MAPB or an enantiomerically enriched mixture of R-6-MAPB maximize nicotinic-receptor-dependent or dopaminergic-receptor dependent therapeutic effects while minimizing unwanted effects, when administered to a host in need thereof, including a mammal, for example, a human.

Non-limiting examples of unwanted effects that can be minimized by carefully selecting the balance of enantiomers include hallucinogenic effects, psychoactive effects (such as excess stimulation or sedation), physiological effects (such as transient hypertension or appetite suppression), toxic effects (such as to the brain or liver), effects contributing to abuse liability (such as euphoria or dopamine release), and/or other side effects.

In certain embodiments the enantiomerically enriched mixtures of 5-MAPB that are non-racemic have a relatively greater amount of some therapeutic effects (such as emotional openness) while having lesser effects associated with abuse liability (such as perceptible ‘good drug effects’ which can lead to abuse versus openness, which leads to more tranquility and peace). Therefore, one aspect of the present invention is a balanced mixture of S-5-MAPB and R-5-MAPB or a balanced mixture of S-6-MAPB and R-6-MAPB that achieves a predetermined combination of emotional therapeutic effects and perceptible mood effects. The effect can be modulated as desired for optimal therapeutic effect.

Accordingly, in one embodiment, an enantiomerically enriched mixture of S-5-MAPB or an enantiomerically enriched mixture of S-6-MAPB balances emotional openness and perceptible mood effects when administered to a host in need thereof, for example a mammal, including a human.

In certain embodiments, it is preferred to have an S- or R-enantiomerically enriched mixture. Enantiomerically enriched mixtures that have a greater amount of the R-enantiomer of 5-MAPB or 6-MAPB maximize nicotinic-receptor-dependent therapeutic effects and that enantiomerically enriched mixtures that have a greater amount of the S-enantiomer 5-MAPB or 6-MAPB maximize serotonin-receptor-dependent therapeutic effects. Therefore, one aspect of the present invention is a balanced mixture of S-5-MAPB and R-5-MAPB or a balanced mixture of S-6-MAPB and R-6-MAPB that achieves a predetermined combination of serotonin-receptor-dependent therapeutic effects and nicotinic-receptor-dependent therapeutic effects.

Accordingly, in one embodiment, an enantiomerically enriched mixture of S-5-MAPB or an enantiomerically enriched mixture of S-6-MAPB maximize serotonin-receptor-dependent therapeutic effects and minimized unwanted nicotinic effects when administered to a host in need thereof, for example a mammal, including a human.

In another embodiment, an enantiomerically enriched mixture of R-5-MAPB or an enantiomerically enriched mixture of R-6-MAPB maximize nicotinic-receptor-dependent therapeutic effects while minimizing unwanted effects, when administered to a host in need thereof, including a mammal, for example, a human.

The present invention also provides new medical uses for combinations described herein by administering an effective amount to a patient such as a human to treat a CNS disorder including but not limited to, the treatment of depression, dysthymia, anxiety, generalized anxiety, social anxiety, panic, adjustment disorders, feeding and eating disorders, binge behaviors, body dysmorphic syndromes, addiction, drug abuse or dependence disorders, disruptive behavior disorders impulse control disorders, gaming disorders, gambling disorders, memory loss, dementia of aging, attention deficit hyperactivity disorder, personality disorders, attachment disorders, autism or dissociative disorders or any other disorder described herein, including in the Background.

In certain embodiments the entactogenic compounds for use in the current invention are direct 5-HT_{1B} agonists. Very few substances are known that are 5-HT_{1B} agonists and also 5-HT releasers and of those, some show significant toxicities. For example, m-chlorophenylpiperazine (mCPP) is one example but is anxiogenic and induces headaches, limiting any clinical use. MDMA itself does not bind to the 5-HT_{1B} (Ray. 2010. PloS one, 5(2), e9019). 5-HT_{1B} agonism is noteworthy because indirect stimulation of these receptors, secondary to elevated extracellular serotonin, has been hypothesized to be required for the prosocial effects of MDMA (Heifets et al. 2019. Science translational medicine, 11(522)), while other aspects of entactogen effects have been attributed to monoamine release (e.g., Luethi & Liechti. 2020. Archives of Toxicology, 94(4), 1085-1133). Thus, the unique ratios of 5-HT_{1B} stimulation and monoamine release displayed by the disclosed compounds enable different profiles of therapeutic effects that cannot be achieved by MDMA or other known entactogens.

In certain embodiments the compound for use in the present invention shows a 5-HT selectivity pattern that is important to therapeutic use. Various subtypes of 5-HT receptor can induce different felt experiences on a patient. Agonism of the 5-HT_{2A} receptor can cause feelings of fear and hallucinations, but agonism of 5-HT_{1B} is believed to be tied to the pro-social effects of entactogens. Various subtypes of 5-HT receptor can also contribute to different toxicity risks for a patient. Administration of MDMA and other serotonergic drugs is associated with elevated acute risk of hyponatremia. It is known that stimulation of 5-HT₂ receptors is an important trigger of release of antidiuretic hormone (Iovino et a. Current pharmaceutical design 18, no. 30 (2012): 4714-4724).

The enantiomeric compositions of the present invention can be selected to be poor agonists of 5-HT₂A, but exhibit activity toward 5-HT_{1B}. For example, as described in the non-limiting illustrative Example 6, the majority of the compounds do not exhibit 5-HT_{2A} agonist activity but do exhibit 5-HT_{1B} agonist activity in the range of about 5 to 0.0005 μM, or 3 to 0.10 μM. Importantly, 5-HT_{1B} agonist activity effect occurs through direct action on the receptor, rather than as an indirect consequence of serotonin release. This is an unexpected discovery because this property has not been observed in an entactogen, including MDMA, before. In one embodiment, the selectivity toward the 5-HT_{1B} receptor over 5-HT_{2A} receptor allows for a more relaxed and therapeutically productive experience for the patient undergoing treatment with a compound of the present invention.

The unique ratios of 5-HT_{1B} stimulation and 5-HT release displayed by the disclosed compounds enable different profiles of therapeutic effects and side effects that may not be achieved by MDMA or other known entactogens. An undesirable effect of releasing 5-HT can be hyponatremia or loss of appetite. Drugs such as d-fenfluramine that release 5-HT by interacting with SERT and thereby increase agonism of all serotonin receptors have been used as anorectics. Similarly, MDMA is known to acutely suppress appetite (see, e.g., Vollenweider et al. Neuropsychopharmacology 19, no. 4 (1998): 241-251.).

In another embodiment, therefore, the selectivity toward the 5-HT_{1B} receptor over SERT-mediated 5-HT release allows for a therapeutically productive experience for the patient undergoing treatment with a compound of the present invention with fewer other side effects from serotonin release, such as loss of appetite or risk of hyponatremia.

The present invention also uses compounds with beneficial selectivity profiles for neurotransmitter transporters. The balance of weakly activating NET (to reduce cardiovascular toxicity risk) and having a relatively low DAT to SERT ratio (to increase therapeutic effect relative to addictive liability) is a desirable feature of an entactogenic therapy displayed by the compounds and compositions of the present invention.

In certain aspects the entactogenic compound or dopamine releasing agent is “tuned” by administering an effective amount to a host such as a human, in need thereof, in a composition of a substantially pure enantiomer (or diastereomer, where relevant), or alternatively, an enantiomerically enriched composition that has an abundance of one enantiomer over the other. In this way, as described above, the enantiomeric forms act differently from each other on various 5-HT receptors, dopamine receptors, nicotinic acetylcholine receptors, and norepinephrine receptors, producing variable effects, and that those effects can be selected for based on desired outcome for the patient.

In certain embodiments, any of the mixture of the present invention is administered to a patient in an effective amount in conjunction with psychotherapy, cognitive enhancement, or life coaching (pharmacotherapy), or as part of routine medical therapy.

The compounds may be provided in a composition that is enantiomerically enriched, such as a mixture of enantiomers in which one enantiomer is present in excess, in particular to the extent of 60% or more, 70% or more, 75% or more, 80% or more, 90% or more, 95% or more, or 98% or more, including 100%.

In certain embodiments the combination of the present invention includes a compound selected from:

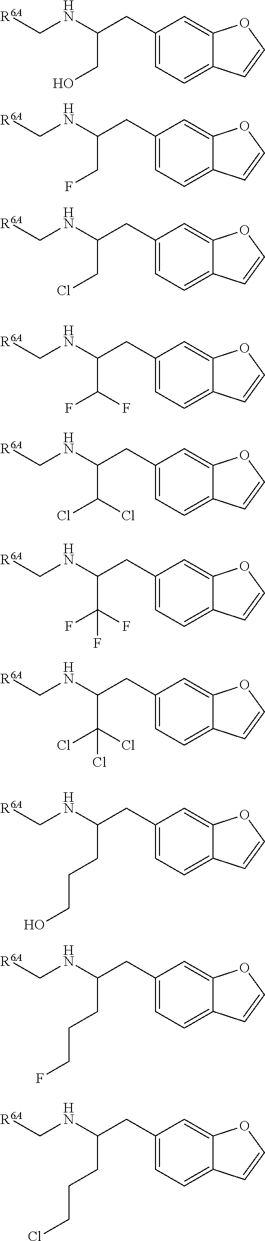

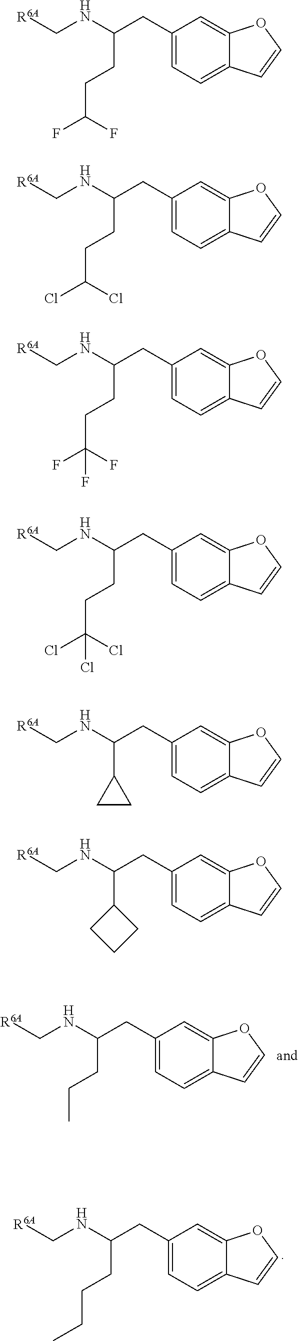

In certain embodiments the combination of the present invention includes a compound selected from:

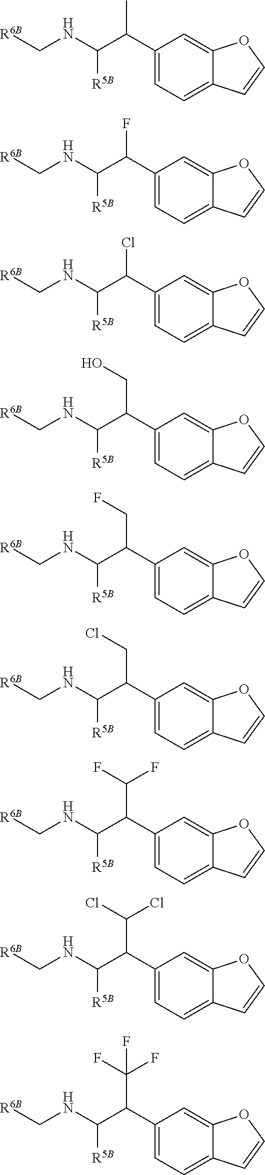

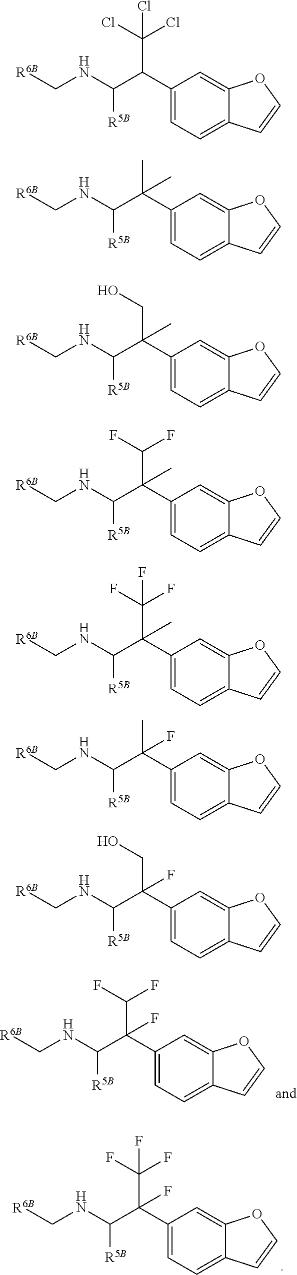

In certain embodiments the combination of the present invention includes a compound selected from:

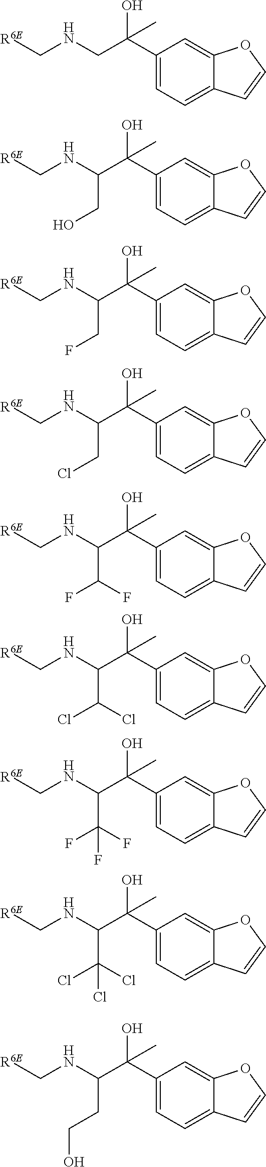

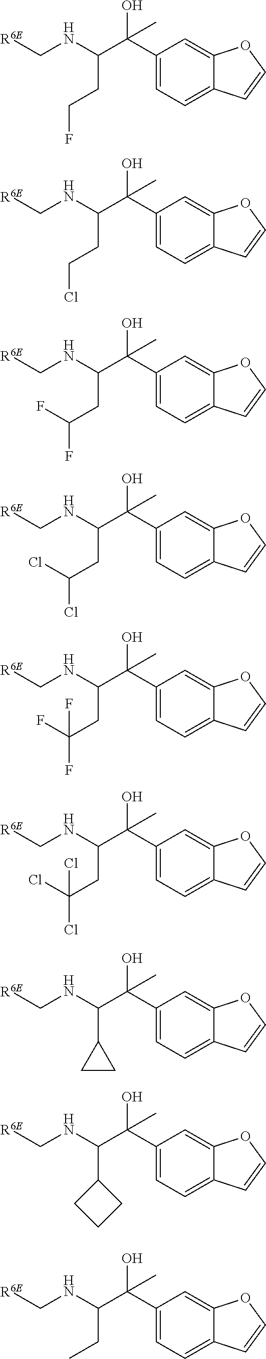

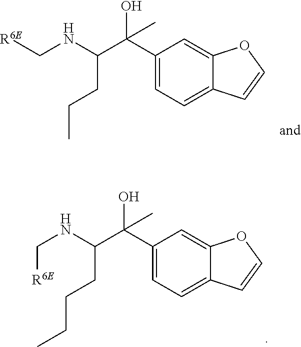

In certain embodiments the combination of the present invention includes a compound selected from:

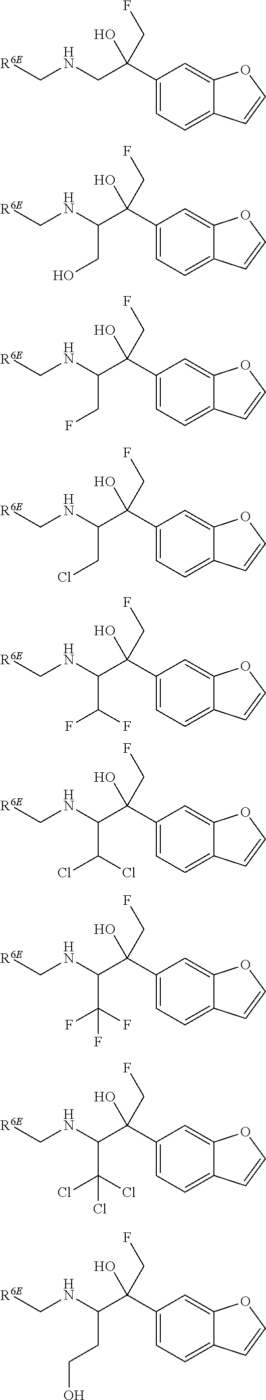

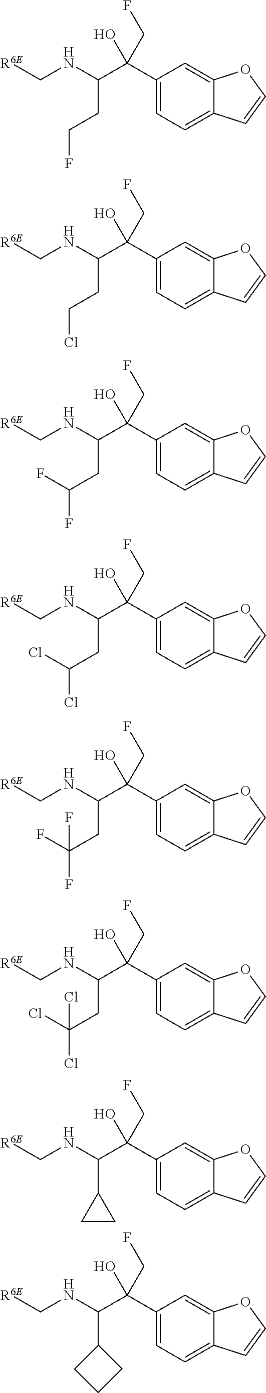

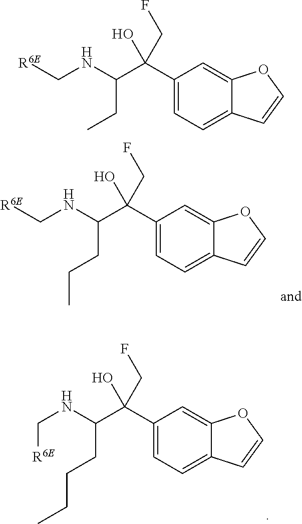

In certain embodiments the combination of the present invention includes a compound selected from:

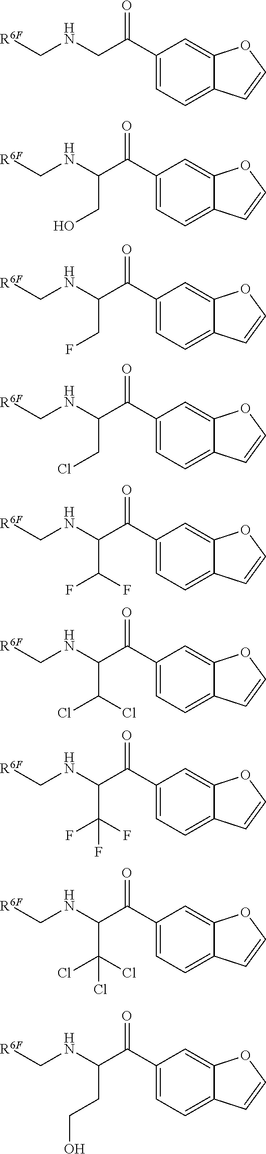

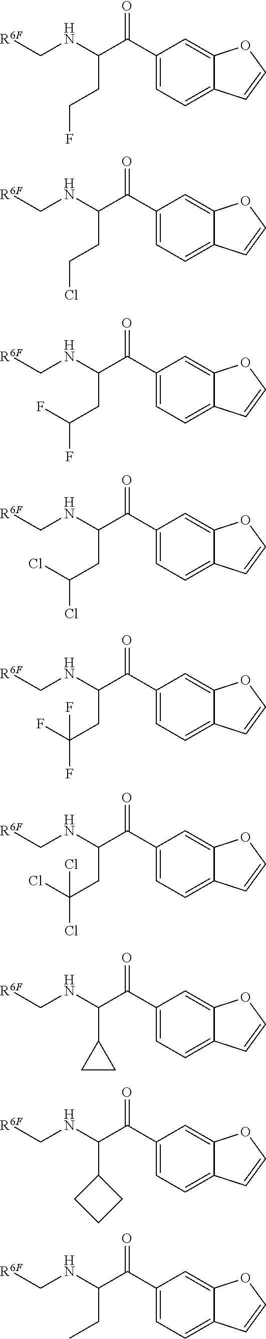

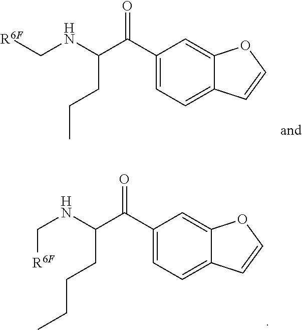

In certain embodiments the combination of the present invention includes a compound selected from:

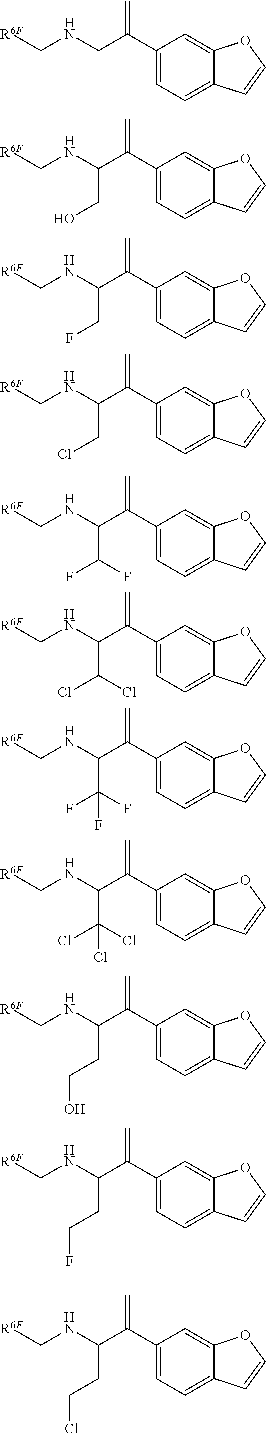

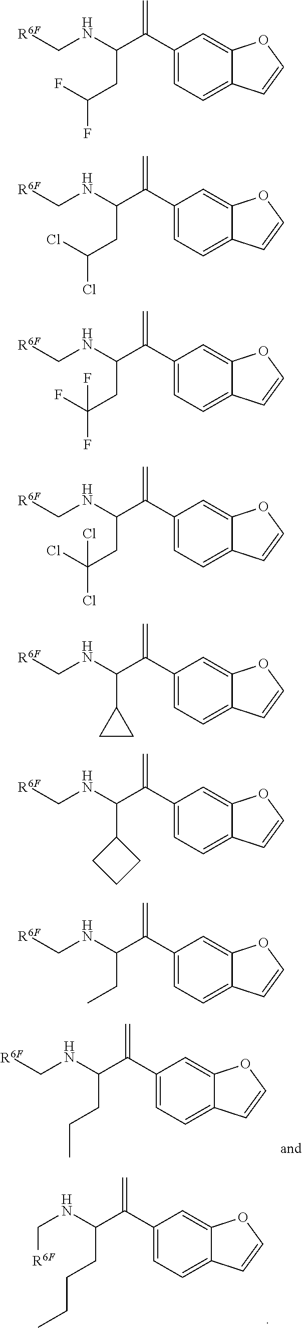

In certain embodiments the combination of the present invention includes a compound selected from:

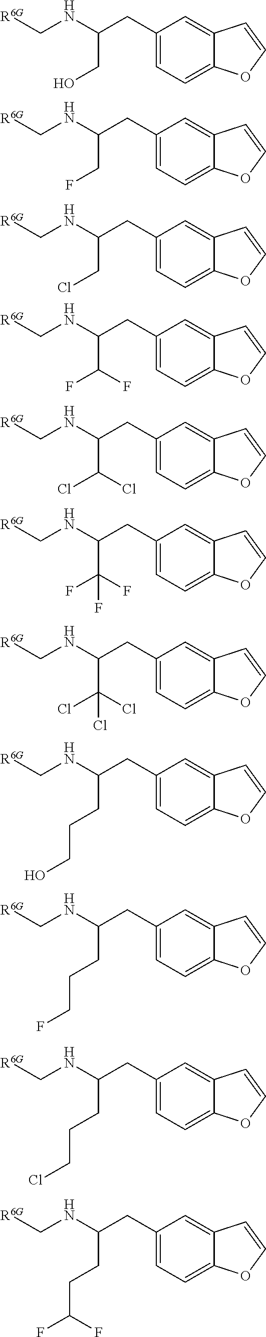

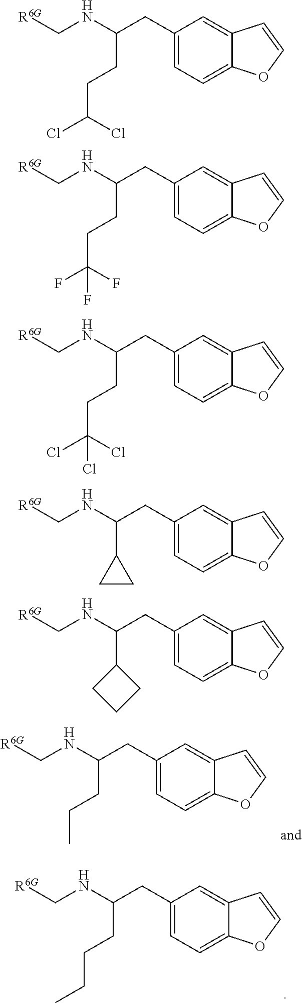

In certain embodiments the combination of the present invention includes a compound selected from:

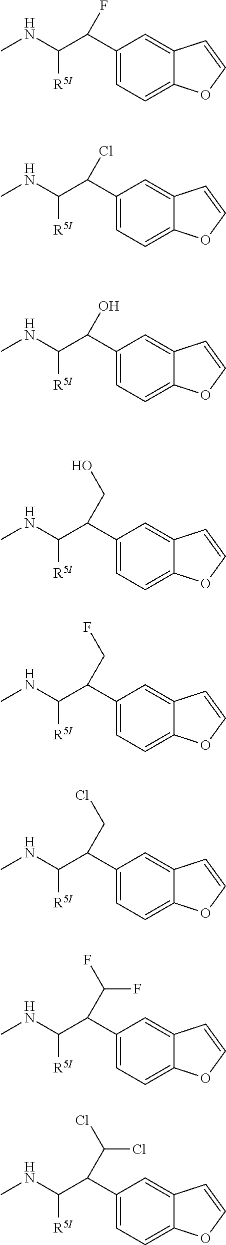

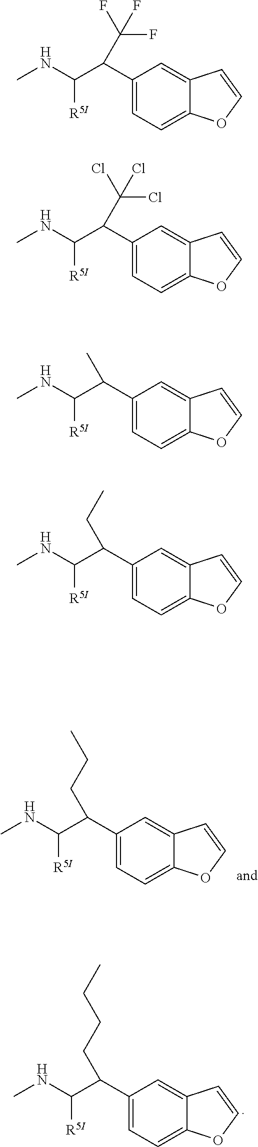

In certain embodiments the combination of the present invention includes a compound selected from:

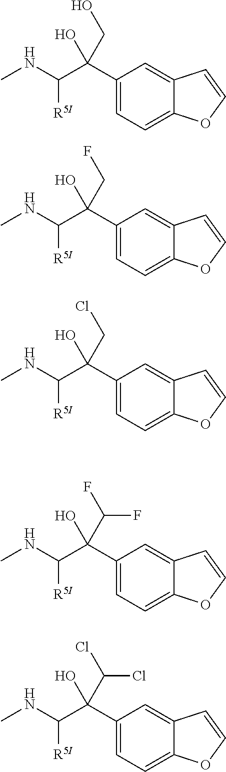

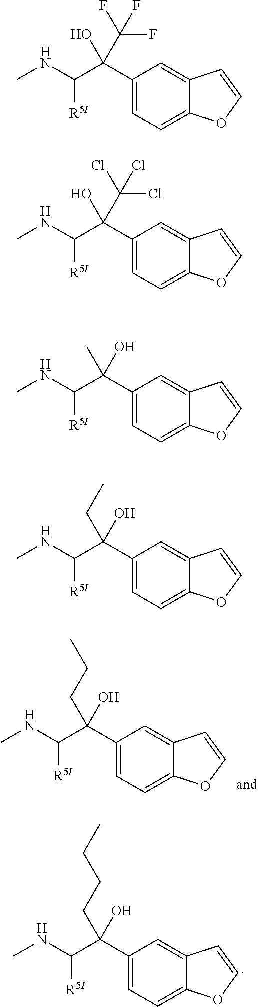

In certain embodiments the combination of the present invention includes a compound

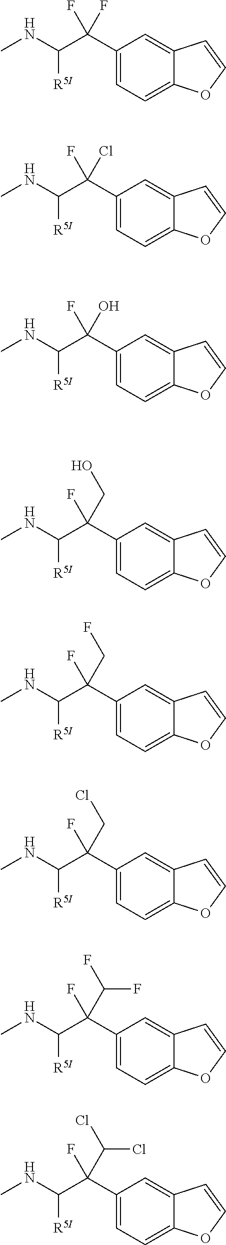

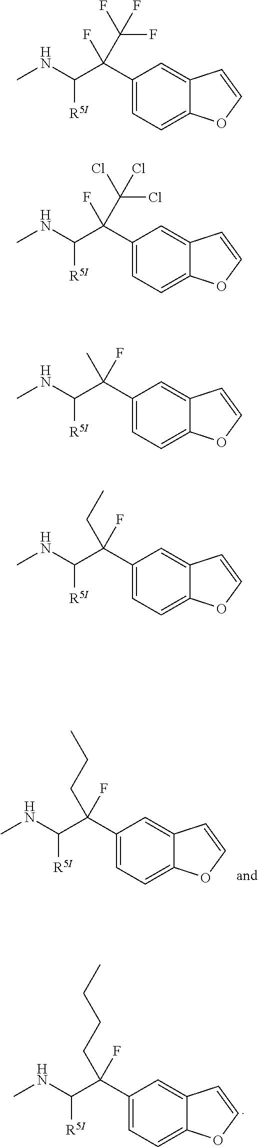

In certain embodiments the combination of the present invention includes a compound selected from:

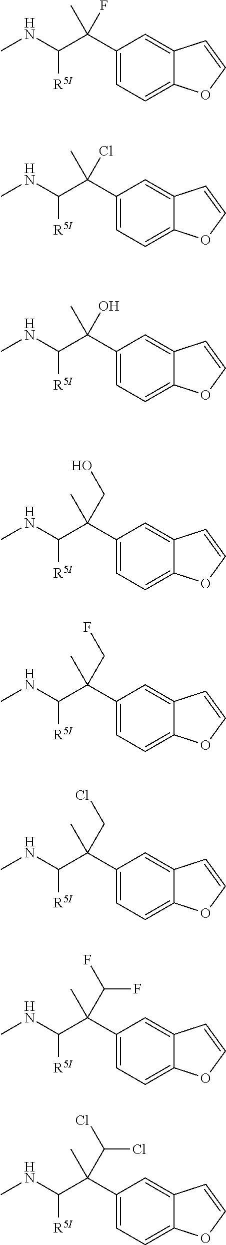

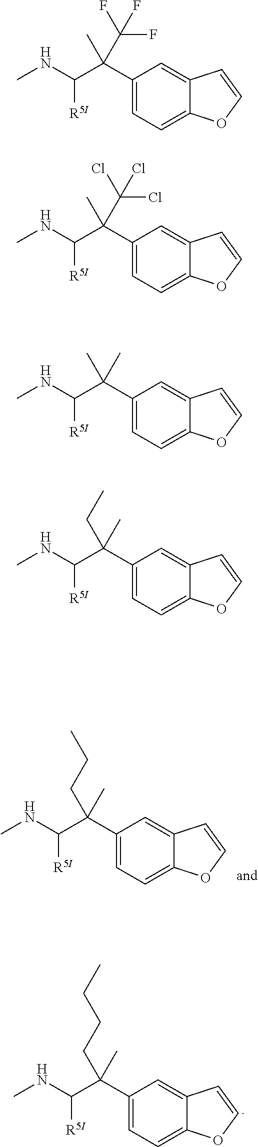

In certain embodiments the combination of the present invention includes a compound selected from:

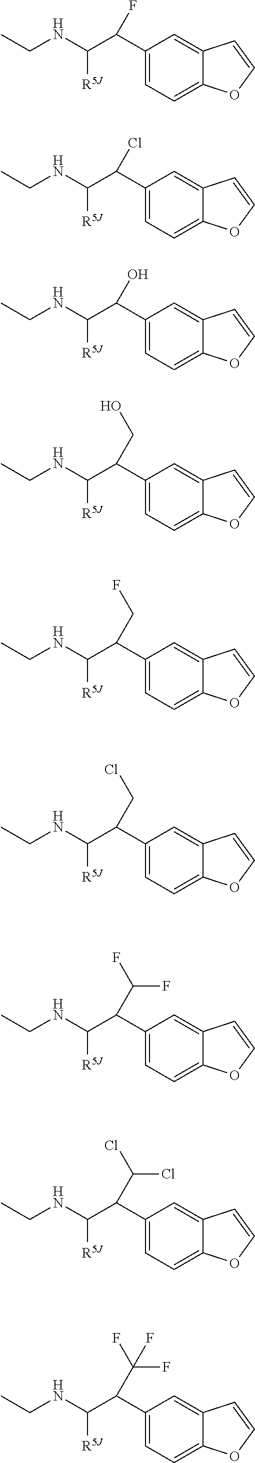

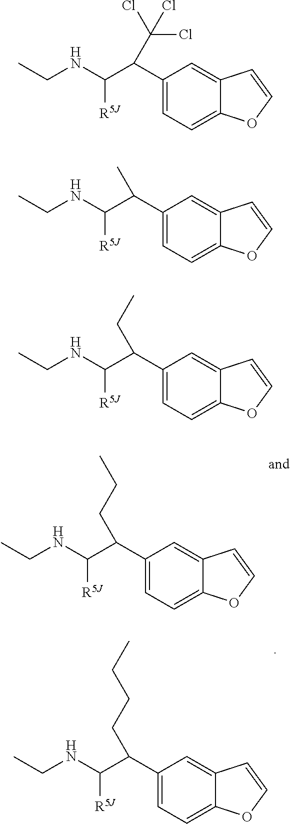

In certain embodiments the combination of the present invention includes a compound selected from:

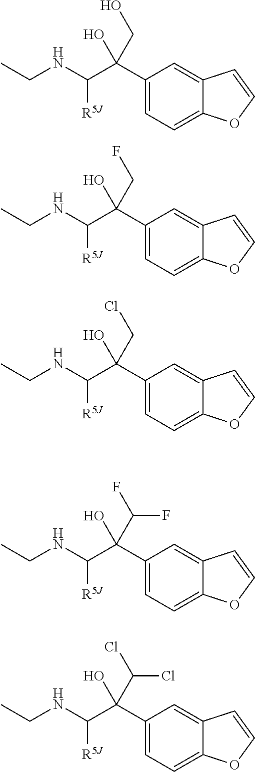

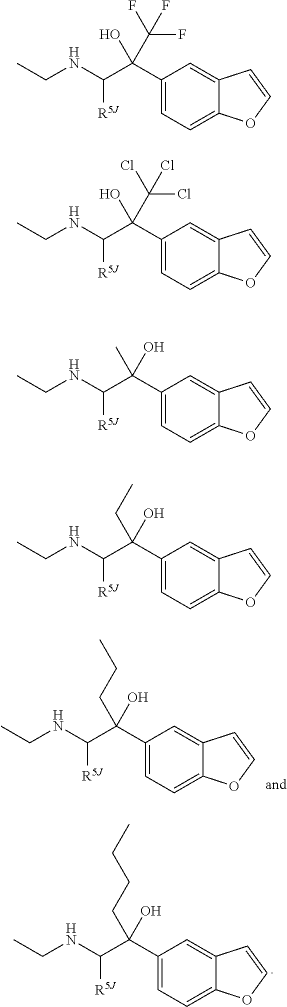

In certain embodiments the combination of the present invention includes a compound selected from:

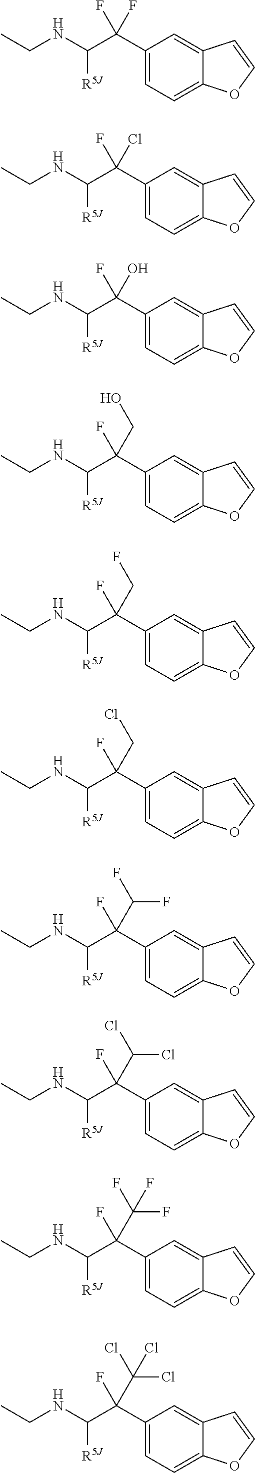

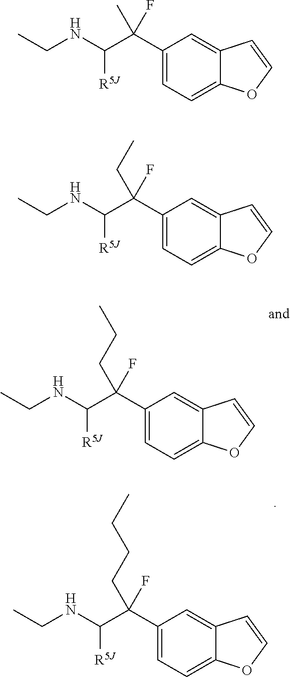

In certain embodiments the combination of the present invention includes a compound selected from:

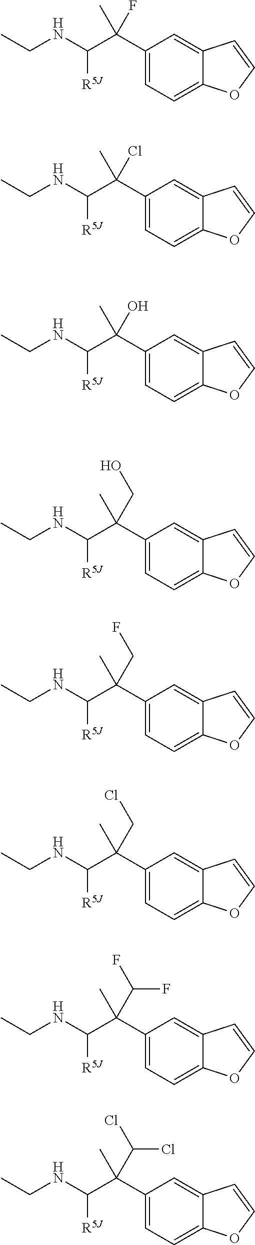

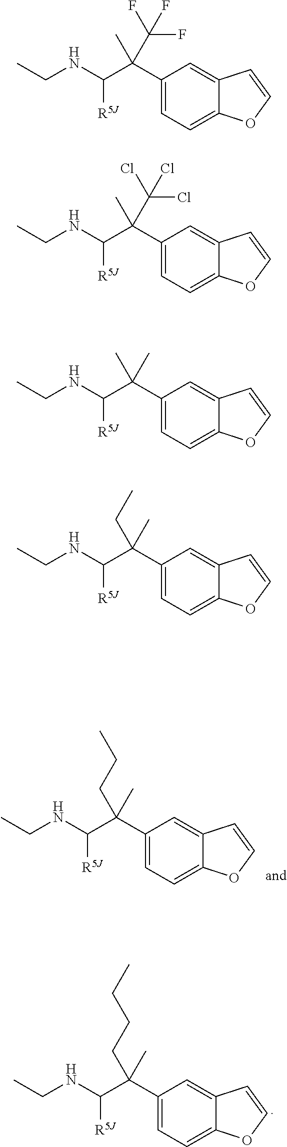

In certain embodiments the combination of the present invention includes a compound selected from:

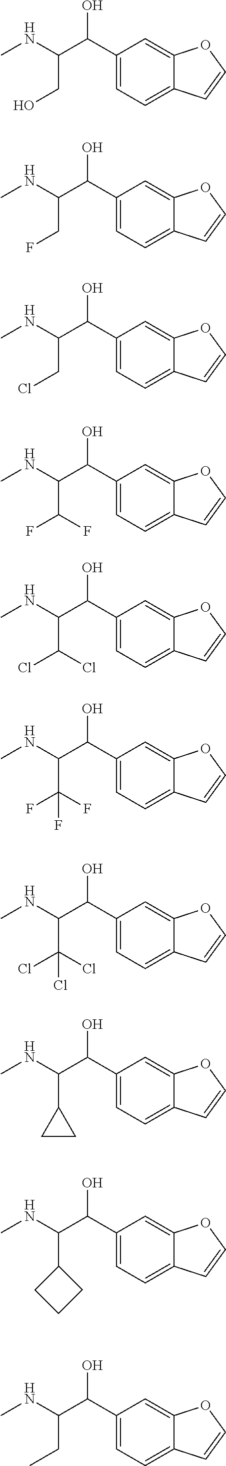

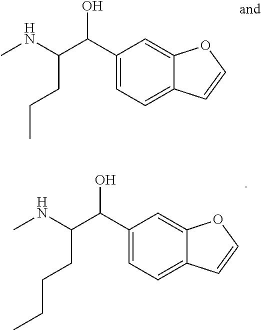 [SMILES: CCCC(NC)C(O)c1ccc2ccoc2c1 ; CCCCC(NC)C(O)c1ccc2ccoc2c1]

In certain embodiments the combination of the present invention includes a compound selected from:

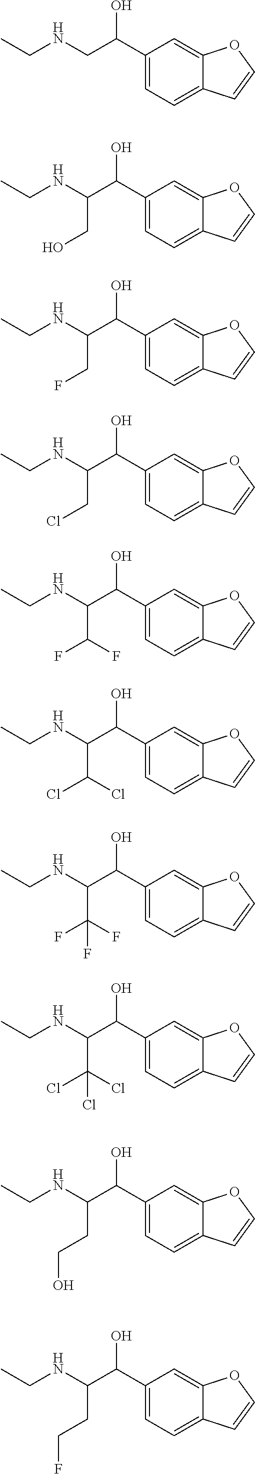

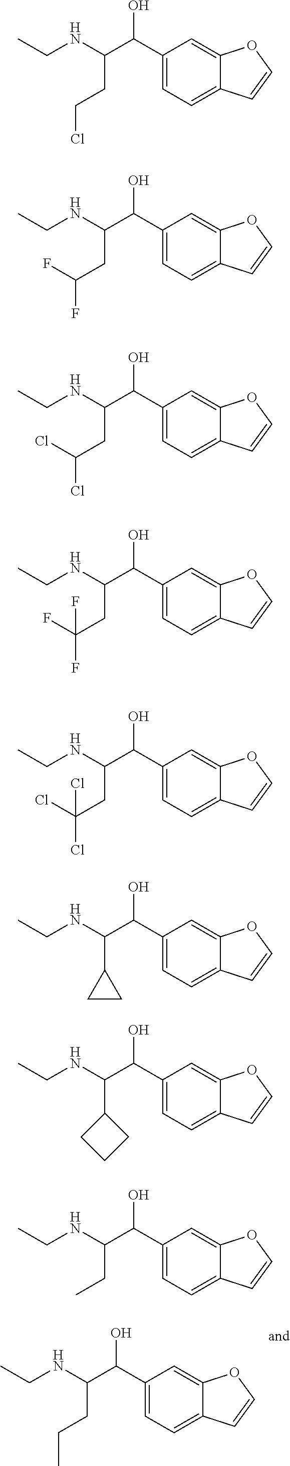

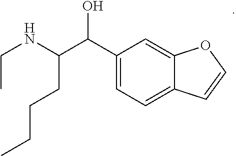 [SMILES: CCCCC(NCC)C(O)c1ccc2ccoc2c1]

In certain embodiments the combination of the present invention includes a compound selected from:

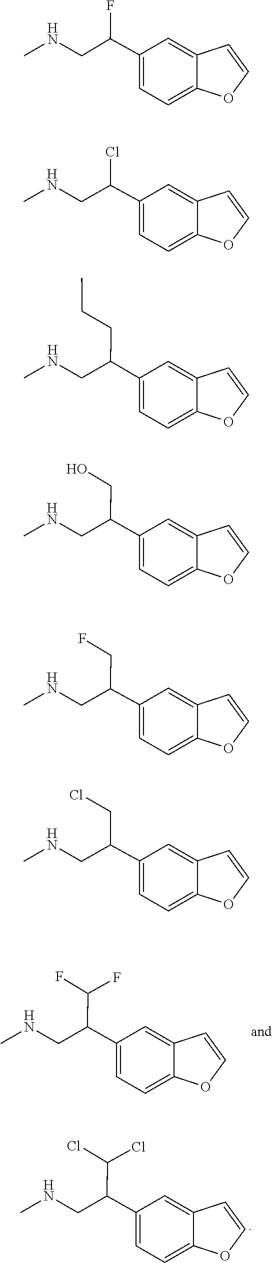

In certain embodiments the combination of the present invention includes a compound selected from:

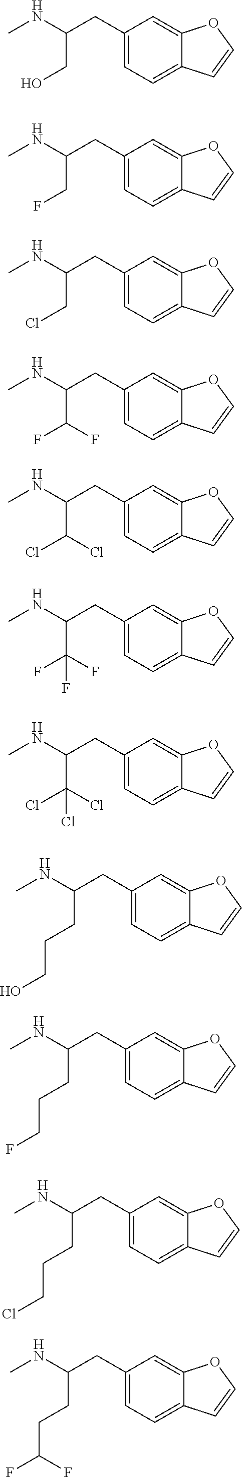

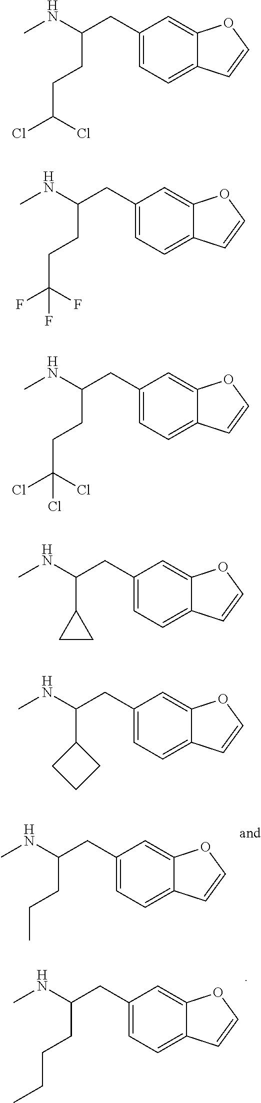

In certain embodiments the combination of the present invention includes a compound selected from:

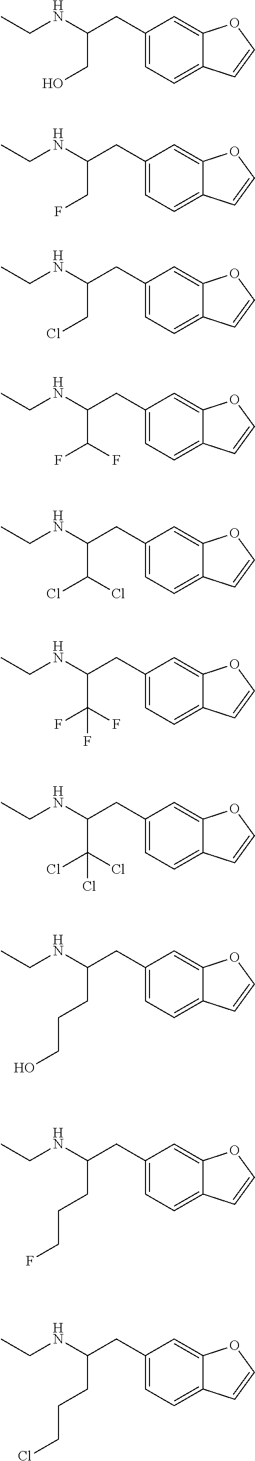

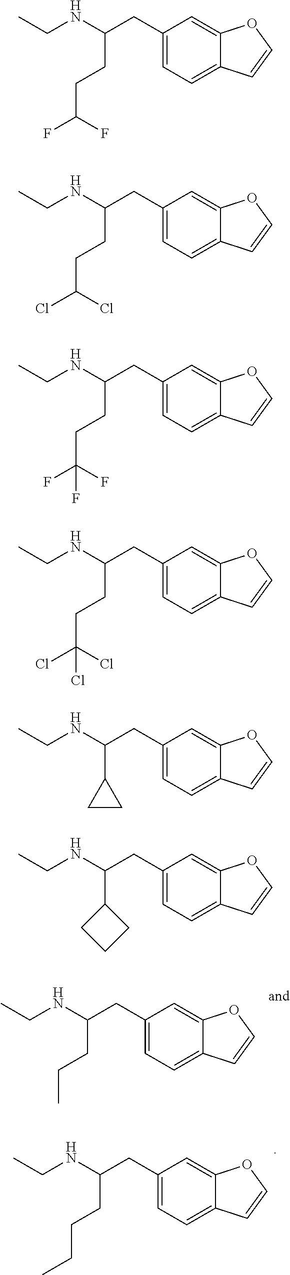

In certain embodiments the combination of the present invention includes a compound selected from:

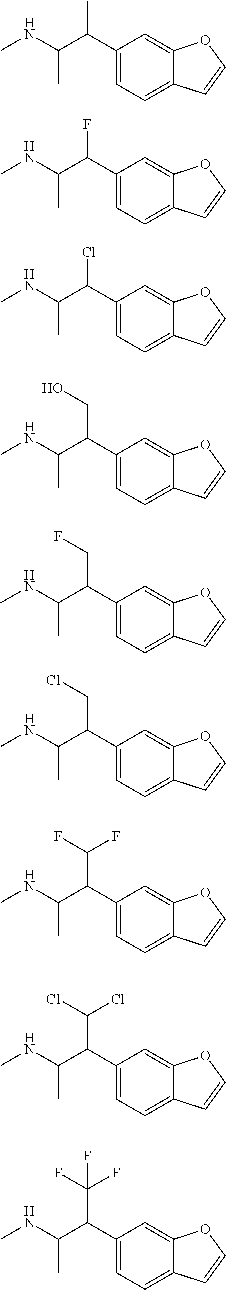

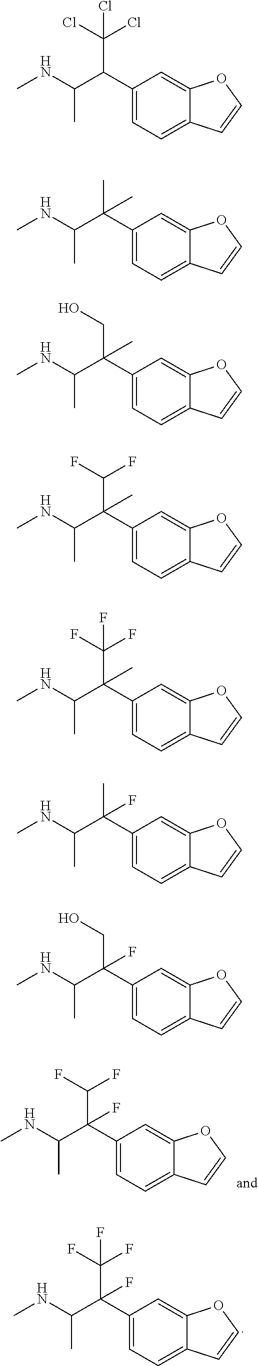

In certain embodiments the combination of the present invention includes a compound selected from:

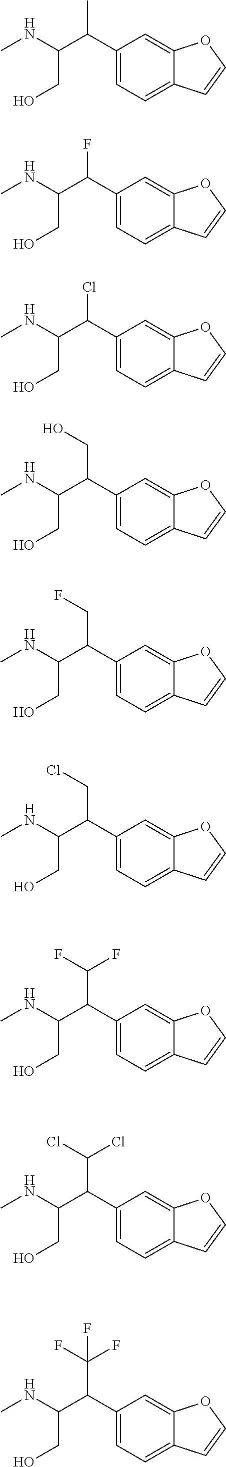

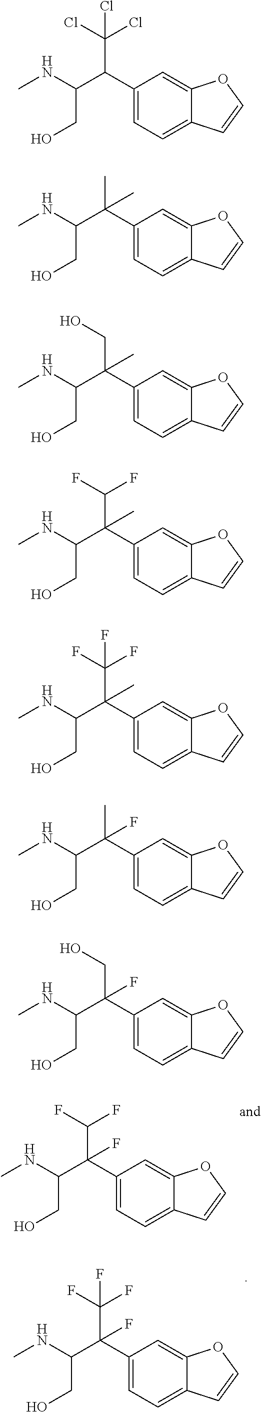

In certain embodiments the combination of the present invention includes a compound selected from:

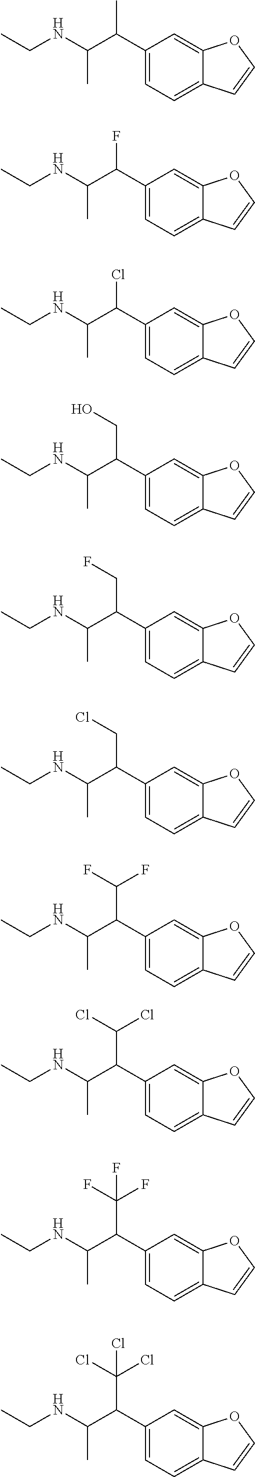

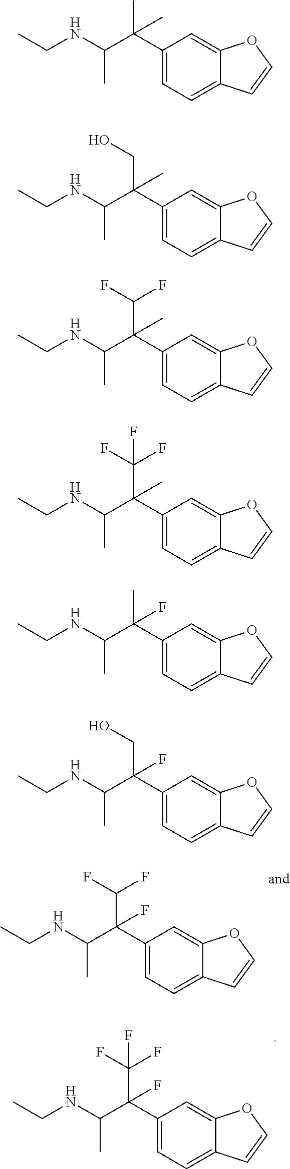

In certain embodiments the combination of the present invention includes a compound selected from:

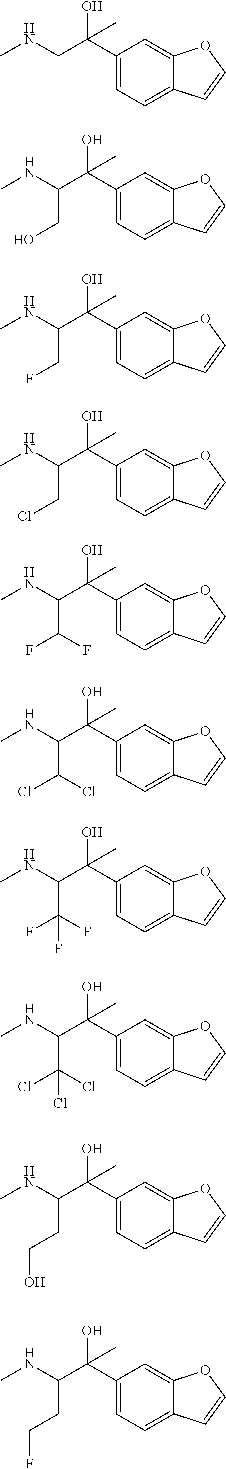

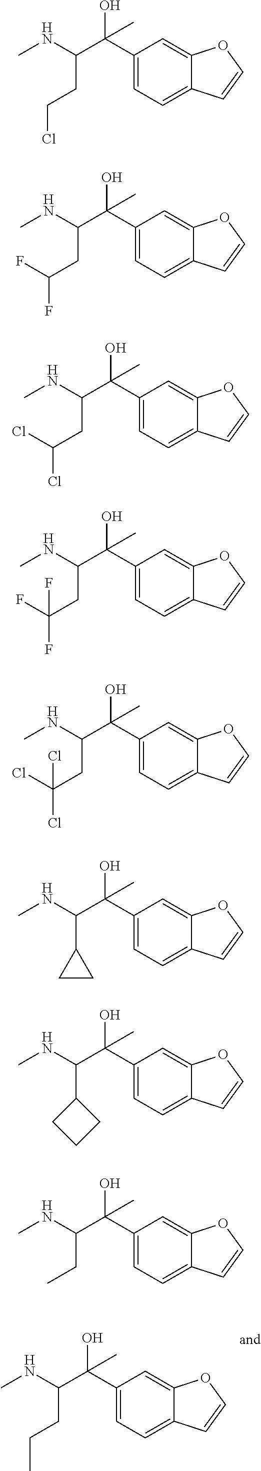

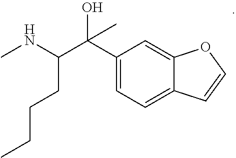 [SMILES: CCCCC(NC)C(C)(O)c1ccc2ccoc2c1]

In certain embodiments the combination of the present invention includes a compound selected from:

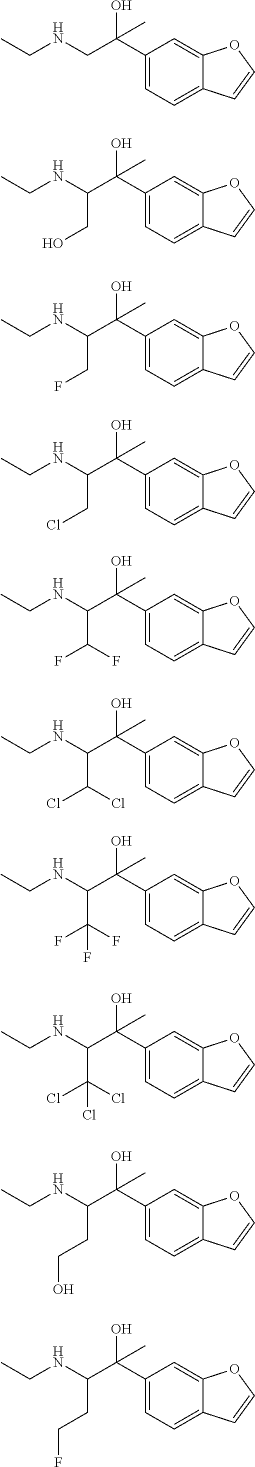

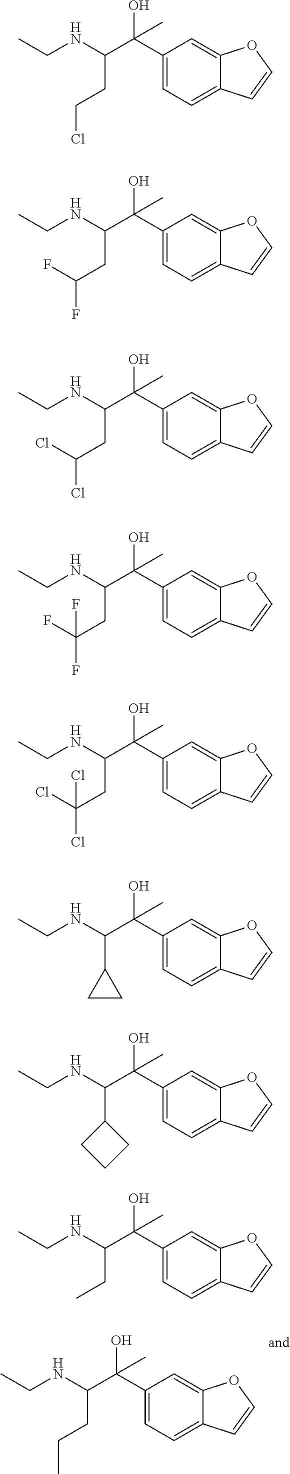

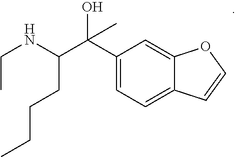 [SMILES: CCCCC(NCC)C(C)(O)c1ccc2ccoc2c1]

In certain embodiments the combination of the present invention includes a compound selected from:

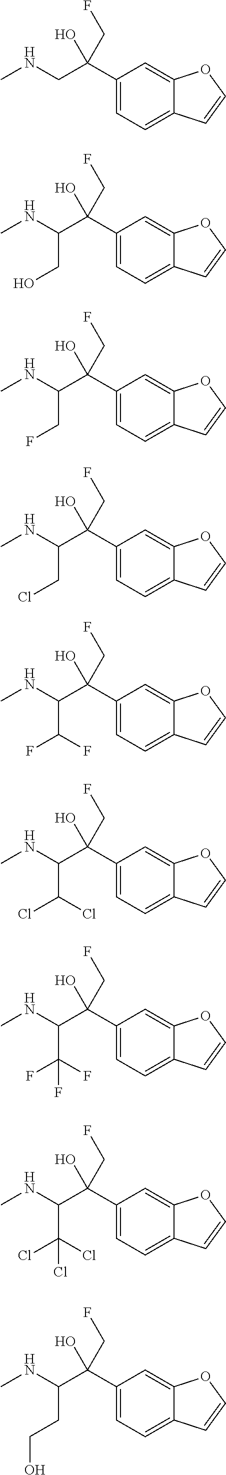

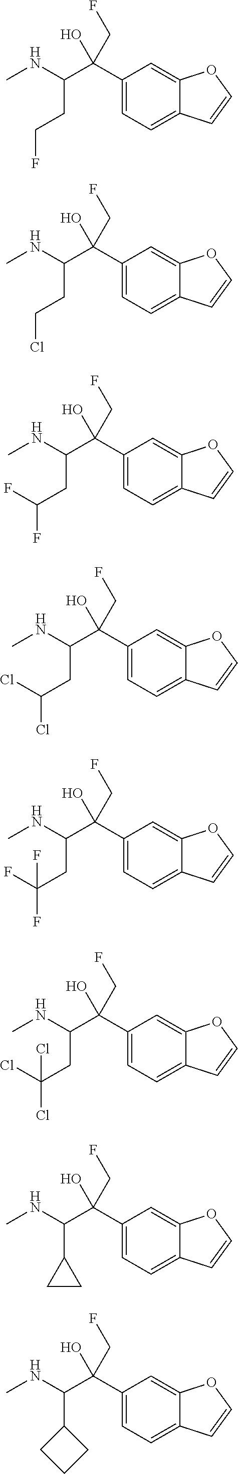

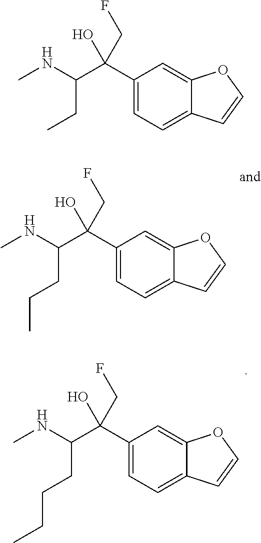 [SMILES: CCC(NC)C(O)(CF)c1ccc2ccoc2c1 ; CCCC(NC)C(O)(CF)c1ccc2ccoc2c1 ; CCCCC(NC)C(O)(CF)c1ccc2ccoc2c1]

In certain embodiments the combination of the present invention includes a compound selected from:

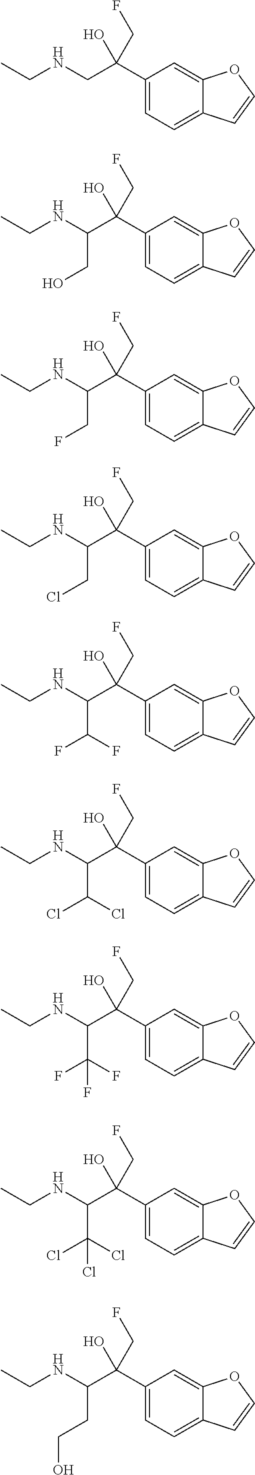

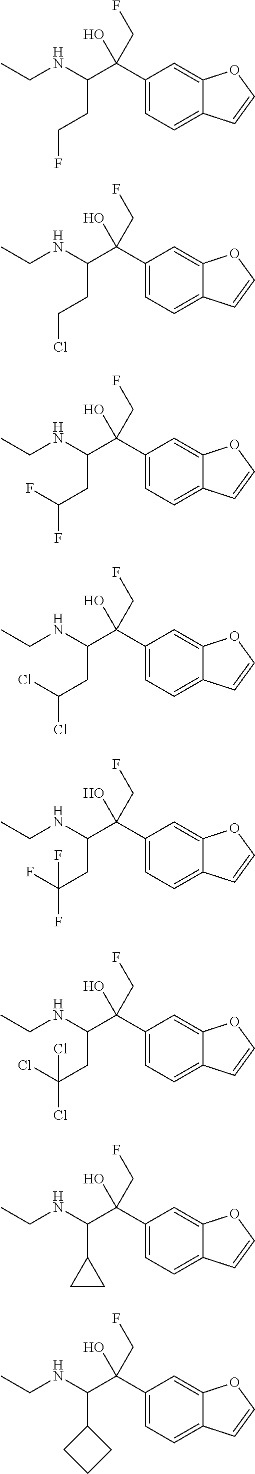

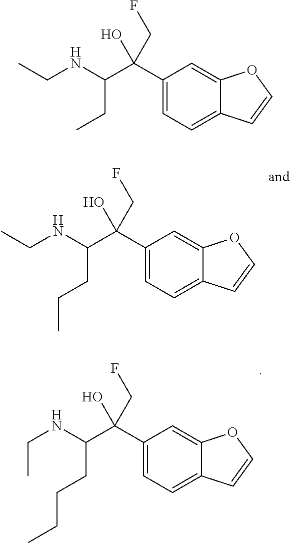 [SMILES: CCNC(CC)C(O)(CF)c1ccc2ccoc2c1 ; CCCC(NCC)C(O)(CF)c1ccc2ccoc2c1 ; CCCCC(NCC)C(O)(CF)c1ccc2ccoc2c1]

In certain embodiments the combination of the present invention includes a compound selected from:

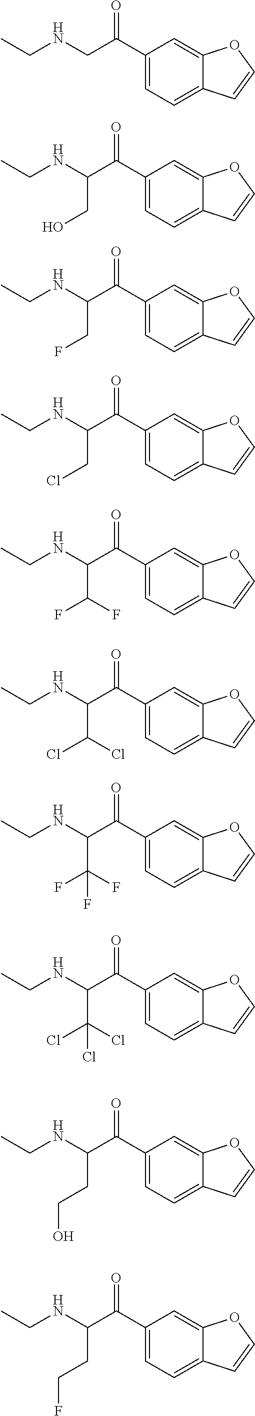

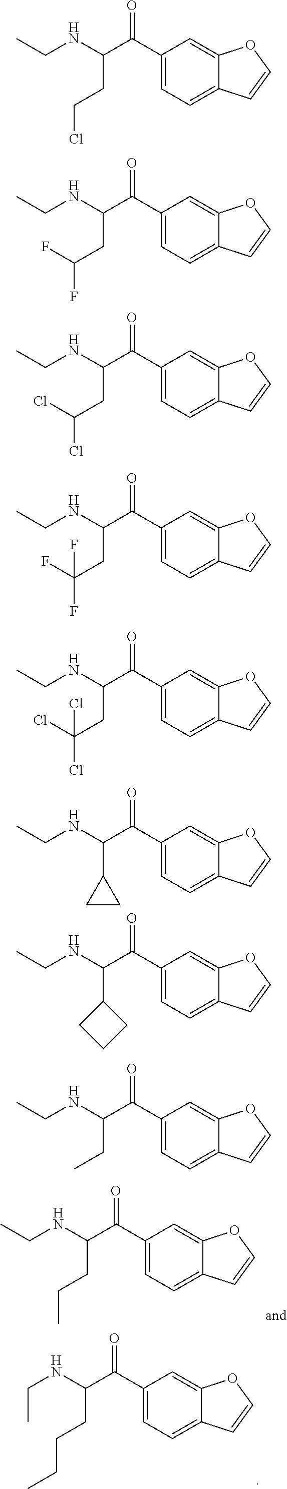

In certain embodiments the combination of the present invention includes a compound selected from:

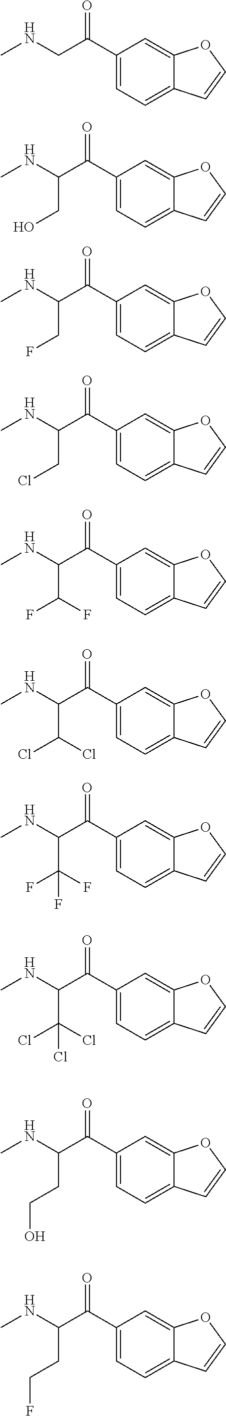

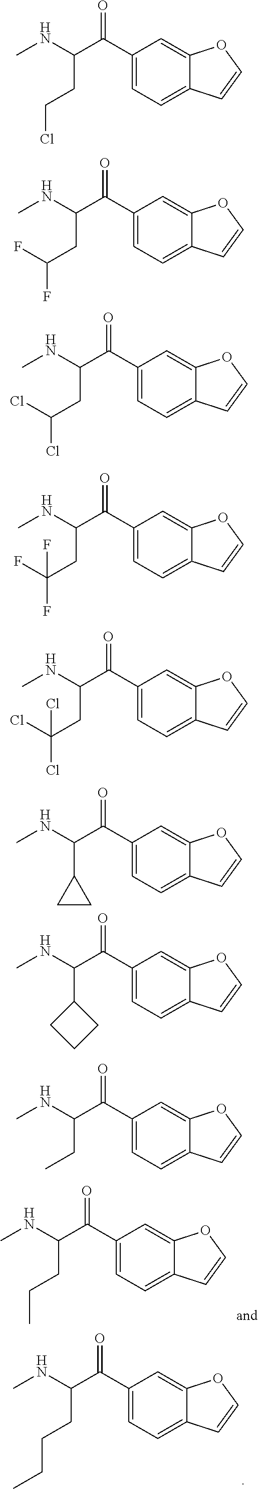

In certain embodiments the combination of the present invention includes a compound selected from:

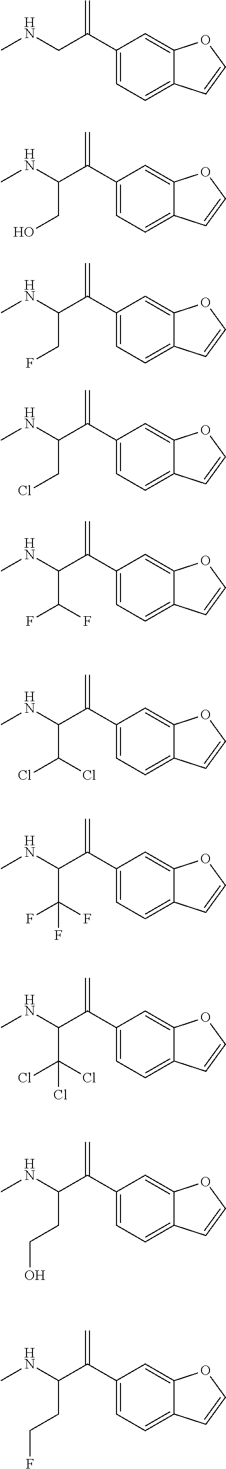

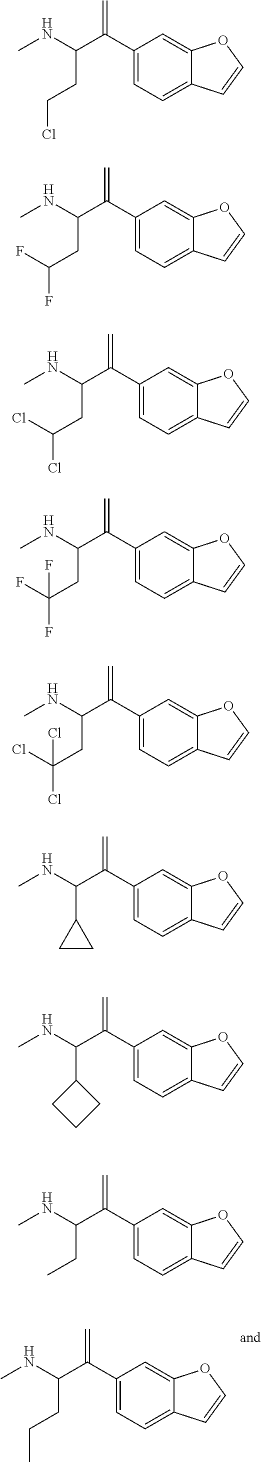

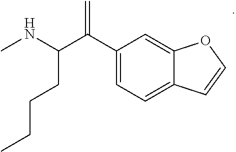 [SMILES: C=C(c1ccc2ccoc2c1)C(CCCC)NC]

In certain embodiments the combination of the present invention includes a compound selected from:

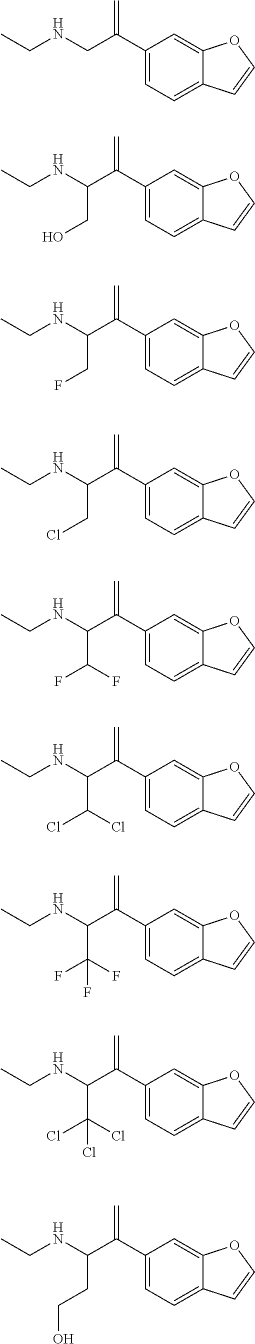

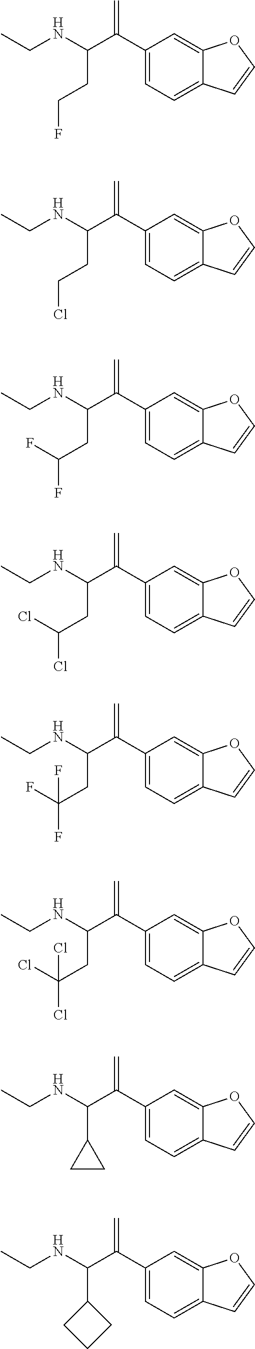

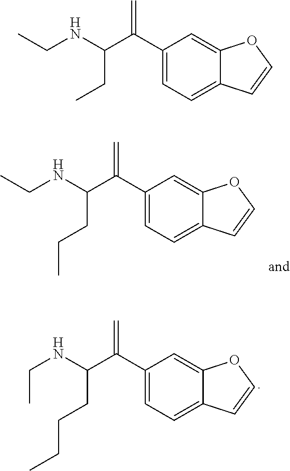 [SMILES: C=C(c1ccc2ccoc2c1)C(CC)NCC ; C=C(c1ccc2ccoc2c1)C(CCC)NCC ; C=C(c1ccc2ccoc2c1)C(CCCC)NCC]

In certain embodiments the combination of the present invention includes a compound selected from:

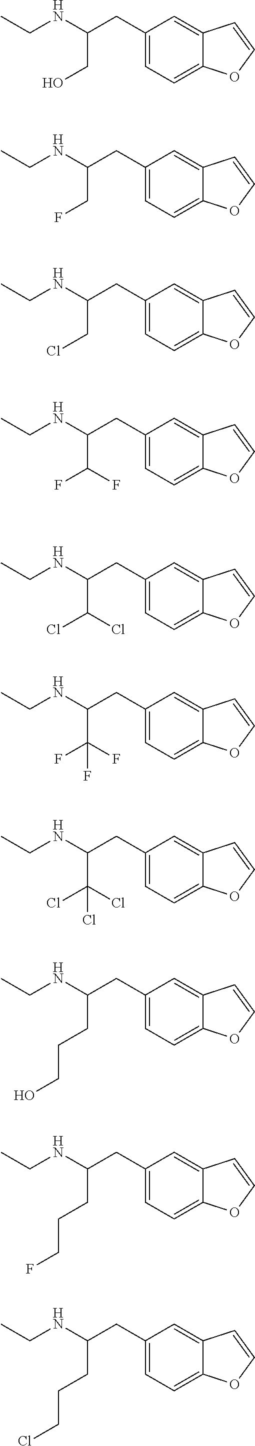

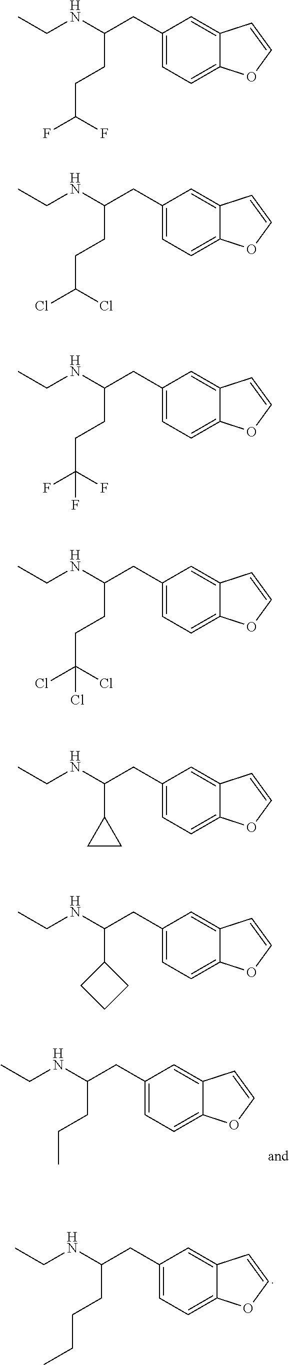

In certain embodiments the combination of the present invention includes a compound selected from:

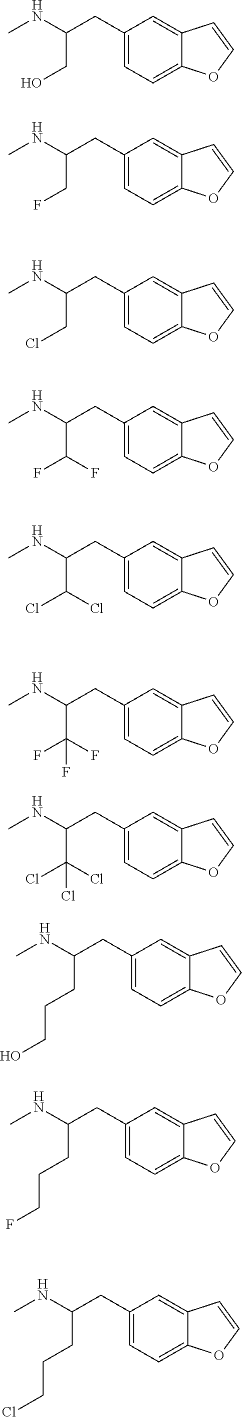

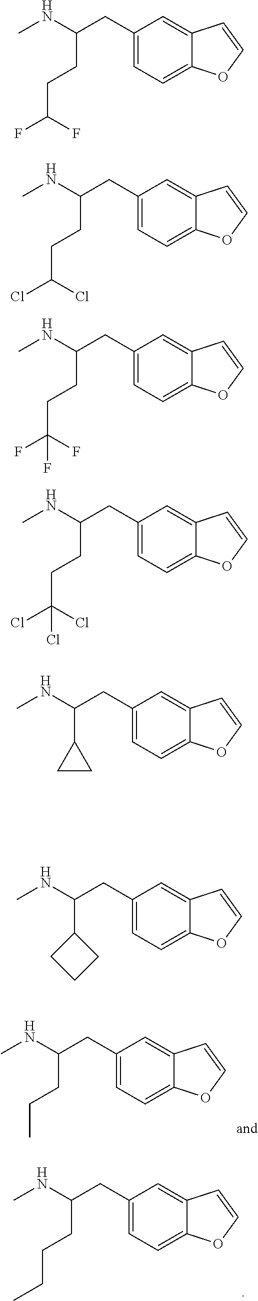

In certain embodiments the combination of the present invention includes a compound selected from:

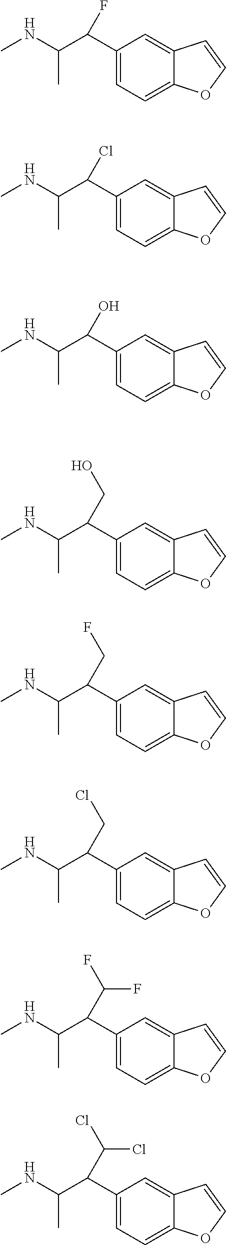

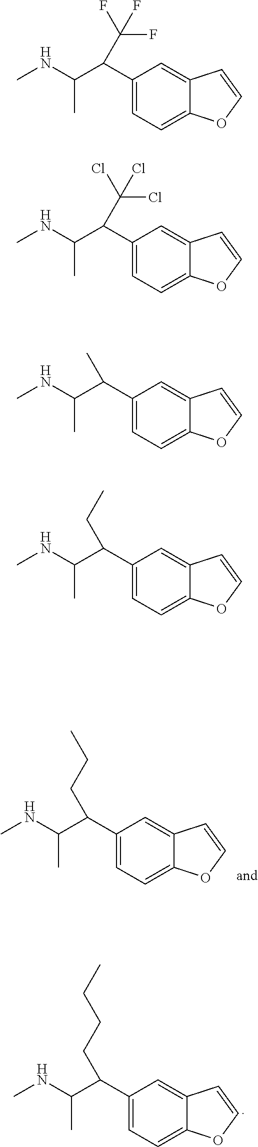

In certain embodiments the combination of the present invention includes a compound selected from:

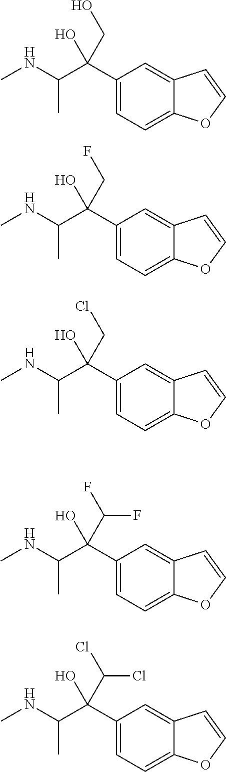 [SMILES: CNC(C)C(O)(CO)c1ccc2occc2c1 ; CNC(C)C(O)(CF)c1ccc2occc2c1 ; CNC(C)C(O)(CCl)c1ccc2occc2c1 ; CNC(C)C(O)(c1ccc2occc2c1)C(F)F ; CNC(C)C(O)(c1ccc2occc2c1)C(Cl)Cl]

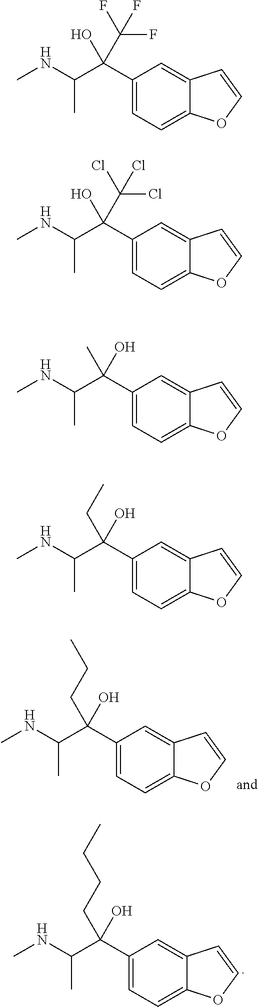

In certain embodiments the combination of the present invention includes a compound selected from:

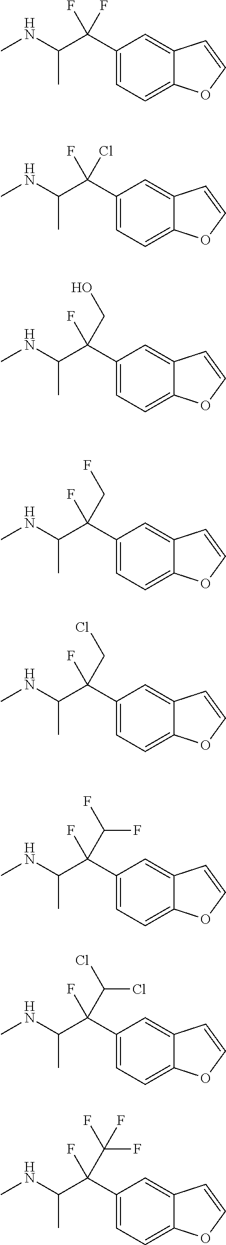

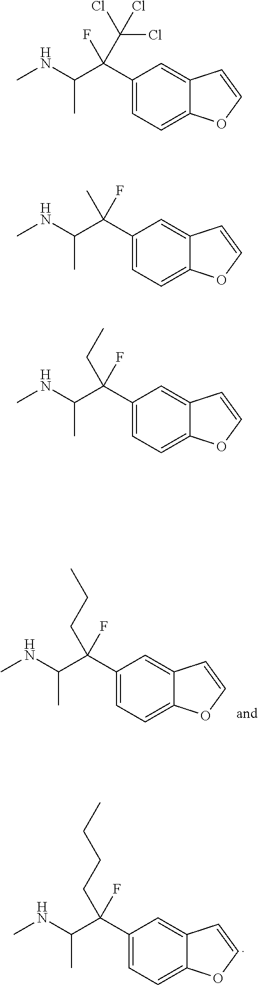 [SMILES: CNC(C)C(F)(c1ccc2occc2c1)C(Cl)(Cl)Cl ; CNC(C)C(C)(F)c1ccc2occc2c1 ; CCC(F)(c1ccc2occc2c1)C(C)NC ; CCCC(F)(c1ccc2occc2c1)C(C)NC ; CCCCC(F)(c1ccc2occc2c1)C(C)NC]

In certain embodiments the combination of the present invention includes a compound selected from:

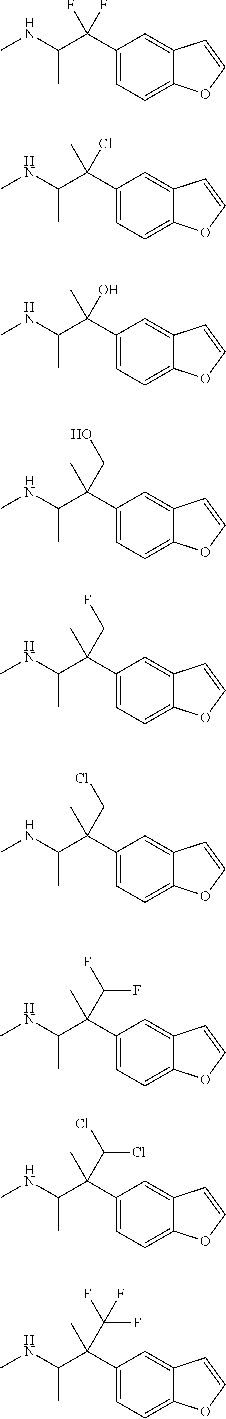

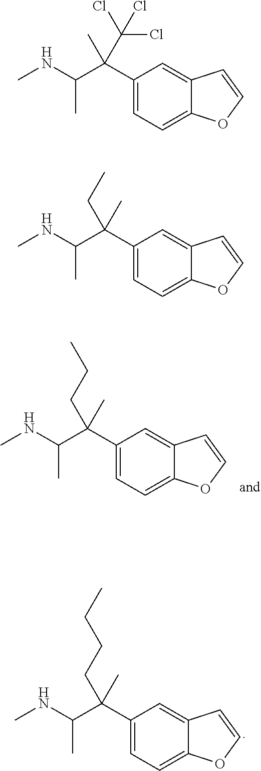 [SMILES: CNC(C)C(C)(c1ccc2occc2c1)C(Cl)(Cl)Cl ; CCC(C)(c1ccc2occc2c1)C(C)NC ; CCCC(C)(c1ccc2occc2c1)C(C)NC ; CCCCC(C)(c1ccc2occc2c1)C(C)NC]

In certain embodiments the combination of the present invention includes a compound selected from:

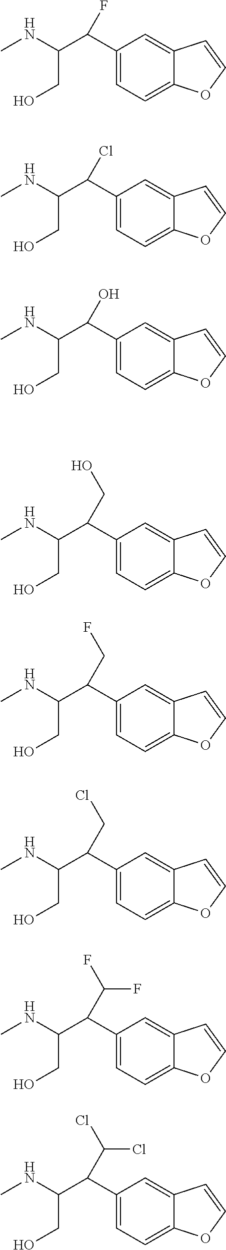

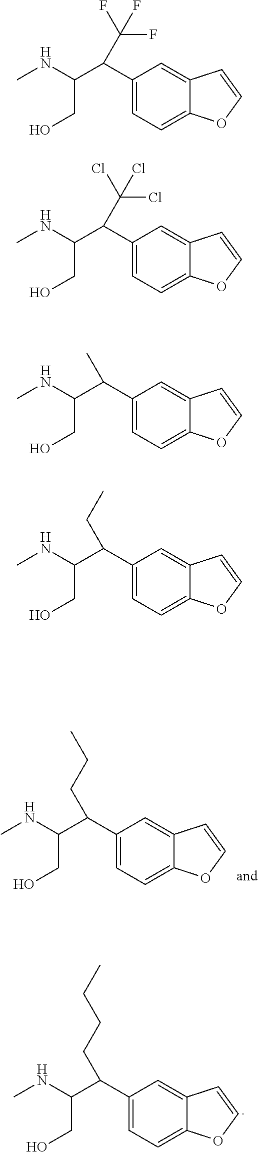

In certain embodiments the combination of the present invention includes a compound selected from:

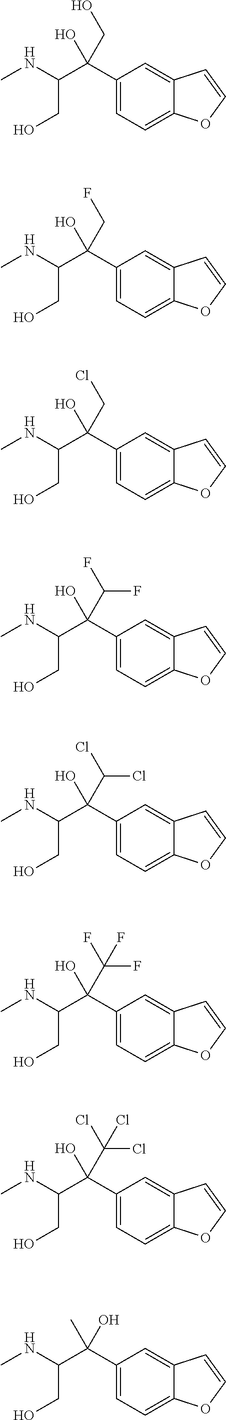

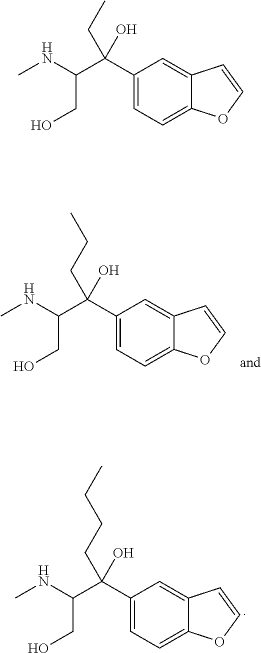 [SMILES: CCC(O)(c1ccc2occc2c1)C(CO)NC ; CCCC(O)(c1ccc2occc2c1)C(CO)NC ; CCCCC(O)(c1ccc2occc2c1)C(CO)NC]

In certain embodiments the combination of the present invention includes a compound selected from:

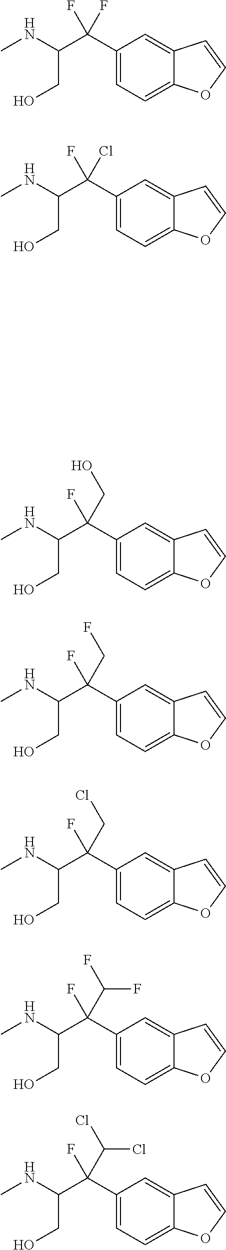

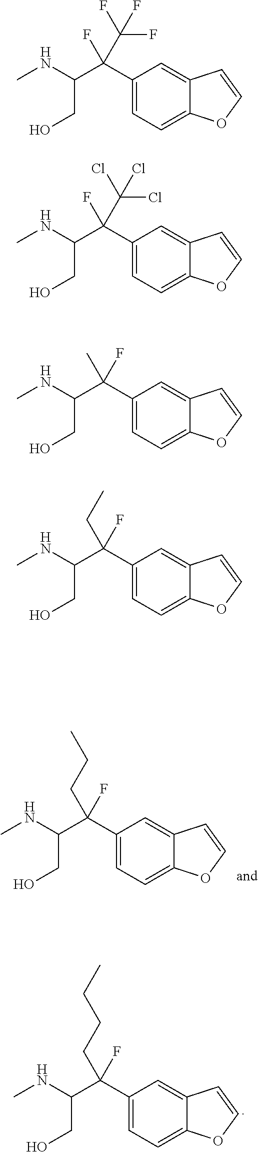

In certain embodiments the combination of the present invention includes a compound selected from:

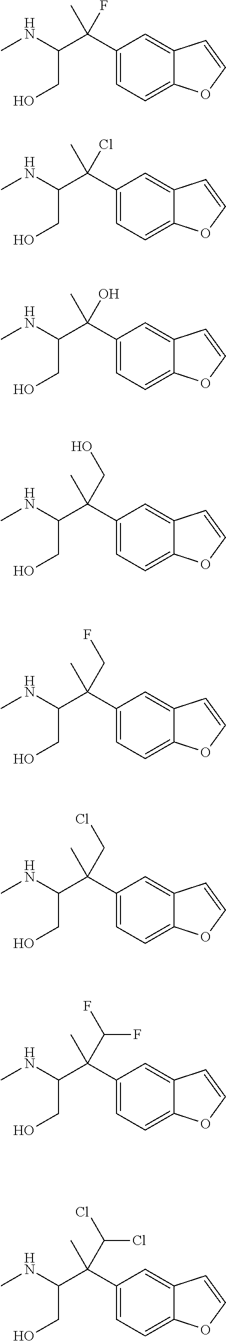

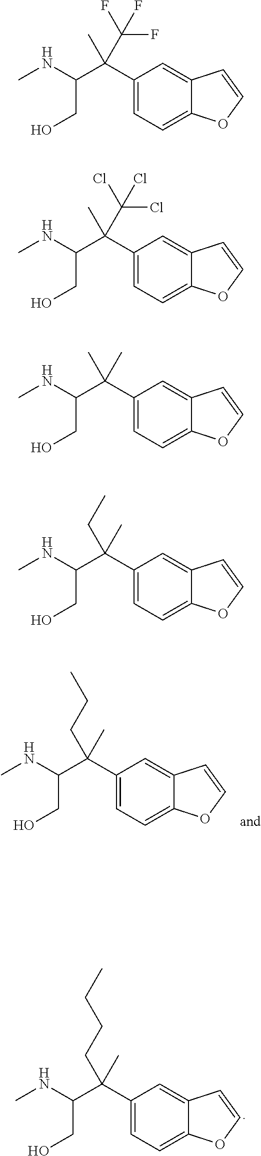 [SMILES: CNC(CO)C(C)(c1ccc2occc2c1)C(F)(F)F ; CNC(CO)C(C)(c1ccc2occc2c1)C(Cl)(Cl)Cl ; CNC(CO)C(C)(C)c1ccc2occc2c1 ; CCC(C)(c1ccc2occc2c1)C(CO)NC ; CCCC(C)(c1ccc2occc2c1)C(CO)NC ; CCCCC(C)(c1ccc2occc2c1)C(CO)NC]

In certain embodiments the combination of the present invention includes a compound selected from:

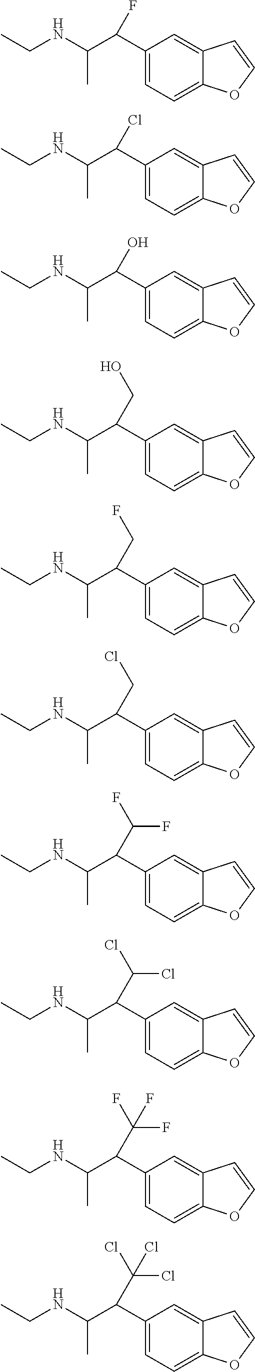

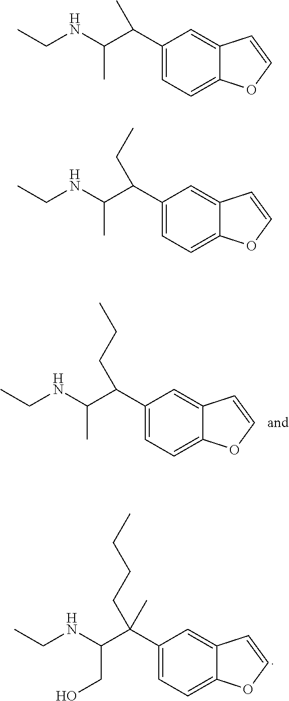 [SMILES: CCNC(C)C(C)c1ccc2occc2c1 ; CCNC(C)C(CC)c1ccc2occc2c1 ; CCCC(c1ccc2occc2c1)C(C)NCC ; CCCCC(C)(c1ccc2occc2c1)C(CO)NCC]

In certain embodiments the combination of the present invention includes a compound selected from:

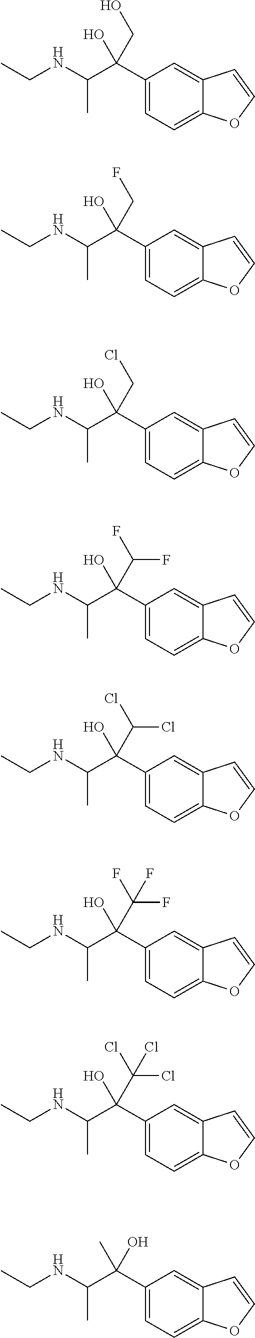

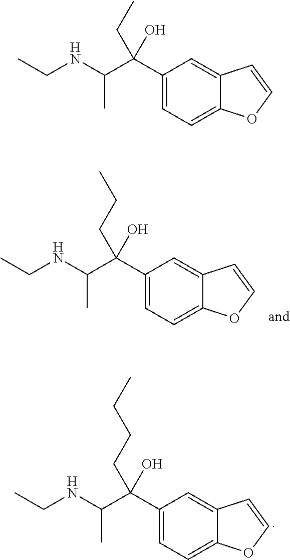 [SMILES: CCNC(C)C(O)(CC)c1ccc2occc2c1 ; CCCC(O)(c1ccc2occc2c1)C(C)NCC ; CCCCC(O)(c1ccc2occc2c1)C(C)NCC]

In certain embodiments the combination of the present invention includes a compound selected from:

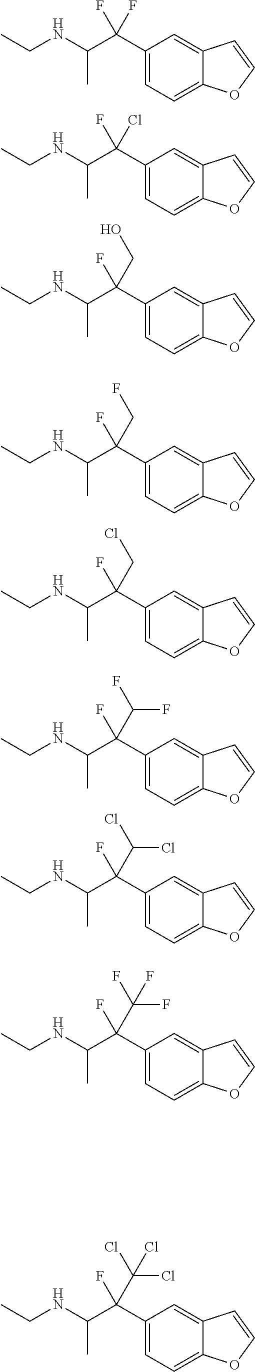

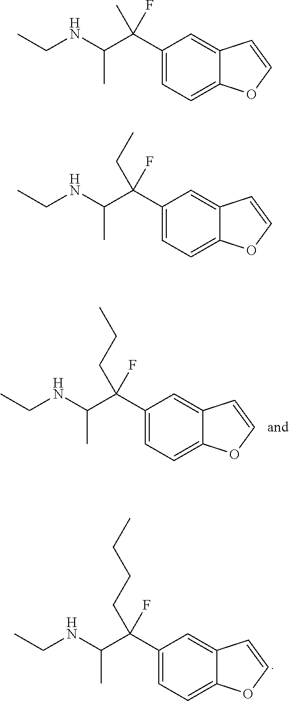 [SMILES: CCNC(C)C(C)(F)c1ccc2occc2c1 ; CCNC(C)C(F)(CC)c1ccc2occc2c1 ; CCCC(F)(c1ccc2occc2c1)C(C)NCC ; CCCCC(F)(c1ccc2occc2c1)C(C)NCC]

In certain embodiments the combination of the present invention includes a compound selected from:

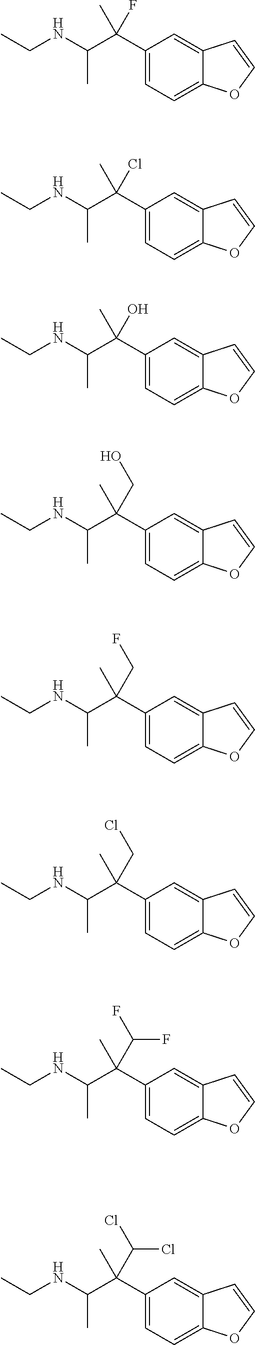

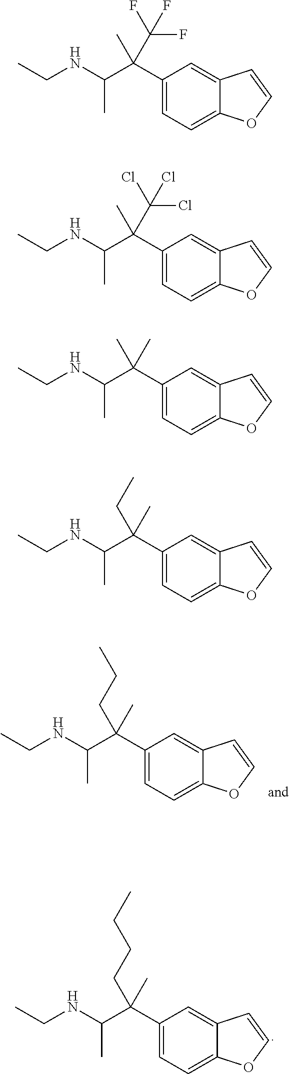 [SMILES: CCNC(C)C(C)(c1ccc2occc2c1)C(F)(F)F ; CCNC(C)C(C)(c1ccc2occc2c1)C(Cl)(Cl)Cl ; CCNC(C)C(C)(C)c1ccc2occc2c1 ; CCNC(C)C(C)(CC)c1ccc2occc2c1 ; CCCC(C)(c1ccc2occc2c1)C(C)NCC ; CCCCC(C)(c1ccc2occc2c1)C(C)NCC]

In certain embodiments the combination of the present invention includes a compound selected from:

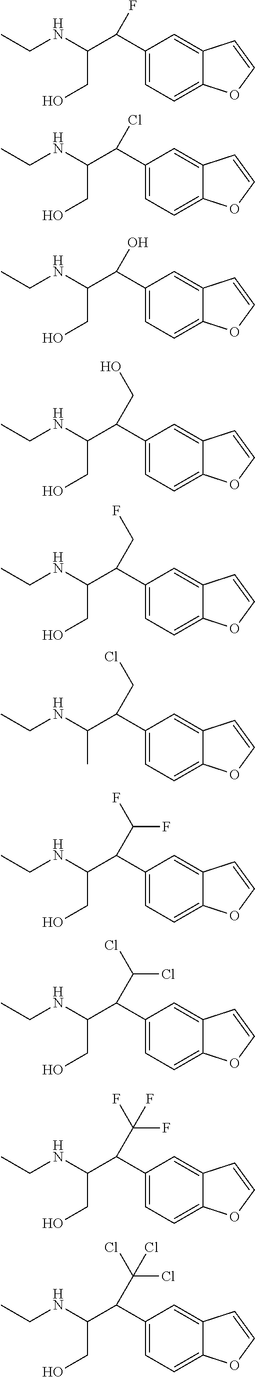

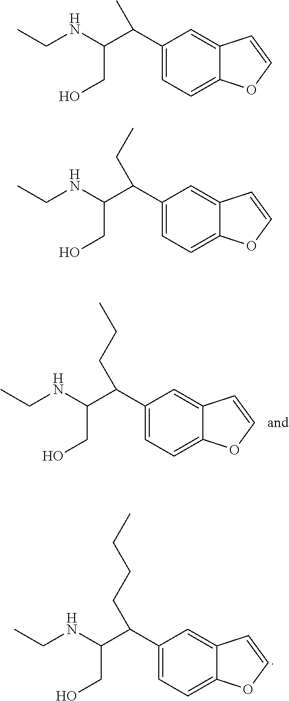 [SMILES: CCNC(CO)C(C)c1ccc2occc2c1 ; CCNC(CO)C(CC)c1ccc2occc2c1 ; CCCC(c1ccc2occc2c1)C(CO)NCC ; CCCCC(c1ccc2occc2c1)C(CO)NCC]

In certain embodiments the combination of the present invention includes a compound selected from:

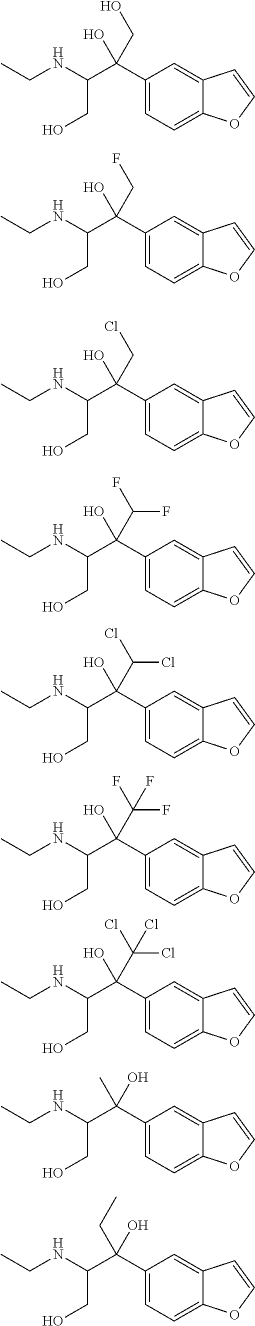

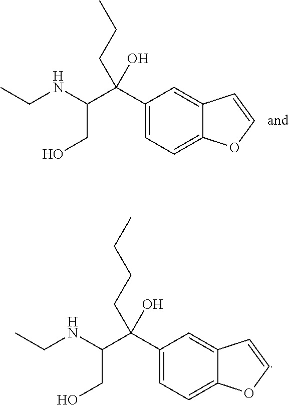 [SMILES: CCCC(O)(c1ccc2occc2c1)C(CO)NCC ; CCCCC(O)(c1ccc2occc2c1)C(CO)NCC]

In certain embodiments the combination of the present invention includes a compound selected from:

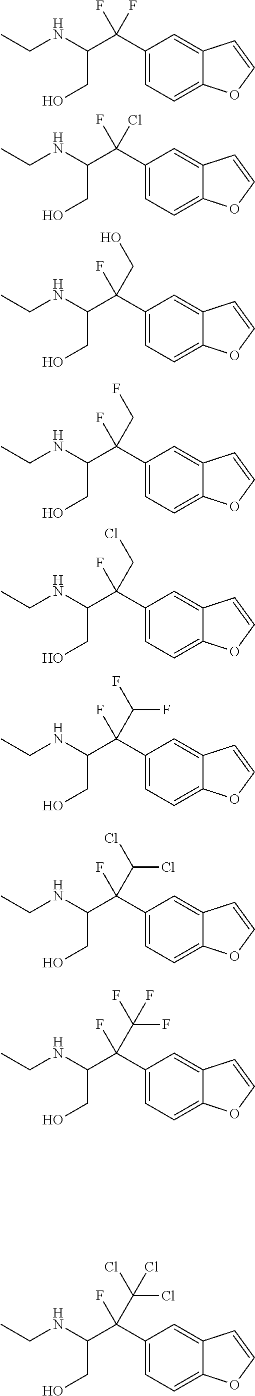

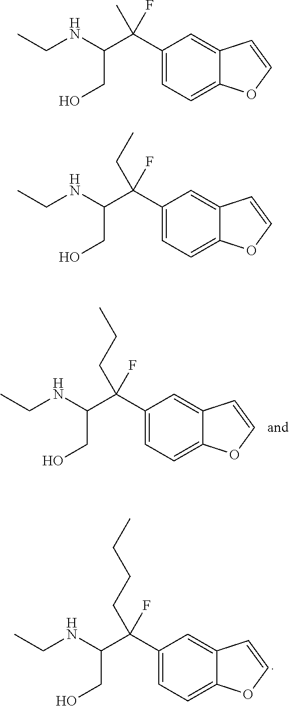 [SMILES: CCNC(CO)C(C)(F)c1ccc2occc2c1 ; CCNC(CO)C(F)(CC)c1ccc2occc2c1 ; CCCC(F)(c1ccc2occc2c1)C(CO)NCC ; CCCCC(F)(c1ccc2occc2c1)C(CO)NCC]

In certain embodiments the combination of the present invention includes a compound selected from:

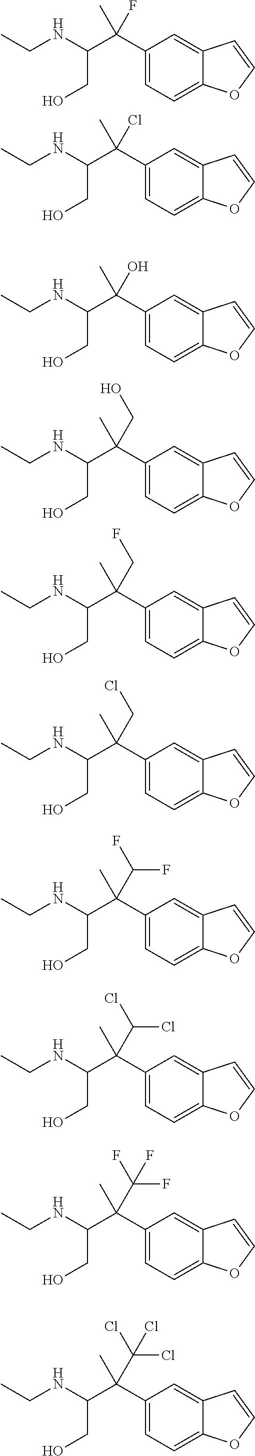

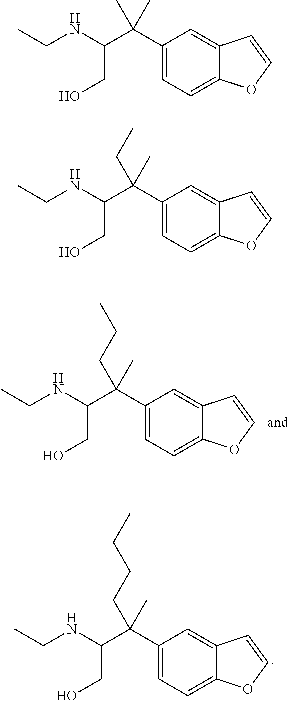 [SMILES: CCNC(CO)C(C)(C)c1ccc2occc2c1 ; CCNC(CO)C(C)(CC)c1ccc2occc2c1 ; CCCC(C)(c1ccc2occc2c1)C(CO)NCC ; CCCCC(C)(c1ccc2occc2c1)C(CO)NCC]

In certain embodiments the combination of the present invention includes a compound selected from:

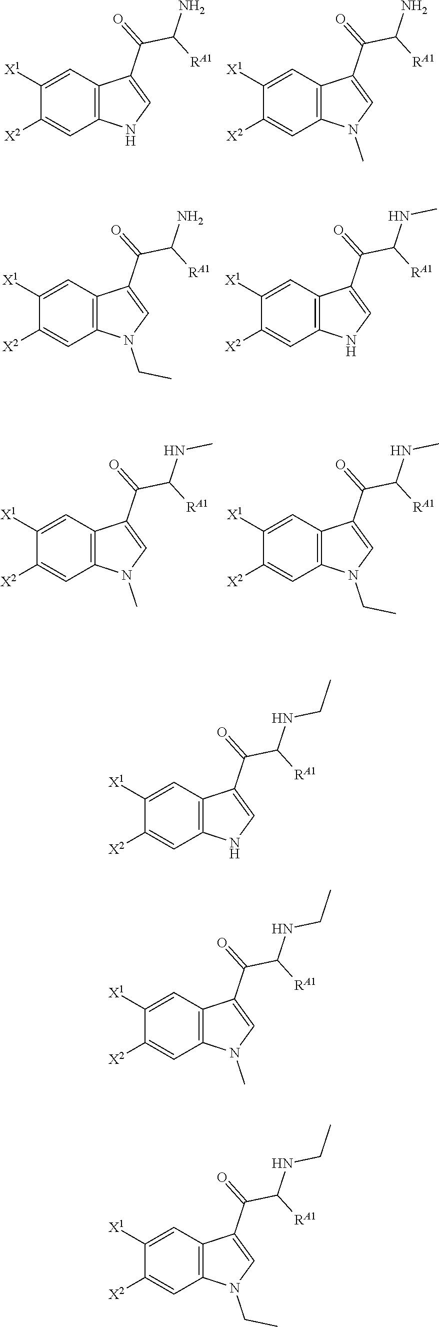

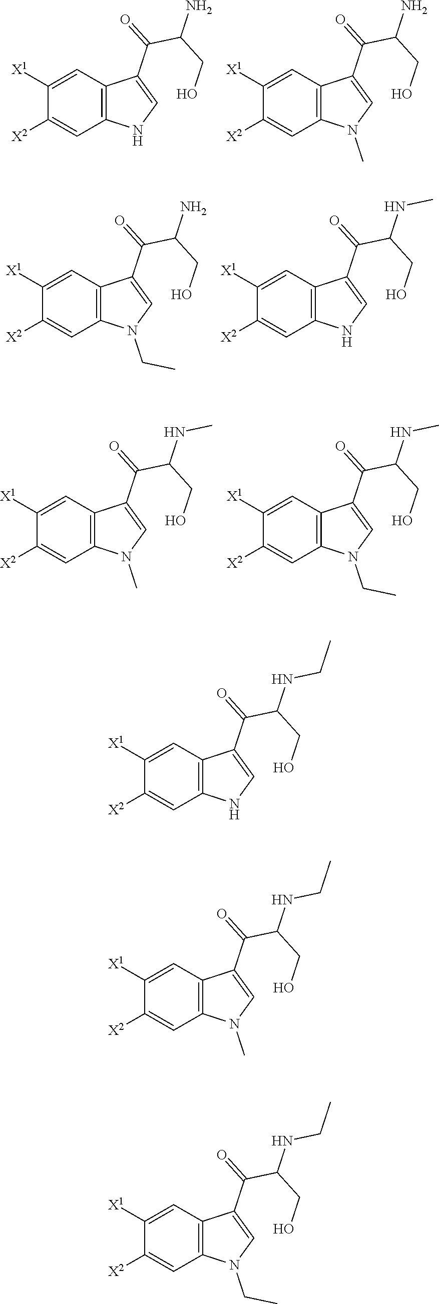

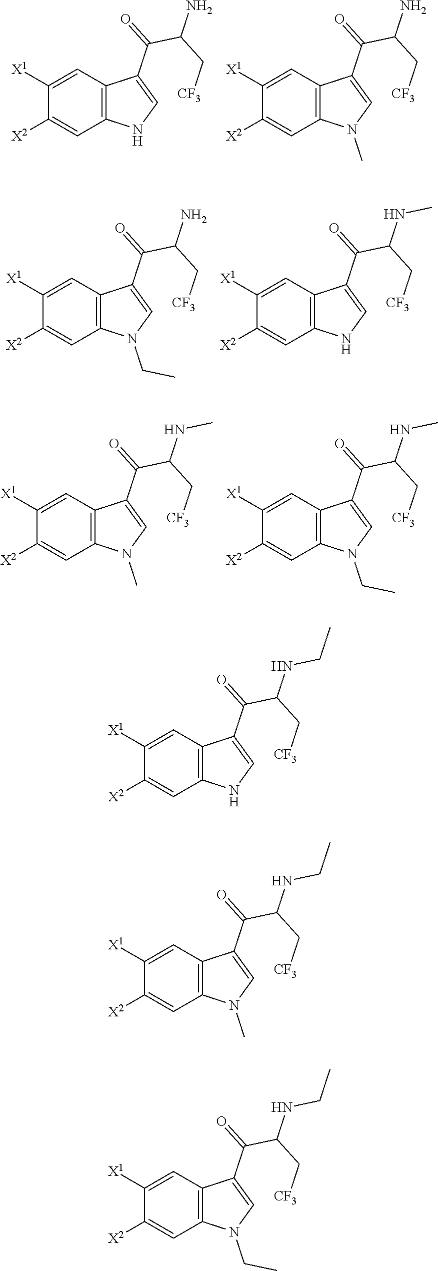

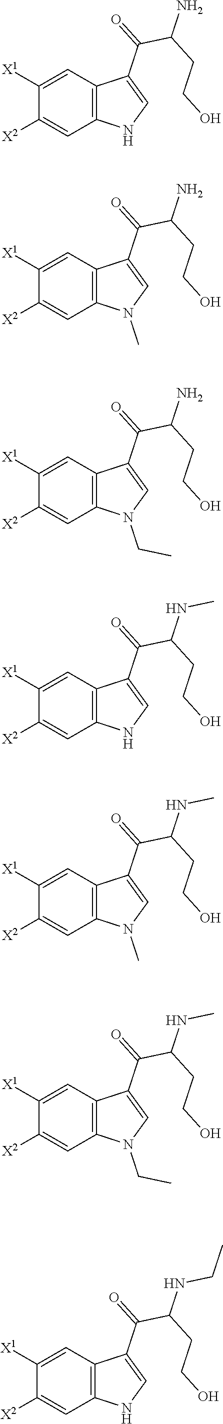

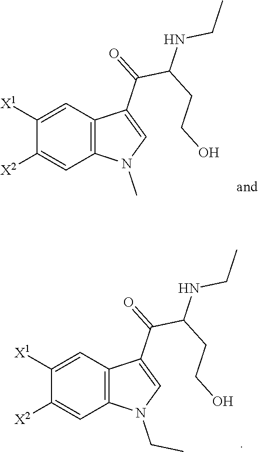

In certain embodiments the combination of the present invention includes a compound selected from:

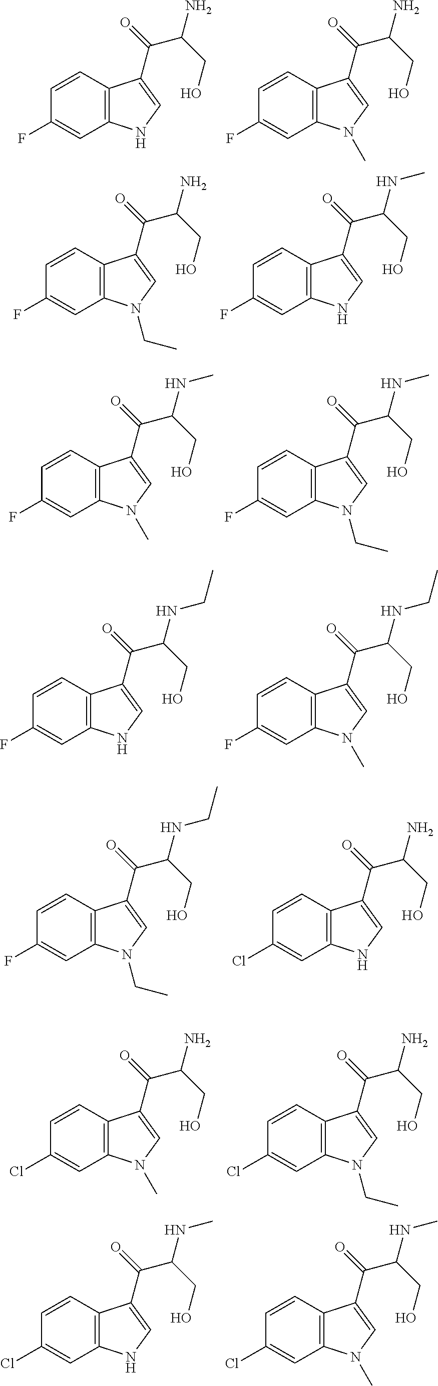

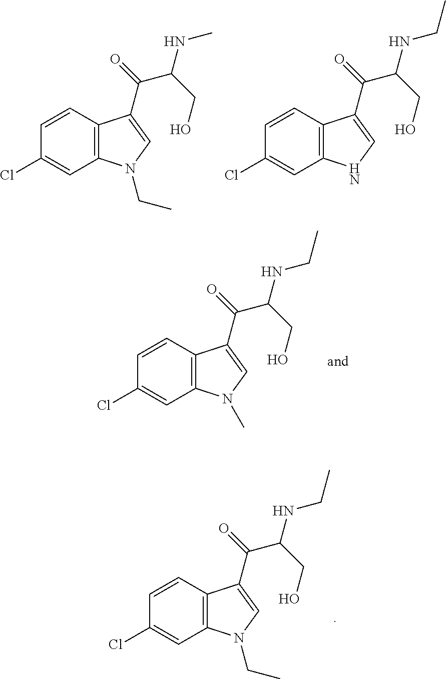 [SMILES: CCn1cc(C(=O)C(CO)NC)c2ccc(Cl)cc21 ; CCNC(CO)C(=O)c1c[nH]c2cc(Cl)ccc12 ; CCNC(CO)C(=O)c1cn(C)c2cc(Cl)ccc12 ; CCNC(CO)C(=O)c1cn(CC)c2cc(Cl)ccc12]

In certain embodiments the combination of the present invention includes a compound selected from:

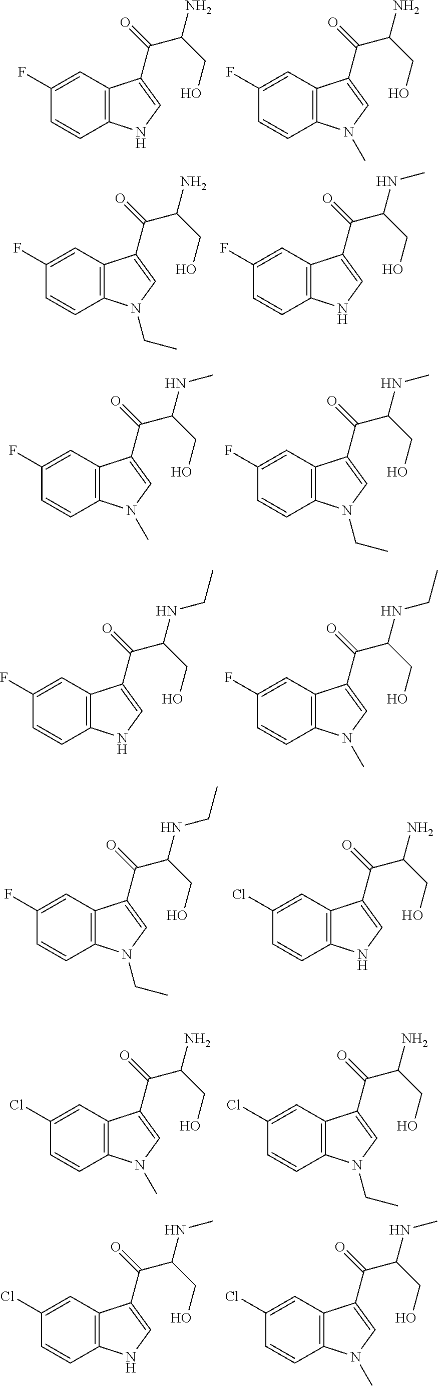

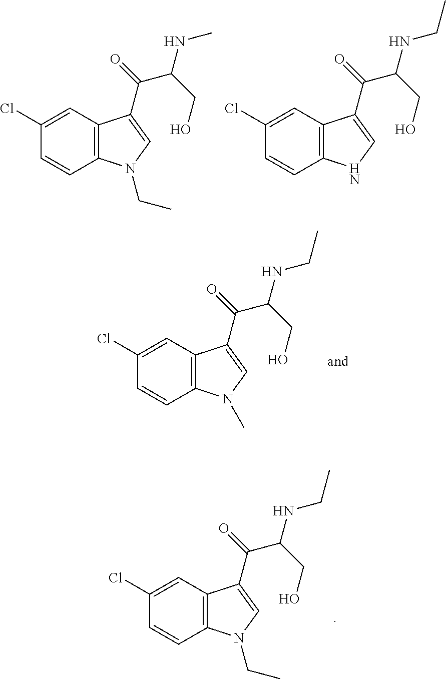

In certain embodiments the combination of the present invention includes a compound selected from:

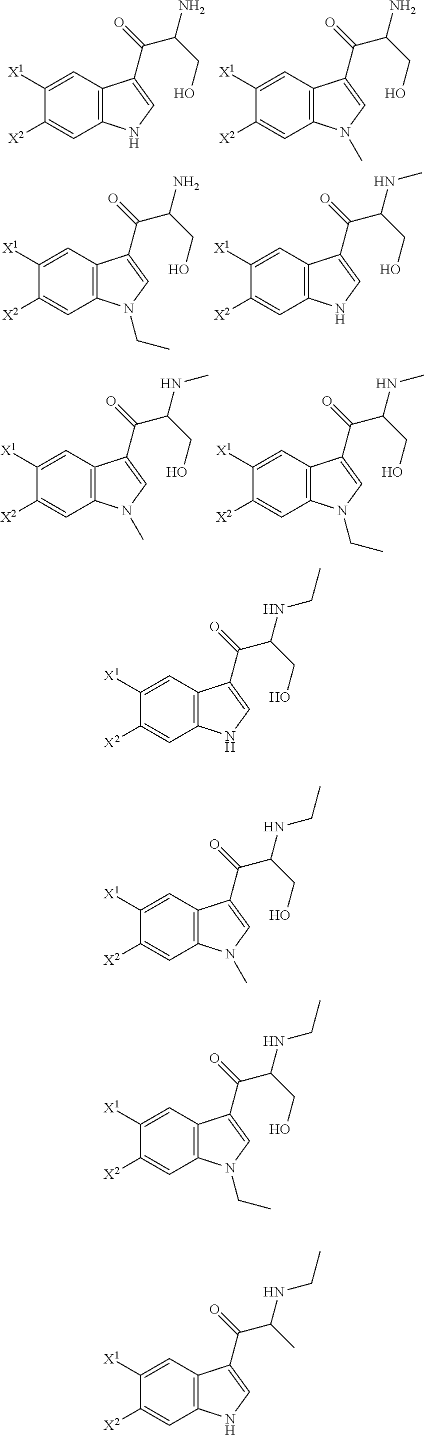

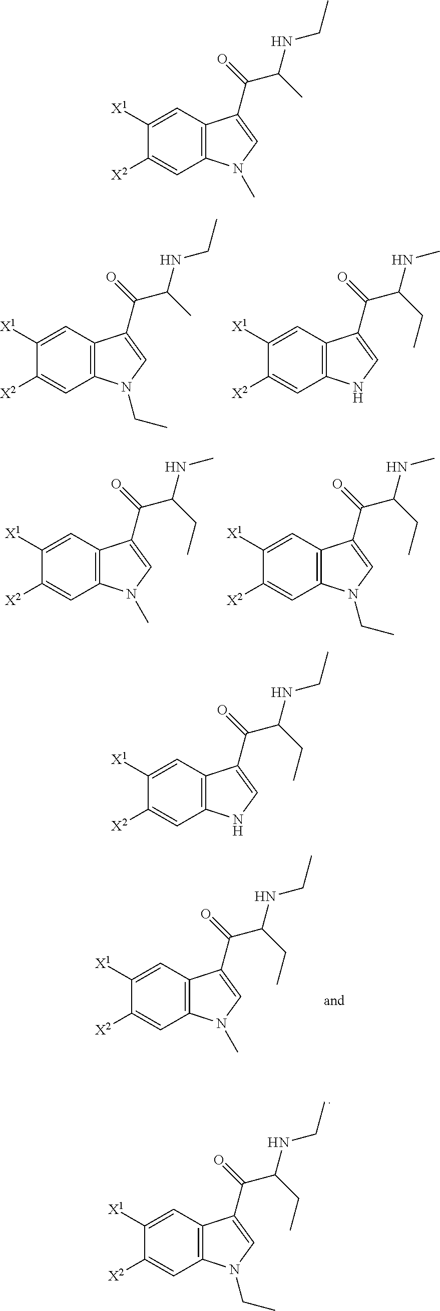

In certain embodiments the combination of the present invention includes a compound selected from:

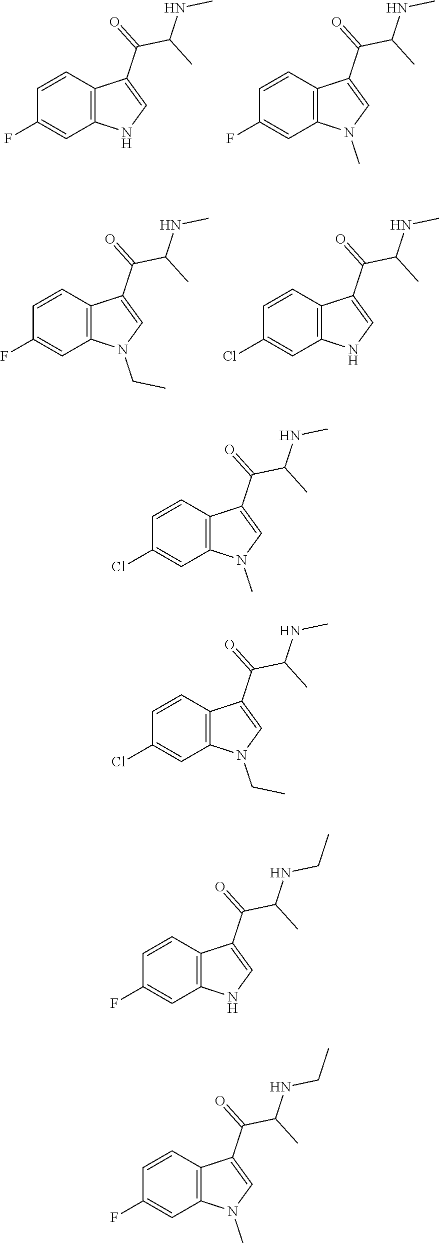

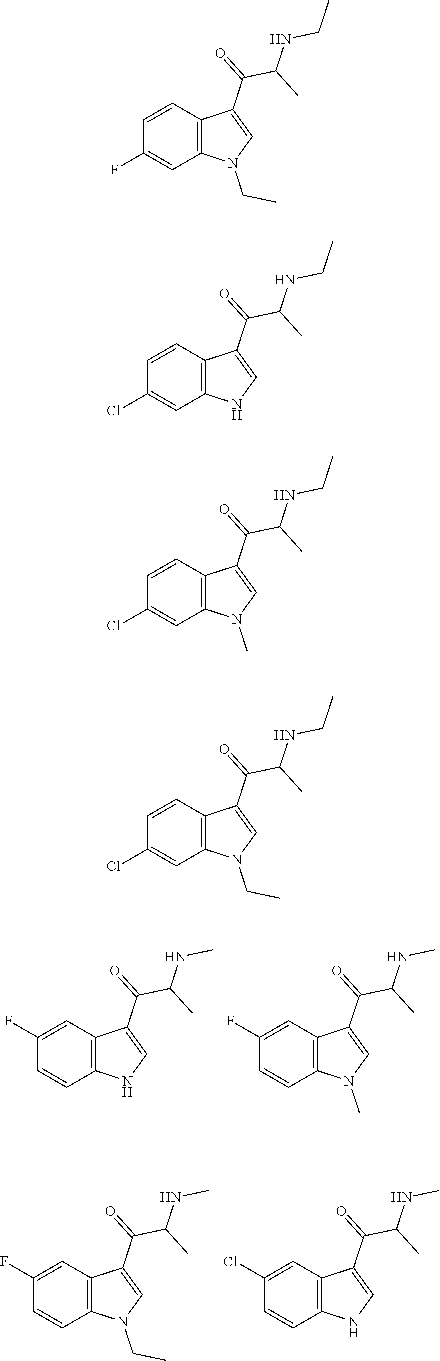

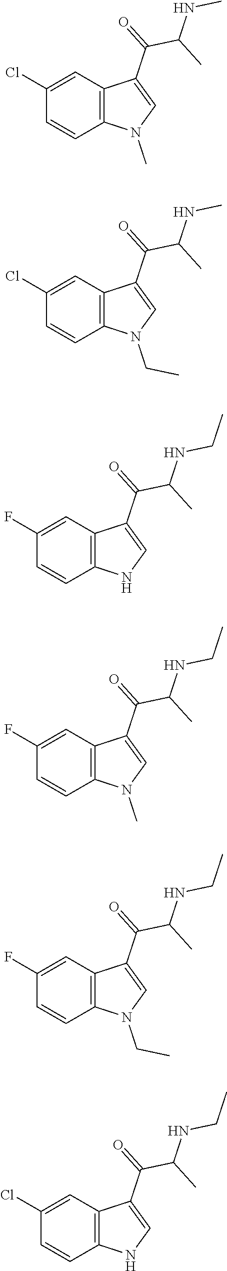 [SMILES: CNC(C)C(=O)c1cn(C)c2ccc(Cl)cc12 ; CCn1cc(C(=O)C(C)NC)c2cc(Cl)ccc21 ; CCNC(C)C(=O)c1c[nH]c2ccc(F)cc12 ; CCNC(C)C(=O)c1cn(C)c2ccc(F)cc12 ; CCNC(C)C(=O)c1cn(CC)c2ccc(F)cc12 ; CCNC(C)C(=O)c1c[nH]c2ccc(Cl)cc12]

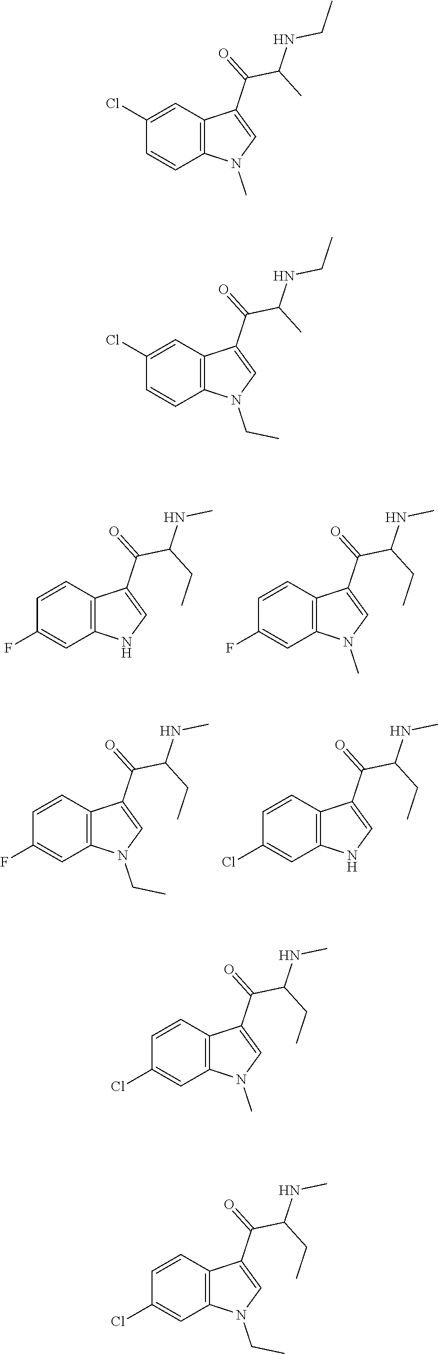

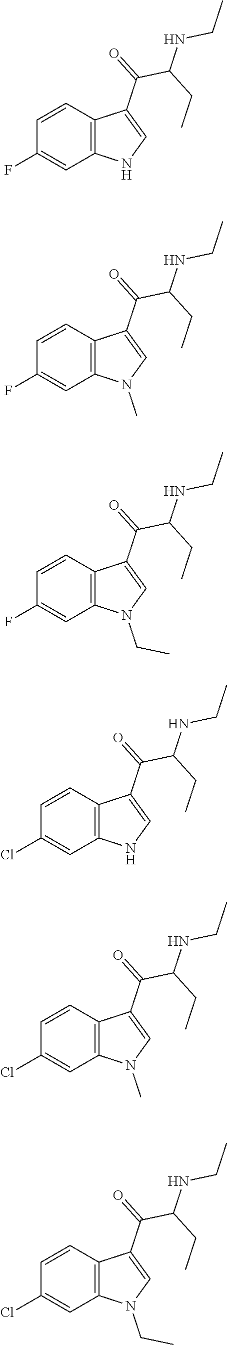 [SMILES: CCNC(CC)C(=O)c1c[nH]c2cc(F)ccc12 ; CCNC(CC)C(=O)c1cn(C)c2cc(F)ccc12 ; CCNC(CC)C(=O)c1cn(CC)c2cc(F)ccc12 ; CCNC(CC)C(=O)c1c[nH]c2cc(Cl)ccc12 ; CCNC(CC)C(=O)c1cn(C)c2cc(Cl)ccc12 ; CCNC(CC)C(=O)c1cn(CC)c2cc(Cl)ccc12]

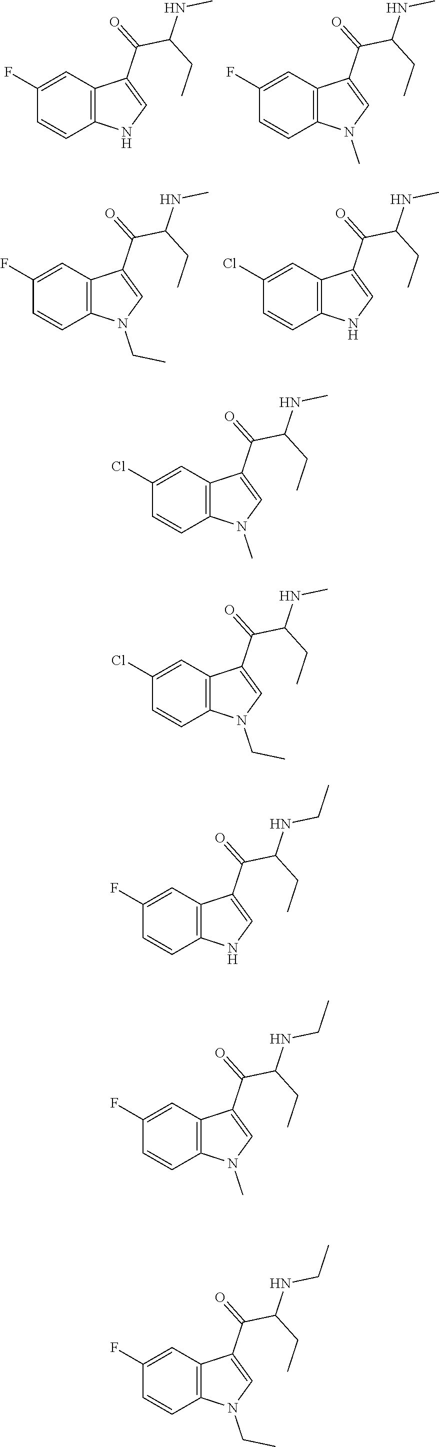

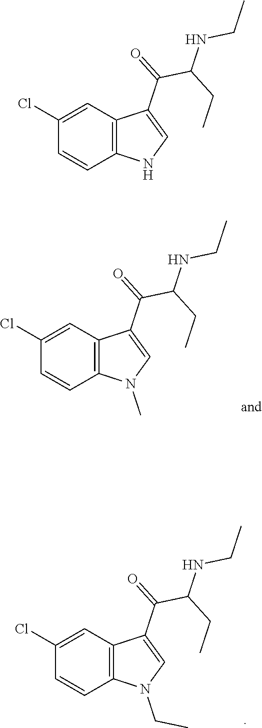 [SMILES: CCNC(CC)C(=O)c1c[nH]c2ccc(Cl)cc12 ; CCNC(CC)C(=O)c1cn(C)c2ccc(Cl)cc12 ; CCNC(CC)C(=O)c1cn(CC)c2ccc(Cl)cc12]

In certain embodiments the combination of the present invention includes a compound selected from:

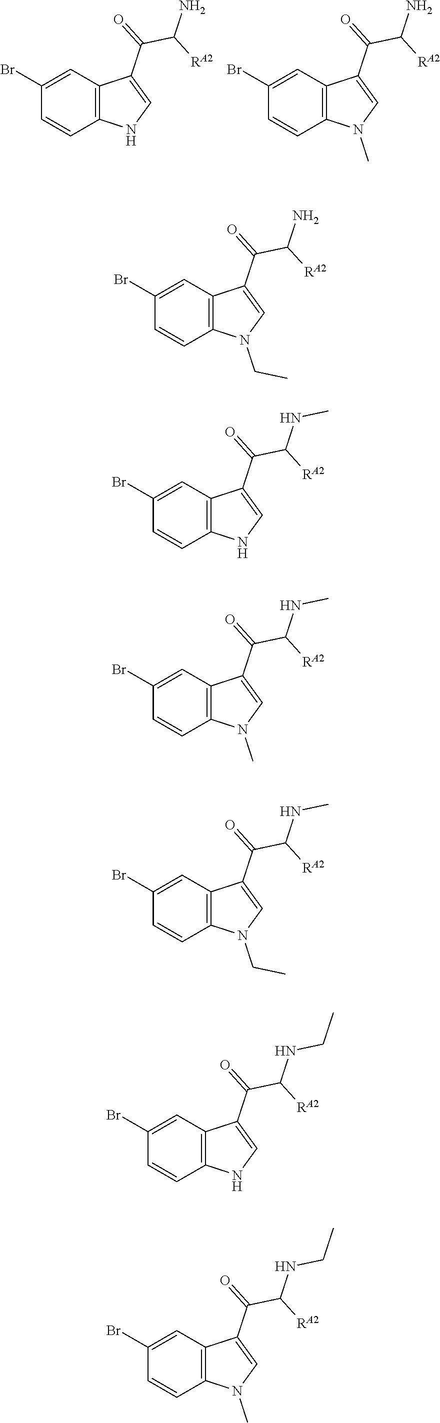

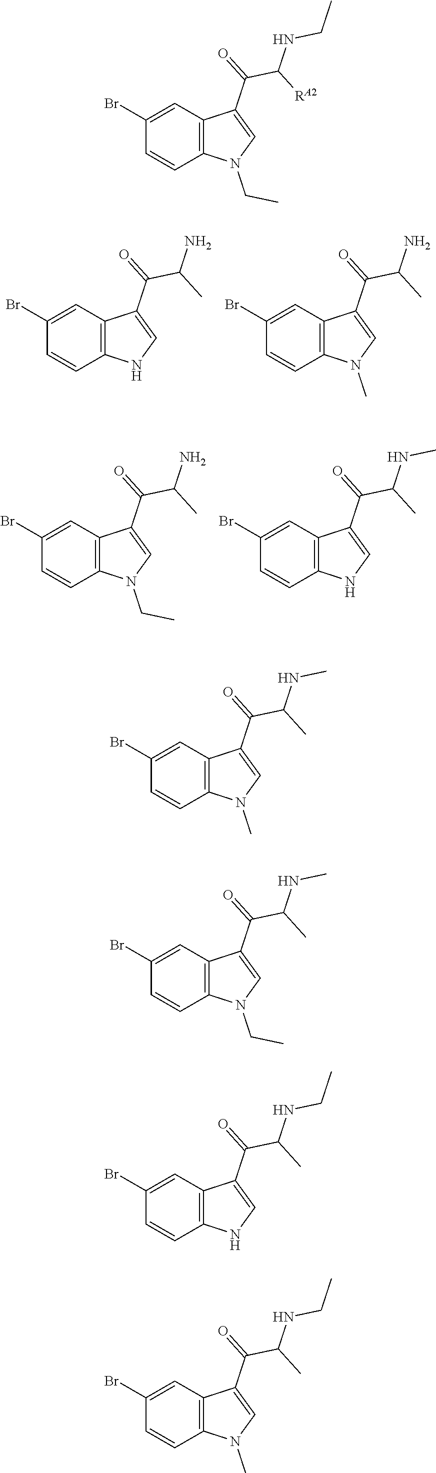

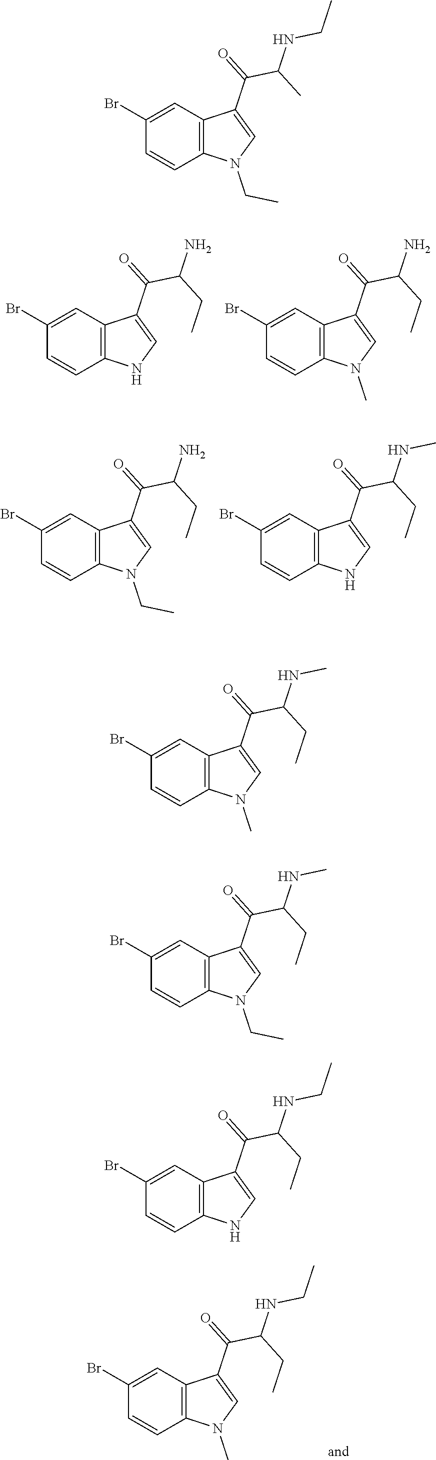

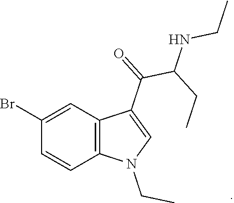 [SMILES: CCNC(CC)C(=O)c1cn(CC)c2ccc(Br)cc12]

In certain embodiments the combination of the present invention includes a compound selected from:

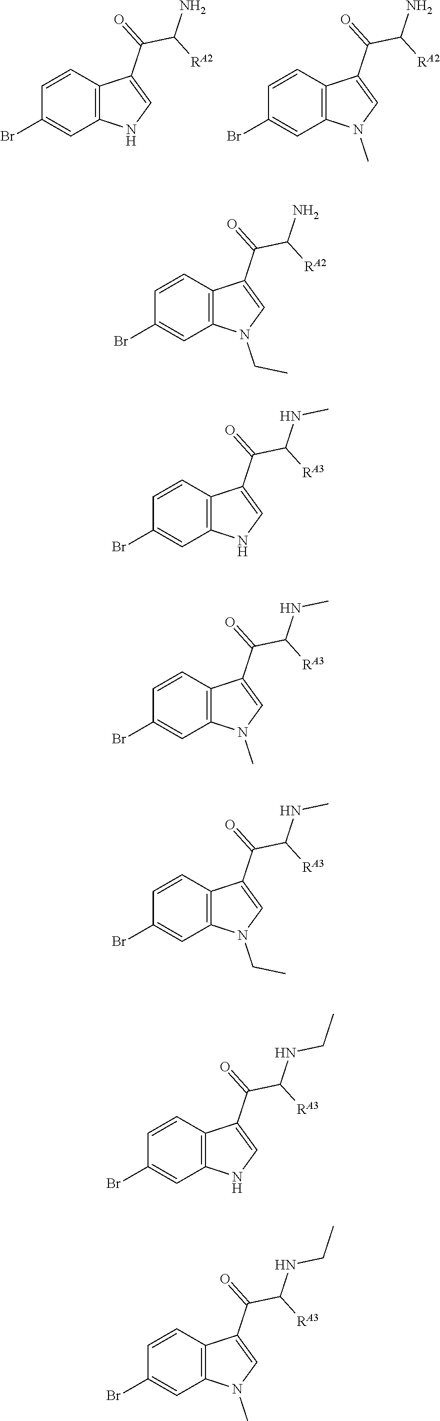

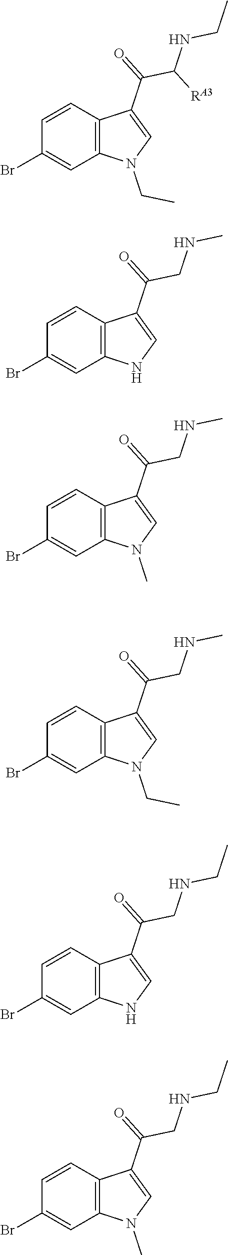

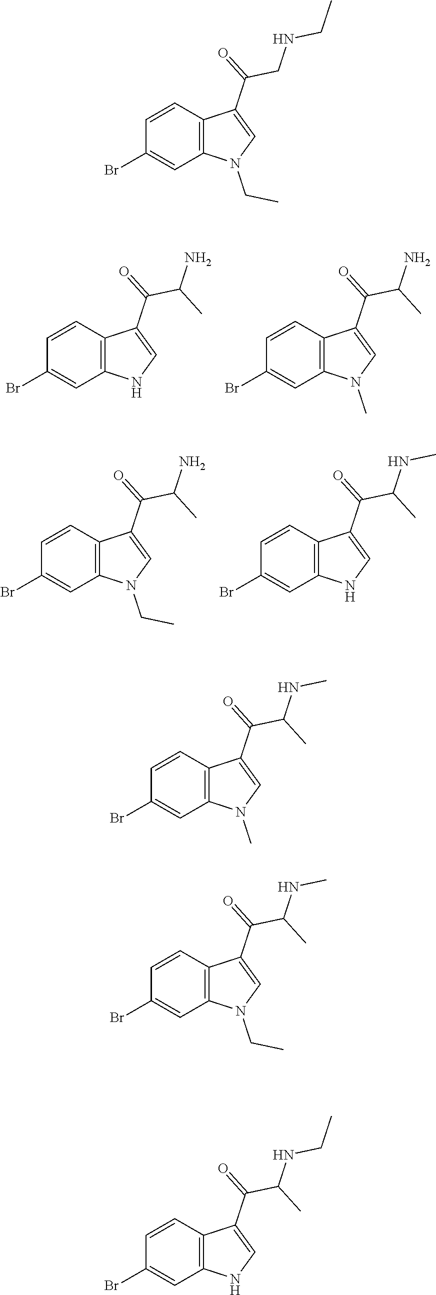

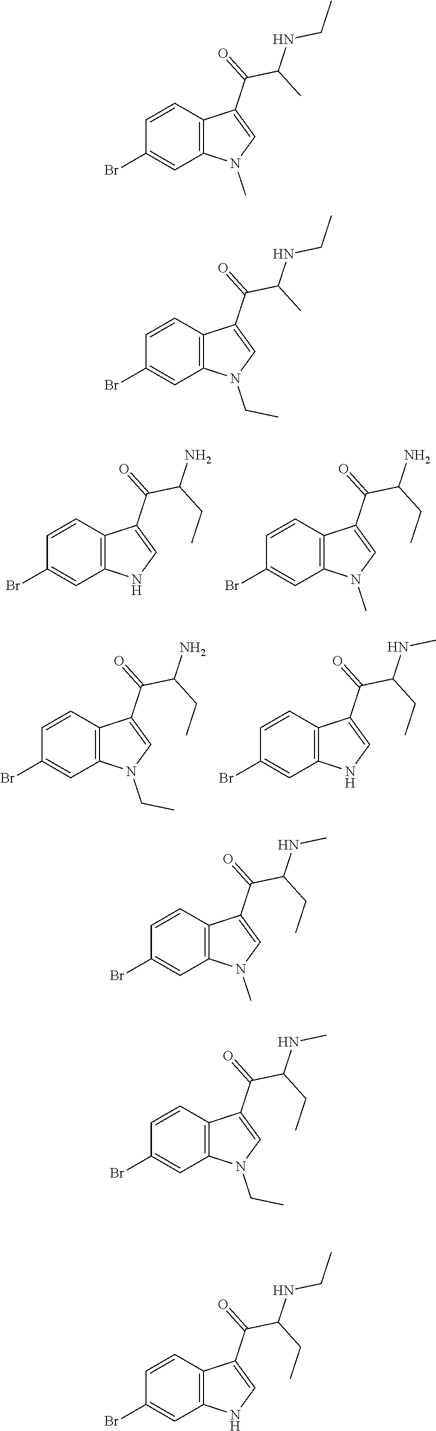

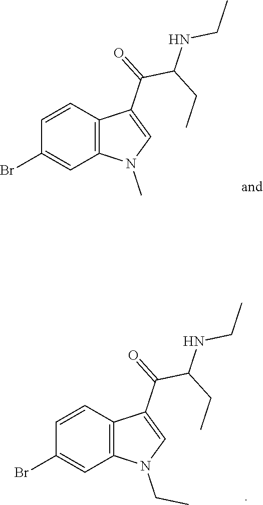 [SMILES: CCNC(CC)C(=O)c1cn(C)c2cc(Br)ccc12 ; CCNC(CC)C(=O)c1cn(CC)c2cc(Br)ccc12]

In certain embodiments the combination of the present invention includes a compound

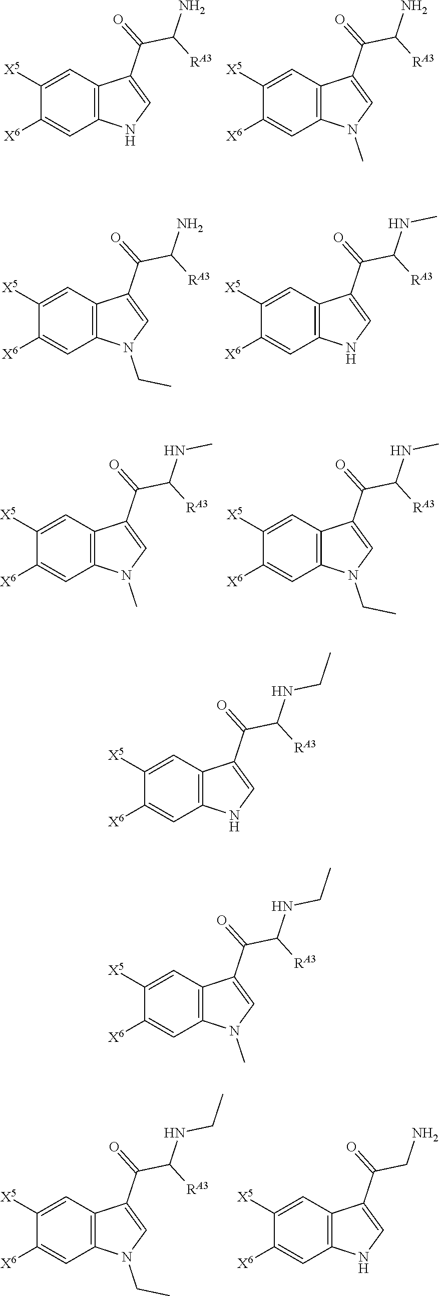

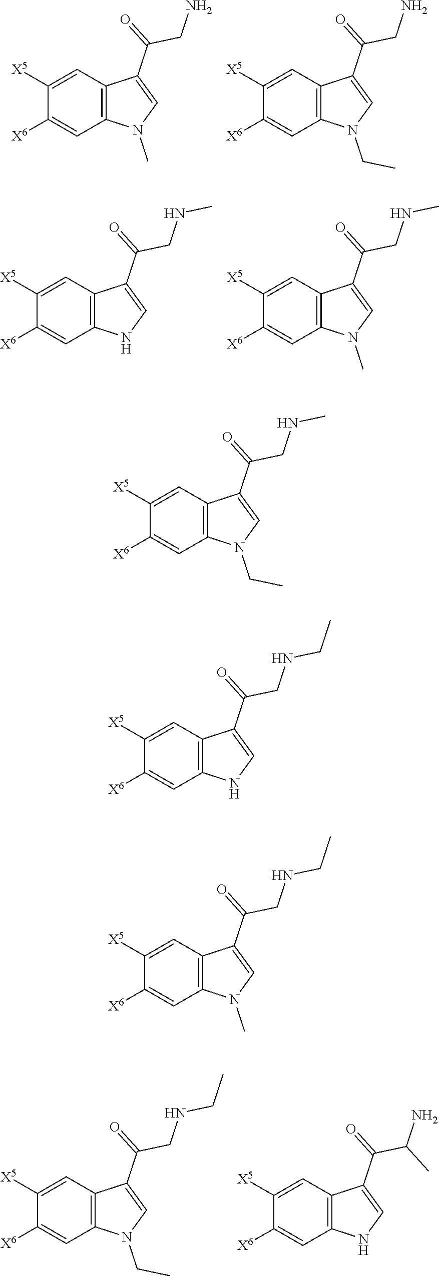

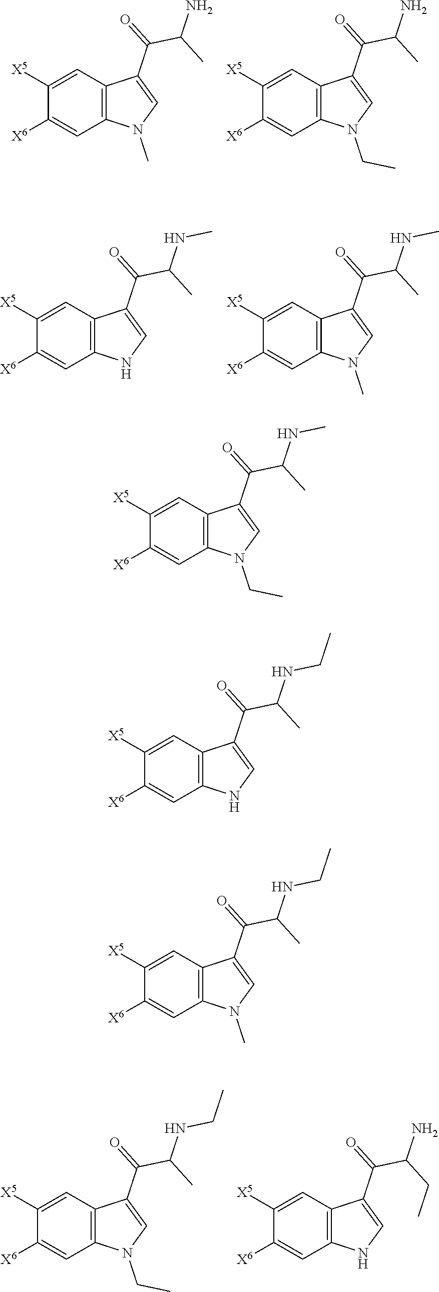

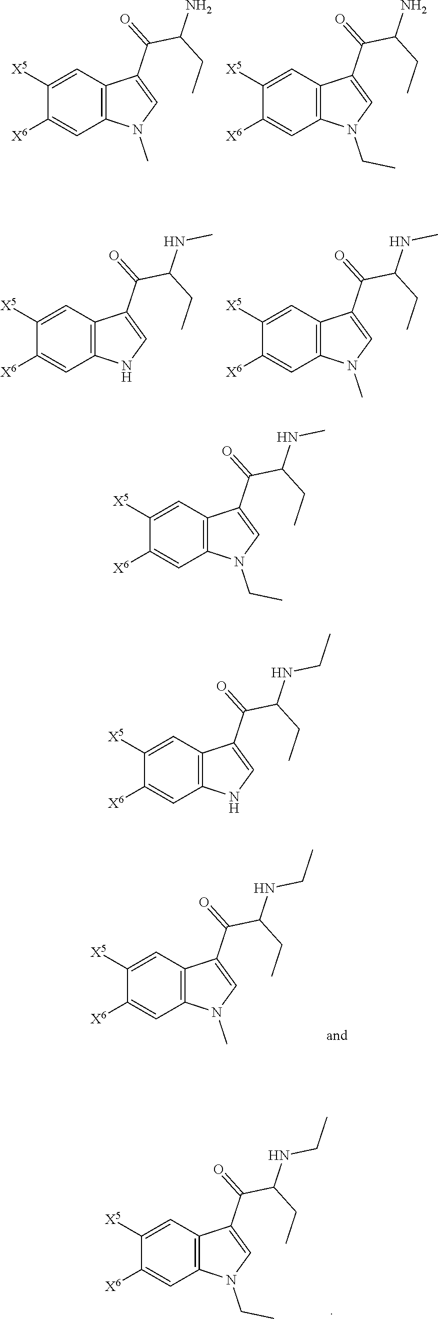

In certain embodiments the combination of the present invention includes a compound selected from:

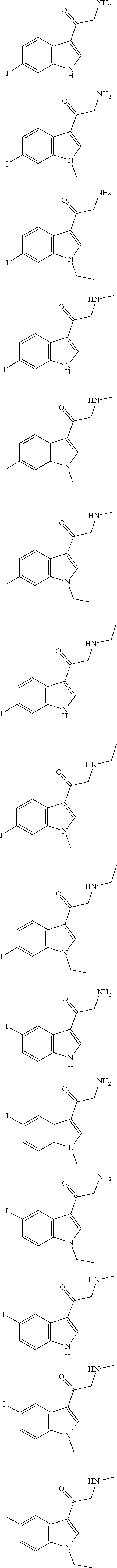

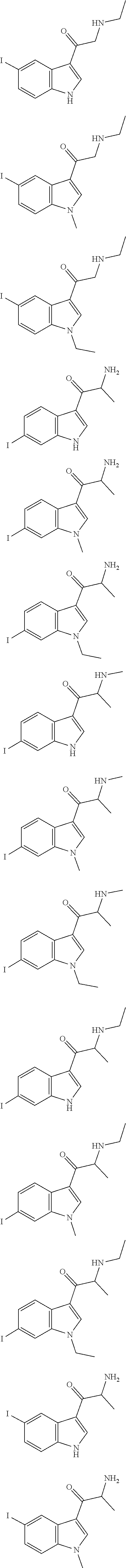

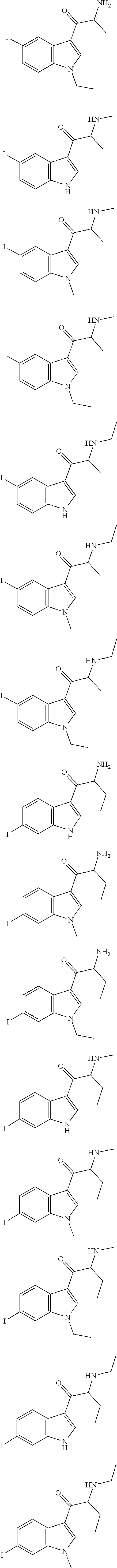

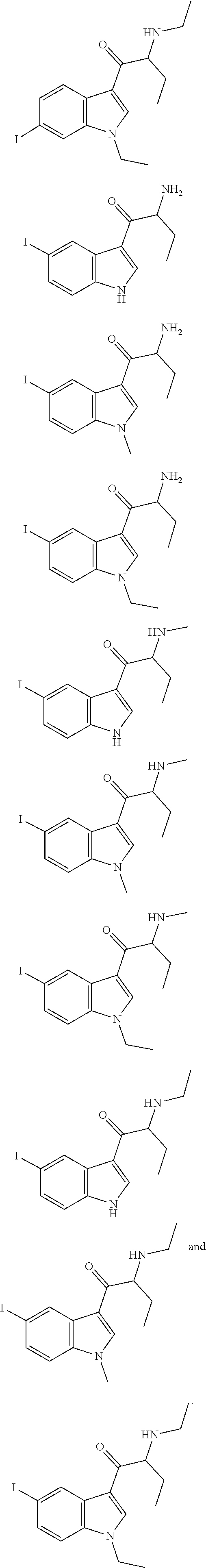

In certain embodiments the combination of the present invention includes a compound selected from:

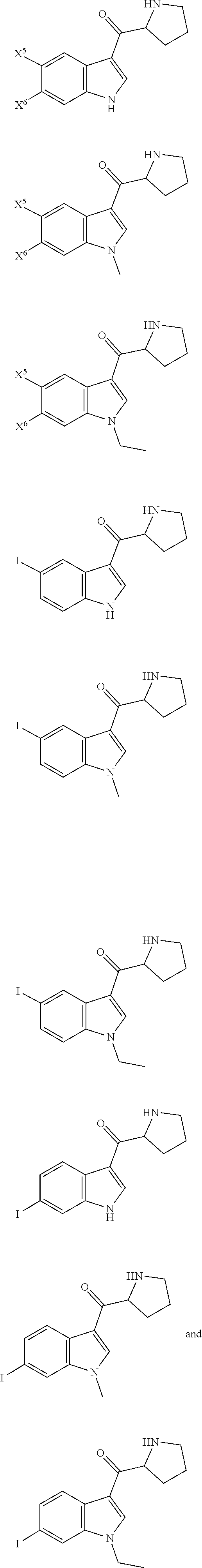

In certain embodiments the combination of the present invention includes a compound selected from:

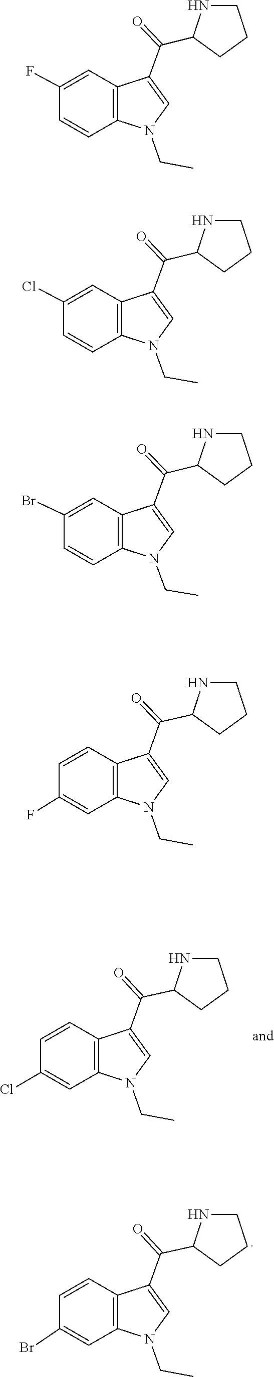 [SMILES: CCn1cc(C(=O)C2CCCN2)c2cc(F)ccc21 ; CCn1cc(C(=O)C2CCCN2)c2cc(Cl)ccc21 ; CCn1cc(C(=O)C2CCCN2)c2cc(Br)ccc21 ; CCn1cc(C(=O)C2CCCN2)c2ccc(F)cc21 ; CCn1cc(C(=O)C2CCCN2)c2ccc(Cl)cc21 ; CCn1cc(C(=O)C2CCCN2)c2ccc(Br)cc21]

In certain embodiments the combination of the present invention includes a compound selected from:

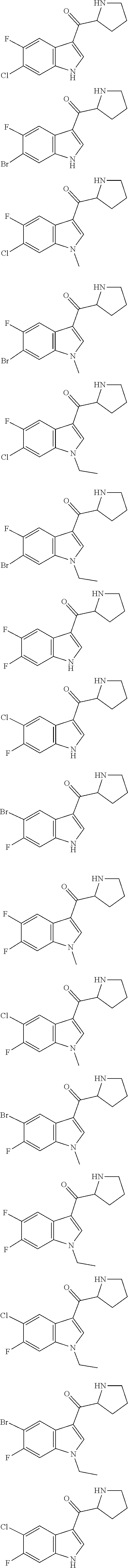

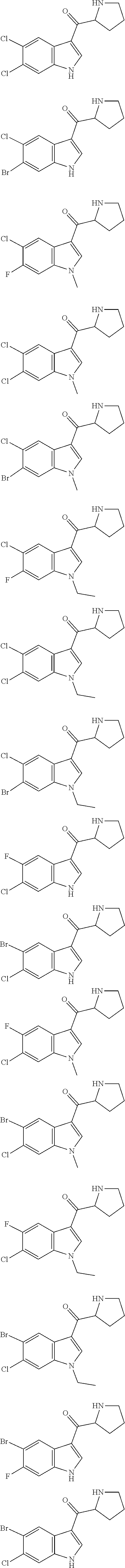

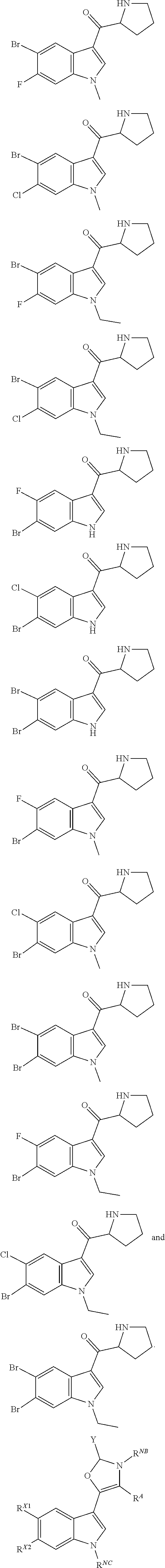

Exemplary R^{P} and R^{E} Groups

In some embodiments, an entactogen prodrug is provided for use in a mixture described herein. In some embodiments, the entactogen prodrug comprises at least one amino acid directly bonded to the entactogen. In some embodiments, the at least one amino acid is selected from Table 1. In some embodiments, the at least one amino acid comprises at least two amino acids as a peptide. In some embodiments, the at least two amino acids are a valine bonded to a valine via a peptide bond. In some embodiments, the at least two amino acids are three glycines bonded via peptide bonds.

In certain embodiments a basic amine of a compound described herein is substituted with an R^{P} group wherein the R^{P} group is a prodrug moiety for example an amino acid moiety. The following examples provide non-exhaustive illustrations of R^{P} contemplated in some embodiments. However, this table is used for illustrative purposes and other possibilities inherent in the definition of R^{P} are contemplated. Similarly, geometric and other isomers are also contemplated.

TABLE 1
Exemplary R^{P} Amino Acids and Peptides

| Example Number | Example R^{P} |
|---|---|
| R^{P}-1 | Gly |
| R^{P}-2 | Gly-Gly |
| R^{P}-3 | Gly-Gly-Gly |
| R^{P}-4 | Phe |
| R^{P}-5 | Trp |
| R^{P}-6 | Trp-Trp |
| R^{P}-7 | Met |
| R^{P}-8 | Met-Met |
| R^{P}-9 | Met-Met-Met |
| R^{P}-10 | N-acetyl-Met |
| R^{P}-11 | Cys |
| R^{P}-12 | Cys-Cys |
| R^{P}-13 | N-acetyl-Cys |
| R^{P}-14 | D-ribose-Cys |
| R^{P}-15 | His |
| R^{P}-16 | His-His |
| R^{P}-17 | Ala-His |
| R^{P}-18 | Arg |
| R^{P}-19 | Arg-Arg |
| R^{P}-20 | Tyr |
| R^{P}-21 | Tyr-Tyr |
| R^{P}-22 | Val |
| R^{P}-23 | Val-Val |

In some embodiments, a tryptamine of the present invention has one or more R^{E} moieties conjugated either directly to the tryptamine or to an R^{P} group that is directly bonded to the compound. Table 2 provides non-limiting illustrations of R^{E} contemplated in some embodiments. However, these are intended for illustrative purposes and other possibilities inherent in the definition of R^{E} are contemplated. Similarly, enantiomers and other stereoisomers are also contemplated.

TABLE 2
Example R^{E} Prodrug Groups

| R^{E} Number | R^{E} Prodrug Group |
|---|---|
| R^{E}-1 | 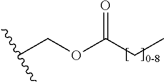 |
| R^{E}-2 | 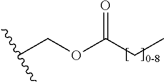 |
| R^{E}-3 | 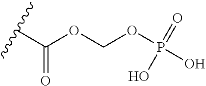 [SMILES: *C(=O)OCOP(=O)(O)O] |
| R^{E}-4 | 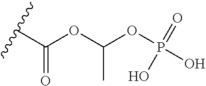 [SMILES: *C(=O)OC(C)OP(=O)(O)O] |
| R^{E}-5 | 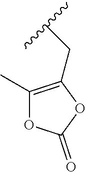 [SMILES: *Cc1oc(=O)oc1C] |
| R^{E}-6 | 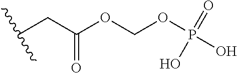 [SMILES: *CC(=O)OCOP(=O)(O)O] |
| R^{E}-7 | 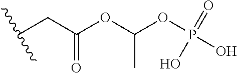 [SMILES: *CC(=O)OC(C)OP(=O)(O)O] |
| R^{E}-8 | 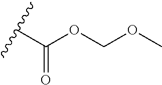 [SMILES: *C(=O)OCOC] |
| R^{E}-9 | 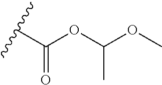 [SMILES: *C(=O)OC(C)OC] |
| R^{E}-10 | 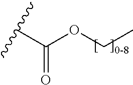 |
| R^{E}-11 | 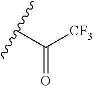 [SMILES: *C(=O)C(F)(F)F] |
| R^{E}-12 | 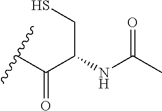 [SMILES: *C(=O)[C@H](CS)NC(C)=O] |
| R^{E}-13 | 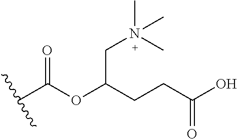 |
| R^{E}-14 | 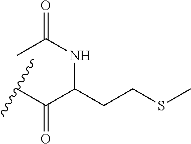 [SMILES: *C(=O)C(CCSC)NC(C)=O] |
| R^{E}-15 | 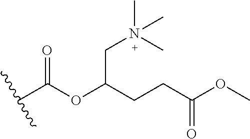 |
| R^{E}-16 | 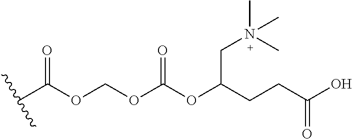 |
| R^{E}-17 | 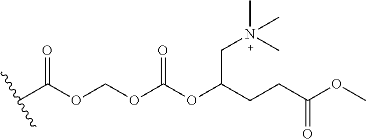 |
| R^{E}-18 | 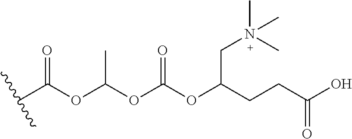 |
| R^{E}-19 | 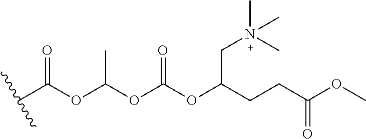 |
| R^{E}-20 | 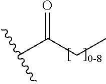 |
| R^{E}-21 | 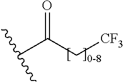 |
| R^{E}-22 | 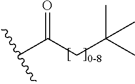 |
| R^{E}-23 | 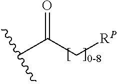 |
| R^{E}-24 | 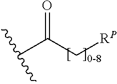 |

In some embodiments, the entactogen prodrug is selected from the non-limiting structures shown below (wherein the R^{E}-7n substituents refer to the examples R^{E}-1 through R^{E}-24 in Table 2 above).

TABLE 3
Example Compounds for use in the Present Invention

| | |
|---|---|
| 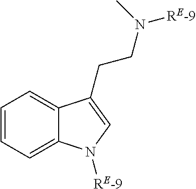 | P-1 |
| 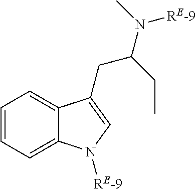 | P-2 |
| 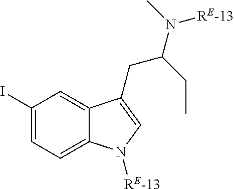 | P-3 |
| 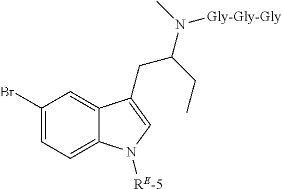 | P-4 |
| 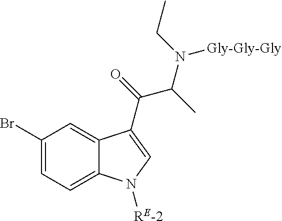 | P-5 |
| 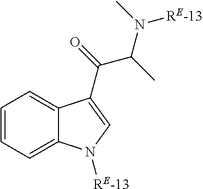 | P-6 |
| 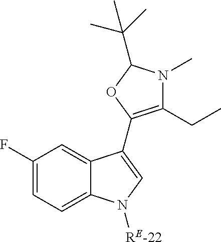 | P-7 |
| 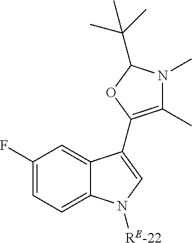 | P-8 |
| 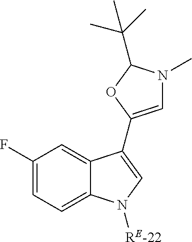 | P-9 |
| 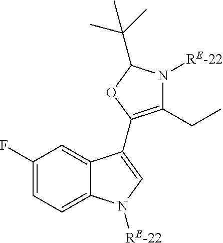 | P-10 |
| 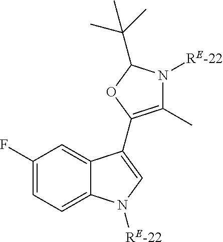 | P-11 |
| 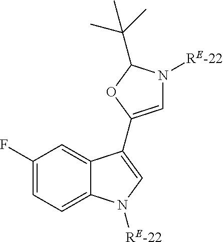 | P-12 |
| 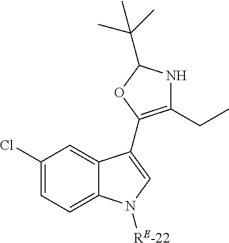 | P-13 |
| 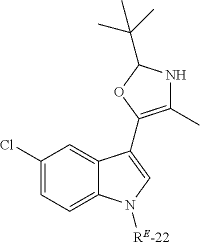 | P-14 |
| 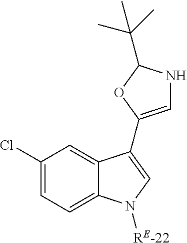 | P-15 |
| 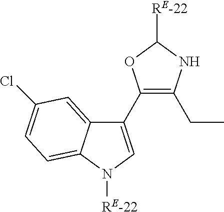 | P-16 |
| 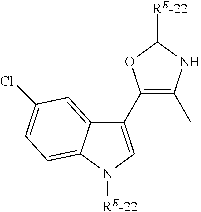 | P-17 |
| 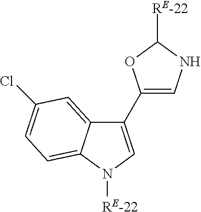 | P-18 |
| 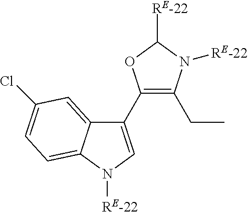 | P-19 |
| 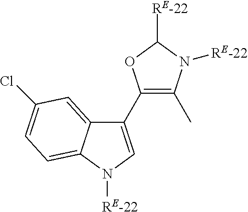 | P-20 |
| 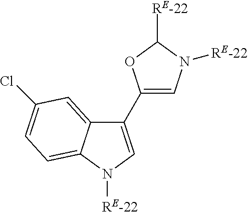 | P-21 |
| 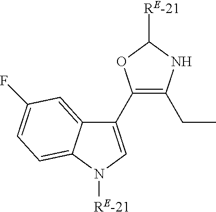 | P-22 |
| 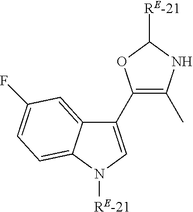 | P-23 |
| 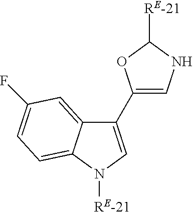 | P-24 |
| 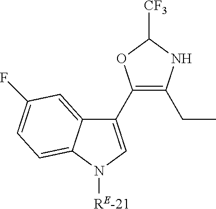 | P-25 |
| 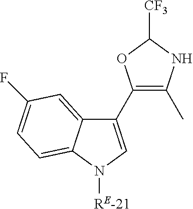 | P-26 |
| 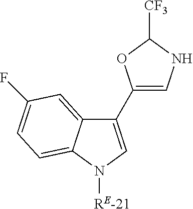 | P-27 |
| 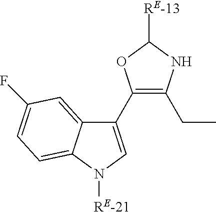 | P-28 |
| 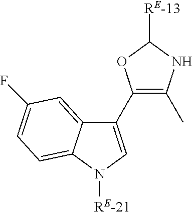 | P-29 |
| 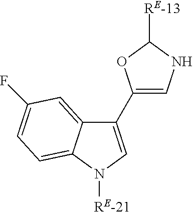 | P-30 |

In Table 4 below, Q indicates either oxygen or sulfur.

TABLE 4
Exemplary Compounds for Use in the Present Invention

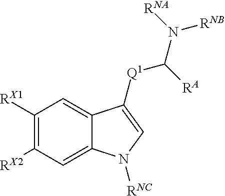

| Com- | | | | | | | |
|---|---|---|---|---|---|---|---|
| pound | R^{NA} | R^{NB} | R^{A} | Q¹ | R^{NC} | R^{X1} | R^{X2} |
| T-1 | CH₂CH₃ | CH₂CH₃ | CH₂CH₃ | Oxo | CH₃ | H | Halo |
| T-2 | CH₂CH₃ | CH₂CH₃ | CH₂CH₃ | Oxo | CH₃ | Halo | H |
| T-3 | CH₂CH₃ | CH₂CH₃ | CH₂CH₃ | Oxo | H | H | Halo |
| T-4 | CH₂CH₃ | CH₂CH₃ | CH₂CH₃ | Oxo | H | Halo | H |
| T-5 | CH₂CH₃ | CH₂CH₃ | CH₂CH₃ | Oxo | Y | H | Halo |
| T-6 | CH₂CH₃ | CH₂CH₃ | CH₂CH₃ | Oxo | Y | Halo | H |
| T-7 | CH₂CH₃ | CH₂CH₃ | CH₂CH₃ | H | CH₃ | H | H |
| T-8 | CH₂CH₃ | CH₂CH₃ | CH₂CH₃ | H | CH₃ | H | Halo |
| T-9 | CH₂CH₃ | CH₂CH₃ | CH₂CH₃ | H | CH₃ | H | QCH₂CH₃ |
| T-10 | CH₂CH₃ | CH₂CH₃ | CH₂CH₃ | H | CH₃ | H | QCH₃ |
| T-11 | CH₂CH₃ | CH₂CH₃ | CH₂CH₃ | H | CH₃ | Halo | H |
| T-12 | CH₂CH₃ | CH₂CH₃ | CH₂CH₃ | H | CH₃ | QCH₂CH₃ | H |
| T-13 | CH₂CH₃ | CH₂CH₃ | CH₂CH₃ | H | CH₃ | QCH₃ | H |
| T-14 | CH₂CH₃ | CH₂CH₃ | CH₃ | Oxo | CH₃ | H | Halo |
| T-15 | CH₂CH₃ | CH₂CH₃ | CH₃ | Oxo | CH₃ | Halo | H |
| T-16 | CH₂CH₃ | CH₂CH₃ | CH₃ | Oxo | H | H | Halo |
| T-17 | CH₂CH₃ | CH₂CH₃ | CH₃ | Oxo | H | Halo | H |
| T-18 | CH₂CH₃ | CH₂CH₃ | CH₃ | Oxo | Y | H | Halo |
| T-19 | CH₂CH₃ | CH₂CH₃ | CH₃ | Oxo | Y | Halo | H |
| T-20 | CH₂CH₃ | CH₂CH₃ | CH₃ | H | CH₃ | H | H |
| T-21 | CH₂CH₃ | CH₂CH₃ | CH₃ | H | CH₃ | H | Halo |
| T-22 | CH₂CH₃ | CH₂CH₃ | CH₃ | H | CH₃ | H | QCH₂CH₃ |
| T-23 | CH₂CH₃ | CH₂CH₃ | CH₃ | H | CH₃ | H | QCH₃ |
| T-24 | CH₂CH₃ | CH₂CH₃ | CH₃ | H | CH₃ | Halo | H |
| T-25 | CH₂CH₃ | CH₂CH₃ | CH₃ | H | CH₃ | QCH₂CH₃ | H |
| T-26 | CH₂CH₃ | CH₂CH₃ | CH₃ | H | CH₃ | QCH₃ | H |
| T-27 | CH₂CH₃ | CH₂CH₃ | H | Oxo | CH₃ | H | Halo |
| T-28 | CH₂CH₃ | CH₂CH₃ | H | Oxo | CH₃ | Halo | H |
| T-29 | CH₂CH₃ | CH₂CH₃ | H | Oxo | H | H | Halo |
| T-30 | CH₂CH₃ | CH₂CH₃ | H | Oxo | H | Halo | H |
| T-31 | CH₂CH₃ | CH₂CH₃ | H | Oxo | Y | H | Halo |
| T-32 | CH₂CH₃ | CH₂CH₃ | H | Oxo | Y | Halo | H |
| T-33 | CH₂CH₃ | CH₂CH₃ | H | H | CH₃ | H | H |
| T-34 | CH₂CH₃ | CH₂CH₃ | H | H | CH₃ | H | Halo |
| T-35 | CH₂CH₃ | CH₂CH₃ | H | H | CH | H | QCH₂CH₃ |
| T-36 | CH₂CH₃ | CH₂CH₃ | H | H | CH₃ | H | QCH₃ |
| T-37 | CH₂CH₃ | CH₂CH₃ | H | H | CH₃ | Halo | H |
| T-38 | CH₂CH₃ | CH₂CH₃ | H | H | CH₃ | QCH₂CH₃ | H |
| T-39 | CH₂CH₃ | CH₂CH₃ | H | H | CH₃ | QCH₃ | H |
| T-40 | CH₃ | CH₃ | CH₂CH₃ | Oxo | CH₃ | H | Halo |
| T-41 | CH₃ | CH₃ | CH₂CH₃ | Oxo | CH₃ | Halo | H |
| T-42 | CH₃ | CH₃ | CH₂CH₃ | Oxo | H | H | Halo |
| T-43 | CH₃ | CH₃ | CH₂CH₃ | Oxo | H | Halo | H |
| T-44 | CH₃ | CH₃ | CH₂CH₃ | Oxo | Y | H | Halo |
| T-45 | CH₃ | CH₃ | CH₂CH₃ | Oxo | Y | Halo | H |
| T-46 | CH₃ | CH₃ | CH₂CH₃ | H | CH₃ | H | H |
| T-47 | CH₃ | CH₃ | CH₂CH₃ | H | CH₃ | H | Halo |
| T-48 | CH₃ | CH₃ | CH₂CH₃ | H | CH₃ | H | QCH₂CH₃ |
| T-49 | CH₃ | CH₃ | CH₂CH₃ | H | CH₃ | H | QCH₃ |
| T-50 | CH₃ | CH₃ | CH₂CH₃ | H | CH₃ | Halo | H |
| T-51 | CH₃ | CH₃ | CH₂CH₃ | H | CH₃ | QCH₂CH₃ | H |
| T-52 | CH₃ | CH₃ | CH₂CH₃ | H | CH₃ | QCH₃ | H |
| T-53 | CH₃ | CH₃ | CH₃ | Oxo | CH₃ | H | Halo |
| T-54 | CH₃ | CH₃ | CH₃ | Oxo | CH₃ | Halo | H |
| T-55 | CH₃ | CH₃ | CH₃ | Oxo | H | H | Halo |
| T-56 | CH₃ | CH₃ | CH₃ | Oxo | H | Halo | H |
| T-57 | CH₃ | CH₃ | CH₃ | Oxo | Y | H | Halo |
| T-58 | CH₃ | CH₃ | CH₃ | Oxo | Y | Halo | H |
| T-59 | CH₃ | CH₃ | CH | H | CH₃ | H | H |
| T-60 | CH₃ | CH₃ | CH₃ | H | CH₃ | H | Halo |
| T-61 | CH₃ | CH₃ | CH₃ | H | CH₃ | H | QCH₂CH₃ |
| T-62 | CH₃ | CH₃ | CH₃ | H | CH₃ | H | QCH₃ |
| T-63 | CH₃ | CH₃ | CH₃ | H | CH₃ | Halo | H |
| T-64 | CH₃ | CH₃ | CH₃ | H | CH₃ | QCH₂CH₃ | H |
| T-65 | CH₃ | CH₃ | CH₃ | H | CH₃ | QCH₃ | H |
| T-66 | CH₃ | CH₃ | H | Oxo | CH₃ | H | Halo |
| T-67 | CH₃ | CH₃ | H | Oxo | CH₃ | Halo | H |
| T-68 | CH₃ | CH₃ | H | Oxo | H | H | Halo |
| T-69 | CH₃ | CH₃ | H | Oxo | H | Halo | H |
| T-70 | CH₃ | CH₃ | H | Oxo | Y | H | Halo |
| T-71 | CH₃ | CH₃ | H | Oxo | Y | Halo | H |
| T-72 | H | CH₂CH₃ | CH₂CH₃ | Oxo | CH₃ | H | Halo |
| T-73 | H | CH₂CH₃ | CH₂CH₃ | Oxo | CH₃ | Halo | H |
| T-74 | H | CH₂CH₃ | CH₂CH₃ | Oxo | H | H | Halo |
| T-75 | H | CH₂CH₃ | CH₂CH₃ | Oxo | H | Halo | H |
| T-76 | H | CH₂CH₃ | CH₂CH₃ | Oxo | Y | H | H |
| T-77 | H | CH₂CH₃ | CH₂CH₃ | Oxo | Y | H | Halo |
| T-78 | H | CH₂CH₃ | CH₂CH₃ | Oxo | Y | H | QCH₂CH₃ |
| T-79 | H | CH₂CH₃ | CH₂CH₃ | Oxo | Y | H | QCH₃ |
| T-80 | H | CH₂CH₃ | CH₂CH₃ | Oxo | Y | Halo | H |
| T-81 | H | CH₂CH₃ | CH₂CH₃ | Oxo | Y | QCH₂CH₃ | H |
| T-82 | H | CH₂CH₃ | CH₂CH₃ | Oxo | Y | QCH₃ | H |
| T-83 | H | CH₂CH₃ | CH₂CH₃ | H | CH₃ | H | H |
| T-84 | H | CH₂CH₃ | CH₂CH₃ | H | CH₃ | H | Halo |
| T-85 | H | CH₂CH₃ | CH₂CH₃ | H | CH₃ | H | QCH₂CH₃ |
| T-86 | H | CH₂CH₃ | CH₂CH₃ | H | CH₃ | H | QCH₃ |
| T-87 | H | CH₂CH₃ | CH₂CH₃ | H | CH₃ | Halo | H |
| T-88 | H | CH₂CH₃ | CH₂CH₃ | H | CH | QCH₂CH₃ | H |
| T-89 | H | CH₂CH₃ | CH₂CH₃ | H | CH₃ | QCH₃ | H |
| T-90 | H | CH₂CH₃ | CH₂CH₃ | H | Y | H | H |
| T-91 | H | CH₂CH₃ | CH₂CH₃ | H | Y | H | Halo |
| T-92 | H | CH₂CH₃ | CH₂CH₃ | H | Y | H | QCH₂CH₃ |
| T-93 | H | CH₂CH₃ | CH₂CH₃ | H | Y | H | QCH₃ |
| T-94 | H | CH₂CH₃ | CH₂CH₃ | H | Y | Halo | H |
| T-95 | H | CH₂CH₃ | CH₂CH₃ | H | Y | QCH₂CH₃ | H |
| T-96 | H | CH₂CH₃ | CH₂CH₃ | H | Y | QCH₃ | H |
| T-97 | H | CH₂CH₃ | CH₃ | Oxo | CH₃ | H | Halo |
| T-98 | H | CH₂CH₃ | CH₃ | Oxo | CH₃ | Halo | H |
| T-99 | H | CH₂CH₃ | CH₃ | Oxo | H | H | Halo |
| T-100 | H | CH₂CH₃ | CH₃ | Oxo | H | Halo | H |
| T-101 | H | CH₂CH₃ | CH₃ | Oxo | Y | H | H |
| T-102 | H | CH₂CH₃ | CH₃ | Oxo | Y | H | Halo |
| T-103 | H | CH₂CH₃ | CH₃ | Oxo | Y | H | QCH₂CH₃ |
| T-104 | H | CH₂CH₃ | CH₃ | Oxo | Y | H | QCH₃ |
| T-105 | H | CH₂CH₃ | CH₃ | Oxo | Y | Halo | H |
| T-106 | H | CH₂CH₃ | CH₃ | Oxo | Y | QCH₂CH₃ | H |
| T-107 | H | CH₂CH₃ | CH₃ | Oxo | Y | QCH₃ | H |
| T-108 | H | CH₂CH₃ | CH₃ | H | CH₃ | H | H |
| T-109 | H | CH₂CH₃ | CH₃ | H | CH₃ | H | Halo |
| T-110 | H | CH₂CH₃ | CH₃ | H | CH₃ | H | QCH₂CH₃ |
| T-111 | H | CH₂CH₃ | CH₃ | H | CH₃ | H | QCH₃ |
| T-112 | H | CH₂CH₃ | CH₃ | H | CH₃ | Halo | H |
| T-113 | H | CH₂CH₃ | CH₃ | H | CH₃ | QCH₂CH₃ | H |
| T-114 | H | CH₂CH₃ | CH₃ | H | CH₃ | QCH₃ | H |
| T-115 | H | CH₂CH₃ | CH₃ | H | Y | H | H |
| T-116 | H | CH₂CH₃ | CH₃ | H | Y | H | Halo |
| T-117 | H | CH₂CH₃ | CH₃ | H | Y | H | QCH₂CH₃ |
| T-118 | H | CH₂CH₃ | CH₃ | H | Y | H | QCH₃ |
| T-119 | H | CH₂CH₃ | CH₃ | H | Y | Halo | H |
| T-120 | H | CH₂CH₃ | CH₃ | H | Y | QCH₂CH₃ | H |
| T-121 | H | CH₂CH₃ | CH₃ | H | Y | QCH₃ | H |
| T-122 | H | CH₂CH₃ | H | Oxo | CH₃ | H | Halo |
| T-123 | H | CH₂CH₃ | H | Oxo | CH₃ | Halo | H |
| T-124 | H | CH₂CH₃ | H | Oxo | H | H | Halo |
| T-125 | H | CH₂CH₃ | H | Oxo | H | Halo | H |
| T-126 | H | CH₂CH₃ | H | Oxo | Y | H | H |
| T-127 | H | CH₂CH₃ | H | Oxo | Y | H | Halo |
| T-128 | H | CH₂CH₃ | H | Oxo | Y | H | QCH₂CH₃ |
| T-129 | H | CH₂CH₃ | H | Oxo | Y | H | QCH₃ |
| T-130 | H | CH₂CH₃ | H | Oxo | Y | Halo | H |
| T-131 | H | CH₂CH₃ | H | Oxo | Y | QCH₂CH₃ | H |
| T-132 | H | CH₂CH₃ | H | Oxo | Y | QCH₃ | H |
| T-133 | H | CH₂CH₃ | H | H | CH₃ | H | H |
| T-134 | H | CH₂CH₃ | H | H | CH₃ | H | Halo |
| T-135 | H | CH₂CH₃ | H | H | CH₃ | H | QCH₂CH₃ |
| T-136 | H | CH₂CH₃ | H | H | CH₃ | H | QCH₃ |
| T-137 | H | CH₂CH₃ | H | H | CH₃ | Halo | H |
| T-138 | H | CH₂CH₃ | H | H | CH₃ | QCH₂CH₃ | H |
| T-139 | H | CH₂CH₃ | H | H | CH | QCH₃ | H |
| T-140 | H | CH₂CH₃ | H | H | Y | H | H |
| T-141 | H | CH₂CH₃ | H | H | Y | H | QCH₂CH₃ |
| T-142 | H | CH₂CH₃ | H | H | Y | H | QCH₃ |
| T-143 | H | CH₂CH₃ | H | H | Y | QCH₂CH₃ | H |
| T-144 | H | CH₂CH₃ | H | H | Y | QCH₃ | H |
| T-145 | H | CH₃ | CH₂CH₃ | Oxo | CH₃ | H | Halo |
| T-146 | H | CH₃ | CH₂CH₃ | Oxo | CH₃ | Halo | H |
| T-147 | H | CH₃ | CH₂CH₃ | Oxo | H | H | Halo |
| T-148 | H | CH₃ | CH₂CH₃ | Oxo | H | Halo | H |
| T-149 | H | CH₃ | CH₂CH₃ | Oxo | Y | H | H |
| T-150 | H | CH₃ | CH₂CH₃ | Oxo | Y | H | Halo |
| T-151 | H | CH₃ | CH₂CH₃ | Oxo | Y | H | QCH₂CH₃ |
| T-152 | H | CH₃ | CH₂CH₃ | Oxo | Y | H | QCH₃ |
| T-153 | H | CH₃ | CH₂CH₃ | Oxo | Y | Halo | H |
| T-154 | H | CH₃ | CH₂CH₃ | Oxo | Y | QCH₂CH₃ | H |
| T-155 | H | CH₃ | CH₂CH₃ | Oxo | Y | QCH₃ | H |
| T-156 | H | CH₃ | CH₂CH₃ | H | CH₃ | H | H |
| T-157 | H | CH₃ | CH₂CH₃ | H | CH₃ | H | Halo |
| T-158 | H | CH₃ | CH₂CH₃ | H | CH₃ | H | QCH₂CH₃ |
| T-159 | H | CH₃ | CH₂CH₃ | H | CH₃ | H | QCH₃ |
| T-160 | H | CH₃ | CH₂CH₃ | H | CH₃ | Halo | H |
| T-161 | H | CH₃ | CH₂CH₃ | H | CH₃ | QCH₂CH₃ | H |
| T-162 | H | CH₃ | CH₂CH₃ | H | CH₃ | QCH₃ | H |
| T-163 | H | CH₃ | CH₂CH₃ | H | Y | H | H |
| T-164 | H | CH₃ | CH₂CH₃ | H | Y | H | Halo |
| T-165 | H | CH₃ | CH₂CH₃ | H | Y | H | QCH₂CH₃ |
| T-166 | H | CH₃ | CH₂CH₃ | H | Y | H | QCH₃ |
| T-167 | H | CH₃ | CH₂CH₃ | H | Y | Halo | H |
| T-168 | H | CH₃ | CH₂CH₃ | H | Y | QCH₂CH₃ | H |
| T-169 | H | CH₃ | CH₂CH₃ | H | Y | QCH₃ | H |
| T-170 | H | CH₃ | CH₃ | Oxo | CH₃ | H | Halo |
| T-171 | H | CH₃ | CH₃ | Oxo | CH₃ | Halo | H |
| T-172 | H | CH₃ | CH₃ | Oxo | H | H | Halo |
| T-173 | H | CH₃ | CH₃ | Oxo | H | Halo | H |
| T-174 | H | CH₃ | CH₃ | Oxo | Y | H | H |
| T-175 | H | CH₃ | CH₃ | Oxo | Y | H | Halo |
| T-176 | H | CH₃ | CH₃ | Oxo | Y | H | QCH₂CH₃ |
| T-177 | H | CH₃ | CH₃ | Oxo | Y | H | QCH₃ |
| T-178 | H | CH₃ | CH₃ | Oxo | Y | Halo | H |
| T-179 | H | CH₃ | CH₃ | Oxo | Y | QCH₂CH₃ | H |
| T-180 | H | CH₃ | CH₃ | Oxo | Y | QCH₃ | H |
| T-181 | H | CH₃ | CH₃ | H | CH₃ | H | H |
| T-182 | H | CH₃ | CH₃ | H | CH₃ | H | Halo |
| T-183 | H | CH₃ | CH₃ | H | CH₃ | H | QCH₂CH₃ |
| T-184 | H | CH₃ | CH₃ | H | CH₃ | H | QCH₃ |
| T-185 | H | CH₃ | CH₃ | H | CH₃ | Halo | H |
| T-186 | H | CH₃ | CH₃ | H | CH₃ | QCH₂CH₃ | H |
| T-187 | H | CH₃ | CH₃ | H | CH₃ | QCH₃ | H |
| T-188 | H | CH₃ | CH₃ | H | Y | H | H |
| T-189 | H | CH₃ | CH₃ | H | Y | H | Halo |
| T-190 | H | CH₃ | CH₃ | H | Y | H | QCH₂CH₃ |
| T-191 | H | CH₃ | CH₃ | H | Y | H | QCH₃ |
| T-192 | H | CH₃ | CH₃ | H | Y | Halo | H |
| T-193 | H | CH₃ | CH₃ | H | Y | QCH₂CH₃ | H |
| T-194 | H | CH₃ | CH₃ | H | Y | QCH₃ | H |
| T-195 | H | CH₃ | H | Oxo | CH₃ | H | Halo |
| T-196 | H | CH₃ | H | Oxo | CH₃ | Halo | H |
| T-197 | H | CH₃ | H | Oxo | H | H | Halo |
| T-198 | H | CH₃ | H | Oxo | H | Halo | H |
| T-199 | H | CH₃ | H | Oxo | Y | H | H |
| T-200 | H | CH₃ | H | Oxo | Y | H | Halo |
| T-201 | H | CH₃ | H | Oxo | Y | H | QCH₂CH₃ |
| T-202 | H | CH₃ | H | Oxo | Y | H | QCH₃ |
| T-203 | H | CH₃ | H | Oxo | Y | Halo | H |
| T-204 | H | CH₃ | H | Oxo | Y | QCH₂CH₃ | H |
| T-205 | H | CH₃ | H | Oxo | Y | QCH₃ | H |
| T-206 | H | CH₃ | H | H | CH₃ | H | H |
| T-207 | H | CH₃ | H | H | CH₃ | H | Halo |
| T-208 | H | CH₃ | H | H | CH₃ | H | QCH₂CH₃ |
| T-209 | H | CH₃ | H | H | CH₃ | H | QCH₃ |
| T-210 | H | CH₃ | H | H | CH₃ | Halo | H |
| T-211 | H | CH₃ | H | H | CH₃ | QCH₂CH₃ | H |
| T-212 | H | CH₃ | H | H | CH₃ | QCH₃ | H |
| T-213 | H | CH₃ | H | H | Y | H | H |
| T-214 | H | CH₃ | H | H | Y | H | QCH₂CH₃ |
| T-215 | H | CH₃ | H | H | Y | H | QCH₃ |
| T-216 | H | CH₃ | H | H | Y | QCH₂CH₃ | H |
| T-217 | H | CH₃ | H | H | Y | QCH₃ | H |
| T-218 | H | H | CH₂CH₃ | Oxo | CH₃ | H | Halo |
| T-219 | H | H | CH₂CH₃ | Oxo | CH₃ | H | QCH₃ |
| T-220 | H | H | CH₂CH₃ | Oxo | CH₃ | Halo | H |
| T-221 | H | H | CH₂CH₃ | Oxo | CH₃ | QCH₃ | H |
| T-222 | H | H | CH₂CH₃ | Oxo | H | H | Halo |
| T-223 | H | H | CH₂CH₃ | Oxo | H | Halo | H |
| T-224 | H | H | CH₂CH₃ | Oxo | Y | H | H |
| T-225 | H | H | CH₂CH₃ | Oxo | Y | H | Halo |
| T-226 | H | H | CH₂CH₃ | Oxo | Y | H | QCH₂CH₃ |
| T-227 | H | H | CH₂CH₃ | Oxo | Y | H | QCH₃ |
| T-228 | H | H | CH₂CH₃ | Oxo | Y | Halo | H |
| T-229 | H | H | CH₂CH₃ | Oxo | Y | QCH₂CH₃ | H |
| T-230 | H | H | CH₂CH₃ | Oxo | Y | QCH₃ | H |
| T-231 | H | H | CH₂CH₃ | H | CH₃ | H | H |
| T-232 | H | H | CH₂CH₃ | H | CH₃ | H | Halo |
| T-233 | H | H | CH₂CH₃ | H | CH₃ | H | QCH₂CH₃ |
| T-234 | H | H | CH₂CH₃ | H | CH₃ | H | QCH₃ |
| T-235 | H | H | CH₂CH₃ | H | CH₃ | Halo | H |
| T-236 | H | H | CH₂CH₃ | H | CH₃ | QCH₂CH₃ | H |
| T-237 | H | H | CH₂CH₃ | H | CH₃ | QCH₃ | H |
| T-238 | H | H | CH₂CH₃ | H | H | H | QCH₂CH₃ |
| T-239 | H | H | CH₂CH₃ | H | H | H | QCH₃ |
| T-240 | H | H | CH₂CH₃ | H | H | QCH₂CH₃ | H |
| T-241 | H | H | CH₂CH₃ | H | H | QCH₃ | H |
| T-242 | H | H | CH₂CH₃ | H | Y | H | H |
| T-243 | H | H | CH₂CH₃ | H | Y | H | QCH₂CH₃ |
| T-244 | H | H | CH₂CH₃ | H | Y | H | QCH₃ |
| T-245 | H | H | CH₂CH₃ | H | Y | QCH₂CH₃ | H |
| T-246 | H | H | CH₂CH₃ | H | Y | QCH₃ | H |
| T-247 | H | H | CH₃ | Oxo | CH₃ | H | Halo |
| T-248 | H | H | CH₃ | Oxo | CH₃ | H | QCH₃ |
| T-249 | H | H | CH₃ | Oxo | CH₃ | Halo | H |
| T-250 | H | H | CH₃ | Oxo | CH₃ | QCH₃ | H |
| T-251 | H | H | CH₃ | Oxo | H | H | Halo |
| T-252 | H | H | CH₃ | Oxo | H | Halo | H |
| T-253 | H | H | CH₃ | Oxo | Y | H | H |
| T-254 | H | H | CH₃ | Oxo | Y | H | Halo |
| T-255 | H | H | CH₃ | Oxo | Y | H | QCH₂CH₃ |
| T-256 | H | H | CH₃ | Oxo | Y | H | QCH₃ |
| T-257 | H | H | CH₃ | Oxo | Y | Halo | H |
| T-258 | H | H | CH₃ | Oxo | Y | QCH₂CH₃ | H |
| T-259 | H | H | CH₃ | Oxo | Y | QCH₃ | H |
| T-260 | H | H | CH₃ | H | CH₃ | H | H |
| T-261 | H | H | CH₃ | H | CH₃ | H | Halo |
| T-262 | H | H | CH₃ | H | CH₃ | H | QCH₂CH₃ |
| T-263 | H | H | CH₃ | H | CH₃ | H | QCH₃ |
| T-264 | H | H | CH₃ | H | CH₃ | Halo | H |
| T-265 | H | H | CH₃ | H | CH₃ | QCH₂CH₃ | H |
| T-266 | H | H | CH₃ | H | CH₃ | QCH₃ | H |
| T-267 | H | H | CH₃ | H | H | H | QCH₂CH₃ |
| T-268 | H | H | CH₃ | H | H | QCH₂CH₃ | H |
| T-269 | H | H | CH₃ | H | Y | H | H |
| T-270 | H | H | CH₃ | H | Y | H | QCH₂CH₃ |
| T-271 | H | H | CH₃ | H | Y | H | QCH₃ |
| T-272 | H | H | CH₃ | H | Y | QCH₂CH₃ | H |
| T-273 | H | H | CH₃ | H | Y | QCH₃ | H |
| T-274 | H | H | H | Oxo | CH₃ | H | Halo |
| T-275 | H | H | H | Oxo | CH₃ | H | QCH₃ |
| T-276 | H | H | H | Oxo | CH₃ | Halo | H |
| T-277 | H | H | H | Oxo | CH₃ | QCH₃ | H |
| T-278 | H | H | H | Oxo | H | H | Halo |
| T-279 | H | H | H | Oxo | H | Halo | H |
| T-280 | H | H | H | Oxo | Y | H | H |
| T-281 | H | H | H | Oxo | Y | H | Halo |
| T-282 | H | H | H | Oxo | Y | H | QCH₂CH₃ |
| T-283 | H | H | H | Oxo | Y | H | QCH₃ |
| T-284 | H | H | H | Oxo | Y | Halo | H |
| T-285 | H | H | H | Oxo | Y | QCH₂CH₃ | H |
| T-286 | H | H | H | Oxo | Y | QCH₃ | H |
| T-287 | H | H | H | H | CH₃ | H | Halo |
| T-288 | H | H | H | H | CH₃ | Halo | H |
| T-289 | H | H | H | H | H | H | QCH₂CH₃ |
| T-290 | H | H | H | H | H | QCH₂CH₃ | H |
| T-291 | H | H | H | H | Y | H | H |
| T-292 | H | H | H | H | Y | H | QCH₂CH₃ |
| T-293 | H | H | H | H | Y | H | QCH₃ |
| T-294 | H | H | H | H | Y | QCH₂CH₃ | H |
| T-295 | H | H | H | H | Y | QCH₃ | H |
| T-296 | Y | CH₂CH₃ | CH₂CH₃ | Oxo | CH₃ | H | H |
| T-297 | Y | CH₂CH₃ | CH₂CH₃ | Oxo | CH₃ | H | Halo |
| T-298 | Y | CH₂CH₃ | CH₂CH₃ | Oxo | CH₃ | H | QCH₂CH₃ |
| T-299 | Y | CH₂CH₃ | CH₂CH₃ | Oxo | CH₃ | H | QCH₃ |
| T-300 | Y | CH₂CH₃ | CH₂CH₃ | Oxo | CH₃ | Halo | H |
| T-301 | Y | CH₂CH₃ | CH₂CH₃ | Oxo | CH₃ | QCH₂CH₃ | H |
| T-302 | Y | CH₂CH₃ | CH₂CH₃ | Oxo | CH₃ | QCH₃ | H |
| T-303 | Y | CH₂CH₃ | CH₂CH₃ | Oxo | H | H | H |
| T-304 | Y | CH₂CH₃ | CH₂CH₃ | Oxo | H | H | Halo |
| T-305 | Y | CH₂CH₃ | CH₂CH₃ | Oxo | H | H | QCH₂CH₃ |
| T-306 | Y | CH₂CH₃ | CH₂CH₃ | Oxo | H | H | QCH₃ |
| T-307 | Y | CH₂CH₃ | CH₂CH₃ | Oxo | H | Halo | H |
| T-308 | Y | CH₂CH₃ | CH₂CH₃ | Oxo | H | QCH₂CH₃ | H |
| T-309 | Y | CH₂CH₃ | CH₂CH₃ | Oxo | H | QCH₃ | H |
| T-310 | Y | CH₂CH₃ | CH₂CH₃ | Oxo | Y | H | H |
| T-311 | Y | CH₂CH₃ | CH₂CH₃ | Oxo | Y | H | Halo |
| T-312 | Y | CH₂CH₃ | CH₂CH₃ | Oxo | Y | H | QCH₂CH₃ |
| T-313 | Y | CH₂CH₃ | CH₂CH₃ | Oxo | Y | H | QCH₃ |
| T-314 | Y | CH₂CH₃ | CH₂CH₃ | Oxo | Y | Halo | H |
| T-315 | Y | CH₂CH₃ | CH₂CH₃ | Oxo | Y | QCH₂CH₃ | H |
| T-316 | Y | CH₂CH₃ | CH₂CH₃ | Oxo | Y | QCH₃ | H |
| T-317 | Y | CH₂CH₃ | CH₂CH₃ | H | CH₃ | H | H |
| T-318 | Y | CH₂CH₃ | CH₂CH₃ | H | CH₃ | H | Halo |
| T-319 | Y | CH₂CH₃ | CH₂CH₃ | H | CH₃ | H | QCH₂CH₃ |
| T-320 | Y | CH₂CH₃ | CH₂CH₃ | H | CH₃ | H | QCH₃ |
| T-321 | Y | CH₂CH₃ | CH₂CH₃ | H | CH₃ | Halo | H |
| T-322 | Y | CH₂CH₃ | CH₂CH₃ | H | CH₃ | QCH₂CH₃ | H |
| T-323 | Y | CH₂CH₃ | CH₂CH₃ | H | CH₃ | QCH₃ | H |
| T-324 | Y | CH₂CH₃ | CH₂CH₃ | H | H | H | H |
| T-325 | Y | CH₂CH₃ | CH₂CH₃ | H | H | H | Halo |
| T-326 | Y | CH₂CH₃ | CH₂CH₃ | H | H | H | QCH₂CH₃ |
| T-327 | Y | CH₂CH₃ | CH₂CH₃ | H | H | H | QCH₃ |
| T-328 | Y | CH₂CH₃ | CH₂CH₃ | H | H | Halo | H |
| T-329 | Y | CH₂CH₃ | CH₂CH₃ | H | H | QCH₂CH₃ | H |
| T-330 | Y | CH₂CH₃ | CH₂CH₃ | H | H | QCH₃ | H |
| T-331 | Y | CH₂CH₃ | CH₂CH₃ | H | Y | H | H |
| T-332 | Y | CH₂CH₃ | CH₂CH₃ | H | Y | H | Halo |
| T-333 | Y | CH₂CH₃ | CH₂CH₃ | H | Y | H | QCH₂CH₃ |
| T-334 | Y | CH₂CH₃ | CH₂CH₃ | H | Y | H | QCH₃ |
| T-335 | Y | CH₂CH₃ | CH₂CH₃ | H | Y | Halo | H |
| T-336 | Y | CH₂CH₃ | CH₂CH₃ | H | Y | QCH₂CH₃ | H |
| T-337 | Y | CH₂CH₃ | CH₂CH₃ | H | Y | QCH₃ | H |
| T-338 | Y | CH₂CH₃ | CH₃ | Oxo | CH₃ | H | H |
| T-339 | Y | CH₂CH₃ | CH₃ | Oxo | CH₃ | H | Halo |
| T-340 | Y | CH₂CH₃ | CH₃ | Oxo | CH₃ | H | QCH₂CH₃ |
| T-341 | Y | CH₂CH₃ | CH₃ | Oxo | CH₃ | H | QCH₃ |
| T-342 | Y | CH₂CH₃ | CH₃ | Oxo | CH₃ | Halo | H |
| T-343 | Y | CH₂CH₃ | CH₃ | Oxo | CH₃ | QCH₂CH₃ | H |
| T-344 | Y | CH₂CH₃ | CH₃ | Oxo | CH₃ | QCH₃ | H |
| T-345 | Y | CH₂CH₃ | CH₃ | Oxo | H | H | H |
| T-346 | Y | CH₂CH₃ | CH₃ | Oxo | H | H | Halo |
| T-347 | Y | CH₂CH₃ | CH₃ | Oxo | H | H | QCH₂CH₃ |
| T-348 | Y | CH₂CH₃ | CH₃ | Oxo | H | H | QCH₃ |
| T-349 | Y | CH₂CH₃ | CH₃ | Oxo | H | Halo | H |
| T-350 | Y | CH₂CH₃ | CH₃ | Oxo | H | QCH₂CH₃ | H |
| T-351 | Y | CH₂CH₃ | CH₃ | Oxo | H | QCH₃ | H |
| T-352 | Y | CH₂CH₃ | CH₃ | Oxo | Y | H | H |
| T-353 | Y | CH₂CH₃ | CH₃ | Oxo | Y | H | Halo |
| T-354 | Y | CH₂CH₃ | CH₃ | Oxo | Y | H | QCH₂CH₃ |
| T-355 | Y | CH₂CH₃ | CH₃ | Oxo | Y | H | QCH₃ |
| T-356 | Y | CH₂CH₃ | CH₃ | Oxo | Y | Halo | H |
| T-357 | Y | CH₂CH₃ | CH₃ | Oxo | Y | QCH₂CH₃ | H |
| T-358 | Y | CH₂CH₃ | CH₃ | Oxo | Y | QCH₃ | H |
| T-359 | Y | CH₂CH₃ | CH₃ | H | CH₃ | H | H |
| T-360 | Y | CH₂CH₃ | CH₃ | H | CH₃ | H | Halo |
| T-361 | Y | CH₂CH₃ | CH₃ | H | CH₃ | H | QCH₂CH₃ |
| T-362 | Y | CH₂CH₃ | CH₃ | H | CH₃ | H | QCH₃ |
| T-363 | Y | CH₂CH₃ | CH₃ | H | CH₃ | Halo | H |
| T-364 | Y | CH₂CH₃ | CH₃ | H | CH₃ | QCH₂CH₃ | H |
| T-365 | Y | CH₂CH₃ | CH₃ | H | CH₃ | QCH₃ | H |
| T-366 | Y | CH₂CH₃ | CH₃ | H | H | H | H |
| T-367 | Y | CH₂CH₃ | CH₃ | H | H | H | Halo |
| T-368 | Y | CH₂CH₃ | CH₃ | H | H | H | QCH₂CH₃ |
| T-369 | Y | CH₂CH₃ | CH₃ | H | H | H | QCH₃ |
| T-370 | Y | CH₂CH₃ | CH₃ | H | H | Halo | H |
| T-371 | Y | CH₂CH₃ | CH₃ | H | H | QCH₂CH₃ | H |
| T-372 | Y | CH₂CH₃ | CH₃ | H | H | QCH₃ | H |
| T-373 | Y | CH₂CH₃ | CH₃ | H | Y | H | H |
| T-374 | Y | CH₂CH₃ | CH₃ | H | Y | H | Halo |
| T-375 | Y | CH₂CH₃ | CH | H | Y | H | QCH₂CH₃ |
| T-376 | Y | CH₂CH₃ | CH₃ | H | Y | H | QCH₃ |
| T-377 | Y | CH₂CH₃ | CH₃ | H | Y | Halo | H |
| T-378 | Y | CH₂CH₃ | CH₃ | H | Y | QCH₂CH₃ | H |
| T-379 | Y | CH₂CH₃ | CH₃ | H | Y | QCH₃ | H |
| T-380 | Y | CH₂CH₃ | H | Oxo | CH₃ | H | H |
| T-381 | Y | CH₂CH₃ | H | Oxo | CH₃ | H | Halo |
| T-382 | Y | CH₂CH₃ | H | Oxo | CH₃ | H | QCH₂CH₃ |
| T-383 | Y | CH₂CH₃ | H | Oxo | CH₃ | H | QCH₃ |
| T-384 | Y | CH₂CH₃ | H | Oxo | CH₃ | Halo | H |
| T-385 | Y | CH₂CH₃ | H | Oxo | CH₃ | QCH₂CH₃ | H |
| T-386 | Y | CH₂CH₃ | H | Oxo | CH₃ | QCH₃ | H |
| T-387 | Y | CH₂CH₃ | H | Oxo | H | H | H |
| T-388 | Y | CH₂CH₃ | H | Oxo | H | H | Halo |
| T-389 | Y | CH₂CH₃ | H | Oxo | H | H | QCH₂CH₃ |
| T-390 | Y | CH₂CH₃ | H | Oxo | H | H | QCH₃ |
| T-391 | Y | CH₂CH₃ | H | Oxo | H | Halo | H |
| T-392 | Y | CH₂CH₃ | H | Oxo | H | QCH₂CH₃ | H |
| T-393 | Y | CH₂CH₃ | H | Oxo | H | QCH₃ | H |
| T-394 | Y | CH₂CH₃ | H | Oxo | Y | H | H |
| T-395 | Y | CH₂CH₃ | H | Oxo | Y | H | Halo |
| T-396 | Y | CH₂CH₃ | H | Oxo | Y | H | QCH₂CH₃ |
| T-397 | Y | CH₂CH₃ | H | Oxo | Y | H | QCH₃ |
| T-398 | Y | CH₂CH₃ | H | Oxo | Y | Halo | H |
| T-399 | Y | CH₂CH₃ | H | Oxo | Y | QCH₂CH₃ | H |
| T-400 | Y | CH₂CH₃ | H | Oxo | Y | QCH₃ | H |
| T-401 | Y | CH₂CH₃ | H | H | CH₃ | H | H |
| T-402 | Y | CH₂CH₃ | H | H | CH₃ | H | Halo |
| T-403 | Y | CH₂CH₃ | H | H | CH₃ | H | QCH₂CH₃ |
| T-404 | Y | CH₂CH₃ | H | H | CH₃ | H | QCH₃ |
| T-405 | Y | CH₂CH₃ | H | H | CH₃ | Halo | H |
| T-406 | Y | CH₂CH₃ | H | H | CH₃ | QCH₂CH₃ | H |
| T-407 | Y | CH₂CH₃ | H | H | CH₃ | QCH₃ | H |
| T-408 | Y | CH₂CH₃ | H | H | H | H | H |
| T-409 | Y | CH₂CH₃ | H | H | H | H | QCH₂CH₃ |
| T-410 | Y | CH₂CH₃ | H | H | H | H | QCH₃ |
| T-411 | Y | CH₂CH₃ | H | H | H | QCH₂CH₃ | H |
| T-412 | Y | CH₂CH₃ | H | H | H | QCH₃ | H |
| T-413 | Y | CH₂CH₃ | H | H | Y | H | H |
| T-414 | Y | CH₂CH₃ | H | H | Y | H | QCH₂CH₃ |
| T-415 | Y | CH₂CH₃ | H | H | Y | H | QCH₃ |
| T-416 | Y | CH₂CH₃ | H | H | Y | QCH₂CH₃ | H |
| T-417 | Y | CH₂CH₃ | H | H | Y | QCH₃ | H |
| T-418 | Y | CH₃ | CH₂CH₃ | Oxo | CH₃ | H | H |
| T-419 | Y | CH₃ | CH₂CH₃ | Oxo | CH₃ | H | Halo |
| T-420 | Y | CH₃ | CH₂CH₃ | Oxo | CH₃ | H | QCH₂CH₃ |
| T-421 | Y | CH₃ | CH₂CH₃ | Oxo | CH₃ | H | QCH₃ |
| T-422 | Y | CH₃ | CH₂CH₃ | Oxo | CH₃ | Halo | H |
| T-423 | Y | CH₃ | CH₂CH₃ | Oxo | CH₃ | QCH₂CH₃ | H |
| T-424 | Y | CH₃ | CH₂CH₃ | Oxo | CH₃ | QCH₃ | H |
| T-425 | Y | CH | CH₂CH₃ | Oxo | H | H | H |
| T-426 | Y | CH₃ | CH₂CH₃ | Oxo | H | H | Halo |
| T-427 | Y | CH₃ | CH₂CH₃ | Oxo | H | H | QCH₂CH₃ |
| T-428 | Y | CH₃ | CH₂CH₃ | Oxo | H | H | QCH₃ |
| T-429 | Y | CH₃ | CH₂CH₃ | Oxo | H | Halo | H |
| T-430 | Y | CH₃ | CH₂CH₃ | Oxo | H | QCH₂CH₃ | H |
| T-431 | Y | CH₃ | CH₂CH₃ | Oxo | H | QCH₃ | H |
| T-432 | Y | CH₃ | CH₂CH₃ | Oxo | Y | H | H |
| T-433 | Y | CH₃ | CH₂CH₃ | Oxo | Y | H | Halo |
| T-434 | Y | CH₃ | CH₂CH₃ | Oxo | Y | H | QCH₂CH₃ |
| T-435 | Y | CH₃ | CH₂CH₃ | Oxo | Y | H | QCH₃ |
| T-436 | Y | CH₃ | CH₂CH₃ | Oxo | Y | Halo | H |
| T-437 | Y | CH₃ | CH₂CH₃ | Oxo | Y | QCH₂CH₃ | H |
| T-438 | Y | CH₃ | CH₂CH₃ | Oxo | Y | QCH₃ | H |
| T-439 | Y | CH₃ | CH₂CH₃ | H | CH₃ | H | H |
| T-440 | Y | CH₃ | CH₂CH₃ | H | CH₃ | H | Halo |
| T-441 | Y | CH₃ | CH₂CH₃ | H | CH₃ | H | QCH₂CH₃ |
| T-442 | Y | CH₃ | CH₂CH₃ | H | CH₃ | H | QCH₃ |
| T-443 | Y | CH₃ | CH₂CH₃ | H | CH₃ | Halo | H |
| T-444 | Y | CH₃ | CH₂CH₃ | H | CH₃ | QCH₂CH₃ | H |
| T-445 | Y | CH₃ | CH₂CH₃ | H | CH₃ | QCH₃ | H |
| T-446 | Y | CH₃ | CH₂CH₃ | H | H | H | H |
| T-447 | Y | CH₃ | CH₂CH₃ | H | H | H | Halo |
| T-448 | Y | CH₃ | CH₂CH₃ | H | H | H | QCH₂CH₃ |
| T-449 | Y | CH₃ | CH₂CH₃ | H | H | H | QCH₃ |
| T-450 | Y | CH₃ | CH₂CH₃ | H | H | Halo | H |
| T-451 | Y | CH₃ | CH₂CH₃ | H | H | QCH₂CH₃ | H |
| T-452 | Y | CH₃ | CH₂CH₃ | H | H | QCH₃ | H |
| T-453 | Y | CH₃ | CH₂CH₃ | H | Y | H | H |
| T-454 | Y | CH₃ | CH₂CH₃ | H | Y | H | Halo |
| T-455 | Y | CH₃ | CH₂CH₃ | H | Y | H | QCH₂CH₃ |
| T-456 | Y | CH₃ | CH₂CH₃ | H | Y | H | QCH₃ |
| T-457 | Y | CH₃ | CH₂CH₃ | H | Y | Halo | H |
| T-458 | Y | CH₃ | CH₂CH₃ | H | Y | QCH₂CH₃ | H |
| T-459 | Y | CH₃ | CH₂CH₃ | H | Y | QCH₃ | H |
| T-460 | Y | CH₃ | CH₃ | Oxo | CH₃ | H | H |
| T-461 | Y | CH₃ | CH₃ | Oxo | CH₃ | H | Halo |
| T-462 | Y | CH₃ | CH₃ | Oxo | CH₃ | H | QCH₂CH₃ |
| T-463 | Y | CH₃ | CH₃ | Oxo | CH₃ | H | QCH₃ |
| T-464 | Y | CH₃ | CH₃ | Oxo | CH₃ | Halo | H |
| T-465 | Y | CH₃ | CH₃ | Oxo | CH₃ | QCH₂CH₃ | H |
| T-466 | Y | CH₃ | CH₃ | Oxo | CH₃ | QCH₃ | H |
| T-467 | Y | CH₃ | CH₃ | Oxo | H | H | H |
| T-468 | Y | CH₃ | CH₃ | Oxo | H | H | Halo |
| T-469 | Y | CH₃ | CH₃ | Oxo | H | H | QCH₂CH₃ |
| T-470 | Y | CH₃ | CH₃ | Oxo | H | H | QCH₃ |
| T-471 | Y | CH₃ | CH₃ | Oxo | H | Halo | H |
| T-472 | Y | CH₃ | CH₃ | Oxo | H | QCH₂CH₃ | H |
| T-473 | Y | CH₃ | CH₃ | Oxo | H | QCH₃ | H |
| T-474 | Y | CH₃ | CH₃ | Oxo | Y | H | H |
| T-475 | Y | CH₃ | CH₃ | Oxo | Y | H | Halo |
| T-476 | Y | CH₃ | CH₃ | Oxo | Y | H | QCH₂CH₃ |
| T-477 | Y | CH₃ | CH₃ | Oxo | Y | H | QCH₃ |
| T-478 | Y | CH₃ | CH₃ | Oxo | Y | Halo | H |
| T-479 | Y | CH₃ | CH₃ | Oxo | Y | QCH₂CH₃ | H |
| T-480 | Y | CH₃ | CH₃ | Oxo | Y | QCH₃ | H |
| T-481 | Y | CH₃ | CH₃ | H | CH₃ | H | H |
| T-482 | Y | CH₃ | CH₃ | H | CH₃ | H | Halo |
| T-483 | Y | CH₃ | CH₃ | H | CH₃ | H | QCH₂CH₃ |
| T-484 | Y | CH₃ | CH₃ | H | CH₃ | H | QCH₃ |
| T-485 | Y | CH₃ | CH₃ | H | CH₃ | Halo | H |
| T-486 | Y | CH₃ | CH₃ | H | CH₃ | QCH₂CH₃ | H |
| T-487 | Y | CH₃ | CH₃ | H | CH₃ | QCH₃ | H |
| T-488 | Y | CH₃ | CH₃ | H | H | H | H |
| T-489 | Y | CH₃ | CH₃ | H | H | H | Halo |
| T-490 | Y | CH₃ | CH₃ | H | H | H | QCH₂CH₃ |
| T-491 | Y | CH₃ | CH₃ | H | H | H | QCH₃ |
| T-492 | Y | CH₃ | CH₃ | H | H | Halo | H |
| T-493 | Y | CH₃ | CH₃ | H | H | QCH₂CH₃ | H |
| T-494 | Y | CH₃ | CH₃ | H | H | QCH₃ | H |
| T-495 | Y | CH₃ | CH₃ | H | Y | H | H |
| T-496 | Y | CH₃ | CH₃ | H | Y | H | Halo |
| T-497 | Y | CH₃ | CH₃ | H | Y | H | QCH₂CH₃ |
| T-498 | Y | CH₃ | CH₃ | H | Y | H | QCH₃ |
| T-499 | Y | CH₃ | CH₃ | H | Y | Halo | H |
| T-500 | Y | CH₃ | CH₃ | H | Y | QCH₂CH₃ | H |
| T-501 | Y | CH₃ | CH₃ | H | Y | QCH₃ | H |
| T-502 | Y | CH₃ | H | Oxo | CH₃ | H | H |
| T-503 | Y | CH₃ | H | Oxo | CH₃ | H | Halo |
| T-504 | Y | CH₃ | H | Oxo | CH₃ | H | QCH₂CH₃ |
| T-505 | Y | CH₃ | H | Oxo | CH₃ | H | QCH₃ |
| T-506 | Y | CH₃ | H | Oxo | CH₃ | Halo | H |
| T-507 | Y | CH₃ | H | Oxo | CH₃ | QCH₂CH₃ | H |
| T-508 | Y | CH₃ | H | Oxo | CH₃ | QCH₃ | H |
| T-509 | Y | CH₃ | H | Oxo | H | H | H |
| T-510 | Y | CH₃ | H | Oxo | H | H | Halo |
| T-511 | Y | CH₃ | H | Oxo | H | H | QCH₂CH₃ |
| T-512 | Y | CH₃ | H | Oxo | H | H | QCH₃ |
| T-513 | Y | CH₃ | H | Ox | H | Halo | H |
| T-514 | Y | CH₃ | H | Oxo | H | QCH₂CH₃ | H |
| T-515 | Y | CH₃ | H | Oxo | H | QCH₃ | H |
| T-516 | Y | CH₃ | H | Oxo | Y | H | H |
| T-517 | Y | CH₃ | H | Oxo | Y | H | Halo |
| T-518 | Y | CH₃ | H | Oxo | Y | H | QCH₂CH₃ |
| T-519 | Y | CH₃ | H | Oxo | Y | H | QCH₃ |
| T-520 | Y | CH₃ | H | Oxo | Y | Halo | H |
| T-521 | Y | CH₃ | H | Oxo | Y | QCH₂CH₃ | H |
| T-522 | Y | CH₃ | H | Oxo | Y | QCH₃ | H |
| T-523 | Y | CH₃ | H | H | CH₃ | H | H |
| T-524 | Y | CH₃ | H | H | CH₃ | H | Halo |
| T-525 | Y | CH₃ | H | H | CH₃ | H | QCH₂CH₃ |
| T-526 | Y | CH₃ | H | H | CH₃ | H | QCH₃ |
| T-527 | Y | CH₃ | H | H | CH₃ | Halo | H |
| T-528 | Y | CH₃ | H | H | CH₃ | QCH₂CH₃ | H |
| T-529 | Y | CH₃ | H | H | CH₃ | QCH₃ | H |
| T-530 | Y | CH₃ | H | H | H | H | H |
| T-531 | Y | CH₃ | H | H | H | H | QCH₂CH₃ |
| T-532 | Y | CH₃ | H | H | H | H | QCH₃ |
| T-533 | Y | CH₃ | H | H | H | QCH₂CH₃ | H |
| T-534 | Y | CH₃ | H | H | H | QCH₃ | H |
| T-535 | Y | CH₃ | H | H | Y | H | H |
| T-536 | Y | CH₃ | H | H | Y | H | QCH₂CH₃ |
| T-537 | Y | CH₃ | H | H | Y | H | QCH₃ |
| T-538 | Y | CH₃ | H | H | Y | QCH₂CH₃ | H |
| T-539 | Y | CH₃ | H | H | Y | QCH₃ | H |
| T-540 | Y | H | CH₂CH₃ | Oxo | CH₃ | H | H |
| T-541 | Y | H | CH₂CH₃ | Oxo | CH₃ | H | Halo |
| T-542 | Y | H | CH₂CH₃ | Oxo | CH₃ | H | QCH₂CH₃ |
| T-543 | Y | H | CH₂CH₃ | Oxo | CH₃ | H | QCH₃ |
| T-544 | Y | H | CH₂CH₃ | Oxo | CH₃ | Halo | H |
| T-545 | Y | H | CH₂CH₃ | Oxo | CH₃ | QCH₂CH₃ | H |
| T-546 | Y | H | CH₂CH₃ | Oxo | CH₃ | QCH₃ | H |
| T-547 | Y | H | CH₂CH₃ | Oxo | H | H | H |
| T-548 | Y | H | CH₂CH₃ | Oxo | H | H | Halo |
| T-549 | Y | H | CH₂CH₃ | Oxo | H | H | QCH₂CH₃ |
| T-550 | Y | H | CH₂CH₃ | Oxo | H | H | QCH₃ |
| T-551 | Y | H | CH₂CH₃ | Oxo | H | Halo | H |
| T-552 | Y | H | CH₂CH₃ | Oxo | H | QCH₂CH₃ | H |
| T-553 | Y | H | CH₂CH₃ | Oxo | H | QCH₃ | H |
| T-554 | Y | H | CH₂CH₃ | Oxo | Y | H | H |
| T-555 | Y | H | CH₂CH₃ | Oxo | Y | H | Halo |
| T-556 | Y | H | CH₂CH₃ | Oxo | Y | H | QCH₂CH₃ |
| T-557 | Y | H | CH₂CH₃ | Oxo | Y | H | QCH₃ |
| T-558 | Y | H | CH₂CH₃ | Oxo | Y | Halo | H |
| T-559 | Y | H | CH₂CH₃ | Oxo | Y | QCH₂CH₃ | H |
| T-560 | Y | H | CH₂CH₃ | Oxo | Y | QCH₃ | H |
| T-561 | Y | H | CH₂CH₃ | H | CH₃ | H | H |
| T-562 | Y | H | CH₂CH₃ | H | CH | H | Halo |
| T-563 | Y | H | CH₂CH₃ | H | CH₃ | H | QCH₂CH₃ |
| T-564 | Y | H | CH₂CH₃ | H | CH₃ | H | QCH₃ |
| T-565 | Y | H | CH₂CH₃ | H | CH₃ | Halo | H |
| T-566 | Y | H | CH₂CH₃ | H | CH₃ | QCH₂CH₃ | H |
| T-567 | Y | H | CH₂CH₃ | H | CH₃ | QCH₃ | H |
| T-568 | Y | H | CH₂CH₃ | H | H | H | H |
| T-569 | Y | H | CH₂CH₃ | H | H | H | QCH₂CH₃ |
| T-570 | Y | H | CH₂CH₃ | H | H | H | QCH₃ |
| T-571 | Y | H | CH₂CH₃ | H | H | QCH₂CH₃ | H |
| T-572 | Y | H | CH₂CH₃ | H | H | QCH₃ | H |
| T-573 | Y | H | CH₂CH₃ | H | Y | H | H |
| T-574 | Y | H | CH₂CH₃ | H | Y | H | QCH₂CH₃ |
| T-575 | Y | H | CH₂CH₃ | H | Y | H | QCH₃ |
| T-576 | Y | H | CH₂CH₃ | H | Y | QCH₂CH₃ | H |
| T-577 | Y | H | CH₂CH₃ | H | Y | QCH₃ | H |
| T-578 | Y | H | CH₃ | Oxo | CH₃ | H | H |
| T-579 | Y | H | CH₃ | Oxo | CH₃ | H | Halo |
| T-580 | Y | H | CH₃ | Oxo | CH₃ | H | QCH₂CH₃ |
| T-581 | Y | H | CH₃ | Oxo | CH₃ | H | QCH₃ |
| T-582 | Y | H | CH₃ | Oxo | CH₃ | Halo | H |
| T-583 | Y | H | CH₃ | Oxo | CH₃ | QCH₂CH₃ | H |
| T-584 | Y | H | CH₃ | Oxo | CH₃ | QCH₃ | H |
| T-585 | Y | H | CH₃ | Oxo | H | H | H |
| T-586 | Y | H | CH₃ | Oxo | H | H | Halo |
| T-587 | Y | H | CH₃ | Oxo | H | H | QCH₂CH₃ |
| T-588 | Y | H | CH₃ | Oxo | H | H | QCH₃ |
| T-589 | Y | H | CH₃ | Oxo | H | Halo | H |
| T-590 | Y | H | CH₃ | Oxo | H | QCH₂CH₃ | H |
| T-591 | Y | H | CH₃ | Oxo | H | QCH₃ | H |
| T-592 | Y | H | CH₃ | Oxo | Y | H | H |
| T-593 | Y | H | CH₃ | Oxo | Y | H | Halo |
| T-594 | Y | H | CH₃ | Oxo | Y | H | QCH₂CH₃ |
| T-595 | Y | H | CH₃ | Oxo | Y | H | QCH₃ |
| T-596 | Y | H | CH₃ | Oxo | Y | Halo | H |
| T-597 | Y | H | CH₃ | Oxo | Y | QCH₂CH₃ | H |
| T-598 | Y | H | CH₃ | Oxo | Y | QCH₃ | H |
| T-599 | Y | H | CH₃ | H | CH₃ | H | H |
| T-600 | Y | H | CH₃ | H | CH₃ | H | Halo |
| T-601 | Y | H | CH₃ | H | CH₃ | H | QCH₂CH₃ |
| T-602 | Y | H | CH₃ | H | CH₃ | H | QCH₃ |
| T-603 | Y | H | CH₃ | H | CH₃ | Halo | H |
| T-604 | Y | H | CH₃ | H | CH₃ | QCH₂CH₃ | H |
| T-605 | Y | H | CH₃ | H | CH₃ | QCH₃ | H |
| T-606 | Y | H | CH₃ | H | H | H | H |
| T-607 | Y | H | CH₃ | H | H | H | QCH₂CH₃ |
| T-608 | Y | H | CH₃ | H | H | H | QCH₃ |
| T-609 | Y | H | CH₃ | H | H | QCH₂CH₃ | H |
| T-610 | Y | H | CH₃ | H | H | QCH₃ | H |
| T-611 | Y | H | CH₃ | H | Y | H | H |
| T-612 | Y | H | CH₃ | H | Y | H | QCH₂CH₃ |
| T-613 | Y | H | CH₃ | H | Y | H | QCH₃ |
| T-614 | Y | H | CH₃ | H | Y | QCH₂CH₃ | H |
| T-615 | Y | H | CH₃ | H | Y | QCH₃ | H |
| T-616 | Y | H | H | Oxo | CH₃ | H | H |
| T-617 | Y | H | H | Oxo | CH₃ | H | Halo |
| T-618 | Y | H | H | Oxo | CH₃ | H | QCH₂CH₃ |
| T-619 | Y | H | H | Oxo | CH₃ | H | QCH₃ |
| T-620 | Y | H | H | Oxo | CH₃ | Halo | H |
| T-621 | Y | H | H | Oxo | CH₃ | QCH₂CH₃ | H |
| T-622 | Y | H | H | Oxo | CH₃ | QCH₃ | H |
| T-623 | Y | H | H | Oxo | H | H | H |
| T-624 | Y | H | H | Oxo | H | H | Halo |
| T-625 | Y | H | H | Oxo | H | H | QCH₂CH₃ |
| T-626 | Y | H | H | Oxo | H | H | QCH₃ |
| T-627 | Y | H | H | Oxo | H | Halo | H |
| T-628 | Y | H | H | Oxo | H | QCH₂CH₃ | H |
| T-629 | Y | H | H | Oxo | H | QCH₃ | H |
| T-630 | Y | H | H | Oxo | Y | H | H |
| T-631 | Y | H | H | Oxo | Y | H | Halo |
| T-632 | Y | H | H | Oxo | Y | H | QCH₂CH₃ |
| T-633 | Y | H | H | Oxo | Y | H | QCH₃ |
| T-634 | Y | H | H | Oxo | Y | Halo | H |
| T-635 | Y | H | H | Oxo | Y | QCH₂CH₃ | H |
| T-636 | Y | H | H | Oxo | Y | QCH₃ | H |
| T-637 | Y | H | H | H | CH₃ | H | H |
| T-638 | Y | H | H | H | CH₃ | H | Halo |
| T-639 | Y | H | H | H | CH₃ | H | QCH₂CH₃ |
| T-640 | Y | H | H | H | CH₃ | H | QCH₃ |
| T-641 | Y | H | H | H | CH₃ | Halo | H |
| T-642 | Y | H | H | H | CH₃ | QCH₂CH₃ | H |
| T-643 | Y | H | H | H | CH₃ | QCH₃ | H |
| T-644 | Y | H | H | H | H | H | H |
| T-645 | Y | H | H | H | H | H | QCH₂CH₃ |
| T-646 | Y | H | H | H | H | H | QCH₃ |
| T-647 | Y | H | H | H | H | QCH₂CH₃ | H |
| T-648 | Y | H | H | H | H | QCH₃ | H |
| T-649 | Y | H | H | H | Y | H | H |
| T-650 | Y | H | H | H | Y | H | QCH₂CH₃ |
| T-651 | Y | H | H | H | Y | H | QCH₃ |
| T-652 | Y | H | H | H | Y | QCH₂CH₃ | H |
| T-653 | Y | H | H | H | Y | QCH₃ | H |
| T-654 | Y | Y | CH₂CH₃ | Oxo | CH₃ | H | H |
| T-655 | Y | Y | CH₂CH₃ | Oxo | CH₃ | H | Halo |
| T-656 | Y | Y | CH₂CH₃ | Oxo | CH₃ | H | QCH₂CH₃ |
| T-657 | Y | Y | CH₂CH₃ | Oxo | CH₃ | H | QCH₃ |
| T-658 | Y | Y | CH₂CH₃ | Oxo | CH₃ | Halo | H |
| T-659 | Y | Y | CH₂CH₃ | Oxo | CH₃ | QCH₂CH₃ | H |
| T-660 | Y | Y | CH₂CH₃ | Oxo | CH₃ | QCH₃ | H |
| T-661 | Y | Y | CH₂CH₃ | Oxo | H | H | H |
| T-662 | Y | Y | CH₂CH₃ | Oxo | H | H | Halo |
| T-663 | Y | Y | CH₂CH₃ | Oxo | H | H | QCH₂CH₃ |
| T-664 | Y | Y | CH₂CH₃ | Oxo | H | H | QCH₃ |
| T-665 | Y | Y | CH₂CH₃ | Oxo | H | Halo | H |
| T-666 | Y | Y | CH₂CH₃ | Oxo | H | QCH₂CH₃ | H |
| T-667 | Y | Y | CH₂CH₃ | Oxo | H | QCH₃ | H |
| T-668 | Y | Y | CH₂CH₃ | Oxo | Y | H | H |
| T-669 | Y | Y | CH₂CH₃ | Oxo | Y | H | Halo |
| T-670 | Y | Y | CH₂CH₃ | Oxo | Y | H | QCH₂CH₃ |
| T-671 | Y | Y | CH₂CH₃ | Oxo | Y | H | QCH₃ |
| T-672 | Y | Y | CH₂CH₃ | Oxo | Y | Halo | H |
| T-673 | Y | Y | CH₂CH₃ | Oxo | Y | QCH₂CH₃ | H |
| T-674 | Y | Y | CH₂CH₃ | Oxo | Y | QCH₃ | H |
| T-675 | Y | Y | CH₂CH₃ | H | CH₃ | H | H |
| T-676 | Y | Y | CH₂CH₃ | H | CH₃ | H | Halo |
| T-677 | Y | Y | CH₂CH₃ | H | CH₃ | H | QCH₂CH₃ |
| T-678 | Y | Y | CH₂CH₃ | H | CH₃ | H | QCH₃ |
| T-679 | Y | Y | CH₂CH₃ | H | CH₃ | Halo | H |
| T-680 | Y | Y | CH₂CH₃ | H | CH₃ | QCH₂CH₃ | H |
| T-681 | Y | Y | CH₂CH₃ | H | CH₃ | QCH₃ | H |
| T-682 | Y | Y | CH₂CH₃ | H | H | H | H |
| T-683 | Y | Y | CH₂CH₃ | H | H | H | QCH₂CH₃ |
| T-684 | Y | Y | CH₂CH₃ | H | H | H | QCH₃ |
| T-685 | Y | Y | CH₂CH₃ | H | H | QCH₂CH₃ | H |
| T-686 | Y | Y | CH₂CH₃ | H | H | QCH₃ | H |
| T-687 | Y | Y | CH₂CH₃ | H | Y | H | H |
| T-688 | Y | Y | CH₂CH₃ | H | Y | H | QCH₂CH₃ |
| T-689 | Y | Y | CH₂CH₃ | H | Y | H | QCH₃ |
| T-690 | Y | Y | CH₂CH₃ | H | Y | QCH₂CH₃ | H |
| T-691 | Y | Y | CH₂CH₃ | H | Y | QCH₃ | H |
| T-692 | Y | Y | CH₃ | Oxo | CH | H | H |
| T-693 | Y | Y | CH₃ | Oxo | CH₃ | H | Halo |
| T-694 | Y | Y | CH₃ | Oxo | CH₃ | H | QCH₂CH₃ |
| T-695 | Y | Y | CH₃ | Oxo | CH₃ | H | QCH₃ |
| T-696 | Y | Y | CH₃ | Oxo | CH₃ | Halo | H |
| T-697 | Y | Y | CH₃ | Oxo | CH₃ | QCH₂CH₃ | H |
| T-698 | Y | Y | CH₃ | Oxo | CH₃ | QCH₃ | H |
| T-699 | Y | Y | CH₃ | Oxo | H | H | H |
| T-700 | Y | Y | CH₃ | Oxo | H | H | Halo |
| T-701 | Y | Y | CH₃ | Oxo | H | H | QCH₂CH₃ |
| T-702 | Y | Y | CH₃ | Oxo | H | H | QCH₃ |
| T-703 | Y | Y | CH₃ | Oxo | H | Halo | H |
| T-704 | Y | Y | CH₃ | Oxo | H | QCH₂CH₃ | H |
| T-705 | Y | Y | CH₃ | Oxo | H | QCH₃ | H |
| T-706 | Y | Y | CH₃ | Oxo | Y | H | H |
| T-707 | Y | Y | CH₃ | Oxo | Y | H | Halo |
| T-708 | Y | Y | CH₃ | Oxo | Y | H | OCH₂CH₃ |
| T-709 | Y | Y | CH₃ | Oxo | Y | H | OCH₃ |
| T-710 | Y | Y | CH₃ | Oxo | Y | Halo | H |
| T-711 | Y | Y | CH₃ | Oxo | Y | QCH₂CH₃ | H |
| T-712 | Y | Y | CH₃ | Oxo | Y | QCH₃ | H |
| T-713 | Y | Y | CH₃ | H | CH₃ | H | H |
| T-714 | Y | Y | CH₃ | H | CH₃ | H | Halo |
| T-715 | Y | Y | CH₃ | H | CH₃ | H | QCH₂CH₃ |
| T-716 | Y | Y | CH₃ | H | CH₃ | H | QCH₃ |
| T-717 | Y | Y | CH₃ | H | CH₃ | Halo | H |
| T-718 | Y | Y | CH₃ | H | CH₃ | QCH₂CH₃ | H |
| T-719 | Y | Y | CH₃ | H | CH₃ | QCH₃ | H |
| T-720 | Y | Y | CH₃ | H | H | H | H |
| T-721 | Y | Y | CH₃ | H | H | H | QCH₂CH₃ |
| T-722 | Y | Y | CH₃ | H | H | H | QCH₃ |
| T-723 | Y | Y | CH₃ | H | H | QCH₂CH₃ | H |
| T-724 | Y | Y | CH₃ | H | H | QCH₃ | H |
| T-725 | Y | Y | CH₃ | H | Y | H | H |
| T-726 | Y | Y | CH₃ | H | Y | H | QCH₂CH₃ |
| T-727 | Y | Y | CH₃ | H | Y | H | QCH₃ |
| T-728 | Y | Y | CH₃ | H | Y | QCH₂CH₃ | H |
| T-729 | Y | Y | CH₃ | H | Y | QCH₃ | H |
| T-730 | Y | Y | H | Oxo | CH₃ | H | H |
| T-731 | Y | Y | H | Oxo | CH₃ | H | Halo |
| T-732 | Y | Y | H | Oxo | CH₃ | H | QCH₂CH₃ |
| T-733 | Y | Y | H | Oxo | CH₃ | H | QCH₃ |
| T-734 | Y | Y | H | Oxo | CH₃ | Halo | H |
| T-735 | Y | Y | H | Oxo | CH₃ | QCH₂CH₃ | H |
| T-736 | Y | Y | H | Oxo | CH₃ | QCH₃ | H |
| T-737 | Y | Y | H | Oxo | H | H | H |
| T-738 | Y | Y | H | Oxo | H | H | Halo |
| T-739 | Y | Y | H | Oxo | H | H | QCH₂CH₃ |
| T-740 | Y | Y | H | Oxo | H | H | QCH₃ |
| T-741 | Y | Y | H | Oxo | H | Halo | H |
| T-742 | Y | Y | H | Oxo | H | QCH₂CH₃ | H |
| T-743 | Y | Y | H | Oxo | H | QCH₃ | H |
| T-744 | Y | Y | H | Oxo | Y | H | H |
| T-745 | Y | Y | H | Oxo | Y | H | Halo |
| T-746 | Y | Y | H | Oxo | Y | H | QCH₂CH₃ |
| T-747 | Y | Y | H | Oxo | Y | H | QCH₃ |
| T-748 | Y | Y | H | Oxo | Y | Halo | H |
| T-749 | Y | Y | H | Oxo | Y | QCH₂CH₃ | H |
| T-750 | Y | Y | H | Oxo | Y | QCH₃ | H |
| T-751 | Y | Y | H | H | CH₃ | H | H |
| T-752 | Y | Y | H | H | CH₃ | H | Halo |
| T-753 | Y | Y | H | H | CH₃ | H | QCH₂CH₃ |
| T-754 | Y | Y | H | H | CH₃ | H | QCH₃ |
| T-755 | Y | Y | H | H | CH₃ | Halo | H |
| T-756 | Y | Y | H | H | CH₃ | QCH₂CH₃ | H |
| T-757 | Y | Y | H | H | CH₃ | QCH₃ | H |
| T-758 | Y | Y | H | H | H | H | H |
| T-759 | Y | Y | H | H | H | H | QCH₂CH₃ |
| T-760 | Y | Y | H | H | H | H | QCH₃ |
| T-761 | Y | Y | H | H | H | QCH₂CH₃ | H |
| T-762 | Y | Y | H | H | H | QCH₃ | H |
| T-763 | Y | Y | H | H | Y | H | H |
| T-764 | Y | Y | H | H | Y | H | QCH₂CH₃ |
| T-765 | Y | Y | H | H | Y | H | QCH₃ |
| T-766 | Y | Y | H | H | Y | QCH₂CH₃ | H |
| T-767 | Y | Y | H | H | Y | QCH₃ | H |

Certain compounds for use in the invention may also exist in several tautomeric forms including the enol form, the keto form, and mixtures thereof. Accordingly, the chemical structures depicted herein encompass all possible tautomeric forms of the illustrated compounds. Keto-enol tautomerism, for example, is the reversible transfer of a hydrogen from the alpha carbon adjacent to a carbonyl group followed by a double bond transfer. In solution, compounds will spontaneously undergo a kinetic transformation from one tautomer to the other until equilibrium is reached, generally strongly favoring the keto tautomer over the enol tautomer, but dependent on factors such as solvent, pH, and temperature. Keto and enol tautomers may have distinguishable physicochemical properties; however, because they will interconvert in solution, reference to a compound in its keto form (e.g., where Q is

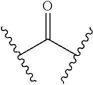 [SMILES: *C(*)=O]

) will be understood to refer to and include the compound in its enol form (e.g., where Q is

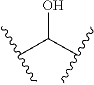 [SMILES: *C(*)O]

) unless context clearly indicates otherwise. The compounds may also exist as ring-chain tautomers, as discussed below.

Additional entactogenic compounds are described in PCT/US21/36479, and PCT/US21/51129 the entirety of each of which is incorporated by reference for all purposes.

In certain embodiments a combination is provided selected from:

TABLE 5

| | Tmax Agent 1 | Tmax Agent 2 |
|---|---|---|
| Active Agents | (minutes) | (minutes) |
| Agent 1 | about 30 minutes or | about 45, 60, or 75 |
| Agent 2 | less | |
| Agent 1 | about 45 minutes or | about 60, 75, or 90 |
| Agent 2 | less | |
| Agent 1 | about 60 minutes or | about 75, 90, or 105 |
| Agent 2 | less | |
| Agent 1 | about 75 minutes or | about 90, 105, or 130 |
| Agent 2 | less | |
| Agent 1- Dopamine releasing agent | about 30 minutes or | about 45, 60, or 75 |
| Agent 2- Entactogen | less | |
| Agent 1- Dopamine releasing agent | about 45 minutes or | about 60, 75, or 90 |
| Agent 2- Entactogen | less | |
| Agent 1- Dopamine releasing agent | about 60 minutes or | about 75, 90, or 105 |
| Agent 2- Entactogen | less | |
| Agent 1- Dopamine releasing agent | about 75 minutes or | about 90, 105, or 130 |
| Agent 2- Entactogen | less | |

wherein the combinations in the table above include pharmaceutically acceptable excipients to achieve the described release profile;
Agent 1 is selected from: amphetamine, methamphetamine, S-MDA, S-MDMA, S-MDE, S-BK-MDMA, RS-BK-MDMA, enantiomerically enriched 6-MBPB (more R), enantiomerically enriched 5-MBPB (more R), S-5-MAPB, 4-MMC, R-4-MMC, R-BK-MDMA, R-BK-MDEA, S-2-APB, S-5-APB, S-6-APB, S-BK-2-MAPB, S-BK-5-MAPB, S-BK-6-MAPB, S-BK-2-EAPB, S-BK-5-EAPB, S-BK-6-EAPB, S-BK-2-EBPB, S-BK-5-EBPB, S-BK-6-EBPB, S-2-EAPB, S-5-EAPB, S-6-EAPB, S-2-EBPB, S-aF-5-MAPB, R-aF-5-MAPB, S-aF-6-MAPB, R-aF-6-MAPB, S-5-EBPB, and S-6-EBPB,
Agent 2 is selected from: RS-MDMA, S-MDMA, RS-MDE, S-MDE, R-MDA, R-MDMA, R-MDE, R-BK-MDMA, enantiomerically enriched 6-MBPB (more S), enantiomerically enriched 5-MBPB (more S), R-5-MAPB, S-5-MAPB, S-4-MMC, S-MDE, enantiomerically enriched MDE (more S), S-BK-MDMA, S-BK-MDEA, MDAI, MMAI, R-2-APB, R-5-APB, R-6-APB, R-BK-2-MAPB, R-BK-5-MAPB, R-BK-6-MAPB, R-BK-2-EAPB, R-BK-5-EAPB, R-BK-6-EAPB, R-BK-2-EBPB, R-BK-5-EBPB, R-BK-6-EBPB, S-aF-5-MAPB, R-aF-5-MAPB, S-aF-6-MAPB, R-aF-6-MAPB, R-2-EAPB, R-5-EAPB, R-6-EAPB, R-2-EBPB, R-5-EBPB, and R-6-EBPB;
non-limiting examples of dopamine releasing agents include: amphetamine, methamphetamine, fencamfamine, phenmetrazine, 2-fluorophenmetrazine, 3-fluorophenmetrazine, metamnetamine (methyl[1-(5,6,7,8-tetrahydronaphthalen-2-yl)propan-2-yl]amine), naphthylaminopropane, 5-(2-Aminopropyl)indole, methcathinone, 2-methyl-methcathinone, 3-methyl-methcathinone, 4-methyl-methcathinone (4-MMC), 3-fluoroamphetamine, 3-fluoromethcathinone, 4-fluoroamphetamine, 4-fluoromethcathinone, 3-bromoamphetamine, 3-bromomethcathinone, 4-bromoamphetamine, 4-bromomethcathinone, N-methylamphetamine, N-benzyl-methamphetamine, 3-methylamphetamine, 4-methylamphetamine, N,4-dimethylamphetamine, 2-(Methylamino)-1-naphthalen-1-ylpropan-1-one, methylthioamphetamine, and N,N-dimethyl-thioamphetamine; and
non-limiting examples of entactogens include: MDMA, MDA (3,4-methylenedioxyamphetamine), MDAI (2H,5H,6H,7H-indeno[5,6-d][1,3]dioxol-6-amine), BK-MDMA (1-(2H-1,3-benzodioxol-5-yl)-2-(methylamino)propan-1-one), BK-MDEA (1-(2H-1,3-benzodioxol-5-yl)-2-(ethylamino)propan-1-one), MBDB ([1-(2H-1,3-benzodioxol-5-yl)butan-2-yl](methyl)amine), butylone (1-(2H-1,3-benzodioxol-5-yl)-2-(methylamino)butan-1-one), eutylone (1-(2H-1,3-benzodioxol-5-yl)-2-(ethylamino)butan-1-one), MDEA ([1-(2H-1,3-benzodioxol-5-yl)propan-2-yl](ethyl)amine), αMT (1-(1H-indol-3-yl)propan-2-amine), α,N-DMT ([1-(1H-indol-3-yl)propan-2-yl](methyl)amine), BK-NM-AMT (1-(1H-indol-3-yl)-2-(methylamino)propan-1-one), 5-F-NM-AMT ([1-(5-fluoro-1H-indol-3-yl)propan-2-yl](methyl)amine), BK-5-F-NM-AMT (1-(5-fluoro-1H-indol-3-yl)-2-(methylamino)propan-1-one), 2-APB (1-(1-benzofuran-2-yl)propan-2-amine), 5-APB (1-(1-benzofuran-5-yl)propan-2-amine), 6-APB ((1-(1-benzofuran-6-yl)propan-2-amine), APBI (5H,6H,7H-indeno[5,6-b]furan-6-amine), 2-MAPB ([1-(1-benzofuran-2-yl)propan-2-yl](methyl)amine), 5-MAPB ([1-(1-benzofuran-5-yl)propan-2-yl](methyl)amine), 6-MAPB ([1-(1-benzofuran-6-yl)propan-2-yl](methyl)amine), 2-EAPB ([1-(1-benzofuran-2-yl)propan-2-yl](ethyl)amine), 5-EAPB ([1-(1-benzofuran-2-yl)propan-5-yl](ethyl)amine), 6-EAPB ([1-(1-benzofuran-6-yl)propan-2-yl](ethyl)amine), 2-MBPB ([1-(1-benzofuran-2-yl)butan-2-yl](methyl)amine), 5-BPB ([1-(1-benzofuran-5-yl)butan-2-yl](methyl)amine), 6-MBPB ([1-(1-benzofuran-6-yl)butan-2-yl](methyl)amine), BK-5-MAPB (1-(1-benzofuran-5-yl)-2-(methylamino)propan-1-one), BK-2-MAPB (1-(1-benzofuran-2-yl)-2-(methylamino)propan-1-one), BK-6-EAPB (1-(1-benzofuran-6-yl)-2-(ethylamino)propan-1-one), BK-5-EAPB (1-(1-benzofuran-5-yl)-2-(ethylamino)propan-1-one), BK-2-EAPB (1-(1-benzofuran-2-yl)-2-(ethylamino)propan-1-one), FLEA (N-[1-(2H-1,3-benzodioxol-5-yl)propan-2-yl]-N-methylhydroxylamine), aF-5-MAPB ([1-(1-benzofuran-5-yl)-3-fluoropropan-2-yl](methyl)amine), aF-6-MAPB ([1-(1-benzofuran-6-yl)-3-fluoropropan-2-yl](methyl)amine) and MDOH (N-[1-(2H-1,3-benzodioxol-5-yl)propan-2-yl]hydroxylamine).

In certain embodiments the combination of the present invention is selected from:

TABLE 6

| | Tmax First Active | Tmax Second Active | Tmax Agent |
|---|---|---|---|
| Active Agents | Agent (minutes) | Agent (minutes) | 3 (minutes) |
| Agent 1 | immediate release, for | delayed release, for | — |
| 2-40 mg amphetamine | example an oral Tmax | example an oral Tmax | |
| Agent 2 | of about 45 to about 75 | of about 15 minutes | |
| 10-200 mg RS-MDMA | minutes or earlier | after First Active Agent | |
| | | oral Tmax | |
| Agent 1 | immediate release, for | delayed release, for | — |
| 2-40 mg methamphetamine | example an oral Tmax | example an oral Tmax | |
| Agent 2 | of about 45 to about 75 | of about 15 minutes | |
| 10-200 mg RS-MDMA | minutes or earlier | after First Active Agent | |
| | | oral Tmax | |
| Agent 1 | immediate release, for | delayed release, for | — |
| 2-40 mg amphetamine | example an oral Tmax | example an oral Tmax | |
| Agent 2 | of about 45 to about 75 | of about 15 minutes | |
| 10-200 mg S-MDMA | minutes or earlier | after First Active Agent | |
| | | oral Tmax | |
| Agent 1 | immediate release, for | delayed release, for | — |
| 2-40 mg methamphetamine | example an oral Tmax | example an oral Tmax | |
| Agent 2 | of about 45 to about 75 | of about 15 minutes | |
| 10-200 mg S-MDMA | minutes or earlier | after First Active Agent | |
| | | oral Tmax | |
| Agent 1 | immediate release, for | delayed release, for | — |
| | example an oral Tmax | example an oral Tmax | |
| 2-40 mg amphetamine | of about 45 to about 75 | of about 15 minutes | |
| Agent 2 | minutes or earlier | after First Active Agent | |
| 30-200 mg RS-MDE | | oral Tmax | |
| Agent 1 | immediate release, for | delayed release, for | — |
| 2-40 mg methamphetamine | example an oral Tmax | example an oral Tmax | |
| Agent 2 | of about 45 to about 75 | of about 15 minutes | |
| 30-200 mg RS-MDE | minutes or earlier | after First Active Agent | |
| | | oral Tmax | |
| Agent 1 | immediate release, for | delayed release, for | — |
| 2-40 mg amphetamine | example an oral Tmax | example an oral Tmax | |
| Agent 2 | of about 45 to about 75 | of about 15 minutes | |
| 30-200 mg S-MDE | minutes or earlier | after First Active Agent | |
| | | oral Tmax | |
| Agent 1 | immediate release, for | delayed release, for | — |
| 2-40 mg methamphetamine | example an oral Tmax | example an oral Tmax | |
| Agent 2 | of about 45 to about 75 | of about 15 minutes | |
| 30-200 mg S-MDE | minutes or earlier | after First Active Agent | |
| | | oral Tmax | |
| Agent 1 | immediate release, for | delayed release, for | — |
| 10-150 mg S-MDA | example an oral Tmax | example an oral Tmax | |
| Agent 2 | of about 45 to about 75 | of about 15 minutes | |
| 10-150 mg R-MDA | minutes or earlier | after First Active Agent | |
| | | oral Tmax | |
| Agent 1 | immediate release, for | delayed release, for | — |
| 10-180 mg S-MDMA | example an oral Tmax | example an oral Tmax | |
| Agent 2 | of about 45 to about 75 | of about 15 minutes | |
| 10-180 mg R-MDMA | minutes or earlier | after First Active Agent | |
| | | oral Tmax | |
| Agent 1 | immediate release, for | delayed release, for | — |
| 30-200 mg S-MDE | example an oral Tmax | example an oral Tmax | |
| Agent 2 | of about 45 to about 75 | of about 15 minutes | |
| 30-200 mg R-MDE | minutes or earlier | after First Active Agent | |
| | | oral Tmax | |
| Agent 1 | immediate release, for | delayed release, for | delayed |
| 2-40 mg amphetamine | example an oral Tmax | example an oral Tmax | release |
| Agent 2 | of about 45 to about 75 | of about 15 minutes | starting at |
| 10-200 mg RS-MDMA | minutes or earlier | after First Active Agent | about 120 to |
| Agent 3 | | oral Tmax | 360 min |
| 10-100 mg milnacipran or 20-40 | | | |
| mg citalopram or 20-60 mg | | | |
| duloxetine | | | |
| Agent 1 | immediate release, for | delayed release, for | delayed |
| 2-40 mg methamphetamine | example an oral Tmax | example an oral Tmax | release |
| Agent 2 | of about 45 to about 75 | of about 15 minutes | starting at |
| 10-200 mg RS-MDMA | minutes or earlier | after First Active Agent | about 120 to |
| Agent 3 | | oral Tmax | 360 min |
| 10-100 mg milnacipran or 20-40 | | | |
| mg citalopram or 20-60 mg | | | |
| duloxetine | | | |
| Agent 1 | immediate release, for | delayed release, for | delayed |
| 2-40 mg amphetamine | example an oral Tmax | example an oral Tmax | release |
| Agent 2 | of about 45 to about 75 | of about 15 minutes | starting at |
| 10-200 mg S-MDMA | minutes or earlier | after First Active Agent | about 120 to |
| Agent 3 | | oral Tmax | 360 min |
| 10-100 mg milnacipran or 20-40 | | | |
| mg citalopram or 20-60 mg | | | |
| duloxetine | | | |
| Agent 1 | immediate release, for | delayed release, for | delayed |
| 2-40 mg methamphetamine | example an oral Tmax | example an oral Tmax | release |
| Agent 2 | of about 45 to about 75 | of about 15 minutes | starting at |
| 10-200 mg S-MDMA | minutes or earlier | after First Active Agent | about 120 to |
| Agent 3 | | oral Tmax | 360 min |
| 10-100 mg milnacipran or 20-40 | | | |
| mg citalopram or 20-60 mg | | | |
| duloxetine | | | |
| Agent 1 | immediate release, for | delayed release, for | delayed |
| 2-40 mg amphetamine | example an oral Tmax | example an oral Tmax | release |
| Agent 2 | of about 45 to about 75 | of about 15 minutes | starting at |
| 30-200 mg RS-MDE | minutes or earlier | after First Active Agent | about 120 to |
| Agent 3 | | oral Tmax | 360 min |
| 10-100 mg milnacipran or 20-40 | | | |
| mg citalopram or 20-60 mg | | | |
| duloxetine | | | |
| Agent 1 | immediate release, for | delayed release, for | delayed |
| 2-40 mg methamphetamine | example an oral Tmax | example an oral Tmax | release |
| Agent 2 | of about 45 to about 75 | of about 15 minutes | starting at |
| 30-200 mg RS-MDE | minutes or earlier | after First Active Agent | about 120 to |
| Agent 3 | | oral Tmax | 360 min |
| 10-100 mg milnacipran or 20-40 | | | |
| mg citalopram or 20-60 mg | | | |
| duloxetine | | | |
| Agent 1 | immediate release, for | delayed release, for | delayed |
| 2-40 mg amphetamine | example an oral Tmax | example an oral Tmax | release |
| Agent 2 | of about 45 to about 75 | of about 15 minutes | starting at |
| 30-200 mg S-MDE | minutes or earlier | after First Active Agent | about 120 to |
| Agent 3 | | oral Tmax | 360 min |
| 10-100 mg milnacipran or 20-40 | | | |
| mg citalopram or 20-60 mg | | | |
| duloxetine | | | |
| Agent 1 | immediate release, for | delayed release, for | delayed |
| 2-40 mg methamphetamine | example an oral Tmax | example an oral Tmax | release |
| Agent 2 | of about 45 to about 75 | of about 15 minutes | starting at |
| 30-200 mg S-MDE | minutes or earlier | after First Active Agent | about 120 to |
| Agent 3 | | oral Tmax | 360 min |
| 10-100 mg milnacipran or 20-40 | | | |
| mg citalopram or 20-60 mg | | | |
| duloxetine | | | |
| Agent 1 | immediate release, for | delayed release, for | delayed |
| 10-150 mg S-MDA | example an oral Tmax | example an oral Tmax | release |
| Agent 2 | of about 45 to about 75 | of about 15 minutes | starting at |
| 10-150 mg R-MDA | minutes or earlier | after First Active Agent | about 120 to |
| Agent 3 | | oral Tmax | 360 min |
| 10-100 mg milnacipran or 20-40 | | | |
| mg citalopram or 20-60 mg | | | |
| duloxetine | | | |
| Agent 1 | immediate release, for | delayed release, for | delayed |
| 10-180 mg S-MDMA | example an oral Tmax | example an oral Tmax | release |
| Agent 2 | of about 45 to about 75 | of about 15 minutes | starting at |
| 10-180 mg R-MDMA | minutes or earlier | after First Active Agent | about 120 to |
| Agent 3 | | oral Tmax | 360 min |
| 10-100 mg milnacipran or 20-40 | | | |
| mg citalopram or 20-60 mg | | | |
| duloxetine | | | |
| Agent 1 | immediate release, for | delayed release, for | delayed |
| 30-200 mg S-MDE | example an oral Tmax | example an oral Tmax | release |
| Agent 2 | of about 45 to about 75 | of about 15 minutes | starting at |
| 30-200 mg R-MDE | minutes or earlier | after First Active Agent | about 120 to |
| Agent 3 | | oral Tmax | 360 min |
| 10-100 mg milnacipran or 20-40 | | | |
| mg citalopram or 20-60 mg | | | |
| duloxetine | | | |
| Agent 1 | immediate release, for | delayed release, for | — |
| 10-500 mg S-BK-MDMA | example an oral Tmax | example an oral Tmax | |
| Agent 2 | of about 45 to about 75 | of about 15 minutes | |
| 10-500 mg R-BK-MDMA | minutes or earlier | after First Active Agent | |
| | | oral Tmax | |
| Agent 1 | immediate release, for | delayed release, for | — |
| 25-500 mg BK-MDMA | example an oral Tmax | example an oral Tmax | |
| Agent 2 | of about 45 to about 75 | of about 15 minutes | |
| | minutes or earlier | | |
| 10-200 mg RS-MDMA | | after First Active Agent | |
| | | oral Tmax | |
| Agent 1 | immediate release, for | delayed release, for | — |
| 5-120 mg enantiomerically | example an oral Tmax | example an oral Tmax | |
| enriched 6-MBPB (more R) | of about 45 to about 75 | of about 15 minutes | |
| Agent 2 | minutes or earlier | after First Active Agent | |
| 5-120 mg enantiomerically | | oral Tmax | |
| enriched 6-MBPB (more S) | | | |
| Agent 1 | immediate release, for | delayed release, for | — |
| 5-120 mg enantiomerically | example an oral Tmax | example an oral Tmax | |
| enriched 5-MBPB (more R) | of about 45 to about 75 | of about 15 minutes | |
| Agent 2 | minutes or earlier | after First Active Agent | |
| 5-120 mg enantiomerically | | oral Tmax | |
| enriched 5-MBPB (more S) | | | |
| Agent 1 | immediate release, for | delayed release, for | — |
| 5-120 mg S-5-MAPB | example an oral Tmax | example an oral Tmax | |
| Agent 2 | of about 45 to about 75 | of about 15 minutes | |
| 5-120 mg R-5-MAPB | minutes or earlier | after First Active Agent | |
| | | oral Tmax | |
| Agent 1 | immediate release, for | delayed release, for | — |
| 5-120 mg S-5-MAPB | example an oral Tmax | example an oral Tmax | |
| Agent 2 | of about 45 to about 75 | of about 15 minutes | |
| 10-200 mg RS-MDMA | minutes or earlier | after First Active Agent | |
| | | oral Tmax | |
| Agent 1 | immediate release, for | delayed release, for | — |
| 2-40 mg amphetamine | example an oral Tmax | example an oral Tmax | |
| Agent 2 | of about 45 to about 75 | of about 15 minutes | |
| 5-120 mg S-5-MAPB | minutes or earlier | after First Active Agent | |
| | | oral Tmax | |
| Agent 1 | immediate release, for | delayed release, for | delayed |
| 2-40 mg amphetamine | example an oral Tmax | example an oral Tmax | release |
| Agent 2 | of about 45 to about 75 | of about 15 minutes | starting at |
| 5-120 mg S-5-MAPB | minutes or earlier | after First Active Agent | about 120 to |
| Agent 3 | | oral Tmax | 360 min |
| 10-100 mg milnacipran or 20-40 | | | |
| mg citalopram or 20-60 mg | | | |
| duloxetine | | | |
| Agent 1 | immediate release, for | delayed release, for | — |
| 2-40 mg methamphetamine | example an oral Tmax | example an oral Tmax | |
| Agent 2 | of about 45 to about 75 | of about 15 minutes | |
| 5-120 mg S-5-MAPB | minutes or earlier | after First Active Agent | |
| | | oral Tmax | |
| Agent 1 | immediate release, for | delayed release, for | — |
| 5-400 mg 4-MMC | example an oral Tmax | example an oral Tmax | |
| Agent 2 | of about 45 to about 75 | of about 15 minutes | |
| 10-200 mg RS-MDMA | minutes or earlier | after First Active Agent | |
| | | oral Tmax | |
| Agent 1 | immediate release, for | delayed release, for | — |
| 5-400 mg 4-MMC | example an oral Tmax | example an oral Tmax | |
| Agent 2 | of about 45 to about 75 | of about 15 minutes | |
| 10-200 mg RS-MDMA | minutes or earlier | after First Active Agent | |
| | | oral Tmax | |
| Agent 1 | immediate release, for | delayed release, for | — |
| 5-400 mg R-4-MMC | example an oral Tmax | example an oral Tmax | |
| Agent 2 | of about 45 to about 75 | of about 15 minutes | |
| 5-400 mg S-4-MMC | minutes or earlier | after First Active Agent | |
| | | oral Tmax | |
| Agent 1 | immediate release, for | delayed release, for | — |
| 2-40 mg amphetamine | example an oral Tmax | example an oral Tmax | |
| Agent 2 | of about 45 to about 75 | of about 15 minutes | |
| 30-200 mg S-MDE | minutes or earlier | after First Active Agent | |
| | | oral Tmax | |
| Agent 1 | immediate release, for | delayed release, for | — |
| 2-40 mg amphetamine | example an oral Tmax | example an oral Tmax | |
| Agent 2 | of about 45 to about 75 | of about 15 minutes | |
| 30-200 mg enantiomerically | minutes or earlier | after First Active Agent | |
| enriched MDE (more S) | | oral Tmax | |
| Agent 1 | immediate release, for | delayed release, for | — |
| 5-300 mg R-BK-MDMA | example an oral Tmax | example an oral Tmax | |
| Agent 2 | of about 45 to about 75 | of about 15 minutes | |
| 5-300 mg S-BK-MDMA | minutes or earlier | after First Active Agent | |
| | | oral Tmax | |
| Agent 1 | immediate release, for | delayed release, for | — |
| 5-300 mg R-BK-MDEA | example an oral Tmax | example an oral Tmax | |
| Agent 2 | of about 45 to about 75 | of about 15 minutes | |
| 5-300 mg S-BK-MDEA | minutes or earlier | after First Active Agent | |
| | | oral Tmax | |
| Agent 1 | immediate release, for | delayed release, for | — |
| 2-40 mg amphetamine | example an oral Tmax | example an oral Tmax | |
| Agent 2 | of about 45 to about 75 | of about 15 minutes | |
| 50-300 mg MDAI | minutes or earlier | after First Active Agent | |
| | | oral Tmax | |
| Agent 1 | immediate release, for | delayed release, for | — |
| 2-40 mg amphetamine | example an oral Tmax | example an oral Tmax | |
| Agent 2 | of about 45 to about 75 | of about 15 minutes | |
| 5-60 mg MMAI | minutes or earlier | after First Active Agent | |
| | | oral Tmax | |
| Agent 1 | immediate release, for | delayed release, for | — |
| 5-100 mg S-2-APB | example an oral Tmax | example an oral Tmax | |
| Agent 2 | of about 45 to about 75 | of about 15 minutes | |
| 5-100 mg R-2-APB | minutes or earlier | after First Active Agent | |
| | | oral Tmax | |
| Agent 1 | immediate release, for | delayed release, for | — |
| 5-100 mg S-5-APB | example an oral Tmax | example an oral Tmax | |
| Agent 2 | of about 45 to about 75 | of about 15 minutes | |
| 5-100 mg R-5-APB | minutes or earlier | after First Active Agent | |
| | | oral Tmax | |
| Agent 1 | immediate release, for | delayed release, for | — |
| 5-100 mg S-6-APB | example an oral Tmax | example an oral Tmax | |
| Agent 2 | of about 45 to about 75 | of about 15 minutes | |
| 5-100 mg R-6-APB | minutes or earlier | after First Active Agent | |
| | | oral Tmax | |
| Agent 1 | immediate release, for | delayed release, for | — |
| 5-300 mg S-BK-2-MAPB | example an oral Tmax | example an oral Tmax | |
| Agent 2 | of about 45 to about 75 | of about 15 minutes | |
| 5-300 mg R-BK-2-MAPB | minutes or earlier | after First Active Agent | |
| | | oral Tmax | |
| Agent 1 | immediate release, for | delayed release, for | — |
| 5-150 mg S-BK-5-MAPB | example an oral Tmax | example an oral Tmax | |
| Agent 2 | of about 45 to about 75 | of about 15 minutes | |
| 5-150 mg R-BK-5-MAPB | minutes or earlier | after First Active Agent | |
| | | oral Tmax | |
| Agent 1 | immediate release, for | delayed release, for | — |
| 5-300 mg S-BK-6-MAPB | example an oral Tmax | example an oral Tmax | |
| Agent 2 | of about 45 to about 75 | of about 15 minutes | |
| 5-300 mg R-BK-6-MAPB | minutes or earlier | after First Active Agent | |
| | | oral Tmax | |
| Agent 1 | immediate release, for | delayed release, for | — |
| 5-300 mg S-BK-2-EAPB | example an oral Tmax | example an oral Tmax | |
| Agent 2 | of about 45 to about 75 | of about 15 minutes | |
| 5-300 mg R-BK-2-EAPB | minutes or earlier | after First Active Agent | |
| | | oral Tmax | |
| Agent 1 | immediate release, for | delayed release, for | — |
| 5-300 mg S-BK-5-EAPB | example an oral Tmax | example an oral Tmax | |
| Agent 2 | of about 45 to about 75 | of about 15 minutes | |
| 5-300 mg R-BK-5-EAPB | minutes or earlier | after First Active Agent | |
| | | oral Tmax | |
| Agent 1 | immediate release, for | delayed release, for | — |
| 5-300 mg S-BK-6-EAPB | example an oral Tmax | example an oral Tmax | |
| Agent 2 | of about 45 to about 75 | of about 15 minutes | |
| 5-300 mg R-BK-6-EAPB | minutes or earlier | after First Active Agent | |
| | | oral Tmax | |
| Agent 1 | immediate release, for | delayed release, for | — |
| 5-300 mg S-BK-2-EBPB | example an oral Tmax | example an oral Tmax | |
| Agent 2 | of about 45 to about 75 | of about 15 minutes | |
| 5-300 mg R-BK-2-EBPB | minutes or earlier | after First Active Agent | |
| | | oral Tmax | |
| Agent 1 | immediate release, for | delayed release, for | — |
| 5-300 mg S-BK-5-EBPB | example an oral Tmax | example an oral Tmax | |
| Agent 2 | of about 45 to about 75 | of about 15 minutes | |
| 5-300 mg R-BK-5-EBPB | minutes or earlier | after First Active Agent | |
| | | oral Tmax | |
| Agent 1 | immediate release, for | delayed release, for | — |
| 5-300 mg S-BK-6-EBPB | example an oral Tmax | example an oral Tmax | |
| Agent 2 | of about 45 to about 75 | of about 15 minutes | |
| 5-300 mg R-BK-6-EBPB | minutes or earlier | after First Active Agent | |
| | | oral Tmax | |
| Agent 1 | immediate release, for | delayed release, for | — |
| 5-300 mg S-2-EAPB | example an oral Tmax | example an oral Tmax | |
| Agent 2 | of about 45 to about 75 | of about 15 minutes | |
| 5-300 mg R-2-EAPB | minutes or earlier | after First Active Agent | |
| | | oral Tmax | |
| Agent 1 | immediate release, for | delayed release, for | — |
| 5-300 mg S-5-EAPB | example an oral Tmax | example an oral Tmax | |
| Agent 2 | of about 45 to about 75 | of about 15 minutes | |
| 5-300 mg R-5-EAPB | minutes or earlier | after First Active Agent | |
| | | oral Tmax | |
| Agent 1 | immediate release, for | delayed release, for | — |
| 5-300 mg S-6-EAPB | example an oral Tmax | example an oral Tmax | |
| Agent 2 | of about 45 to about 75 | of about 15 minutes | |
| 5-300 mg R-6-EAPB | minutes or earlier | after First Active Agent | |
| | | oral Tmax | |
| Agent 1 | immediate release, for | delayed release, for | — |
| 5-300 mg S-2-EBPB | example an oral Tmax | example an oral Tmax | |
| Agent 2 | of about 45 to about 75 | of about 15 minutes | |
| 5-300 mg R-2-EBPB | minutes or earlier | after First Active Agent | |
| | | oral Tmax | |
| Agent 1 | immediate release, for | delayed release, for | — |
| 5-300 mg S-5-EBPB | example an oral Tmax | example an oral Tmax | |
| Agent 2 | of about 45 to about 75 | of about 15 minutes | |
| 5-300 mg R-5-EBPB | minutes or earlier | after First Active Agent | |
| | | oral Tmax | |
| Agent 1 | immediate release, for | delayed release, for | — |
| 5-300 mg S-6-EBPB | example an oral Tmax | example an oral Tmax | |
| Agent 2 | of about 45 to about 75 | of about 15 minutes | |
| 5-300 mg R-6-EBPB | minutes or earlier | after First Active Agent | |
| | | oral Tmax | |

In certain embodiments Agent 1 is the first active agent with lower Tmax as it is formulated for immediate release, and Agent 2 is the second active agent higher Tmax as it is formulated for delayed release.

In certain alternate embodiments Agent 2 is the first active agent with lower Tmax as it is formulated for immediate release, and Agent 1 is the second active agent higher Tmax as it is formulated for delayed release.

TABLE 7
In other embodiments the combination of the present invention is selected from:

| | | | Tpeak |
|---|---|---|---|
| | Tpeak First Active | Tpeak Second Active | Agent 3 |
| Active Agents | Agent (minutes) | Agent (minutes) | (minutes) |
| Agent 1 | immediate release, for | delayed release, for | — |
| 2-40 mg amphetamine | example an oral Tpeak | example an oral Tpeak | |
| Agent 2 | of about 45 to about 75 | of about 15 minutes | |
| 10-200 mg RS-MDMA | minutes or earlier | after First Active Agent | |
| | | oral Tpeak | |
| Agent 1 | immediate release, for | delayed release, for | — |
| 2-40 mg methamphetamine | example an oral Tpeak | example an oral Tpeak | |
| Agent 2 | of about 45 to about 75 | of about 15 minutes | |
| 10-200 mg RS-MDMA | minutes or earlier | after First Active Agent | |
| | | oral Tpeak | |
| Agent 1 | immediate release, for | delayed release, for | — |
| 2-40 mg amphetamine | example an oral Tpeak | example an oral Tpeak | |
| Agent 2 | of about 45 to about 75 | of about 15 minutes | |
| 10-200 mg S-MDMA | minutes or earlier | after First Active Agent | |
| | | oral Tpeak | |
| Agent 1 | immediate release, for | delayed release, for | — |
| 2-40 mg methamphetamine | example an oral Tpeak | example an oral Tpeak | |
| Agent 2 | of about 45 to about 75 | of about 15 minutes | |
| 10-200 mg S-MDMA | minutes or earlier | after First Active Agent | |
| | | oral Tpeak | |
| Agent 1 | immediate release, for | delayed release, for | — |
| 2-40 mg amphetamine | example an oral Tpeak | example an oral Tpeak | |
| Agent 2 | of about 45 to about 75 | of about 15 minutes | |
| 30-200 mg RS-MDE | minutes or earlier | after First Active Agent | |
| | | oral Tpeak | |
| Agent 1 | immediate release, for | delayed release, for | — |
| 2-40 mg methamphetamine | example an oral Tpeak | example an oral Tpeak | |
| Agent 2 | of about 45 to about 75 | of about 15 minutes | |
| 30-200 mg RS-MDE | minutes or earlier | after First Active Agent | |
| | | oral Tpeak | |
| Agent 1 | immediate release, for | delayed release, for | — |
| 2-40 mg amphetamine | example an oral Tpeak | example an oral Tpeak | |
| Agent 2 | of about 45 to about 75 | of about 15 minutes | |
| 30-200 mg S-MDE | minutes or earlier | after First Active Agent | |
| | | oral Tpeak | |
| Agent 1 | immediate release, for | delayed release, for | — |
| 2-40 mg methamphetamine | example an oral Tpeak | example an oral Tpeak | |
| Agent 2 | of about 45 to about 75 | of about 15 minutes | |
| 30-200 mg S-MDE | minutes or earlier | after First Active Agent | |
| | | oral Tpeak | |
| Agent 1 | immediate release, for | delayed release, for | — |
| 10-150 mg S-MDA | example an oral Tpeak | example an oral Tpeak | |
| Agent 2 | of about 45 to about 75 | of about 15 minutes | |
| 10-150 mg R-MDA | minutes or earlier | after First Active Agent | |
| | | oral Tpeak | |
| Agent 1 | immediate release, for | delayed release, for | — |
| 10-180 mg S-MDMA | example an oral Tpeak | example an oral Tpeak | |
| Agent 2 | of about 45 to about 75 | of about 15 minutes | |
| 10-180 mg R-MDMA | minutes or earlier | after First Active Agent | |
| | | oral Tpeak | |
| Agent 1 | immediate release, for | delayed release, for | — |
| 30-200 mg S-MDE | example an oral Tpeak | example an oral Tpeak | |
| Agent 2 | of about 45 to about 75 | of about 15 minutes | |
| 30-200 mg R-MDE | minutes or earlier | after First Active Agent | |
| | | oral Tpeak | |
| Agent 1 | immediate release, for | delayed release, for | delayed |
| 2-40 mg amphetamine | example an oral Tpeak | example an oral Tpeak | release |
| Agent 2 | of about 45 to about 75 | of about 15 minutes | starting at |
| 10-200 mg RS-MDMA | minutes or earlier | after First Active Agent | about 120 to |
| Agent 3 | | oral Tpeak | 360 min |
| 10-100 mg milnacipran or 20-40 | | | |
| mg citalopram or 20-60 mg | | | |
| duloxetine | | | |
| Agent 1 | immediate release, for | delayed release, for | delayed |
| 2-40 mg methamphetamine | example an oral Tpeak | example an oral Tpeak | release |
| Agent 2 | of about 45 to about 75 | of about 15 minutes | starting at |
| 10-200 mg RS-MDMA | minutes or earlier | after First Active Agent | about 120 to |
| Agent 3 | | oral Tpeak | 360 min |
| 10-100 mg milnacipran or 20-40 | | | |
| mg citalopram or 20-60 mg | | | |
| duloxetine | | | |
| Agent 1 | immediate release, for | delayed release, for | delayed |
| 2-40 mg amphetamine | example an oral Tpeak | example an oral Tpeak | release |
| Agent 2 | of about 45 to about 75 | of about 15 minutes | starting at |
| 10-200 mg S-MDMA | minutes or earlier | after First Active Agent | about 120 to |
| Agent 3 | | oral Tpeak | 360 min |
| 10-100 mg milnacipran or 20-40 | | | |
| mg citalopram or 20-60 mg | | | |
| duloxetine | | | |
| Agent 1 | immediate release, for | delayed release, for | delayed |
| 2-40 mg methamphetamine | example an oral Tpeak | example an oral Tpeak | release |
| Agent 2 | of about 45 to about 75 | of about 15 minutes | starting at |
| 10-200 mg S-MDMA | minutes or earlier | after First Active Agent | about 120 to |
| Agent 3 | | oral Tpeak | 360 min |
| 10-100 mg milnacipran or 20-40 | | | |
| mg citalopram or 20-60 mg | | | |
| duloxetine | | | |
| Agent 1 | immediate release, for | delayed release, for | delayed |
| 2-40 mg amphetamine | example an oral Tpeak | example an oral Tpeak | release |
| Agent 2 | of about 45 to about 75 | of about 15 minutes | starting at |
| 30-200 mg RS-MDE | minutes or earlier | after First Active Agent | about 120 to |
| Agent 3 | | oral Tpeak | 360 min |
| 10-100 mg milnacipran or 20-40 | | | |
| mg citalopram or 20-60 mg | | | |
| duloxetine | | | |
| Agent 1 | immediate release, for | delayed release, for | delayed |
| 2-40 mg methamphetamine | example an oral Tpeak | example an oral Tpeak | release |
| Agent 2 | of about 45 to about 75 | of about 15 minutes | starting at |
| 30-200 mg RS-MDE | minutes or earlier | after First Active Agent | about 120 to |
| Agent 3 | | oral Tpeak | 360 min |
| 10-100 mg milnacipran or 20-40 | | | |
| mg citalopram or 20-60 mg | | | |
| duloxetine | | | |
| Agent 1 | immediate release, for | delayed release, for | delayed |
| 2-40 mg amphetamine | example an oral Tpeak | example an oral Tpeak | release |
| Agent 2 | of about 45 to about 75 | of about 15 minutes | starting at |
| 30-200 mg S-MDE | minutes or earlier | after First Active Agent | about 120 to |
| Agent 3 | | oral Tpeak | 360 min |
| 10-100 mg milnacipran or 20-40 | | | |
| mg citalopram or 20-60 mg | | | |
| duloxetine | | | |
| Agent 1 | immediate release, for | delayed release, for | delayed |
| 2-40 mg methamphetamine | example an oral Tpeak | example an oral Tpeak | release |
| Agent 2 | of about 45 to about 75 | of about 15 minutes | starting at |
| 30-200 mg S-MDE | minutes or earlier | after First Active Agent | about 120 to |
| Agent 3 | | oral Tpeak | 360 min |
| 10-100 mg milnacipran or 20-40 | | | |
| mg citalopram or 20-60 mg | | | |
| duloxetine | | | |
| Agent 1 | immediate release, for | delayed release, for | delayed |
| 10-150 mg S-MDA | example an oral Tpeak | example an oral Tpeak | release |
| Agent 2 | of about 45 to about 75 | of about 15 minutes | starting at |
| 10-150 mg R-MDA | minutes or earlier | after First Active Agent | about 120 to |
| Agent 3 | | oral Tpeak | 360 min |
| 10-100 mg milnacipran or 20-40 | | | |
| mg citalopram or 20-60 mg | | | |
| duloxetine | | | |
| Agent 1 | immediate release, for | delayed release, for | delayed |
| 10-180 mg S-MDMA | example an oral Tpeak | example an oral Tpeak | release |
| Agent 2 | of about 45 to about 75 | of about 15 minutes | starting at |
| 10-180 mg R-MDMA | minutes or earlier | after First Active Agent | about 120 to |
| Agent 3 | | oral Tpeak | 360 min |
| 10-100 mg milnacipran or 20-40 | | | |
| mg citalopram or 20-60 mg | | | |
| duloxetine | | | |
| Agent 1 | immediate release, for | delayed release, for | delayed |
| 30-200 mg S-MDE | example an oral Tpeak | example an oral Tpeak | release |
| Agent 2 | of about 45 to about 75 | of about 15 minutes | starting at |
| 30-200 mg R-MDE | minutes or earlier | after First Active Agent | about 120 to |
| Agent 3 | | oral Tpeak | 360 min |
| 10-100 mg milnacipran or 20-40 | | | |
| mg citalopram or 20-60 mg | | | |
| duloxetine | | | |
| Agent 1 | immediate release, for | delayed release, for | — |
| 10-500 mg S-BK-MDMA | example an oral Tpeak | example an oral Tpeak | |
| Agent 2 | of about 45 to about 75 | of about 15 minutes | |
| 10-500 mg R-BK-MDMA | minutes or earlier | after First Active Agent | |
| | | oral Tpeak | |
| Agent 1 | immediate release, for | delayed release, for | — |
| 25-500 mg BK-MDMA | example an oral Tpeak | example an oral Tpeak | |
| Agent 2 | of about 45 to about 75 | of about 15 minutes | |
| 10-200 mg RS-MDMA | minutes or earlier | after First Active Agent | |
| | | oral Tpeak | |
| Agent 1 | immediate release, for | delayed release, for | — |
| 5-120 mg enantiomerically | example an oral Tpeak | example an oral Tpeak | |
| enriched 6-MBPB (more R) | of about 45 to about 75 | of about 15 minutes | |
| Agent 2 | minutes or earlier | after First Active Agent | |
| 5-120 mg enantiomerically | | oral Tpeak | |
| enriched 6-MBPB (more S) | | | |
| Agent 1 | immediate release, for | delayed release, for | — |
| 5-120 mg enantiomerically | example an oral Tpeak | example an oral Tpeak | |
| enriched 5-MBPB (more R) | of about 45 to about 75 | of about 15 minutes | |
| Agent 2 | minutes or earlier | after First Active Agent | |
| 5-120 mg enantiomerically | | oral Tpeak | |
| enriched 5-MBPB (more S) | | | |
| Agent 1 | immediate release, for | delayed release, for | — |
| 5-120 mg S-5-MAPB | example an oral Tpeak | example an oral Tpeak | |
| Agent 2 | of about 45 to about 75 | of about 15 minutes | |
| 5-120 mg R-5-MAPB | minutes or earlier | after First Active Agent | |
| | | oral Tpeak | |
| Agent 1 | immediate release, for | delayed release, for | — |
| 5-120 mg S-5-MAPB | example an oral Tpeak | example an oral Tpeak | |
| Agent 2 | of about 45 to about 75 | of about 15 minutes | |
| 10-200 mg RS-MDMA | minutes or earlier | after First Active Agent | |
| | | oral Tpeak | |
| Agent 1 | immediate release, for | delayed release, for | — |
| 2-40 mg amphetamine | example an oral Tpeak | example an oral Tpeak | |
| Agent 2 | of about 45 to about 75 | of about 15 minutes | |
| 5-120 mg S-5-MAPB | minutes or earlier | after First Active Agent | |
| | | oral Tpeak | |
| Agent 1 | immediate release, for | delayed release, for | delayed |
| 2-40 mg amphetamine | example an oral Tpeak | example an oral Tpeak | release |
| Agent 2 | of about 45 to about 75 | of about 15 minutes | starting at |
| 5-120 mg S-5-MAPB | minutes or earlier | after First Active Agent | about 120 to |
| Agent 3 | | oral Tpeak | 360 min |
| 10-100 mg milnacipran or 20-40 | | | |
| mg citalopram or 20-60 mg | | | |
| duloxetine | | | |
| Agent 1 | immediate release, for | delayed release, for | — |
| 2-40 mg methamphetamine | example an oral Tpeak | example an oral Tpeak | |
| Agent 2 | of about 45 to about 75 | of about 15 minutes | |
| 5-120 mg S-5-MAPB | minutes or earlier | after First Active Agent | |
| | | oral Tpeak | |
| Agent 1 | immediate release, for | delayed release, for | — |
| 5-400 mg 4-MMC | example an oral Tpeak | example an oral Tpeak | |
| Agent 2 | of about 45 to about 75 | of about 15 minutes | |
| 10-200 mg RS-MDMA | minutes or earlier | after First Active Agent | |
| | | oral Tpeak | |
| Agent 1 | immediate release, for | delayed release, for | — |
| 5-400 mg 4-MMC | example an oral Tpeak | example an oral Tpeak | |
| Agent 2 | of about 45 to about 75 | of about 15 minutes | |
| 10-200 mg RS-MDMA | minutes or earlier | after First Active Agent | |
| | | oral Tpeak | |
| Agent 1 | immediate release, for | delayed release, for | — |
| 5-400 mg R-4-MMC | example an oral Tpeak | example an oral Tpeak | |
| Agent 2 | of about 45 to about 75 | of about 15 minutes | |
| 5-400 mg S-4-MMC | minutes or earlier | after First Active Agent | |
| | | oral Tpeak | |
| Agent 1 | immediate release, for | delayed release, for | — |
| 2-40 mg amphetamine | example an oral Tpeak | example an oral Tpeak | |
| Agent 2 | of about 45 to about 75 | of about 15 minutes | |
| 30-200 mg S-MDE | minutes or earlier | after First Active Agent | |
| | | oral Tpeak | |
| Agent 1 | immediate release, for | delayed release, for | — |
| 2-40 mg amphetamine | example an oral Tpeak | example an oral Tpeak | |
| Agent 2 | of about 45 to about 75 | of about 15 minutes | |
| 30-200 mg enantiomerically | minutes or earlier | after First Active Agent | |
| enriched MDE (more S) | | oral Tpeak | |
| Agent 1 | immediate release, for | delayed release, for | — |
| 5-300 mg R-BK-MDMA | example an oral Tpeak | example an oral Tpeak | |
| Agent 2 | of about 45 to about 75 | of about 15 minutes | |
| 5-300 mg S-BK-MDMA | minutes or earlier | after First Active Agent | |
| | | oral Tpeak | |
| Agent 1 | immediate release, for | delayed release, for | — |
| 5-300 mg R-BK-MDEA | example an oral Tpeak | example an oral Tpeak | |
| Agent 2 | of about 45 to about 75 | of about 15 minutes | |
| 5-300 mg S-BK-MDEA | minutes or earlier | after First Active Agent | |
| | | oral Tpeak | |
| Agent 1 | immediate release, for | delayed release, for | — |
| 2-40 mg amphetamine | example an oral Tpeak | example an oral Tpeak | |
| Agent 2 | of about 45 to about 75 | of about 15 minutes | |
| 50-300 mg MDAI | minutes or earlier | after First Active Agent | |
| | | oral Tpeak | |
| Agent 1 | immediate release, for | delayed release, for | — |
| 2-40 mg amphetamine | example an oral Tpeak | example an oral Tpeak | |
| Agent 2 | of about 45 to about 75 | of about 15 minutes | |
| 5-60 mg MMAI | minutes or earlier | after First Active Agent | |
| | | oral Tpeak | |
| Agent 1 | immediate release, for | delayed release, for | — |
| 5-100 mg S-2-APB | example an oral Tpeak | example an oral Tpeak | |
| Agent 2 | of about 45 to about 75 | of about 15 minutes | |
| 5-100 mg R-2-APB | minutes or earlier | after First Active Agent | |
| | | oral Tpeak | |
| Agent 1 | immediate release, for | delayed release, for | — |
| 5-100 mg S-5-APB | example an oral Tpeak | example an oral Tpeak | |
| Agent 2 | of about 45 to about 75 | of about 15 minutes | |
| 5-100 mg R-5-APB | minutes or earlier | after First Active Agent | |
| | | oral Tpeak | |
| Agent 1 | immediate release, for | delayed release, for | — |
| 5-100 mg S-6-APB | example an oral Tpeak | example an oral Tpeak | |
| Agent 2 | of about 45 to about 75 | of about 15 minutes | |
| 5-100 mg R-6-APB | minutes or earlier | after First Active Agent | |
| | | oral Tpeak | |
| Agent 1 | immediate release, for | delayed release, for | — |
| 5-300 mg S-BK-2-MAPB | example an oral Tpeak | example an oral Tpeak | |
| Agent 2 | of about 45 to about 75 | of about 15 minutes | |
| 5-300 mg R-BK-2-MAPB | minutes or earlier | after First Active Agent | |
| | | oral Tpeak | |
| Agent 1 | immediate release, for | delayed release, for | — |
| 5-150 mg S-BK-5-MAPB | example an oral Tpeak | example an oral Tpeak | |
| Agent 2 | of about 45 to about 75 | of about 15 minutes | |
| 5-150 mg R-BK-5-MAPB | minutes or earlier | after First Active Agent | |
| | | oral Tpeak | |
| Agent 1 | immediate release, for | delayed release, for | — |
| 5-300 mg S-BK-6-MAPB | example an oral Tpeak | example an oral Tpeak | |
| Agent 2 | of about 45 to about 75 | of about 15 minutes | |
| 5-300 mg R-BK-6-MAPB | minutes or earlier | after First Active Agent | |
| | | oral Tpeak | |
| Agent 1 | immediate release, for | delayed release, for | — |
| 5-300 mg S-BK-2-EAPB | example an oral Tpeak | example an oral Tpeak | |
| Agent 2 | of about 45 to about 75 | of about 15 minutes | |
| 5-300 mg R-BK-2-EAPB | minutes or earlier | after First Active Agent | |
| | | oral Tpeak | |
| Agent 1 | immediate release, for | delayed release, for | — |
| 5-300 mg S-BK-5-EAPB | example an oral Tpeak | example an oral Tpeak | |
| Agent 2 | of about 45 to about 75 | of about 15 minutes | |
| 5-300 mg R-BK-5-EAPB | minutes or earlier | after First Active Agent | |
| | | oral Tpeak | |
| Agent 1 | immediate release, for | delayed release, for | — |
| 5-300 mg S-BK-6-EAPB | example an oral Tpeak | example an oral Tpeak | |
| Agent 2 | of about 45 to about 75 | of about 15 minutes | |
| 5-300 mg R-BK-6-EAPB | minutes or earlier | after First Active Agent | |
| | | oral Tpeak | |
| Agent 1 | immediate release, for | delayed release, for | — |
| 5-300 mg S-BK-2-EBPB | example an oral Tpeak | example an oral Tpeak | |
| Agent 2 | of about 45 to about 75 | of about 15 minutes | |
| 5-300 mg R-BK-2-EBPB | minutes or earlier | after First Active Agent | |
| | | oral Tpeak | |
| Agent 1 | immediate release, for | delayed release, for | — |
| 5-300 mg S-BK-5-EBPB | example an oral Tpeak | example an oral Tpeak | |
| Agent 2 | of about 45 to about 75 | of about 15 minutes | |
| 5-300 mg R-BK-5-EBPB | minutes or earlier | after First Active Agent | |
| | | oral Tpeak | |
| Agent 1 | immediate release, for | delayed release, for | — |
| 5-300 mg S-BK-6-EBPB | example an oral Tpeak | example an oral Tpeak | |
| Agent 2 | of about 45 to about 75 | of about 15 minutes | |
| 5-300 mg R-BK-6-EBPB | minutes or earlier | after First Active Agent | |
| | | oral Tpeak | |
| Agent 1 | immediate release, for | delayed release, for | — |
| 5-300 mg S-2-EAPB | example an oral Tpeak | example an oral Tpeak | |
| Agent 2 | of about 45 to about 75 | of about 15 minutes | |
| 5-300 mg R-2-EAPB | minutes or earlier | after First Active Agent | |
| | | oral Tpeak | |
| Agent 1 | immediate release, for | delayed release, for | — |
| 5-300 mg S-5-EAPB | example an oral Tpeak | example an oral Tpeak | |
| Agent 2 | of about 45 to about 75 | of about 15 minutes | |
| 5-300 mg R-5-EAPB | minutes or earlier | after First Active Agent | |
| | | oral Tpeak | |
| Agent 1 | immediate release, for | delayed release, for | — |
| 5-300 mg S-6-EAPB | example an oral Tpeak | example an oral Tpeak | |
| Agent 2 | of about 45 to about 75 | of about 15 minutes | |
| 5-300 mg R-6-EAPB | minutes or earlier | after First Active Agent | |
| | | oral Tpeak | |
| Agent 1 | immediate release, for | delayed release, for | — |
| 5-300 mg S-2-EBPB | example an oral Tpeak | example an oral Tpeak | |
| Agent 2 | of about 45 to about 75 | of about 15 minutes | |
| 5-300 mg R-2-EBPB | minutes or earlier | after First Active Agent | |
| | | oral Tpeak | |
| Agent 1 | immediate release, for | delayed release, for | — |
| 5-300 mg S-5-EBPB | example an oral Tpeak | example an oral Tpeak | |
| Agent 2 | of about 45 to about 75 | of about 15 minutes | |
| 5-300 mg R-5-EBPB | minutes or earlier | after First Active Agent | |
| | | oral Tpeak | |
| Agent 1 | immediate release, for | delayed release, for | — |
| 5-300 mg S-6-EBPB | example an oral Tpeak | example an oral Tpeak | |
| Agent 2 | of about 45 to about 75 | of about 15 minutes | |
| 5-300 mg R-6-EBPB | minutes or earlier | after First Active Agent | |
| | | oral Tpeak | |

In certain embodiments Agent 1 is the first active agent with lower Tpeak as it is formulated for immediate release, and Agent 2 is the second active agent higher Tpeak as it is formulated for delayed release.

In certain alternate embodiments Agent 2 is the first active agent with lower Tpeak as it is formulated for immediate release, and Agent 1 is the second active agent higher Tpeak as it is formulated for delayed release.

### Synthetic Methods

Methods for synthesis of the compounds described herein and/or starting materials are either described in the art or will be readily apparent to the skilled artisan in view of general references well-known in the art (see, e.g., Green et al., “Protective Groups in Organic Chemistry,” (Wiley, 2nd ed. 1991); Harrison et al., “Compendium of Synthetic Organic Methods,” Vols. 1-8 (John Wiley and Sons, 1971-1996); “Beilstein Handbook of Organic Chemistry,” Beilstein Institute of Organic Chemistry, Frankfurt, Germany; Feiser et al, “Reagents for Organic Synthesis,” Volumes 1-17, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” John Wiley & Sons, 1995) and may be used to synthesize the compounds of the invention.

Additional references include: Taniguchi et al. 2010. Journal of mass spectrometry, 45(12), 1473-1476; Shulgin & Shulgin. 1992. PiHKAL. A chemical love story, Transform Press, Berkeley CA; Glennon et al. 1986. J. Med. Chem., 29(2), 194-199; Nichols et al. 1991. J. Med. Chem., 34(1), 276-281; Kedrowski et al. 2007. Organic Letters, 9(17), 3205-3207; Heravi & Zadsirjan. 2016. Current Organic Synthesis, 13(6), 780-833; Keri et al. 2017. European J. Med. Chem., 138, 1002-1033; Perez-Silanes et al. 2001. J. Heterocyclic Chem, 38(5), 1025-1030; and references therein.

### Synthesis 1. 5-MBPB

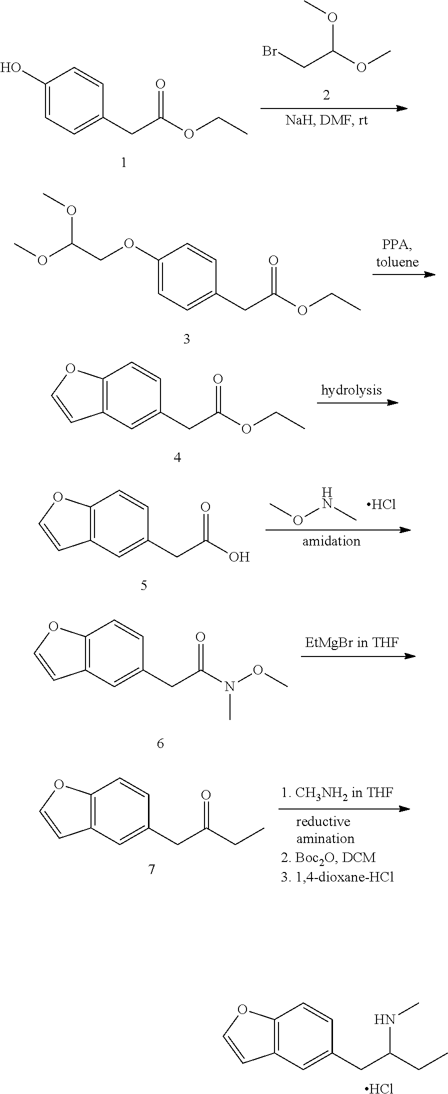

### Synthesis 2. 6-MBPB

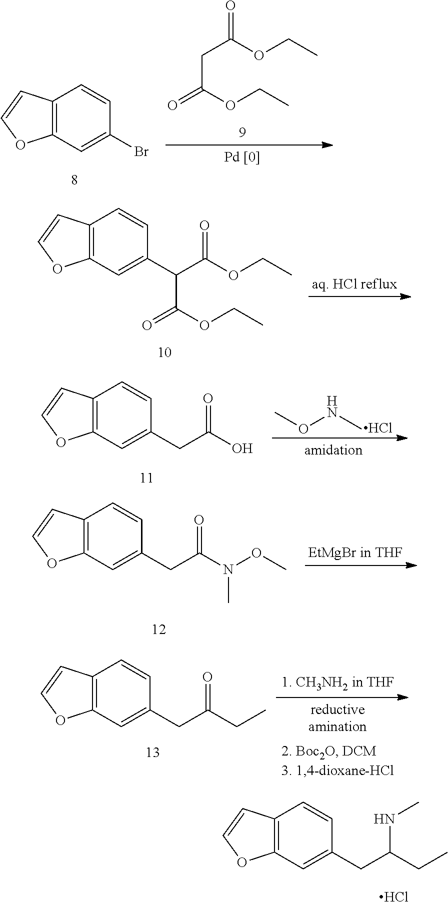

Other versions of these molecules can, for example, be synthesized following the methods of López and colleagues (López et al. 2012. British Journal of Pharmacology. 167 (2): 407-420). Additionally, the 5-MAPB and 6-MAPB can be made by analogy using the syntheses herein for 5-MBPB and 6-MBPB, using MeMgBr in THF in place of EtMgBr in THF in the third step.

### Synthesis 3. Bk-5-MAPB

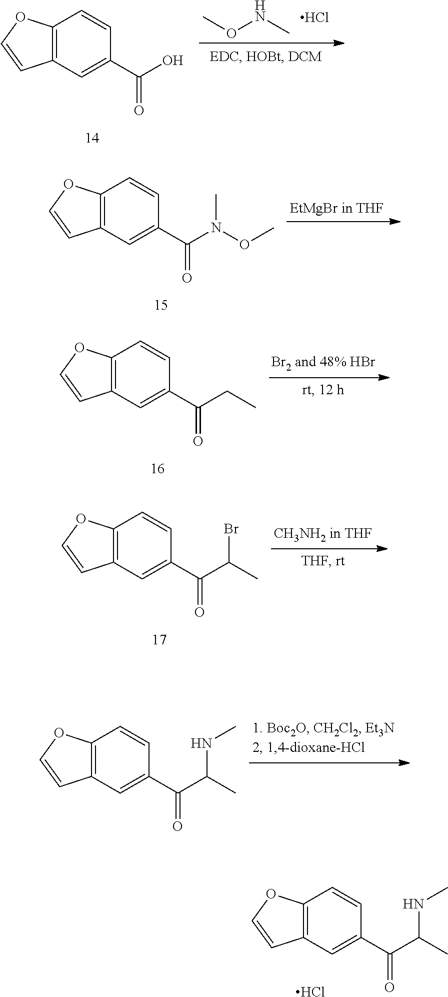

### Synthesis 4. Bk-5-MAPB

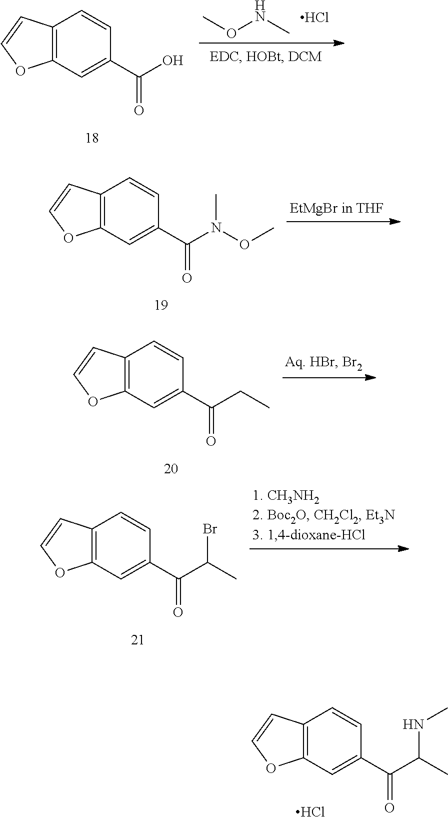

Other versions of these molecules can, for example, be synthesized following the methods of López and colleagues (López et al. 2012. British Journal of Pharmacology. 167 (2): 407-420). Additionally, the Bk-5-MBPB and Bk-66-MBPB can be made by analogy using the syntheses herein for Bk-5-MAPB and Bk-6-MAPB, using propyl magnesium bromide in THF in place of EtMgBr in THE in the second step.

### Synthesis 5. Alternative Method of Synthesis of Bk-5-MAPB and Bk-6-MAPB

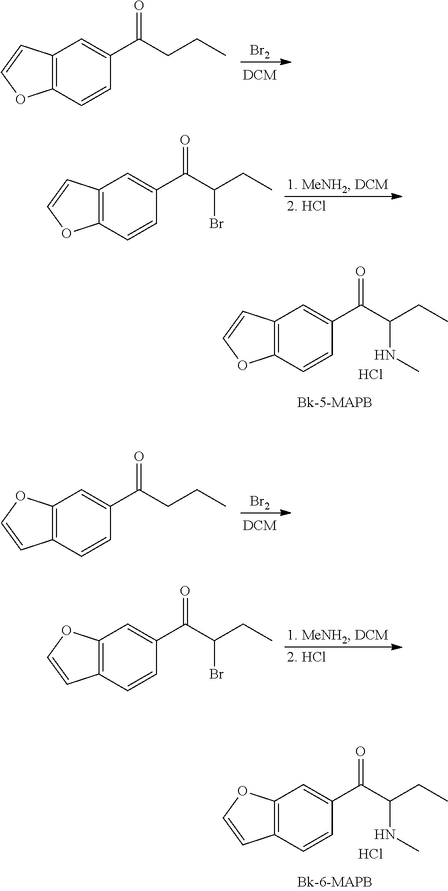

### Synthesis 6. Alternative Method of Synthesis of Bk-5-MAPB and Bk-6-MAPB

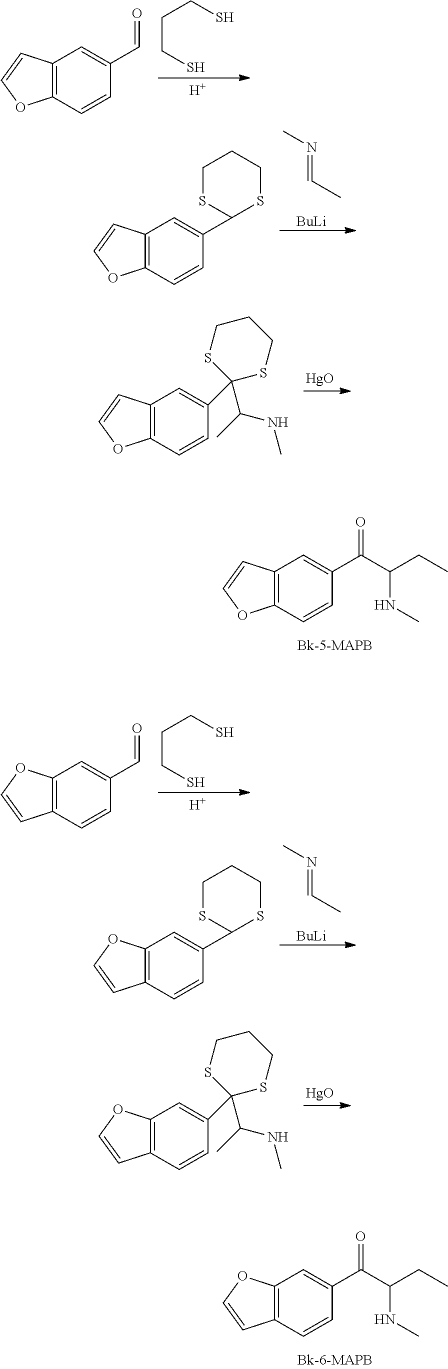

### Synthesis 7. Derivatization from Bk-5-MAPB

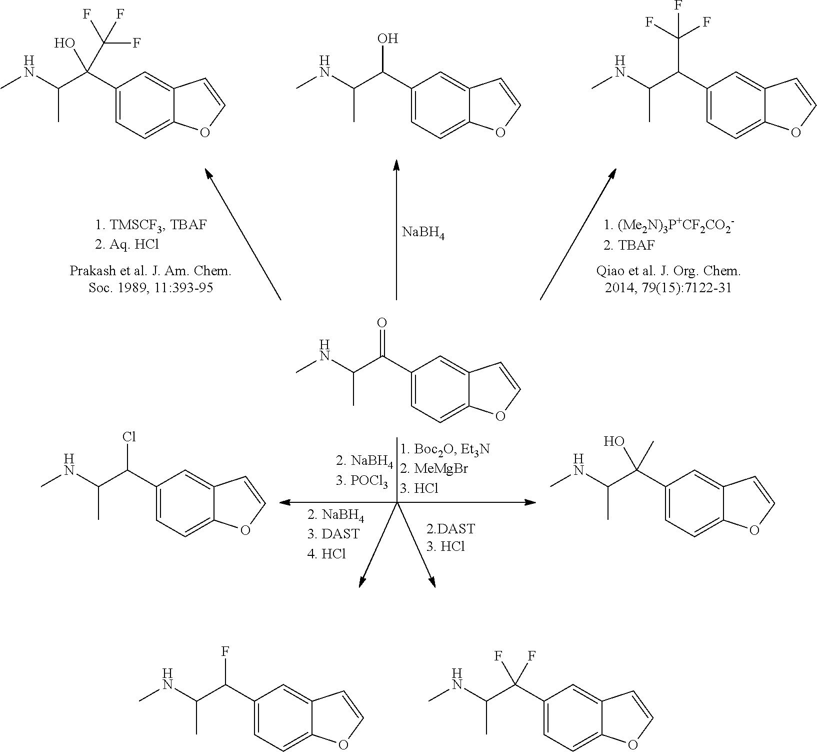

### Synthesis 8. Synthesis of 3-(benzofuran-6-yl)-N-methylbut-3-en-2-amine (Compound 1-4)

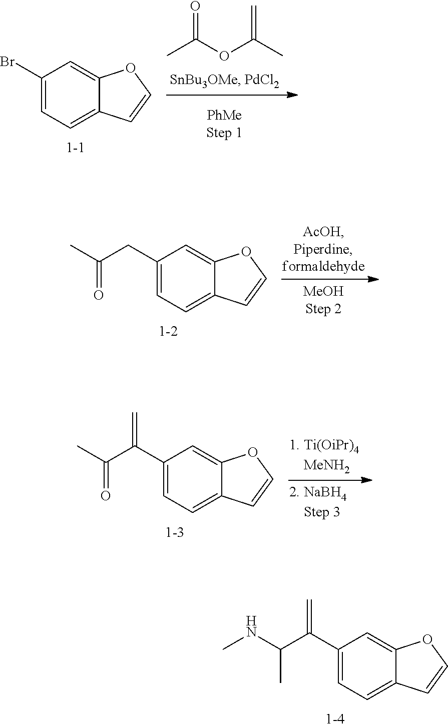

Step 1: A round-bottom flask is charged with 1-1, tributyltin methoxide, and palladium(II) chloride. The flask is then evacuated and refilled with anhydrous nitrogen three times before adding toluene and isopropenyl acetate. The reaction solution is then stirred with heating under nitrogen until the reaction is judged complete by TLC, HPLC, or other analytical method.

Following the reaction, the mixture is cooled to room temperature, diluted with ethyl acetate, and washed three times with water. The organic layer is then dried over anhydrous Na₂SO₄, filtered, and concentrated to collect crude 1-2. This crude material can be taken to the next step without further purification or purified by standard techniques of the art to obtain the pure compound.

Step 2: A round-bottom flask is charged with 1-2, acetic acid, piperdine, and formaldehyde. Methanol is then added to dissolve the reaction components and the mixture is stirred until the reaction is judged complete by TLC, HPLC, or other analytical method. Following the reaction, the mixture is diluted with ethyl acetate and washed three times with water. The organic layer is then dried over anhydrous Na₂SO₄, filtered, and concentrated to collect crude 1-3. This crude material can be taken to the next step without further purification or purified by standard techniques of the art to obtain the pure compound.

Step 3: In a round-bottom flask, 1-3, methylamine, and titanium (IV) isopropoxide are dissolved in ethanol and stirred under nitrogen. Once there is no remaining 1-3 as judged by TLC, HPLC, or other analytical method, the flask is opened briefly, and sodium borohydride is added slowly. The resulting slurry is stirred at room temperature overnight. Following the reaction, the mixture is diluted with ethyl acetate and washed three times with water. The organic layer is then dried over anhydrous Na₂SO₄, filtered, and concentrated to collect crude 1-4. This crude material can be purified by standard techniques of the art to obtain the pure compound.

The individual enantiomers of 1-4 can be separated using the methods described herein. For example, chiral SFC conditions are provided in Example 1. Following isolation of the pure enantiomers, they can be mixed again in any ratio necessary to obtain the desired effects.

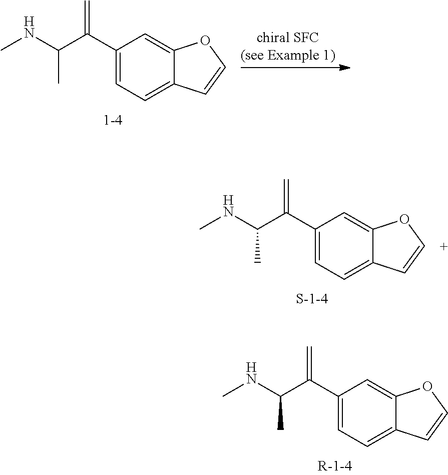

### Synthesis 9. Synthesis of 2-(benzofuran-6-yl)-3-(methylamino)butan-1-ol (Compound 2-6)

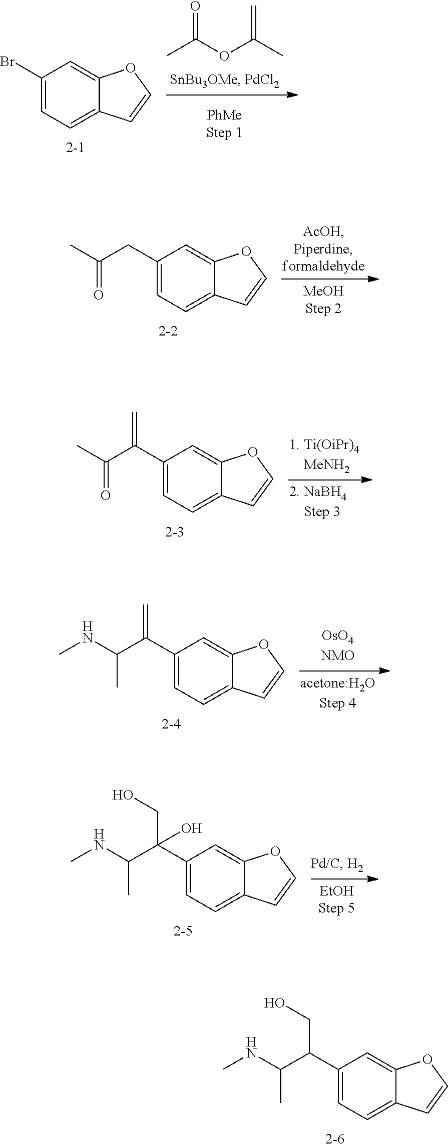

Step 1: A round-bottom flask is charged with 2-1, tributyltin methoxide, and palladium(II) chloride. The flask is then evacuated and refilled with anhydrous nitrogen three times before adding toluene and isopropenyl acetate. The reaction solution is then stirred with heating under nitrogen until the reaction is judged complete by TLC, HPLC, or other analytical method. Following the reaction, the mixture is cooled to room temperature, diluted with ethyl acetate, and washed three times with water. The organic layer is then dried over anhydrous Na₂SO₄, filtered, and concentrated to collect crude 2-2. This crude material can be taken to the next step without further purification or purified by standard techniques of the art to obtain the pure compound.

Step 2: A round-bottom flask is charged with 2-2, acetic acid, piperdine, and formaldehyde. Methanol is then added to dissolve the reaction components and the mixture is stirred until the reaction is judged complete by TLC, HPLC, or other analytical method. Following the reaction, the mixture is diluted with ethyl acetate and washed three times with water. The organic layer is then dried over anhydrous Na₂SO₄, filtered, and concentrated to collect crude 2-3. This crude material can be taken to the next step without further purification or purified by standard techniques of the art to obtain the pure compound.

Step 3: In a round-bottom flask, 2-3, methylamine, and titanium (IV) isopropoxide are dissolved in ethanol and stirred under nitrogen. Once there is no remaining 2-3 as judged by TLC, HPLC, or other analytical method, the flask is opened briefly, and sodium borohydride is added slowly. The resulting slurry is stirred at room temperature overnight. Following the reaction, the mixture is diluted with ethyl acetate and washed three times with water. The organic layer is then dried over anhydrous Na₂SO₄, filtered, and concentrated to collect crude 2-4. This crude material can be purified by standard techniques of the art to obtain the pure compound.

Step 4: To a round-bottom flask containing 2-4 dissolved in acetone:H₂O is added NMO and a catalytic amount of osmium tetroxide. The resulting mixture is stirred at room temperature until the reaction is judged complete by TLC, HPLC, or other analytical method. Following the reaction, the mixture is diluted with ethyl acetate and washed three times with water. The organic layer is then dried over anhydrous Na₂SO₄, filtered, and concentrated to collect crude 2-5. This crude material can be taken to the next step without further purification or purified by standard techniques of the art to obtain the pure compound.

Step 5: A round-bottom flask containing 2-5 and palladium on carbon is evacuated under vacuum and backfilled with nitrogen three times. Ethanol is then added to the flask and the resulting mixture is sparged with hydrogen gas while stirring. Once the nitrogen atmosphere is displaced by hydrogen, the reaction is stirred at room temperature until the reaction is judged complete by TLC, HPLC, or other analytical method. Following the reaction, the mixture is diluted with ethyl acetate, filtered through diatomaceous earth, and concentrated to collect crude 2-6. This crude material can be purified by standard techniques of the art to obtain the pure compound.

The individual enantiomers of 2-6 can be separated using the methods described herein. For example, chiral SFC conditions are provided in Example 1. Following isolation of the pure enantiomers, they can be mixed again in any ratio necessary to obtain the desired effects.

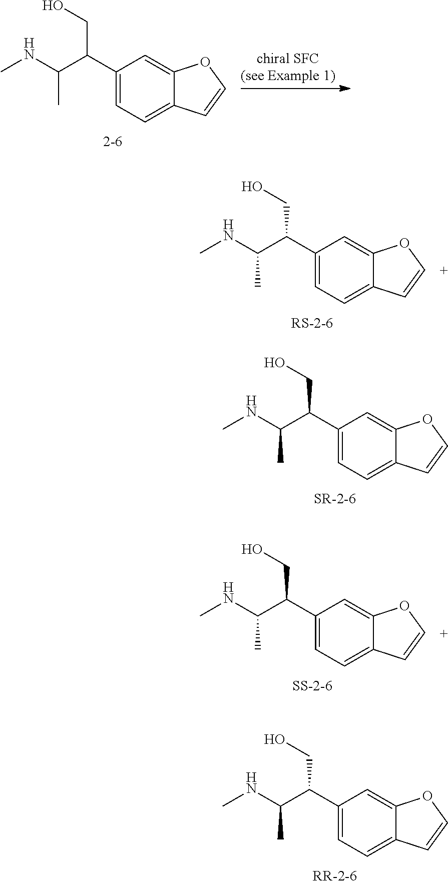 [SMILES: CNC(C)C(CO)c1ccc2ccoc2c1 ; CN[C@@H](C)[C@H](CO)c1ccc2ccoc2c1 ; CN[C@H](C)[C@@H](CO)c1ccc2ccoc2c1 ; CN[C@@H](C)[C@@H](CO)c1ccc2ccoc2c1 ; CN[C@H](C)[C@H](CO)c1ccc2ccoc2c1]

Alternatively, the diastereomers can first be separated by conventional, achiral purification techniques such as silica gel chromatography or preparative HPLC. The two purified diastereomers can then be further separated into the enantiomers as described.

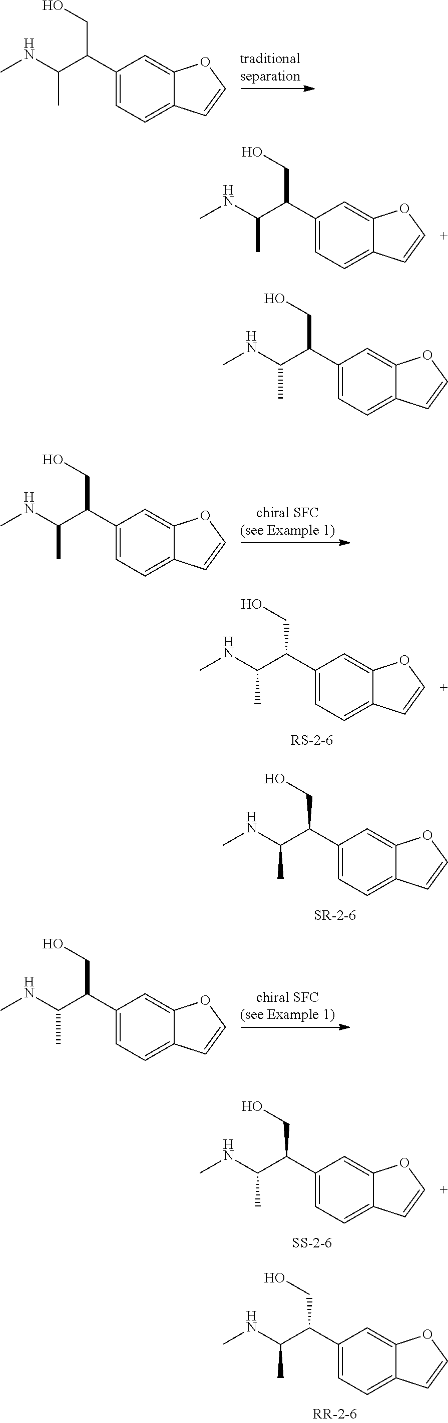

### Synthesis 10. Synthesis of 2-(benzofuran-6-yl)-1-cyclopropyl-N-ethylethan-1-amine (Compound 3-5)

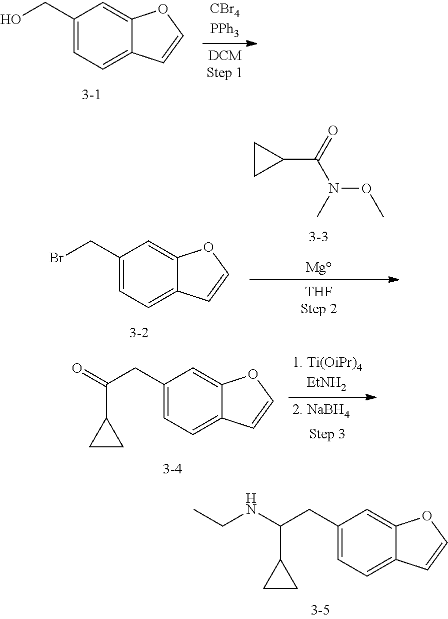

Step 1: To a round-bottom flask containing 3-1 dissolved in DCM is added triphenylphosphine and tetrabromomethane. The resulting mixture is stirred at room temperature until the reaction is judged complete by TLC, HPLC, or other analytical method. Following the reaction, the mixture is diluted with ethyl acetate and washed three times with water. The organic layer is then dried over anhydrous Na₂SO₄, filtered, and concentrated to collect crude 3-2. This crude material can be purified by standard techniques of the art to obtain the pure compound.

Step 2: A round-bottom flask is charged with freshly activated magnesium metal then evacuated under reduced pressure and back-filled with nitrogen three times. Anhydrous THE is then added, and the reaction solution cooled to −78° C. followed by the slow addition of 3-2. Once reaction mixture ceases to self-heat, an anhydrous solution of 3-3 is added slowly. The resulting mixture is allowed to gradually warm to room temperature overnight. The reaction is then quenched under nitrogen using a saturated solution of aqueous NH₄Cl. The resulting mixture is then diluted with EtOAc, washed three times with water, dried over anhydrous Na₂SO₄, and filtered. The filtrate is then concentrated to collect crude 3-4. This crude material can be taken to the next step without further purification or purified by standard techniques of the art to obtain the pure compound.

Step 3: In a round-bottom flask, 3-4, ethylamine, and titanium (IV) isopropoxide are dissolved in ethanol and stirred under nitrogen. Once there is no remaining 3-4 as judged by TLC, HPLC, or other analytical method, the flask is opened briefly, and sodium borohydride is added slowly. The resulting slurry is stirred at room temperature overnight. Following the reaction, the mixture is diluted with ethyl acetate and washed three times with water. The organic layer is then dried over anhydrous Na₂SO₄, filtered, and concentrated to collect crude 3-5. This crude material can be purified by standard techniques of the art to obtain the pure compound.

The individual enantiomers of 3-5 can be separated using the methods described herein. For example, chiral SFC conditions are provided in Example 1. Following isolation of the pure enantiomers, they can be mixed again in any ratio necessary to obtain the desired effects.

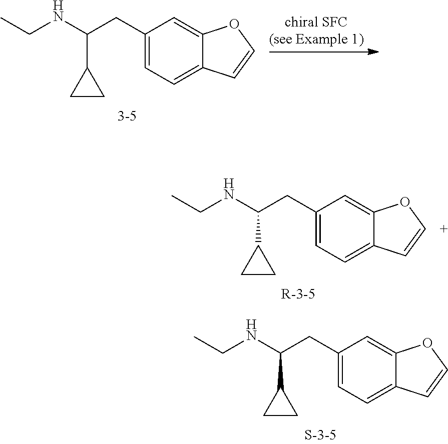 [SMILES: CCNC(Cc1ccc2ccoc2c1)C1CC1 ; CCN[C@H](Cc1ccc2ccoc2c1)C1CC1 ; CCN[C@@H](Cc1ccc2ccoc2c1)C1CC1]

### Synthesis 11. Synthesis of 3-(benzofuran-6-yl)-4-fluoro-2-(methylamino)butane-1,3-diol (Compound 4-8)

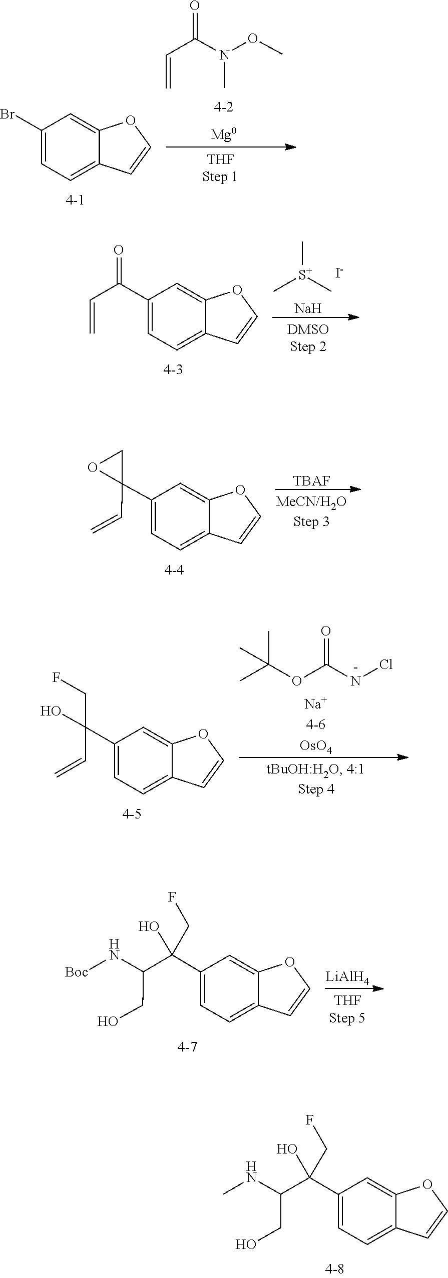

Step 1: A round-bottom flask is charged with freshly activated magnesium metal then evacuated under reduced pressure and back-filled with nitrogen three times. Anhydrous THE is then added, and the reaction solution cooled to −78° C. followed by the slow addition of 4-1. Once the reaction mixture ceases to self-heat, an anhydrous solution of 4-2 is added slowly. The resulting mixture is allowed to gradually warm to room temperature overnight. The reaction is then quenched under nitrogen using a saturated solution of aqueous NH₄Cl. The resulting mixture is then diluted with EtOAc, washed three times with water, dried over anhydrous Na₂SO₄, and filtered. The filtrate is then concentrated to collect crude 4-3. This crude material can be taken to the next step without further purification or purified by standard techniques of the art to obtain the pure compound.

Step 2: A round-bottom flask is charged with a stirbar, anhydrous DMSO, and trimethylsulfonium iodide. After evacuating the flask of ambient air and refilling with dry nitrogen three times, NaH is added slowly to the flask. Once the reaction solution has stopped giving off hydrogen gas, an anhydrous solution of 4-3 in DMSO is added slowly. The reaction is allowed to stir overnight and warm to room temperature. The reaction is then quenched under nitrogen using a saturated solution of aqueous NH₄Cl. The resulting mixture is then diluted with EtOAc, washed three times with water, dried over anhydrous Na₂SO₄, and filtered. The filtrate is then concentrated to collect crude 4-4. This crude material can be taken to the next step without further purification or purified by standard techniques of the art to obtain the pure compound.

Step 3: A round-bottom flask is charged with a stirbar, 4-4, and TBAF. The reagents are then dissolved in a solution of MeCN/H₂O, heated to just below reflux temperature, and stirred overnight. The reaction is monitored until completion by TLC, HPLC, or other analytical method. Following the reaction, the mixture is diluted with ethyl acetate and washed three times with water. The organic layer is then dried over anhydrous Na₂SO₄, filtered, and concentrated to collect crude 3-5. This crude material can be purified by standard techniques of the art to obtain the pure compound.

Step 4: A round-bottom flask is charged with a stirbar, 4-6, osmium tetroxide, and 4-5. The reagents are then dissolved in a solution of 4:1 tBuOH:H₂O. The resulting mixture is stirred at room temperature until the reaction is judged complete by TLC, HPLC, or other analytical method. Following the reaction, the mixture is diluted with ethyl acetate and washed three times with water. The organic layer is then dried over anhydrous Na₂SO₄, filtered, and concentrated to collect crude a mixture of regio- and diastereoisomers of 4-7. This crude material can be purified by standard techniques of the art to obtain the pure compound.

Step 5: To a flame-dried round-bottom flask is added a stirbar, 4-7, and anhydrous THF. The resulting solution is cooled to −78° C. before adding LiAlH₄ slowly via syringe. The resulting mixture is allowed to slowly warm to room temperature and stirred until the reaction is judged complete by TLC, HPLC, or other analytical method. Following the reaction, the mixture is diluted with ether, slowly quenched with aqueous NaOH, then further quenched with water. The resulting slurry is diluted with EtOAc and washed three times with water. The organic layer is then dried over anhydrous Na₂SO₄, filtered, and concentrated to crude 4-8. This crude material can be purified by standard techniques of the art to obtain the pure compound.

The individual enantiomers of 4-8 can be separated using the methods described herein. For example, chiral SFC conditions are provided in Example 1. Following isolation of the pure enantiomers, they can be mixed again in any ratio necessary to obtain the desired effects.

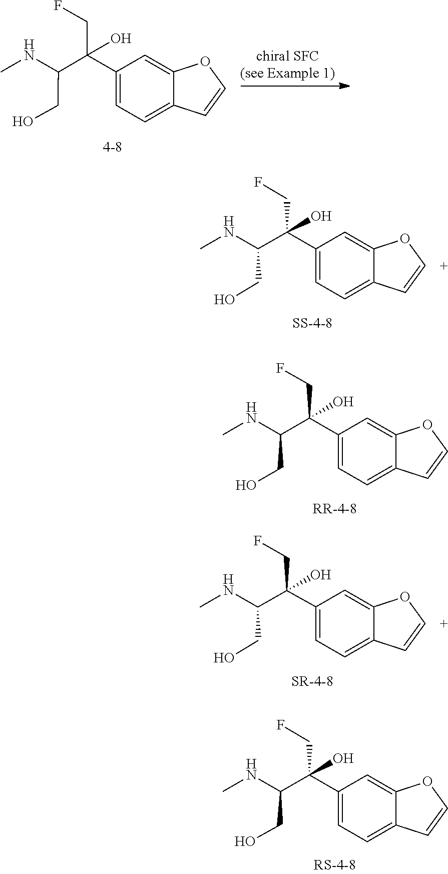 [SMILES: CNC(CO)C(O)(CF)c1ccc2ccoc2c1 ; CN[C@@H](CO)[C@](O)(CF)c1ccc2ccoc2c1 ; CN[C@H](CO)[C@@](O)(CF)c1ccc2ccoc2c1 ; CN[C@@H](CO)[C@@](O)(CF)c1ccc2ccoc2c1 ; CN[C@H](CO)[C@](O)(CF)c1ccc2ccoc2c1]

Alternatively, the diastereomers can first be separated by conventional, achiral purification techniques such as silica gel chromatography or preparative HPLC. The two purified diastereomers can then be further separated into the enantiomers as described.

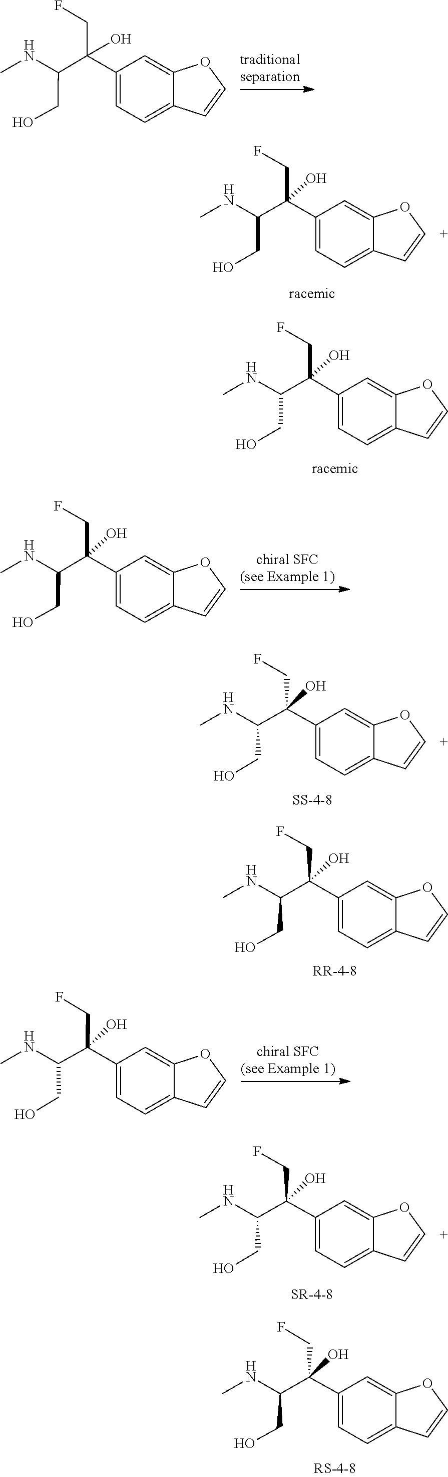

### Synthesis 12. Synthesis of 1-(benzofuran-5-yl)-N-methylpropan-2-amine (5-MAPB)

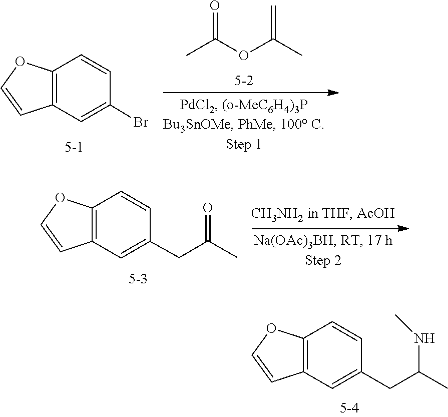

Step 1: To a stirred solution of 5-bromobenzofuran (5-1) (20 g, 101.52 mmol, 1 eq.) in dry toluene (400 mL) was added tri(o-tolyl)phosphine (1.84 g, 6.09 mmol, 0.06 eq.), tributyl tin methoxide (48.89 mL, 152.28 mmol, 1.5 eq.) and Isopropenyl acetate (16.99 mL, 156.34 mmol, 1.54 eq.) then the resulting reaction mixture was degassed under nitrogen for 15 minutes. Then palladium (II) chloride (1.26 g, 7.10 mmol, 0.07 eq.) was added to the reaction mixture and the resulting reaction mixture was heated to 100° C. for 16 h. Upon completion, monitored by TLC (10% EA in Hexane), the reaction mixture was cooled to RT, evaporate under vacuum. Then the residue was dissolved in ethyl acetate and filtered through celite bed, washed with water, and saturated potassium fluoride solution, followed by brine solution. Combined organic layer was dried over anhydrous sodium sulphate, solvent was removed under vacuum and purified by silica gel column chromatography using ethyl acetate/hexane (10:90 v/v) as eluent to afford 1-(benzofuran-5-yl)propan-2-one (5-3) as light yellow gum (17 g, 96%).¹H NMR (400 MHz, DMSO-d₆) δ 7.96 (d, J=2.0 Hz, 1H), 7.53 (d, J=8.48 Hz, 1H), 7.46 (s, 1H), 7.13 (dd, J=1.52 Hz, 8.44 Hz, 1H), 6.92 (bs, 1H), 3.83 (s, 2H), 2.12 (s, 3H). LCMS: (ES) C₁₁H₁₀O₂ requires 174, found 175 [M+H]⁺.

Step 2: To a stirred solution of 1-(benzofuran-5-yl)propan-2-one (5-3) (16.0 g, 91.84 mmol, 1.0 eq.) in AcOH (70 ml) was added Methyl Amine (2M in THF) (230 mL, 460 mmol, 5 eq.) at RT and the resulting reaction mixture was stirred at RT for 1 h. Then Na(OAc)₃BH (29.2 g, 137.77 mmol, 1.5 eq.) was added portion wise to the reaction mixture and continue to stir at RT for 16 h. After completion of reaction (TLC and LCMS) the reaction mixture was diluted with water (100 mL), and extracted with DCM (50 mL×2). Combined organic layer was dried over anhydrous sodium sulphate, solvent was removed under vacuum to got crude 1-(benzofuran-5-yl)-N-methylpropan-2-amine (5-MAPB) (16.0 g, 92%). 1H NMR (400 MHz, DMSO-d₆) δ 7.93 (s, 1H), 7.49 (d, J=8.36 Hz, 1H), 7.43 (s, 1H), 7.12 (d, J=7.56 Hz, 1H), 6.88 (s, 1H), 2.84-2.79 (m, 1H), 2.74-2.69 (m, 1H), 2.49 (bs, 1H), 2.94 (s, 3H), 0.91 (d, J=6.08 Hz, 3H). LCMS: (ES) C12H15NO requires 189, found 190 [M+H]⁺.

### Synthesis 13. Synthesis of 1-(benzofuran-6-yl)-N-methylpropan-2-amine (6-MAPB)

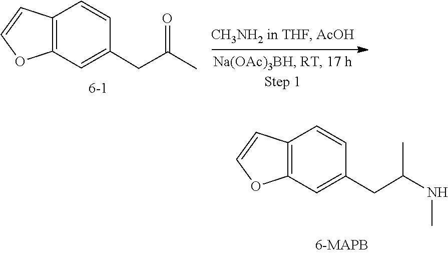

Step 1: To a stirred solution of 1-(benzofuran-6-yl)propan-2-one (6-1) (7 g, 40.23 mmol) in ACOH (15 mL), methyl amine (100 mL, 2M in methanol, 200 mmol) was added to it. After stirring for 15 mins, Na(OAc)₃BH (12.7 g, 60.34 mmol) was added to the reaction mixture and continue to stir at room temperature for 17 h. After the completion [Monitored with TLC, Mobile Phase 10% MeOH-DCM], the excess solvent was evaporated under reduced pressure and basified by sodium carbonate solution (30 mL) and extracted with DCM (2×50 mL). The obtained crude 1-(benzofuran-6-yl)-N-methylpropan-2-amine (6-MAPB) (7 g) was forwarded to the next step without further purification. ¹H NMR (400 MHz, DMSO-d₆): δ 7.90 (d, J=1.92 Hz, 1H), 7.54 (d, J=7.88 Hz, 1H), 7.39 (s, 1H), 7.08 (d, J=7.68 Hz, 1H), 6.89 (s, 1H), 2.85-2.80 (m, 1H), 2.74-2.65 (m, 2H), 2.28 (s, 3H), 0.91-0.85 (m, 3H). LCMS: (ES) C12H15NO requires 189.12, found 190.07 [M+H]⁺.

### Synthesis 14. Synthesis of 1-(benzofuran-5-yl)-N-methylbutan-2-amine (5-MBPB)

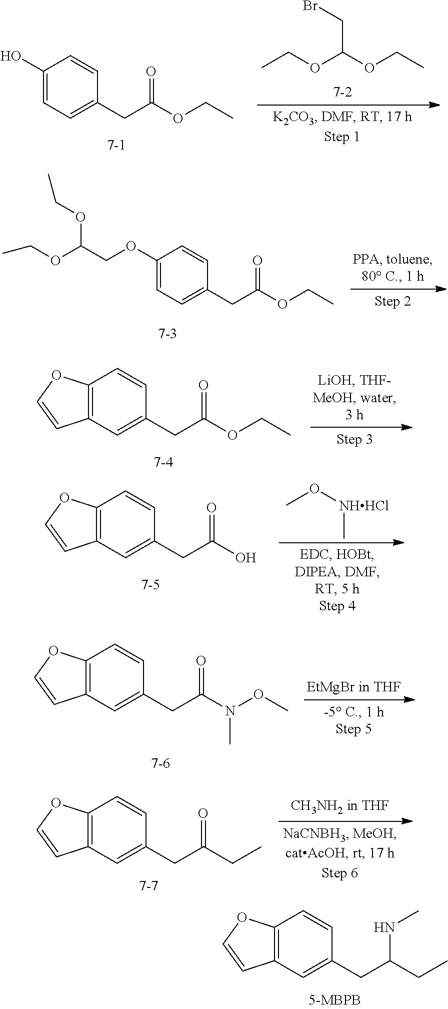

Step 1: To a stirred solution of ethyl 2-(4-hydroxyphenyl)acetate (7-1) (40 g, 222.22 mmol, 1.0 eq.) and 2-bromo-1,1-diethoxyethane (36.76 mL, 244.4 mmol, 1.1 eq.) in DMF (250 mL) was added K₂CO₃ (92 g, 666.66 mmol, 3.0 eq.) and heated to 100° C. for 17 h. After the completion [Monitored by TLC, mobile phase 10% EtOAc-Hexane], mixture was quenched with ice cold water (500 mL) and extracted with 30% ethyl acetate in hexane (1 L). Then the organic part was washed with saturated solution of NaCl, dried over anhydrous magnesium sulphate and concentrated under vacuum to afford the crude which was purified by silica gel (100-200 mesh) column chromatography eluted with 0-10% ethyl acetate in hexane to get the desired compound ethyl 2-(4-(2,2-diethoxyethoxy)phenyl)acetate (7-3) (20 g, 30%) as a colorless liquid. ¹H NMR (400 MHz, DMSO-d₆): δ 7.17 (d, J=8.56 Hz, 2H), 6.90 (d, J=8.52 Hz, 2H), 4.78 (t, J=5.2 Hz, 1H), 4.08 (m, 2H), 3.93 (d, J=5.2 Hz, 2H), 3.70-3.44 (m, 6H), 1.18-1.08 (m, 9H).

Step 2: To a stirred solution of ethyl 2-(4-(2,2-diethoxyethoxy)phenyl)acetate (7-3) (20 g, 74.62 mmol, 1.0 eq.) in toluene (100 mL) was added PPA (21.94 g, 223.8 mmol, 3.0 eq.) and heated to 80° C. for 3 h under nitrogen atmosphere. After the completion [Monitored with TLC, mobile phase 10% EtOAc-Hexane], reaction mixture was quenched with ice cold water (100 mL) and extracted with 30% ethyl acetate in hexane (300 mL). Then the organic part washed with saturated solution of NaCl, dried over anhydrous magnesium sulphate and concentrated under vacuum to afford the crude which was purified by silica gel (100-200 mesh) column chromatography eluted with 0-2% ethyl acetate in hexane to get the desired ethyl 2-(benzofuran-5-yl)acetate (7-4) (4.0 g, 26%) as a colorless liquid. ¹H NMR (400 MHz, DMSO-d₆): δ 7.97 (d, J=2.08 Hz, 1H), 7.54 (d, J=8.44 Hz, 2H), 7.20 (t, J=1.36 Hz, J=8.48 Hz, 1H), 6.93 (d, J=1.92 Hz, 1H), 4.10-4.04 (m, 2H), 3.73 (s, 2H), 1.17 (t, J=7 Hz, J=7.2 Hz, 3H).

Step 3: To a stirred solution of ethyl 2-(benzofuran-5-yl)acetate (7-4) (4 g, 19.6 mmol, 1.0 eq.) in THE (20 mL), MeOH (20 mL) was added followed by addition of lithium hydroxide (1.4 g, 58.82 mmol, 3.0 eq.) in water (20 mL). Reaction was stirred at RT for 2 hrs. After the completion [Monitored with TLC, Mobile Phase 60% EtOAc-Hexane], excess solvent was evaporated and acidified with 1(N) HCL in ice cooling condition and extracted with 10% MeOH in DCM. Organic part was washed with saturated solution of NaCl, dried over anhydrous magnesium sulphate and concentrated under vacuum to afford 2-(benzofuran-5-yl)acetic acid (7-5) (3.3 g, 95%) as an off white solid. ¹H NMR (400 MHz, DMSO-d₆): δ 12.28 (s, 1H), 7.96 (d, J=2.0 Hz, 1H), 7.52 (d, J=8.68 Hz, 2H), 7.20-7.18 (m, 1H), 6.92 (bs, 1H), 3.64 (s, 2H).

Step 4: To a stirred solution of 2-(benzofuran-5-yl)acetic acid (7-5) (3.3 g, 18.75 mmol, 1.0 eq.) in DMF (20 mL) were added DIPEA (9.8 mL, 56.25 mmol, 3.0 eq.), EDCI (3.93 g, 20.62 mmol, 1.1 eq.) and HOBT (3.79 g, 28.12 mmol, 1.5 eq.). Reaction was stirred at RT for 5 min followed by addition of weinreb amide (2 g, 20.62 mmol, 1.1 eq.). Reaction was stirred at RT for overnight. After the completion [Monitored with TLC, Mobile Phase 30% EtOAc-Hexane], reaction mixture was diluted with ethyl acetate (200 mL), washed 2-3 times with cold water. Organic phase was dried over magnesium sulphate and concentrated under reduced pressure to afford 2-(benzofuran-5-yl)-N-methoxy-N-methylacetamide (7-6) (4 g, 97%) as a light yellow sticky solid. ¹H NMR (400 MHz, DMSO-d₆): δ 7.95 (d, J=2.08 Hz, 1H), 7.51-7.49 (m, 2H), 7.18 (dd, J=1.36 Hz, 8.6 Hz, 1H), 6.91 (d, J=1.8 Hz, 1H), 3.80 (s, 2H), 3.67 (s, 3H), 3.11 (s, 3H).

Step 5: To a stirred solution of 2-(benzofuran-5-yl)-N-methoxy-N-methylacetamide (7-6) (4 g, 18.26 mmol, 1.0 eq.) in THE (20 mL), ethyl magnesium bromide (1 M, 27.39 mL, 27.39 mmol, 1.5 eq.) was added drop wise at 0° C. under nitrogen atmosphere. The reaction mixture was stirred at 0° C. for 1 hr. After completion [Monitored with TLC, mobile Phase 10% EtOAc-Hexane], it was quenched by saturated ammonium chloride solution (5 mL) and extracted with ethyl acetate (50 mL) and washed with NaCl solution. Organic phase was dried over magnesium sulphate and concentrated under reduced pressure. Crude compound was purified by silica gel (100-200 mesh) column chromatography eluted with 10-20% ethyl acetate in hexane to afford the desired 1-(benzofuran-5-yl)butan-2-one (7-7) (3.2 g, 93%) as a yellow liquid. ¹H NMR (400 MHz, DMSO-d₆): δ 7.96 (d, J=1.92 Hz, 1H), 7.52 (d, J=8.4 Hz, 1H), 7.46 (s, 1H), 7.12 (d, J=7.36 Hz, 1H), 6.91 (bs, 1H), 3.82 (s, 2H), 2.53 (m, 2H), 0.91 (t, J=7.24 Hz, J=7.28 Hz, 3H).

Step 6: To a stirred solution of 1-(benzofuran-5-yl)butan-2-one (7-7) (3.2 g, 17.02 mmol, 1.0 eq) and methanol (20 mL), methyl amine (43 mL, 2M in methanol, 85.1 mmol, 5.0 eq) was added followed by addition of catalytic amount of AcOH (0.5 mL). After stirring for 15 mins, NaCNBH3 (3.2 g, 51.06 mmol, 3.0 eq) was added. The resultant mixture was stirred at room temperature for 17 h. After the completion [Monitored with TLC, Mobile Phase 5% MeOH-EtOAc, Rf-0.2], the excess solvent was evaporated under reduced pressure and basified by sodium carbonate solution (30 mL) and extracted with DCM (2×100 mL). The obtained crude 1-(benzofuran-5-yl)-N-methylbutan-2-amine (5-MBPB) (3.3 g, 95%). ¹H NMR (400 MHz, DMSO-d₆): δ 7.94-7.91 (m, 1H), 7.50-7.46 (m, 2H), 7.15 (d, J=8.4 Hz, 1H), 6.89 (d, J=1.72 Hz, 1H), 2.82-2.61 (m, 3H), 2.32 (s, 3H), 1.40-1.30 (m, 2H), 0.95-0.75 (m, 3H). LCMS: (ES) C13H17NO requires 203, found 204 [M+H]⁺.

### Synthesis 15. Synthesis of 1-(benzofuran-6-yl)-N-methylbutan-2-amine (6-MBPB)

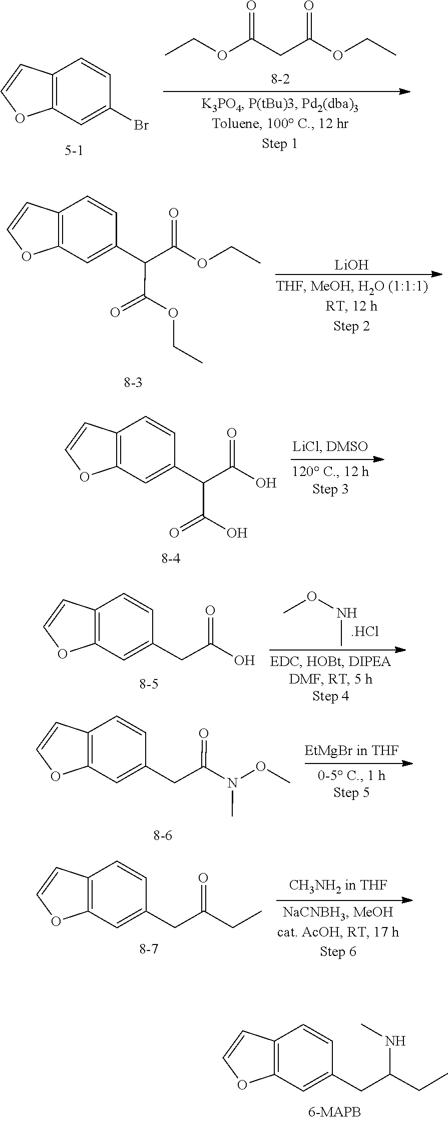

Step 1: A solution of diethyl malonate (8-2) (20.42 mL, 134.01 mmol, 1.1 eq.) and K₃PO ₄(51.65 g, 243.65 mmol, 2 eq.) in toluene (120 mL) was purged with nitrogen for 10 min. Then P(tBu)₃ (12.45 g, 24.36 mmol, 0.2 eq.) was added to the reaction mixture followed by 6-bromobenzofuran (8-1) (24 g, 121.82 mmol, 1.0 eq.) and Pd₂(dba)₃ (2.31 g, 2.43 mmol, 0.02 eq.). Reaction mixture was stir at RT and continue at 100° C. for 12 h. After completion of reaction monitored by TLC and LCMS, the mixture was cooled to room temperature and concentrated under reduced pressure. Then the reaction mixture was diluted with water [500 mL] and extracted with EtOAc [500 mL×2]. Organic layer was separated, dried over sodium sulphate and concentrated under vacuum. Then the crude was purified by silica gel (100-200 mesh) column chromatography eluted with 0-10% ethyl acetate in hexane to afford diethyl 2-(benzofuran-6-yl)malonate (8-3) (15 g, 44%) as a colorless liquid. ¹H NMR (400 MHz, DMSO-d₆): δ 8.01 (d, J=2.12 Hz, 1H), 7.63 (t, J=8.04 Hz, J=7.44 Hz, 2H), 7.28-7.26 (m, 1H), 6.96 (bs, 1H), 5.07 (s, 1H), 4.21-4.08 (m, 4H), 1.20-1.15 (m, 6H). LCMS: (ES) C15H16O5 requires 276, found 277 [M+H]+.

Step 2: To a stirred solution of diethyl 2-(benzofuran-6-yl)malonate (8-3) (15 g, 54.34 mmol, 1.0 eq.) in THF (50 mL), MeOH (50 mL) was added followed by addition of lithium hydroxide (5.7 g, 135.87 mmol, 2.5 eq.) in water (50 mL). Then the reaction was stir at RT for 12 h. After the completion [Monitored by TLC, mobile Phase 5% MeOH-DCM], excess solvent was evaporated and acidified with 1(N) HCL in ice cooling condition and extracted with 10% MeOH in DCM. Organic part was washed with saturated solution of NaCl, dried over anhydrous magnesium sulphate and concentrated under vacuum to afford 2-(benzofuran-6-yl)malonic acid (8-4) (11.5 g, 96%) as an off white solid. ¹H NMR (400 MHz, DMSO-d₆): δ 12.71 (s, 2H), 7.99 (d, J=2.08 Hz, 1H), 7.62-7.58 (m, 2H), 7.29 (d, J=14.68 Hz, 1H), 6.95 (d, J=1.84 Hz, 1H). LCMS: (ES) C11H805 requires 220, found 219 [M−H]+.

Step 3: To a stirred solution of 2-(benzofuran-6-yl)malonic acid (8-4) (11.5 g, 52.27 mmol, 1.0 eq) in DMSO (50 mL) were added LiCl (4.39 g, 104.54 mmol, 2.0 eq) and H₂O (5 mL) heated to 120° C. temperature for 12 hrs. After completion [Monitored with TLC, Mobile Phase 100% EtOAc, Rf-0.6], reaction mixture was diluted with water [250 mL] and extracted with EtOAc [500 mL×2]. Then the organic layer was extracted and dried over magnesium sulphate and concentrated under vacuum to afford 2-(benzofuran-6-yl)acetic acid (8-5) (9 g, 97.73%) as an off white solid crude. ¹H NMR (400 MHz, DMSO-d₆): δ 12.03 (s, 1H), 7.95 (d, J=2.0 Hz, 1H), 7.58 (d, J=7.92 Hz, 1H), 7.48 (s, 1H), 7.16 (d, J=7.88 Hz, 1H), 6.92 (d, J=0.92 Hz, 1H), 3.68 (s, 2H).

Step 4: To a stirred solution of 2-(benzofuran-6-yl)acetic acid (8-5) (9.0 g, 51.13 mmol, 1.0 eq.) in DMF (15 mL) were added DIPEA (26.74 mL, 153.40 mmol, 3.0 eq.), EDCI (10.74 g, 56.25 mmol, 1.1 eq.) and HOBT (8.62 g, 63.92 mmol, 1.5 eq.). The reaction mixture was stirred at RT for 5 min followed by addition of weinreb amide (5.45 g, 56.25 mmol, 1.1 eq.), then it was stir at RT for 5 h. After the completion [monitored by TLC, mobile Phase 30% EtOAc-hexane], reaction mixture was diluted with ethyl actate (500 mL), washed 2-3 times with cold water and dried over magnesium sulphate and concentrated under reduced pressure to afford 2-(benzofuran-6-yl)-N-methoxy-N-methylacetamide (8-6) (8.0 g, 71%) as a light yellow sticky solid. ¹H NMR (400 MHz, DMSO-d₆): δ 7.94 (d, J=2.04 Hz, 1H), 7.57 (d, J=7.92 Hz, 1H), 7.45 (s, 1H), 7.13 (d, J=7.96 Hz, 1H), 6.91 (bs, 1H), 3.83 (s, 2H), 3.68 (s, 3H), 3.11 (s, 3H). LCMS: (ES) C12H13NO3 requires 219, found 220 [M+H]⁺.

Step 5: To a stirred solution of 2-(benzofuran-6-yl)-N-methoxy-N-methylacetamide (8-6) (8.0 g, 36.53 mmol, 1.0 eq.) in THE (50 mL), ethyl magnesium bromide (1 M, 54.79 mL, 54.79 mmol, 1.5 eq.) was added drop wise at 0° C. under nitrogen atmosphere. The reaction mixture was stirred at 0° C. for 1 h. After completion [monitored by TLC, mobile Phase 10% EtOAc-hexane], it was quenched by saturated ammonium chloride solution (5 mL) and extracted with ethyl acetate (100 mL) and washed with NaCl solution then dried over magnesium sulphate and concentrated under reduced pressure. The crude compound was purified by silica gel (100-200 mesh) column chromatography eluted with 10-20% ethyl acetate in hexane to afford 1-(benzofuran-6-yl)butan-2-one (8-7) (6.0 g, 87%) as a yellow liquid. ¹H NMR (400 MHz, DMSO-d₆): δ 7.94 (d, J=2.16 Hz, 1H), 7.58 (d, J=7.92 Hz, 1H), 7.42 (s, 1H), 7.08 (d, J=8.0 Hz, 1H), 6.92 (t, J=0.76 Hz, J=1.12 Hz, 1H), 3.85 (s, 1H), 2.54-2.49 (m, 2H), 0.91 (t, J=7.2 Hz, 3H). LCMS: (ES) C₁₂H1202 requires 188, found 189 [M+H]⁺.

Step 6: To a stirred solution of 1-(benzofuran-6-yl)butan-2-one (8-7) (6.0 g, 31.91 mmol, 1.0 eq.) in methanol (30 mL), methyl amine (79.78 mL, 2M in methanol, 159.57 mmol, 5.0 eq.) was added followed by the addition of catalytic amount of AcOH (1.0 mL). After stirring for 15 min, NaCNBH3 (56.03 g, 95.74 mmol, 3.0 eq.) was added to it. The resultant mixture was stirred at room temperature for 17 h. After completion [monitored by TLC, mobile Phase 10% MeOH-EtOAc], the excess solvent was evaporated under reduced pressure and basified by sodium carbonate solution (60 mL) then extracted with DCM (2×200 mL). Then dried over magnesium sulphate and concentrated under reduced pressure to obtained crude 1-(benzofuran-6-yl)-N-methylbutan-2-amine (6-MBPB) (5.0 g, 77%) which was forwarded to the next step without purification. ¹H NMR (400 MHz, DMSO-d₆): δ 7.90 (d, J=2.08 Hz, 1H), 7.54 (d, J=7.88 Hz, 1H), 7.40 (s, 1H), 7.09 (d, J=7.8 Hz, 1H), 6.89 (d, J=1.08 Hz, 1H), 2.77-2.72 (m, 1H), 2.67-2.62 (m, 1H), 2.58-2.53 (m, 1H), 2.26 (s, 3H), 1.35-1.23 (m, 2H), 0.84 (t, J=7.36 Hz, J=7.40 Hz, 3H). LCMS: (ES) C13H17NO requires 203, found 204.43 [M+H]⁺.

### Synthesis 16. Synthesis of Bk-5-MAPB HCl

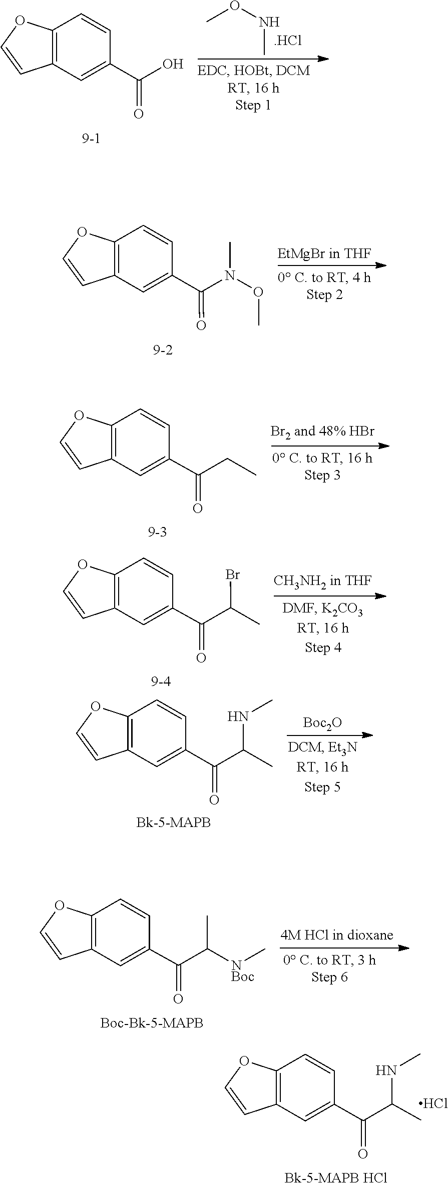

Step 1: Synthesis of N-Methoxy-N-methylbenzofuran-5-carboxamide (9-2): To a stirred solution of benzofuran-5-carboxylic acid (9-1) (10 g, 61.72 mmol, 1 eq.) in dry DCM (100 ml) was added DIPEA (32 ml, 185.18 mmol, 3 eq.) followed by EDC.HCl (13 g, 67.90 mmol, 1.1 eq.) and HOBT (12.5 g, 92.59 mmol, 1.5 eq.) under N2 atmosphere at room temperature and the resulting reaction mixture was allowed to stir at room temperature for 15 minutes. Then, N, O-dimethylhydroxylamine hydrochloride (6.62 g, 67.90 mmol, 1.1 eq.) was added to the resulting reaction mixture and was allowed to stir at room temperature for 16 hours. Completion of the reaction was monitored by TLC (20% EA in hexane). Upon completion, the reaction mixture was extracted with DCM twice (2×200 ml) and washed with water followed by brine solution. The combined organic layers were dried over anhydrous sodium sulphate, solvent was removed under vacuum and purified by silica gel column chromatography using ethyl acetate/hexane (20:80 v/v) as eluent to afford pure N-methoxy-N-methylbenzofuran-5-carboxamide (9-2) as yellow sticky gum (10.6 g, 83%). ¹H NMR (400 MHz, CDCl₃) δ 7.97 (s, 1H), 7.66 (m, 2H), 7.50 (d, J=8.56 Hz, 1H), 6.80 (d, J=1.08 Hz, 1H), 3.54 (s, 3H), 3.37 (s, 3H). LCMS: (ES) C11H11NO3 requires 205, found 206 [M+H]⁺.

Step 2: Synthesis of 1-(Benzofuran-5-yl) propan-1-one (9-3): To a stirred solution of N-methoxy-N-methylbenzofuran-5-carboxamide (9-2) (14 g, 68.22 mmol, 1 eq.) was added dry THE (250 ml) at 0° C. and was added 3 (M) solution of EtMgBr in diethyl ether (45 ml, 136.44 mmol, 2 eq.) to the reaction mixture and allowed to stir at room temperature for 4 hours. Upon completion of reaction (monitored by TLC, 20% EA in hexane) was quenched with saturated NH₄Cl solution and extracted with ethyl acetate, twice (2×100 ml), then washed with water followed by brine solution. The combined organic layers were dried over anhydrous sodium sulphate, solvent was evaporated under vacuum to afford crude compound 1-(benzofuran-5-yl) propan-1-one (9-3) as yellow solid (10 g, 84%). ¹H NMR (400 MHz, CDCl₃) δ 8.25 (d, J=1.48 Hz, 1H), 7.97 (dd, J=1.72 Hz, 8.72 Hz, 1H), 7.67 (d, J=6.68 Hz, 1H), 7.53 (d, J=8.72 Hz, 1H), 6.84 (d, J=1.56 Hz, 1H), 3.08 (q, 2H), 1.24 (t, J=7.24 Hz, 3H). LCMS: (ES) C₁₁H₁₀O₂ requires 174, found 175 [M+H]⁺.

Step 3: Synthesis of 1-(Benzofuran-5-yl)-2-bromopropan-1-one (9-4): To a stirred solution of 1-(benzofuran-5-yl)propan-1-one (9-3) (9 g, 51.66 mmol, 1 eq.) in dry THE (90 ml) was added hydrobromic acid 48% in water (133 ml, 1653.27 mmol, 32 eq.) and bromine (2.91 ml, 56.83 mmol, 1.1 eq.) dropwise at 0° C. and the reaction mixture was allowed to stir at room temperature for 16 hours. Upon completion, the reaction mixture (monitored by TLC, 10% EA in hexane) was quenched with saturated sodium carbonate solution, extracted with ethyl acetate (2×100 ml), and washed with water and brine solution. The combined organic layers were dried over anhydrous sodium sulphate, solvent was evaporated under vacuum and purified by silica gel column chromatography using ethyl acetate/hexane (10:90 v/v) as eluent to afford pure compound 1-(benzofuran-5-yl)-2-bromopropan-1-one (9-4) as yellow sticky gum (9 g, 68%). ¹H NMR (400 MHz, CDCl₃) δ 8.32 (d, J=1.52 Hz, 1H), 8.02 (dd, J=1.76 Hz, 8.72 Hz, 1H), 7.69 (d, J=2.2 Hz, 1H), 7.57 (d, J=8.72 Hz, 1H), 6.86 (d, J=1.96 Hz, 1H), 5.39 (q, 1H), 1.93 (t, J=6.6 Hz, 3H). LCMS: (ES) C₁₁H₁₉BrO₂ requires 253, found 254 [M+H]⁺.

Step 4: Synthesis of 1-(Benzofuran-5-yl)-2-(methylamino) propan-1-one (9-5): To a stirred solution of 1-(benzofuran-5-yl)-2-bromopropan-1-one (9-4) (9 g, 35.57 mmol, 1 eq.) in dry DMF (90 ml) was added potassium carbonate (7.36 g, 53.36 mmol, 1.5 eq.) and methyl amine 2(M) in THE (106.5 ml, 213.43 mmol, 6 eq.) in a sealed round bottom flask and the resulting reaction mixture was allowed to stir at room temperature for 16 hours. Upon completion of reaction (monitored by TLC, 10% EA in hexane) the crude was extracted with ethyl acetate (2×100 ml), and washed with water (2×100 ml) and brine solution. The combined organic solvent was dried over anhydrous sodium sulphate and solvent was evaporated under vacuum to afford crude 1-(benzofuran-5-yl)-2-(methylamino) propan-1-one (9-5) as yellow sticky gum (5.4 g, 74%). ¹H NMR (400 MHz, CDCl₃) δ 8.27 (s, 1H), 7.98 (dd, J=1.52 Hz, 8.68 Hz, 1H), 7.69 (d, J=2 Hz, 1H), 7.57 (d, J=8.56 Hz, 1H), 6.86 (s, 1H), 4.31 (q, 1H), 2.38 (s, 3H), 1.33 (d, J=7 Hz, 3H). LCMS: (ES) C₁₂H₁₃NO₂ requires 203, found 204 [M+H]⁺.

Step 5: Synthesis of tert-Butyl (1-(benzofuran-5-yl)-1-oxopropan-2-yl) (methyl) carbamate (Boc-Bk-5-MAPB): To a stirred solution of 1-(benzofuran-5-yl)-2-(methylamino) propan-1-one (9-5) (5.2 g, 25.61 mmol, 1 eq.) in dry DCM (50 ml) was added triethylamine (7.39 ml, 51.23 mmol, 2 eq.) and Boc anhydride (11.75 ml, 51.23 mmol, 2 eq.) and the resulting reaction mixture was allowed to stir at room temperature for 4 hours. Upon completion of reaction (monitored by TLC, 10% EA in hexane), the reaction mixture was extracted with DCM (2×100 ml) and washed with water followed by brine solution. Combined organic solvent was dried over anhydrous sodium sulphate and solvent was evaporated under vacuum and purified by silica gel column chromatography using ethyl acetate/hexane (10:90 v/v) as eluent to afford pure tert-butyl (1-(benzofuran-5-yl)-1-oxopropan-2-yl)(methyl)carbamate (Boc-Bk-5-MAPB) as yellow sticky gum (3.9 g, 50%). ¹H NMR (400 MHz, CDCl₃) δ 8.33 (s, 1H), 7.99 (d, J=8.52 Hz, 1H), 7.66 (bs, 1H), 7.52 (d, J=8.56 Hz, 1H), 6.81 (d, J=1.12 Hz, 1H), 5.80 (q, 1H), 2.59 (s, 3H), 1.43 (s, 9H), 1.37 (m, 3H). LCMS: (ES) C₁₇H₂₁NO₄ requires 303, found 304 [M+H]⁺.

Step 6: Synthesis of 1-(Benzofuran-5-yl)-2-(methylamino) propan-1-one hydrochloride (Bk-5-MAPB HCl): To a stirred solution of tert-butyl (1-(benzofuran-5-yl)-1-oxopropan-2-yl)(methyl) carbamate (Boc-Bk-5-MAPB) (1.8 g, 5.94 mmol, 1 eq.) in dry DCM (15 ml) was added 4(M) HCl in 1,4 dioxane (15 ml) at 0° C. and the resulting reaction mixture was allowed to stir at room temperature for 3 hours. Upon completion of reaction (monitored by TLC, 10% EA in hexane), the solvents were evaporated, the crude was washed twice with diethyl ether (2×50 ml) and pentane, and them dried under vacuum to afford 1-(benzofuran-5-yl)-2-(methylamino)propan-1-one hydrochloride (Bk-5-MAPB HCl) (1.3 g, 91%) as off white solid. ¹HNMR(400 MHz, CDCl₃) δ 10.52 (bs, 1H), 9.28 (bs, 1H), 8.26 (bs, 1H), 7.93 (d, J=8.32 Hz, 1H), 7.71 (d, J=1.72 Hz, 1H), 7.58 (bd, J=9.12 Hz, 1H), 6.86 (bs, 1H), 5.08 (bs, 1H), 2.87 (s, 3H), 1.82 (q, 3H). LCMS: (ES) C₁₂H₁₃NO₂ requires 203, found 204 [M+H]⁺. HPLC: Purity (λ 220 nm): 98.40%.

### Synthesis 17. Synthesis of Bk-6-MAPB HCl

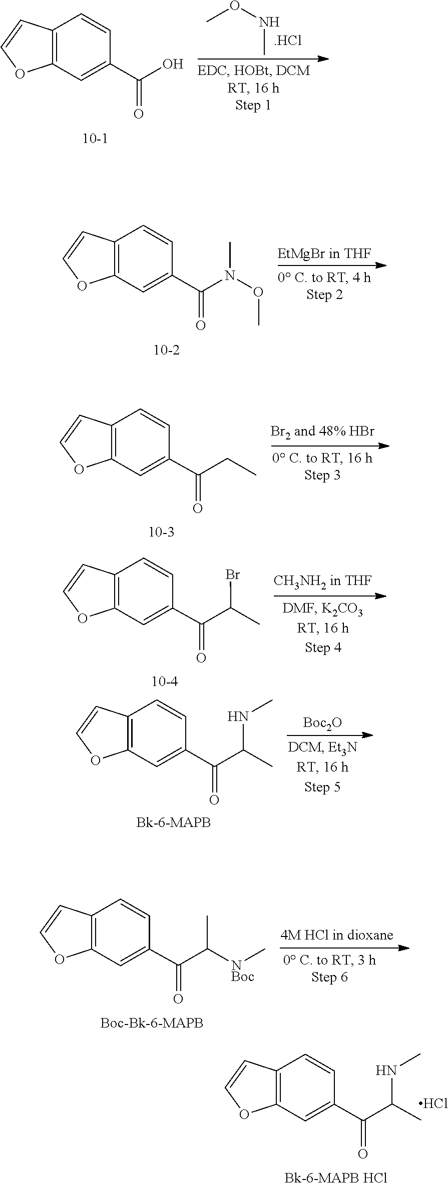

Step 1: Synthesis of N-methoxy-N-methylbenzofuran-6-carboxamide (10-2): To a stirred solution of benzofuran-6-carboxylic acid (10-1) (10 g, 61.72 mmol, 1 eq.) in dry DCM (100 ml) was added DIPEA (32 ml, 185.18 mmol, 3 eq.) followed by EDC.HCl (13 g, 67.90 mmol, 1.1 eq.) and HOBT (12.5 g, 92.59 mmol, 1.5 eq.) under N2 atmosphere at room temperature and the resulting reaction mixture was allowed to stir at room temperature for 15 minutes. Then N, O-dimethylhydroxylamine hydrochloride (6.62 g, 67.90 mmol, 1.1 eq.) was added to the resulting reaction mixture and was allowed to stir at room temperature for 16 hours. Completion of the reaction was monitored by TLC (20% EA in hexane). Upon completion, the reaction mixture was extracted with DCM twice (2×200 ml) and washed with water followed by brine solution. The combined organic layers were dried over anhydrous sodium sulphate, solvent was removed under vacuum and purified by silica gel column chromatography using ethyl acetate/hexane (20:80 v/v) as eluent to afford pure N-methoxy-N-methylbenzofuran-6-carboxamide (10-2) as yellow sticky gum (11.4 g, 90%). ¹H NMR (400 MHz, CDCl₃) δ 7.88 (bs, 1H), 7.70 (d, J=2.08 Hz, 1H), 7.60 (s, 2H), 6.79 (d, J=1.16 Hz, 1H), 3.55 (s, 3H), 3.38 (s, 3H). LCMS: (ES) C₁₁H₁₁NO₃ requires 205, found 206 [M+H]⁺.

Step 2: Synthesis of 1-(benzofuran-6-yl) propan-1-one (10-3): To a stirred solution of N-methoxy-N-methylbenzofuran-6-carboxamide (10-2) (10 g, 48.73 mmol, 1 eq.) was added dry THF (150 ml) at 0° C. and followed by 3(M) solution of EtMgBr in diethyl ether (32.4 ml, 97.46 mmol, 2 eq.) to the reaction mixture and allowed to stir at room temperature for 4 hours. Upon completion, the reaction (monitored by TLC, 20% EA in hexane) was quenched with saturated NH₄Cl solution and extracted with ethyl acetate twice (2×100 ml), and washed with water and brine solution. The combined organic layers were dried over anhydrous sodium sulphate and solvent was evaporated under vacuum to afford crude compound 1-(benzofuran-6-yl) propan-1-one (10-3) as yellow solid (7 g, 82%). ¹H NMR (400 MHz, CDCl₃) δ 8.12 (s, 1H), 7.90 (d, J=8.24 Hz, 1H), 7.76 (d, J=1.96 Hz, 1H), 7.65 (d, J=8.24 Hz, 1H), 6.81 (t, J=0.76 Hz & 0.92 Hz, 1H), 3.08 (q, 2H), 1.25 (t, J=7.28 Hz & 7.24 Hz, 3H). LCMS: (ES) C₁₁H₁₀O₂ requires 174, found 175 [M+H]⁺.

Step 3: Synthesis of 1-(benzofuran-6-yl)-2-bromopropan-1-one (10-4): To a stirred solution of 1-(benzofuran-6-yl)propan-1-one (10-3) (3 g, 17.22 mmol, 1 eq.) in dry THE (30 ml) was added hydrobromic acid 48% in water (30 ml, 551 mmol, 32 eq.) and bromine (0.97 ml, 18.94 mmol, 1.1 eq.) dropwise at 0° C. and the reaction mixture was allowed to stir at room temperature for 16 hours. Upon completion of the reaction (monitored by TLC, 10% EA in hexane), the reaction mixture was quenched with saturated sodium carbonate solution, extracted with ethyl acetate (2×100 ml), and washed with water and brine solution. The combined organic layers were dried over anhydrous sodium sulphate and solvent was evaporated under vacuum and purified by silica gel column chromatography using ethyl acetate/hexane (10:90 v/v) as eluent to afford pure compound 1-(benzofuran-6-yl)-2-bromopropan-1-one (10-4) as a yellow sticky gum (1.9 g, 43.6%). ¹H NMR (400 MHz, CDCl₃) δ 8.20 (bs, 1H), 7.94 (bd, J=8.16 Hz, 1H), 7.80 (d, J=2 Hz, 1H), 7.68 (bd, J=8.2 Hz, 1H), 6.83 (bs, 1H), 5.37 (q, 1H), 1.93 (d, J=6.68 Hz, 3H). LCMS: (ES) C₁₁H₁₉BrO₂ requires 252, found 253 [M+H]⁺.

Step 4: Synthesis of 1-(benzofuran-6-yl)-2-(methylamino) propan-1-one (Bk-6-MAPB): To a stirred solution of 1-(benzofuran-6-yl)-2-bromopropan-1-one (16-4) (3.8 g, 15 mmol, 1 eq.) in dry DMF (30 ml) was added potassium carbonate (3.1 g, 22.53 mmol, 1.5 eq.) and methyl amine 2(M) in THE (45 ml, 90.11 mmol, 6 eq.) in a sealed round bottom flask and the resulting reaction mixture was allowed to stir at room temperature for 16 hours. Upon completion of the reaction (monitored by TLC, 10% EA in hexane), the crude was extracted with ethyl acetate (2×50 ml) and washed with water (2×50 ml) and brine solution. The combined organic solvent was dried over anhydrous sodium sulphate and solvent was evaporated under vacuum to afford crude 1-(benzofuran-6-yl)-2-(methylamino) propan-1-one (Bk-6-MAPB) as a yellow sticky gum (3 g, 98%). ¹H NMR (400 MHz, CDCl₃) δ 8.14 (s, 1H), 7.90 (d, J=8.2 Hz, 1H), 7.78 (d, J=1.96 Hz, 1H), 7.68 (d, J=8.2 Hz, 1H), 6.83 (s, 1H), 4.29 (q, 1H), 2.38 (s, 3H), 1.34 (d, J=6.96 Hz, 3H). LCMS: (ES) C₁₂H₁₃NO₂ requires 203, found 204 [M+H]⁺.

Step 5: Synthesis of tert-butyl (1-(benzofuran-6-yl)-1-oxopropan-2-yl) (methyl) carbamate (Boc-Bk-6-MAPB): To a stirred solution of 1-(benzofuran-6-yl)-2-(methylamino) propan-1-one (Bk-6-MAPB) (3 g, 14.77 mmol, 1 eq.) in dry DCM (30 ml) was added triethylamine (4.26 ml, 29.55 mmol, 2 eq.) and Boc anhydride (6.78 ml, 29.55 mmol, 2 eq.) and the resulting reaction mixture was allowed to stir at room temperature for 4 hours. Upon completion of the reaction (monitored by TLC, 10% EA in hexane), the reaction mixture was extracted with DCM (2×50 ml) and washed with water followed by brine solution. The combined organic solvent was dried over anhydrous sodium sulphate and the solvent was evaporated under vacuum and purified by silica gel column chromatography using ethyl acetate/hexane (10:90 v/v) as eluent to afford tert-butyl (1-(benzofuran-6-yl)-1-oxopropan-2-yl)(methyl) carbamate (Boc-Bk-6-MAPB) as a yellow sticky gum (2.5 g, 55%). 1H NMR (400 MHz, CDCl₃) δ 8.20-8.11 (bs, 1H), 7.93-7.85 (bd, 1H), 7.76 (s, 1H), 7.63 (bs, 1H), 6.80 (s, 1H), 5.77-5.31 (m, 1H), 2.76-2.58 (s, 3H), 1.45 (s, 9H), 1.38 (m, 3H). Rotamers observed. LCMS: (ES) C₁₇H₂₁NO₄ requires 303, found 304 [M+H]⁺.

Step 6: Synthesis of 1-(benzofuran-6-yl)-2-(methylamino) propan-1-one hydrochloride (Bk-6-MAPB HCl): To a stirred solution of tert-butyl (1-(benzofuran-6-yl)-1-oxopropan-2-yl)(methyl) carbamate (Boc-Bk-6-MAPB) (1.5 g, 4.95 mmol, 1 eq.) in dry DCM (15 ml) was added 4(M) HCl in 1,4 dioxane (15 ml) at 0° C. and the resulting reaction mixture was allowed to stir at room temperature for 3 hours. Upon completion of reaction (monitored by TLC, 10% EA in hexane), the solvent was evaporated, and the crude was washed twice with diethyl ether (2×50 ml) and pentane and dried under vacuum to afford 1-(benzofuran-6-yl)-2-(methylamino)propan-1-one hydrochloride (HCl Bk-6-MAPB) (1.1 g, 92%) as off white solid. ¹HNMR (400 MHz, CDCl₃) δ 10.90 (s, 1H), 8.92 (s, 1H), 8.13 (s, 1H), 7.84 (bd, J=6.88 Hz, 1H), 7.72 (bd, J=8.16 Hz, 1H), 6.86 (s, 1H), 4.96 (bs, 1H), 2.86 (s, 3H), 1.85 (d, J=7.08 Hz, 3H). LCMS: (ES) C₁₂H₁₃NO₂ requires 203, found 204 [M+H]⁺. HPLC: Purity (λ 220 nm): 99.85%.

### Synthesis 18. Synthesis of Bk-5-MBPB HCl

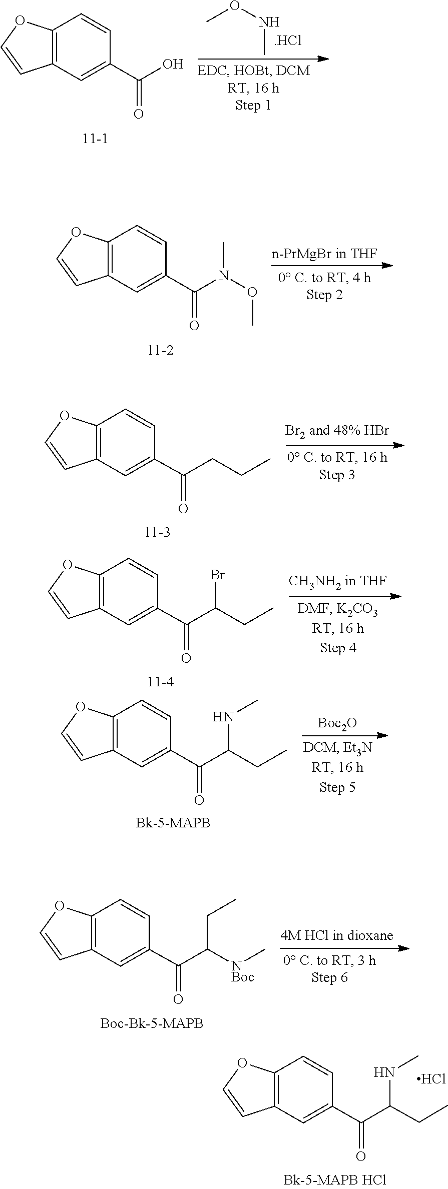

Step 1: Synthesis of N-methoxy-N-methylbenzofuran-5-carboxamide (11-2): To a stirred solution of benzofuran-5-carboxylic acid (11-1) (10 g, 61.72 mmol, 1 eq.) in dry DCM (100 ml) was added DIPEA (32 ml, 185.18 mmol, 3 eq.) followed by EDC.HCl (13 g, 67.90 mmol, 1.1 eq.) and HOBT (12.5 g, 92.59 mmol, 1.5 eq.) under N2 atmosphere at room temperature and the resulting reaction mixture was allowed to stir at room temperature for 15 minutes. Then N, O-dimethylhydroxylamine hydrochloride (6.62 g, 67.90 mmol, 1.1 eq.) was added to the resulting reaction mixture and was allowed to stir at room temperature for 16 hours. Upon completion, monitored by TLC (20% EA in hexane), the reaction mixture was extracted with DCM twice (2×200 ml) and washed with water followed by brine solution. The combined organic layers were dried over anhydrous sodium sulphate and solvent was removed under vacuum and purified by silica gel column chromatography using ethyl acetate/hexane (20:80 v/v) as eluent to afford pure N-methoxy-N-methylbenzofuran-5-carboxamide (11-2) as yellow sticky gum (10.6 g, 83%). ¹H NMR (400 MHz, CDCl₃) δ 7.97 (s, 1H), 7.66 (m, 2H), 7.50 (d, J=8.56 Hz, 1H), 6.80 (d, J=1.08 Hz, 1H), 3.54 (s, 3H), 3.37 (s, 3H). LCMS: (ES) C₁₁H₁₁NO₃ requires 205, found 206 [M+H]⁺.

Step 2: Synthesis of 1-(benzofuran-5-yl) butan-1-one (11-3): To a stirred solution of N-methoxy-N-methylbenzofuran-5-carboxamide (11-2) (5 g, 24.37 mmol, 1 eq.) was added in dry THF (50 ml) at 0° C. and was added 2 (M) solution of n-propylMgBr in THE (24.4 ml, 48.73 mmol, 2 eq.) to the reaction mixture and allowed to stir at room temperature for 4 hours. Upon completion, (monitored by TLC, 20% EA in hexane) the reaction was quenched with saturated NH₄Cl solution and extracted with ethyl acetate twice (2×75 ml) and then washed with water followed by brine solution. The combined organic layers were dried over anhydrous sodium sulphate, solvent was evaporated under vacuum to afford crude compound 1-(benzofuran-5-yl) butan-1-one (11-3) as yellow solid (4.5 g, 98%).¹H NMR (400 MHz, CDCl₃) δ 8.25 (d, J=1.56 Hz, 1H), 7.97 (dd, J=1.72 Hz, 8.72 Hz, 1H), 7.67 (d, J=2.2 Hz, 1H), 7.53 (d, J=8.72 Hz, 1H), 6.84 (d, J=1.88 Hz, 1H), 2.99 (t, J=7.28 Hz, 7.36 Hz, 2H), 1.83 (q, 2H), 1.01 (t, J=7.4 Hz, 3H). LCMS: (ES) C₁₂H₁₂O₂ requires 188, found 189 [M+H]⁺.

Step 3: Synthesis of 1-(benzofuran-5-yl)-2-bromobutan-1-one (11-4): To a stirred solution of 1-(benzofuran-5-yl)butan-1-one (11-3) (3 g, 15.95 mmol, 1 eq.) in dry THE (30 ml) was added hydrobromic acid 48% in water (41.3 ml, 510.63 mmol, 32 eq.) and bromine (0.89 ml, 17.55 mmol, 1.1 eq.) dropwise at 0° C. and the reaction mixture was allowed to stir at room temperature for 16 hours. Upon completion, (monitored by TLC, 10% EA in hexane), the reaction mixture was quenched with saturated sodium carbonate solution, extracted with ethyl acetate (2×50 ml), and washed with water and brine solution. The combined organic layers were dried over anhydrous sodium sulphate and solvent was evaporated under vacuum and purified by silica gel column chromatography using ethyl acetate/hexane (10:90 v/v) as eluent to afford pure compound 1-(benzofuran-5-yl)-2-bromobutan-1-one (11-4) as yellow sticky gum (3.2 g, 75%). ¹H NMR (400 MHz, CDCl₃) δ 8.32 (d, J=1.32 Hz, 1H), 8.02 (dd, J=1.52 Hz, 8.72 Hz, 1H), 7.70 (d, J=2.08 Hz, 1H), 7.57 (d, J=8.72 Hz, 1H), 6.87 (d, J=1.8 Hz, 1H), 5.14 (t, J=7.04 Hz, 7.08 Hz, 1H), 2.30 (m, 2H), 1.09 (t, J=7.64 Hz, 7.28 Hz, 3H). LCMS: (ES) C₁₂H₁₁BrO₂ requires 267, found 268 [M+H]⁺

Step 4: Synthesis of 1-(benzofuran-5-yl)-2-(methylamino) butan-1-one (11-5): To a stirred solution of 1-(benzofuran-5-yl)-2-bromobutan-1-one (11-4) (3.2 g, 11.98 mmol, 1 eq.) in dry DMF (30 ml) was added potassium carbonate (2.48 g, 17.97 mmol, 1.5 eq.) and methyl amine 2(M) in THE (36 ml, 71.91 mmol, 6 eq.) in a sealed round bottom flask and the resulting reaction mixture was allowed to stir at room temperature for 16 hours. Upon completion of the reaction (monitored by TLC, 10% EA in hexane), volatiles were evaporated, and the crude was extracted with ethyl acetate (2×50 ml) and washed with water (2×50 ml) and brine solution. The combined organic solvent was dried over anhydrous sodium sulphate and solvent was evaporated under vacuum to afford crude 1-(benzofuran-5-yl)-2-(methylamino) butan-1-one (Bk-5-MBPB) as yellow sticky gum (2.3 g, 88%). ¹H NMR (400 MHz, CDCl₃) δ 8.26 (d, J=1.12 Hz, 1H), 7.98 (dd, J=1.40 Hz, 8.64 Hz, 1H), 7.69 (d, J=1.96 Hz, 1H), 7.57 (d, J=8.6 Hz, 1H), 6.86 (d, J=1.16 Hz, 1H), 4.15 (t, J=5.76 Hz, 5.80 Hz, 1H), 2.37 (s, 3H), 1.86 (m, 1H), 1.63 (m, 1H), 0.92 (t, J=7.44 Hz, 3H). LCMS: (ES) C₁₃H₁₅NO₂ requires 217, found 218 [M+H]⁺.

Step 5: Synthesis of tert-butyl (1-(benzofuran-5-yl)-1-oxobutan-2-yl) (methyl) carbamate (Boc-Bk-5-MBPB): To a stirred solution of 1-(benzofuran-5-yl)-2-(methylamino) butan-1-one (Bk-5-MBPB) (2.3 g, 10.59 mmol, 1 eq.) in dry DCM (30 ml) was added triethylamine (3.05 ml, 21.19 mmol, 2 eq.) and Boc anhydride (4.86 ml, 21.19 mmol, 2 eq.) and the resulting reaction mixture was allowed to stir at room temperature for 4 hours. Upon completion, (monitored by TLC, 10% EA in hexane), the reaction mixture was extracted with DCM (2×50 ml) and washed with water followed by brine solution. Combined organic solvent was dried over anhydrous sodium sulphate, solvent was evaporated under vacuum and purified by silica gel column chromatography using ethyl acetate/hexane (10:90 v/v) as eluent to afford pure tert-butyl (1-(benzofuran-5-yl)-1-oxobutan-2-yl)(methyl)carbamate (Boc-Bk-5-MBPB) as a yellow sticky gum (1.7 g, 50%).¹H NMR (400 MHz, CDCl₃) δ 8.38 (s, 1H), 8.03 (dd, J=8.76 Hz, 1H), 7.68 (m, 1H), 7.52 (d, J=4.8 Hz, 1H), 6.82 (s, 1H), 5.62 (m, 1H), 2.67 (s, 3H), 1.97 (m, 1H), 1.78 (m, 1H), 1.52 (s, 9H), 0.96 (m, 3H). Rotamer observed. LCMS: (ES) C₁₈H₂₃NO₄ requires 317, found 318 [M+H]⁺.

Step 6: Synthesis of 1-(benzofuran-5-yl)-2-(methylamino)butan-1-one hydrochloride (Bk-5-MBPB HCl): To a stirred solution of tert-butyl (1-(benzofuran-5-yl)-1-oxobutan-2-yl)(methyl) carbamate (Boc-Bk-5-MBPB) (1.5 g, 4.73 mmol, 1 eq.) in dry DCM (15 ml) was added 4(M) HCl in 1,4 dioxane (15 ml) at 0° C. and the resulting reaction mixture was allowed to stir at room temperature for 3 hours. Upon completion of reaction (monitored by TLC, 10% EA in hexane), the solvent was evaporated, and the crude was washed twice with diethyl ether (2×30 ml) and pentane and dried under vacuum to afford 1-(benzofuran-5-yl)-2-(methylamino)butan-1-one hydrochloride (HCl Bk-5-MBPB) (1.15 g, 95%) as off white solid. ¹H NMR (400 MHz, CDCl₃) δ 10.51 (s, 1H), 9.10 (s, 1H), 8.31 (s, 1H), 7.97 (d, J=8.32 Hz, 1H), 7.72 (s, 1H), 7.60 (d, J=8.32 Hz, 1H), 6.88 (s, 1H), 5.12 (s, 1H), 2.86 (s, 3H), 2.41 (bs, 1H), 2.22 (bs, 1H), 1.87 (s, 2H), 1.03 (t, J=6.28 Hz, 6.48 Hz, 3H). LCMS: (ES) C₁₃H₁₅NO₂ requires 217, found 218 [M+H]⁺. HPLC: Purity (λ 220 nm): 96.94%.

### Synthesis 19. Synthesis of Bk-6-MBPB HCl

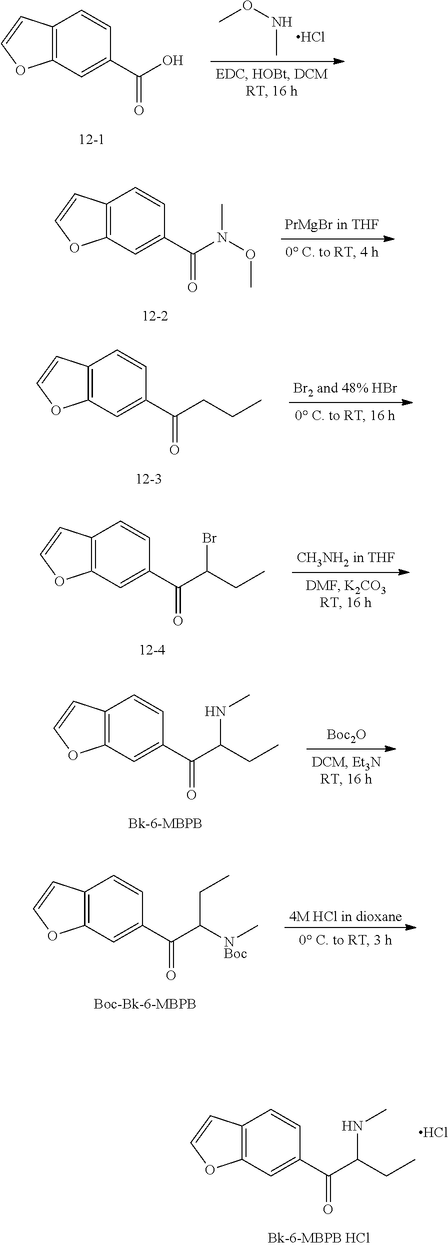

Step 1: Synthesis of N-methoxy-N-methylbenzofuran-6-carboxamide (12-2): To a stirred solution of benzofuran-6-carboxylic acid (12-1) (10 g, 61.72 mmol, 1 eq.) in dry DCM (100 mL) was added DIPEA (32 ml, 185.18 mmol, 3 eq.) followed by EDC.HCl (13 g, 67.90 mmol, 1.1 eq.) and HOBT (12.5 g, 92.59 mmol, 1.5 eq.) under N2 atmosphere at room temperature and the resulting reaction mixture was allowed to stir at room temperature for 15 minutes. Then N, O-dimethylhydroxylamine hydrochloride (6.62 g, 67.90 mmol, 1.1 eq.) was added to the resulting reaction mixture and was allowed to stir at room temperature for 16 hours. Upon completion (monitored by TLC 20% EA in hexane), the reaction mixture was extracted with DCM twice (2×200 ml) and washed with water followed by brine solution. The combined organic layers were dried over anhydrous sodium sulphate and solvent was removed under vacuum and purified by silica gel column chromatography using ethyl acetate/hexane (20:80 v/v) as eluent to afford pure N-methoxy-N-methylbenzofuran-6-carboxamide (12-2) as yellow sticky gum (10.6 g, 83%). ¹H NMR (400 MHz, CDCl₃) δ 7.97 (bs, 1H), 7.66 (m, 2H), 7.50 (d, J=8.56 Hz, 1H), 6.80 (s, 1H), 3.54 (s, 3H), 3.37 (s, 3H). LCMS: (ES) C₁₁H₁₁NO₃ requires 205, found 206 [M+H]⁺.

Step 2: Synthesis of 1-(benzofuran-6-yl)butan-1-one (12-3): To a stirred solution of N-methoxy-N-methylbenzofuran-6-carboxamide (12-2) (10 g, 48.73 mmol, 1 eq.) was added dry THF (100 mL) at 0° C. and 2 (M) solution of n-propylmagnesium bromide in THE (48.73 mL, 97.46 mmol, 2 eq.). The reaction mixture and allowed to stir at room temperature for 4 hours. Upon completion of reaction (monitored by TLC, 20% EA in hexane) was quenched with saturated NH₄Cl solution and extracted with ethyl acetate twice (2×200 ml), and then washed with water followed by brine solution. The combined organic layers were dried over anhydrous sodium sulphate and solvent was evaporated under vacuum to afford crude 1-(benzofuran-6-yl)butan-1-one (12-3) as yellow solid (9 g, 98%).¹H NMR (400 MHz, CDCl₃) δ 8.11 (s, 1H), 7.89 (d, J=8.2 Hz, 1H), 7.76 (d, J=2.04 Hz, 1H), 7.64 (d, J=8.16 Hz, 1H), 6.81 (d, J=1.3 Hz, 1H), 3.04 (m, 2H), 1.84 (m, 2H), 1.03 (t, J=7.4 Hz, 3H). LCMS: (ES) C₁₂H₁₂O₂ requires 188, found 189 [M+H]⁺.

Step 3: Synthesis of 1-(benzofuran-6-yl)-2-bromobutan-1-one (12-4): To a stirred solution of 1-(benzofuran-6-yl)butan-1-one (12-3) (4.6 g, 24.46 mmol, 1 eq.) in dry THE (50 mL) was added hydrobromic acid 48% in water (42.51 ml, 782.97 mmol, 32 eq.) and bromine (1.37 mL, 26.91 mmol, 1.1 eq.) dropwise at 0° C. and the reaction mixture was allowed to stir at room temperature for 16 hours. Upon completion, the reaction mixture (monitored by TLC, 10% EA in hexane) was quenched with saturated sodium carbonate solution, extracted with ethyl acetate (2×100 ml), and washed with water and brine solution. The combined organic layers were dried over anhydrous sodium sulphate, solvent was evaporated under vacuum and purified by silica gel column chromatography using ethyl acetate/hexane (10:90 v/v) as eluent to afford pure 1-(benzofuran-6-yl)-2-bromobutan-1-one (12-4) as yellow sticky gum (3.8 g, 58%). ¹H NMR (400 MHz, CDCl₃) δ 8.18 (s, 1H), 7.93 (d, J=7.16 Hz, 1H), 7.80 (d, J=2.08 Hz, 1H), 7.68 (d, J=8.04 Hz, 1H), 6.83 (s, 1H), 5.12 (t, J=7.12 Hz, 6.72 Hz, 1H), 2.28 (m, 2H), 1.09 (t, J=7.28 Hz, 7.32 Hz, 3H). LCMS: (ES) C₁₂H₁₁BrO₂ requires 267, found 268 [M+H]⁺.

Step 4: Synthesis of 1-(benzofuran-6-yl)-2-(methylamino)butan-1-one (Bk-6-MBPB): To a stirred solution of 1-(benzofuran-6-yl)-2-bromobutan-1-one (12-4) (3.8 g, 14.22 mmol, 1 eq.) in dry DMF (40 mL) was added potassium carbonate (2.94 g, 21.33 mmol, 1.5 eq.) and methyl amine 2(M) in THE (42.5 mL, 85.37 mmol, 6 eq.) in a sealed round bottom flask and the resulting reaction mixture was allowed to stir at room temperature for 16 h. Upon completion of reaction (monitored by TLC, 10% EA in Hexane), volatiles were evaporated, and the crude was extracted with ethyl acetate (2×100 ml), washed with water (2×50 ml) and brine solution. Combined organic solvent was dried over anhydrous sodium sulphate, solvent was evaporated under vacuum to afford crude 1-(benzofuran-6-yl)-2-(methylamino)butan-1-one (Bk-6-MBPB) as yellow sticky gum (2.75 g, 89%). Crude ¹H NMR (400 MHz, CDCl₃) δ 8.14 (s, 1H), 7.90 (d, J=0.96 Hz, 8.0 Hz, 1H), 7.79 (d, J=2.04 Hz, 1H), 7.68 (d, J=8.2 Hz, 1H), 6.83 (d, J=1 Hz, 1H), 4.14 (t, J=6.36 Hz, 5.48 Hz, 1H), 2.37 (s, 3H), 1.86 (m, 1H), 1.60 (m, 1H), 0.92 (t, J=7.44 Hz, 3H). LCMS: (ES) C₁₃H₁₅NO₂ requires 217, found 218 [M+H]⁺.

Step 5: Synthesis of tert-butyl (1-(benzofuran-6-yl)-1-oxobutan-2-yl)(methyl)carbamate (Boc-Bk-6-MBPB): To a stirred solution of 1-(benzofuran-6-yl)-2-(methylamino)butan-1-one (Bk-6-MBPB) (2.75 g, 12.65 mmol, 1 eq.) in dry DCM (30 mL) was added triethylamine (3.65 mL, 25.31 mmol, 2 eq.) and Boc anhydride (5.8 mL, 25.31 mmol, 2 eq.) and the resulting reaction mixture was allowed to stir at room temperature for 4 hours. Upon completion of the reaction (monitored by TLC, 10% EA in hexane), the reaction mixture was extracted with DCM (2×50 ml) and washed with water followed by brine solution. The combined organic layers were dried over anhydrous sodium sulphate, solvent was evaporated under vacuum, and the crude material purified by silica gel column chromatography using ethyl acetate/hexane (10:90 v/v) as eluent to afford pure tert-butyl (1-(benzofuran-6-yl)-1-oxobutan-2-yl)(methyl)carbamate (Boc-Bk-6-MBPB) as yellow sticky gum (3.4 g, 84%).¹H NMR (400 MHz, CDCl₃) δ 8.24 (s, 1H), 7.97 (dd, J=8.2 Hz, 1H), 7.76 (bs, 1H), 7.63 (bm, 1H), 6.80 (bs, 1H), 5.61 (t, J=5.64 Hz, 8.88 Hz, 1H), 2.66 (s, 3H), 1.99 (q, 2H), 1.55 (s, 9H), 0.98 (m, 3H). Rotamer observed. LCMS: (ES) C₁₈H₂₃NO₄ requires 317, found 318 [M+H]⁺.

Step 6: Synthesis of 1-(benzofuran-6-yl)-2-(methylamino)butan-1-one hydrochloride Bk-6-MBPB HCl): To a stirred solution of tert-butyl (1-(benzofuran-6-yl)-1-oxobutan-2-yl)(methyl)carbamate (Boc-Bk-6-MBPB) (1.5 g, 4.73 mmol, 1 eq.) in dry DCM (15 mL) was added 4(M) HCl in 1,4 dioxane (15 mL) at 0° C. and the resulting reaction mixture was allowed to stir at room temperature for 3 hours. Upon completion of the reaction (monitored by TLC, 10% EA in hexane), the solvent were evaporated and the crude was washed twice with diethyl ether (2×50 ml) and pentane and dried under vacuum to afford 1-(benzofuran-6-yl)-2-(methylamino)butan-1-one hydrochloride (Bk-6-MBPB HCl) (1 g, 83%) as a white solid. ¹H NMR (400 MHz, CDCl₃) δ 10.78 (s, 1H), 8.95 (s, 1H), 8.15 (s, 1H), 7.87 (m, 2H), 7.72 (d, J=8.08 Hz, 1H), 6.86 (d, J=1.88 Hz, 1H), 4.99 (bs, 1H), 2.86 (bs, 3H), 2.48 (m, 1H), 2.71 (m, 1H), 1.05 (m, 3H). LCMS: (ES) C₁₃H₁₅NO₂ requires 217, found 218 [M+H]⁺. HPLC: Purity (k 300 nm): 99.68%.

### Synthesis 20. Synthesis of (R)-1-(benzofuran-5-yl)-N-methylpropan-2-amine (R-5-MAPB)

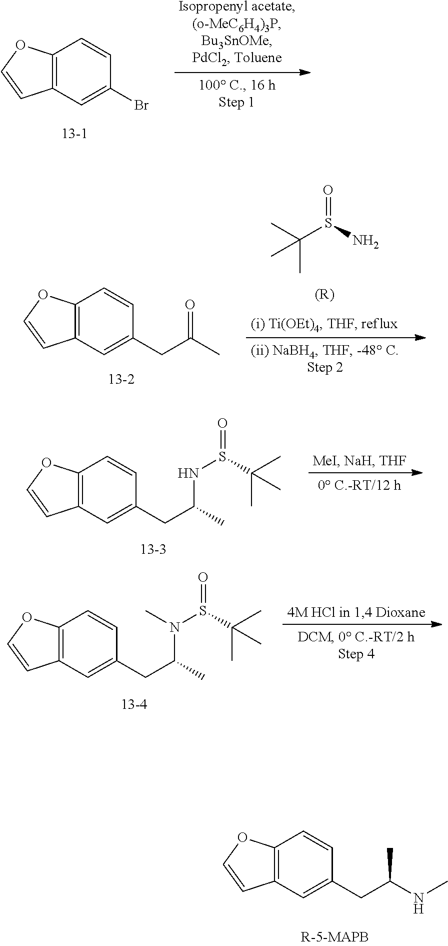

Step 1: To a stirred solution of 5-bromobenzofuran (13-1) (20 g, 101.52 mmol, 1 eq.) in dry Toluene (400 ml) was added tri(o-tolyl)phosphine (1.84 g, 6.091 mmol, 0.06 eq.), tributyl tin methoxide (48.89 mL, 152.28 mmol, 1.5 eq.) and Isopropenyl acetate (16.99 mL, 156.34 mmol, 1.54 eq.) and the resulting reaction mixture was degassed under nitrogen for 15 minutes. Then palladium (II) chloride (1.26 g, 7.10 mmol, 0.07 eq.) was added to the reaction mixture and the resulting reaction mixture was heated to 100° C. for 16 hrs. Upon completion, monitored by TLC (10% EA in Hexane), the reaction mixture was filtered through celite bed, extracted with ethyl acetate (2×400 ml), washed with water, followed by saturated potassium fluoride solution, and brine solution. Combined organic layer was dried over anhydrous sodium sulphate, solvent was removed under vacuum and purified by silica gel column chromatography using ethyl acetate/hexane (10:90 v/v) as eluent to afford 1-(benzofuran-5-yl)propan-2-one (13-2) as light yellow gum (17 g, 96%). ¹H NMR (400 MHz, DMSO-d₆) δ 7.96 (d, J=2.08 Hz, 1H), 7.53 (d, J=8.48 Hz, 1H), 7.46 (s, 1H), 7.13 (dd, J=1.52 Hz, 8.44 Hz, 1H), 6.92 (d, J=0.76 Hz, 1H), 3.83 (s, 2H), 2.12 (s, 3H). LCMS: (ES) C₁₁H₁₀O₂ requires 174, found 175 [M+H]⁺.

Step 2: To a stirred solution of 1-(benzofuran-5-yl)propan-2-one (13-2) (9 g, 51.66 mmol, 1 eq.) in dry THE (150 ml) was added Ti(OEt)₄ (37.91 ml, 180.82 mmol, 3.5 eq.) and (R)-2-methylpropane-2-sulfinamide (6.26 g, 51.66 mmol, 1 eq.) (dissolved in 30 ml dry THF) and the resulting reaction mixture was allowed to stir at 70° C. for 12 hrs. Upon completion, monitored by TLC (50% EA in Hexane), the reaction mixture was cooled to 0° C., gradually to −48° C. and NaBH₄ (7.81 g, 206.65 mmol, 4 eq.) (dissolved in 30 ml dry THF) was added into the reaction mixture at −48° C. and the resulting reaction mixture was allowed to stir at −48° C. for 3 hrs. Upon completion, monitored by TLC (50% EA in Hexane), the reaction mixture was taken to room temperature and was quenched with Methanol and Sat NaCl solution (until white precipitate observed). The reaction mixture was then filtered through celite bed, washed with methanol (2×150 ml) and ethyl acetate (2×150 ml), evaporated under vacuum to remove the volatiles. Then the reaction mixture was extracted with ethyl acetate, washed with water, followed by brine solution. Combined organic layer was dried over anhydrous sodium sulphate, solvent was removed under vacuum to afford crude (R)-N-((R)-1-(benzofuran-5-yl)propan-2-yl)-2-methylpropane-2-sulfinamide (13-3) as yellow sticky gum (14 g, 96%). ¹H NMR (400 MHz, DMSO-d₆) δ 7.94 (s, 1H), 7.48 (m, 2H), 7.15 (d, J=8.32 Hz, 1H), 6.89 (d, J=7.76 Hz, 1H), 4.97 (d, J=6.04 Hz, 1H), 3.48 (m, 1H), 3.07 (m, 1H), 2.76 (m, 1H), 1.09 (s, 12H), 1.08 (m, 3H) LCMS: (ES) C₁₅H₂₁NO₂S requires 279, found 280 [M+H]⁺.

Step 3: To a stirred solution of (R)-N-((R)-1-(benzofuran-5-yl)propan-2-yl)-2-methylpropane-2-sulfinamide (13-3) (15 g, 53.57 mmol, 1 eq.) in dry THE (100 mL) (In a sealed tube) was added NaH (60%) (4.28 g, 107.14 mmol, 2 eq.) at 0° C. and the resulting reaction mixture was allowed to stir at 0° C. for 30 min. Then Iodomethane (6.7 ml, 107.14 mmol, 2 eq.) was added at 0° C. and the resulting reaction mixture was allowed to stir at room temperature for 12 h. Upon completion, monitored by TLC (50% EA in Hexane), the reaction mixture was quenched with ice water, extracted with ethyl acetate (2×250 ml), washed with saturated ammonium chloride solution, followed by brine solution. Combined organic layer was dried over anhydrous sodium sulphate, solvent was removed under vacuum and purified by silica gel column chromatography using ethyl acetate/hexane (50:50 v/v) as eluent to afford (R)-N-((R)-1-(benzofuran-5-yl)propan-2-yl)-N,2-dimethylpropane-2-sulfinamide (13-4) as light yellow gum (8 g, 50.9%). ¹H NMR (400 MHz, DMSO-d₆) δ 7.93 (s, 1H), 7.49 (m, 2H), 7.14 (d, J=7.4, 1H), 6.89 (s, 1H), 3.54 (m, 1H), 2.92 (m, 1H), 2.81 (m, 1H), 2.49 (s, 3H), 1.09 (d, J=6.64 Hz, 3H), 1.02 (s, 9H). LCMS: (ES) C₁₆H₂₃NO₂S requires 293, found 294 [M+H]⁺.

Step 4: To a stirred solution of (R)-N-((R)-1-(benzofuran-5-yl)propan-2-yl)-N,2-dimethylpropane-2-sulfinamide (13-4) (10.5 g, 37.58 mmol, 1 eq.) in dry DCM (50 ml) was added 4M HCl in 1,4 dioxane (100 mL) at 0° C. and then the resulting reaction mixture was allowed to stir at room temperature for 2 h. Upon completion of reaction (monitored by TLC, 30% EA in Hexane), the solvent were evaporated and the crude was washed twice with diethyl ether (2×60 ml) and pentane and dried under vacuum to afford (R)-1-(benzofuran-5-yl)-N-methylpropan-2-amine hydrochloride (R-5-MAPB) (5.8 g, 81%) as off white solid. ¹HNMR(400 MHz, DMSO-d₆) δ 9.00 (bs, 2H), 7.99 (d, J=1.6 Hz, 1H), 7.57 (m, 2H), 7.21 (d, J=7.8 Hz, 1H), 6.93 (s, 1H), 3.38 (bs, 1H), 3.25 (m, 1H), 2.77 (m, 1H), 2.56 (s, 3H), 1.11 (d, J=6.28 Hz, 3H). LCMS: (ES) C₁₂H₁₅NO requires 189, found 190 [M+H]⁺. HPLC: Purity (λ 210 nm): 99.26%.

### Synthesis 21. Synthesis of (S)-1-(benzofuran-5-yl)-N-methylpropan-2-amine (S-5-MAPB)

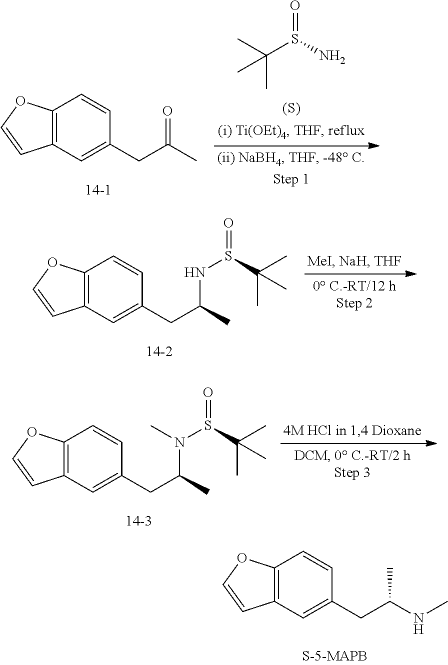

Step 1: To a stirred solution of 1-(benzofuran-5-yl)propan-2-one (14-1) (5 g, 28.70 mmol, 1 eq.) in dry THE (100 ml) was added Ti(OEt)₄ (21.06 ml, 100.45 mmol, 3.5 eq.) and (S)-2-methylpropane-2-sulfinamide (3.47 g, 28.73 mmol, 1 eq.) (dissolved in 20 ml dry THF) and the resulting reaction mixture was allowed to stir at 70° C. for 12 hrs. Upon completion (monitored by TLC, 50% EA in Hexane), the reaction mixture was cooled to 0° C., gradually to −48° C. and NaBH₄ (4.34 g, 114.81 mmol, 4 eq.) (dissolved in 20 ml dry THF) was added into the reaction mixture at −48° C. and the resulting reaction mixture was allowed to stir at −48° C. for 3 hrs. Upon completion (monitored by TLC, 50% EA in Hexane), the reaction mixture was taken to room temperature and was quenched with Methanol and Sat. NaCl solution (until white precipitate observed). The reaction mixture was then filtered through celite bed, washed with methanol (2×100 ml) and ethyl acetate (2×100 ml), evaporated under vacuum to remove the volatiles. Then the reaction mixture was extracted with ethyl acetate, washed with water, followed by brine solution. Combined organic layer was dried over anhydrous sodium sulphate, solvent was removed under vacuum to afford crude (S)-N-((S)-1-(benzofuran-5-yl)propan-2-yl)-2-methylpropane-2-sulfinamide (14-2) as yellow sticky gum (6.5 g, 81%). Crude 1H NMR (400 MHz, DMSO-d₆) δ 7.94 (d, J=7.8 Hz, 1H), 7.50 (m, 2H), 7.14 (m, 1H), 6.90 (d, J=6.36 Hz, 1H), 6.90 (d, J=6.36 Hz, 1H), 4.97 (d, J=5.96 Hz, 1H), 3.48 (m, 1H), 3.08 (m, 1H), 2.76 (m, 1H), 1.18 (m, 12H). LCMS: (ES) C₁₅H₂₁NO₂S requires 279, found 280 [M+H]⁺.

Step 2: To a stirred solution of (S)-N-((S)-1-(benzofuran-5-yl)propan-2-yl)-2-methylpropane-2-sulfinamide (14-2) (7 g, 25 mmol, 1 eq.) in dry THE (50 mL) (In a sealed tube) was added NaH (60%) (2 g, 50 mmol, 2 eq.) at 0° C. and the resulting reaction mixture was allowed to stir at 0° C. for 30 min. Then Iodomethane (3.11 ml, 50 mmol, 2 eq.) was added at 0° C. and the resulting reaction mixture was allowed to stir at room temperature for 12 h. Upon completion (monitored by TLC, 50% EA in Hexane), the reaction mixture was quenched with ice water, extracted with ethyl acetate (2×200 ml), washed with saturated ammonium chloride solution, followed by brine solution. Combined organic layer was dried over anhydrous sodium sulphate, solvent was removed under vacuum and purified by silica gel column chromatography using ethyl acetate/hexane (50:50 v/v) as eluent to afford (S)-N-((S)-1-(benzofuran-5-yl)propan-2-yl)-N,2-dimethylpropane-2-sulfinamide (14-3) as light yellow gum (4 g, 54%). 1H NMR (400 MHz, DMSO-d₆) δ 7.94 (s, 1H), 7.49 (t, J=8.4 Hz, 9.04 Hz, 2H), 7.14 (d, J=8.2, 1H), 6.89 (s, 1H), 3.55 (m, 1H), 2.92 (m, 1H), 2.88 (m, 1H), 2.51 (s, 3H), 1.27 (m, 3H), 1.07 (S, 9H). LCMS: (ES) C₁₆H₂₃NO₂S requires 293, found 294 [M+H]⁺.

Step 3: To a stirred solution of (S)-N-((S)-1-(benzofuran-5-yl)propan-2-yl)-N,2-dimethylpropane-2-sulfinamide (14-3) (7 g, 23.89 mmol, 1 eq.) in dry DCM (35 mL) was added 4M-HCl in 1,4 dioxane (70 mL) at 0° C. and the resulting reaction mixture was allowed to stir at room temperature for 2 h. Upon completion of reaction (monitored by TLC, 30% EA in Hexane), the solvent was evaporated, and the crude was washed twice with diethyl ether (2×60 ml) and pentane and dried under vacuum to afford (S)-1-(benzofuran-5-yl)-N-methylpropan-2-amine hydrochloride (S-5-MAPB) (5 g, 97%) as off white solid. ¹HNMR(400 MHz, DMSO-d₆) δ 9.06 (bs, 2H), 7.99 (d, J=1.88 Hz, 1H), 7.57 (m, 2H), 7.21 (d, J=8.28 Hz, 1H), 6.93 (d, J=1.32 Hz, 1H), 3.33 (m, 1H), 3.26 (m, 1H), 2.77 (q, 1H), 2.56 (s, 3H), 1.11 (d, J=6.4 Hz, 3H), LCMS: (ES) C₁₂H₁₅NO requires 189, found 190 [M+H]⁺. HPLC: Purity (λ 250 nm): 99.81%.

### Synthesis 22. Synthesis of (R)-1-(benzofuran-6-yl)-N-methylpropan-2-amine (R-6-MAPB)

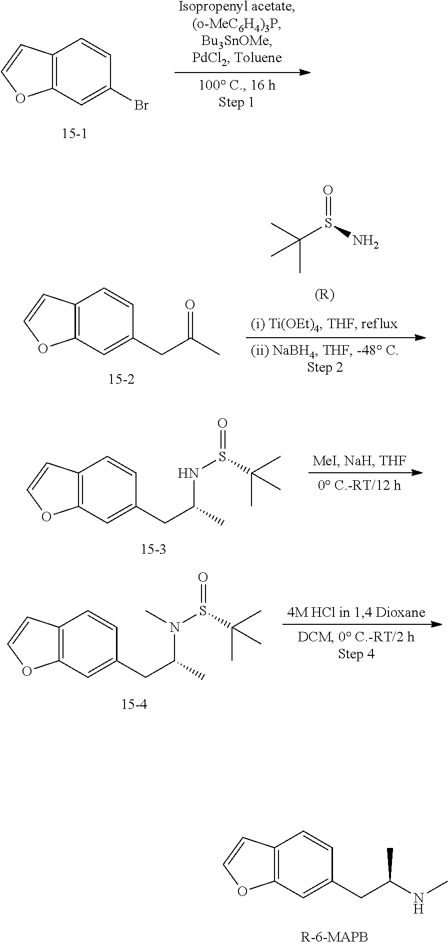

Step 1: A mixture of 6-bromobenzofuran (15-1) (10 g, 50.761 mmol), tri(o-tolyl)phosphine (0.92 g, 3.046 mmol), tributyl tin methoxide (24.4 mL, 76.14 mmol) and Isopropenyl acetate (8.49 mL, 78.17 mmol) in toluene (200 mL) was degassed under nitrogen for 15 minutes. Then palladium (II) chloride (0.63 g, 3.55 mmol) was added to this reaction mixture and continue to stir at 100° C. for 16 hours. Completion of the reaction was monitored by TLC (10% EA in Hexane). Upon completion, the reaction mixture was cooled to RT and concentrated under reduced pressure. The residue was filtered through celite bed and washed with water (100 mL) and DCM (100 mL). The reaction mixture was extracted with DCM twice (2×200 ml) and washed with water followed by brine solution. Combined organic layer was dried over anhydrous sodium sulphate, solvent was removed under vacuum and purified by silica gel column chromatography using ethyl acetate/hexane (20:80 v/v) as eluent to afford pure 1-(benzofuran-6-yl)propan-2-one (15-2) as light yellow liquid (7.0 g, 79%). 1H NMR (400 MHz, DMSO) δ 7.94 (d, J=2.0 Hz, 1H), 7.58 (d, J=7.92 Hz, 1H), 7.42 (s, 1H), 7.07 (d, J=7.84 Hz, 1H), 6.92 (d, J=1.12 Hz, 1H), 3.86 (s, 2H), 2.13 (s, 3H). LCMS: (ES) C₁₁H₁₀O₂ requires 174, found 175 [M+H]⁺.

Step 2: To a stirred solution of 1-(benzofuran-6-yl)propan-2-one (15-2) (5.5 g, 31.60 mmol) in THE (80 ml) was added Ti(OEt)₄ (23.20 mL, 110 mmol) followed by 2-methylpropane-2-sulfinamide (R)(dissolved in 5 ml THF) (3.82 g, 31.60) and the reaction mixture was allowed to stir at 70° C. for 12 h. Completion of the reaction was monitored by TLC (50% EA in Hexane). The reaction mixture was cooled to 0° C. and NaBH₄ (4.8 g, 126.4 mmol) was added to it at −45° C. and then it was allowed to stir at −45° C. for 2.5 h. Completion of the reaction was observed in TLC (50% EA in Hexane) and crude LCMS. The reaction mixture was taken to RT and then it was quenched with methanol and Saturated NaCl solution (white precipitation observed). It was filtered through celite bed, washed the celite bed with methanol and DCM then the solvent was evaporated under vacuum to remove the volatiles. Then the reaction mixture was extracted with EA twice (2×200 ml) and washed with water followed by brine solution. Combined organic layer was dried over anhydrous sodium sulphate, solvent was removed under vacuum to afford the crude (R)-N-((R)-1-(benzofuran-6-yl)propan-2-yl)-2-methylpropane-2-sulfinamide (15-3) (8.0 g), which was used for next step without further purification. 1H NMR (400 MHz, DMSO) δ 7.92 (d, J=1.96 Hz, 1H), 7.56 (d, J=7.84 Hz, 1H), 7.44 (s, 1H), 7.11 (d, J=8.08 Hz, 1H), 6.90 (d, J=1.04 Hz, 1H), 4.98 (d, J=6.0 Hz, 1H), 3.49 (m, 1H), 3.08 (m, 1H), 2.79 (m, 1H), 1.08 (m, 12H). LCMS: (ES) C15H21NO2S, requires 279, found 280 [M+H]⁺.

Step 3: To a stirred solution of crude (R)-N-((R)-1-(benzofuran-6-yl)propan-2-yl)-2-methylpropane-2-sulfinamide (15-3) (8.0 g, 28.67 mmol) in THE (100 mL), NaH (60%) (2.2 g, 57.34 mmol) at 0° C. was added portion-wise then the reaction mixture was stirred at 0° C. for 30 min after that Iodomethane (3.54 mL, 57.34 mmol) was added to it and the reaction mixture was stirred at RT for 12 h. Completion of the reaction was monitored by TLC (20% EA in Hexane). Upon completion, the reaction mixture was diluted with cold water (100 mL) extracted with EA twice (2×200 ml) and organic layer was washed with NaHCO₃ solution (100 mL) followed by brine solution. Combined organic layer was dried over anhydrous sodium sulphate, solvent was removed under vacuum and purified by silica gel column chromatography using 15-20% ethyl acetate hexane to afford pure (R)-N-((R)-1-(benzofuran-6-yl)propan-2-yl)-N,2-dimethylpropane-2-sulfinamide (15-4) (4.0 g, 47%) as a colorless sticky solid. 1H NMR (400 MHz, DMSO) δ 7.91 (d, J=2.04 Hz, 1H), 7.56 (d, J=7.92 Hz, 1H), 7.42 (s, 1H), 7.10 (d, J=8.04 Hz, 1H), 6.90 (d, J=1.36 Hz, 1H), 3.59 (m, 1H), 2.95 (dd, J=13.42 Hz 1H), 2.84 (dd, J=13.38 Hz 1H), 2.51 (s, 3H), 1.10 (d, J=6.68 Hz, 3H), 1.02 (S, 9H). LCMS: (ES) C16H23NO2S, requires 293, found 294 [M+H]⁺.

Step 4: To a stirred solution of (R)-N-((R)-1-(benzofuran-6-yl)propan-2-yl)-N,2-dimethylpropane-2-sulfinamide (15-4) (9.4 g, 32.03 mmol) in 1, 4 dioxane (60 mL) was added 4(M) HCl in 1, 4 dioxane (30.0 mL) at 0° C. and the resulting reaction mixture was allowed to stir at room temperature for 5 h. Upon completion of reaction (monitored by TLC, 10% EA in Hexane), the solvent were evaporated and the residue was dissolved in methanol and diethyl ether was added to it for precipitation, finally filter to get pure (R)-1-(benzofuran-6-yl)-N-methylpropan-2-amine hydrochloride (R-6-MAPB) (6.1 g, 84%) as white solid. 1H NMR (400 MHz, DMSO) δ 9.00 (bs, 2H), 7.96 (d, J=2.08 Hz, 1H), 7.62 (d, J=7.92 Hz, 1H), 7.53 (s, 1H), 7.16 (d, J=7.52 Hz, 1H), 6.93 (d, J=1.48 Hz, 1H), 3.41 (bs, 1H), 3.30 (dd, J=13.28 Hz, 1H), 2.80 (dd, J=13.2 Hz, 1H), 2.56 (s, 3H), 1.12 (d, J=6.48 Hz, 3H). LCMS: (ES) C12H16ClNO, requires 189, found 190 [M+H]⁺. HPLC: Purity (λ 250 nm): 99.58%.

### Synthesis 23. Synthesis of (S)-1-(benzofuran-6-yl)-N-methylpropan-2-amine (S-6-MAPB)

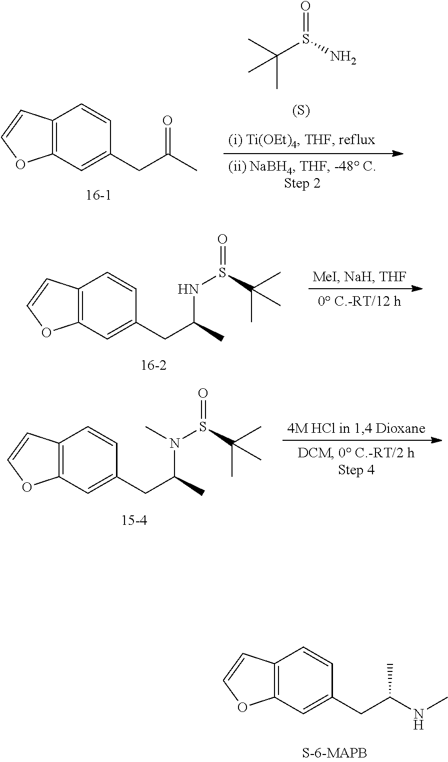

Step 1: To a stirred solution of 1-(benzofuran-6-yl)propan-2-one (16-1) (5 g, 28.70 mmol, 1 eq.) in dry THF (100 mL) was added Ti(OEt)₄ (21.06 mL, 100.45 mmol, 3.5 eq.) and (S)-2-methylpropane-2-sulfinamide (3.47 g, 28.73 mmol, 1 eq.) (dissolved in 20 mL dry THF) and the resulting reaction mixture was allowed to stir at 70° C. for 12 h. Upon completion, monitored by TLC (50% EA in Hexane), the reaction mixture was cooled to 0° C., gradually to −48° C. and NaBH₄ (4.34 g, 114.81 mmol, 4 eq.) (dissolved in 20 mL dry THF) was added into the reaction mixture at −48° C. and the resulting reaction mixture was allowed to stir at −48° C. for 3 h. Upon completion, monitored by TLC (50% EA in Hexane), the reaction mixture was taken to room temperature and was quenched with Methanol and saturated NaCl solution (until white precipitate observed). The reaction mixture was then filtered through celite bed, washed the celite bed with methanol (2×100 ml) and ethyl acetate (2×100 mL), and evaporated under vacuum to remove the volatiles. Then the reaction mixture was extracted with ethyl acetate, washed with water, followed by brine solution. Combined organic layer was dried over anhydrous sodium sulphate, solvent was removed under vacuum to afford crude (S)-N-((S)-1-(benzofuran-6-yl)propan-2-yl)-2-methylpropane-2-sulfinamide (16-2) as yellow sticky gum (7.5 g, 93%). Crude 1H NMR (400 MHz, DMSO-d₆) δ 7.92 (d, J=2.08 Hz 1H), 7.56 (d, J=7.92 Hz, 1H), 7.44 (s, 1H), 7.11 (d, J=7.96 Hz, 1H), 6.90 (d, J=1.84 Hz, 1H), 4.96 (d, J=6.08 Hz, 1H), 3.30 (m, 1H), 3.08 (m, 1H), 2.80 (m, 1H), 1.10 (m, 9H), 1.08 (m, 3H). LCMS: (ES) C₁₅H₂₁NO₂S, requires 279, found 280 [M+H]⁺.

Step 2: To a stirred solution of (S)-N-((S)-1-(benzofuran-6-yl)propan-2-yl)-2-methylpropane-2-sulfinamide (16-2) (8 g, 28.67 mmol, 1 eq.) in dry THF (60 mL) (In a sealed tube) was added NaH (60%) (2.28 g, 57.26 mmol, 2 eq.) at 0° C. and the resulting reaction mixture was allowed to stir at 0° C. for 30 min. Then Iodomethane (3.56 mL, 57.26 mmol, 2 eq.) was added at 0° C. and the resulting reaction mixture was allowed to stir at room temperature for 12 h. Upon completion, monitored by TLC (50% EA in Hexane), the reaction mixture was quenched with ice water, extracted with ethyl acetate (2×200 ml), washed with saturated ammonium chloride solution followed by brine solution. Combined organic layer was dried over anhydrous sodium sulphate, solvent was removed under vacuum and purified by silica gel column chromatography using ethyl acetate/hexane (50:50 v/v) as eluent to afford (S)-N-((S)-1-(benzofuran-6-yl)propan-2-yl)-N,2-dimethylpropane-2-sulfinamide (16-3) as light yellow gum (4.5 g, 53%). 1H NMR (400 MHz, DMSO-d₆) δ 7.92 (d, J=1.84 Hz, 1H), 7.56 (d, J=7.84 Hz, 1H), 7.42 (s, 1H), 7.10 (d, J=7.96 Hz, 1H), 6.90 (S, 1H), 3.57 (d, J=7.32 Hz, 1H), 2.92 (m, 1H), 2.84 (m, 1H), 2.51 (s, 3H), 1.10 (d, J=6.6 Hz, 3H), 1.02 (s, 9H). LCMS: (ES) C₁₆H₂₃NO₂S, requires 293, found 294 [M+H]⁺.

Step 3: To a stirred solution of (S)-N-((S)-1-(benzofuran-6-yl)propan-2-yl)-N,2-dimethylpropane-2-sulfinamide (16-3) (5.4 g, 18.40 mmol, 1 eq.) in dry DCM (45 mL) was added 4(M) HCl in 1,4 dioxane (90 mL) at 0° C. and the resulting reaction mixture was allowed to stir at room temperature for 2 h. Upon completion of reaction (monitored by TLC, 30% EA in Hexane), the solvent was evaporated, and the crude was washed twice with diethyl ether (2×100 ml) and pentane and dried under vacuum to afford (S)-1-(benzofuran-6-yl)-N-methylpropan-2-amine hydrochloride S-6-MAPB (3.5 g, 84%) as white solid. ¹HNMR(400 MHz, DMSO-d₆) δ 9.01 (bs, 2H), 7.96 (d, J=2.04 Hz, 1H), 7.62 (d, J=7.88 Hz, 1H), 7.53 (s, 1H), 7.16 (d, J=7.88 Hz, 1H), 6.93 (d, J=1.64 Hz, 1H), 3.44 (bs, 1H), 3.30 (q, 1H), 2.80 (m, 1H), 2.56 (s, 3H), 1.12 (d, J=6.48 Hz, 3H). LCMS: (ES) C₁₂H₁₅NO, requires 189, found 190 [M+H]⁺. HPLC:Purity (λ 200 nm): 99.61%.

### Additional Synthetic Procedures

### Synthesis of 1-(5-Fluoro-1H-indol-3-yl)-2-(methylamino) propan-1-one hydrochloride (BK-5F-NM-AMT)

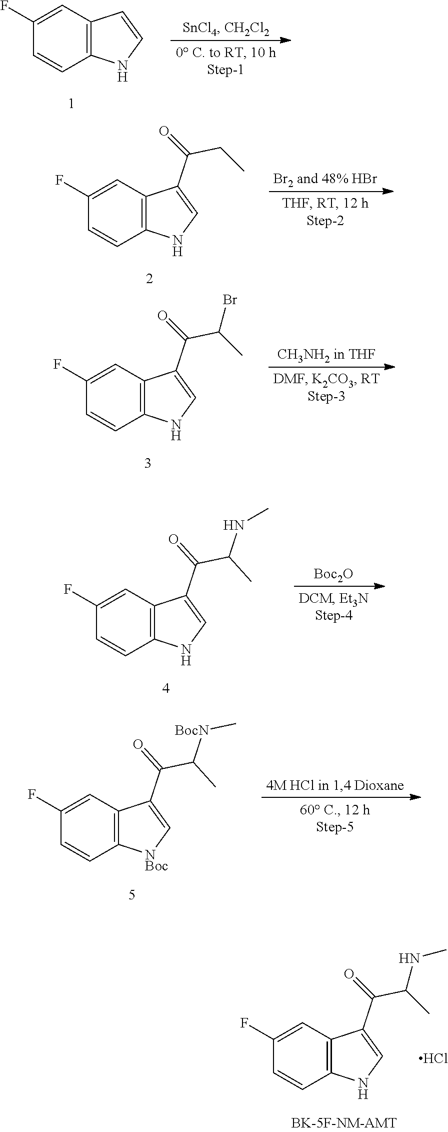

### Step 1: Synthesis of 1-(5-Fluoro-1H-indol-3-yl) propan-1-one (2)

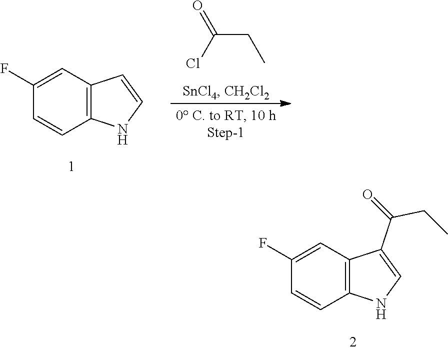

To a stirred solution of 5-Fluoro-1H-indole (1) (10 g, 73.99 mmol, 1 eq.) in dry DCM (200 mL) was added SnCl₄ (10.39 mL, 88.79 mmol, 1.2 eq.) at 0° C. under argon atmosphere. The resulting reaction mixture was allowed to stir at 0° C. for 30 min, then continue to stir at room temperature and Propionyl chloride (6.46 mL, 73.99 mmol, 1 eq.) and Nitromethane (140 mL) were added to the reaction mixture and continue to stir at room temperature for 10 h. Upon completion, monitored by TLC (30% EA in Hexane), the reaction mixture was quenched with water, extracted with ethyl acetate (2×200 mL), washed with water, followed by brine solution. Combined organic layer was dried over anhydrous sodium sulphate. Solvent was removed under vacuum and purified by silica gel column chromatography using ethyl acetate/hexane (20:80 v/v) as eluent to afford 1-(5-Fluoro-1H-indol-3-yl)propan-1-one (2) as light yellow solid (6 g, 42.41%). ¹H NMR (400 MHz, DMSO-d₆) δ 11.99 (s, 1H), 8.37 (d, J=2.64 Hz, 1H), 7.86-7.83 (dd, J=2.52 Hz, 10.08 Hz, 1H), 7.48-7.45 (dd, J=4.64 Hz, 8.84 Hz, 1H), 7.08-7.02 (m, 1H), 2.89-2.83 (q, 2H), 1.12-1.08 (t, J=7.36 Hz, 7.44 Hz, 3H). MS (ES) C₁₁H₁₀FNO requires 191, found 192 [M+H]⁺.

### Step 2: Synthesis of 2-bromo-1-(5-Fluoro-1H-indol-3-yl) propan-1-one (3)

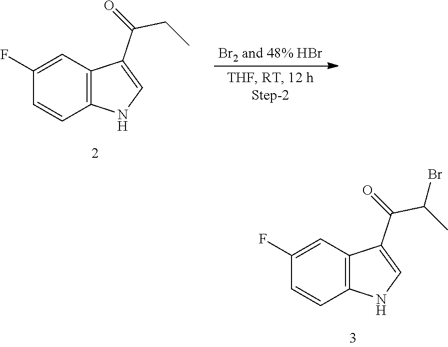

To a stirred solution of 1-(5-Fluoro-1H-indol-3-yl)propan-1-one (2) (3.5 g, 18.31 mmol, 1 eq.) in dry THF (50 mL) was added Hydrobromic acid 48% in Water (31.83 mL, 586.17 mmol, 32 eq.) and Bromine (1.03 mL, 20.15 mmol, 1.1 eq.) at 0° C. and the resulting reaction mixture was allowed to stir at room temperature for 12 h. Upon completion, monitored by TLC (20% EA in Hexane), the reaction mixture was basified with saturated sodium carbonate solution up to pH-8 and was extracted with ethyl acetate (2×150 mL), washed with water, followed by brine solution. Combined organic layer was dried over anhydrous sodium sulphate, solvent was removed under vacuum and purified by silica gel column chromatography using ethyl acetate/hexane (10:90 v/v) as eluent to afford 2-bromo-1-(5-Fluoro-1H-indol-3-yl)propan-1-one (3) as light yellow solid (2.2 g, 44.65%). ¹H NMR (400 MHz, DMSO-d₆) δ 12.27 (s, 1H), 8.58 (d, J=3.08 Hz, 1H), 7.86-7.82 (dd, J=2.56 Hz, 9.84 Hz, 1H), 7.53-7.50 (q, 1H), 7.13-7.08 (m, 1H), 5.65-5.60 (q, 1H), 1.77 (d, J=6.56 Hz, 3H). MS (ES) C₁₁H₉BrFNO requires 269, found 272 [M+H]⁺.

### Step 3: Synthesis of 1-(5-Fluoro-1H-indol-3-yl)-2-(methylamino) propan-1-one (4)

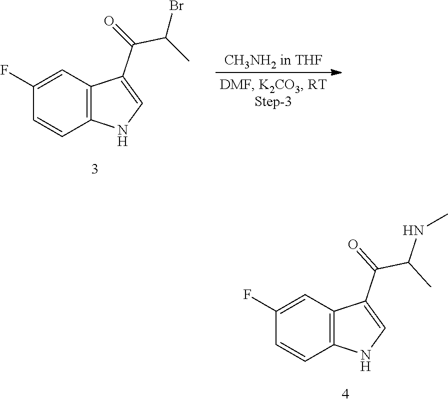 [SMILES: CC(Br)C(=O)c1c[nH]c2ccc(F)cc12 ; CNC(C)C(=O)c1c[nH]c2ccc(F)cc12]

To a stirred solution of 2-bromo-1-(5-Fluoro-1H-indol-3-yl)propan-1-one (3) (4.4 g, 16.35 mmol, 1 eq.) in dry DMF (50 mL) was added potassium carbonate (3.39 g, 24.53 mmol, 1.5 eq.) and methyl amine 2M in THF (49 mL, 98.14 mmol, 6 eq.) and the resulting reaction mixture was allowed to stir at room temperature for 12 h. Upon completion, monitored by TLC (10% EA in Hexane), the volatiles were evaporated and the reaction mixture was extracted with ethyl acetate (2×100 mL), washed with cold water (twice), followed by brine solution. Combined organic layer was dried over anhydrous sodium sulphate, solvent was removed under vacuum to afford crude 1-(5-Fluoro-1H-indol-3-yl)-2-(methylamino) propan-1-one (4) as yellow sticky solid (2.3 g, 63.84%). ¹H NMR crude data (400 MHz, DMSO-d₆) δ 12.12 (bs, 1H), 8.54 (s, 1H), 7.90 (d, J=9.72 Hz, 1H), 7.49-7.46 (m, 1H), 7.09-7.05 (m, 1H), 3.98-3.93 (m, 1H), 2.21 (s, 3H), 1.18 (d, J=6.72 Hz, 3H), MS (ES) C₁₂H₁₃FN₂O requires 220, found 221 [M+H]⁺.

### Step 4: Synthesis of tert-butyl 3-(N-(tert-butoxycarbonyl)-N-methylalanyl)-5-fluoro-1H-indole-1-carboxylate (5)

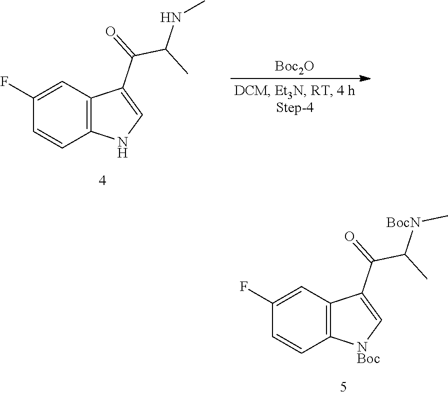 [SMILES: "Boc2O" ; CNC(C)C(=O)c1c[nH]c2ccc(F)cc12 ; CC(C(=O)c1cn(C(=O)OC(C)(C)C)c2ccc(F)cc12)N(C)C(=O)OC(C)(C)C]

To a stirred solution of crude 1-(5-fluoro-1H-indol-3-yl)-2-(methylamino)propan-1-one (4) (2.3 g, 9.74 mmol, 1 eq.) in dry DCM (40 mL) was added triethylamine (2.71 mL, 19.49 mmol, 2 eq.) and Boc anhydride (5.59 mL, 24.34 mmol, 2.5 eq.) and the resulting reaction mixture was allowed to stir at room temperature for 4 h. Upon completion (monitored by TLC, 10% EA in Hexane), the reaction mixture was extracted with DCM (2×100 mL), washed with water, followed by brine solution. Combined organic layer was dried over anhydrous sodium sulphate, solvent was evaporated under vacuum and purified by silica gel column chromatography using ethyl acetate/hexane (10:90 v/v) as eluent to afford tert-butyl 3-(N-(tert-butoxycarbonyl)-N-methylalanyl)-5-fluoro-1H-indole-1-carboxylate (5) as yellow sticky gum (3.0 g, 73%). ¹H NMR (400 MHz, DMSO-d₆) δ 8.65-8.47 (s, 1H), 8.10 (bs, 1H), 7.92-7.90 (m, 1H), 7.32-7.30 (m, 1H), 5.44-5.08 (s, 1H), 2.87 (m, 1H), 1.65 (s, 9H), 1.40-1.25 (m, 12H). MS (ES) C₂₂H₂₉FN₂O₅ requires 420, found 421 [M+H]⁺, 321 [M−100+2], rotamers observed.

### Step 5: Synthesis of 1-(5-Fluoro-1H-indol-3-yl)-2-(methylamino) propan-1-one hydrochloride (BK-5F-NM-AMT)

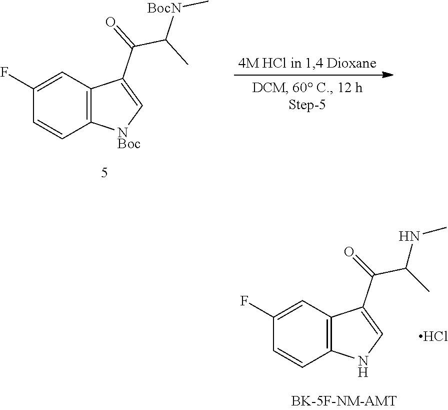 [SMILES: CC(C(=O)c1cn(C(=O)OC(C)(C)C)c2ccc(F)cc12)N(C)C(=O)OC(C)(C)C ; "4M HCl in 1,4 Dioxane" ; CNC(C)C(=O)c1c[nH]c2ccc(F)cc12 ; Cl]

To a stirred solution of tert-butyl 3-(N-(tert-butoxycarbonyl)-N-methylalanyl)-5-fluoro-1H-indole-1-carboxylate (5) (3.0 g, 9.36 mmol, 1 eq.) in dry DCM (40 mL) was added 4M HCl in 1,4 dioxane (30 mL) at 0° C. and the resulting reaction mixture was allowed to stir at 60° C. for 12 h. Upon completion of reaction (monitored by TLC, 10% EA in Hexane), the solvent were evaporated and the crude was washed twice with diethyl ether (2×50 mL) and pentane (1×50 mL) then dried under vacuum to afford 1-(5-fluoro-1H-indol-3-yl)-2-(methylamino)propan-1-one hydrochloride (BK-5F-NM-AMT) as off white solid (1.5 g, 72.7%). ¹H NMR (400 MHz, DMSO-d₆) δ 12.77 (s, 1H), 9.42 (bs, 2H), 8.64 (s, 1H), 7.85 (d, J=9.36 Hz, 1H), 7.57-7.54 (m, 1H), 7.14-7.10 (t, J=8.28 Hz, 8.76 Hz, 1 Hz), 4.86 (d, J=6.84 Hz, 1H), 2.54 (s, 3H), 1.53 (d, J=6.68 Hz, 3H). MS (ES) C₁₂H₁₃FN₂O requires 220, found 221 [M+H]⁺. HPLC: Purity (λ 210 nm): 99.83%.

### Synthesis of 1-(5-chloro-1H-indol-3-yl)-2-(methylamino) propan-1-one hydrochloride (BK-5Cl-NM-AMT)

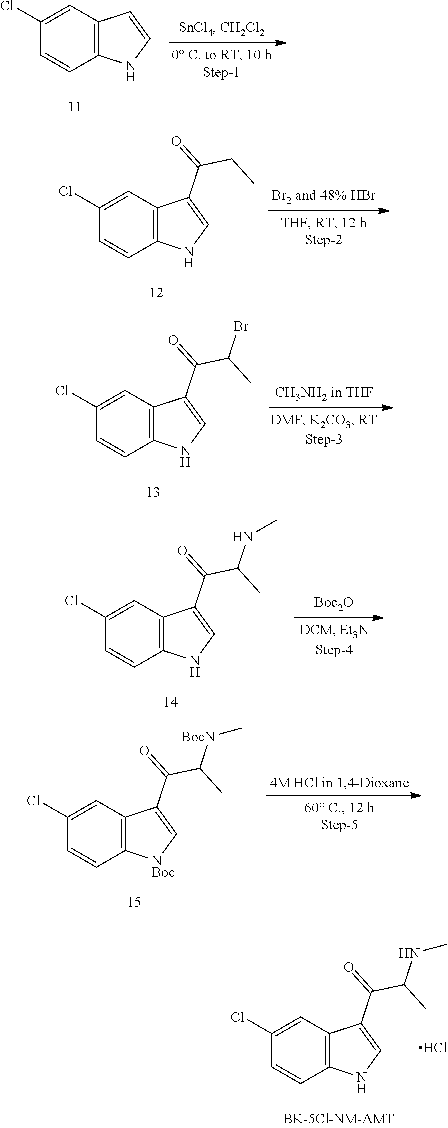

### Step 1: Synthesis of 1-(5-chloro-1H-indol-3-yl) propan-1-one (12)

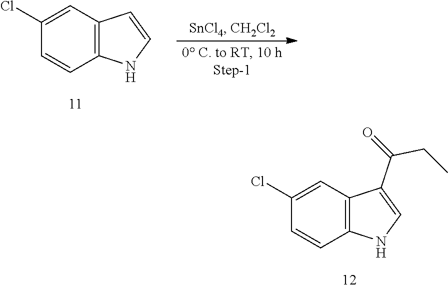

To a stirred solution of 5-chloro-1H-indole (11) (10 g, 65.96 mmol, 1 eq.) in dry DCM (200 mL) was added SnCl₄ (9.26 mL, 79.16 mmol, 1.2 eq.) at 0° C. under argon atmosphere. The resulting reaction mixture was allowed to stir at 0° C. for 30 min, then continue to stir at room temperature and Propionyl chloride (5.26 mL, 65.96 mmol, 1 eq.) and Nitromethane (140 mL) were added to the reaction mixture and continue to stir at room temperature for 10 h. Upon completion, monitored by TLC (30% EA in Hexane), the reaction mixture was quenched with water, extracted with ethyl acetate (2×200 mL), washed with water, followed by brine solution. Combined organic layer was dried over anhydrous sodium sulphate. Solvent was removed under vacuum and purified by silica gel column chromatography using ethyl acetate/hexane (20:80 v/v) as eluent to afford 1-(5-chloro-1H-indol-3-yl)propan-1-one (12) as light yellow solid (6 g, 44%). ¹H NMR (400 MHz, DMSO-d₆) δ 12.06 (s, 1H), 8.38 (d, J=2.88 Hz, 1H), 8.17 (d, J=1.56 Hz, 1H), 7.49 (d, J=8.6 Hz, 1H), 7.22-7.20 (dd, J=1.84 Hz, 8.6 Hz, 1H), 2.89-2.84 (q, 2H), 1.12-1.08 (t, J=7.36 Hz, 7.40 Hz, 3H). MS (ES) C₁₁H₁₀ClNO requires 207, found 208 [M+H]⁺.

### Step 2: Synthesis of 2-bromo-1-(5-chloro-1H-indol-3-yl) propan-1-one (13)

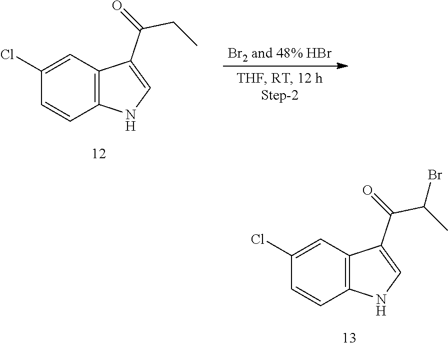

To a stirred solution of 1-(5-chloro-1H-indol-3-yl)propan-1-one (12) (3.5 g, 16.85 mmol, 1 eq.) in dry THF (50 mL) was added Hydrobromic Acid 48% in Water (29.28 mL, 539.369 mmol, 32 eq.) and Bromine (0.95 mL, 18.54 mmol, 1.1 eq.) at 0° C. and the resulting reaction mixture was allowed to stir at room temperature for 12 h. Upon completion, monitored by TLC (20% EA in Hexane), the reaction mixture was basified with saturated Sodium Carbonate solution up to pH-8 and was extracted with ethyl acetate (2×150 mL), washed with water, followed by brine solution. Combined organic layer was dried over anhydrous sodium sulphate, solvent was removed under vacuum and purified by silica gel column chromatography using ethyl acetate/hexane (10:90 v/v) as eluent to afford 2-bromo-1-(5-chloro-1H-indol-3-yl)propan-1-one (13) as light yellow solid (2.2 g, 45.5%). ¹H NMR (400 MHz, DMSO-d₆) δ 12.32 (s, 1H), 8.58 (d, J=3.2 Hz, 1H), 8.15 (d, J=2 Hz, 1H), 7.54 (d, J=8.64 Hz, 1H), 7.28 (dd, J=2.16 Hz, 8.68 Hz, 1H), 5.65-5.60 (q, 1H), 1.69 (d, J=6.92 Hz, 3H). MS (ES) C₁₁H₉BrClNO requires 285, found 286 [M+H]⁺.

### Step 3: Synthesis of 1-(5-chloro-1H-indol-3-yl)-2-(methylamino) propan-1-one (14)

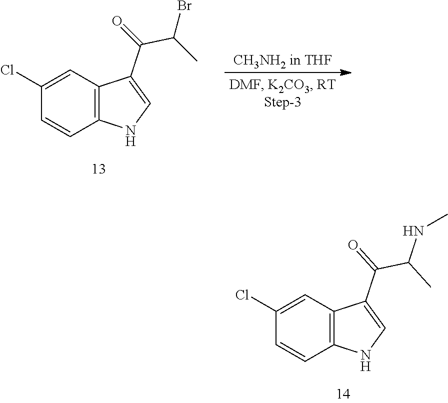 [SMILES: CC(Br)C(=O)c1c[nH]c2ccc(Cl)cc12 ; CNC(C)C(=O)c1c[nH]c2ccc(Cl)cc12]

To a stirred solution of 2-bromo-1-(5-chloro-1H-indol-3-yl)propan-1-one (13) (2.1 g, 7.32 mmol, 1 eq.) in dry DMF (30 mL) was added Potassium Carbonate (1.52 g, 10.99 mmol, 1.5 eq.) and methyl amine 2M in THE (22 mL, 43.97 mmol, 6 eq.), then the resulting reaction mixture was allowed to stir at room temperature for 12 h. Upon completion, monitored by TLC (10% EA in Hexane), the volatiles were evaporated and the reaction mixture was extracted with ethyl acetate (2×100 mL), washed with cold water (twice), followed by brine solution. Combined organic layer was dried over anhydrous sodium sulphate, solvent was removed under vacuum to afford crude 1-(5-chloro-1H-indol-3-yl)-2-(methylamino) propan-1-one (14) as yellow sticky solid (1.7 g, 98%). Proceeded for next step without further purification.

### Step 4: Synthesis of tert-butyl 3-(N-(tert-butoxycarbonyl)-N-methylalanyl)-5-chloro-1H-indole-1-carboxylate (15)

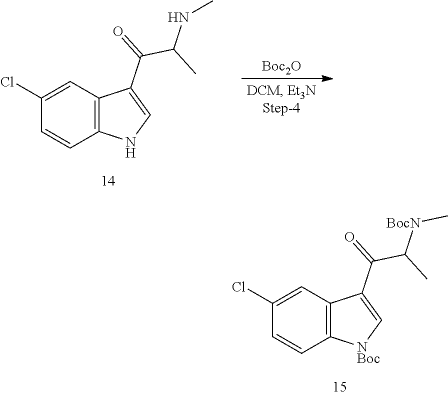 [SMILES: "Boc2O" ; CNC(C)C(=O)c1c[nH]c2ccc(Cl)cc12 ; CC(C(=O)c1cn(C(=O)OC(C)(C)C)c2ccc(Cl)cc12)N(C)C(=O)OC(C)(C)C]

To a stirred solution of crude 1-(5-chloro-1H-indol-3-yl)-2-(methylamino)propan-1-one (14) (1.8 g, 7.62 mmol, 1 eq.) in dry DCM (30 mL) was added triethylamine (2.12 mL, 15.25 mmol, 2 eq.) and Boc anhydride (3.5 mL, 15.25 mmol, 2 eq.), then the resulting reaction mixture was allowed to stir at room temperature for 4 h. Upon completion (monitored by TLC, 10% EA in Hexane), the reaction mixture was extracted with DCM (2×100 mL), washed with water, followed by brine solution. Combined organic layer was dried over anhydrous sodium sulphate, solvent was evaporated under vacuum and purified by silica gel column chromatography using ethyl acetate/hexane (10:90 v/v) as eluent to afford tert-butyl 3-(N-(tert-butoxycarbonyl)-N-methylalanyl)-5-chloro-1H-indole-1-carboxylate (15) as yellow sticky gum (1.3 g, 50.6%). ¹H NMR (400 MHz, DMSO-d₆) δ 8.63-8.45 (s, 1H), 8.20 (d, J=2 Hz, 1H), 8.11-8.09 (d, J=8.28 Hz, 1H), 7.48-7.45 (dd, J=1.48 Hz, 8.80 Hz, 1H), 5.43-5.08 (m, 1H), 2.83-2.66 (s, 3H), 1.65 (s, 9H), 1.40-1.26 (s, 12H). MS (ES) C₂₂H₂₉C₁N205 requires 436, found 436.8, 335 [M−100+2], rotamer observed.

### Step 5: Synthesis of 1-(5-chloro-1H-indol-3-yl)-2-(methylamino) propan-1-one hydrochloride (BK-5Cl-NM-AMT)

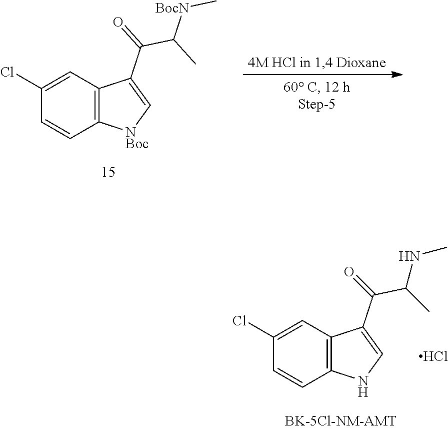 [SMILES: CC(C(=O)c1cn(C(=O)OC(C)(C)C)c2ccc(Cl)cc12)N(C)C(=O)OC(C)(C)C ; "4M HCl in 1,4 Dioxane" ; CNC(C)C(=O)c1c[nH]c2ccc(Cl)cc12 ; Cl]

To a stirred solution of tert-butyl 3-(N-(tert-butoxycarbonyl)-N-methylalanyl)-5-chloro-1H-indole-1-carboxylate (15) (2.2 g, 6.53 mmol, 1 eq.) in dry DCM (20 mL) was added 4M HCl in 1,4 dioxane (30 mL) at 0° C. and the resulting reaction mixture was allowed to stir at 60° C. for 12 h. Upon completion of reaction (monitored by TLC, 10% EA in Hexane), the solvent were evaporated and the crude was washed twice with diethyl ether (2×50 mL) and pentane (1×50 mL), then dried under vacuum to afford 1-(5-chloro-1H-indol-3-yl)-2-(methylamino)propan-1-one hydrochloride (BK-5Cl-NM-AMT) as off white solid (1.3 g, 72.8%). ¹H NMR (400 MHz, DMSO-d₆) δ 12.68 (s, 1H), 9.22 (bs, 1H), 8.63 (s, 1H), 8.15 (d, J=1.76 Hz, 1H), 7.58 (d, J=8.64 Hz, 1H), 7.32-7.29 (dd, J=1.84 Hz, 8.64 Hz, 1 Hz), 4.86-4,81 (q, 1H), 2.56 (s, 3H), 1.52 (d, J=6.96 Hz, 3H). MS (ES) C₁₂H₁₃ClN₂O requires 236, found 237 [M+H]⁺. HPLC: Purity (λ 220 nm): 99.43%.

### Synthesis of 1-(5-bromo-1H-indol-3-yl)-2-(methylamino) propan-1-one hydrochloride (BK-5Br-NM-AMT)

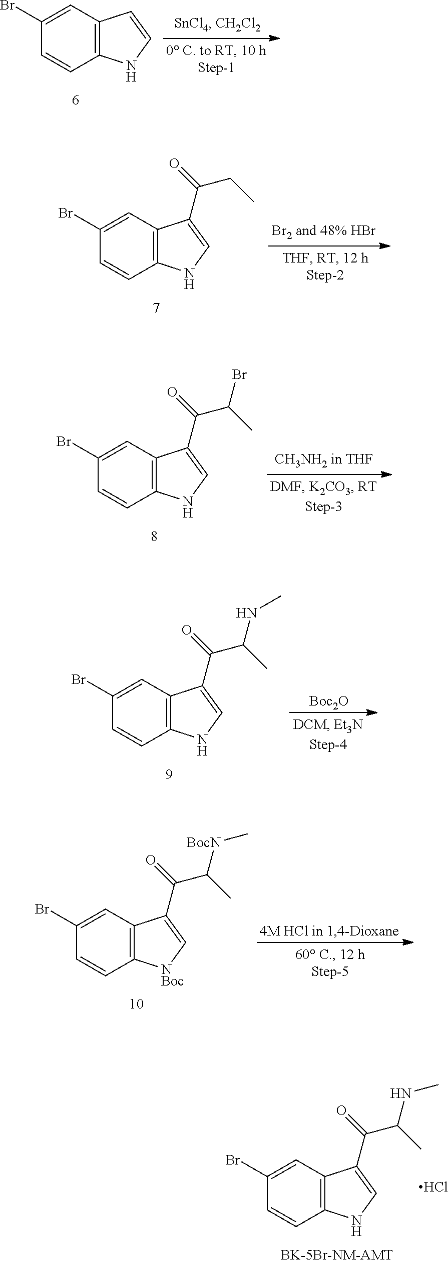

### Step 1: Synthesis of 1-(5-bromo-1H-indol-3-yl) propan-1-one (7)

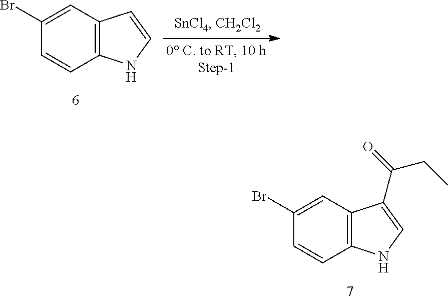

To a stirred solution of 5-bromo-1H-indole (6) (10 g, 51.02 mmol, 1 eq.) in dry DCM (200 mL) was added SnCl₄ (7.18 mL, 61.22 mmol, 1.2 eq.) at 0° C. under argon atmosphere. The resulting reaction mixture was allowed to stir at 0° C. for 30 min, then continue to stir at room temperature and Propionyl chloride (4.49 mL, 51.02 mmol, 1 eq.) and Nitromethane (140 mL) were added to the reaction mixture and the resulting reaction mixture was allowed to stir at room temperature for 10 h. Upon completion, monitored by TLC (30% EA in Hexane), the reaction mixture was quenched with water, extracted with ethyl acetate (2×200 mL), washed with water, followed by brine solution. Combined organic layer was dried over anhydrous sodium sulphate. Solvent was removed under vacuum and purified by silica gel column chromatography using ethyl acetate/hexane (20:80 v/v) as eluent to afford 1-(5-bromo-1H-indol-3-yl)propan-1-one (7) as light yellow solid (5 g, 39%). ¹H NMR (400 MHz, DMSO-d₆) δ 12.07 (s, 1H), 8.36 (s, 1H), 8.32 (d, J=1.64 Hz, 1H), 7.45 (d, J=8.6 Hz, 1H), 7.34-7.31 (dd, J=1.84 Hz, 8.64 Hz, 1H), 2.89-2.84 (q, 2H), 1.12-1.08 (t, J=7.36 Hz, 3H). MS (ES) C₁₁H₁₀BrNO requires 251, found 252 [M+H]⁺.

### Step 2: Synthesis of 2-bromo-1-(5-bromo-1H-indol-3-yl) propan-1-one (8)

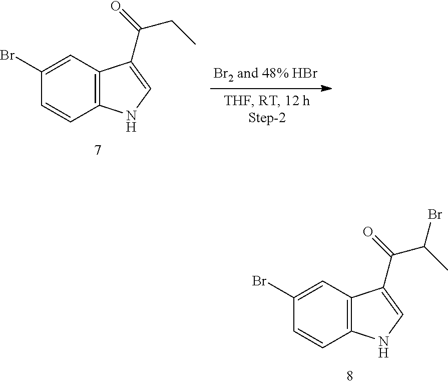

To a stirred solution of 1-(5-bromo-1H-indol-3-yl)propan-1-one (7) (5 g, 19.84 mmol, 1 eq.) in dry THE (50 mL) was added Hydrobromic Acid 48% in Water (51.37 mL, 634.92 mmol, 32 eq.) and Bromine (1.11 mL, 21.82 mmol, 1.1 eq.) at 0° C. and the resulting reaction mixture was allowed to stir at room temperature for 12 h. Upon completion, monitored by TLC (20% EA in Hexane), the reaction mixture was basified with saturated Sodium Carbonate solution up to pH-8 and was extracted with ethyl acetate (2×150 mL), washed with water, followed by brine solution. Combined organic layer was dried over anhydrous sodium sulphate, solvent was removed under vacuum and purified by silica gel column chromatography using ethyl acetate/hexane (10:90 v/v) as eluent to afford 2-bromo-1-(5-bromo-1H-indol-3-yl)propan-1-one (8) as light yellow solid (4 g, 60%). ¹H NMR (400 MHz, DMSO-d₆) δ 12.33 (s, 1H), 8.56 (d, J=3 Hz, 1H), 8.31 (d, J=1.4 Hz, 1H), 7.49 (d, J=8.64 Hz, 1H), 7.39-7.37 (dd, J=1.68 Hz, 8.60 Hz, 1H), 5.65-5.60 (q, 1H), 1.77 (d, J=6.6 Hz, 3H). MS (ES) C₁₁H₉Br₂NO requires 329, found 330 [M+H]⁺.

### Step 3: Synthesis of 1-(5-bromo-1H-indol-3-yl)-2-(methylamino)propan-1-one (9)

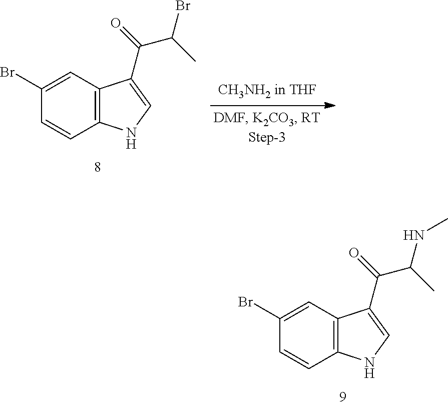 [SMILES: CC(Br)C(=O)c1c[nH]c2ccc(Br)cc12 ; CNC(C)C(=O)c1c[nH]c2ccc(Br)cc12]

To a stirred solution of 2-bromo-1-(5-bromo-1H-indol-3-yl)propan-1-one (8) (4 g, 12.08 mmol, 1 eq.) in dry DMF (40 mL) was added Potassium Carbonate (2.5 g, 18.12 mmol, 1.5 eq.) and methyl amine 2M in THE (36.25 mL, 72.50 mmol, 6 eq.) and the resulting reaction mixture was allowed to stir at room temperature for 12 h. Upon completion, monitored by TLC (10% EA in Hexane), the volatiles were evaporated and the reaction mixture was extracted with ethyl acetate (2×100 mL), washed with cold water (twice), followed by brine solution. Combined organic layer was dried over anhydrous sodium sulphate, solvent was removed under vacuum to afford crude 1-(5-bromo-1H-indol-3-yl)-2-(methylamino) propan-1-one (9) as yellow sticky solid (3.2 g, 94%). ¹H NMR crude data (400 MHz, DMSO-d₆) δ 8.54 (s, 1H), 8.36 (s, 1H), 7.46 (d, J=8.52 Hz, 1H), 7.35 (d, J=8.56 Hz, 1H), 3.98-3.93 (q, 1H), 2.20 (s, 3H), 1.18 (d, J=6.76 Hz, 3H). MS (ES) C₁₂H₁₃BrN₂O requires 280, found 281 [M+H]⁺.

### Step 4: Synthesis of tert-butyl 5-bromo-3-(N-(tert-butoxycarbonyl)-N-methylalanyl)-1H-indole-1-carboxylate (10)

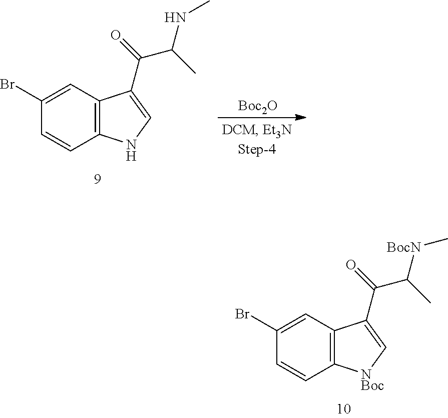 [SMILES: "Boc2O" ; CNC(C)C(=O)c1c[nH]c2ccc(Br)cc12 ; CC(C(=O)c1cn(C(=O)OC(C)(C)C)c2ccc(Br)cc12)N(C)C(=O)OC(C)(C)C]

To a stirred solution of crude 1-(5-bromo-1H-indol-3-yl)-2-(methylamino)propan-1-one (9) (3.2 g, 11.38 mmol, 1 eq.) in dry DCM (30 mL) was added triethylamine (3.28 mL, 22.77 mmol, 2 eq.) and Boc anhydride (5.22 mL, 22.77 mmol, 2 eq.) and the resulting reaction mixture was allowed to stir at room temperature for 4 h. Upon completion (monitored by TLC, 10% EA in Hexane), the reaction mixture was extracted with DCM (2×100 mL), washed with water, followed by brine solution. Combined organic layer was dried over anhydrous sodium sulphate, solvent was evaporated under vacuum and purified by silica gel column chromatography using ethyl acetate/hexane (10:90 v/v) as eluent to afford tert-butyl 5-bromo-3-(N-(tert-butoxycarbonyl)-N-methylalanyl)-1H-indole-1-carboxylate (10) as yellow sticky gum (3 g, 54%). ¹H NMR (400 MHz, DMSO-d₆) δ 8.59 & 8.41 (s, 1H), 8.33 (d, J=1.92 Hz, 1H), 8.04-8.00 (t, J=8.08 Hz, 7.76 Hz, 1H), 7.58-7.55 (dd, J=1.56 Hz, 8.92 Hz, 1H), 5.41 & 5.06 (s, 1H), 2.81 & 2.63 (s, 3H), 1.62 (s, 9H), 1.37-1.23 (m, 12H). MS (ES) C₂₂H₂₉BrN₂O₅ requires 480, found 383 [M−100+2], rotamers observed.

### Step 5: Synthesis of 1-(5-bromo-1H-indol-3-yl)-2-(methylamino) propan-1-one hydrochloride(BK-5Br-NM-AMT)

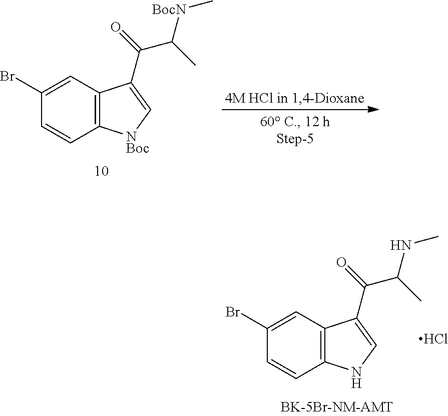

To a stirred solution of tert-butyl 5-bromo-3-(N-(tert-butoxycarbonyl)-N-methylalanyl)-1H-indole-1-carboxylate (10) (3 g, 6.23 mmol, 1 eq.) in dry DCM (20 ml) was added 4M HCl in 1,4 dioxane (30 ml) at 0° C. and the resulting reaction mixture was allowed to stir at 60° C. for 12 hrs. Upon completion of reaction (monitored by TLC, 10% EA in Hexane), the solvent were evaporated and the crude was washed twice with diethyl ether (2×50 ml) and pentane (1×50 ml) and dried under vacuum to afford 1-(5-bromo-1H-indol-3-yl)-2-(methylamino) propan-1-one hydrochloride (BK-5Br-NM-AMT) as off white solid (1.8 g, 81%). ¹H NMR (400 MHz, DMSO-d₆) δ 12.69 (s, 1H), 9.39-9.07 (bs, 2H), 8.61 (s, 1H), 8.30 (d, J=1.4 Hz, 1H), 7.53 (d, J=8.6 Hz, 1H), 7.43-7.40 (dd, J=1.68 Hz, 8.6 Hz, 1 Hz), 4.86-4.81 (q, 1H), 2.55 (s, 3H), 1.51 (d, J=6.92 Hz, 3H). MS (ES) C₁₂H₁₃BrN₂O requires 280, found 281 [M+H]⁺. HPLC: Purity (λ 220 nm): 98.46%.

### Additional Synthetic Examples

Starting with known starting materials the skilled artisan can synthesize compounds of the present invention with conventional methods and the teachings of this patent application. For example, pyrrolidine containing compounds of the present invention can be prepared from commercially available protected starting materials.

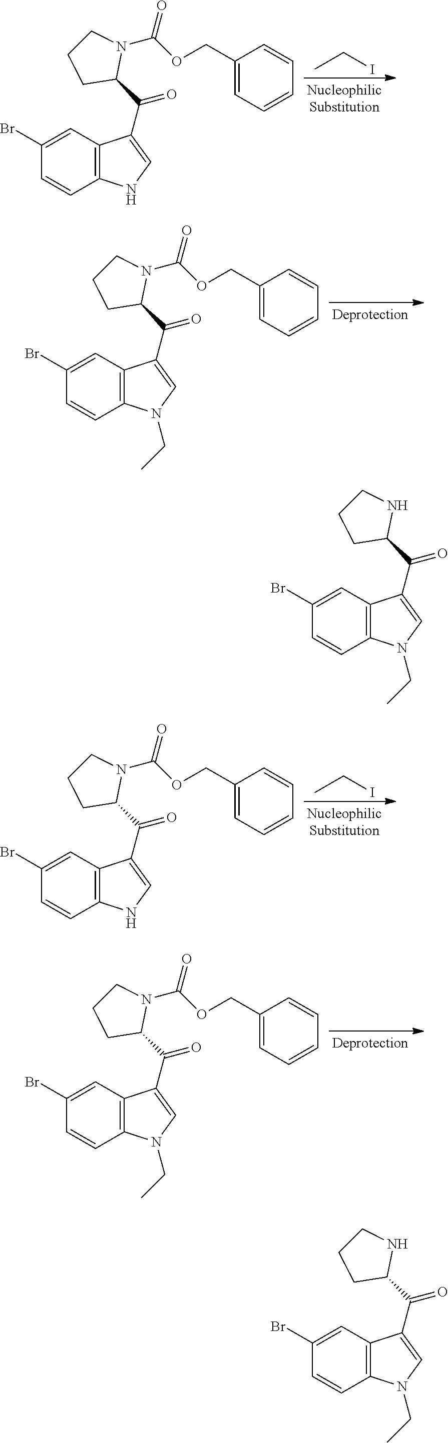

Additional compounds of the present invention can by synthesized by changing the order of reactions and if necessary switching protecting groups.

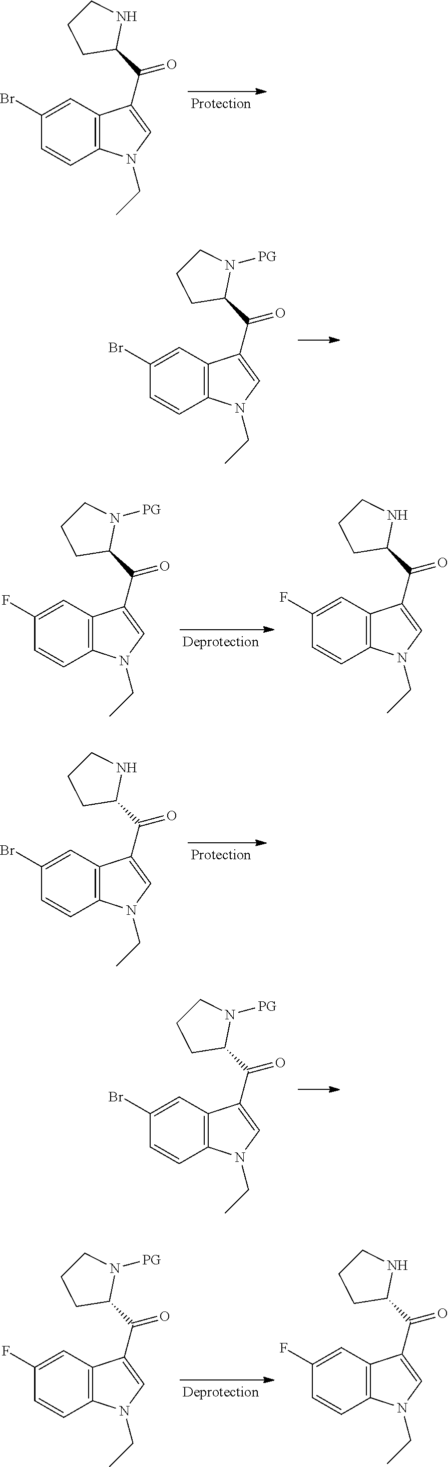

These techniques can be further modified by using different indoles to provide additional compounds of the present invention.

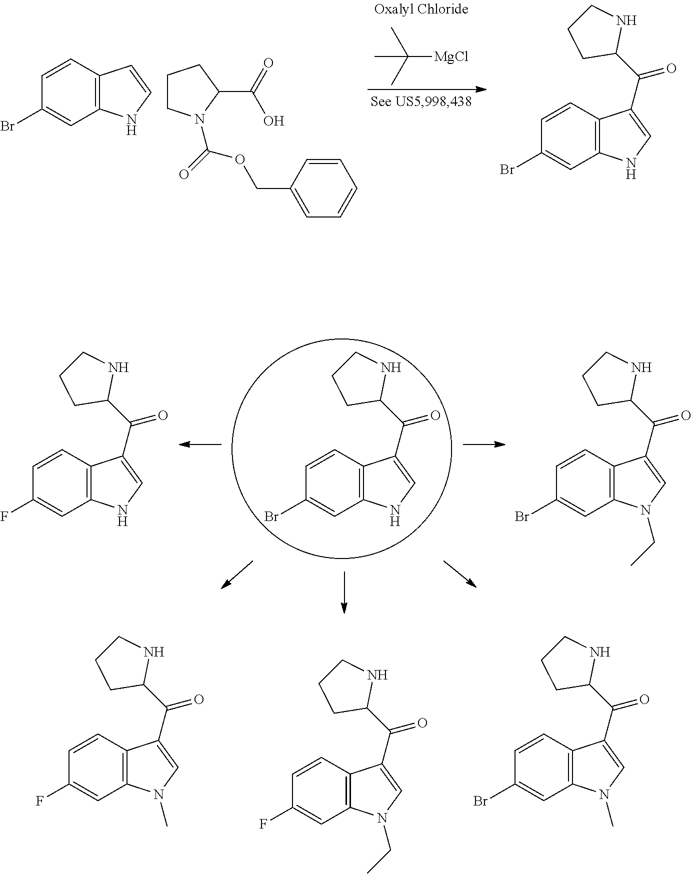

### Preparation of Enantiomeric Compounds

Various methods are known in the art for preparing optically active forms and determining activity. Such methods include standard processes described herein and other similar assays which are well known in the art. Examples of methods that can be used to obtain optical isomers of the compounds according to the present disclosure include but are not limited to the following:
a) physical separation of crystals whereby macroscopic crystals of the individual enantiomers are manually separated. This technique may particularly be used if crystals of the separate enantiomers exist (i.e., the material is a conglomerate), and the crystals are visually distinct;
b) simultaneous crystallization whereby the individual enantiomers are separately crystallized from a solution of the racemate, possible only if the latter is a conglomerate in the solid state;
c) enzymatic resolutions whereby partial or complete separation of a racemate by virtue of differing rates of reaction for the enantiomers with an enzyme;
d) enzymatic asymmetric synthesis, a synthetic technique whereby at least one step of the synthesis uses an enzymatic reaction to obtain an enantiomerically pure or enriched synthetic precursor of the desired enantiomer;
e) chemical asymmetric synthesis whereby the desired enantiomer is synthesized from an achiral precursor under conditions that produce asymmetry (i.e., chirality) in the product, which may be achieved using chiral catalysts or chiral auxiliaries;
f) diastereomer separations whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
g) first- and second-order asymmetric transformations whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomers;
h) kinetic resolutions comprising partial or complete resolution of a racemate (or of a further resolution of a partially resolved compound) by virtue of unequal reaction rates of the enantiomers with a chiral, enantiomerically enriched reagent or catalyst under kinetic conditions;
i) enantiospecific synthesis from enantiomerically enriched precursors whereby the desired enantiomer is obtained from non-chiral starting materials and where the stereochemical integrity is not or is only minimally compromised over the course of the synthesis;
j) chiral liquid chromatography whereby the enantiomers of a racemate are separated in a liquid mobile phase by virtue of their differing interactions with a stationary phase. The stationary phase can be made of chiral material or the mobile phase can contain an additional chiral material to provoke the differing interactions;
k) chiral gas chromatography whereby the racemate is volatilized and enantiomers are separated by virtue of their differing interactions in the gaseous mobile phase with a column containing a fixed enantiomerically enriched chiral adsorbent phase;
l) extraction with chiral solvents whereby the enantiomers are separated by virtue of preferential dissolution of one enantiomer into a particular chiral solvent; and
m) transport across chiral membranes whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the enantiomerically enriched chiral nature of the membrane, which allows only one enantiomer of the racemate to pass through.

Where diastereomers exist, the compounds can be used in any diastereomeric form or mixture of forms that provides the appropriate therapeutic effect for the patient, as taught herein. Therefore, in one embodiment, the compounds of the present invention can be administered in a racemic mixture, as the R-enantiomer, as the S-enantiomer, or as an enantiomerically enriched mixture, or a diastereomeric form.

The following compounds indicate where primary stereocenters exist when the designated R group is not hydrogen. In certain embodiments, the enantiomers of the present invention include:

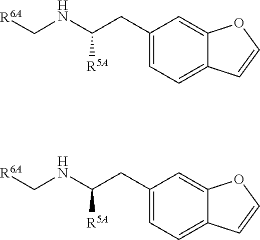

wherein R^{5A} is not hydrogen.

In certain embodiments, the enantiomers of the present invention include:

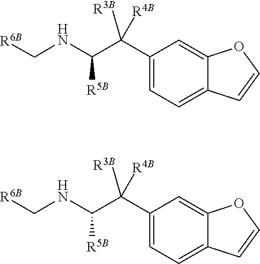

wherein R^{5B} is not hydrogen.

In certain embodiments, the enantiomers of the present invention include:

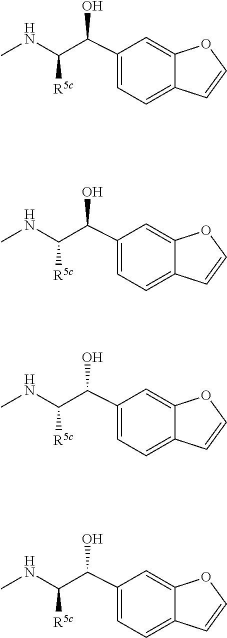

wherein R^{5C} is not hydrogen.

In certain embodiments, the enantiomers of the present invention include:

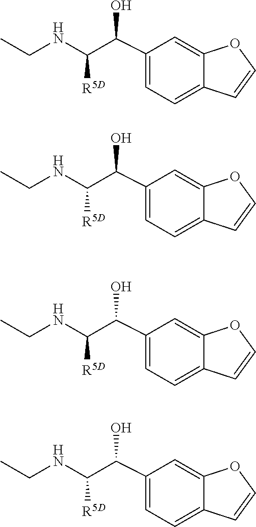

wherein R^{5D} is not hydrogen.

In certain embodiments, the enantiomers of the present invention include:

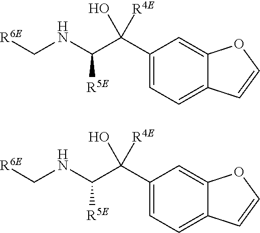

wherein R^{5E} is not hydrogen.

In certain embodiments, the enantiomers of the present invention include:

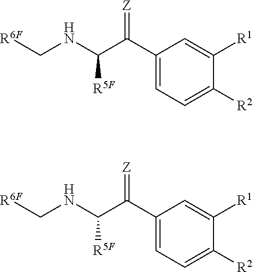

wherein R^{5F} is not hydrogen.

In certain embodiments, the enantiomers of the present invention include:

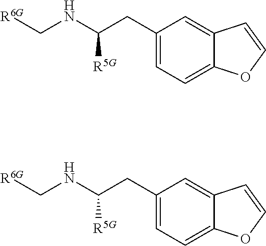

wherein R^{5G} is not hydrogen.

In certain embodiments, the enantiomers of the present invention include:

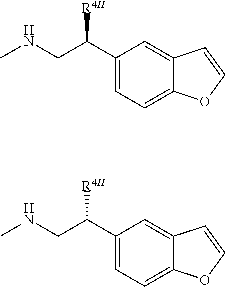

wherein R^{4H} is not hydrogen.

In certain embodiments, the enantiomers of the present invention include:

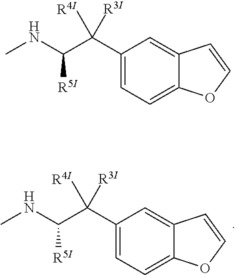

In certain embodiments, the enantiomers of the present invention include:

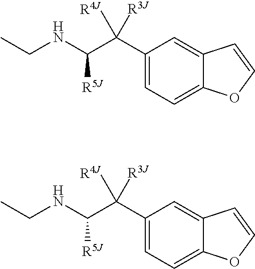

wherein R^{5J} is not hydrogen.

In certain embodiments, the enantiomers of the present invention include:

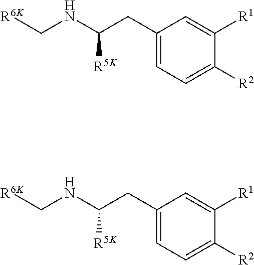

wherein R^{5K} is not hydrogen.

In certain embodiments, the enantiomers of the present invention include:

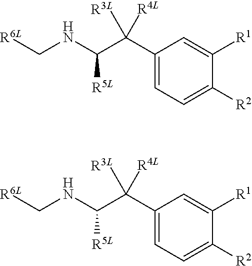

wherein R^{5L} is not hydrogen.

In certain embodiments, the enantiomers of the present invention include:

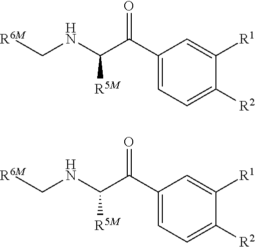

wherein R^{5M} is not hydrogen.

### Non-Limiting Examples of Chiral Synthetic Methods Include

### Preparation of (R)-1-(benzofuran-6-yl)-N-methylbutan-2-amine hydrochloride (R-6-MBPB)

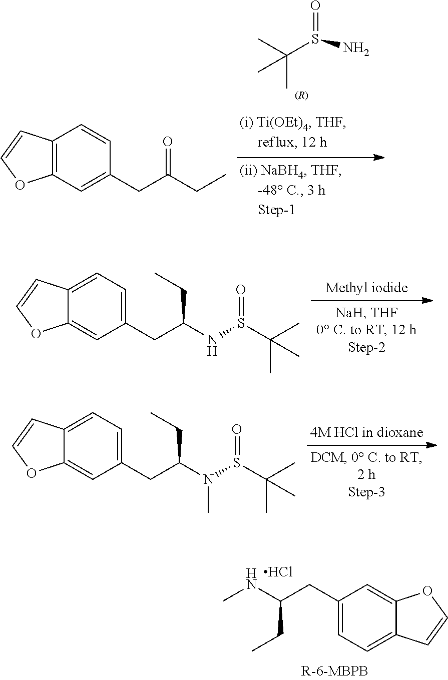

### Step-1

To a stirred solution of 1-(benzofuran-6-yl)butan-2-one (500 mg, 2.656 mmol, 1.0 equiv.) in THF (20 mL) was added Ti(OEt)₄ (2.0 mL, 9.297 mmol, 3.5 equiv.) and (R)-2-methylpropane-2-sulfinamide (dissolved in 5 mL THF) (321 mg, 2.656 mmol, 1.0 equiv.). Then the reaction mixture was allowed to stir at 70° C. for 12 h. After completion of the reaction (monitored by TLC, 50% EA in Hexane). The reaction mixture was cooled to 0° C. and NaBH₄ (400 mg, 10.625 mmol, 4.0 equiv.) was added into it at −48° C. and the reaction mixture was allowed to stir at −45° C. for 3 h. TLC (50% EA-Hexane) showed the formation of a new polar spot. Crude LCMS analysis showed formation of the desired product. The reaction mixture was taken to RT and then it was quenched with Methanol and Sat. NaCl solution (White Precipitate observed). The reaction mixture was filtered through celite bed, then washed with Methanol and DCM. Collect the organic layer and evaporated under vacuo to remove the volatiles. Then the reaction mixture was diluted with ethyl acetate, washed with water, and brine. Collect the organic layer, dried over anhydrous sodium sulphate and concentrated under vacuo to get the crude compound ((R)-N-(R)-1-(benzofuran-6-yl)butan-2-yl)-2-methylpropane-2-sulfinamide (900 mg). The crude compound was used in the next step without further purification. LCMS: Rt 1.98 min. MS (ES) C₁₆H₂₃NO₂S requires 293, found 294 [M+H]⁺. Mass of the other isomer was observed in LCMS.

### Step-2

To a stirred solution of ((R)-N-(R)-1-(benzofuran-6-yl)butan-2-yl)-2-methylpropane-2-sulfinamide (900 mg, 3.06 mmol, 1 equiv.) (Sealed tube) in THE was added NaH (420 mg, 10.22 mmol, 3.0 equiv.) at 0° C. After that methyl iodide (0.85 mL, 13.63 mmol, 4 equiv.) was added to this reaction mixture. The reaction mixture was stirred at RT for 12 h. After completion of the reaction, the reaction mixture was quenched with ice-cold water and extracted with DCM. The organic part was concentrated, and the crude material was purified by silica gel column chromatography using ethyl acetate/hexane (10:90 v/v) as eluent to afford (R)-N-(R)-1-(benzofuran-6-yl)butan-2-yl)-N,2-dimethylpropane-2-sulfinamide as sticky gum (280 mg, 27%). ¹H NMR (400 MHz, DMSO) δ 7.92 (d, J=2 Hz, 1H), 7.56 (d, J=7.88 Hz, 1H), 7.41 (s, 1H), 7.10 (d, J=7.76 Hz, 1H), 6.90 (s, 1H), 2.94-2.82 (m, 2H), 2.45 (s, 3H), 1.57-1.54 (m, 1H), 1.40-1.35 (m, 1H), 1.23 (m, 1H), 1.08 (s, 9H), 0.86 (t, J=7.28 Hz, 3H). LCMS: Rt 1.88 min. MS (ES) C₁₇H₂₅NO₂S requires 307, found 308 [M+H]⁺.

### Step-3

To a stirred solution of (R)-N-(R)-1-(benzofuran-6-yl)butan-2-yl)-N,2-dimethylpropane-2-sulfinamide (3) (100 mg, 0.325 mmol, 1.0 equiv.) in DCM was added 4M HCl in dioxane (0.5 mL) at 0° C. The reaction mixture was stirred at RT for 2 h. After completion of the reaction, evaporated the solvent in vacuo and solid was formed which was washed with ether to afford (R)-1-(benzofuran-6-yl)-N-methylbutan-2-amine hydrochloride as a white solid (52 mg, 78.65%). ¹H NMR (400 MHz, DMSO) δ 8.96-8.88 (bs, 1H), 8.81-8.75 (bs, 1H), 7.97 (d, J=2.04 Hz, 1H), 7.62 (d, J=7.92 Hz, 1H), 7.57 (s, 1H), 7.19 (d, J=7.96 Hz, 1H), 6.94 (d, J=1.44 Hz, 1H), 3.38-3.35 (m, 1H), 3.20-3.15 (dd, J=5.12 Hz, 5.20 Hz, 1H), 2.93-2.88 (m, 1H), 2.55 (s, 3H), 1.61-1.49 (m, 2H), 0.93 (t, J=7.44 Hz, 3H). LCMS: Rt 1.98 min. MS (ES) C₁₃H₁₈ClNO requires 203, found 204 [M+H]⁺. HPLC: Rt 4.28 min, Purity (λ 250 nm): 98.09%, chiral purity: Rt 6.24 min, 99.17%, ee: 98.34.

### Preparation of (S)-1-(benzofuran-6-yl)-N-methylbutan-2-amine hydrochloride (S-6-MBPB)

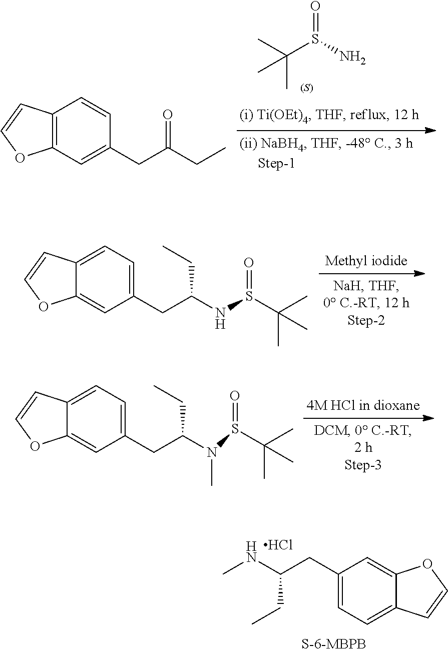

### Step-1

To a stirred solution of 1-(benzofuran-6-yl) butan-2-one (500 mg, 2.656 mmol, 1.0 equiv.) in dry THF (20 mL) was added Ti(OEt)₄ (2.0 mL, 9.297 mmol, 3.5 equiv.) and (S)-2-methylpropane-2-sulfinamide(dissolved in 5 mL THF) (321 mg, 2.656 mmol, 1.0 equiv.) at RT. The resulting reaction mixture was continued to stir at 70° C. for 12 h. TLC (50% EtOAc-Hex) monitoring showed the formation of polar spot. The reaction mixture was cooled to 0° C. and NaBH₄ (400 mg, 10.625 mmol, 4.0 equiv.) was added into the reaction mixture at −48° C. and the reaction mixture was allowed to stir at −45° C. for 3 h. After completion, (monitored by TLC, 50% EA-Hexane), the reaction mixture was taken to RT, quenched with Methanol and Saturated NaCl solution (White Precipitate observed). The reaction mixture was filtered through celite bed, washed the celite bed with Methanol and DCM, and evaporated under vacuo to remove the volatiles. Then the reaction mixture was taken in ethyl acetate, washed with water, followed by brine then dried over sodium sulphate, and concentrated under vacuo to get the crude compound (S)-N-((S)-1-(benzofuran-6-yl) butan-2-yl)-2-methylpropane-2-sulfinamide (900 mg) as a colorless sticky liquid, which was forwarded to the next step without purification. LCMS: Rt 3.53 min. MS (ES) C₁₆H₂₃NO₂S requires 293, found 294 [M+H]⁺. Mass of other isomer observed in LCMS.

### Step-2

To a stirred solution of (S)-N-((S)-1-(benzofuran-6-yl) butan-2-yl)-2-methylpropane-2-sulfinamide (crude) (900 mg, 3.067 mmol, 1.0 equiv.) (Sealed tube) in dry THE (20.0 mL) was added (60%) NaH (370 mg, 9.202 mmol, 3.0 equiv.) at 0° C. After that methyl iodide (0.9 mL, 12.269 mmol, 4 equiv.) was added to the reaction mixture. The resulting reaction mixture was stirred at RT for 12 h. After completion (Monitored by TLC, 40% EA in Hex), the reaction mixture was quenched with cold water (30 mL) and extracted with ethyl acetate (200 mL) and then washed with NaCl solution. The collected organic layer was dried over sodium sulphate and concentrated under reduced pressure. The crude compound was purified by combi flash column chromatography eluted with 10%-15% ethyl acetate in hexane to afford (S)-N-((S)-1-(benzofuran-6-yl)butan-2-yl)-N,2-dimethylpropane-2-sulfinamide (280.0 mg) as a colorless sticky liquid. ¹H NMR (400 MHz, CDCl₃) δ 7.56 (m, 1H), 7.50 (d, J=7.88 Hz, 1H), 7.31 (bs, 1H), 7.07 (d, J=7.84 Hz, 1H), 6.71 (bs, 1H), 3.31 (bs, 1H), 3.17-3.12 (m, 1H), 2.84-2.78 (m, 1H), 2.55 (s, 3H), 1.52-1.50 (m, 2H), 1.19 (s, 9H), 0.89-0.85 (m, 3H). LCMS: Rt 3.64 min. MS (ES) C₁₇H₂₅NO₂S requires 307, found 307.7 [M+H]⁺.

### Step-3

To a stirred solution of (S)-N-((S)-1-(benzofuran-6-yl) butan-2-yl)-N,2-dimethylpropane-2-sulfinamide (280 mg, 0.867 mmol, 1.0 equiv.) in DCM (5.0 mL) was added 4M HCl in dioxane (2.0 mL) at 0° C. The resulting reaction mixture was stirred at 0° C.-RT for 2 h. After completion (Monitoring by TLC, 20% EA in Hex), the excess solvent was evaporated under reduced pressure to get the crude, which was washed with diethyl ether and dried to afford S-6-MBPB (150.0 mg) as a white solid. ¹H NMR (400 MHz, DMSO-d₆) δ 9.05-8.90 (bm, 2H), 7.96 (d, J=1.96 Hz, 1H), 7.62-7.60 (d, J=7.96 Hz, 1H), 7.57 (s, 1H), 7.19 (d, J=7.84 Hz, 1H), 6.93 (d, J=1.16 Hz, 1H), 3.22-3.18 (dd, J=13.76 Hz, 4.96 Hz, 1H), 2.94-2.88 (m, 1H), 2.54 (s, 3H), 1.62-1.48 (m, 2H), 0.92 (t, J=7.44, 7.48 Hz, 3H). LCMS: Rt 1.98 min. MS (ES) C₁₃H₁₈ClNO requires 203, found 204 [M+H]⁺. HPLC: Rt 6.97 min. Purity (λ 210 nm): 98.11% Chiral purity: Rt 7.38: 97.56%, ee is 95.12.

### Preparation of (S)-1-(benzofuran-5-yl)-N-methylbutan-2-amine hydrochloride (S-5-MBPB)

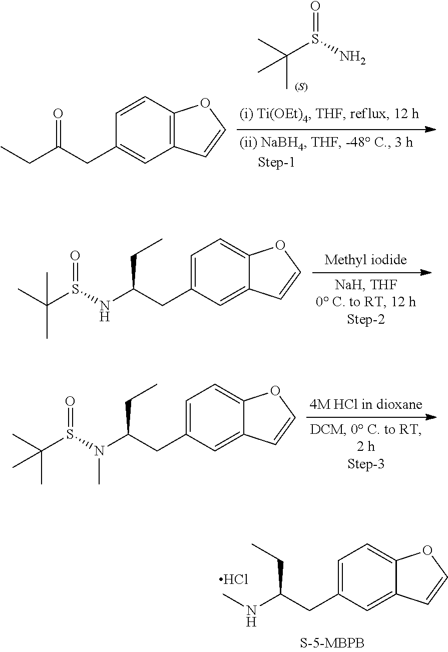

### Step-1

To a stirred solution of 1-(benzofuran-5-yl) butan-2-one (2 g, 10.625 mmol, 1 equiv.) in dry THF (10 mL) was added Ti(OEt)₄ (7.797 mL, 37.189 mmol, 3.5 equiv.) and (S)-2-methylpropane-2-sulfinamide (1.288 g, 10.625 mmol, 1 equiv.) (dissolved in 10 mL dry THF) and the resulting reaction mixture was allowed to stir at 70° C. for 12 h. Upon completion, monitored by TLC (50% EA in Hexane), the reaction mixture was cooled to 0° C., gradually to −48° C. and NaBH₄ (1.615 g, 42.501 mmol, 4 equiv.) was added into the reaction mixture at −48° C. and the resulting reaction mixture was allowed to stir at −48° C. for 3 h. Upon completion, monitored by TLC (50% EA in Hexane), the reaction mixture was slowly warm to room temperature and was quenched with Methanol and saturated NaCl solution (until a white precipitate was observed). The reaction mixture was then filtered through celite bed, washed the celite bed with methanol (2×100 mL) and ethyl acetate (2×100 mL), then evaporated under vacuum to remove the volatiles. Then the reaction mixture was diluted with ethyl acetate, washed with water, followed by brine solution. The combined organic layer was collected and dried over anhydrous sodium sulphate, the solvent was removed under vacuum to afford the crude (S)-N-((S)-1-(benzofuran-5-yl) butan-2-yl)-2-methylpropane-2-sulfinamide as yellow sticky gum (2.8 g, 89.81%). Proceed for the next step without further purification. LCMS: Rt 1.94 min. MS (ES) C16H23NO2S, requires 293, found 294 [M+H]⁺.

### Step-2

To a stirred solution of crude (S)-N-((S)-1-(benzofuran-5-yl) butan-2-yl)-2-methylpropane-2-sulfinamide (3.94 g, 13.441 mmol, 1 equiv.) in dry THE (40 mL) (In a sealed tube) was added NaH (60% in mineral oil) (0.968 g, 40.322 mmol, 3 equiv.) at 0° C. and the resulting reaction mixture was allowed to stir at 0° C. for 30 min. Then methyl iodide (3.347 mL, 53.763 mmol, 4 equiv.) was added at 0° C. and the resulting reaction mixture was allowed to stir at room temperature for 12 h. Upon completion, monitored by TLC (50% EA in Hexane), the reaction mixture was quenched with ice water, extracted with ethyl acetate (2×200 mL), then washed with saturated ammonium chloride solution followed by brine solution. The combined organic layer was dried over anhydrous sodium sulphate, filtered and the solvent was removed under vacuum to get the crude which was purified by silica gel column chromatography using ethyl acetate/hexane (50:50 v/v) as eluent to afford (S)-N-((S)-1-(benzofuran-5-yl)butan-2-yl)-N,2-dimethylpropane-2-sulfinamide as light yellow gum (3.8 g, 91.96%). We can separate other minor isomer formed by column chromatography at this step. ¹H NMR (400 MHz, CDCl₃) δ 7.58 (d, J=2.04 Hz, 1H), 7.41-7.39 (bs, 2H), 7.11-7.09 (dd, J=1.36 Hz, J=8.40 Hz, 1H), 6.70 (d, J=1.28 Hz, 1H), 3.31-3.28 (m, 1H), 3.14-3.09 (dd, J=4.16 Hz, 13.44 Hz, 1H), 2.55 (s, 3H), 1.55-1.48 (m, 2H), 1.19 (s, 9H), 0.89 (t, J=7.32 Hz, 7.36 Hz, 3H). LCMS: Rt2.08 min. MS (ES) C17H25NO2S, requires 307, found 308 [M+H]⁺.

### Step-3

To a stirred solution of (S)-N-((S)-1-(benzofuran-5-yl) butan-2-yl)-N,2-dimethylpropane-2-sulfinamide (736 mg, 2.396 mmol, 1 equiv.) in dry DCM (10 mL) was added 4(M) HCl in 1,4 dioxane (5 mL) at 0° C. and the resulting reaction mixture was allowed to stir at room temperature for 2 h. After completion of the reaction, the solvent was evaporated and the crude was washed twice with diethyl ether (2×100 mL) and pentane and then dried under vacuum to afford S-5-MBPB (410 mg, 84.17%) as a white solid. ¹HNMR(400 MHz, DMSO-d₆) δ 8.94-8.79 (bs, 2H), 7.99 (d, J=1.96 Hz, 1H), 7.57-7.55 (m, 2H), 7.24 (d, J=8.36 Hz, 1H), 6.93 (d, J=1.24 Hz, 1H), 3.19-3.14 (dd, J=4.96 Hz, J=13.76 Hz, 1H), 2.91-2.86 (q, 1H), 2.55 (s, 3H), 1.60-1.51 (m, 2H), 0.92 (t, J=7.44 Hz, 7.48 Hz, 3H) LCMS: Rt 1.39 min. MS (ES) C13H17NO, requires 203.13, found 204 [M+H]⁺. HPLC: Rt 4.75 min. Purity (λ 260 nm): 96.54%, chiral purity: Rt 3.18 min, 99.05%, ee: 98.11.

### Preparation of (R)-1-(benzofuran-5-yl)-N-methylbutan-2-amine (R-5-MBPB)

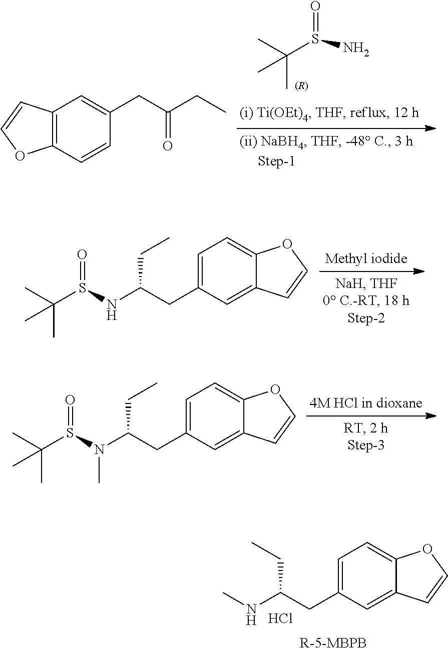

### Step-1

To a stirred solution of 1-(benzofuran-5-yl) butan-2-one (2.0 g, 10.625 mmol, 1.0 equiv.) in THF (40.0 mL) was added Ti(OEt)₄ (7.7 mL, 37.19 mmol, 3.5 equiv.) and (R)-2-methylpropane-2-sulphinamide (dissolved in 5 ml THF) (1.3 g, 10.6 mmol, 1.0 equiv.) then the reaction mixture was allowed to stir at 70° C. for 12 h. TLC (50% EA in Hexane) monitoring showed the formation of new polar spot. The reaction mixture was cooled to 0° C. and NaBH₄ (1.7 g, 42.5 mmol, 4.0 equiv.) was added into the reaction mixture at −45° C. and the reaction mixture was allowed to stir at −45° C. for 3 hrs. TLC (50% EA-Hexane) showed formation of new polar spot. Crude LCMS showed the formation of the desired product. The reaction mixture was taken to RT and it was quenched with Methanol and Sat. NaCl solution (White Precipitate observed). The reaction mixture was filtered through a celite bed, washed the celite bed with Methanol and DCM, and evaporated under vacuo to remove the volatiles. Then it was diluted with ethyl acetate, washed with water, followed by brine then dried over sodium sulphate, and concentrated under vacuo to get the crude (R)-N-((R)-1-(benzofuran-5-yl)butan-2-yl)-2-methylpropane-2-sulfinamide (2 g, 64%), which was used in next step without further purification. LCMS: Rt 1.94 min. MS (ES) C16H23NO2S requires 293, found 294 [M+H]⁺.

### Step-2

To a stirred solution of (R)-N-((R)-1-(benzofuran-5-yl)butan-2-yl)-2-methylpropane-2-sulfinamide (2.0 gm, 6.816 mmol, 1.0 equiv.) (Sealed tube) in THF (20 mL) was added NaH (60%) (818 mg, 20.448 mmol, 3.0 equiv.) Portion-wise at 0° C. and stirred at same temperature for 30 min. Then methyl iodide (2 mL, 27.264 mmol, 4.0 equiv.) was added and the resulting reaction mixture was stirred at RT for 16 h. After completion (monitoring by TLC), the reaction mixture was quenched with cold water (50 mL) and Ethyl acetate (100 mL). The organic part was collected and washed with sat. NaHCO₃(20 mL) solution followed by brine. The organic layer was collected and dried over anhydrous sodium sulfate, filtered and evaporated under reduced pressure to get the crude which was purified by silica gel (100-200 mesh) column chromatography and elute with 15% ethyl acetate-hexane to get the desired product (900 mg, 43%) as a colorless sticky gum. ¹H NMR (400 MHz, DMSO-d₆) δ 7.94-7.92 (m, 1H), 7.50-7.45 (m, 2H), 7.13 (d, J=8.36, 1H), 6.89 (s, 1H), 3.27-3.25 (m, 1H), 2.92-2.79 (m, 2H), 2.45 (m, 3H), 1.58-1.50 (m, 1H), 1.42-1.35 (m, 1H), 1.08 (bs, 9H), 0.83 (t, J=7.32 Hz, 3H). LCMS: Rt 2.03 min. MS (ES) C₁₇H₂₅NO₂S requires 307, found 308 [M+H]⁺.

### Step-3

To a stirred solution of (R)-N-((R)-1-(benzofuran-5-yl) butan-2-yl)-N,2-dimethylpropane-2-sulfinamide (900 mg, 2.927 mmol, 1 equiv.) in dry DCM (10 mL) was added 4M HCl in 1,4 dioxane (4.0 mL) at 0° C. and then the resulting reaction mixture was allowed to stir at room temperature for 2 h. Upon completion of the reaction, the solvent was evaporated, and the crude was washed twice with diethyl ether (2×20 ml) and pentane and dried under vacuum to afford R-5-MBPB (550 mg, 78%) as off white solid. ¹HNMR (400 MHz, DMSO-d₆) δ 8.85-8.72 (bm, 2H), 7.99 (d, J=2 Hz, 1H), 7.57-7.55 (m, 2H), 7.24 (d, J=9.52 Hz, 1H), 6.93 (d, J=1.8 Hz, 1H), 3.17-3.13 (dd, J=4.8 Hz, 5.2 Hz 1H), 2.91-2.85 (q, 1H), 2.56 (bs, 3H), 1.61-1.49 (m, 2H), 1.04 (s, 1H), 0.89 (t, J=7.44 Hz, 7.52 Hz, 3H). LCMS: Rt 1.94 min. MS (ES) C13H17NO requires 203, found 204 [M+H]⁺. HPLC: Rt 6.51 min. Purity (λ 220 nm): 95.08%, chiral purity: Rt 3.82 min. 99.51%, ee 99.03.

### Preparation of (S)-1-(benzofuran-5-yl)-N-methylpropan-2-amine hydrochloride (S-5-MAPB)

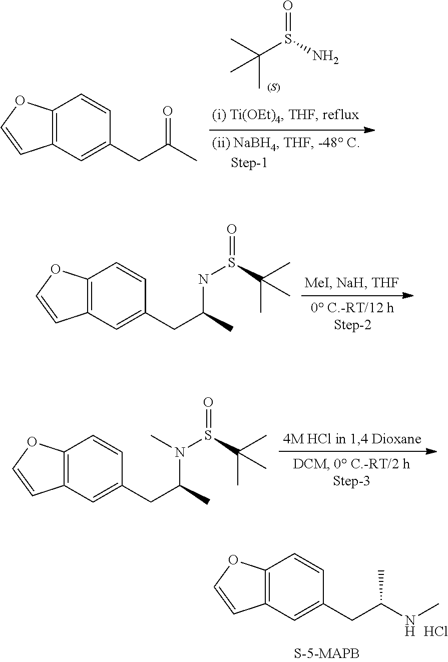

Actual stereochemistry mentioned here based on literature report J. Med. Chem. 2017, 60, 3958-3978.

### Step-1

To a stirred solution of 1-(benzofuran-5-yl)propan-2-one (5 g, 28.70 mmol, 1 eq.) in dry THF (100 ml) was added Ti(OEt)₄ (21.06 ml, 100.45 mmol, 3.5 eq.) and (S)-2-methylpropane-2-sulfinamide (3.47 g, 28.73 mmol, 1 eq.) (dissolved in 20 ml dry THF) and the resulting reaction mixture was allowed to stir at 70° C. for 12 hrs. Upon completion (monitored by TLC, 50% EA in Hexane), the reaction mixture was cooled to 0° C., gradually to −48° C. and NaBH₄ (4.34 g, 114.81 mmol, 4 eq.) (dissolved in 20 ml dry THF) was added into the reaction mixture at −48° C. and the resulting reaction mixture was allowed to stir at −48° C. for 3 hrs. Upon completion (monitored by TLC, 50% EA in Hexane), the reaction mixture was taken to room temperature and was quenched with Methanol and Sat. NaCl solution (until white precipitate observed). The reaction mixture was then filtered through celite bed, washed with methanol (2×100 ml) and ethyl acetate (2×100 ml), evaporated under vacuum to remove the volatiles. Then the reaction mixture was extracted with ethyl acetate, washed with water, followed by brine solution. Combined organic layer was dried over anhydrous sodium sulphate, solvent was removed under vacuum to afford crude (S)-N-((S)-1-(benzofuran-5-yl)propan-2-yl)-2-methylpropane-2-sulfinamide as yellow sticky gum (6.5 g, 81%). Crude ¹H NMR (400 MHz, DMSO-d₆) δ 7.94 (d, J=7.8 Hz, 1H), 7.50 (m, 2H), 7.14 (m, 1H), 6.90 (d, J=6.36 Hz, 1H), 6.90 (d, J=6.36 Hz, 1H), 4.97 (d, J=5.96 Hz, 1H), 3.48 (m, 1H), 3.08 (m, 1H), 2.76 (m, 1H), 1.18 (m, 12H). LCMS: Rt 1.83 min. MS (ES) C15H21NO2S requires 279, found 280 [M+H]+.

### Step-2

To a stirred solution of (S)-N-((S)-1-(benzofuran-5-yl)propan-2-yl)-2-methylpropane-2-sulfinamide (7 g, 25 mmol, 1 eq.) in dry THE (50 mL) (In a sealed tube) was added NaH (60%) (2 g, 50 mmol, 2 eq.) at 0° C. and the resulting reaction mixture was allowed to stir at 0° C. for 30 min. Then Iodomethane (3.11 ml, 50 mmol, 2 eq.) was added at 0° C. and the resulting reaction mixture was allowed to stir at room temperature for 12 h. Upon completion (monitored by TLC, 50% EA in Hexane), the reaction mixture was quenched with ice water, extracted with ethyl acetate (2×200 ml), washed with saturated ammonium chloride solution, followed by brine solution. Combined organic layer was dried over anhydrous sodium sulphate, solvent was removed under vacuum and purified by silica gel column chromatography using ethyl acetate/hexane (50:50 v/v) as eluent to afford (S)-N-((S)-1-(benzofuran-5-yl)propan-2-yl)-N,2-dimethylpropane-2-sulfinamide as light-yellow gum (4 g, 54%). ¹H NMR (400 MHz, DMSO-d₆) δ 7.94 (s, 1H), 7.49 (t, J=8.4 Hz, 9.04 Hz, 2H), 7.14 (d, J=8.2, 1H), 6.89 (s, 1H), 3.55 (m, 1H), 2.92 (m, 1H), 2.88 (m, 1H), 2.51 (s, 3H), 1.27 (m, 3H), 1.07 (S, 9H). LCMS: Rt 1.91 min. MS (ES) C16H23NO2S requires 293, found 294 [M+H]+.

### Step-3

To a stirred solution of (S)-N-((S)-1-(benzofuran-5-yl)propan-2-yl)-N,2-dimethylpropane-2-sulfinamide (3) (7 g, 23.89 mmol, 1 eq.) in dry DCM (35 mL) was added 4M-HCl in 1,4 dioxane (70 mL) at 0° C. and the resulting reaction mixture was allowed to stir at room temperature for 2 h. Upon completion of reaction (monitored by TLC, 30% EA in Hexane), the solvent was evaporated, and the crude was washed twice with diethyl ether (2×60 ml) and pentane and dried under vacuum to afford (S)-1-(benzofuran-5-yl)-N-methylpropan-2-amine hydrochloride (Compound-9S) (5 g, 97%) as off white solid. ¹HNMR (400 MHz, DMSO-d₆) δ 9.06 (bs, 2H), 7.99 (d, J=1.88 Hz, 1H), 7.57 (m, 2H), 7.21 (d, J=8.28 Hz, 1H), 6.93 (d, J=1.32 Hz, 1H), 3.33 (m, 1H), 3.26 (m, 1H), 2.77 (q, 1H), 2.56 (s, 3H), 1.11 (d, J=6.4 Hz, 3H), LCMS: Rt 1.33 min. MS (ES) C12H15NO requires 189, found 190 [M+H]⁺. HPLC: Rt 5.73 min. Purity (λ 250 nm): 99.81%.

### Preparation of (R)-1-(benzofuran-5-yl)-N-methylpropan-2-amine hydrochloride (R-5-MAPB)

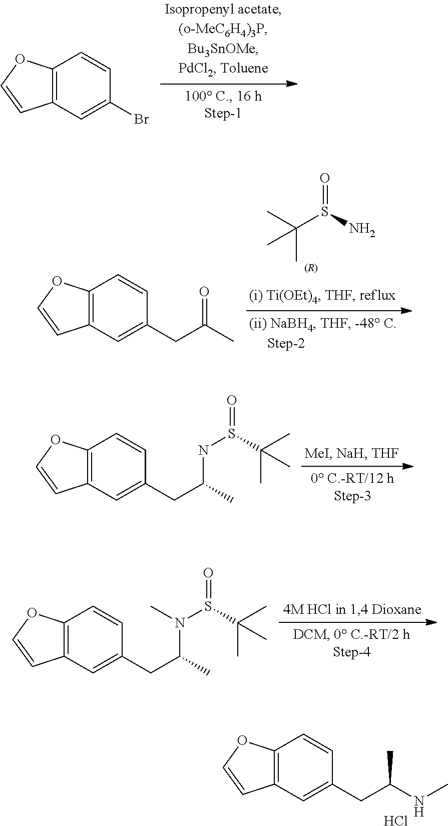

Actual stereochemistry mentioned here based on literature report J. Med. Chem. 2017, 60, 3958-3978.

### Step-1

To a stirred solution of 5-bromobenzofuran (20 g, 101.52 mmol, 1 eq.) in dry Toluene (400 ml) was added tri(o-tolyl)phosphine (1.84 g, 6.091 mmol, 0.06 eq.), tributyl tin methoxide (48.89 mL, 152.28 mmol, 1.5 eq.) and Isopropenyl acetate (16.99 mL, 156.34 mmol, 1.54 eq.) and the resulting reaction mixture was degassed under nitrogen for 15 minutes. Then palladium (II) chloride (1.26 g, 7.10 mmol, 0.07 eq.) was added to the reaction mixture and the resulting reaction mixture was heated to 100° C. for 16 hrs. Upon completion, monitored by TLC (10% EA in Hexane), the reaction mixture was filtered through celite bed, extracted with ethyl acetate (2×400 ml), washed with water, followed by saturated potassium fluoride solution, and brine solution. Combined organic layer was dried over anhydrous sodium sulphate, solvent was removed under vacuum and purified by silica gel column chromatography using ethyl acetate/hexane (10:90 v/v) as eluent to afford 1-(benzofuran-5-yl)propan-2-one as light yellow gum (17 g, 96%).

¹H NMR (400 MHz, DMSO-d₆) δ 7.96 (d, J=2.08 Hz, 1H), 7.53 (d, J=8.48 Hz, 1H), 7.46 (s, 1H), 7.13 (dd, J=1.52 Hz, 8.44 Hz, 1H), 6.92 (d, J=0.76 Hz, 1H), 3.83 (s, 2H), 2.12 (s, 3H). LCMS: Rt 1.74 min. MS (ES) C11H10O2 requires 174, found 175 [M+H]+.

### Step-2

To a stirred solution of 1-(benzofuran-5-yl)propan-2-one (9 g, 51.66 mmol, 1 eq.) in dry THF (150 ml) was added Ti(OEt)₄ (37.91 ml, 180.82 mmol, 3.5 eq.) and (R)-2-methylpropane-2-sulfinamide (6.26 g, 51.66 mmol, 1 eq.) (dissolved in 30 ml dry THF) and the resulting reaction mixture was allowed to stir at 70° C. for 12 hrs. Upon completion, monitored by TLC (50% EA in Hexane), the reaction mixture was cooled to 0° C., gradually to −48° C. and NaBH₄ (7.81 g, 206.65 mmol, 4 eq.) (dissolved in 30 ml dry THF) was added into the reaction mixture at −48° C. and the resulting reaction mixture was allowed to stir at −48° C. for 3 hrs. Upon completion, monitored by TLC (50% EA in Hexane), the reaction mixture was taken to room temperature and was quenched with Methanol and Sat NaCl solution (until white precipitate observed). The reaction mixture was then filtered through celite bed, washed with methanol (2×150 ml) and ethyl acetate (2×150 ml), evaporated under vacuum to remove the volatiles. Then the reaction mixture was extracted with ethyl acetate, washed with water, followed by brine solution. Combined organic layer was dried over anhydrous sodium sulphate, solvent was removed under vacuum to afford crude (R)-N-((R)-1-(benzofuran-5-yl)propan-2-yl)-2-methylpropane-2-sulfinamide as yellow sticky gum (14 g, 96%). ¹H NMR (400 MHz, DMSO-d₆) δ 7.94 (s, 1H), 7.48 (m, 2H), 7.15 (d, J=8.32 Hz, 1H), 6.89 (d, J=7.76 Hz, 1H), 4.97 (d, J=6.04 Hz, 1H), 3.48 (m, 1H), 3.07 (m, 1H), 2.76 (m, 1H), 1.09 (s, 12H), 1.08 (m, 3H) LCMS: Rt 1.87 min. MS (ES) C15H21NO2S requires 279, found 280 [M+H]+.

### Step-3

To a stirred solution of (R)-N-((R)-1-(benzofuran-5-yl)propan-2-yl)-2-methylpropane-2-sulfinamide (15 g, 53.57 mmol, 1 eq.) in dry THE (100 mL) (In a sealed tube) was added NaH (60%) (4.28 g, 107.14 mmol, 2 eq.) at 0° C. and the resulting reaction mixture was allowed to stir at 0° C. for 30 min. Then Iodomethane (6.7 ml, 107.14 mmol, 2 eq.) was added at 0° C. and the resulting reaction mixture was allowed to stir at room temperature for 12 h. Upon completion, monitored by TLC (50% EA in Hexane), the reaction mixture was quenched with ice water, extracted with ethyl acetate (2×250 ml), washed with saturated ammonium chloride solution, followed by brine solution. Combined organic layer was dried over anhydrous sodium sulphate, solvent was removed under vacuum and purified by silica gel column chromatography using ethyl acetate/hexane (50:50 v/v) as eluent to afford (R)-N-((R)-1-(benzofuran-5-yl)propan-2-yl)-N,2-dimethylpropane-2-sulfinamide as light-yellow gum (8 g, 50.9%). ¹H NMR (400 MHz, DMSO-d₆) δ 7.93 (s, 1H), 7.49 (m, 2H), 7.14 (d, J=7.4, 1H), 6.89 (s, 1H), 3.54 (m, 1H), 2.92 (m, 1H), 2.81 (m, 1H), 2.49 (s, 3H), 1.09 (d, J=6.64 Hz, 3H), 1.02 (s, 9H). LCMS: Rt 1.95 min. MS (ES) C16H23NO2S requires 293, found 294 [M+H]+.

### Step-4

To a stirred solution of (R)-N-((R)-1-(benzofuran-5-yl)propan-2-yl)-N,2-dimethylpropane-2-sulfinamide (10.5 g, 37.58 mmol, 1 eq.) in dry DCM (50 ml) was added 4M HCl in 1,4 dioxane (100 mL) at 0° C. and then the resulting reaction mixture was allowed to stir at room temperature for 2 h. Upon completion of reaction (monitored by TLC, 30% EA in Hexane), the solvent were evaporated and the crude was washed twice with diethyl ether (2×60 ml) and pentane and dried under vacuum to afford R-5-MAPB (5.8 g, 81%) as off white solid. ¹HNMR(400 MHz, DMSO-d₆) δ 9.00 (bs, 2H), 7.99 (d, J=1.6 Hz, 1H), 7.57 (m, 2H), 7.21 (d, J=7.8 Hz, 1H), 6.93 (s, 1H), 3.38 (bs, 1H), 3.25 (m, 1H), 2.77 (m, 1H), 2.56 (s, 3H), 1.11 (d, J=6.28 Hz, 3H). LCMS: Rt 1.32 min. MS (ES) C12H15NO requires 189, found 190 [M+H]+. HPLC: Rt 5.75 min. Purity (λ 210 nm): 99.26%.

### Preparation of aF-5-MAPB (1-(benzofuran-5-yl)-3-fluoro-N-methylpropan-2-amine)

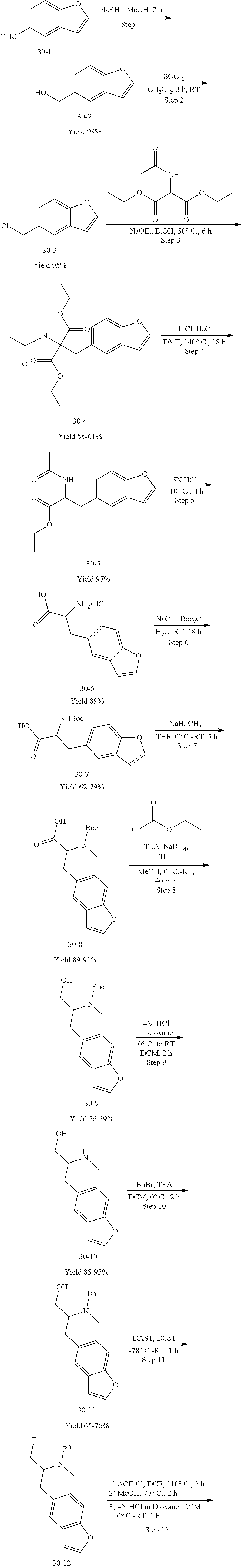

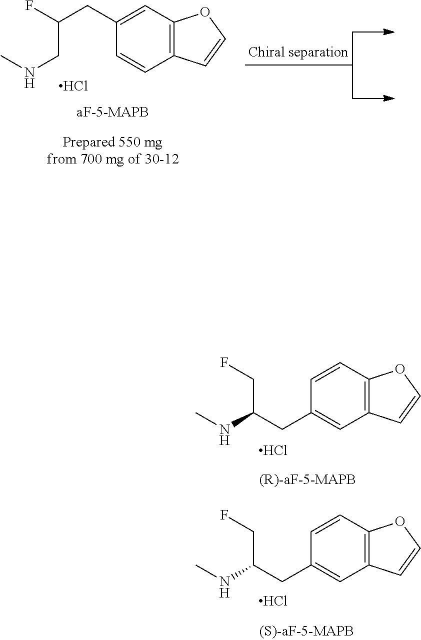

### Preparation of aF-6-MAPB (1-(benzofuran-6-yl)-3-fluoro-N-methylpropan-2-amine)

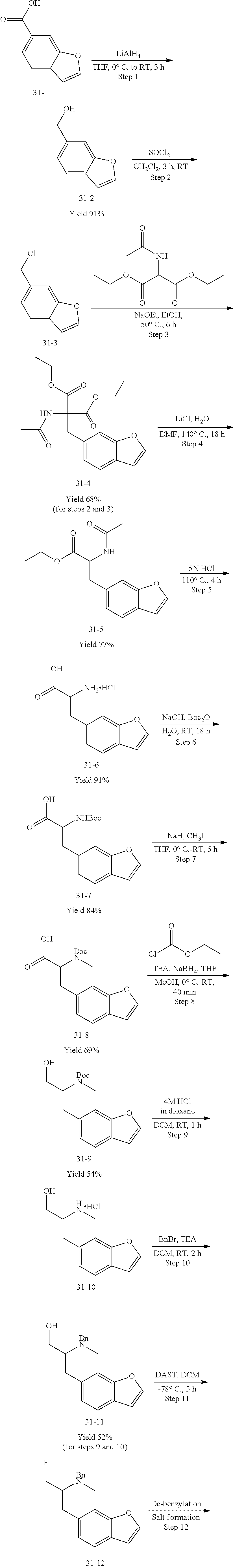

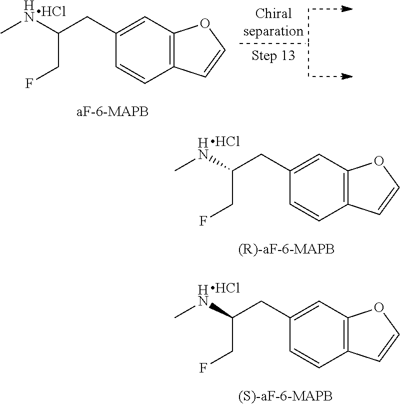

### Enantiomerically Enriched Pharmaceutical Compositions

Chiral compounds of the invention may be prepared by chiral chromatography from the racemic or enantiomerically enriched free amine or by chiral synthesis. Pharmaceutically acceptable salts of chiral compounds may be prepared from fractional crystallization of salts from a racemic or an enantiomerically enriched free amine and a chiral acid. Alternatively, the free amine may be reacted with a chiral auxiliary and the enantiomers separated by chromatography followed by removal of the chiral auxiliary to regenerate the free amine. Furthermore, separation of enantiomers may be performed at any convenient point in the synthesis of the compounds of the invention. Chirally pure material may be mixed at desired ratios to afford chirally enriched (for example enantiomerically enriched) mixtures.

An enantiomerically enriched mixture is a mixture that contains one enantiomer in a greater amount than the other. An enantiomerically enriched mixture of an S-enantiomer contains at least 55% of the S-enantiomer, and more typically at least about 60%, 65%, 70%, 75%, 80%, 85%, 90%, 95% of the S-enantiomer. An enantiomerically enriched mixture of an R-enantiomer contains at least 55% of the R-enantiomer, more typically at least about 55%, 60%, 65%, 70%, 75%, 80%, 85%, 90%, 95% of the R-enantiomer.

In one embodiment, enantiomerically enriched mixtures that have a greater amount of the R-enantiomer maximize nicotinic-receptor-dependent therapeutic effects. In one embodiment, enantiomerically enriched mixtures that have a greater amount of the S-enantiomer maximize serotonin-receptor-dependent therapeutic effects. Accordingly, in one embodiment, an enantiomerically enriched mixture of S-5-MAPB or an enantiomerically enriched mixture of S-6-MAPB maximize serotonin-receptor-dependent therapeutic effects and minimized unwanted nicotinic effects when administered to a host in need thereof, for example a mammal, including a human. In another embodiment, an enantiomerically enriched mixture of R-5-MAPB or an enantiomerically enriched mixture of R-6-MAPB maximize nicotinic-receptor-dependent therapeutic effects while minimizing unwanted effects, when administered to a host in need thereof, including a mammal, for example, a human.

Non-limiting examples of unwanted effects that can be minimized include psychoactive effects (such as excess stimulation or sedation), physiological effects (such as transient hypertension or appetite suppression), toxic effects (such as to the brain or liver), effects contributing to abuse liability (such as euphoria or dopamine release), and other side effects.

One aspect of the present invention is a balanced mixture of S-5-MAPB and R-5-MAPB (not the racemate) or a balanced mixture of S-6-MAPB and R-6-MAPB (not the racemate) that achieves a predetermined combination of serotonin-receptor-dependent therapeutic effects and nicotinic-receptor-dependent therapeutic effects.

In certain embodiments, pharmaceutical compositions of enantiomerically enriched preparations of 5-MAPB or 6-MAPB are provided. In one embodiment, the pharmaceutical composition is enriched with S-5-MAPB. In one embodiment, the pharmaceutical composition is enriched with R-5-MAPB. In one embodiment, the pharmaceutical composition is enriched with S-6-MAPB. In one embodiment, the pharmaceutical composition is enriched with R-6-MAPB.

Example 1 below provides a non-limiting example for the preparation of certain enantiomerically enriched preparations of 5-MAPB (i.e., comprising S-5-MAPB and R-5-MAPB). Enantiomerically enriched preparations of 6-MAPB (i.e., S-6-MAPB, R-6-MAPB) can be similarly produced using racemic 6-MAPB HCl.

Particular embodiments for pharmaceutical compositions, including enantiomerically enriched pharmaceutical compositions, of the present invention include:
a) S-5-MAPB;
b) R-5-MAPB;
c) S-6-MAPB;
d) R-6-MAPB;
e) Embodiments (a)-(d) wherein the compound is a free base;
f) Embodiments (a)-(d) wherein the compound is a salt;
g) Embodiment (f) wherein the compound is the hydrochloride salt;
h) A mixture of S-5-MAPB, R-5-MAPB and there is more S-enantiomer than R-enantiomer;
i) A mixture of S-5-MAPB, R-5-MAPB and there is less S-enantiomer than R-enantiomer;
j) A mixture of S-6-MAPB, R-6-MAPB and there is more S-enantiomer than R-enantiomer;
k) A mixture of S-6-MAPB, R-6-MAPB and there is less S-enantiomer than R-enantiomer;
l) A mixture of S-5-MAPB, R-5-MAPB and about 65% is the S-enantiomer while about 35% is the R-enantiomer;
m) A mixture of S-5-MAPB, R-5-MAPB and greater than 65% is the S-enantiomer while less than 35% is the R-enantiomer;
n) A mixture of S-5-MAPB, R-5-MAPB and greater than 90% is the S-enantiomer while less than 10% is the R-enantiomer;
o) A mixture of S-5-MAPB, R-5-MAPB and about 35% is the S-enantiomer while about 65% is the R-enantiomer;
p) A mixture of S-5-MAPB, R-5-MAPB and less than 35% is the S-enantiomer while greater than 65% is the R-enantiomer;
q) A mixture of S-5-MAPB, R-5-MAPB and less than 10% is the S-enantiomer while greater than 90% is the R-enantiomer;
r) A mixture of S-6-MAPB, R-6-MAPB and about 65% is the S-enantiomer while about 35% is the R-enantiomer;
s) A mixture of S-6-MAPB, R-6-MAPB and greater than 65% is the S-enantiomer while less than 35% is the R-enantiomer;
t) A mixture of S-6-MAPB, R-6-MAPB and greater than 90% is the S-enantiomer while less than 10% is the R-enantiomer;
u) A mixture of S-6-MAPB, R-6-MAPB and 35% or less is the S-enantiomer while 65% or more is the R-enantiomer;
v) A mixture of S-6-MAPB, R-6-MAPB and about 35% is the S-enantiomer while about 65% is the R-enantiomer; and
w) A mixture of S-6-MAPB, R-6-MAPB and less than 10% is the S-enantiomer while greater than 90% is the R-enantiomer.
x) S-5-MBPB;
y) R-5-MBPB;
z) S-6-MBPB;
aa) R-6-MBPB;
bb) Embodiments (x)-(aa) wherein the compound is a free base;
cc) Embodiments (x)-(aa) wherein the compound is a salt;
dd) Embodiment (cc) wherein the compound is the hydrochloride salt;
ee) A mixture of S-5-MBPB, R-5-MBPB and there is more S-enantiomer than R-enantiomer;
ff) A mixture of S-5-MBPB, R-5-MBPB and there is less S-enantiomer than R-enantiomer;
gg) A mixture of S-6-MBPB, R-6-MBPB and there is more S-enantiomer than R-enantiomer;
hh) A mixture of S-6-MBPB, R-6-MBPB and there is less S-enantiomer than R-enantiomer;
ii) A mixture of S-5-MBPB, R-5-MBPB and about 65% is the S-enantiomer while about 35% is the R-enantiomer;
jj) A mixture of S-5-MBPB, R-5-MBPB and greater than about 65% is the S-enantiomer while less than about 35% is the R-enantiomer;
kk) A mixture of S-5-MBPB, R-5-MBPB and greater than about 90% is the S-enantiomer while less than about 10% is the R-enantiomer;
ll) A mixture of S-5-MBPB, R-5-MBPB and about 35% is the S-enantiomer while about 65% is the R-enantiomer;
mm) A mixture of S-5-MBPB, R-5-MBPB and less than about 35% is the S-enantiomer while greater than about 65% is the R-enantiomer;
nn) A mixture of S-5-MBPB, R-5-MBPB and less than about 10% is the S-enantiomer while greater than about 90% is the R-enantiomer;
oo) A mixture of S-6-MBPB, R-6-MBPB and about 65% is the S-enantiomer while about 35% is the R-enantiomer;
pp) A mixture of S-6-MBPB, R-6-MBPB and greater than about 65% is the S-enantiomer while less than about 35% is the R-enantiomer;
qq) A mixture of S-6-MBPB, R-6-MBPB and greater than about 90% is the S-enantiomer while less than about 10% is the R-enantiomer;
rr) A mixture of S-6-MBPB, R-6-MBPB and about 35% or less is the S-enantiomer while about 65% or more is the R-enantiomer;
ss) A mixture of S-6-MBPB, R-6-MBPB and about 35% is the S-enantiomer while about 65% is the R-enantiomer; and
tt) A mixture of S-6-MBPB, R-6-MBPB and less than about 10% is the S-enantiomer while greater than about 90% is the R-enantiomer.
uu) S-Bk-5-MAPB;
vv) R-Bk-5-MAPB;
ww) S-Bk-6-MAPB;
xx) R-Bk-6-MAPB;
yy) Embodiments (uu)-(xx) wherein the compound is a free base;
zz) Embodiments (uu)-(xx) wherein the compound is a salt;
aaa) Embodiment (zz) wherein the compound is the hydrochloride salt;
bbb) A mixture of S-Bk-5-MAPB, R-Bk-5-MAPB and there is more S-enantiomer than R-enantiomer;
ccc) A mixture of S-Bk-5-MAPB, R-Bk-5-MAPB and there is less S-enantiomer than R-enantiomer;
ddd) A mixture of S-Bk-6-MAPB, R-Bk-6-MAPB and there is more S-enantiomer than R-enantiomer;
eee) A mixture of S-Bk-6-MAPB, R-Bk-6-MAPB and there is less S-enantiomer than R-enantiomer;
fff) A mixture of S-Bk-5-MAPB, R-Bk-5-MAPB and about 65% is the S-enantiomer while about 35% is the R-enantiomer;
ggg) A mixture of S-Bk-5-MAPB, R-Bk-5-MAPB and greater than about 65% is the S-enantiomer while less than about 35% is the R-enantiomer;
hhh) A mixture of S-Bk-5-MAPB, R-Bk-5-MAPB and greater than about 90% is the S-enantiomer while less than about 10% is the R-enantiomer;
iii) A mixture of S-Bk-5-MAPB, R-Bk-5-MAPB and about 35% is the S-enantiomer while about 65% is the R-enantiomer;
jjj) A mixture of S-Bk-5-MAPB, R-Bk-5-MAPB and less than about 35% is the S-enantiomer while greater than about 65% is the R-enantiomer;
kkk) A mixture of S-Bk-5-MAPB, R-Bk-5-MAPB and less than about 10% is the S-enantiomer while greater than about 90% is the R-enantiomer;
lll) A mixture of S-Bk-6-MAPB, R-Bk-6-MAPB and about 65% is the S-enantiomer while about 35% is the R-enantiomer;
mmm) A mixture of S-Bk-6-MAPB, R-Bk-6-MAPB and greater than about 65% is the S-enantiomer while less than about 35% is the R-enantiomer;
nnn) A mixture of S-Bk-6-MAPB, R-Bk-6-MAPB and greater than about 90% is the S-enantiomer while less than about 10% is the R-enantiomer;
ooo) A mixture of S-Bk-6-MAPB, R-Bk-6-MAPB and about 35% or less is the S-enantiomer while about 65% or more is the R-enantiomer;
ppp) A mixture of S-Bk-6-MAPB, R-Bk-6-MAPB and about 35% is the S-enantiomer while about 65% is the R-enantiomer; and
qqq) A mixture of S-Bk-6-MAPB, R-Bk-6-MAPB and less than about 10% is the S-enantiomer while greater than about 90% is the R-enantiomer.
rrr) S-Bk-5-MBPB;
sss) R-Bk-5-MBPB;
ttt) S-Bk-6-MBPB;
uuu) R-Bk-6-MBPB;
vvv) Embodiments (rrr)-(uuu) wherein the compound is a free base;
www) Embodiments (rrr)-(uuu) wherein the compound is a salt;
xxx) Embodiment (www) wherein the compound is the hydrochloride salt;
yyy) A mixture of S-Bk-5-MBPB, R-Bk-5-MBPB and there is more S-enantiomer than R-enantiomer;
zzz) A mixture of S-Bk-5-MBPB, R-Bk-5-MBPB and there is less S-enantiomer than R-enantiomer;
aaaa) A mixture of S-Bk-6-MBPB, R-Bk-6-MBPB and there is more S-enantiomer than R-enantiomer;
bbbb) A mixture of S-Bk-6-MBPB, R-Bk-6-MBPB and there is less S-enantiomer than R-enantiomer;
cccc) A mixture of S-Bk-5-MBPB, R-Bk-5-MBPB and about 65% is the S-enantiomer while about 35% is the R-enantiomer;
dddd) A mixture of S-Bk-5-MBPB, R-Bk-5-MBPB and greater than about 65% is the S-enantiomer while less than about 35% is the R-enantiomer;
eeee) A mixture of S-Bk-5-MBPB, R-Bk-5-MBPB and greater than about 90% is the S-enantiomer while less than about 10% is the R-enantiomer;
ffff) A mixture of S-Bk-5-MBPB, R-Bk-5-MBPB and about 35% is the S-enantiomer while about 65% is the R-enantiomer;
gggg) A mixture of S-Bk-5-MBPB, R-Bk-5-MBPB and less than about 35% is the S-enantiomer while greater than about 65% is the R-enantiomer;
hhhh) A mixture of S-Bk-5-MBPB, R-Bk-5-MBPB and less than about 10% is the S-enantiomer while greater than about 90% is the R-enantiomer;
iiii) A mixture of S-Bk-6-MBPB, R-Bk-6-MBPB and about 65% is the S-enantiomer while about 35% is the R-enantiomer;
jjjj) A mixture of S-Bk-6-MBPB, R-Bk-6-MBPB and greater than about 65% is the S-enantiomer while less than about 35% is the R-enantiomer;
kkkk) A mixture of S-Bk-6-MBPB, R-Bk-6-MBPB and greater than about 90% is the S-enantiomer while less than about 10% is the R-enantiomer;
llll) A mixture of S-Bk-6-MBPB, R-Bk-6-MBPB and about 35% or less is the S-enantiomer while about 65% or more is the R-enantiomer;
mmmm) A mixture of S-Bk-6-MBPB, R-Bk-6-MBPB and about 35% is the S-enantiomer while about 65% is the R-enantiomer; and
nnnn) A mixture of S-Bk-6-MBPB, R-Bk-6-MBPB and less than about 10% is the S-enantiomer while greater than about 90% is the R-enantiomer.
oooo) R-BK-5-EAPB;
pppp) S-BK-5-EAPB;
qqqq) S-BK-6-EAPB;
rrrr) R-BK-6-EAPB;
ssss) Embodiments (oooo)-(rrrr) wherein the compound is a free base;
tttt) Embodiments (oooo)-(rrrr) wherein the compound is a salt;
uuuu) Embodiment (tttt) wherein the compound is the hydrochloride salt;
vvvv) A mixture of S-BK-5-EAPB, R-BK-5-EAPB and there is more S-enantiomer than R-enantiomer;
wwww) A mixture of S-BK-5-EAPB, R-BK-5-EAPB and there is less S-enantiomer than R-enantiomer;
xxxx) A mixture of S-BK-6-EAPB, R-BK-6-EAPB and there is more S-enantiomer than R-enantiomer;
yyyy) A mixture of S-BK-6-EAPB, R-BK-6-EAPB and there is less S-enantiomer than R-enantiomer;
zzzz) A mixture of S-BK-5-EAPB, R-BK-5-EAPB and about 65% is the S-enantiomer while about 35% is the R-enantiomer;
aaaaa) A mixture of S-BK-5-EAPB, R-BK-5-EAPB and greater than 65% is the S-enantiomer while less than 35% is the R-enantiomer;
bbbbb) A mixture of S-BK-5-EAPB, R-BK-5-EAPB and greater than 90% is the S-enantiomer while less than 10% is the R-enantiomer;
ccccc) A mixture of S-BK-5-EAPB, R-BK-5-EAPB and about 35% is the S-enantiomer while about 65% is the R-enantiomer;
ddddd) A mixture of S-BK-5-EAPB, R-BK-5-EAPB and less than 35% is the S-enantiomer while greater than 65% is the R-enantiomer;
eeeee) A mixture of S-BK-5-EAPB, R-BK-5-EAPB and less than 10% is the S-enantiomer while greater than 90% is the R-enantiomer;
fffff) A mixture of S-BK-6-EAPB, R-BK-6-EAPB and about 65% is the S-enantiomer while about 35% is the R-enantiomer;
ggggg) A mixture of S-BK-6-EAPB, R-BK-6-EAPB and greater than 65% is the S-enantiomer while less than 35% is the R-enantiomer;
hhhhh) A mixture of S-BK-6-EAPB, R-BK-6-EAPB and greater than 90% is the S-enantiomer while less than 10% is the R-enantiomer;
iiiii) A mixture of S-6 BK-6-EAPB, R-BK-6-EAPB and 35% or less is the S-enantiomer while 65% or more is the R-enantiomer;
jjjjj) A mixture of S-BK-6-EAPB, R-BK-6-EAPB and about 35% is the S-enantiomer while about 65% is the R-enantiomer; and
kkkkk) A mixture of S-BK-6-EAPB, R-BK-6-EAPB and less than 10% is the S-enantiomer while greater than 90% is the R-enantiomer.
lllll) R-BK-5-EBPB;
mmmmm) S-BK-5-EBPB;
nnnnn) S-BK-6-EBPB;
ooooo) R-BK-6-EBPB;
ppppp) Embodiments (lllll)-(ooooo) wherein the compound is a free base;
qqqqq) Embodiments (lllll)-(ooooo) wherein the compound is a salt;
rrrrr) Embodiment (qqqqq) wherein the compound is the hydrochloride salt;
sssss) A mixture of S-BK-5-EBPB, R-BK-5-EBPB and there is more S-enantiomer than R-enantiomer;
ttttt) A mixture of S-BK-5-EBPB, R-BK-5-EBPB and there is less S-enantiomer than R-enantiomer;
uuuuu) A mixture of S-BK-6-EBPB, R-BK-6-EBPB and there is more S-enantiomer than R-enantiomer;
vvvvv) A mixture of S-BK-6-EBPB, R-BK-6-EBPB and there is less S-enantiomer than R-enantiomer;
wwwww) A mixture of S-BK-5-EBPB, R-BK-5-EBPB and about 65% is the S-enantiomer while about 35% is the R-enantiomer;
xxxxx) A mixture of S-BK-5-EBPB, R-BK-5-EBPB and greater than 65% is the S-enantiomer while less than 35% is the R-enantiomer;
yyyyy) A mixture of S-BK-5-EBPB, R-BK-5-EBPB and greater than 90% is the S-enantiomer while less than 10% is the R-enantiomer;
zzzzz) A mixture of S-BK-5-EBPB, R-BK-5-EBPB and about 35% is the S-enantiomer while about 65% is the R-enantiomer;
aaaaaa) A mixture of S-BK-5-EBPB, R-BK-5-EBPB and less than 35% is the S-enantiomer while greater than 65% is the R-enantiomer;
bbbbbb) A mixture of S-BK-5-EBPB, R-BK-5-EBPB and less than 10% is the S-enantiomer while greater than 90% is the R-enantiomer;
cccccc) A mixture of S-BK-6-EBPB, R-BK-6-EBPB and about 65% is the S-enantiomer while about 35% is the R-enantiomer;
dddddd) A mixture of S-BK-6-EBPB, R-BK-6-EBPB and greater than 65% is the S-enantiomer while less than 35% is the R-enantiomer;
eeeeee) A mixture of S-BK-6-EBPB, R-BK-6-EBPB and greater than 90% is the S-enantiomer while less than 10% is the R-enantiomer;
ffffff) A mixture of S-6 BK-6-EBPB, R-BK-6-EBPB and 35% or less is the S-enantiomer while 65% or more is the R-enantiomer;
gggggg) A mixture of S-BK-6-EBPB, R-BK-6-EBPB and about 35% is the S-enantiomer while about 65% is the R-enantiomer; and
hhhhhh) A mixture of S-BK-6-EBPB, R-BK-6-EBPB and less than 10% is the S-enantiomer while greater than 90% is the R-enantiomer.
iiiiii) S-aF-5-MAPB;
jjjjjj) R-aF-5-MAPB;
kkkkkk) S-aF-6-MAPB;
llllll) R-aF-6-MAPB;
mmmmmm) Embodiments (irmi)-(llllll) wherein the compound is a free base;
nnnnnn) Embodiments (irmi)-(llllll) wherein the compound is a salt;
oooooo) Embodiment (nnnnnn) wherein the compound is the hydrochloride salt;
pppppp) A mixture of S-aF-5-MAPB, R-aF-5-MAPBand there is more S-enantiomer than R-enantiomer;
qqqqqq) A mixture of S-aF-5-MAPB, R-aF-5-MAPBand there is less S-enantiomer than R-enantiomer;
rrrrrr) A mixture of S-aF-6-MAPB, R-aF-6-MAPB and there is more S-enantiomer than R-enantiomer;
ssssss) A mixture of S-aF-6-MAPB, R-aF-6-MAPB and there is less S-enantiomer than R-enantiomer;
tttttt) A mixture of S-aF-5-MAPB, R-aF-5-MAPBand about 65% is the S-enantiomer while about 35% is the R-enantiomer;
uuuuuu) A mixture of S-aF-5-MAPB, R-aF-5-MAPB and greater than about 65% is the S-enantiomer while less than about 35% is the R-enantiomer;
vvvvvv) A mixture of S-aF-5-MAPB, R-aF-5-MAPBand greater than about 90% is the S-enantiomer while less than about 10% is the R-enantiomer;
wwwwww) A mixture of S-aF-5-MAPB, R-aF-5-MAPBand about 35% is the S-enantiomer while about 65% is the R-enantiomer;
xxxxxx) A mixture of S-aF-5-MAPB, R-aF-5-MAPBand less than about 35% is the S-enantiomer while greater than about 65% is the R-enantiomer;
yyyyyy) A mixture of S-aF-5-MAPB, R-aF-5-MAPBand less than about 10% is the S-enantiomer while greater than about 90% is the R-enantiomer;
zzzzzz) A mixture of S-aF-6-MAPB, R-aF-6-MAPB and about 65% is the S-enantiomer while about 35% is the R-enantiomer;
aaaaaaa) A mixture of S-aF-6-MAPB, R-aF-6-MAPB and greater than about 65% is the S-enantiomer while less than about 35% is the R-enantiomer;
bbbbbbb) A mixture of S-aF-6-MAPB, R-aF-6-MAPB and greater than about 90% is the S-enantiomer while less than about 10% is the R-enantiomer;
ccccccc) A mixture of S-aF-6-MAPB, R-aF-6-MAPB and about 35% or less is the S-enantiomer while about 65% or more is the R-enantiomer;
ddddddd) A mixture of S-aF-6-MAPB, R-aF-6-MAPB and about 35% is the S-enantiomer while about 65% is the R-enantiomer; and
eeeeeee) A mixture of S-aF-6-MAPB, R-aF-6-MAPB and less than about 10% is the S-enantiomer while greater than about 90% is the R-enantiomer.

It will be understood that the above embodiments and classes of embodiments can be combined to form additional preferred embodiments.

### ADDITIONAL EMBODIMENTS

1. In certain embodiments a method is provided for treating a central nervous system disorder comprising administering an effective amount of two therapeutic agents wherein one therapeutic agent is an entactogenic compound or a pharmaceutically acceptable salt or salt mixture thereof and the other is a dopamine releasing agent a pharmaceutically acceptable salt or salt mixture thereof, wherein the two agents are administered to produce a kinetic lag between their therapeutic effects, wherein the entactogenic compound has a serotonin release EC₅₀ of less than 10 μM and the dopamine releasing agent has a dopamine release EC₅₀ of less than 10 μM, and wherein the kinetic lag is characterized by one of the agents having a more rapid onset of therapeutic effects than the other.
2. The method of embodiment 1, wherein the entactogenic compound has a serotonin release EC₅₀ of less than 1 μM.
3. The method of embodiment 1, wherein the entactogenic compound has a serotonin release EC₅₀ of less than 250 nM.
4. The method of any one of embodiments 1-3, wherein the dopamine releasing agent has a dopamine release EC₅₀ of less than 1 μM.
5. The method of any one of embodiments 1-3, wherein the dopamine releasing agent has a dopamine release EC₅₀ of less than 250 nM.
6. The method of any one of embodiments 1-5, wherein the dopamine releasing agent has a DAT to SERT EC₅₀ ratio that is at least two times greater than the than the DAT to SERT EC₅₀ ratio for the entactogenic compound.
7. The method of any one of embodiments 1-5, wherein the dopamine releasing agent has a DAT to SERT EC₅₀ ratio that is at least three times greater than the than the DAT to SERT EC₅₀ ratio for the entactogenic compound.
8. The method of any one of embodiments 1-5, wherein the dopamine releasing agent has a DAT to SERT EC₅₀ ratio that is at least four times greater than the than the DAT to SERT EC₅₀ ratio for the entactogenic compound.
9. The method of any one of embodiments 1-5, wherein the dopamine releasing agent has a DAT to SERT EC₅₀ ratio that is at least five times greater than the than the DAT to SERT EC₅₀ ratio for the entactogenic compound.
10. The method of any one of embodiments 1-5, wherein the dopamine releasing agent has a DAT to SERT EC₅₀ ratio that is at least ten times greater than the than the DAT to SERT EC₅₀ ratio for the entactogenic compound.
11. The method of any one of embodiments 1-10, wherein the entactogenic compound is selected from:

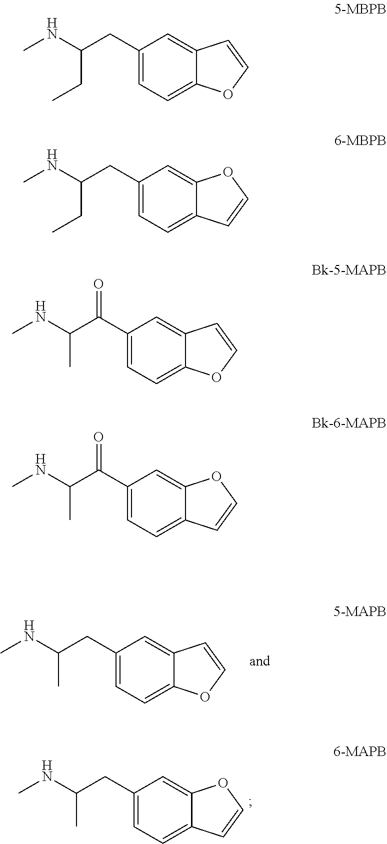

or a salt or salt mixture thereof, optionally as an enantiomerically pure or enantiomerically enriched mixture.
12. The method of any one of embodiments 1-11, wherein the entactogenic compound is

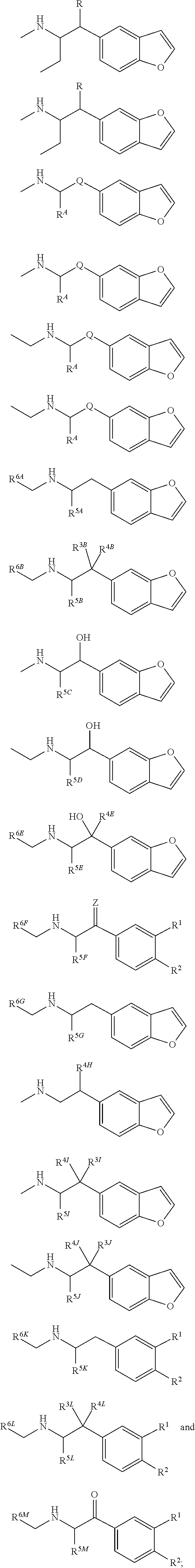

or a salt or salt mixture thereof, optionally as an enantiomerically pure or enantiomerically enriched mixture;
wherein:
R is hydrogen or hydroxyl;
R^{A} is —CH₃, —CH₂Y, —CHY₂, —CY₃, —CH₂CH₃, —CH₂CH₂Y, —CH₂CHY₂, —CH₂CY₃, —CH₂OH, or —CH₂CH₂OH;
Q is selected from;

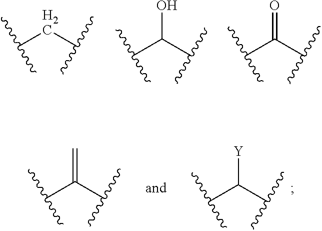

Y is halogen;
R¹ and R² are taken together as —OCH═CH— or —CH═CHO—;
R^{3B} and R^{4B} are independently selected from —H, —X, C₁-C₄ alkyl, —CH₂OH, —CH₂X, —CHX₂, and —CX₃, wherein at least one of R^{3B} and R^{4B} is not —H;
R^{3L} and R^{4L} are independently selected from —H, —X, —OH, C₁-C₄ alkyl, —CH₂OH, —CH₂X, —CHX₂, and —CX₃, wherein at least one of R^{3L} and R^{4L} is not —H;
R^{3I} and R^{4I} are independently selected from —H, —X, —OH, —CH₂OH, —CH₂X, —CHX₂, —CX₃, and C₁-C₄ alkyl; wherein at least one of R^{3I} and R^{4I} is not —H;
R^{3J} and R^{4J} are independently selected from —H, —X, —OH, C₁-C₄ alkyl, —CH₂OH, —CH₂X, —CHX₂, and —CX₃;
R^{4E} is selected from C₁-C₄ alkyl, —CH₂OH, —CH₂X, —CHX₂, and —CX₃;
R^{4H} is selected from —X, —CH₂CH₂CH₃, —CH₂OH, —CH₂X, and —CHX₂;
R^{5A} and R^{5G} are independently selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₂-C₄ alkyl, when R^{5A} is C₂ alkyl or H, R^{6A} is not —H, and when R^{5G} is —H or C₂ alkyl, R^{6G} is not —H;
R^{5B} is selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₁-C₄ alkyl;
R^{5C} is selected from —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₂-C₄ alkyl;
R^{5D}, R^{5E}, R^{5F}, and R^{5J} are independently selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₁-C₄ alkyl, when R^{5F} is —H or C₁ alkyl, R^{6F} cannot be —H, and when R^{5J} is C₁ alkyl, at least one of R^{3J} and R^{4J} is not H;
R^{5K} is selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₂-C₄ alkyl;
R^{5L} and R^{5M} are independently selected from —H, —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₁-C₄ alkyl; and
R^{5I} is selected from —CH₂OH, —CH₂X, —CHX₂, —CX₃, —CH₂CH₂OH, —CH₂CH₂X, —CH₂CHX₂, —CH₂CX₃, C₃-C₄ cycloalkyl, and C₁-C₄ alkyl; wherein at least one of R^{3I}, R^{4I}, and R^{5I} is not C₁ alkyl;
R^{6A}, R^{6B}, R^{6E}, R^{6F}, and R^{6G} are independently selected from —H and —CH₃;
R^{6K}, R^{6L}, and R^{6M} are independently selected from —H and —CH₃;
X is independently selected from —F, —Cl, and —Br; and
Z is selected from O and CH_{2.}
13. The method of any one of embodiments 1-11, wherein the entactogenic compound is

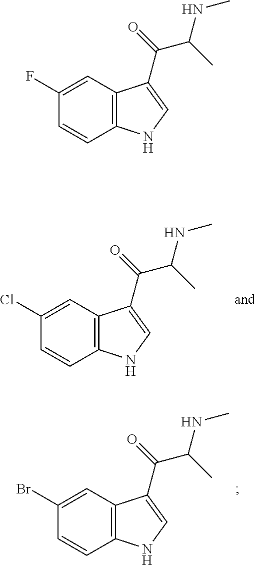 [SMILES: CNC(C)C(=O)c1c[nH]c2ccc(F)cc12 ; CNC(C)C(=O)c1c[nH]c2ccc(Cl)cc12 ; CNC(C)C(=O)c1c[nH]c2ccc(Br)cc12]

or a salt or salt mixture thereof, optionally as an enantiomerically pure or enantiomerically enriched mixture.
14. The method of any one of embodiments 1-11, wherein the entactogenic compound is

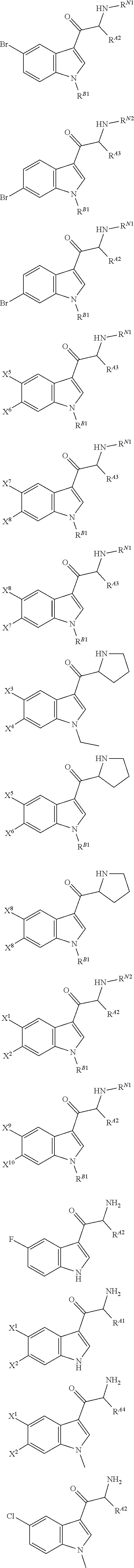

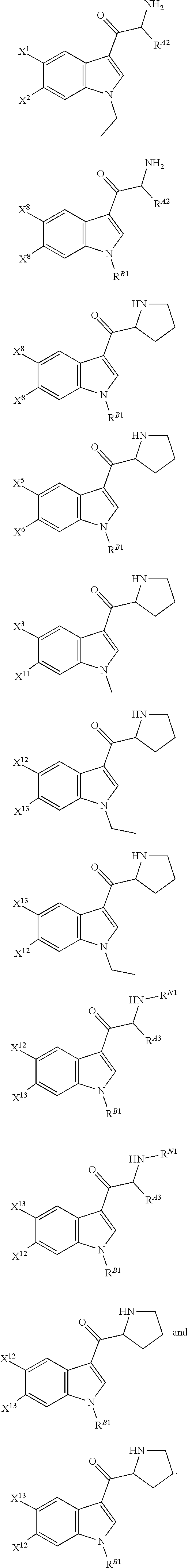

15. The method of any one of embodiments 1-11, wherein the entactogenic compound is selected from: MDMA, MDA (3,4-methylenedioxyamphetamine), MDAI (2H,5H,6H,7H-indeno[5,6-d][1,3]dioxol-6-amine), BK-MDMA (1-(2H-1,3-benzodioxol-5-yl)-2-(methylamino)propan-1-one), BK-MDEA (1-(2H-1,3-benzodioxol-5-yl)-2-(ethylamino)propan-1-one), MBDB ([1-(2H-1,3-benzodioxol-5-yl)butan-2-yl](methyl)amine), butylone (1-(2H-1,3-benzodioxol-5-yl)-2-(methylamino)butan-1-one), eutylone (1-(2H-1,3-benzodioxol-5-yl)-2-(ethylamino)butan-1-one), MDEA ([1-(2H-1,3-benzodioxol-5-yl)propan-2-yl](ethyl)amine), αMT (1-(1H-indol-3-yl)propan-2-amine), α,N-DMT ([1-(1H-indol-3-yl)propan-2-yl](methyl)amine), BK-NM-AMT (1-(1H-indol-3-yl)-2-(methylamino)propan-1-one), 5-F-NM-AMT ([1-(5-fluoro-1H-indol-3-yl)propan-2-yl](methyl)amine), BK-5-F-NM-AMT (1-(5-fluoro-1H-indol-3-yl)-2-(methylamino)propan-1-one), 2-APB (1-(1-benzofuran-2-yl)propan-2-amine), 5-APB (1-(1-benzofuran-5-yl)propan-2-amine), 6-APB ((1-(1-benzofuran-6-yl)propan-2-amine), APBI (5H,6H,7H-indeno[5,6-b]furan-6-amine), 2-MAPB ([1-(1-benzofuran-2-yl)propan-2-yl](methyl)amine), 5-MAPB ([1-(1-benzofuran-5-yl)propan-2-yl](methyl)amine), 6-MAPB ([1-(1-benzofuran-6-yl)propan-2-yl](methyl)amine), 2-EAPB ([1-(1-benzofuran-2-yl)propan-2-yl](ethyl)amine), 5-EAPB ([1-(1-benzofuran-2-yl)propan-5-yl](ethyl)amine), 6-EAPB ([1-(1-benzofuran-6-yl)propan-2-yl](ethyl)amine), 2-MBPB ([1-(1-benzofuran-2-yl)butan-2-yl](methyl)amine), 5-MBPB ([1-(1-benzofuran-5-yl)butan-2-yl](methyl)amine), 6-MBPB ([1-(1-benzofuran-6-yl)butan-2-yl](methyl)amine), BK-5-MAPB (1-(1-benzofuran-5-yl)-2-(methylamino)propan-1-one), BK-2-MAPB (1-(1-benzofuran-2-yl)-2-(methylamino)propan-1-one), FLEA (N-[1-(2H-1,3-benzodioxol-5-yl)propan-2-yl]-N-methylhydroxylamine), and MDOH (N-[1-(2H-1,3-benzodioxol-5-yl)propan-2-yl]hydroxylamine).
16. The method of any one of embodiments 1-15, wherein the dopamine releasing agent is selected from amphetamine, fencamfamine, phenmetrazine, 2-fluorophenmetrazine, 3-fluorophenmetrazine, metamnetamine (methyl[1-(5,6,7,8-tetrahydronaphthalen-2-yl)propan-2-yl]amine), naphthylaminopropane, 5-(2-Aminopropyl)indole, methcathinone, 2-methyl-methcathinone, 3-methyl-methcathinone, 4-methyl-methcathinone (4-MMC), 3-fluoroamphetamine, 3-fluoromethcathinone, 4-fluoroamphetamine, 4-fluoromethcathinone, 3-bromoamphetamine, 3-bromomethcathinone, 4-bromoamphetamine, 4-bromomethcathinone, N-methylamphetamine, N-benzyl-methamphetamine, 3-methylamphetamine, 4-methylamphetamine, N,4-dimethylamphetamine, 2-(Methylamino)-1-naphthalen-1-ylpropan-1-one, methylthioamphetamine, and N,N-dimethyl-thioamphetamine.
17. The method of any one of embodiments 1-16, wherein the dopamine releasing agent has a lower DAT EC₅₀ than the entactogenic compound.
18. The method of any one of embodiments 1-16, wherein the entactogenic compound has a two times greater DAT EC₅₀ than the dopamine releasing agent.
19. The method of any one of embodiments 1-16, wherein the entactogenic compound has a three times greater DAT EC₅₀ than the dopamine releasing agent.
20. The method of any one of embodiments 1-16, wherein the entactogenic compound has a four times greater DAT EC₅₀ than the dopamine releasing agent.
21. The method of any one of embodiments 1-16, wherein the entactogenic compound has a five times greater DAT EC₅₀ than the dopamine releasing agent.
22. The method of any one of embodiments 1-21, wherein the kinetic lag is a difference in Tmax of the agents of more than about 5, 10, 15, 20, 25, 30, 35, 40, 45, 50, 55, 60, 90, or 120.
23. The method of any one of embodiments 1-21, wherein the kinetic lag is a difference in timing of the 50% Cmax of the agents of more than about 5, 10, 15, 20, 25, 30, 35, 40, 45, 50, 55, 60, 90, or 120 minutes.
24. The method of any one of embodiments 1-23, wherein the entactogenic compound and dopamine releasing agent are in the same dosage form and have different release profiles.
25. The method of any one of embodiments 1-23, wherein the entactogen and dopamine releasing agents are in the different dosage forms and one dosage form uses parenteral administration.
26. The method of embodiment 25, wherein the dosage forms are oral and inhaled.
27. The method of embodiment 25, wherein the dosage forms are oral and injected.
28. The method of embodiment 27, wherein the injection is intravenous.
29. The method of embodiment 27, wherein the injection is intramuscular.
30. The method of embodiment 25, wherein the dosage forms are oral and buccal or sublingual.
31. The method of any one of embodiments 1 or 17-30 wherein the entactogenic compound is MDMA and the dopamine releasing agents are one or more of amphetamine, and methamphetamine, or prodrugs of the same.
32. The method of any one of embodiments 1 or 17-30 wherein the entactogenic compound is 5-MAPB and the dopamine releasing agents are one or more of amphetamine, and methamphetamine, or prodrugs of the same.
33. The method of any one of embodiments 1 or 17-30 wherein the entactogenic compound is MDMA and the dopamine releasing agent is BK-MDMA.
34. The method of any one of embodiments 1 or 17-30 wherein the entactogenic compound is enantiomerically enriched 6-MBPB (with more S- than R-enantiomer) and the dopamine releasing agent is enantiomerically enriched 6-MBPB (with more R- than S-enantiomer).
35. The method of any one of embodiments 1 or 17-30 wherein the entactogenic compound is enantiomerically enriched 5-MBPB (with more S- than R-enantiomer) and the dopamine releasing agent is enantiomerically enriched 5-MBPB (with more R- than S-enantiomer).
36. The method of any one of embodiments 1 or 17-30 wherein the entactogenic compound is R-5-MAPB and the dopamine releasing agent is S-5-MAPB.
37. The method of any one of embodiments 1 or 17-30 wherein the entactogenic compound is 5-MAPB and the dopamine releasing agent is amphetamine, and methamphetamine, or prodrugs of the same.
38. The method of any one of embodiments 1 or 17-30 wherein the entactogenic compound is MDMA and the dopamine releasing agent is 4-MMC.
39. The method of any one of embodiments 1 or 17-30 wherein the entactogenic compound is S-4-MMC and the dopamine releasing agent is R-4-MMC.
40. The method of any one of embodiments 1 or 17-30 wherein the entactogenic compound is R-MDA and the dopamine releasing agent is S-MDA.
41. The method of any one of embodiments 1-40 wherein the method decreases an undesired effect selected from nausea, vomiting, anxiety, dizziness, sedation, drunkenness, difficulty concentrating, and mild confusion as compared to the administration of the entactogenic compound alone.
42. The method of any one of embodiments 1-41 wherein the method increases a desired effect selected from neuroticism, increased authenticity, and positive mood as compared to the administration of the entactogenic compound alone.
43. The method of any one of embodiments 1-42 wherein the central nervous system disorder is selected from: post-traumatic stress disorder, depression, dysthymia, anxiety, generalized anxiety, social anxiety, panic, adjustment disorder, feeding and eating disorders, binge behaviors, body dysmorphic syndromes, addiction, drug abuse or dependence disorders, substance use disorders, disruptive behavior disorders, impulse control disorders, gaming disorders, gambling disorders, memory loss, dementia of aging, attention deficit hyperactivity disorder, personality disorders, attachment disorders, autism and dissociative disorders.
44. The method of any one of embodiments 1-43 wherein the central nervous system disorder is post-traumatic stress disorder.
45. The method of any one of embodiments 1-43 wherein the central nervous system disorder is adjustment disorder.
46. The method of any one of embodiments 1-43 wherein the central nervous system disorder is generalized anxiety.
47. The method of any one of embodiments 1-43 wherein the central nervous system disorder is social anxiety.
48. The method of any one of embodiments 1-43 wherein the central nervous system disorder is depression.
49. The method of any one of embodiments 1-43 wherein the central nervous system disorder is a substance use disorder.
50. The method of any one of embodiments 1-43 wherein the central nervous system disorder is an attachment disorder.
51. The method of any one of embodiments 1-43 wherein the central nervous system disorder is schizophrenia.
52. The method of any one of embodiments 1-43 wherein the central nervous system disorder is an eating disorder.
53. The method of embodiment 52 wherein the eating disorder is bulimia.
54. The method of embodiment 52 wherein the eating disorder is binge eating.
55. The method of embodiment 52 wherein the eating disorder is anorexia.
56. The method of any one of embodiments 1-55 wherein there are multiple central nervous system disorders.
57. The method of any one of embodiments 1-55 wherein the central nervous system disorder is a neurological disorder.
58. The method of embodiment 57 wherein the neurological disorder is stroke.
59. The method of embodiment 57 wherein the neurological disorder is brain trauma.
60. The method of embodiment 57 wherein the neurological disorder is dementia.
61. The method of embodiment 57 wherein the neurological disorder is a neurodegenerative disease or disorder.
62. The method of embodiment 61 wherein the neurodegenerative disease or disorder is selected from: Alzheimer's disease, mild cognitive impairment (MCI), Parkinson's disease, Parkinson's disease dementia, multiple sclerosis, adrenoleukodystrophy, AIDS dementia complex, Alexander disease, Alper's disease, amyotrophic lateral sclerosis (ALS), ataxia telangiectasia, Batten disease, bovine spongiform encephalopathy, Canavan disease, cerebral amyloid angiopathy, cerebellar ataxia, Cockayne syndrome, corticobasal degeneration, Creutzfeldt-Jakob disease, diffuse myelinoclastic sclerosis, fatal familial insomnia, Fazio-Londe disease, Friedreich's ataxia, frontotemporal dementia or lobar degeneration, hereditary spastic paraplegia, Huntington disease, Kennedy's disease, Krabbe disease, Lewy body dementia, Lyme disease, Machado-Joseph disease, motor neuron disease, Multiple systems atrophy, neuroacanthocytosis, Niemann-Pick disease, Pelizaeus-Merzbacher Disease, Pick's disease, primary lateral sclerosis including its juvenile form, progressive bulbar palsy, progressive supranuclear palsy, Refsum's disease including its infantile form, Sandhoff disease, Schilder's disease, spinal muscular atrophy, spinocerebellar ataxia, Steele-Richardson-Olszewski disease, subacute combined degeneration of the spinal cord, survival motor neuron spinal muscular atrophy, Tabes dorsalis, Tay-Sachs disease, toxic encephalopathy, transmissible spongiform encephalopathy, Vascular dementia, X-linked spinal muscular atrophy, synucleinopathy, progranulinopathy, tauopathy, amyloid disease, prion disease, protein aggregation disease, and movement disorder.
63. The method of any one of embodiments 1-62 wherein the entactogenic compound and dopamine releasing agent are administered in a clinical setting.
64. The method of any one of embodiments 1-62 wherein the entactogenic compound and dopamine releasing agent are administered in an at-home setting.
65. The method of any one of embodiments 1-62 wherein the entactogenic compound and dopamine releasing agent are administered during a psychotherapy session.
66. The method of any one of embodiments 1-62 wherein the entactogenic compound and dopamine releasing agent are administered during a counseling session.
67. The method of any one of embodiments 1-66, wherein the mixture of entactogenic compound and dopamine releasing agent is described in Table 5.
68. The method of any one of embodiments 1-66, wherein the mixture of entactogenic compound and dopamine releasing agent is described in Table 6.
69. The method of any one of embodiments 1-66, wherein the mixture of entactogenic compound and dopamine releasing agent is described in Table 7.
70. The method of any one of embodiments 1-66, wherein the mixture of entactogenic compound and dopamine releasing agent is described in Table 8.
71. A combination of two therapeutic agents wherein one therapeutic agent is an entactogenic compound or a pharmaceutically acceptable salt or salt mixture thereof and the other is a dopamine releasing agent a pharmaceutically acceptable salt or salt mixture thereof, for treating a central nervous system disorder, wherein the two agents are administered to produce a kinetic lag between their therapeutic effects, wherein the entactogenic compound has a serotonin release EC₅₀ of less than 10 μM and the dopamine releasing agent has a dopamine release EC₅₀ of less than 10 μM, and wherein the kinetic lag is characterized by one of the agents having a more rapid onset of therapeutic effects than the other.
72. In certain embodiments the se of a combination of two therapeutic agents is provided wherein one therapeutic agent is an entactogenic compound or a pharmaceutically acceptable salt or salt mixture thereof and the other is a dopamine releasing agent a pharmaceutically acceptable salt or salt mixture thereof, for treating a central nervous system disorder, wherein the two agents are administered to produce a kinetic lag between their therapeutic effects, wherein the entactogenic compound has a serotonin release EC₅₀ of less than 10 μM and the dopamine releasing agent has a dopamine release EC₅₀ of less than 10 μM, and wherein the kinetic lag is characterized by one of the agents having a more rapid onset of therapeutic effects than the other.
73. In certain embodiments the use of two therapeutic agents in the manufacture of a medicament is provided wherein one therapeutic agent is an entactogenic compound or a pharmaceutically acceptable salt or salt mixture thereof and the other is a dopamine releasing agent a pharmaceutically acceptable salt or salt mixture thereof, for treating a central nervous system disorder, wherein the two agents are administered to produce a kinetic lag between their therapeutic effects, wherein the entactogenic compound has a serotonin release EC₅₀ of less than 10 μM and the dopamine releasing agent has a dopamine release EC₅₀ of less than 10 μM, and wherein the kinetic lag is characterized by one of the agents having a more rapid onset of therapeutic effects than the other.
74. In certain embodiments a method for treating a central nervous system disorder in a human comprising administering an effective amount of a pharmaceutical composition wherein the pharmaceutical composition comprises:
   (1) immediate release granules comprising a dopamine releasing agent and one or more pharmaceutically acceptable excipients;
   (2) delayed release granules comprising an entactogen and one or more pharmaceutically acceptable excipients; and
   (3) one or more additional pharmaceutically acceptable excipients;
wherein:
   the pharmaceutical composition provides a delay between the Tₘₐₓ of the dopamine releasing agent and the Tₘₐₓ of the entactogen of at least about 15 minutes
   the entactogenic compound has a serotonin release EC₅₀ of less than 10 μM; and
   the dopamine releasing agent has a dopamine release EC₅₀ of less than 10 μM.
75. In certain embodiments pharmaceutical composition is provided comprising:
   (1) immediate release granules comprising a dopamine releasing agent and one or more pharmaceutically acceptable excipients;
   (2) delayed release granules comprising an entactogen and one or more pharmaceutically acceptable excipients; and
   (3) one or more additional pharmaceutically acceptable excipients;
wherein:
   the pharmaceutical composition provides a delay between the Tₘₐₓ of the dopamine releasing agent and the Tₘₐₓ of the entactogen of at least about 15 minutes;
   the entactogenic compound has a serotonin release EC₅₀ of less than 10 μM; and
   the dopamine releasing agent has a dopamine release EC₅₀ of less than 10 μM.
76. The pharmaceutical composition of embodiment 75 for treating a central nervous system disorder in a human.

### Pharmaceutical Compositions and Salts

Pharmaceutical compositions typically comprise a pharmaceutically acceptable carrier, diluent, or excipient, and at least one active ingredient. “Pharmaceutically acceptable” as used in connection with an excipient, carrier, or diluent means an excipient, carrier, or diluent that is useful in preparing a pharmaceutical composition that is generally safe, non-toxic, and neither biologically nor otherwise undesirable for veterinary use and/or human pharmaceutical use. These compositions can be administered by a variety of routes including systemic, topical, parenteral, oral, mucosal (for example, buccal, sublingual), rectal, transdermal, subcutaneous, intravenous, intramuscular, inhaled, and intranasal. Such compositions are prepared in a manner well known in the pharmaceutical art and comprise at least one active compound. (See, for example, Remington, 2005, Remington: The science and practice of pharmacy, 21st ed., Lippincott Williams & Wilkins.)

The pharmaceutical composition may be formulated as any pharmaceutically useful form, for example, a solid dosage form, a liquid, an aerosol, a cream, a gel, a pill, an injection or infusion solution, a capsule, a tablet, a syrup, a transdermal patch, a subcutaneous patch, a dry powder, an inhalation formulation, a suppository, a buccal or sublingual formulation, a parenteral formulation, an ophthalmic solution, or in a medical device. Some dosage forms, such as tablets and capsules, are subdivided into suitably sized unit doses containing appropriate quantities of the active components, for example, an effective amount to achieve the desired purpose.

A “pharmaceutically acceptable composition” thus refers to at least one compound (which may be a mixture of enantiomers or diastereomers, as fully described herein) of the invention and a pharmaceutically acceptable vehicle, excipient, diluent or other carrier in an effective amount to treat a host, typically a human, who may be a patient.

In certain nonlimiting embodiments the pharmaceutical composition is a dosage form that contains from about 0.1 mg to about 1500 mg, from about 10 mg to about 1000 mg, from about 100 mg to about 800 mg, or from about 200 mg to about 600 mg of the active compound and optionally from about 0.1 mg to about 1500 mg, from about 10 mg to about 1000 mg, from about 100 mg to about 800 mg, or from about 200 mg to about 600 mg of an additional active agent in a unit dosage form. Examples are dosage forms with at least 0.1, 1, 5, 10, 20, 25, 40, 50, 100, 125, 150, 200, 250, 300, 400, 500, 600, 700, or 750 mg of active compound, or its salt or salt mixture.

The pharmaceutical compositions described herein can be formulated into any suitable dosage form, including aqueous oral dispersions, aqueous oral suspensions, solid dosage forms including oral solid dosage forms, aerosols, controlled release formulations, fast melt formulations, effervescent formulations, self-emulsifying dispersions, solid solutions, liposomal dispersions, lyophilized formulations, tablets, capsules, pills, powders, delayed-release formulations, immediate-release formulations, modified release formulations, extended-release formulations, pulsatile release formulations, multi particulate formulations, and mixed immediate release and controlled release formulations. Generally speaking, one will desire to administer an amount of the active agents of the present invention that is effective to achieve a plasma level commensurate with the concentrations found to be effective in vivo for a period of time effective to elicit a desired therapeutic effect without abuse liability.

In making the compositions employed in the present invention the active ingredient is usually mixed with an excipient, diluted by an excipient, or enclosed within such a carrier which can be in the form of a capsule, sachet, paper or other container. When the excipient serves as a diluent, it can be a solid, semi-solid, or liquid material, which acts as a vehicle, carrier, or medium for the active ingredient. Thus, the compositions can be in the form of tablets (including orally disintegrating, swallowable, sublingual, buccal, and chewable tablets), pills, powders, lozenges, troches, oral films, thin strips, sachets, cachets, elixirs, suspensions, emulsions, solutions, slurries, syrups, aerosols (as a solid or in a liquid medium), ointments containing for example up to 10% by weight of the active compound, soft and hard gelatin capsules, suppositories, dry powders for inhalation, liquid preparations for vaporization and inhalation, topical preparations, transdermal patches, sterile injectable solutions, and sterile packaged powders. Compositions may be formulated as immediate release, controlled release, sustained (extended) release or modified release formulations.

Other embodiments of the invention include multiple routes of administration, which may differ in different patients according to their preference, co-morbidities, side effect profile, and other factors (IV, PO, transdermal, etc.). Other embodiments of the invention include the presence of other substances with the active drugs, known to those skilled in the art, such as fillers, carriers, gels, skin patches, lozenges, or other modifications in the preparation to facilitate absorption through various routes (such as gastrointestinal, transdermal, etc.) and/or to extend the effect of the drugs, and/or to attain higher or more stable serum levels or to enhance the therapeutic effect of the active drugs in the combination.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, for example, about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxybenzoates; sweetening agents; and flavoring agents. The compositions of the invention can be formulated so as to provide quick, sustained or delayed release of the active ingredient after administration to the patient by employing procedures known in the art.

The compositions in certain non-limiting embodiments formulated in a unit dosage form, each dosage containing from about 0.05 to about 350 mg, more typically about 1.0 to about 180 mg, of the active ingredients. The term “unit dosage form” refers to physically discrete units suitable as unitary dosages for human subjects and other mammals, each unit containing a predetermined quantity of active material calculated to produce the desired therapeutic effect, in association with a suitable pharmaceutical carrier, diluent, or excipient.

For example, some dosages fall within the range of at least about 0.007 to about 4 mg/kg or less. In the treatment of adult humans, the range of at least about 0.1 to about 3 mg/kg or less, in single dose may be useful.

It will be understood that the amount of the compound actually administered will be determined by a physician, in light of the relevant circumstances, including the condition to be treated, the chosen route of administration, the actual compound or compounds administered, the age, weight, and response of the individual patient, and the severity of the patient's symptoms, and therefore the above dosage ranges are not intended to limit the scope of the invention in any way.

In some instances, dosage levels below the lower limit of the aforesaid range may be more than adequate, while in other cases still larger doses may be employed without causing any harmful side effects, provided for instance that such larger doses may be first divided into several smaller doses for administration.

Generally, the pharmaceutical compositions of the invention may be administered and dosed in accordance with good medical practice, taking into account the method and scheduling of administration, prior and concomitant medications and medical supplements, the clinical condition of the individual patient and the severity of the underlying disease, the patient's age, sex, body weight, and other such factors relevant to medical practitioners, and knowledge of the particular compound(s) used. Starting and maintenance dosage levels thus may differ from patient to patient, for individual patients across time, and for different pharmaceutical compositions, but shall be able to be determined with ordinary skill.

In other embodiments, a powder comprising the active agents of the present invention formulations described herein may be formulated to comprise one or more pharmaceutical excipients and flavors. Such a powder may be prepared, for example, by mixing the active agents of the present invention formulation and optional pharmaceutical excipients to form a bulk blend composition. Additional embodiments also comprise a suspending agent and/or a wetting agent. This bulk blend is uniformly subdivided into unit dosage packaging or multi-dosage packaging units. The term “uniform” means the homogeneity of the bulk blend is substantially maintained during the packaging process.

### Exemplary Oral Formulations

In certain embodiments, the dopamine releasing agent(s) and entactogen(s) may be formulated in a pharmaceutically acceptable oral dosage form. In some embodiments, the oral dosage form provides controlled release. Oral dosage forms may include but are not limited to, oral solid dosage forms and oral liquid dosage forms. Oral solid dosage forms may include but are not limited to, tablets, capsules, caplets, powders, pellets, multiparticulates, beads, spheres and/or any combinations thereof. These oral solid dosage forms may be formulated as immediate release, controlled release, sustained (extended) release or modified release formulations.

The oral solid dosage forms of the present invention may also contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof.

In some embodiments, the solid dosage forms of the present invention may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations of the present invention may be administered as a single capsule or in multiple capsule dosage form. In some embodiments, the pharmaceutical formulation is administered in two, or three, or four, capsules or tablets.

The pharmaceutical solid dosage forms described herein can comprise the active agent of the present invention compositions described herein and one or more pharmaceutically acceptable additives such as a compatible carrier, binder, complexing agent, ionic dispersion modulator, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the active agent of the present invention formulation. In one embodiment, some or all of the active agent of the present invention particles are coated. In another embodiment, some or all of the active agent of the present invention particles are microencapsulated. In yet another embodiment, some or all of the active agent of the present invention is amorphous material coated and/or microencapsulated with inert excipients. In still another embodiment, the active agent of the present invention particles not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose (e.g., Avicel®, Avicel® PH101, Avicel® PH102, Avicel® PH105, etc.), cellulose powder, dextrose, dextrates, dextrose, dextran, starches, pregelatinized starch, hydroxypropylmethylcellulose (HPMC), hydroxypropylmethylcellulose phthalate, hydroxypropylmethylcellulose acetate stearate (HPMCAS), sucrose, xylitol, lactitol, mannitol, sorbitol, sodium chloride, polyethylene glycol, and the like.

If needed, suitable disintegrants for use in the solid dosage forms described herein include, but are not limited to, natural starch such as corn starch or potato starch, a pregelatinized starch such as National 1551 or Amijel®, or a sodium starch glycolate such as Promogel® or Explotab®, a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, Ac-Di-Sol, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder-filled capsule formulation, they aid in plug formation that can be filled into soft or hard shell capsules and in tablet formulation, binders ensure that the tablet remains intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Agoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crosspovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like. In general, binder levels of 20-70% are used in powder-filled gelatin capsule formulations. Binder usage level in tablet formulations is a function of whether direct compression, wet granulation, roller compaction, or usage of other excipients such as fillers which itself can act as moderate binders are used. Formulators skilled in the art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumarate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Non-water-soluble diluents are compounds typically used in the formulation of pharmaceuticals, such as calcium phosphate, calcium sulfate, starches, modified starches and microcrystalline cellulose, and micro cellulose (e.g., having a density of about 0.45 g/cm3, e.g. Avicel, powdered cellulose), and talc.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like. Wetting agents include surfactants.

Suitable surfactants for use in the solid dosage forms described herein include, for example, docusate and its pharmaceutically acceptable salts, sodium lauryl sulfate, sorbitan monooleate, polyoxyethylene sorbitan monooleate, polysorbates, poloxamers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 18000, vinylpyrrolidone/vinyl acetate copolymer (S630), sodium alginate, gums, such as, e.g., gum tragacanth and gum acacia, guar gum, xanthans, including xanthan gum, sugars, cellulosic, such as, e.g., sodium carboxymethylcellulose, methylcellulose, sodium carboxymethylcellulose, hydroxypropylmethylcellulose, hydroxyethylcellulose, polysorbate-80, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), butyl hydroxyanisole (BHA), sodium ascorbate, Vitamin E TPGS, ascorbic acid, sorbic acid and tocopherol.

Immediate-release formulations may be prepared by combining super disintegrant such as Croscarmellose sodium and different grades of microcrystalline cellulose in different ratios. To aid disintegration, sodium starch glycolate will be added.

In cases where the two (or more) drugs included in the fixed-dose combinations of the present invention are incompatible, cross-contamination can be avoided, e.g. by incorporation of the drugs in different drug layers in the oral dosage form with the inclusion of a barrier layer(s) between the different drug layers, wherein the barrier layer(s) comprise one or more inert/non-functional materials.

The above-listed additives should be taken as merely examples and not limiting, of the types of additives that can be included in solid dosage forms of the present invention. The amounts of such additives can be readily determined by one skilled in the art, according to the particular properties desired.

Oral liquid dosage forms include, but are not limited to, solutions, emulsions, suspensions, and syrups. These oral liquid dosage forms may be formulated with any pharmaceutically acceptable excipient known to those of skill in the art for the preparation of liquid dosage forms. For example, water, glycerin, simple syrup, alcohol, and combinations thereof.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as but not limited to, an oil, water, an alcohol, and combinations of these pharmaceutically suitable surfactants, suspending agents, emulsifying agents, may be added for oral or parenteral administration. Suspensions may include oils. Such oils include, but are not limited to, peanut oil, sesame oil, cottonseed oil, corn oil, and olive oil. Suspension preparation may also contain esters of fatty acids such as ethyl oleate, isopropyl myristate, fatty acid glycerides, and acetylated fatty acid glycerides. Suspension formulations may include alcohols, such as, but not limited to, ethanol, isopropyl alcohol, hexadecyl alcohol, glycerol, and propylene glycol. Ethers, such as but not limited to, poly(ethylene glycol), petroleum hydrocarbons such as mineral oil and petrolatum; and water may also be used in suspension formulations.

In some embodiments, formulations are provided comprising the at least one dopamine releasing agent and at least one entactogen of the present invention particles described herein and at least one dispersing agent or suspending agent for oral administration to a subject. The formulation may be a powder and/or granules for suspension, and upon admixture with water, a substantially uniform suspension is obtained. As described herein, the aqueous dispersion can comprise amorphous and non-amorphous particles consisting of multiple effective particle sizes such that eg the dopamine releasing agent of the present invention comprise particles having a smaller effective particle size so that drug is absorbed more quickly and the entactogen particles have a larger effective particle size which are absorbed more slowly. In certain embodiments, the aqueous dispersion or suspension is an immediate-release formulation. In another embodiment, an aqueous dispersion comprising amorphous particles is formulated such that a portion of the dopamine releasing agent particles of the present invention are absorbed within, e.g., about 1.5 hours after administration and so that the entactogen particles are absorbed 1 to 3 hours after absorption of the dopamine releasing agent particles. In other embodiments, addition of a complexing agent to the aqueous dispersion results in a larger span of the dopamine releasing agent and entactogen particles to extend the drug absorption phase of the dopamine releasing agent such that 50-80% of the particles are absorbed in the first hour and about 90% are absorbed by about 4 hours and with the entactogen released 1 to 3 hours after administration of the composition. Dosage forms for oral administration can be aqueous suspensions selected from the group including, but not limited to, pharmaceutically acceptable aqueous oral dispersions, emulsions, solutions, and syrups. See, e.g., Singh et al., Encyclopedia of Pharmaceutical Technology, 2nd Ed., pp. 754-757 (2002). In addition to the active agents of the present invention particles, the liquid dosage forms may comprise additives, such as (a) disintegrating agents; (b) dispersing agents; (c) wetting agents; (d) at least one preservative, (e) viscosity enhancing agents, (f) at least one sweetening agent, and (g) at least one flavoring agent.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crosspovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropylcellulose ethers (e.g., HPC, HPC-SL, and HPC-L), hydroxypropylmethylcellulose and hydroxypropylmethylcellulose ethers (e.g. HPMC K100, HPMC K4M, HPMC K15M, and HPMC K100M), carboxymethylcellulose sodium, methylcellulose, hydroxyethylcellulose, hydroxypropylmethylcellulose phthalate, hydroxypropylmethylcellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; Tween®60 or 80; PEG; polyvinylpyrrolidone (PVP); hydroxypropyl cellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethylcellulose phthalate; hydroxypropylmethylcellulose acetate stearate; non-crystalline cellulose; magnesium aluminum silicate; triethanolamine; polyvinyl alcohol (PVA); 4-(1,1,3,3-tetramethyl butyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908®).

Wetting agents (including surfactants) suitable for the aqueous suspensions and dispersions described herein are known in the art and include, but are not limited to, acetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carpool 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphatidylcholine and the like.

Suitable preservatives for the aqueous suspensions or dispersions described herein include, for example, potassium sorbate, parabens (e.g., methylparaben and propylparaben) and their salts, benzoic acid and its salts, other esters of para hydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

In one embodiment, the aqueous liquid dispersion can comprise methylparaben and propylparaben in a concentration ranging from about 0.01% to about 0.3% methylparaben by weight to the weight of the aqueous dispersion and about 0.005% to about 0.03% propylparaben by weight to the total aqueous dispersion weight. In yet another embodiment, the aqueous liquid dispersion can comprise methylparaben from about 0.05 to about 0.1 weight % and propylparaben from about 0.01 to about 0.02 weight % of the aqueous dispersion.

Suitable viscosity enhancing agents for the aqueous suspensions or dispersions described herein include, but are not limited to, methyl cellulose, xanthan gum, carboxymethylcellulose, hydroxypropyl cellulose, hydroxypropylmethyl cellulose, Plasdone® S-630, carbomer, polyvinyl alcohol, alginates, acacia, chitosans and combinations thereof. The concentration of the viscosity-enhancing agent will depend upon the agent selected and the viscosity desired.

In addition to the additives listed above, the liquid active agents of the present invention formulations can also comprise inert diluents commonly used in the art, such as water or other solvents, solubilizing agents, emulsifiers, and/or sweeteners.

The active agents of the present invention formulations suitable for intramuscular, subcutaneous, or intravenous injection may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Additionally, the active agents of the present invention can be dissolved at concentrations of >1 mg/ml using water-soluble beta cyclodextrins (e.g. beta-sulfobutyl-cyclodextrin and 2-hydroxypropylbetacyclodextrin. Proper fluidity can be maintained, for example, by the use of a coating such as a lecithin, by the maintenance of the required particle size in the case of dispersions, and by the use of surfactants. The active agents of the present invention formulations suitable for subcutaneous injection may also contain additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, sorbic acid, and the like. It may also be desirable to include isotonic agents, such as sugars, sodium chloride, and the like. Prolonged drug absorption of the injectable pharmaceutical form can be brought about by the use of agents delaying absorption, such as aluminum monostearate and gelatin. The active agents of the present invention suspension formulations designed for extended-release via subcutaneous or intramuscular injection can avoid first-pass metabolism and lower dosages of the active agents of the present invention will be necessary to maintain plasma levels of about 50 ng/ml. In such formulations, the particle size of the active agents of the present invention particles and the range of the particle sizes of the active agents of the present invention particles can be used to control the release of the drug by controlling the rate of dissolution in fat or muscle.

In still other embodiments, effervescent powders containing at least dopamine releasing agent and at least one entactogen may be prepared. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the present invention are added to water, the acids and the base react to liberate carbon dioxide gas, thereby causing “effervescence.” Examples of effervescent salts include e.g: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In other embodiments, a powder comprising the active agents of the present invention formulations described herein may be formulated to comprise one or more pharmaceutical excipients and flavors. Such a powder may be prepared, for example, by mixing the active agents of the present invention formulation and optional pharmaceutical excipients to form a bulk blend composition. Additional embodiments also comprise a suspending agent and/or a wetting agent. This bulk blend is uniformly subdivided into unit dosage packaging or multi-dosage packaging units. The term “uniform” means the homogeneity of the bulk blend is substantially maintained during the packaging process

In certain embodiments of the present invention, pharmaceutical compositions containing a dopamine releasing agent and a entactogen may be formulated into a dosage form suitable for parenteral use. For example, the dosage form may be a lyophilized powder, a solution, suspension (e.g., depot suspension).

In other embodiments, pharmaceutical compositions containing one or more dopamine releasing agents and one or more entactogenic agents may be formulated into a topical dosage form such as, but not limited to, a patch, a gel, a paste, a cream, an emulsion, liniment, balm, lotion, and ointment.

Tablets of the invention described here can be prepared by methods well known in the art. Various methods for the preparation of the immediate release, modified release, controlled release, and extended-release dosage forms (e.g., as matrix tablets, tablets having one or more modified, controlled, or extended-release layers, etc.) and the vehicles therein are well known in the art. Generally recognized compendium of methods include: Remington: The Science and Practice of Pharmacy, Alfonso R. Gennaro, Editor, 20th Edition, Lippincott Williams & Wilkins, Philadelphia, PA; Sheth et al. (1980) Compressed tablets, in Pharmaceutical dosage forms, Vol 1, edited by Lieberman and Lachtman, Dekker, NY.

In certain embodiments, solid dosage forms, e.g., tablets, effervescent tablets, and capsules, are prepared by mixing the active agents of the present invention particles with one or more pharmaceutical excipients to form a bulk blend composition. When referring to these bulk blend compositions as homogeneous, it is meant that the active agents of the present invention particles are dispersed evenly throughout the composition so that the composition may be readily subdivided into equally effective unit dosage forms, such as tablets, pills, and capsules. The individual unit dosages may also comprise film coatings, which disintegrate upon oral ingestion or upon contact with diluents. These the active agents of the present invention formulations can be manufactured by conventional pharmaceutical techniques.

Conventional pharmaceutical techniques for preparation of solid dosage forms include, e.g., one or a combination of methods: (1) dry mixing, (2) direct compression, (3) milling, (4) dry or non-aqueous granulation, (5) wet granulation, or (6) fusion. See, e.g., Lachman et al., Theory and Practice of Industrial Pharmacy (1986). Other methods include, e.g., spray drying, pan coating, melt granulation, granulation, fluidized bed spray drying or coating (e.g., Wurster coating), tangential coating, top spraying, tableting, extruding and the like.

Compressed tablets are solid dosage forms prepared by compacting the bulk blend the active agents of the present invention formulations described above. In various embodiments, compressed tablets which are designed to dissolve in the mouth will comprise one or more flavoring agents. In other embodiments, the compressed tablets will comprise a film surrounding the final compressed tablet. In some embodiments, the film coating can provide a delayed release of the active agents of the present invention formulation, in particular, delayed release of the entactogen. In other embodiments, the film coating aids in patient compliance (e.g., Opadry® coatings or sugar coating). Film coatings comprising Opadry® typically range from about 1% to about 3% of the tablet weight. Film coatings for delayed-release usually comprise 2-6% of a tablet weight or 7-15% of a spray-layered bead weight. In other embodiments, the compressed tablets comprise one or more excipients.

A capsule may be prepared, e.g., by placing the bulk blend the active agents of the present invention formulation, described above, inside of a capsule. In some embodiments, the active agents of the present invention formulations (non-aqueous suspensions and solutions) are placed in a soft gelatin capsule. In other embodiments, the active agents of the present invention formulations are placed in standard gelatin capsules or non-gelatin capsules such as capsules comprising HPMC. In other embodiments, the active agents of the present invention formulations are placed in a sprinkle capsule, wherein the capsule may be swallowed whole or the capsule may be opened and the contents sprinkled on food prior to eating. In some embodiments of the present invention, the therapeutic dose is split into multiple (e.g., two, three, or four) capsules. In some embodiments, the entire dose of the active agents of the present invention formulation is delivered in a capsule form.

In certain preferred embodiments, the formulations of the present invention are fixed-dose combinations of at least one dopamine releasing agent and at least one entactogen. Fixed-dose combination formulations may contain the following combinations in the form of single-layer monolithic tablet or multi-layered monolithic tablet or in the form of a core tablet-in-tablet or multi-layered multi-disk tablet or beads inside a capsule or tablets inside a capsule but not limited to: (a) therapeutically efficacious fixed-dose combinations of immediate-release formulations of dopamine releasing agents and entactogens; (b) therapeutically efficacious fixed-dose combinations of immediate release dopamine releasing agent and delayed and/or extended-release entactogen contained in a single dosage form.

The pharmaceutical compositions described herein can be formulated into any suitable dosage form, including but not limited to, aqueous oral dispersions, aqueous oral suspensions, solid dosage forms including oral solid dosage forms, aerosols, controlled release formulations, fast melt formulations, effervescent formulations, self-emulsifying dispersions, solid solutions, liposomal dispersions, lyophilized formulations, tablets, capsules, pills, powders, delayed-release formulations, immediate-release formulations, modified release formulations, extended-release formulations, pulsatile release formulations, multi particulate formulations, and mixed immediate release and controlled release formulations. In some embodiments, the active agents of the present invention formulations provide a therapeutically effective amount of the dopamine releasing agent(s) of the present invention over an interval of about 30 minutes to about 2 hours after administration. Generally speaking, one will desire to administer an amount of the active agents of the present invention that is effective to achieve a plasma level commensurate with the concentrations found to be effective in vivo for a period of time effective to elicit a desired therapeutic effect without abuse liability.

Depending on the desired release profile, the oral solid dosage forms of the present invention may contain a suitable amount of controlled-release agents, extended-release agents, and/or modified-release agents (e.g., delayed-release agents). The pharmaceutical solid oral dosage forms comprising the active agents of the present invention described herein can be further formulated to provide a modified or controlled release of the active agents of the present invention. In some embodiments, the solid dosage forms described herein can be formulated as a delayed release dosage form such as an enteric-coated delayed release oral dosage forms, i.e., as an oral dosage form of a pharmaceutical composition as described herein which utilizes an enteric coating to affect release in the small intestine of the gastrointestinal tract. The enteric-coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated. Enteric coatings may also be used to prepare other controlled release dosage forms including extended-release and pulsatile release dosage forms.

In other embodiments, the active agents of the formulations described herein are delivered using a pulsatile dosage form. Pulsatile dosage forms comprising the active agents of the present invention formulations described herein may be administered using a variety of formulations known in the art. For example, such formulations include, but are not limited to, those described in U.S. Pat. Nos. 5,011,692, 5,017,381, 5,229,135, and 5,840,329, each of which is specifically incorporated by reference. Other dosage forms suitable for use with the active agents of the present invention formulations are described in, for example, U.S. Pat. Nos. 4,871,549, 5,260,068, 5,260,069, 5,508,040, 5,567,441 and 5,837,284, all of which are specifically incorporated by reference. In one embodiment, the controlled release dosage form is pulsatile release solid oral dosage form comprising at least two groups of particles, each containing active agents of the present invention as described herein. The first group of particles provides a substantially immediate dose of the active agents of the present invention upon ingestion by a subject. The first group of particles can be either uncoated or comprise a coating and/or sealant. The second group of particles comprises coated particles, which may comprise from about 2% to about 75%, preferably from about 2.5% to about 70%, or from about 40% to about 70%, by weight of the total dose of the active agents of the present invention in said formulation, in admixture with one or more binders.

Coatings for providing a controlled, delayed, or extended-release may be applied to the at least one dopamine releasing agent and/or the at least one entactogen or to a core containing the at least one dopamine releasing agent and/or the at least one entactogen. The coating may comprise a pharmaceutically acceptable ingredient in an amount sufficient, e.g., to provide a delay of from e.g. about 0.5 hours to about 2 hours following ingestion before release of the entactogen. Suitable coatings include one or more differentially degradable coatings such as, by way of example only, pH-sensitive coatings (enteric coatings) such as acrylic resins (e.g., Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, and Eudragit® NE30D, Eudragit® NE 40D®) either alone or blended with cellulose derivatives, e.g., ethylcellulose, or non-enteric coatings having variable thickness to provide differential release of the active agents of the present invention formulation.

Many other types of controlled/delayed/extended-release systems known to those of ordinary skill in the art and are suitable for use with the active agents of the present invention formulations described herein. Examples of such delivery systems include, e.g., polymer-based systems, such as polylactic and polyglycolic acid, polyanhydrides and polycaprolactone, cellulose derivatives (e.g., ethylcellulose), porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2nd Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983, each of which is specifically incorporated by reference. In certain embodiments, the controlled release systems may comprise the controlled/delayed/extended-release material incorporated with the drug(s) into a matrix, whereas in other formulations, the controlled release material may be applied to a core containing the drug(s). In certain embodiments, one drug may be incorporated into the core while the other drug is incorporated into the coating. In some embodiments, materials include shellac, acrylic polymers, cellulosic derivatives, polyvinyl acetate phthalate, and mixtures thereof. In other embodiments, materials include Eudragit® series E, L, RL, RS, NE, L, L300, S, 100-55, cellulose acetate phthalate, Aquateric, cellulose acetate trimellitate, ethyl cellulose, hydroxypropylmethylcellulose phthalate, hydroxypropylmethylcellulose acetate succinate, polyvinyl acetate phthalate, and Cotteric. The controlled/delayed/extended-release systems may utilize a hydrophilic polymer, including but not limited to a water-swellable polymer (e.g., a natural or synthetic gum). The hydrophilic polymer may be any pharmaceutically acceptable polymer which swells and expands in the presence of water to slowly release the active agents of the present invention. These polymers include polyethylene oxide, methylcellulose, hydroxypropyl cellulose, hydroxypropylmethylcellulose, and the like.

The performance of acrylic polymers (primarily their solubility in biological fluids) can vary based on the degree and type of substitution. Examples of suitable acrylic polymers which may be used in matrix formulations or coatings include methacrylic acid copolymers and ammonia methacrylate copolymers. The Eudragit series E, L, S, RL, RS and NE (Rohm Pharma) are available as solubilized in an organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in the stomach and dissolve in the intestine; Opadry Enteric is also insoluble in the stomach and dissolves in the intestine.

Examples of suitable cellulose derivatives for use in matrix formulations or coatings include ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous-based system and is a spray-dried CAP psuedolatex with particles<1 μm. Other components in Aquateric can include pluronic, Tweens, and acetylated monoglycerides. Other suitable cellulose derivatives include cellulose acetate trimellitate (Eastman); methylcellulose (Pharmacoat, Methocel); hydroxypropylmethylcellulose phthalate (HPMCP); hydroxypropylmethylcellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-555, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules or as fine powders for aqueous dispersions. Other suitable cellulose derivatives include hydroxypropylmethylcellulose.

In some embodiments, the coating may contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate, and triacetin. Conventional coating techniques such as spray or pan coating are employed to apply coatings. The coating thickness must be sufficient to ensure that the oral dosage form remains intact until the desired site of topical delivery in the intestinal tract is reached.

Extended-release multi-layered matrix tablets may be prepared by using fixed-dose combinations of at least one dopamine releasing agent together with at least 1 entactogen. Such formulations may comprise one or more of the drugs within a hydrophilic or hydrophobic polymer matrix. For example, a hydrophilic polymer may comprise guar gum, hydroxypropylmethylcellulose, and xanthan gum as matrix formers. Lubricated formulations may be compressed by a wet granulation method.

Multilayer tablet delivery (e.g., such as that used in the GeoMatrix™ technology) comprises a hydrophilic matrix core containing the active ingredient and one or two impermeable or semi-permeable polymeric coatings. This technology uses films or compressed polymeric barrier coatings on one or both sides of the core. The presence of polymeric coatings (e.g., such as that used in the GeoMatrix™ technology) modifies the hydration/swelling rates of the core and reduces the surface area available for drug release. These partial coatings provide modulation of the drug dissolution profile: they reduce the release rate from the device and shift the typical time-dependent release rate towards constant release. This technology enables customized levels of controlled release of specific drugs and/or simultaneous release of two different drugs at different rates that can be achieved from a single tablet. The combination of layers, each with different rates of swelling, gelling and erosion, is used for the rate of drug release in the body. Exposure of the multilayer tablet as a result of the partial coating may affect the release and erosion rates, therefore, transformation of a multilayered tablet with exposure on all sides to the gastrointestinal fluids upon detachment of the barrier layer will be considered.

Multi-layered tablets containing combinations of immediate release and modified/extended release of two different drugs or dual release rate of the same drug in a single dosage form may be prepared by using hydrophilic and hydrophobic polymer matrices.

Dual release repeat action multi-layered tablets may be prepared with an outer compression layer with an initial dose of rapidly disintegrating matrix in the stomach and a core inner layer tablet formulated with components that are insoluble in the gastric media but release efficiently in the intestinal environment.

In one embodiment, the dosage form is a solid oral dosage form which is an immediate release dosage form whereby >80% of the active agents of the present invention particles hours after administration. In other embodiments, the invention provides an (e.g., solid oral) dosage form that is a controlled release or pulsatile release dosage form. In such instances, the release may be, e.g., 30 to 60% of the active agents of the present invention particles by weight are released from the dosage form within about 2 hours after administration and about 90% by weight of the active agents of the present invention released from the dosage form, e.g., within about 7 hours after administration. In yet other embodiments, the dosage form includes at least one active agent in an immediate-release form and at least one active agent in the delayed-release form or sustained-release form. In yet other embodiments, the dosage form includes at least two active agents that are released at different rates as determined by in-vitro dissolution testing or via oral administration.

The various release dosage formulations discussed above, and others known to those skilled in the art can be characterized by their disintegration profile. A profile is characterized by the test conditions selected. Thus, the disintegration profile can be generated at a pre-selected apparatus type, shaft speed, temperature, volume, and pH of the dispersion media. Several disintegration profiles can be obtained. For example, a first disintegration profile can be measured at a pH level approximating that of the stomach (about pH 1.2); a second disintegration profile can be measured at a pH level approximating that of one point in the intestine or several pH levels approximating multiple points in the intestine (about 6.0 to about 7.5, more specifically, about 6.5 to 7.0). Another disintegration profile can be measured using distilled water. The release of formulations may also be characterized by their pharmacokinetic parameters, for example, Cmax, Tₘₐₓ, and AUC (0-τ).

In certain embodiments, the controlled, delayed or extended-release of one or more of the drugs of the fixed-dose combinations of the invention may be in the form of a capsule having a shell comprising the material of the rate-limiting membrane, including any of the coating materials previously discussed, and filled with the active agents of the present invention particles. A particular advantage of this configuration is that the capsule may be prepared independently of the active agent of the present invention particles; thus process conditions that would adversely affect the drug can be used to prepare the capsule. Alternatively, the formulation may comprise a capsule having a shell made of a porous or a pH-sensitive polymer made by a thermal forming process. Another alternative is a capsule shell in the form of an asymmetric membrane, i.e., a membrane that has a thin skin on one surface and most of whose thickness is constituted of a highly permeable porous material. The asymmetric membrane capsules may be prepared by a solvent exchange phase inversion, wherein a solution of polymer, coated on a capsule-shaped mold, is induced to phase-separate by exchanging the solvent with a miscible non-solvent. In another embodiment, spray layered active agents of the present invention particles are filled in a capsule. An exemplary process for manufacturing the spray layered the active agents of the present invention is the fluidized bed spraying process. The active agents of the present invention suspensions or the active agents of the present invention complex suspensions described above may be sprayed onto sugar or microcrystalline cellulose (MCC) beads (20-35 mesh) with Wurster column insert at an inlet temperature of 50° C. to 60° C. and air temp of 30° C. to 50° C. A 15 to 20 wt % total solids content suspension containing 45 to 80 wt % the active agents of the present invention, 10 to 25 wt % hydroxymethylpropylcellulose, 0.25 to 2 wt % of SLS, 10 to 18 wt % of sucrose, 0.01 to 0.3 wt % simethicone emulsion (30% emulsion) and 0.3 to 10% NaCl, based on the total weight of the solid content of the suspension, are sprayed (bottom spray) onto the beads through 1.2 mm nozzles at 10 mL/min and 1.5 bar of pressure until a layering of 400 to 700% wt % is achieved as compared to initial beads weight. The resulting spray layered the active agents of the present invention particles or the active agents of the present invention complex particles comprise about 30 to 70 wt % of the active agents of the present invention based on the total weight of the particles. In one embodiment the capsule is a size 0 soft gelatin capsule. In one embodiment, the capsule is a swelling plug device. In another embodiment, the swelling plug device is further coated with cellulose acetate phthalate or copolymers of methacrylic acid and methylmethacrylate. In some embodiments, the capsule includes at least 100 mg (or at least 300 mg or at least 400 mg) of the active agents of the present invention and has a total weight of less than 800 mg (or less than 700 mg). The capsule may contain a plurality of the active agents of the present invention-containing beads, for example, spray layered beads. In some embodiments, the beads are 12-25% the active agents of the present invention by weight. In some embodiments, some or all of the active agents of the present invention containing beads are coated with a coating comprising 6 to 15% (or 8 to 12%) of the total bead weight. Optimization work typically involves lower loading levels and the beads constitute 30 to 60% of the finished bead weight. The capsule may contain a granulated composition, wherein the granulated composition comprises the active agents of the present invention.

The capsule may provide pulsatile release the active agents of the present invention oral dosage form. Such formulations may comprise: (a) a first dosage unit comprising at least one dopamine releasing agent of the present invention that is released substantially immediately following oral administration of the dosage form to a patient; (b) a second dosage unit comprising at least one entactogen of the present invention dose that is released approximately 0.5 to 4 hours following administration of the dosage form to a patient. For pulsatile release capsules containing beads, the beads can be coated with a coating comprising 6 to 15% (or 8 to 12%) of the total bead weight. In some embodiments, the coating is a coating that is insoluble at pH 1 to 2 and soluble at pH greater than 5.5. In certain embodiments, the formulation may comprise a pulsatile release capsule comprising at least one dopamine releasing agent and at least one entactogen. This pulsatile release capsule may contain a plurality of beads in which the dopamine releasing agent beads are immediate-release beads and the entactogen beads are formulated, for example with the use of a coating, for modified release, typically from about 0.5 to 4 hours after administration. In other embodiments, the pulsatile release capsule contains a plurality of entactogen beads formulated for modified release and the at least one dopamine releasing agent of the present invention are, for example, spray granulated for immediate release.

In some embodiments, the release of the active agents of the present invention uses particles, and in particular preferably the entactogen particles, can be modified with a modified release coating, such as an enteric coating using cellulose acetate phthalate or a sustained release coating comprising copolymers of methacrylic acid and methylmethacrylate. In one embodiment, the enteric coating may be present in an amount of about 0.5 to about 15 wt %, more specifically, about 8 to about 12 wt %, based on the weight of, e.g., the spray layered particles. In one embodiment, the spray layered particles coated with the delayed and/or sustained release coatings can be filled in a modified release capsule in which both enteric-coated entactogen particles and immediate release dopamine releasing agent particles of the present invention beads are filled into a soft gelatin capsule. Additional suitable excipients may also be filled with the coated particles in the capsule. The uncoated particles release the dopamine releasing agent(s) of the present invention immediately upon administration while the coated particles do not release the entactogen of the present invention until these particles reach the intestine. By controlling the ratios of the coated and uncoated particles, desirable pulsatile release profiles also may be obtained. In some embodiments, the ratios between the uncoated and the coated particles are e.g., 20/80, or 30/70, or 40/60, or 50/50, w/w to obtain desirable release.

In certain embodiments, the dopamine releasing agent and/or entactogen drugs contained in a fixed-dose combination of the present invention may be in the form of beads contained within a capsule. In certain embodiments, some beads may release one or both drugs immediately, while other beads would release one or both drugs over an extended period of time or after a delay (delayed-release).

In certain embodiments, spray layered active agents of the present invention can be compressed into tablets with commonly used pharmaceutical excipients. Any appropriate apparatus for forming the coating can be used to make the enteric coated tablets, e.g., fluidized bed coating using a Wurster column, powder layering in coating pans or rotary coaters; dry coating by double compression technique; tablet coating by film coating technique, and the like. See, e.g., U.S. Pat. No. 5,322,655; Remington's Pharmaceutical Sciences Handbook: Chapter 90 “Coating of Pharmaceutical Dosage Forms”, 1990. In certain embodiments, the spray layered active agents of the present invention described above and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the active agents of the present invention formulation into the gastrointestinal fluid. In other embodiments, the spray layered active agents of the present invention particles or spray layered active agents complex particles with enteric coatings described above and one or more excipients are dry blended and compressed into a mass, such as a tablet. In preferred embodiments, the at least one dopamine releasing agent is spray layered for immediate release and the at least one entactogen is enteric-coated particles in the tablet to substantially avoid the release of the at least one entactogen of the present invention, for example, less than 15 wt %, in the stomach but releases substantially all the entactogen of the present invention (enterically or sustained-release coated), for example, greater than 80 wt %, in the intestine.

In certain embodiments, a pulsatile release of the active agent of the present invention formulation comprises a first dosage unit comprising a formulation made from the active agent of the present invention containing granules made from a spray drying or spray granulated procedure or a formulation made from the active agent of the present invention complex containing granules made from a spray drying or spray granulated procedure without enteric or sustained-release coatings and a second dosage unit comprising spray layered the active agent of the present invention particles or spray layered the active agent of the present invention complex particles with enteric or sustained-release coatings. In preferred embodiments, the at least one dopamine releasing agent is without enteric or sustained-release coatings and the at least one entactogen is spray layered with enteric or sustained-release coatings. In one embodiment, the dopamine releasing agent and entactogen are wet or dry blended and compressed into a mass to make a pulsatile release tablet.

In certain embodiments, binding, lubricating and disintegrating agents are blended (wet or dry) to the spray layered active agent of the present invention to make a compressible blend. The dopamine releasing agent dosage units and the entactogen dosage units are compressed separately and then compressed together to form a bilayer tablet. In yet another embodiment, the dopamine releasing agent dosage unit is in the form of an overcoat and completely covers the second dosage unit.

In certain embodiments, ingredients (including or not including the active agent) of the invention are wet granulated. The individual steps in the wet granulation process of tablet preparation include milling and sieving of the ingredients, dry powder mixing, wet massing, granulation, drying, and final grinding. In various embodiments, the active agents of the present invention composition are added to the other excipients of the pharmaceutical formulation after they have been wet granulated. Alternatively, the ingredients may be subjected to dry granulation, e.g., via compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). No wet binder or moisture is involved in any of the steps. In some embodiments, the active agents of the present invention formulation are dry granulated with other excipients in the pharmaceutical formulation. In other embodiments, the active agents of the present invention formulation are added to other excipients of the pharmaceutical formulation after they have been dry granulated.

In other embodiments, the formulation of the present invention formulations described herein is a solid dispersion. Methods of producing such solid dispersions are known in the art and include, but are not limited to, for example, U.S. Pat. Nos. 4,343,789, 5,340,591, 5,456,923, 5,700,485, 5,723,269, and U.S. Pub. Appl. 2004/0013734, each of which is specifically incorporated by reference. In some embodiments, the solid dispersions of the invention comprise both amorphous and non-amorphous active agents of the present invention and can have enhanced bioavailability as compared to conventional active agents of the present invention formulations. In still other embodiments, the active agents of the present invention formulations described herein are solid solutions. Solid solutions incorporate a substance together with the active agent and other excipients such that heating the mixture results in the dissolution of the drug and the resulting composition is then cooled to provide a solid blend that can be further formulated or directly added to a capsule or compressed into a tablet.

The pharmaceutical agents which make up the combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of the target molecule concentration may also determine the optimal dose interval. For example, the at least one dopamine releasing agent may be administered while the at least one entactogen is being administered (concurrent administration) or may be administered before or after the drug from entactogen is administered (sequential administration).

In preferred embodiments, the release profile of the entactogen is delayed release, with the lag time (time until release initiation) depending on the pharmacology of the dopamine releasing agent and selected to allow a continuous period of useful therapeutic effects between 4 and 8 hours in duration.

Techniques to achieve these types of controlled release that are known to those skilled in the art include, but are not limited to: (1) use of osmotic or diffusion systems where an active substance is released over time through one or more small openings in an outer membrane; (2) slow dissolving polymers, with dissolution dependent on time, pH, or both, to coat tablets or to coat beads, pellets, minitablets, microcapsules, drug particles or similar which are packaged in capsules or tablets; and (3) chemical modification of active substances to form prodrugs that are transformed into active ingredients inside the body. In the case of osmotic or diffusion systems, material within the membrane may be designed to swell and force an active substance through the membrane. In the case of slow dissolving coatings, different coatings or thicknesses of coatings can enable the drug to be released in a staggered effect, as in repeat action release.

One exemplative approach for sustained release of the dopamine releasing agent is coating with Eudragit® RL and RS which have pH-independent solubility. Customized release profile is achieved by combination of RL and RS grades in different ratios.

One exemplative approach for delayed release of the entactogen uses colon-targeted drug delivery achieved by embedding the drug in polymer matrices to trap it and release it in the colon. These matrices can be pH-sensitive or biodegradable. In one exemplative formulation, Eudragit® S100, a methacrylic-acid based polymer that dissolves above pH 7.0, is used as an outer coating over the entactogen to ensure dissolution begins in the ileocecal region at the terminal ileum, while a second inner coating, such as Eudragit® L100-55 (which dissolves above pH 5.5), is used to ensure entactogen release begins in the proximal colon, which is typically reached 3.35 to 6 hours after ingestion.

Examples of specific agents and ranges of doses are given in the Table below.

TABLE 8
illustrative dopamine releasing agents and entactogens

| Earlier Agent | Later Agent |
|---|---|
| 2-40 mg amphetamine | 10-200 mg RS-MDMA |
| 2-40 mg methamphetamine | 10-200 mg RS-MDMA |
| 25-500 mg BK-MDMA | 10-200 mg RS-MDMA |
| 10-180 mg S-MDMA | 10-180 mg R-MDMA |
| 5-120 mg enantiomerically | 5-120 mg enantiomerically |
| enriched 6-MBPB (more R) | enriched 6-MBPB (more S) |
| 5-120 mg enantiomerically | 5-120 mg enantiomerically |
| enriched 5-MBPB (more R) | enriched 5-MBPB (more S) |
| 5-120 mg S-5-MAPB | 5-120 mg R-5-MAPB |
| 2-40 mg amphetamine | 5-120 mg RS-5-MAPB |
| 2-40 mg methamphetamine | 5-120 mg RS-5-MAPB |
| 5-400 mg 4-MMC | 10-200 mg RS-MDMA |
| 5-400 mg 4-MMC | 10-200 mg RS-MDMA |
| 5-400 mg R-4-MMC | 5-400 mg S-4-MMC |
| 10-150 mg S-MDA | 10-150 mg R-MDA |

In certain embodiments the kinetic lag utilized in the present invention is accomplished by administering one compound before the other. In other embodiments the two compounds are administered at about the same time but by different routes of administration (for example the compound with initial activity may be administered intravenously while the delayed compound is administered orally). In other embodiments the two compounds are administered at the same time within a pharmaceutical composition that delays the therapeutic effects of one of the two compounds. Alternatively, the two compounds may be administered as salts wherein one salt delays the therapeutic effect and the other has no effect or speeds the therapeutic effect.

In some aspects one enantiomer of the entactogenic compound described herein is an entactogen for use in combination with the dopamine releasing agent and the other enantiomer is the dopamine releasing agent. In this aspect there is a kinetic lag between the effect of the two compounds which is accomplished by the techniques described herein.

### Additional Pharmaceutical Compositions

The tryptamine compounds and compositions described herein can be administered in an effective amount as the neat chemical but are more typically administered as a pharmaceutical composition for a host, typically a human, in need of such treatment in an effective amount for any of the disorders described herein. The pharmaceutical composition typically comprises a pharmaceutically acceptable carrier, diluent, or excipient, and at least one compound, pure enantiomer, or enantiomerically enriched mixture of the present invention. The compounds or compositions disclosed herein may be administered orally, topically, systemically, parenterally, by inhalation, insufflation, or spray, mucosally (for example, buccal, sublingual), sublingually, transdermally, rectally, intravenous, intra-aortal, intracranial, subdermal, intraperitoneal, intramuscularly, inhaled, intranasal, subcutaneous, transnasal, or by other means, in dosage unit formulations containing conventional pharmaceutically acceptable carriers. Such compositions are prepared in a manner well known in the pharmaceutical art and comprise at least one active compound. (See, for example, Remington, 2005, Remington: The science and practice of pharmacy, 21st ed., Lippincott Williams & Wilkins.)

The pharmaceutical composition may be formulated as any pharmaceutically useful form, for example, as an aerosol, a cream, a gel, a pill, an injection or infusion solution, a capsule, a tablet, a syrup, a transdermal patch, a subcutaneous patch, a dry powder, an inhalation formulation, a suppository, a buccal or sublingual formulation, a parenteral formulation, an ophthalmic solution, or in a medical device. Some dosage forms, such as tablets and capsules, are subdivided into suitably sized unit doses containing appropriate quantities of the active components, for example, an effective amount to achieve the desired purpose.

A “pharmaceutically acceptable composition” thus refers to at least one compound (which may be a mixture of enantiomers or diastereomers, as fully described herein) of the invention and a pharmaceutically acceptable vehicle, excipient, diluent or other carrier in an effective amount to treat a host, typically a human, who may be a patient.

In certain nonlimiting embodiments the pharmaceutical composition is a dosage form that contains from about 0.1 mg to about 1500 mg, from about 10 mg to about 1000 mg, from about 100 mg to about 800 mg, or from about 200 mg to about 600 mg of the active compound and optionally from about 0.1 mg to about 1500 mg, from about 10 mg to about 1000 mg, from about 100 mg to about 800 mg, or from about 200 mg to about 600 mg of an additional active agent in a unit dosage form. Examples are dosage forms with at least 0.1, 1, 5, 10, 20, 25, 40, 50, 100, 125, 150, 200, 250, 300, 400, 500, 600, 700, or 750 mg of active compound, or its salt or salt mixture.

The pharmaceutical compositions described herein can be formulated into any suitable dosage form, including tablets, capsules, gelcaps, aqueous oral dispersions, aqueous oral suspensions, solid dosage forms including oral solid dosage forms, aerosols, controlled release formulations, fast melt formulations, effervescent formulations, self-emulsifying dispersions, solid solutions, liposomal dispersions, lyophilized formulations, pills, powders, delayed-release formulations, immediate-release formulations, modified release formulations, extended-release formulations, pulsatile release formulations, multi particulate formulations, and mixed immediate release and controlled release formulations. Generally speaking, the composition should be administered in an effective amount to administer an amount of the active agents of the present invention achieves a plasma level commensurate with the concentrations found to be effective in vivo for a period of time effective to elicit a desired therapeutic effect without abuse liability.

In making the compositions employed in the present invention the active ingredient is usually mixed with an excipient, diluted by an excipient, or enclosed within such a carrier which can be in the form of a capsule, sachet, paper or other container. When the excipient serves as a diluent, it can be a solid, semi-solid, or liquid material, which acts as a vehicle, carrier, or medium for the active ingredient. Thus, the compositions can be in the form of tablets (including orally disintegrating, swallowable, sublingual, buccal, and chewable tablets), pills, powders, lozenges, troches, oral films, thin strips, sachets, cachets, elixirs, suspensions, emulsions, solutions, slurries, syrups, aerosols (as a solid or in a liquid medium), ointments containing for example up to 10% by weight of the active compound, soft and hard gelatin capsules, suppositories, dry powders for inhalation, liquid preparations for vaporization and inhalation, topical preparations, transdermal patches, sterile injectable solutions, and sterile packaged powders. Compositions may be formulated as immediate release, controlled release, sustained (extended) release or modified release formulations.

The compositions of the present invention can be administered by multiple routes, which may differ in different patients according to their preference, co-morbidities, side effect profile, and other factors (IV, PO, transdermal, etc.). In one embodiment, the pharmaceutical composition includes the presence of other substances with the active drugs, known to those skilled in the art, such as fillers, carriers, gels, skin patches, lozenges, or other modifications in the preparation to facilitate absorption through various routes (such as, but not limited to, gastrointestinal, transdermal, etc.) and/or to extend the effect of the drugs, and/or to attain higher or more stable serum levels or to enhance the therapeutic effect of the active drugs in the combination.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, for example, about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include, but are not limited to, lubricating agents such as talc, magnesium stearate, and mineral oil; wetting agents; emulsifying and suspending agents; preserving agents such as methyl- and propylhydroxybenzoates; sweetening agents; and flavoring agents. The compositions of the invention can be formulated so as to provide quick, sustained or delayed release of the active ingredient after administration to the patient by employing procedures known in the art.

The compositions are in certain embodiments formulated in a unit dosage form, each dosage containing from at least about 0.05 to about 350 mg or less, more typically at least about 0.1 to about 280 mg or less, of the active ingredients. The term “unit dosage form” refers to physically discrete units suitable as unitary dosages for human subjects and other mammals, each unit containing a predetermined quantity of active material calculated to produce the desired therapeutic effect, in association with a suitable pharmaceutical carrier, diluent, or excipient.

The active compounds are effective over a wide dosage range. For example, as-needed dosages normally fall within the range of at least about 0.0007 to about 5 mg/kg or less. In the treatment of adult humans, the range of at least about 0.001 to about 4 mg/kg or less, in single dose may be useful.

It will be understood that the amount of the compound actually administered will be determined by a physician, in light of the relevant circumstances, including the condition to be treated, the chosen route of administration, the actual compound or compounds administered, the age, weight, and response of the individual patient, and the severity of the patient's symptoms, and therefore the above dosage ranges are not intended to limit the scope of the invention in any way.

In some instances, dosage levels below the lower limit of the aforesaid range may be more than adequate, while in other cases still larger doses may be employed without causing any harmful side effects, provided for instance that such larger doses may be first divided into several smaller doses for administration.

Generally, the pharmaceutical compositions of the invention may be administered and dosed in accordance with good medical practice, taking into account the method and scheduling of administration, prior and concomitant medications and medical supplements, the clinical condition of the individual patient and the severity of the underlying disease, the patient's age, sex, body weight, and other such factors relevant to medical practitioners, and knowledge of the particular compound(s) used. Starting and maintenance dosage levels thus may differ from patient to patient, for individual patients across time, and for different pharmaceutical compositions, but shall be able to be determined with ordinary skill.

In one embodiment, a powder comprising the active agents of the present invention described herein may be formulated to comprise one or more pharmaceutical excipients and flavors. Such a powder may be prepared, for example, by mixing the active agents of the present invention and optional pharmaceutical excipients to form a bulk blend composition. Additional embodiments also comprise a suspending agent and/or a wetting agent. This bulk blend is uniformly subdivided into unit dosage packaging or multi-dosage packaging units. The term “uniform” means the homogeneity of the bulk blend is substantially maintained during the packaging process.

### Oral Formulations

In certain embodiments, any selected compound(s) of the present invention is formulated in an effective amount in a pharmaceutically acceptable oral dosage form. In one embodiment, the compound(s) is a compound described herein or a pharmaceutically acceptable salt thereof. Oral dosage forms may include, but are not limited to, oral solid dosage forms and oral liquid dosage forms. Oral solid dosage forms may include but are not limited to, tablets, capsules, caplets, powders, pellets, multiparticulates, beads, spheres and/or any combinations thereof. The oral solid dosage forms may be formulated as immediate release, controlled release, sustained (extended) release or modified release formulations.

The oral solid dosage forms of the present invention may also contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof.

In some embodiments, the solid dosage forms of the present invention may be in the form of a tablet (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, for example, capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including a fast-melt tablet. Additionally, pharmaceutical formulations of the present invention may be administered as a single capsule or in multiple capsule dosage form. In some embodiments, the pharmaceutical formulation is administered in two, or three, or four, capsules or tablets.

The pharmaceutical solid dosage forms described herein can comprise the active agent of the present invention compositions described herein and one or more pharmaceutically acceptable additives such as a compatible carrier, binder, complexing agent, ionic dispersion modulator, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

Alternatively, the pharmaceutical solid dosage forms described herein can comprise the active agent or agents of the present invention (i.e., the “active agent(s)”; but for convenience herein, both “active agent” and “active agents” shall mean “active agent(s)” unless context clearly indicates that what is intended or would be suitable is only one agent or only two or more agents) and one or more pharmaceutically acceptable additives such as a compatible carrier, binder, complexing agent, ionic dispersion modulator, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, such as those described in Remington's Pharmaceutical Sciences, 20th Edition (2000), a film coating is provided around the active agent of the present invention formulation. In one embodiment, some or all of the active agent of the present invention particles are coated. In another embodiment, some or all of the active agent of the present invention particles are microencapsulated. In yet another embodiment, some or all of the active agent of the present invention is amorphous material coated and/or microencapsulated with inert excipients. In still another embodiment, the active agent of the present invention particles are not microencapsulated and are uncoated.

Suitable carriers for use in the solid dosage forms described herein include acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include lactose, calcium carbonate, calcium phosphate, dibasic calcium phosphate, calcium sulfate, microcrystalline cellulose (for example, Avicel®, Avicel® PH101, Avicel® PH102, Avicel® PH105, etc.), cellulose powder, dextrose, dextrates, dextrose, dextran, starches, pregelatinized starch, hydroxypropylmethylcellulose (HPMC), hydroxypropylmethylcellulose phthalate, hydroxypropylmethylcellulose acetate stearate (HPMCAS), sucrose, xylitol, lactitol, mannitol, sorbitol, sodium chloride, polyethylene glycol, and the like.

If needed, suitable disintegrants for use in the solid dosage forms described herein include natural starch such as corn starch or potato starch, a pregelatinized starch such as National 1551 or Amijel®, or a sodium starch glycolate such as Promogel® or Explotab®, a cellulose such as a wood product, microcrystalline cellulose, for example, Avicel®, Avicel® PH101, Avicel® PH102, Avicel® PH105, Elcema® P100, Emcocel®, Vivacel®, Ming Tia®, and Solka-Floc®, Ac-Di-Sol, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder-filled capsule formulation, they aid in plug formation that can be filled into soft- or hard-shell capsules and in tablet formulation, binders ensure that the tablet remains intact after compression and help assure blend uniformity prior to a compression or fill step. Materials suitable for use as binders in the solid dosage forms described herein include carboxymethylcellulose, methylcellulose (for example, Methocel®), hydroxypropylmethylcellulose (for example, Hypromellose USP Pharmacoat-603, hydroxypropylmethylcellulose acetate stearate (Agoate HS-LF and HS), hydroxyethylcellulose, hydroxypropylcellulose (for example, Klucel®), ethylcellulose (for example, Ethocel®), and microcrystalline cellulose (for example, Avicel®), microcrystalline dextrose, amylose, magnesium aluminum silicate, polysaccharide acids, bentonites, gelatin, polyvinylpyrrolidone/vinyl acetate copolymer, crosspovidone, povidone, starch, pregelatinized starch, tragacanth, dextrin, a sugar, such as sucrose (for example, Dipac®), glucose, dextrose, molasses, mannitol, sorbitol, xylitol (for example, Xylitab®), lactose, a natural or synthetic gum such as acacia, tragacanth, ghatti gum, mucilage of isapol husks, starch, polyvinylpyrrolidone (for example, Povidone® CL, Kollidon® CL, Polyplasdone® XL-10, and Povidone® K-12), larch arabogalactan, Veegum®, polyethylene glycol, waxes, sodium alginate, and the like. In general, binder levels of 20-70% are typically used in powder-filled gelatin capsule formulations. Binder usage level in tablet formulations is a function of whether direct compression, wet granulation, roller compaction, or usage of other excipients such as fillers which itself can act as moderate binders are used. Formulators skilled in the art can determine the binder level for the formulations, but binder usage level of up to 70% in tablet formulations is common.

Suitable lubricants or glidants for use in the solid dosage forms described herein include stearic acid, calcium hydroxide, talc, corn starch, sodium stearyl fumarate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include sugars (including lactose, sucrose, and dextrose), polysaccharides (including dextrates and maltodextrin), polyols (including mannitol, xylitol, and sorbitol), cyclodextrins and the like.

Non-water-soluble diluents are compounds typically used in the formulation of pharmaceuticals, such as calcium phosphate, calcium sulfate, starches, modified starches and microcrystalline cellulose, and micro cellulose (for example, having a density of about 0.45 g/cm³, for example Avicel®, powdered cellulose), and talc.

Suitable wetting agents for use in the solid dosage forms described herein include oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (for example, Polyquat 10®), sodium oleate, sodium lauryl sulfate, magnesium stearate, sodium docusate, triacetin, vitamin E TPGS and the like. Wetting agents include surfactants.

Suitable surfactants for use in the solid dosage forms described herein include docusate and its pharmaceutically acceptable salts, sodium lauryl sulfate, sorbitan monooleate, polyoxyethylene sorbitan monooleate, polysorbates, poloxamers, bile salts, glyceryl monostearate, copolymers of ethylene oxide and propylene oxide, for example, Pluronic® (BASF), and the like.

Suitable suspending agents for use in the solid dosage forms described here include polyvinylpyrrolidone, for example, polyvinylpyrrolidone K12, polyvinylpyrrolidone K17, polyvinylpyrrolidone K25, or polyvinylpyrrolidone K30, polyethylene glycol, for example, the polyethylene glycol can have a molecular weight of about 300 to about 6000, or about 3350 to about 4000, or about 7000 to about 18000, vinylpyrrolidone/vinyl acetate copolymer (S630), sodium alginate, gums, such as, for example, gum tragacanth and gum acacia, guar gum, xanthans, including xanthan gum, sugars, cellulosic, such as, for example, sodium carboxymethylcellulose, methylcellulose, sodium carboxymethylcellulose, hydroxypropylmethylcellulose, hydroxyethylcellulose, polysorbate-80, polyethoxylated sorbitan monolaurate, polyethoxylated sorbitan monolaurate, povidone and the like.

Suitable antioxidants for use in the solid dosage forms described herein include, for example, butylated hydroxytoluene (BHT), butyl hydroxyanisole (BHA), sodium ascorbate, Vitamin E TPGS, ascorbic acid, sorbic acid and tocopherol.

Immediate-release formulations may be prepared by combining superdisintegrants such as Croscarmellose sodium and different grades of microcrystalline cellulose in different ratios. To aid disintegration, sodium starch glycolate will be added.

The above-listed additives should be taken as merely examples and not limiting, of the types of additives that can be included in solid dosage forms of the present invention. The amounts of such additives can be readily determined by one skilled in the art, according to the particular properties desired.

Oral liquid dosage forms include solutions, emulsions, suspensions, and syrups. These oral liquid dosage forms may be formulated with any pharmaceutically acceptable excipient known to those of skill in the art for the preparation of liquid dosage forms. For example, water, glycerin, simple syrup, alcohol, and combinations thereof.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as but not limited to, an oil, water, an alcohol, and combinations of these pharmaceutically suitable surfactants, suspending agents, emulsifying agents, may be added for oral or parenteral administration. Suspensions may include oils. Such oils include peanut oil, sesame oil, cottonseed oil, corn oil, and olive oil. Suspension preparation may also contain esters of fatty acids such as ethyl oleate, isopropyl myristate, fatty acid glycerides, and acetylated fatty acid glycerides. Suspension formulations may include alcohols, such as ethanol, isopropyl alcohol, hexadecyl alcohol, glycerol, and propylene glycol. Ethers, such as poly(ethylene glycol), petroleum hydrocarbons such as mineral oil and petrolatum, and water may also be used in suspension formulations.

In some embodiments, formulations are provided comprising particles of a compound described herein and at least one dispersing agent or suspending agent for oral administration to a subject in need thereof.

The formulation may be a powder and/or granules for suspension, and upon admixture with water, a substantially uniform suspension is obtained. As described herein, the aqueous dispersion can comprise amorphous and non-amorphous particles consisting of multiple effective particle sizes such that the drug is absorbed in a controlled manner over time. In certain embodiments, the aqueous dispersion or suspension is an immediate-release formulation. In another embodiment, an aqueous dispersion comprising amorphous particles is formulated such that a portion of the particles of the present invention are absorbed within, for example, about 0.75 hours after administration and the remaining particles are absorbed 2 to 4 hours after absorption of the earlier particles.

In other embodiments, addition of a complexing agent to the aqueous dispersion results in a larger span of the particles to extend the drug absorption phase of the active agent such that 50-80% of the particles are absorbed in the first hour and about 90% are absorbed by about 4 hours. Dosage forms for oral administration can be aqueous suspensions selected from the group including pharmaceutically acceptable aqueous oral dispersions, emulsions, solutions, and syrups. See, for example, Singh et al., Encyclopedia of Pharm. Tech., 2nd Ed., 754-757 (2002). In addition to the active agents of the present invention particles, the liquid dosage forms may comprise additives, such as (a) disintegrating agents; (b) dispersing agents; (c) wetting agents; (d) at least one preservative; (e) viscosity enhancing agents; (f) at least one sweetening agent; and (g) at least one flavoring agent.

Examples of disintegrating agents for use in the aqueous suspensions and dispersions include a starch, for example, a natural starch such as corn starch or potato starch, a pregelatinized starch such as National 1551 or Amijel®, or sodium starch glycolate such as Promogel® or Explotab®; a cellulose such as a wood product, microcrystalline cellulose, for example, Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crosspovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropylcellulose ethers (for example, HPC, HPC-SL, and HPC-L), hydroxypropylmethylcellulose and hydroxypropylmethylcellulose ethers (for example HPMC K100, HPMC K4M, HPMC K15M, and HPMC K100M), carboxymethylcellulose sodium, methylcellulose, hydroxyethylcellulose, hydroxypropylmethylcellulose phthalate, hydroxypropylmethylcellulose acetate stearate, noncrystalline cellulose, magnesium aluminum silicate, triethanolamine, polyvinyl alcohol (PVA), polyvinylpyrrolidone/vinyl acetate copolymer (Plasdone®, for example, S-630), 4-(1,1,3,3-tetramethylbutyl)-phenol polymer with ethylene oxide and formaldehyde (also known as tyloxapol), poloxamers (for example, Pluronics F68®, F88®, and F108®, which are block copolymers of ethylene oxide and propylene oxide); and poloxamines (for example, Tetronic 908®, also known as Poloxamine 908®, which is a tetrafunctional block copolymer derived from sequential addition of propylene oxide and ethylene oxide to ethylenediamine (BASF Corp., Parsippany, N.J.)).

In other embodiments, the dispersing agent is selected from a group not comprising one of the following agents: hydrophilic polymers; electrolytes; Tween® 60 or 80; PEG; polyvinylpyrrolidone (PVP); hydroxypropyl cellulose and hydroxypropyl cellulose ethers (for example, HPC, HPC-SL, and HPC-L); hydroxypropyl methylcellulose and hydroxypropyl methylcellulose ethers (for example HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethylcellulose phthalate; hydroxypropylmethylcellulose acetate stearate; non-crystalline cellulose; magnesium aluminum silicate; triethanolamine; polyvinyl alcohol (PVA); 4-(1,1,3,3-tetramethyl butyl)-phenol polymer with ethylene oxide and formaldehyde; poloxamers (for example, Pluronics F68®, F88®, and F108®, which are block copolymers of ethylene oxide and propylene oxide); or poloxamines (for example, Tetronic 908® or Poloxamine 908®).

Wetting agents (including surfactants) suitable for the aqueous suspensions and dispersions described herein are known in the art and include cetyl alcohol, glycerol monostearate, polyoxyethylene sorbitan fatty acid esters (for example, the commercially available Tweens® such as for example, Tween 20® and Tween 80® (ICI Specialty Chemicals)), and polyethylene glycols (for example, Carbowaxs 3350® and 1450®, and Carpool 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphatidylcholine and the like.

Suitable preservatives for the aqueous suspensions or dispersions described herein include potassium sorbate, parabens (for example, methylparaben and propylparaben) and their salts, benzoic acid and its salts, other esters of para hydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

In one embodiment, the aqueous liquid dispersion can comprise methylparaben and propylparaben in a concentration ranging from at least about 0.01% to about 0.3% or less methylparaben by weight to the weight of the aqueous dispersion and at least about 0.005% to about 0.03% or less propylparaben by weight to the total aqueous dispersion weight. In yet another embodiment, the aqueous liquid dispersion can comprise methylparaben from at least about 0.05 to about 0.1 or less weight % and propylparaben from at least about 0.01 to about 0.02 or less weight % of the aqueous dispersion.

Suitable viscosity enhancing agents for the aqueous suspensions or dispersions described herein include methyl cellulose, xanthan gum, carboxymethylcellulose, hydroxypropyl cellulose, hydroxypropylmethyl cellulose, Plasdone® S-630, carbomer, polyvinyl alcohol, alginates, acacia, chitosans and combinations thereof. The concentration of the viscosity-enhancing agent will depend upon the agent selected and the viscosity desired.

In addition to the additives listed above, the liquid formulations of the present invention can also comprise inert diluents commonly used in the art, such as water or other solvents, solubilizing agents, emulsifiers, and/or sweeteners.

In one embodiment, the formulation for oral delivery is an effervescent powder containing a compound described herein or a pharmaceutically acceptable salt thereof. In one embodiment, the formulation for oral delivery is an effervescent powder containing a compound described herein or a pharmaceutically acceptable salt thereof. Effervescent salts have been used to disperse medicines in water for oral administration. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the present invention are added to water, the acids and the base react to liberate carbon dioxide gas, thereby causing “effervescence.” Examples of effervescent salts include sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

Tablets of the invention described here can be prepared by methods well known in the art. Various methods for the preparation of the immediate release, modified release, controlled release, and extended-release dosage forms (for example, as matrix tablets, tablets having one or more modified, controlled, or extended-release layers, etc.) and the vehicles therein are well known in the art. Generally recognized compendia of methods include: Remington: The Science and Practice of Pharmacy, Alfonso R. Gennaro, Editor, 20th Edition, Lippincott Williams & Wilkins, Philadelphia, PA; and Sheth et al. (1980), Compressed tablets, in Pharmaceutical dosage forms, Vol. 1, edited by Lieberman and Lachtman, Dekker, NY.

In certain embodiments, solid dosage forms, for example tablets, effervescent tablets, and capsules, are prepared by mixing the active agents of the present invention particles with one or more pharmaceutical excipients to form a bulk blend composition. When referring to these bulk blend compositions as homogeneous, it is meant that the active agents of the present invention particles are dispersed evenly throughout the composition so that the composition may be readily subdivided into equally effective unit dosage forms, such as tablets, pills, and capsules. The individual unit dosages may also comprise film coatings, which disintegrate upon oral ingestion or upon contact with diluents. These the active agents of the present invention formulations can be manufactured by conventional pharmaceutical techniques.

Conventional pharmaceutical techniques for preparation of solid dosage forms include, for example, one or a combination of methods: (1) dry mixing, (2) direct compression, (3) milling, (4) dry or non-aqueous granulation, (5) wet granulation, or (6) fusion. See, for example, Lachman et al., Theory and Practice of Industrial Pharmacy (1986). Other methods include, for example, spray drying, pan coating, melt granulation, granulation, fluidized bed spray drying or coating (for example, Wurster coating), tangential coating, top spraying, tableting, extruding and the like.

Compressed tablets are solid dosage forms prepared by compacting the bulk blend the active agents of the present invention formulations described above. In various embodiments, compressed tablets which are designed to dissolve in the mouth will comprise one or more flavoring agents. In other embodiments, the compressed tablets will comprise a film surrounding a final compressed tablet. In some embodiments, the film coating can provide a delayed release of the active agents of the present invention formulation. In other embodiments, the film coating aids in patient compliance (for example, Opadry® coatings or sugar coating). Film coatings comprising Opadry® typically range from about 1% to about 3% of the tablet weight. Film coatings for delayed-release usually comprise 2-6% of a tablet weight or 7-15% of a spray-layered bead weight. In other embodiments, the compressed tablets comprise one or more excipients.

A capsule may be prepared, for example, by placing the bulk blend of the active agents of the present invention formulation, described above, inside of a capsule. In some embodiments, the formulations of the present invention (non-aqueous suspensions and solutions) are placed in a soft gelatin capsule. In other embodiments, the formulations of the present invention are placed in standard gelatin capsules or non-gelatin capsules such as capsules comprising HPMC. In other embodiments, the formulations of the present invention are placed in a sprinkle capsule, wherein the capsule may be swallowed whole, or the capsule may be opened, and the contents sprinkled on food prior to eating. In some embodiments of the present invention, the therapeutic dose is split into multiple (for example, two, three, or four) capsules. In some embodiments, the entire dose of the active agents of the present invention is delivered in a capsule form.

In certain embodiments, ingredients (including or not including the active agent) of the invention are wet granulated. The individual steps in the wet granulation process of tablet preparation include milling and sieving of the ingredients, dry powder mixing, wet massing, granulation, drying, and final grinding. In various embodiments, the active agents of the present invention composition are added to the other excipients of the pharmaceutical formulation after they have been wet granulated. Alternatively, the ingredients may be subjected to dry granulation, for example, via compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). No wet binder or moisture is involved in any of the steps.

In some embodiments, the active agents of the present invention formulation are dry granulated with other excipients in the pharmaceutical formulation. In other embodiments, the active agents of the present invention formulation are added to other excipients of the pharmaceutical formulation after they have been dry granulated.

In other embodiments, the formulation of the present invention formulations described herein is a solid dispersion. Methods of producing such solid dispersions are known in the art and include U.S. Pat. Nos. 4,343,789; 5,340,591; 5,456,923; 5,700,485; 5,723,269; and U.S. Pub. No. 2004/0013734. In some embodiments, the solid dispersions of the invention comprise both amorphous and non-amorphous active agents of the present invention and can have enhanced bioavailability as compared to conventional active agents of the present invention formulations. In still other embodiments, the active agents of the present invention formulations described herein are solid solutions. Solid solutions incorporate a substance together with the active agent and other excipients such that heating the mixture results in the dissolution of the drug and the resulting composition is then cooled to provide a solid blend that can be further formulated or directly added to a capsule or compressed into a tablet.

### Extended-Release Formulations

Depending on the desired release profile, the pharmaceutical formulation, for example, an oral solid dosage form, may contain a suitable amount of controlled-release agents, extended-release agents, and/or modified-release agents (for example, delayed-release agents). The pharmaceutical solid oral dosage forms comprising the active agents of the present invention described herein can be further formulated to provide a modified or controlled release of the active agents of the present invention. In some embodiments, the solid dosage forms described herein can be formulated as a delayed release dosage form such as an enteric-coated delayed release oral dosage forms, i.e., as an oral dosage form of a pharmaceutical composition as described herein which uses an enteric coating to affect release in the small intestine of the gastrointestinal tract. The enteric-coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated. Enteric coatings may also be used to prepare other controlled release dosage forms including extended-release and pulsatile release dosage forms.

In other embodiments, the active agents of the formulations described herein are delivered using a pulsatile dosage form. Pulsatile dosage forms comprising the active agents of the present invention described herein may be administered using a variety of formulations known in the art. For example, such formulations include those described in U.S. Pat. Nos. 5,011,692; 5,017,381; 5,229,135; and 5,840,329. Other dosage forms suitable for use with the active agents of the present invention are described in, for example, U.S. Pat. Nos. 4,871,549; 5,260,068; 5,260,069; 5,508,040; 5,567,441; and 5,837,284.

In one embodiment, the controlled release dosage form is pulsatile release solid oral dosage form comprising at least two groups of particles, each containing active agents of the present invention as described herein. The first group of particles provides a substantially immediate dose of the active agents of the present invention upon ingestion by a subject. The first group of particles can be either uncoated or comprise a coating and/or sealant. The second group of particles comprises coated particles, which may comprise from at least about 2% to about 75% or less, typically from at least about 2.5% to about 70% or less, or from at least about 40% to about 70% or less, by weight of the total dose of the active agents of the present invention in the formulation, in admixture with one or more binders.

In one embodiment, a coating for providing a controlled, delayed, or extended-release is applied to a compound described herein or to a core containing a compound described herein.

The coating may comprise a pharmaceutically acceptable ingredient in an amount sufficient, for example, to provide an extended release from for example, about 1 hours to about 7 hours following ingestion before release of the active agent. Suitable coatings include one or more differentially degradable coatings such as, by way of example only, pH-sensitive coatings (enteric coatings) such as acrylic resins (for example, Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, and Eudragit® NE30D, Eudragit® NE 40D®) either alone or blended with cellulose derivatives, for example, ethylcellulose, or non-enteric coatings having variable thickness to provide differential release of the active agents of the present invention formulation.

Many other types of controlled/delayed/extended-release systems known to those of ordinary skill in the art and are suitable for use with the active agents of the present invention formulations described herein. Examples of such delivery systems include polymer-based systems, such as polylactic and polyglycolic acid, polyanhydrides and polycaprolactone, cellulose derivatives (for example, ethylcellulose), porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, for example, Liberman et al., Pharmaceutical Dosage Forms, 2 Ed., Vol. 1, pp. 209-214 (1990); Singh et al., Encyclopedia of Pharmaceutical Technology, 2nd Ed., pp. 751-753 (2002); U.S. Pat. Nos. 4,327,725; 4,624,848; 4,968,509; 5,461,140; 5,456,923, 5,516,527; 5,622,721, 5,686,105; 5,700,410; 5,977,175; 6,465,014 and 6,932,983.

In certain embodiments, the controlled release systems may comprise the controlled/delayed/extended-release material incorporated with the drug(s) into a matrix, whereas in other formulations, the controlled release material may be applied to a core containing the drug(s). In certain embodiments, one drug may be incorporated into the core while the other drug is incorporated into the coating. In some embodiments, materials include shellac, acrylic polymers, cellulosic derivatives, polyvinyl acetate phthalate, and mixtures thereof. In other embodiments, materials include Eudragit® series E, L, RL, RS, NE, L, L300, S, 100-55, cellulose acetate phthalate, Aquateric, cellulose acetate trimellitate, ethyl cellulose, hydroxypropylmethylcellulose phthalate, hydroxypropylmethylcellulose acetate succinate, polyvinyl acetate phthalate, and Cotteric.

The controlled/delayed/extended-release systems may use a hydrophilic polymer, including a water-swellable polymer (for example, a natural or synthetic gum). The hydrophilic polymer may be any pharmaceutically acceptable polymer which swells and expands in the presence of water to slowly release the active agents of the present invention. These polymers include polyethylene oxide, methylcellulose, hydroxypropyl cellulose, hydroxypropylmethylcellulose, and the like.

The performance of acrylic polymers (primarily their solubility in biological fluids) can vary based on the degree and type of substitution. Examples of suitable acrylic polymers which may be used in matrix formulations or coatings include methacrylic acid copolymers and ammonia methacrylate copolymers. The Eudragit series E, L, S, RL, RS and NE (Rohm Pharma) are available as solubilized in an organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in the stomach and dissolve in the intestine; Opadry Enteric is also insoluble in the stomach and dissolves in the intestine.

Examples of suitable cellulose derivatives for use in matrix formulations or coatings include ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous-based system and is a spray-dried CAP psuedolatex with particles<1 μm. Other components in Aquateric can include pluronic, Tweens, and acetylated monoglycerides. Other suitable cellulose derivatives include cellulose acetate trimellitate (Eastman); methylcellulose (Pharmacoat, Methocel); hydroxypropylmethylcellulose phthalate (HPMCP); hydroxypropylmethylcellulose succinate (HPMCS); and hydroxypropylmethylcellulose acetate succinate (for example, AQOAT (Shin Etsu)). The performance can vary based on the degree and type of substitution. For example, HPMCP such as, HP-50, HP-55, HP-555, HP-55F grades are suitable. The performance can vary based on the degree and type of substitution. For example, suitable grades of hydroxypropylmethylcellulose acetate succinate include AS-LG (LF), which dissolves at pH 5, AS-MG (MF), which dissolves at pH 5.5, and AS-HG (HF), which dissolves at higher pH. These polymers are offered as granules or as fine powders for aqueous dispersions. Other suitable cellulose derivatives include hydroxypropylmethylcellulose.

In some embodiments, the coating may contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate, and triacetin. Conventional coating techniques such as spray or pan coating are employed to apply coatings. The coating thickness must be sufficient to ensure that the oral dosage form remains intact until the desired site of topical delivery in the intestinal tract is reached.

Multilayer tablet delivery (for example, such as that used in the GeoMatrix™ technology) comprises a hydrophilic matrix core containing the active ingredient and one or two impermeable or semi-permeable polymeric coatings. This technology uses films or compressed polymeric barrier coatings on one or both sides of the core. The presence of polymeric coatings (for example, such as that used in the GeoMatrix™ technology) modifies the hydration/swelling rates of the core and reduces the surface area available for drug release. These partial coatings provide modulation of the drug dissolution profile: they reduce the release rate from the device and shift the typical time-dependent release rate toward constant release. This technology enables customized levels of controlled release of specific active agents and/or simultaneous release of two different active agents at different rates that can be achieved from a single tablet. The combination of layers, each with different rates of swelling, gelling and erosion, is used for the rate of drug release in the body. Exposure of the multilayer tablet as a result of the partial coating may affect the release and erosion rates, therefore, transformation of a multilayered tablet with exposure on all sides to the gastrointestinal fluids upon detachment of the barrier layer will be considered.

Multi-layered tablets containing combinations of immediate release and modified/extended release of two different active agents or dual release rate of the same drug in a single dosage form may be prepared by using hydrophilic and hydrophobic polymer matrices. Dual release repeat action multi-layered tablets may be prepared with an outer compression layer with an initial dose of rapidly disintegrating matrix in the stomach and a core inner layer tablet formulated with components that are insoluble in the gastric media but release efficiently in the intestinal environment.

In certain embodiments, the dosage form is a solid oral dosage form which is an immediate release dosage form whereby >80% of the active agents of the present invention are released within 2 hours after administration. In other embodiments, the invention provides an (for example, solid oral) dosage form that is a controlled release or pulsatile release dosage form. In such instances, the release may be, for example, 30 to 60% of the active agents of the present invention particles by weight are released from the dosage form within about 2 hours after administration and about 90% by weight of the active agents of the present invention released from the dosage form, for example, within about 4 hours after administration. In yet other embodiments, the dosage form includes at least one active agent in an immediate-release form and at least one active agent in the delayed-release form or sustained-release form. In yet other embodiments, the dosage form includes at least two active agents that are released at different rates as determined by in-vitro dissolution testing or via oral administration.

The various release dosage formulations discussed above, and others known to those skilled in the art can be characterized by their disintegration profile. A profile is characterized by the test conditions selected. Thus, the disintegration profile can be generated at a pre-selected apparatus type, shaft speed, temperature, volume, and pH of the dispersion media. Several disintegration profiles can be obtained. For example, a first disintegration profile can be measured at a pH level approximating that of the stomach (about pH 1.2); a second disintegration profile can be measured at a pH level approximating that of one point in the intestine or several pH levels approximating multiple points in the intestine (about 6.0 to about 7.5, more specifically, about 6.5 to 7.0). Another disintegration profile can be measured using distilled water. The release of formulations may also be characterized by their pharmacokinetic parameters, for example, Cmax, Tₘₐₓ, and AUC (0-τ).

In certain embodiments, the controlled, delayed or extended-release of one or more of the active agents of the fixed-dose combinations of the invention may be in the form of a capsule having a shell comprising the material of the rate-limiting membrane, including any of the coating materials previously discussed, and filled with the active agents of the present invention particles. A particular advantage of this configuration is that the capsule may be prepared independently of the active agent of the present invention particles; thus, process conditions that would adversely affect the drug can be used to prepare the capsule.

Alternatively, the formulation may comprise a capsule having a shell made of a porous or a pH-sensitive polymer made by a thermal forming process. Another alternative is a capsule shell in the form of an asymmetric membrane, i.e., a membrane that has a thin skin on one surface and most of whose thickness is constituted of a highly permeable porous material. The asymmetric membrane capsules may be prepared by a solvent exchange phase inversion, wherein a solution of polymer, coated on a capsule-shaped mold, is induced to phase separate by exchanging the solvent with a miscible non-solvent. In another embodiment, spray layered active agents of the present invention particles are filled in a capsule.

An exemplary process for manufacturing the spray layered the active agents of the present invention is the fluidized bed spraying process. The active agents of the present invention suspensions or the active agents of the present invention complex suspensions described above may be sprayed onto sugar or microcrystalline cellulose (MCC) beads (20-35 mesh) with Wurster column insert at an inlet temperature of 50° C. to 60° C. and air temp of 30° C. to 50° C. A 15 to 20 wt % total solids content suspension containing 45 to 80 wt % the active agents of the present invention, 10 to 25 wt % hydroxymethylpropylcellulose, 0.25 to 2 wt % of SLS, 10 to 18 wt % of sucrose, 0.01 to 0.3 wt % simethicone emulsion (30% emulsion) and 0.3 to 10% NaCl, based on the total weight of the solid content of the suspension, are sprayed (bottom spray) onto the beads through 1.2 mm nozzles at 10 mL/min and 1.5 bar of pressure until a layering of 400 to 700% wt % is achieved as compared to initial beads weight. The resulting spray layered the active agents of the present invention particles, or the active agents of the present invention complex particles comprise about 30 to 70 wt % of the active agents of the present invention based on the total weight of the particles.

In one embodiment the capsule is a size 0 soft gelatin capsule. In one embodiment, the capsule is a swelling plug device. In another embodiment, the swelling plug device is further coated with cellulose acetate phthalate or copolymers of methacrylic acid and methylmethacrylate. In some embodiments, the capsule includes at least 40 mg (or at least 100 mg or at least 200 mg) of the active agents of the present invention and has a total weight of less than 800 mg (or less than 700 mg). The capsule may contain a plurality of the active agents of the present invention-containing beads, for example, spray layered beads. In some embodiments, the beads are 12-25% the active agents of the present invention by weight. In some embodiments, some or all of the active agents of the present invention containing beads are coated with a coating comprising 6 to 15% (or 8 to 12%) of the total bead weight. Optimization work typically involves lower loading levels, and the beads constitute 30 to 60% of the finished bead weight. The capsule may contain a granulated composition, wherein the granulated composition comprises the active agents of the present invention.

The capsule may provide pulsatile release of the active agents of the present invention oral dosage form. In one embodiment, the formulations comprise: (a) a first dosage unit comprising a compound described herein that is released substantially immediately following oral administration of the dosage form to a patient; (b) a second dosage unit comprising a compound described herein that is released approximately 2 to 6 hours following administration of the dosage form to a patient.

In one embodiment, the formulation comprises: (a) a first dosage unit comprising a compound described herein that is released substantially immediately following oral administration of the dosage form to a patient; (b) a second dosage unit comprising a compound described herein that is released approximately 2 to 6 hours following administration of the dosage form to a patient.

For pulsatile release capsules containing beads, the beads can be coated with a coating comprising 6 to 15% (or 8 to 12%) of the total bead weight. In some embodiments, the coating is a coating that is insoluble at pH 1 to 2 and soluble at pH greater than 5.5. In other embodiments, the pulsatile release capsule contains a plurality of beads formulated for modified release and the at least one agent of the present invention is, for example, spray granulated for immediate release.

In some embodiments, the release of the active agents of the present invention particles can be modified with a modified release coating, such as an enteric coating using cellulose acetate phthalate or a sustained release coating comprising copolymers of methacrylic acid and methylmethacrylate. In one embodiment, the enteric coating may be present in an amount of about 0.5 to about 15 wt %, more specifically, about 8 to about 12 wt %, based on the weight of, for example, the spray layered particles. In one embodiment, the spray layered particles coated with the delayed and/or sustained release coatings can be filled in a modified release capsule in which both enteric-coated particles and immediate release particles of the present invention beads are filled into a soft gelatin capsule. Additional suitable excipients may also be filled with the coated particles in the capsule. The uncoated particles release the active agent of the present invention immediately upon administration while the coated particles do not release the active agent of the present invention until these particles reach the intestine. By controlling the ratios of the coated and uncoated particles, desirable pulsatile release profiles also may be obtained. In some embodiments, the ratios between the uncoated and the coated particles are for example, 20/80, or 30/70, or 40/60, or 50/50, w/w to obtain desirable release.

In certain embodiments, spray layered active agents of the present invention can be compressed into tablets with commonly used pharmaceutical excipients. Any appropriate apparatus for forming the coating can be used to make the enteric coated tablets, for example, fluidized bed coating using a Wurster column, powder layering in coating pans or rotary coaters; dry coating by double compression technique; tablet coating by film coating technique, and the like. See, for example, U.S. Pat. No. 5,322,655; Remington's Pharmaceutical Sciences Handbook: Chapter 90 “Coating of Pharmaceutical Dosage Forms,” 1990.

In certain embodiments, the spray layered active agents of the present invention described above and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the active agents of the present invention formulation into the gastrointestinal fluid. In other embodiments, the spray layered active agents of the present invention particles or spray layered active agents complex particles with enteric coatings described above and one or more excipients are dry blended and compressed into a mass, such as a tablet.

In certain embodiments, a pulsatile release of the active agent of the present invention formulation comprises a first dosage unit comprising a formulation made from the active agent of the present invention containing granules made from a spray drying or spray granulated procedure or a formulation made from the active agent of the present invention complex containing granules made from a spray drying or spray granulated procedure without enteric or sustained-release coatings and a second dosage unit comprising spray layered the active agent of the present invention particles or spray layered the active agent of the present invention complex particles with enteric or sustained-release coatings. In one embodiment, the active agent is wet or dry blended and compressed into a mass to make a pulsatile release tablet.

In certain embodiments, binding, lubricating and disintegrating agents are blended (wet or dry) to the spray layered active agent of the present invention to make a compressible blend. In one embodiment, the dosage unit containing a compound described herein and the dosage unit containing the other pharmacological agent are compressed separately and then compressed together to form a bilayer tablet. In yet another embodiment, the dosage unit containing the other pharmacological agent is in the form of an overcoat and completely covers the second dosage unit containing a compound described herein. In yet another embodiment, the dosage unit containing a compound described herein is in the form of an overcoat and completely covers the second dosage unit containing the other pharmacological agent.

In one embodiment, the dosage unit containing a compound of any of a compound described herein and the dosage unit containing the other pharmacological agent are compressed separately and then compressed together to form a bilayer tablet. In yet another embodiment, the dosage unit containing the other pharmacological agent is in the form of an overcoat and completely covers the second dosage unit containing a compound described herein. In yet another embodiment, the dosage unit containing a compound described herein is in the form of an overcoat and completely covers the second dosage unit containing the other pharmacological agent.

### Systemic Formulations

The formulations of the present invention can include any selected compound of the present invention for any of the disclosed indications in a form suitable for intramuscular, subcutaneous, or intravenous injection may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Additionally, the active agents of the present invention can be dissolved at concentrations of greater than about 1 mg/ml using water-soluble beta cyclodextrins (for example, beta-sulfobutyl-cyclodextrin and 2-hydroxypropyl-beta-cyclodextrin. Proper fluidity can be maintained, for example, by the use of a coating such as a lecithin, by the maintenance of the required particle size in the case of dispersions, and by the use of surfactants.

The formulations of the present invention suitable for subcutaneous injection may also contain additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, sorbic acid, and the like. It may also be desirable to include isotonic agents, such as sugars, sodium chloride, and the like. Prolonged drug absorption of the injectable pharmaceutical form can be brought about by the use of agents delaying absorption, such as aluminum monostearate and gelatin. The formulations of the present invention designed for extended-release via subcutaneous or intramuscular injection can avoid first-pass metabolism and lower dosages of the active agents of the present invention will be necessary to maintain plasma levels of about 50 ng/ml. In such formulations, the particle size of the active agents of the present invention and the range of the particle sizes of the active agents of the present invention particles can be used to control the release of the drug by controlling the rate of dissolution in fat or muscle.

In one embodiment, a pharmaceutical composition contain a compound described herein or a pharmaceutically acceptable salt thereof is formulated into a dosage form suitable for parenteral use. In one embodiment, pharmaceutical compositions containing compounds of any of a compound described herein or a pharmaceutically acceptable salt thereof is formulated into a dosage form suitable for parenteral use. The dosage form may be selected from, but not limited to, a lyophilized powder, a solution, or a suspension (for example, a depot suspension).

In one embodiment, a pharmaceutical composition containing a combination of the present invention or a pharmaceutically acceptable salt thereof is formulated into a topical dosage form. In one embodiment, a pharmaceutical composition containing a compound of any of combination of the present invention or a pharmaceutically acceptable salt thereof is formulated into a topical dosage form. The topical dosage form is selected from, but not limited to, a patch, a gel, a paste, a cream, an emulsion, a liniment, a balm, a lotion, and an ointment.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. Such patches may be constructed for continuous, pulsatile, or on demand delivery of pharmaceutical agents.

Frequently, it will be desirable or necessary to introduce the pharmaceutical composition to the brain, either directly or indirectly. Direct techniques usually involve placement of a drug delivery catheter into the host's ventricular system to bypass the blood-brain barrier. Indirect techniques, which are generally useful, usually involve formulating the compositions to provide for drug latentiation by the conversion of hydrophilic drugs into lipid-soluble drugs or prodrugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

### Non-Limiting Examples of Formulations for Systemic Delivery

The examples below provide non-limiting embodiments of formulations, which can be used to deliver any of the compounds described herein in enantiomerically enriched form, pure form or even a racemic mixture. Therefore, while the compounds below are specified, any desired purity form or compound can be used if it achieves the desired goal of treatment.

### Pharmaceutically Acceptable Salts

The compounds described herein, including enantiomerically enriched mixtures, can be administered if desired as a pharmaceutically acceptable salt or a salt mixture. A salt mixture may be useful to increase solubility of the active substances, to alter pharmacokinetics, or for controlled release or other objective. A salt mixture may comprise 2, 3, 4, 5, 6, or more pharmaceutically acceptable salts together to form a single composition.

The compounds of the present invention are amines and thus basic, and therefore, react with inorganic and organic acids to form pharmaceutically acceptable acid addition salts. In some embodiments, the compounds of the present invention as free amines are oily and have decreased stability at room temperature. In this case it may be beneficial to convert the free amines to their pharmaceutically acceptable acid addition salts for ease of handling and administration because in some embodiments, the pharmaceutically acceptable salt is solid at room temperature.

Acids commonly employed to form such salts are inorganic acids such as hydrochloric acid, hydrobromic acid, hydroiodic acid, sulfuric acid, nitric acid, phosphoric acid, and the like, and organic acids, such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. In one embodiment, the compounds of the present invention are administered as oxalate salts. In one embodiment of the present invention, the compounds are administered as phosphate salts.

Exemplary salts include, but are not limited to, 2-hydroxyethanesulfonate, 2-naphthalenesulfonate, 3-hydroxy-2-naphthoate, 3-phenylpropionate, acetate, adipate, alginate, amsonate, aspartate, benzenesulfonate, benzoate, besylate, bicarbonate, bisulfate, bitartrate, borate, butyrate, calcium edetate, camphorate, camphorsulfonate, camsylate, carbonate, citrate, clavulariate, cyclopentanepropionate, digluconate, dodecylsulfate, edetate, edisylate, estolate, esylate, ethanesulfonate, finnarate, gluceptate, glucoheptanoate, gluconate, glutamate, glycerophosphate, glycollylarsanilate, hemisulfate, heptanoate, hexafluorophosphate, hexanoate, hexylresorcinate, hydrabamine, hydrobromide, hydrochloride, hydroiodide, hydroxynaphthoate, iodide, sethionate, lactate, lactobionate, laurate, laurylsulphonate, malate, maleate, mandelate, mesylate, methanesulfonate, methylbromide, methylnitrate, methylsulfate, mucate, naphthylate, napsylate, nicotinate, nitrate, N-methylglucamine ammonium salt, oleate, oxalate, palmitate, pamoate, pantothenate, pectinate, persulfate, phosphate, phosphateldiphosphate, picrate, pivalate, polygalacturonate, propionate, p-toluenesulfonate, saccharate, salicylate, stearate, subacetate, succinate, sulfate, sulfosaliculate, suramate, tannate, tartrate, teoclate, thiocyanate, tosylate, triethiodide, undecanoate, and valerate salts, and the like.

Alternatively, exemplary salts include 2-hydroxyethanesulfonate, 2-naphthalenesulfonate, 2-napsylate, 3-hydroxy-2-naphthoate, 3-phenylpropionate, 4-acetamidobenzoate, acefyllinate, acetate, aceturate, adipate, alginate, aminosalicylate, ammonium, amsonate, ascorbate, aspartate, benzenesulfonate, benzoate, besylate, bicarbonate, bisulfate, bitartrate, borate, butyrate, calcium edetate, calcium, camphocarbonate, camphorate, camphorsulfonate, camsylate, carbonate, cholate, citrate, clavulariate, cyclopentanepropionate, cypionate, d-aspartate, d-camsylate, d-lactate, decanoate, dichloroacetate, digluconate, dodecylsulfate, edentate, edetate, edisylate, estolate, esylate, ethanesulfonate, ethyl sulfate, fumarate, furate, fusidate, galactarate (mucate), galacturonate, gallate, gentisate, gluceptate, glucoheptanoate, gluconate, glucuronate, glutamate, glutarate, glycerophosphate, glycolate, glycollylarsanilate, hemisulfate, heptanoate (enanthate), heptanoate, hexafluorophosphate, hexanoate, hexylresorcinate, hippurate, hybenzate, hydrabamine, hydrobromide, hydrobromide/bromide, hydrochloride, hydroiodide, hydroxide, hydroxybenzoate, hydroxynaphthoate, iodide, isethionate, isothionate, 1-aspartate, 1-camsylate, 1-lactate, lactate, lactobionate, laurate, laurylsulphonate, lithium, magnesium, malate, maleate, malonate, mandelate, meso-tartrate, mesylate, methanesulfonate, methylbromide, methylnitrate, methylsulfate, mucate, myristate, N-methylglucamine ammonium salt, napadisilate, naphthylate, napsylate, nicotinate, nitrate, octanoate, oleate, orotate, oxalate, p-toluenesulfonate, palmitate, pamoate, pantothenate, pectinate, persulfate, phenylpropionate, phosphate, phosphateldiphosphate, picrate, pivalate, polygalacturonate, potassium, propionate, pyrophosphate, saccharate, salicylate, salicylsulfate, sodium, stearate, subacetate, succinate, sulfate, sulfosaliculate, sulfosalicylate, suramate, tannate, tartrate, teoclate, terephthalate, thiocyanate, thiosalicylate, tosylate, tribrophenate, triethiodide, undecanoate, undecylenate, valerate, valproate, xinafoate, zinc, and the like. (See Berge et al. (1977) “Pharmaceutical Salts,” J. Pharm. Sci. 66:1-19.) Pharmaceutically acceptable salts include those employing a hydrochloride anion.

### EXAMPLES

### Example 1: Fixed Dose Combination Tablets of 30/70 mg R-MDMA, S-MDMA

A fixed dose combination tablet is formulated to contain 30 mg of R-MDMA (as HCl salt), and 70 mg of S-MDMA (as HCl salt). The tablet is formulated and manufactured in order to achieve a modified release profile consisting of immediate release R-MDMA.HCl, shortly thereafter followed by immediate release of S-MDMA.HCl (for example release of S-MDMA.HCl about 15 minutes after dosing, retarding of the majority of the release onset compared to R-MDMA). The product is formulated and processed in a manner to achieve the stated objectives of the release characteristics for the majority, but not the entirety, of the release timing of each active ingredient.

### Step 1.1: R-MDMA Granules

A 1 kg granular blend of R-MDMA.HCl with excipients is prepared as follows:
1. According to the composition and quantities listed in Table 1.1, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 30 mg of R-MDMA (parent compound, regardless of salt content or salt form) per 100 mg of granular sample material. The material is temporarily stored in a sealed LDPE bag and labeled as R-MDMA granules.

TABLE 1.1
composition of R-MDMA granules

| | | | | Quantity/tablet, |
|---|---|---|---|---|
| Component | Function | Quantity % | Quantity, g | mg |
| R-MDMA•HCl | active | 30 | 300 | 30 |
| Microcrystalline cellulose^{a} | non-brittle filler | 63.8 | 638 | 63.8 |
| Croscarmellose sodium | super-disintegrant | 5 | 50 | 5 |
| Magnesium stearate | lubricant | 1.2 | 12 | 1.2 |
| Totals | | 100% | 1,000 g | 100 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 1.2: S-MDMA Granules

A 2 kg granular blend of S-MDMA.HCl with excipients is prepared as follows: 1. According to the composition and quantities listed in Table 1.2, the active ingredient, and
excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 70 mg of S-MDMA (parent compound, regardless of salt form or salt content) per 200 mg of granular sample material. The material is temporarily stored in a sealed LDPE bag and labeled as S-MDMA granules.

TABLE 1.2
composition of S-MDMA granules

| | | | | Quantity/tablet, |
|---|---|---|---|---|
| Component | Function | Quantity % | Quantity, g | mg |
| S-MDMA•HCl | active | 35 | 700 | 70 |
| Microcrystalline cellulose^{a} | non-brittle filler | 63.2 | 1,264 | 126.4 |
| Croscarmellose sodium | super-disintegrant | 0.4 | 8 | 0.8 |
| Magnesium stearate | lubricant | 1.4 | 28 | 2.8 |
| Totals | | 100% | 2,000 g | 200 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 1.3: Bilayer Tablet Compression

A 3 kg batch of bilayer tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 1.3, the two phases containing active ingredients from Steps 1.1, and 1.2, are loaded to the individual feed hoppers of a bilayer rotary tablet press
2. The press is set up such that one layer of the tablet contains 100 mg of the R-MDMA granules and 200 mg of the S-MDMA granules. These layer weights targets are approximate and controlled within pharmaceutically acceptable ranges of variability.
3. The tablet press is operated to produce bilayer tablets of 300 mg total core weight, containing 30 mg of R-MDMA, and 70 mg of S-MDMA (parent compounds regardless of salt form or salt content)
4. The core tablets are coated with a cosmetic (non-functional) coating to a weight gain of about 3% w/w using an aqueous spray, fully perforated, pan film coater. The coating material is an Opadry II white coating system (available from Colorcon) containing PVA polymer, PEG, talc, and titanium dioxide pigment.
5. After the film coating weight target is achieved within an acceptable range of variability, the spray system is stopped, and the drying system is continued with periodic slow movement of the coating pan for an additional 10-20 minutes to remove excess water from the tablets. When the coated tablets reach an acceptable residual moisture content determined by at-line testing with a loss on drying moisture balance, the coating process is stopped and the coated tablets are discharged from the coating machine.

TABLE 1.3
composition of tablets

| | | | | Quantity/tablet, |
|---|---|---|---|---|
| Component | Function | Quantity % | Quantity, g | mg |
| R-MDMA granules | Active | 33.3 | 1,000 | 100 |
| S-MDMA granules | Active | 66.7 | 2,000 | 200 |
| Opadry II White film coating | Cosmetic coating | 3 | 90 | 9 |
| Totals | | 103% | 3,090 g | 309 mg |

### Example 2: Fixed Dose Combination Tablets of 10/110/25 m2 Dextroamphetamine, S-MDMA, Milnacipran

A fixed dose combination tablet is formulated to contain 10 mg of dextroamphetamine sulfate, 110 mg of S-MDMA.HCl, and 25 mg of milnacipran hydrochloride. The tablet is formulated and manufactured in order to achieve a complex modified release profile consisting of immediate release dextroamphetamine, shortly thereafter followed by immediate release of S-MDMA.HCl (for example release of S-MDMA.HCl about 15 minutes after dosing retarding of the majority of the release onset compared to dextroamphetamine); then followed by the delayed release of milnacipran hydrochloride. For example, the release of milnacipran hydrochloride about 3 hours post dosing. The product is formulated and processed in a manner to achieve the stated objectives of the release characteristics for the majority, but not the entirety, of the release timing of each active ingredient.

### Step 2.1: Dextroamphetamine Granules

A 1 kg granular blend of dextroamphetamine sulfate with excipients is prepared as follows:
1. According to the composition and quantities listed in Table 2.1, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.5 mm.
4. The resulting granules are tested and confirmed to have a potency of about 10 mg of dextroamphetamine (parent compound) per 100 mg of granular sample material. The material is temporarily stored in a sealed LDPE bag and labeled as dextroamphetamine granules.

TABLE 2.1
composition of granules

| | | | | Quantity/tablet, |
|---|---|---|---|---|
| Component | Function | Quantity % | Quantity, g | mg |
| Dextroamphetamine sulfate | active | 10 | 100 | 10 |
| Microcrystalline cellulose^{a} | non-brittle filler | 85 | 850 | 85 |
| Croscarmellose sodium | super-disintegrant | 4 | 40 | 4 |
| Magnesium stearate | lubricant | 1 | 10 | 1 |
| Totals | | 100% | 1,000 g | 100 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 2.2: S-MDMA Granules

A 2.75 kg granular blend of S-MDMA.HCl with excipients is prepared as follows:
1. According to the composition and quantities listed in Table 2.2, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.5 mm.
4. The resulting granules are tested and confirmed to have a potency of about 110 mg of S-MDMA (parent compound) per 275 mg of granular sample material. The material is temporarily stored in a sealed LDPE bag and labeled as S-MDMA granules.

TABLE 2.2
composition of S-MDMA granules

| | | | | Quantity/tablet, |
|---|---|---|---|---|
| Component | Function | Quantity % | Quantity, g | mg |
| S-MDMA•HCl | active | 40 | 1,110 | 110 |
| Microcrystalline cellulose^{a} | non-brittle filler | 58 | 1,595 | 159.5 |
| Croscarmellose sodium | super-disintegrant | 0.5 | 13.75 | 1.375 |
| Magnesium stearate | lubricant | 1.5 | 41.25 | 4.125 |
| Totals | | 100% | 2,750 g | 275 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 2.3: Milnacipran Enteric Beads

A quantity of 2.5 kg of enteric protected beads of milnacipran hydrochloride is prepared as follows:
1. According to the composition and quantities listed in Table 2.3, the active ingredient and enteric coating system are dissolved/dispersed in water to a combined concentration of about 20% w/w.
2. The dispersion is spray coated onto sugar spheres in a fluid bed coater operated in Wurster coating mode, and dried in place, yielding beads coated with active drug and enteric protective polymer materials.
3. The resulting beads are tested to confirm acid resistance and confirmed to have a potency of about 25 mg of milnacipran (parent compound) per 250 mg of beads. The material is temporarily stored in a sealed LDPE bag and labeled as milnacipran enteric beads.

TABLE 2.3
composition of milnacipran enteric granules

| | | | | Quantity/tablet, |
|---|---|---|---|---|
| Component | Function | Quantity % | Quantity, g | mg |
| Milnacipran•HCl | active | 10 | 250 | 25 |
| Eudragit FL 30-D^{a} | enteric polymer coating | 40 | 1,000 | 100 |
| Sugar spheres | carrier particles | 50 | 1,250 | 125 |
| Totals | | 100% | 2,500 g | 250 mg |

^{a}note the actual mass of the active ingredient is increased such that the quantity given corresponds to the content of the parent molecule (excluding the mass contribution of the salt form counterion and any other impurities present). Concomitantly, the mass of Eudragit is decreased by the same amount that the active ingredient is increased.

### Step 2.4: Final Blending and Tablet Compression

A 10 kg final blend and tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 2.4, the three phases containing active ingredients from Steps 2.1, 2.2, and 2.3 and as well as the other listed excipients (excluding the lubricant) are blended in a V-shell blender for about 5 minutes at about 25 rpm.
2. The lubricant is added to the blender and blending is continued for an additional about 2 minutes at about 25 rpm.
3. The resulting final blend is tested for blend uniformity to confirm potencies of about 10 mg dextroamphetamine (parent compound), about 110 mg of S-MDMA (parent compound), and about 25 mg of milnacipran (parent compound): each per 1,000 mg of final blend.
4. The final blend is transferred to a rotary tablet press and compressed to form tablets containing 10/110/25 mg respectively of the parent compounds dextroamphetamine, S-MDMA, and milnacipram

TABLE 2.4
composition of tablets

| | | | | Quantity/tablet, |
|---|---|---|---|---|
| Component | Function | Quantity % | Quantity, g | mg |
| Dextroamphetamine granules | Active | 10 | 1,000 | 100 |
| S-MDMA granules | Active | 27.5 | 2,750 | 275 |
| Milnacipram enteric beads | Active | 25 | 2,500 | 250 |
| Microcrystalline cellulose | non-brittle filler | 35.5 | 3,550 | 355 |
| Croscarmellose sodium | super-disintegrant | 1.5 | 150 | 15 |
| Magnesium stearate | lubricant | 0.5 | 50 | 5 |
| Totals | | 100% | 10,000 g | 1,000 mg |

### Example 3: Fixed Dose Combination Tablets of 10/80 mg Dextroamphetamine, S-5-MAPB

A fixed dose combination tablet is formulated to contain 10 mg of dextroamphetamine (as sulfate salt), and 80 mg of S-5-MAPB (as HCl salt). The tablet is formulated and manufactured to achieve a modified release profile consisting of immediate release dextroamphetamine, shortly thereafter followed by immediate release of S-5-MAPB.HCl (for example release of S-5-MAPB.HCl about 15 minutes after dosing, retarding of the majority of the release onset compared to dextroamphetamine). The product is formulated and processed in a manner to achieve the stated objectives of the release characteristics for the majority, but not the entirety, of the release timing of each active ingredient.

### Step 3.1: Dextroamphetamine Granules

A 1 kg granular blend of dextroamphetamine sulfate with excipients is prepared following the description given in Example 2, step 2.1

### Step 3.2: S-5-MAPB Granules

A 4 kg granular blend of S-5-MAPB.HCl with excipients is prepared as follows:
1. According to the composition and quantities listed in Table 3.1, the active ingredient, and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 80 mg of S-5-MAPB (parent compound, regardless of salt form or salt content) per 400 mg of granular sample material. The material is temporarily stored in a sealed LDPE bag and labeled as S-5-MAPB granules.

TABLE 3.1
composition of S-5-MAPB granules

| | | | | Quantity/tablet, |
|---|---|---|---|---|
| Component | Function | Quantity % | Quantity, g | mg |
| S-5-MAPB•HCl | Active | 20 | 800 | 80 |
| Microcrystalline cellulose^{a} | non-brittle filler | 78.5 | 3,140 | 314 |
| Croscarmellose sodium | super-disintegrant | 0.5 | 20 | 2 |
| Magnesium stearate | lubricant | 1 | 40 | 4 |
| Totals | | 100% | 4,000 g | 400 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 3.1: Bilayer Tablet Compression

A 5 kg batch of bilayer tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 3.2, the two phases containing active ingredients from Steps 3.1, and 3.2, are loaded to the individual feed hoppers of a bilayer rotary tablet press
2. The press is set up such that one layer of the tablet contains 100 mg of the dextroamphetamine granules and 400 mg of the S-5-MAPB granules. These layer weights targets are approximate and controlled within pharmaceutically acceptable ranges of variability.
3. The tablet press is operated to produce bilayer tablets of 500 mg total core weight, containing 10 mg of dextroamphetamine, and 80 mg of S-5-MAPB (parent compounds regardless of salt form or salt content)
4. The core tablets are coated with a cosmetic (non-functional) coating to a weight gain of about 4% w/w using an aqueous spray, fully perforated, pan film coater. The coating material is an Opadry II Swedish Orange coating system (available from Colorcon) containing HPMC polymer, PEG, talc, and inorganic oxide pigments to achieve opacity and colored appearance.
5. After the film coating weight target is achieved within an acceptable range of variability, the spray system is stopped, and the drying system is continued with periodic slow movement of the coating pan for an additional 10-20 minutes to remove excess water from the tablets. When the coated tablets reach an acceptable residual moisture content determined by at-line testing with a loss on drying moisture balance, the coating process is stopped, and the coated tablets are discharged from the coating machine.

TABLE 3.2
composition of tablets

| | | | | Quantity/tablet, |
|---|---|---|---|---|
| Component | Function | Quantity % | Quantity, g | mg |
| Dextroamphetamine granules | Active | 20 | 1,000 | 100 |
| S-5-MAPB granules | Active | 80 | 4,000 | 400 |
| Opadry II Swedish Orange film | Cosmetic | 4 | 200 | 20 |
| coating | coating | | | |
| Totals | | 104% | 5,200 g | 520 mg |

### Example 4: Fixed Dose Combination Tablets of 10/80/25 m2 Dextroamphetamine, S-5-MAPB, Milnacipran

A fixed dose combination tablet is formulated to contain 10 mg of dextroamphetamine sulfate, 80 mg of S-5-MAPB.HCl, and 25 mg of milnacipran hydrochloride. The tablet is formulated and manufactured in order to achieve a complex modified release profile consisting of immediate release dextroamphetamine, shortly thereafter followed by immediate release of S-5-MAPB.HCl (for example release of S-5-MAPB.HCl about 15 minutes after dosing, retarding of the majority of the release onset compared to dextroamphetamine); then followed by the delayed release of milnacipran hydrochloride. For example, the release of milnacipran hydrochloride about 3 hours post dosing. The product is formulated and processed in a manner to achieve the stated objectives of the release characteristics for the majority, but not the entirety, of the release timing of each active ingredient.

### Step 4.1: Dextroamphetamine Granules

A 1 kg granular blend of dextroamphetamine sulfate with excipients is prepared following the description given in Example 2, step 2.1

### Step 4.2: S-5-MAPB Granules

A 4 kg granular blend of S-5-MAPB.HCl with excipients is prepared following the description given in Example 3, step 3.2

### Step 4.3: Milnacipran Enteric Beads

A quantity of 2.5 kg of enteric protected beads of milnacipran hydrochloride is prepared following the description given in Example 2, Step 2.3

### Step 4.4: Final Blending and Tablet Compression

A 10 kg final blend and tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 4.1, the three phases containing active ingredients from Steps 4.1, 4.2, and 4.3 and as well as the other listed excipients (excluding the lubricant) are blended in a V-shell blender for about 5 minutes at about 25 rpm.
2. The lubricant is added to the blender and blending is continued for an additional about 2 minutes at about 25 rpm.
3. The resulting final blend is tested for blend uniformity to confirm potencies of about 10 mg dextroamphetamine (parent compound), about 80 mg of S-5-MAPB (parent compound), and about 25 mg of milnacipran (parent compound): each per 1,000 mg of final blend.
4. The final blend is transferred to a rotary tablet press and compressed to form tablets containing 10/80/25 mg respectively of the parent compounds dextroamphetamine, S-5-MAPB, and milnacipram

TABLE 4.1
composition of tablets

| | | | | Quantity/tablet, |
|---|---|---|---|---|
| Component | Function | Quantity % | Quantity, g | mg |
| Dextroamphetamine granules | Active | 10 | 1,000 | 100 |
| S-5-MAPB granules | Active | 40 | 4,000 | 400 |
| Milnacipram enteric beads | Active | 25 | 2,500 | 250 |
| Microcrystalline cellulose | non-brittle filler | 23 | 2,300 | 230 |
| Croscarmellose sodium | super-disintegrant | 1.5 | 150 | 15 |
| Magnesium stearate | lubricant | 0.5 | 50 | 5 |
| Totals | | 100% | 10,000 g | 1,000 mg |

### Example 5: Fixed Dose Combination Tablets of 80/100 mg R-BK-MDMA, S-BK-MDMA

A fixed dose combination tablet is formulated to contain 80 mg of R-BK-MDMA, and 100 mg of S-BK-MDMA (each as hydrochloride salts). The tablet is formulated and manufactured in order to achieve a complex modified release profile consisting of immediate and sustained release of R-BK-MDMA (for example, over approximately a 90-minute period) and shortly after initial release of R-BK-MDMA, a sustained release initiates of S-BK-MDMA, also continuing for approximately 90 minutes. The product is formulated and processed in a manner to achieve the stated objectives of the release characteristics for the majority, but not the entirety, of the release timing of each active ingredient.

### Step 5.1: R-BK-MDMA Granules

A 4 kg granular blend of R-BK-MDMA.HCl with excipients is prepared as follows:
1. According to the composition and quantities listed in Table 5.1, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 80 mg of R-BK-MDMA (parent compound, regardless of salt content or salt form) per 400 mg of granular sample material. The material is temporarily stored in a sealed LDPE bag and labeled as R-BK-MDMA granules.

TABLE 5.1
composition of R-BK-MDMA granules

| | | | | Quantity/tablet, |
|---|---|---|---|---|
| Component | Function | Quantity % | Quantity, g | mg |
| R-BK-MDMA•HCl | active | 20 | 800 | 80 |
| Microcrystalline cellulose^{a} | non-brittle filler | 67 | 2,680 | 268 |
| Hypromellose | hydrogel former | 5 | 200 | 20 |
| Sodium Lauryl Sulfate | surfactant | 2 | 80 | 8 |
| Croscarmellose sodium | super-disintegrant | 5 | 200 | 20 |
| Magnesium stearate | lubricant | 1 | 40 | 4 |
| Totals | | 100% | 4,000 g | 400 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 5.2: S-BK-MDMA Granules

A 5 kg granular blend of S-BK-MDMA.HCl with excipients is prepared as follows:
1. According to the composition and quantities listed in Table 5.2, the active ingredient, and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 100 mg of S-BK-MDMA (parent compound, regardless of salt form or salt content) per 500 mg of granular sample material. The material is temporarily stored in a sealed LDPE bag and labeled as S-BK-MDMA granules.

TABLE 5.2
composition of S-BK-MDMA granules

| | | | | Quantity/tablet, |
|---|---|---|---|---|
| Component | Function | Quantity % | Quantity, g | mg |
| S-BK-MDMA•HCl | active | 20 | 1,000 | 100 |
| Microcrystalline cellulose^{a} | non-brittle filler | 71.5 | 3,575 | 357.5 |
| Hypromellose | hydrogel former | 5 | 250 | 25 |
| Sodium Lauryl Sulfate | surfactant | 2 | 100 | 10 |
| Croscarmellose sodium | super-disintegrant | 0.5 | 25 | 2.5 |
| Magnesium stearate | lubricant | 1 | 50 | 5 |
| Totals | | 100% | 5,000 g | 500 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 5.3: Bilayer Tablet Compression

A 9 kg batch of bilayer tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 5.3, the two phases containing active ingredients from Steps 5.1, and 5.2, are loaded to the individual feed hoppers of a bilayer rotary tablet press
2. The press is set up such that one layer of the tablet contains 400 mg of the R-BK-MDMA granules and 500 mg of the S-BK-MDMA granules. These layer weights targets are approximate and controlled within pharmaceutically acceptable ranges of variability.
3. The tablet press is operated to produce bilayer tablets of 900 mg total core weight, containing 80 mg of R-BK-MDMA, and 100 mg of S-BK-MDMA (parent compounds regardless of salt form or salt content)
4. The core tablets are coated with a cosmetic (non-functional) coating to a weight gain of about 3% w/w using an aqueous spray, fully perforated, pan film coater. The coating material is an Opadry II white coating system (available from Colorcon) containing PVA polymer, PEG, talc, and titanium dioxide pigment.
5. After the film coating weight target is achieved within an acceptable range of variability, the spray system is stopped, and the drying system is continued with periodic slow movement of the coating pan for an additional 10-20 minutes to remove excess water from the tablets. When the coated tablets reach an acceptable residual moisture content determined by at-line testing with a loss on drying moisture balance, the coating process is stopped, and the coated tablets are discharged from the coating machine.

TABLE 5.3
composition of tablets

| | | | | Quantity/tablet, |
|---|---|---|---|---|
| Component | Function | Quantity % | Quantity, g | mg |
| R-BK-MDMA granules | Active | 44.4 | 4,000 | 400 |
| S-BK-MDMA granules | Active | 55.6 | 5,000 | 900 |
| Opadry II White film coating | Cosmetic coating | 3 | 270 | 27 |
| Totals | | 103% | 9,270 g | 927 mg |

### Example 6: Fixed Dose Combination Tablets of 60/40 m2 S-5-APB, R-5-APB

A fixed dose combination tablet is formulated to contain 60 mg of S-5-APB (as HCl salt), and 40 mg of R-5-APB (as HCl salt). The tablet is formulated and manufactured in order to achieve a modified release profile consisting of immediate release S-5-APB, shortly thereafter followed by immediate release of R-5-APB (for example release of R-5-APB about 15 minutes after dosing, retarding of the majority of the release onset compared to S-5-APB). The product is formulated and processed in a manner to achieve the stated objectives of the release characteristics for the majority, but not the entirety, of the release timing of each active ingredient.

### Step 6.1: S-5-APB Granules

A 3 kg granular blend of S-5-APB.HCl with excipients is prepared as follows:
1. According to the composition and quantities listed in Table 6.1, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 60 mg of S-5-APB (parent compound, regardless of salt content or salt form) per 300 mg of granular sample material. The material is temporarily stored in a sealed LDPE bag and labeled as S-5-APB granules.

TABLE 6.1
composition of S-5-APB granules

| | | | | Quantity/tablet, |
|---|---|---|---|---|
| Component | Function | Quantity % | Quantity, g | mg |
| S-5-APB•HCl | active | 20 | 600 | 60 |
| Microcrystalline cellulose^{a} | non-brittle filler | 73.8 | 2,214 | 221.4 |
| Croscarmellose sodium | super-disintegrant | 5 | 150 | 15 |
| Magnesium stearate | lubricant | 1.2 | 36 | 3.6 |
| Totals | | 100% | 3,000 g | 300 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 6.2: R-5-APB Granules

A 2 kg granular blend of R-5-APB.HCl with excipients is prepared as follows:
1. According to the composition and quantities listed in Table 6.2, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 40 mg of R-5-APB (parent compound, regardless of salt content or salt form) per 200 mg of granular sample material. The material is temporarily stored in a sealed LDPE bag and labeled as R-5-APB granules.

TABLE 6.2
composition of R-5-APB granules

| | | | | Quantity/tablet, |
|---|---|---|---|---|
| Component | Function | Quantity % | Quantity, g | mg |
| R-5-APB•HCl | active | 20 | 400 | 40 |
| Microcrystalline cellulose^{a} | non-brittle filler | 73.8 | 1,476 | 147.6 |
| Croscarmellose sodium | super-disintegrant | 5 | 100 | 10 |
| Magnesium stearate | lubricant | 1.2 | 24 | 2.4 |
| Totals | | 100% | 2,000 g | 200 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 6.3: Bilayer Tablet Compression

A 5 kg batch of bilayer tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 6.3, the two phases containing active ingredients from Steps 6.1, and 6.2, are loaded to the individual feed hoppers of a bilayer rotary tablet press
2. The press is set up such that one layer of the tablet contains 300 mg of the S-5-APB granules and 200 mg of the R-5-APB granules. These layer weights targets are approximate and controlled within pharmaceutically acceptable ranges of variability.
3. The tablet press is operated to produce bilayer tablets of 500 mg total core weight, containing 60 mg of S-5-APB, and 40 mg of R-5-APB (parent compounds regardless of salt form or salt content)
4. The core tablets are coated with a cosmetic (non-functional) coating to a weight gain of about 3% w/w using an aqueous spray, fully perforated, pan film coater. The coating material is an Opadry II Yellow coating system (available from Colorcon) containing PVA polymer, PEG, talc, and pigments.
5. After the film coating weight target is achieved within an acceptable range of variability, the spray system is stopped, and the drying system is continued with periodic slow movement of the coating pan for an additional 10-20 minutes to remove excess water from the tablets. When the coated tablets reach an acceptable residual moisture content determined by at-line testing with a loss on drying moisture balance, the coating process is stopped, and the coated tablets are discharged from the coating machine.

TABLE 6.3
composition of tablets

| | | | | Quantity/tablet, |
|---|---|---|---|---|
| Component | Function | Quantity % | Quantity, g | mg |
| S-5-APB granules | Active | 60 | 3,000 | 300 |
| R-5-APB granules | Active | 40 | 2,000 | 200 |
| Opadry II Yellow film coating | Cosmetic coating | 3 | 150 | 15 |
| Totals | | 103% | 5,150 g | 515 mg |

### Example 7: Fixed Dose Combination Tablets of 60/40 m2 S-6-APB, R-6-APB

A fixed dose combination tablet is formulated to contain 60 mg of S-6-APB (as HCl salt), and 40 mg of R-6-APB (as HCl salt). The tablet is formulated and manufactured in order to achieve a modified release profile consisting of immediate release S-6-APB, shortly thereafter followed by immediate release of R-5-APB (for example release of R-6-APB about 15 minutes after dosing, retarding of the majority of the release onset compared to S-6-APB). The product is formulated and processed in a manner to achieve the stated objectives of the release characteristics for the majority, but not the entirety, of the release timing of each active ingredient.

### Step 7.1: S-6-APB Granules

A 3 kg granular blend of S-6-APB.HCl with excipients is prepared as follows:
1. According to the composition and quantities listed in Table 7.1, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 60 mg of S-6-APB (parent compound, regardless of salt content or salt form) per 300 mg of granular sample material. The material is temporarily stored in a sealed LDPE bag and labeled as S-6-APB granules.

TABLE 7.1
composition of S-6-APB granules

| | | | | Quantity/tablet, |
|---|---|---|---|---|
| Component | Function | Quantity % | Quantity, g | mg |
| S-6-APB•HCl | active | 20 | 600 | 60 |
| Microcrystalline cellulose^{a} | non-brittle filler | 73.8 | 2,214 | 221.4 |
| Croscarmellose sodium | super-disintegrant | 5 | 150 | 15 |
| Magnesium stearate | lubricant | 1.2 | 36 | 3.6 |
| Totals | | 100% | 3,000 g | 300 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 7.2: R-6-APB Granules

A 2 kg granular blend of R-6-APB.HCl with excipients is prepared as follows:
1. According to the composition and quantities listed in Table 7.2, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 40 mg of R-6-APB (parent compound, regardless of salt content or salt form) per 200 mg of granular sample material. The material is temporarily stored in a sealed LDPE bag and labeled as R-6-APB granules.

TABLE 7.2
composition of R-6-APB granules

| | | | | Quantity/tablet, |
|---|---|---|---|---|
| Component | Function | Quantity % | Quantity, g | mg |
| R-6-APB•HCl | active | 20 | 400 | 40 |
| Microcrystalline cellulose^{a} | non-brittle filler | 73.8 | 1,476 | 147.6 |
| Croscarmellose sodium | super-disintegrant | 5 | 100 | 10 |
| Magnesium stearate | lubricant | 1.2 | 24 | 2.4 |
| Totals | | 100% | 2,000 g | 200 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 7.3: Bilayer Tablet Compression

A 5 kg batch of bilayer tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 7.3, the two phases containing active ingredients from Steps 7.1, and 7.2, are loaded to the individual feed hoppers of a bilayer rotary tablet press
2. The press is set up such that one layer of the tablet contains 300 mg of the S-6-APB granules and 200 mg of the R-6-APB granules. These layer weights targets are approximate and controlled within pharmaceutically acceptable ranges of variability.
3. The tablet press is operated to produce bilayer tablets of 500 mg total core weight, containing 60 mg of S-6-APB, and 40 mg of R-6-APB (parent compounds regardless of salt form or salt content)
4. The core tablets are coated with a cosmetic (non-functional) coating to a weight gain of about 3% w/w using an aqueous spray, fully perforated, pan film coater. The coating material is an Opadry II Blue coating system (available from Colorcon) containing PVA polymer, PEG, talc, and pigments.
5. After the film coating weight target is achieved within an acceptable range of variability, the spray system is stopped, and the drying system is continued with periodic slow movement of the coating pan for an additional 10-20 minutes to remove excess water from the tablets. When the coated tablets reach an acceptable residual moisture content determined by at-line testing with a loss on drying moisture balance, the coating process is stopped, and the coated tablets are discharged from the coating machine.

TABLE 7.3
composition of tablets

| | | | | Quantity/tablet, |
|---|---|---|---|---|
| Component | Function | Quantity % | Quantity, g | mg |
| S-6-APB granules | Active | 60 | 3,000 | 300 |
| R-6-APB granules | Active | 40 | 2,000 | 200 |
| Opadry II Blue film coating | Cosmetic coating | 3 | 150 | 15 |
| Totals | | 103% | 5,150 g | 515 mg |

### Example 8: Fixed Dose Combination Tablets of 20/40 mg R-BK-5-MAPB, S-BK-5-MAPB

A fixed dose combination tablet is formulated to contain 20 mg of R-BK-5-MAPB, and 40 mg of S-BK-5-MAPB (each as hydrochloride salts). The tablet is formulated and manufactured in order to achieve a complex modified release profile consisting of immediate and sustained release of R-BK-5-MAPB (for example, over approximately a 90-minute period) and shortly after initial release of R-BK-5-MAPB, a sustained release initiates of S-BK-5-MAPB, also continuing for approximately 90 minutes. The product is formulated and processed in a manner to achieve the stated objectives of the release characteristics for the majority, but not the entirety, of the release timing of each active ingredient.

### Step 8.1: R-BK-5-MAPB Granules

A 2 kg granular blend of R-BK-5-MAPB.HCl with excipients is prepared as follows:
1. According to the composition and quantities listed in Table 8.1, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 20 mg of R-BK-MDMA (parent compound, regardless of salt content or salt form) per 200 mg of granular sample material. The material is temporarily stored in a sealed LDPE bag and labeled as R-BK-5-MAPB granules.

TABLE 8.1
composition of R-BK-5-MAPB granules

| | | | | Quantity/tablet, |
|---|---|---|---|---|
| Component | Function | Quantity % | Quantity, g | mg |
| R-BK-5-MAPB•HCl | active | 10 | 200 | 20 |
| Microcrystalline cellulose^{a} | non-brittle filler | 79 | 1,580 | 158 |
| Hypromellose | hydrogel former | 5 | 100 | 10 |
| Sodium Lauryl Sulfate | surfactant | 2 | 40 | 4 |
| Croscarmellose sodium | super-disintegrant | 3 | 60 | 6 |
| Magnesium stearate | lubricant | 1 | 20 | 2 |
| Totals | | 100% | 2,000 g | 200 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 8.2: S-BK-5-MAPB Granules

A 4 kg granular blend of S-BK-5-MAPB.HCl with excipients is prepared as follows:
1. According to the composition and quantities listed in Table 8.2, the active ingredient, and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 40 mg of S-BK-MDMA (parent compound, regardless of salt form or salt content) per 400 mg of granular sample material. The material is temporarily stored in a sealed LDPE bag and labeled as S-BK-5-MAPB granules.

TABLE 8.2
composition of S-BK-5-MAPB granules

| | | | | Quantity/tablet, |
|---|---|---|---|---|
| Component | Function | Quantity % | Quantity, g | mg |
| S-BK-5-MAPB•HCl | active | 10 | 400 | 40 |
| Microcrystalline cellulose^{a} | non-brittle filler | 81.75 | 3,270 | 327 |
| Hypromellose | hydrogel former | 5 | 200 | 20 |
| Sodium Lauryl Sulfate | surfactant | 2 | 80 | 8 |
| Croscarmellose sodium | super-disintegrant | 0.25 | 10 | 1 |
| Magnesium stearate | lubricant | 1 | 40 | 4 |
| Totals | | 100% | 4,000 g | 400 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 8.3: Bilayer Tablet Compression

A 6 kg batch of bilayer tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 8.3, the two phases containing active ingredients from Steps 8.1, and 8.2, are loaded to the individual feed hoppers of a bilayer rotary tablet press
2. The press is set up such that one layer of the tablet contains 200 mg of the R-BK-5-MAPB granules and 400 mg of the S-BK-5-MAPB granules. These layer weights targets are approximate and controlled within pharmaceutically acceptable ranges of variability.
3. The tablet press is operated to produce bilayer tablets of 600 mg total core weight, containing 20 mg of R-BK-5-MAPB, and 40 mg of S-BK-5-MAPB (parent compounds regardless of salt form or salt content)
4. The core tablets are coated with a cosmetic (non-functional) coating to a weight gain of about 3% w/w using an aqueous spray, fully perforated, pan film coater. The coating material is an Opadry II white coating system (available from Colorcon) containing PVA polymer, PEG, talc, and titanium dioxide pigment.
5. After the film coating weight target is achieved within an acceptable range of variability, the spray system is stopped, and the drying system is continued with periodic slow movement of the coating pan for an additional 10-20 minutes to remove excess water from the tablets. When the coated tablets reach an acceptable residual moisture content determined by at-line testing with a loss on drying moisture balance, the coating process is stopped, and the coated tablets are discharged from the coating machine.

TABLE 8.3
composition of tablets

| | | | | Quantity/tablet, |
|---|---|---|---|---|
| Component | Function | Quantity % | Quantity, g | mg |
| R-BK-5-MAPB granules | Active | 33.3 | 2,000 | 200 |
| S-BK-5-MAPB granules | Active | 67.7 | 4,000 | 400 |
| Opadry II White film coating | Cosmetic coating | 3 | 180 | 18 |
| Totals | | 103% | 6,180 g | 618 mg |

### Example 9a: Fixed Dose Combination Tablets of 7/28 mg R-6-MBPB, S-6-MBPB (as HBr Salts)

A fixed dose combination tablet is formulated to contain 7 mg of R-6-MBPB (as HBr salt), and 28 mg of S-6-MPBP (as HBr salt). The tablet is formulated and manufactured in order to achieve a modified release profile consisting of immediate release R-6-MBPB.HBr, shortly thereafter followed by immediate release of S-6-MBPB.HBr (for example release of S-6-MBPB.HBr about 15 minutes after dosing, retarding of the majority of the release onset compared to R-6-MBPB). The product is formulated and processed in a manner to achieve the stated objectives of the release characteristics for the majority, but not the entirety, of the release timing of each active ingredient.

### Step 9.1: R-6-MBPB Granules

A 1 kg granular blend of R-6-MBPB.HBr with excipients is prepared as follows:
1. According to the composition and quantities listed in Table 9.1, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.0 mm.
4. The resulting granules are tested and confirmed to have a potency of about 7 mg of R-6-MBPB (parent compound, regardless of salt content or salt form) per 100 mg of granular sample material. The material is temporarily stored in a sealed LDPE bag and labeled as R-6-MBPB granules.

TABLE 9.1
composition of R-6-MBPB granules

| | | | | Quantity/tablet, |
|---|---|---|---|---|
| Component | Function | Quantity % | Quantity, g | mg |
| R-6-MDMA•HBr | active | 7 | 70 | 7 |
| Microcrystalline cellulose^{a} | non-brittle filler | 89 | 890 | 89 |
| Croscarmellose sodium | super-disintegrant | 3 | 30 | 3 |
| Magnesium stearate | lubricant | 1 | 10 | 1 |
| Totals | | 100% | 1,000 g | 100 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 9.2: S-6-MBPB Granules

A 1 kg granular blend of S-6-MBPB.HBr with excipients is prepared as follows:
1. According to the composition and quantities listed in Table 9.2, the active ingredient, and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.0 mm.
4. The resulting granules are tested and confirmed to have a potency of about 28 mg of S-6-MBPB (parent compound, regardless of salt form or salt content) per 100 mg of granular sample material. The material is temporarily stored in a sealed LDPE bag and labeled as S-6-MBPB granules.

TABLE 9.2
composition of S-6-MBPB granules

| | | | | Quantity/tablet, |
|---|---|---|---|---|
| Component | Function | Quantity % | Quantity, g | mg |
| S-6-MBPB•HBr | active | 28 | 280 | 28 |
| Microcrystalline cellulose^{a} | non-brittle filler | 70.25 | 702.5 | 70.25 |
| Croscarmellose sodium | super-disintegrant | 0.5 | 5 | 0.5 |
| Magnesium stearate | lubricant | 1.25 | 12.5 | 1.25 |
| Totals | | 100% | 1,000 g | 100 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 9.3: Bilayer Tablet Compression

A 2 kg batch of bilayer tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 9.3, the two phases containing active ingredients from Steps 9.1, and 9.2, are loaded to the individual feed hoppers of a bilayer rotary tablet press
2. The press is set up such that one layer of the tablet contains 100 mg of the R-6-MBPB granules and 100 mg of the S-6-MBPB granules. These layer weights targets are approximate and controlled within pharmaceutically acceptable ranges of variability.
3. The tablet press is operated to produce bilayer tablets of 200 mg total core weight, containing 7 mg of R-6-MBPB, and 28 mg of S-6-MBPB (parent compounds regardless of salt form or salt content)
4. The core tablets are coated with a cosmetic (non-functional) coating to a weight gain of about 3% w/w using an aqueous spray, fully perforated, pan film coater. The coating material is an Opadry Green coating system (available from Colorcon) containing HPMC polymer, PEG, talc, and pigments.
5. After the film coating weight target is achieved within an acceptable range of variability, the spray system is stopped, and the drying system is continued with periodic slow movement of the coating pan for an additional 10-20 minutes to remove excess water from the tablets. When the coated tablets reach an acceptable residual moisture content determined by at-line testing with a loss on drying moisture balance, the coating process is stopped, and the coated tablets are discharged from the coating machine.

TABLE 9.3
composition of tablets

| | | | | Quantity/tablet, |
|---|---|---|---|---|
| Component | Function | Quantity % | Quantity, g | mg |
| R-6-MBPB granules | Active | 50 | 1,000 | 100 |
| S-6-MBPB granules | Active | 50 | 1,000 | 100 |
| Opadry Green film coating | Cosmetic coating | 3 | 60 | 6 |
| Totals | | 103% | 2,060 g | 206 mg |

### Example 9b: Fixed Dose Combination Tablets of 7/28 mg R-6-MBPB, S-6-MBPB (as HCl Salts)

Following the composition, and process as described in Example 9a, the same fixed dose combination product is prepared using the HCl salt forms of R-6-MBPB and S-6-MBPB. All other constituents, quantities, and properties remain equivalent to those of Example 9a.

### Example 10: Fixed Dose Combination Tablets of 12/48 mg R-5-MBPB, S-5-MBPB

A fixed dose combination tablet is formulated to contain 12 mg of R-5-MBPB (as HCl salt), and 48 mg of S-5-MPBP (as HCl salt). The tablet is formulated and manufactured in order to achieve a modified release profile consisting of immediate release R-5-MBPB.HCl, shortly thereafter followed by immediate release of S-5-MBPB.HCl (for example release of S-6-MBPB.HCl about 15 minutes after dosing, retarding of the majority of the release onset compared to R-5-MBPB). The product is formulated and processed in a manner to achieve the stated objectives of the release characteristics for the majority, but not the entirety, of the release timing of each active ingredient.

### Step 10.1: R-6-MBPB Granules

A 1 kg granular blend of R-6-MBPB.HBr with excipients is prepared as follows:
1. According to the composition and quantities listed in Table 10.1, the active ingredient, and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 12 mg of R-5-MBPB (parent compound, regardless of salt content or salt form) per 100 mg of granular sample material. The material is temporarily stored in a sealed LDPE bag and labeled as R-5-MBPB granules.

TABLE 10.1
composition of R-5-MBPB granules

| | | | | Quantity/tablet, |
|---|---|---|---|---|
| Component | Function | Quantity % | Quantity, g | mg |
| R-5-MDMA•HCl | active | 12 | 120 | 12 |
| Microcrystalline cellulose^{a} | non-brittle filler | 83 | 830 | 83 |
| Croscarmellose sodium | super-disintegrant | 4 | 40 | 4 |
| Magnesium stearate | lubricant | 1 | 10 | 1 |
| Totals | | 100% | 1,000 g | 100 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 10.2: S-5-MBPB Granules

A 2 kg granular blend of S-5-MBPB.HCl with excipients is prepared as follows:
1. According to the composition and quantities listed in Table 10.2, the active ingredient, and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 48 mg of S-5-MBPB (parent compound, regardless of salt form or salt content) per 200 mg of granular sample material. The material is temporarily stored in a sealed LDPE bag and labeled as S-5-MBPB granules.

TABLE 10.2
composition of S-5-MBPB granules

| | | | | Quantity/tablet, |
|---|---|---|---|---|
| Component | Function | Quantity % | Quantity, g | mg |
| S-5-MBPB•HCl | active | 24 | 480 | 48 |
| Microcrystalline cellulose^{a} | non-brittle filler | 74.25 | 1,485 | 148.5 |
| Croscarmellose sodium | super-disintegrant | 0.5 | 10 | 1 |
| Magnesium stearate | lubricant | 1.25 | 25 | 2.5 |
| Totals | | 100% | 2,000 g | 200 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 10.3: Bilayer Tablet Compression

A 3 kg batch of bilayer tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 10.3, the two phases containing active ingredients from Steps 10.1, and 10.2, are loaded to the individual feed hoppers of a bilayer rotary tablet press
2. The press is set up such that one layer of the tablet contains 100 mg of the R-5-MBPB granules and 200 mg of the S-5-MBPB granules. These layer weights targets are approximate and controlled within pharmaceutically acceptable ranges of variability.
3. The tablet press is operated to produce bilayer tablets of 300 mg total core weight, containing 12 mg of R-5-MBPB, and 48 mg of S-5-MBPB (parent compounds regardless of salt form or salt content)
4. The core tablets are coated with a cosmetic (non-functional) coating to a weight gain of about 3% w/w using an aqueous spray, fully perforated, pan film coater. The coating material is an Opadry White coating system (available from Colorcon) containing HPMC polymer, PEG, talc, and pigments.
5. After the film coating weight target is achieved within an acceptable range of variability, the spray system is stopped, and the drying system is continued with periodic slow movement of the coating pan for an additional 10-20 minutes to remove excess water from the tablets. When the coated tablets reach an acceptable residual moisture content determined by at-line testing with a loss on drying moisture balance, the coating process is stopped, and the coated tablets are discharged from the coating machine.

TABLE 10.3
composition of tablets

| | | | | Quantity/tablet, |
|---|---|---|---|---|
| Component | Function | Quantity % | Quantity, g | mg |
| R-5-MBPB granules | Active | 33.3 | 1,000 | 100 |
| S-5-MBPB granules | Active | 67.7 | 2,000 | 200 |
| Opadry White film coating | Cosmetic coating | 3 | 90 | 9 |
| Totals | | 103% | 3,090 g | 309 mg |

### Example 11: Fixed Dose Combination Tablets of 60/40/25 m2 S-5-MAPB, RS-5-MAPB, Citalopram

A fixed dose combination tablet is formulated to contain 60 mg of S-5-MAPB sulfate, 40 mg of RS-5-MAPB.HCl, and 25 mg of citalopram hydrobromide. The tablet is formulated and manufactured in order to achieve a complex modified release profile consisting of immediate release S-5-MAPB, shortly thereafter followed by immediate release of RS-5-MAPB.HCl (for example release of RS-5-MAPB.HCl about 15 minutes after dosing, retarding of the majority of the release onset compared to S-5-MAPB); then followed by the delayed release of citalopram hydrobromide. For example, the release of citalopram hydrobromide about 3 hours post dosing. The product is formulated and processed in a manner to achieve the stated objectives of the release characteristics for the majority, but not the entirety, of the release timing of each active ingredient.

### Step 11.1: S-5-MAPB Granules

A 1 kg granular blend of S-5-MAPB sulfate with excipients is prepared as follows:
1. According to the composition and quantities listed in Table 11.1, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.5 mm.
4. The resulting granules are tested and confirmed to have a potency of about 60 mg of S-5-MAPB (parent compound) per 100 mg of granular sample material. The material is temporarily stored in a sealed LDPE bag and labeled as S-5-MAPB granules.

TABLE 11.1
composition of S-5-MAPB granules

| | | | | Quantity/tablet, |
|---|---|---|---|---|
| Component | Function | Quantity % | Quantity, g | mg |
| S-5-MAPB hydrochloride | active | 60 | 600 | 60 |
| Microcrystalline cellulose^{a} | non-brittle filler | 35 | 350 | 35 |
| Croscarmellose sodium | super-disintegrant | 4 | 40 | 4 |
| Magnesium stearate | lubricant | 1 | 10 | 1 |
| Totals | | 100% | 1,000 g | 100 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 11.2: RS-5-MAPB Granules

A 2.75 kg granular blend of RS-5-MAPB.HCl with excipients is prepared as follows:
5. According to the composition and quantities listed in Table 11.2, the active ingredient and excipients (excluding the lubricant) are blended in a V-shell blender for about 10 minutes at about 25 rpm.
6. The lubricant is added to the blender and blending is continued for an additional about 2 minutes at about 25 rpm.
7. The blend is granulated using a roller compactor and oscillating granule mill with a mill screen size of 1.5 mm.
8. The resulting granules are tested and confirmed to have a potency of about 110 mg of RS-5-MAPB (parent compound) per 275 mg of granular sample material. The material is temporarily stored in a sealed LDPE bag and labeled as RS-5-MAPB granules.

TABLE 11.2
composition of RS-5-MAPB granules

| | | | | Quantity/tablet, |
|---|---|---|---|---|
| Component | Function | Quantity % | Quantity, g | mg |
| RS-5-MAPB•HCl | active | 40 | 1,110 | 110 |
| Microcrystalline cellulose^{a} | non-brittle filler | 58 | 1,595 | 159.5 |
| Croscarmellose sodium | super-disintegrant | 0.5 | 13.75 | 1.375 |
| Magnesium stearate | lubricant | 1.5 | 41.25 | 4.125 |
| Totals | | 100% | 2,750 g | 275 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 11.3: Citalopram Enteric Beads

A quantity of 2.5 kg of enteric protected beads of citalopram hydrobromide is prepared as follows:
4. According to the composition and quantities listed in Table 11.3, the active ingredient and enteric coating system are dissolved/dispersed in water to a combined concentration of about 20% w/w.
5. The dispersion is spray coated onto sugar spheres in a fluid bed coater operated in Wurster coating mode, and dried in place, yielding beads coated with active drug and enteric protective polymer materials.
6. The resulting beads are tested to confirm acid resistance and confirmed to have a potency of about 25 mg of citalopram (parent compound) per 250 mg of beads. The material is temporarily stored in a sealed LDPE bag and labeled as citalopram enteric beads.

TABLE 11.3
composition of citalopram enteric granules

| | | | | Quantity/tablet, |
|---|---|---|---|---|
| Component | Function | Quantity % | Quantity, g | mg |
| Citalopram•HBr | active | 10 | 250 | 25 |
| Eudragit FL 30-D^{a} | enteric polymer coating | 40 | 1,000 | 100 |
| Sugar spheres | carrier particles | 50 | 1,250 | 125 |
| Totals | | 100% | 2,500 g | 250 mg |

^{a}note the actual mass of the active ingredient is increased such that the quantity given corresponds to the content of the parent molecule (excluding the mass contribution of the salt form counterion and any other impurities present). Concomitantly, the mass of Eudragit is decreased by the same amount that the active ingredient is increased.

### Step 11.4: Final Blending and Tablet Compression

A 10 kg final blend and tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
5. According to the composition and quantities listed in Table 11.4, the three phases containing active ingredients from Steps 2.1, 2.2, and 2.3 and as well as the other listed excipients (excluding the lubricant) are blended in a V-shell blender for about 5 minutes at about 25 rpm.
6. The lubricant is added to the blender and blending is continued for an additional about 2 minutes at about 25 rpm.
7. The resulting final blend is tested for blend uniformity to confirm potencies of about 60 mg S-5-MAPB (parent compound), about 40 mg of RS-5-MAPB (parent compound), and about 25 mg of citalopram (parent compound): each per 1,000 mg of final blend.
8. The final blend is transferred to a rotary tablet press and compressed to form tablets containing 60/40/25 mg respectively of the parent compounds S-5-MAPB, RS-5-MAPB, and citalopram.

TABLE 11.4
composition of tablets

| | | | | Quantity/tablet, |
|---|---|---|---|---|
| Component | Function | Quantity % | Quantity, g | mg |
| S-5-MAPB granules | Active | 10 | 1,000 | 100 |
| RS-5-MAPB granules | Active | 27.5 | 2,750 | 275 |
| Citalopram enteric beads | Active | 25 | 2,500 | 250 |
| Microcrystalline cellulose | non-brittle filler | 35.5 | 3,550 | 355 |
| Croscarmellose sodium | super-disintegrant | 1.5 | 150 | 15 |
| Magnesium stearate | lubricant | 0.5 | 50 | 5 |
| Totals | | 100% | 10,000 g | 1,000 mg |

### Example 12: Three Component Fixed Dose Combinations

A fixed dose combination tablet is formulated to contain a First Agent, Second Agent, and Third Agent (Agent 1, Agent 2, and Agent 3 respectively). The tablet is formulated and manufactured in order to achieve a complex modified release profile consisting of immediate release First Agent, shortly thereafter followed by immediate release of Second Agent; then followed by the delayed release of Third Agent.

### Step 12.1: Auent 1 Granules

A 1 kg granular blend of Agent 1 with excipients is prepared as follows:
1. According to the composition and quantities listed in Table 12.1, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.5 mm.
4. The resulting granules are tested and confirmed to have the desired potency of Agent 1.

TABLE 12.1
composition of dextroamphetamine granules

| | | | | Quantity/tablet, |
|---|---|---|---|---|
| Component | Function | Quantity % | Quantity, g | mg |
| Agent 1 | active | 10 | 100 | 10 |
| Filler for example | filler | 85 | 850 | 85 |
| Microcrystalline cellulose^{a} | | | | |
| Disintigrant for example | disintegrant | 4 | 40 | 4 |
| Croscarmellose sodium | | | | |
| Lubricant for example | lubricant | 1 | 10 | 1 |
| Magnesium stearate | | | | |
| Totals | | 100% | 1,000 g | 100 mg |

### Step 12.2: Agent 2 Granules

A 2.75 kg granular blend of Agent 2 with excipients is prepared as follows:
1. According to the composition and quantities listed in Table 12.2, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.5 mm.
4. The resulting granules are tested and confirmed to have a potency of the desired potency.

TABLE 12.2
composition of S-MDMA granules

| | | | | Quantity/tablet, |
|---|---|---|---|---|
| Component | Function | Quantity % | Quantity, g | mg |
| Agent 2 | active | 40 | 1,110 | 110 |
| Filler for example | filler | 58 | 1,595 | 159.5 |
| Microcrystalline cellulose^{a} | | | | |
| Disintegrant for example | disintegrant | 0.5 | 13.75 | 1.375 |
| Croscarmellose sodium | | | | |
| Lubricant for example | lubricant | 1.5 | 41.25 | 4.125 |
| Magnesium stearate | | | | |
| Totals | | 100% | 2,750 g | 275 mg |

### Step 12.3: Agent 3 Enteric Beads

A quantity of 2.5 kg of enteric protected beads of Agent 3 is prepared as follows:
1. According to the composition and quantities listed in Table 12.3, the active ingredient and enteric coating system are dissolved/dispersed in water to a combined concentration of about 20% w/w.
2. The dispersion is spray coated onto sugar spheres in a fluid bed coater operated in Wurster coating mode, and dried in place, yielding beads coated with active drug and enteric protective polymer materials.
3. The resulting beads are tested to confirm acid resistance and confirmed to have the desired potency. The material is temporarily stored in a sealed LDPE bag and labeled as milnacipran enteric beads.

TABLE 12.3
composition of milnacipran enteric granules

| | | | | Quantity/tablet, |
|---|---|---|---|---|
| Component | Function | Quantity % | Quantity, g | mg |
| Agent 3 | active | 10 | 250 | 25 |
| Coating for example | enteric polymer coating | 40 | 1,000 | 100 |
| Eudragit FL 30-D^{a} | | | | |
| Carrier for example | carrier particles | 50 | 1,250 | 125 |
| Sugar spheres | | | | |
| Totals | | 100% | 2,500 g | 250 mg |

### Step 12.4: Final Blending and Tablet Compression

A 10 kg final blend and tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 12.4, the three phases containing active ingredients from Steps 12.1, 12.2, and 12.3 and as well as the other listed excipients (excluding the lubricant) are blended in a V-shell blender for about 5 minutes at about 25 rpm.
2. The lubricant is added to the blender and blending is continued for an additional about 2 minutes at about 25 rpm.
3. The resulting final blend is tested for blend uniformity to confirm potencies of the active agents.
4. The final blend is transferred to a rotary tablet press and compressed to form tablets containing the three active agents.

TABLE 12.4
composition of tablets

| | | | | Quantity/tablet, |
|---|---|---|---|---|
| Component | Function | Quantity % | Quantity, g | mg |
| Agent 1 | Active | 10 | 1,000 | 100 |
| Agent 2 | Active | 27.5 | 2,750 | 275 |
| Agent 3 | Active | 25 | 2,500 | 250 |
| Filler for example | filler | 35.5 | 3,550 | 355 |
| Microcrystalline cellulose | | | | |
| Disintegrant for example | disintegrant | 1.5 | 150 | 15 |
| Croscarmellose sodium | | | | |
| Lubricant for example | lubricant | 0.5 | 50 | 5 |
| Magnesium stearate | | | | |
| Totals | | 100% | 10,000 g | 1,000 mg |

### Example 13: Two Component Fixed Dose Formulations

A fixed dose combination tablet is formulated to contain a First Agent and Second Agent (Agent 1 and Agent 2 respectively). The tablet is formulated and manufactured in order to achieve a modified release profile consisting of immediate release Agent 1, shortly thereafter followed by immediate release of Agent 2.

### Step 13.1: R-MDMA Granules

A 1 kg granular blend of Agent 1 with excipients is prepared as follows:
1. According to the composition and quantities listed in Table 13.1, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have the desired potency.

TABLE 13.1
composition of R-MDMA granules

| | | | | Quantity/tablet, |
|---|---|---|---|---|
| Component | Function | Quantity % | Quantity, g | mg |
| Agent 1 | active | 30 | 300 | 30 |
| Filler for example | filler | 63.8 | 638 | 63.8 |
| Microcrystalline cellulose | | | | |
| Disintegrant for example | disintegrant | 5 | 50 | 5 |
| Croscarmellose sodium | | | | |
| Lubricant for example | lubricant | 1.2 | 12 | 1.2 |
| Magnesium stearate | | | | |
| Totals | | 100% | 1,000 g | 100 mg |

### Step 13.2: S-MDMA Granules

A 2 kg granular blend of Agent 2 with excipients is prepared as follows:
1. According to the composition and quantities listed in Table 13.2, the active ingredient, and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed have the desired potency.

TABLE 13.2
composition of S-MDMA granules

| | | | | Quantity/tablet, |
|---|---|---|---|---|
| Component | Function | Quantity % | Quantity, g | mg |
| Agent 2 | active | 35 | 700 | 70 |
| Filler for example | filler | 63.2 | 1,264 | 126.4 |
| Microcrystalline cellulose^{a} | | | | |
| Disintegrant for example | disintegrant | 0.4 | 8 | 0.8 |
| Croscarmellose sodium | | | | |
| Lubricant for example | lubricant | 1.4 | 28 | 2.8 |
| Magnesium stearate | | | | |
| Totals | | 100% | 2,000 g | 200 mg |

### Step 13.3: Bilayer Tablet Compression

A 3 kg batch of bilayer tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 13.3, the two phases containing active ingredients from Steps 13.1, and 13.2, are loaded to the individual feed hoppers of a bilayer rotary tablet press
2. The press is set up such that one layer of the tablet contains the desired amount of Agent 1 granules and another layer of tablet contains the desired amount of Agent 2 granules. These layer weights targets are approximate and controlled within pharmaceutically acceptable ranges of variability.
3. The tablet press is operated to produce bilayer tablets.
4. The core tablets are coated with a cosmetic (non-functional) coating to a weight gain of about 3% w/w using an aqueous spray, fully perforated, pan film coater. The coating material is an Opadry II white coating system (available from Colorcon) containing PVA polymer, PEG, talc, and titanium dioxide pigment.
5. After the film coating weight target is achieved within an acceptable range of variability, the spray system is stopped, and the drying system is continued with periodic slow movement of the coating pan for an additional 10-20 minutes to remove excess water from the tablets. When the coated tablets reach an acceptable residual moisture content determined by at-line testing with a loss on drying moisture balance, the coating process is stopped and the coated tablets are discharged from the coating machine.

TABLE 13.3
composition of tablets

| | | | | Quantity/tablet, |
|---|---|---|---|---|
| Component | Function | Quantity % | Quantity, g | mg |
| Agent 1 granules | Active | 33.3 | 1,000 | 100 |
| Agent 2 granules | Active | 66.7 | 2,000 | 200 |
| Film coating | Cosmetic | 3 | 90 | 9 |
| For example Opadry II | coating | | | |
| White film coating | | | | |
| Totals | | 103% | 3,090 g | 309 mg |

### Example 14: Measuring Serotonin and Dopamine Releasing Effects of Molecules

The serotonin and dopamine releasing effects of molecules may be measured in a number of ways known to those skilled in the arts. These include measuring serotonin increases in plasma, decreased serotonin uptake in cells, serotonin and dopamine release from rat synaptosomes, and serotonin and dopamine release from cells that have human monoamine transporters on their surface. These assay methods are briefly detailed below.

### Serotonin Release Assay Methods Using Plasma

Serum serotonin can be measured using High Performance Liquid Chromatography and Fluorescence Detection. Venipuncture collects at least 1 mL of sample, which is spun with serum frozen to below −20° C. within 2 hours of collection. For active compounds, assay results will show increases in serum serotonin, indicating that the compound is a releaser of serotonin.

### Serotonin Release Assay Using Human Serotonin Transporter (SERT, SLC6A4) in HEK-293 Cells

Human recombinant serotonin transporter expressed in HEK-293 cells are plated. Test compound and/or vehicle is preincubated with cells (1×10E5/ml) in modified Tris-HEPES buffer pH 7.1 for 20 minutes at 25° C. and 65 nM. [³H]Serotonin is then added for an additional 15 minute incubation period. Bound cells are filtered and counted to determine [3H]Serotonin uptake. Compounds are screened at concentrations from 10 to 0.001 μM or similar. Reduction of [³H]Serotonin uptake relative to 1 μM fluoxetine indicates inhibitory activity.

### Serotonin Release and Dopamine Release Assay Methods Using Rat Synaptosomes

An alternative, invasive method of measuring compound interactions with the serotonin, dopamine, or norepinephrine transporter can be conducted according to the methods of Solis et al (2017. Neuropsychopharmacology, 42(10), 1950-1961) and Rothman and Baumann (Partilla et al. 2016. In: Bönisch S, Sitte HH (eds) Neurotransmitter Transporters Springer; New York, pp 41-52).

Male Sprague-Dawley rats (Charles River, Kingston, NY, USA) are used for the synaptosome assays. Rats are group-housed with free access to food and water, under a 12 h light/dark cycle with lights on at 0700 h. Rats are euthanized by CO₂ narcosis, and synaptosomes prepared from brains using standard procedures (Rothman, R. B., & Baumann, M. H. (2003). Monoamine transporters and psychostimulant drugs. European journal of pharmacology, 479(1-3), 23-40). Transporter uptake and release assays are performed as described previously (Solis et al. 2017. Neuropsychopharmacology, 42(10), 1950-1961). In brief, synaptosomes are prepared from caudate tissue for dopamine transporter (DAT) assays, and from whole brain minus caudate and cerebellum for norepinephrine transporter (NET) and serotonin (5-HT) transporter (SERT) assays.

For uptake inhibition assays, 5 nM [³H]dopamine, [³H]norepinephrine, or [³H]5-HT are used for DAT, NET, or SERT assays respectively. To optimize uptake for a single transporter, unlabeled blockers are included to prevent the uptake of [³H]neurotransmitter by competing transporters. Uptake inhibition is initiated by incubating synaptosomes with various doses of test compound and [³H]neurotransmitter in Krebs-phosphate buffer. Uptake assays were terminated by rapid vacuum filtration and retained radioactivity is quantified with liquid scintillation counting (Baumann et al. 2013. Neuropsychopharmacology, 38(4), 552-562).

For release assays, 9 nM [³H]MPP+(1-methyl-4-phenylpyridinium) is used as the radiolabeled substrate for DAT and NET, whereas 5 nM [³H]5-HT is used for SERT. Alternatively [³H]dopamine and [³H]norepinephrine may be used for DAT and NET assays, respectively. All buffers used in the release assay contain 1 μM reserpine to block vesicular uptake of substrates. The selectivity of release assays is optimized for a single transporter by including unlabeled blockers to prevent the uptake of [³H]MPP+ or [³H]5-HT by competing transporters. Synaptosomes are preloaded with radiolabeled substrate in Krebs-phosphate buffer for 1 hr to reach steady state. Release assays are initiated by incubating preloaded synaptosomes with various concentrations of the test drug. Release is terminated by vacuum filtration and retained radioactivity quantified by liquid scintillation counting.

Effects of test drugs on release are expressed as a percent of maximal release, with maximal release (i.e., 100% Emax) defined as the release produced by tyramine or a similar releaser (such as dextroamphetamine at DAT and NET and norfenfluramine at SERT) at doses that evoke the efflux of all ‘releasable’ tritium by synaptosomes (10 μM tyramine for DAT and NET assay conditions, and 100 μM tyramine for SERT assay conditions). Effects of test drugs on release are analyzed by nonlinear regression. Dose-response values for the uptake release are fit to the equation, Y(x)=Ymin+(Ymax−Ymin)/(1+10exp[(logEC₅₀−logx)]×n), where x is the concentration of the compound tested, Y(x) is the response measured, Ymax is the maximal response, EC₅₀ is the concentration that yields half-maximal release, and n is the Hill slope parameter. EC₅₀ₛ for release of less than 10 uM, but often less than 1 uM, are usually considered indicative of substrate-type releasers.

### Serotonin Release Assay Methods Using Human SERT

Chinese hamster ovary cells or similar expressing human SERT are seeded in Cytostar™ (PerkinElmer) plate with standard culture medium the day before the experiment at a single density (5000 cells/assay). Cells are incubated overnight with 5% CO₂ at 37° C. The day of experiment, the medium was replaced by incubation buffer (140 mM NaCl, 4.8 mM KCl, 1.2 mM MgSO₄, 0.1 mM KH2PO₄, 10 mM HEPES, pH 7.4) with a single concentration of [³H]serotonin at 150 nM.

Cells are incubated at room temperature at different incubation times and radioactivity is counted. Test compounds are measured at concentrations of 1e-10, 1e-09, 1e-08,1e-07,1e-06, 1e-05, and 1e-04 M or similar.

EC/IC_{50S} can be calculated using the R packages drm (to fit the regression model) and LL.4 (to define the structure of the log-logistic regression model) or similar statistical software. Values are fit to the following function: 10 f(x)=c+(d−c)/(1+exp(b (log(x)−log(e))) where b=the Hill coefficient, c=minimum value, d=maximum value, and e=EC₅₀/IC₅₀.

### Dopamine-Release Assay Methods Using Human DAT

Chinese hamster ovary cells or similar expressing human DAT are seeded in Cytostar™ plate with standard culture medium the day before experiment at one single density (2500 cells/assay). Cells are incubated overnight with 5% CO₂ at 37° C. The day of experiment, the medium is replaced by incubation buffer (TrisHCl 5 mM, 120 mM NaCl, 5.4 mM KCl, 1.2 mM MgSO₄, 1.2 mM CaCl₂), Glucose 5 mM, 7.5 mM HEPES, pH 7.4) with a single concentration of [³H]dopamine at 300 nM. In control wells, the specificity of DAT uptake is verified by adding the reference control GBR 12909 (10 μM). For all assays, three reference conditions are employed: (1) radioligand-containing buffer only, to verify the control level of release, (2) buffer with 1% DMSO (solvent used to solubilize the test compounds), (3) 100 μM amphetamine (in 1% DMSO) to make it possible to calculate a relative Eₘₐₓ.

Cells are incubated at room temperature at different incubation times and radioactivity is counted. Test compounds are measured at concentrations of 1e-10, 1e-09, 1e-08,1e-07,1e-06, 1e-05, and 1e-04 M or similar.

EC/IC_{50S} can be calculated using the R packages drm (to fit the regression model) and LL.4 (to define the structure of the log-logistic regression model) or similar statistical software. Values are fit to the following function: 10 f(x)=c+(d−c)/(1+exp(b (log(x)−log(e))) where b=the Hill coefficient, c=minimum value, d=maximum value, and e=EC₅₀/IC₅₀.

### Example 15: Evaluation of Therapeutic Profile of Entactogens in Humans

The methods and compositions disclosed herein can be evaluated by measuring desirable and undesirable effects of entactogens and showing that the ratio of the two is increased. Desirable effects of an entactogen include decreased neuroticism, increased authenticity, and positive mood (including often feelings of elation, emotional closeness to others, and love). Additionally, decreased changes in blood pressure and heart rate are also desirable.

If the entactogen is administered to a person with some mental health condition, then standard measures for assessing the severity of that condition may also be used to show therapeutic benefits. For example, the Clinician-Administered PTSD Scale for DSM-5 (CAPS-5) may be used to measure therapeutic benefits in an individual with PTSD.

The entactogenic effect of decreased neuroticism can be measured as a decrease in social anxiety using the Brief Fear of Negative Evaluation-revised (BFNE) (Carleton et al., 2006, Depression and Anxiety, 23(5), 297-303; Leary, 1983, Personality and Social Psychology bulletin, 9(3), 371-375). This 12-item Likert scale questionnaire measures apprehension and distress due to concerns about being judged disparagingly or with hostility by others. Ratings use a five-point Likert scale with the lowest, middle, and highest values labeled with “much less than normal,” “normal,” and “much more than normal.” The BFNE can be administered before and repeatedly during therapeutic drug effects. Participants are instructed to answer how they have been feeling for the past hour, or otherwise during the effect of the drug. Baseline-subtracted responses are typically used in statistical models.

The entactogenic effect of authenticity can be measured using the Authenticity Inventory (Kernis & Goldman. 2006. Advances in experimental social psychology, 38, 283-357) as modified by Baggott et al (Journal of Psychopharmacology 2016, 30.4: 378-87). Administration and scoring of the instrument are described by Baggott et al (ibid) and are almost identical to that of the BFNE.

Entactogenic effects on mood can be measured using visual analog scales and other questionnaires common in the clinical research literature (see methods used in Baggott et al., ibid; Holze et al. 2020. Neuropsychopharmacology, 45(3), pp. 462-471; Regan et al. 2021. PloS one, 16(10), p.e025884; Studerus et al. 2021. Journal of Psychopharmacology, p.0269881121998322 and references therein). Items of particular interest relate to positively valenced experiences and feelings of elation, emotional openness, emotional closeness to others, and love.

Acute physiological changes can be measured in humans with standard clinical methods (blood pressure cuffs, 3-lead EKG, tympanic or oral temperature, serum sodium, etc), with measures usually collected before and at scheduled intervals after an entactogen. For example, measures may be collected before, 0.5, 0.75, 1, 1.5, 2, 2.5, 3, 4, 5, 6, and 8 hours after an entactogen. Maximum change from baseline and area-under-the-effects-versus-time-curve may be used as summary measures and statistically compared to a placebo control condition.

Undesirable symptoms of an entactogen include nausea, vomiting, headache, sedation, difficulty concentrating, lack of appetite, lack of energy, and decreased mood. Additional symptoms are detailed by Vizeli and Liechti (2017. Journal of Psychopharmacology, 31(5), pp. 576-588) and in the MDMA Investigator's Brochure, 14th Edition: Mar. 18, 2022, and references therein, available from the sponsor of MDMA clinical trials at MAPS.org. To measure these and other undesirable effects, patients can be asked to complete a self-report symptom questionnaire, such as the Subjective Drug Effects Questionnaire (SDEQ) or List of Complaints. The SDEQ is a 272-item self-report instrument measuring perceptual, mood, and somatic changes caused by drugs including hallucinogens like LSD (Katz et al. 1968. J Abnorm Psychology 73:1-14). It has also been used to measure the therapeutic and adverse effects of MDMA (Harris et al. 2002. Psychopharmacology, 162(4), 396-405). The List of Complaints is a 66-item questionnaire that measures physical and general discomfort and is sensitive to entactogen-related complaints (e.g., Vizeli & Liechti. 2017. Journal of Psychopharmacology, 31(5), 576-588).

Alternatively, individual items can be taken from the SDEQ or List of Complaints in order to create more focused questionnaires and reduce the burden of filling out time-consuming paperwork on participants.

In some embodiments, a symptom questionnaire is administered approximately 7 hours after a patient takes an entactogen (with instructions to answer for the time since taking the entactogen) and then daily (with instructions to answer for the last 24 hours) for up to 96 hours after the entactogen was taken. Decreases in adverse effects of a formulation compared to standard MDMA can be shown by comparing the intensity or prevalence of effects that occur. Prevalence of adverse effects including headache, difficulty concentrating, lack of appetite, lack of energy, and decreased mood may be decreased by approximately 10%, 20%, 30%, 40%, 50%, 60%, 70%, 80%, 90%, 99%, or 100%.

### Example 16: Evaluation of Therapeutic Profile of Entactogens in Rodents

As an alternative to measuring the therapeutic profile of entactogens in clinical trials, preclinical studies in rodents may also be used. Here, the logic is similar as in the clinical case with desired effects and undesired effects measured. The primary difference is that drug administration typically uses parenteral administration with doses and timing designed to imitate the initial pharmacokinetics of the human case. Thus, rather than, for example, controlled release after oral administration, it makes sense to discuss and evaluate exposure regimens that produce equivalent Tₘₐₓ differences.

Appropriate tasks and behaviors that may be used to measure undesired effects include physiological measures (heart rate, blood pressure, body temperature), the modified Irwin procedure or functional observational battery (Irwin, Psychopharmacologia, 13, 222-257, 1968), and locomotor activity (such as distance traveled, rearing frequency, and rearing duration; Piper et la., J Pharmacol Exp Ther, 317, 838-849, 2006). In these studies, an entactogen is administered at different doses (including a vehicle only placebo) to different groups of animals and measures are made at scheduled times before and after administration. For example, 0, 1.5, 3, 15, and 30 mg/kg of a compound may be administered, and measures made before and 15, 30, 60, 120 and 180 minutes and 12, 24, 36, and 48 hours after administration of the test substance.

The modified Irwin procedure or the functional observational battery (FOB) can be used compare different exposure profiles to standard exposure profiles (Mathiasen and Moser, 2018. Current protocols in pharmacology, 83(1), p.e43; Redfern et al. 2019. Journal of pharmacological and toxicological methods, 98, p.106591; Roux, Sable, and Porsolt, 2004. Current protocols in pharmacology, 27(1), pp. 1-23), with an estimated therapeutic index being suggested by the doses that induces marked undesirable nervous system behaviors in comparison to therapeutic doses. Dose ranges are selected to determine a no effect level and lowest observed adverse effect levels.

To estimate effective therapeutic dose, a drug discrimination procedure can be used. Rodent drug discrimination assays are the main methodology for understanding the interoceptive (i.e., felt experience) effects of drugs in animals (Baker, 2017. Neurobiology of Psychedelic Drugs, 201-219; Fantegrossi, Murnane & Reissig. 2008. Biochemical pharmacology, 75(1), 17-33). In a typical discrimination task, an animal is trained to emit one response (often pressing of a lever) during experimental sessions shortly after the administration of a particular drug (the “training drug”), and a different response during sessions that follow administration of the placebo drug vehicle. Once the animals are stably engaged in this task, a novel drug can be tested for similarity to the training drug.

Training and testing procedures are conducted in standard operant conditioning chambers housed in sound-attenuating shells. Dustless Precision Pellets (45 mg; Product #F0021; BioServ, Flemington, NJ) are used as reinforcements for lever pressing.

A training dose of 0.5 to 2.0 mg/kg MDMA is used, and training follows standard procedures (Baker 2017, Neurobiology of Psychedelic Drugs, 201-219). Briefly, rats are trained to discriminate 1.5 mg/kg MDMA from placebo (saline vehicle) under a fixed ratio (FR) 20 schedule of food reinforcement. Lever assignment to stimulus condition is counterbalanced among rats in each experiment. Drug and vehicle training sessions are alternated with the order guaranteeing that the same stimulus condition occurs no more than twice consecutively. The criterion for stimulus control is a minimum of eight out of ten consecutive discrimination training sessions with 80% or higher correct lever responses prior to delivery of the first reinforcer and for the total session.

After stimulus control is established, test sessions are conducted. The test compound is administered to produce the desired release profile prior to starting the test sessions. Test sessions are similar to training sessions, with the exception that responses are not reinforced and sessions end upon completion of the first FR 20 or after 20 min, whichever occurs first. Testing criteria between sessions require subjects to complete at least one drug and at least one vehicle training session consecutively with 80% or higher injection-appropriate responding. The main outcome measure is percent responses on the MDMA-associated lever, while rate of lever pressing (responses per second) is used as a control measure. After a range of doses are administered, it is possible to estimate an EC50 for discrimination. In one embodiment, the EC50 for discrimination is lowered while the lowest observed adverse effect level is unchanged or raised. In another embodiment, the EC50 for discrimination is unchanged while the lowest observed adverse effect level is raised.

While the present invention is described in terms of particular embodiments and applications, it is not intended that these descriptions in any way limit its scope to any such embodiments and applications, and it will be understood that many modifications, substitutions, changes, and variations in the described embodiments, applications, and details of the invention illustrated herein can be made by those skilled in the art without departing from the spirit of the invention, or the scope of the invention as described in the appended claims.

We claim:

1. A pharmaceutical composition comprising
(a) immediate release granules comprising a dopamine releasing agent and one or more pharmaceutically acceptable excipients;
(b) delayed release granules comprising an entactogen selected from the group consisting of: 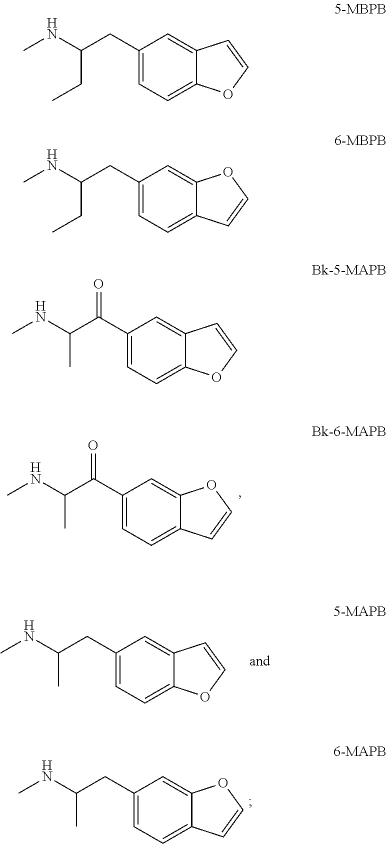
and one or more pharmaceutically acceptable excipients; and
(c) one or more additional pharmaceutical excipients;
wherein:
the dopamine releasing agent has a dopamine release EC₅₀ of less than 10 μM;
the pharmaceutical composition provides a kinetic lag between about 15 and 240 minutes; and
the kinetic lag is characterized by the dopamine releasing agent having a more rapid onset of therapeutic effects than the entactogen.

2. The pharmaceutical composition of claim 1, wherein the dopamine releasing agent is amphetamine, fencamfamine, phenmetrazine, 2-fluorophenmetrazine, 3-fluorophenmetrazine, metamnetamine (methyl[1-(5,6,7,8-tetrahydronaphthalen-2-yl)propan-2-yl]amine), naphthylaminopropane, 5-(2-Aminopropyl)indole, or methcathinone.

3. The pharmaceutical composition of claim 1, wherein the dopamine releasing agent is 2-methyl-methcathinone, 3-methyl-methcathinone, 4-methyl-methcathinone (4-MMC), 3-fluoroamphetamine, 3-fluoromethcathinone, 4-fluoroamphetamine, 4-fluoromethcathinone, or 3-bromoamphetamine.

4. The pharmaceutical composition of claim 1, wherein the dopamine releasing agent is 3-bromomethcathinone, 4-bromoamphetamine, 4-bromomethcathinone, N-methylamphetamine, N-benzyl-methamphetamine, 3-methylamphetamine, or 4-methylamphetamine.

5. The pharmaceutical composition of claim 1, wherein the dopamine releasing agent is N,4-dimethylamphetamine, 2-(Methylamino)-1-naphthalen-1-ylpropan-1-one, methylthioamphetamine, or N,N-dimethyl-thioamphetamine.

6. The pharmaceutical composition of claim 1, wherein the dopamine releasing agent has a dopamine release EC₅₀ of less than 1 μM.

7. The pharmaceutical composition of claim 1, wherein the dopamine releasing agent has a dopamine release EC₅₀ of less than 250 nM.

8. The pharmaceutical composition of claim 1, wherein the dopamine releasing agent has a DAT to SERT EC₅₀ ratio that is at least two times greater than the than the DAT to SERT EC₅₀ ratio for the entactogenic compound.

9. The pharmaceutical composition of claim 1, wherein the dopamine releasing agent has a lower DAT EC₅₀ than the entactogenic compound.

10. The pharmaceutical composition of claim 1, wherein the entactogenic compound has at least two times greater DAT EC₅₀ than the dopamine releasing agent.

11. The pharmaceutical composition of claim 1, wherein the kinetic lag is a difference in Tmax of the two agents.

12. The pharmaceutical composition of claim 11, wherein the difference in Tmax of the agents is less than about 90 minutes.

13. The pharmaceutical composition of claim 1, wherein the kinetic lag is a difference in timing of the 50% Cmax of the two agents.

14. The pharmaceutical composition of claim 13, wherein the difference in timing of the 50% Cmax of the agents is less than about 90 minutes.

15. The pharmaceutical composition of claim 1, wherein the kinetic lag is a difference in Tpeak of the two agents.

16. The pharmaceutical composition of claim 15, wherein the difference in Tpeak of the agents is less than about 120 minutes.

17. The pharmaceutical composition of claim 1, wherein the entactogen is 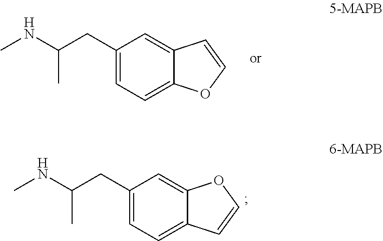
or a pharmaceutically acceptable salt or salt mixture thereof.

18. The pharmaceutical composition of claim 1, wherein the entactogen is 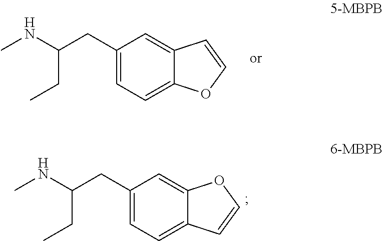
or a pharmaceutically acceptable salt or salt mixture thereof.

19. The pharmaceutical composition of claim 1, wherein the entactogen is 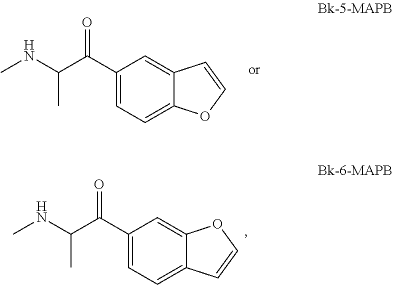
or a pharmaceutically acceptable salt or salt mixture thereof.
